(19) United States
(12) Patent Application Publication
(10) Pub. No.: US 2026/0143900 A1
(43) Pub. Date: May 21, 2026

(54) HETEROCYCLIC COMPOUND, COMPOSITION FOR ORGANIC LAYER INCLUDING THE SAME, AND LIGHT EMITTING DEVICE INCLUDING THE SAME

(71) Applicant: LT MATERIALS CO., LTD., Gyeonggi-do (KR)

(72) Inventors: SOL LEE, Gyeonggi-do (KR); JUN TAE MO, Gyeonggi-do (KR); DONG JUN KIM, Gyeonggi-do (KR); DAE HYUK CHOI, Gyeonggi-do (KR)

(21) Appl. No.: 19/393,916

(22) Filed: Nov. 19, 2025

(30) Foreign Application Priority Data
Nov. 19, 2024 (KR) 10-2024-0164789

(51) Int. Cl. H10K 50/12; C07B 59/00; C07D 307/91; H10K 50/15; H10K 50/16; H10K 50/19; H10K 85/60

(52) U.S. Cl. CPC H10K 50/12; C07B 59/002; C07D 307/91; H10K 50/15; H10K 50/16; H10K 50/19; H10K 85/626; H10K 85/633; H10K 85/636; H10K 85/654; H10K 85/6572; H10K 85/6574; H10K 85/6576; C07B 2200/05

(57) ABSTRACT

A heterocyclic compound is represented by Formula 1. A light-emitting device includes a first electrode, a second electrode disposed on the first electrode, and one or more organic layers interposed between the first electrode and the second electrode. One or more of the organic layers includes the heterocyclic compound. One or more of the organic layer may further include a compound represented by Formula 3.

### CROSS-REFERENCE TO RELATED APPLICATION AND CLAIM OF PRIORITY

This application claims the benefit under 35 USC § 119 of Korean Patent Application No. 10-2024-0164789, filed on Nov. 19, 2024, in the Korean Intellectual Property Office, the entire disclosure of which is incorporated herein by reference for all purposes.

### BACKGROUND

### 1. Field of the Invention

The present disclosure relates to a heterocyclic compound, a composition for an organic layer including the compound, and a light-emitting device including the composition.

### 2. Description of the Related Art

Organic light-emitting displays (OLEDs) include organic light-emitting diodes (OLEDs) that exhibit self-luminescence. Because they do not require a separate light source, OLED displays offer wide viewing angles and fast response times, and can enhance contrast and brightness.

In OLEDs, an organic light-emitting layer is formed for each pixel, and the organic light-emitting layer can be sandwiched between opposing electrodes. Holes and electrons injected from each electrode recombine in the organic light-emitting layer to generate excitons, and the energy released by these excitons can generate light.

Research is being conducted on materials applicable to the organic light-emitting layer to achieve high-efficiency and long-life OLEDs.

### SUMMARY

A heterocyclic compound represented by Formula 1 according to the present disclosure below.

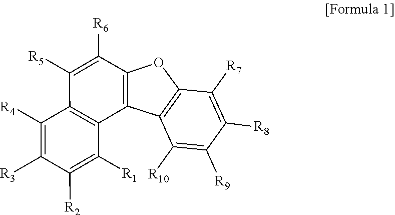

In Formula 1, one of R₁ to R₄ is an N-Het represented by Formula 2 below, one of R₅ to R₁₀ is Ar₁,

Ar₁ is a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

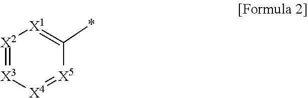

in Formula 2, X₁ to X₅ are each independently N or CR₁₁,

R₁₁ is selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms, provided that at least two of X₁ to X₅ are N,
the remainders of R₁ to R₄ that are not N-Het and the remainders of R₅ to R₁₀ that are not Ar₁ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRRR″; and —P(═O)RR′, or adjacent groups are linked to form a substituted or unsubstituted 5- to 9-membered saturated or unsaturated ring,

R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and
* represents a bonding site to an adjacent atom.

According to exemplary embodiments, a light-emitting device may include a heterocyclic compound of one embodiment. The heterocyclic compound described above may, for example, function as a host material to control the energy band gap and energy levels of the light-emitting layer. Therefore, the light-emitting efficiency and lifespan of the light-emitting device may be improved by the heterocyclic compound described above.

### BRIEF DESCRIPTION OF THE DRAWINGS

The above and other objects, features and advantages of the present disclosure will be more clearly understood from the following detailed description taken in conjunction with the accompanying drawings, in which:

FIGS. 1 to 4 are views schematically illustrating laminated structures of a light-emitting device according to one embodiment of the present disclosure, respectively.

### DETAILED DESCRIPTION OF THE INVENTION

Hereinafter, exemplary embodiments of the present disclosure will be described in detail with reference to the accompanying drawings. However, these embodiments are merely illustrative, and the present disclosure is not limited to the specific embodiments described as examples.

As used herein, when a portion is described to “include” a component, unless otherwise specified, it means that the portion does not exclude other components and may further include other components.

As used herein, the * symbol in a chemical formula represents a bonding site.

As used herein, “substitution” means that any hydrogen atom bonded to a carbon atom in a compound is replaced with another substituent. The position of substitution is not limited as long as the substituent is at a substitutable position. If two or more substitutions are made, the two or more substituents may be the same or different.

As used herein, “substituted or unsubstituted” refers to a state in which at least one hydrogen atom of a compound is unsubstituted or substituted with one or more substituents selected from the group consisting of deuterium; a halogen group; —CN; an alkyl group having 1 to 60 carbon atoms; an alkenyl group having 2 to 60 carbon atoms; an alkynyl group having 2 to 60 carbon atoms; a haloalkyl group having 1 to 60 carbon atoms; an alkoxy group having 1 to 60 carbon atoms; an aryloxy group having 6 to 60 carbon atoms; an alkylthio group having 1 to 60 carbon atoms; an arylthio group having 6 to 60 carbon atoms; an alkylsulfinyl group having 1 to 60 carbon atoms; an arylsulfinyl group having 6 to 60 carbon atoms; a cycloalkyl group having 3 to 60 carbon atoms; a heterocycloalkyl group having 2 to 60 carbon atoms; an aryl group having 6 to 60 carbon atoms; a heteroaryl group having 2 to 60 carbon atoms; —SiRR′R″; —P(═O)RR′; and —NRR′, or substituted with a substituent in which two or more of the foregoing substituents are linked. R, R′ and R″ are each independently a substituent including at least one of hydrogen; deuterium; a halogen group; an alkyl group; an alkenyl group; an alkoxy group; a cycloalkyl group; a heterocycloalkyl group; an aryl group; and a heteroaryl group.

As used herein, when a substituent is not indicated in a chemical formula or compound structure, it means that a hydrogen atom is bonded to the carbon atom. However, since deuterium (2H) is an isotope of hydrogen, some hydrogen atoms may be deuterium.

As used herein, when a substituent is not indicated in a chemical formula or compound structure, it may mean that all positions that can be substituted are hydrogen or deuterium. In other words, deuterium is an isotope of hydrogen, and some of the hydrogen atoms may be deuterium atoms, in which case the deuterium content may be 0% to 100%.

When a substituent is not indicated in a chemical formula or compound structure, and the deuterium content is 0%, the hydrogen content is 100%, and all substituents are hydrogen, hydrogen and deuterium may nevertheless be present together in the compound unless deuterium is explicitly excluded.

Deuterium is an isotope of hydrogen, having a nucleus composed of one proton and one neutron (a deuteron). It may be represented as hydrogen-2 and its elemental symbol may be written as D or ²H.

Isotopes are atoms with the same atomic number (Z) but different mass numbers (A). Isotopes may also be defined as atoms with the same number of protons but different numbers of neutrons.

As used herein, the content T % of a specific substituent is defined as T2/T1×100=T(%), where the total number of substituents that a basic compound may have is defined as T1, and the number of the specific substituent among them is defined as T2.

In one example, a 20% deuterium content in a phenyl group represented by

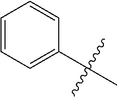 [SMILES: *c1ccccc1]

means that the total number of substituents that the phenyl group may have is 5 (T1 in the formula), and among them, the number of deuterium atoms is 1 (T2 in the formula), which may be represented as 20%. That is, cases where the deuterium content in the phenyl group is 20% may be represented by the structural formula below.

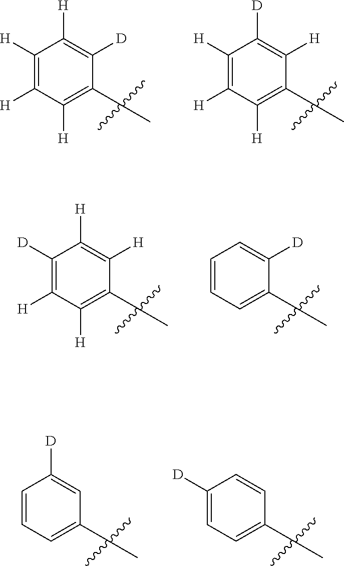 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, “a phenyl group having 0% deuterium content” may refer to a phenyl group that includes no deuterium atoms and has five hydrogen atoms.

As used herein, halogen may be fluorine, chlorine, bromine, or iodine.

As used herein, an alkyl group includes a straight or branched chain having 1 to 60 carbon atoms, and may be further substituted with one or more substituents. The alkyl group may have 1 to 60, 1 to 40, or 1 to 20 carbon atoms. Examples thereof may include a methyl group, an ethyl group, a propyl group, an n-propyl group, an isopropyl group, a butyl group, an n-butyl group, an isobutyl group, a tert-butyl group, a sec-butyl group, a 1-methylbutyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethylpropyl group, a 1,1-dimethylpropyl group, an isohexyl group, a 2-methylpentyl group, a 4-methylhexyl group, and a 5-methylhexyl group, but are not limited thereto.

As used herein, the alkenyl group includes a straight or branched chain having 2 to 60 carbon atoms, and may be further substituted with one or more substituents. The alkenyl group may have 2 to 60, 2 to 40, or 2 to 20 carbon atoms. Examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl group, a 2-phenylvinyl group, a 2,2-diphenylvinyl group, a 2-phenyl-2-(naphthyl-1-yl) vinyl group, a 2,2-bis(diphenyl) vinyl group, a stilbenyl group, and a styrenyl group, but are not limited thereto.

As used herein, an alkynyl group includes a straight or branched chain having 2 to 60 carbon atoms, and may be further substituted with one or more substituents. The alkynyl group may have 2 to 60, 2 to 40, or 2 to 20 carbon atoms.

As used herein, a haloalkyl group refers to an alkyl group substituted with a halogen group. Examples thereof may include —CF₃ and —CF₂CF₃, but are not limited thereto.

As used herein, a cycloalkyl group includes a monocyclic or polycyclic group having 3 to 60 carbon atoms, and may be further substituted with one or more substituents. In this context, a polycyclic group refers to a group in which a cycloalkyl group is directly linked to or condensed with another cyclic group. The other cyclic group may be a cycloalkyl group, but may also be another type of cyclic group, such as a heterocycloalkyl group, an aryl group, or a heteroaryl group. The number of carbon atoms in the cycloalkyl group may be 3 to 60, 3 to 40, or 5 to 20. Examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, and a cyclooctyl group, but are not limited thereto.

As used herein, a heterocycloalkyl group includes a monocyclic or polycyclic group having 2 to 60 carbon atoms and including at least one heteroatom selected from O, S, Se, N and Si, and may be further substituted with one or more substituents. In this context, the term “polycyclic” refers to a group in which a heterocycloalkyl group is directly linked to or condensed with another ring group. The other ring group may be a heterocycloalkyl group, but may also be another type of ring group, such as a cycloalkyl group, an aryl group, or a heteroaryl group. The heterocycloalkyl group may have 2 to 60, 2 to 40, or 3 to 20 carbon atoms.

As used herein, an aryl group includes a monocyclic or polycyclic group having 6 to 60 carbon atoms, and may be further substituted with one or more substituents. In this context, the term “polycyclic” refers to a group in which an aryl group is directly linked to or condensed with another ring group. The other ring group may be an aryl group, but may also be another type of ring group, such as a cycloalkyl group, a heterocycloalkyl group, or a heteroaryl group. The aryl group may include a spiro ring structure. The aryl group may have 6 to 60, 6 to 40, or 6 to 25 carbon atoms. Specific examples of the aryl group may include a phenyl group, a biphenyl group, a terphenyl group, a naphthyl group, an anthracenyl group, a chrysenyl group, a phenanthryl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, and condensed ring groups thereof, but are not limited thereto.

As used herein, the terphenyl group may be selected from the following structures.

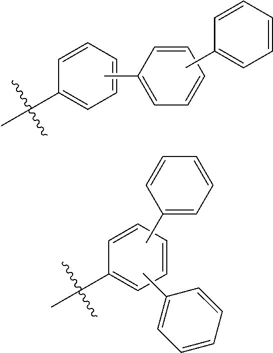

As used herein, the fluorenyl group may be substituted, and adjacent substituents may be bonded to form a ring.

The substituted fluorenyl group may be represented by the following structural formula, but is not limited thereto.

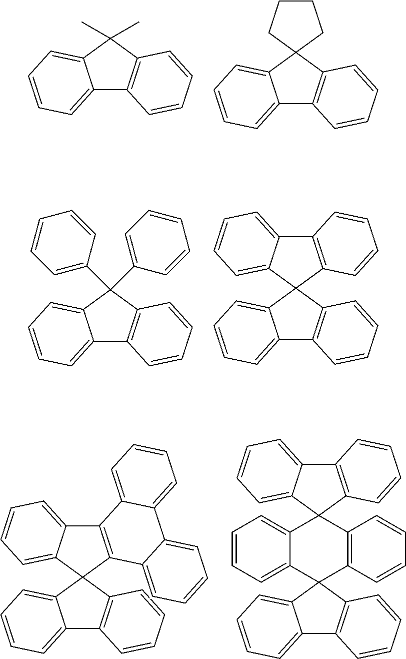

As used herein, an alkoxy group is represented by —O(R101), and R101 may be selected from the examples of the alkyl group described above.

As used herein, an aryloxy group is represented by —O(R102), and R102 may be selected from the examples of the aryl group described above.

As used herein, an alkylthio group is represented by —S(R103), and R103 may be selected from the examples of the alkyl group described above.

As used herein, an arylthio group is represented by —S(R104), and R104 may be selected from the examples of the aryl group described above.

As used herein, an alkylsulfinyl group is represented by —S(O)₂(R105), and R105 may be selected from the examples of the alkyl group described above.

As used herein, an arylsulfinyl group is represented by —S(═O)/(R106), and R106 may be selected from the examples of the aryl group described above.

As used herein, a heteroaryl group includes a monocyclic or polycyclic group having 2 to 60 carbon atoms and including at least one heteroatom selected from S, O, Se, N, and Si, and may be further substituted with one or more substituents. In this context, the term “polycyclic” refers to a group in which a heteroaryl group is directly linked to or condensed with another ring group. The other ring group may be a heteroaryl group, but may also be another type of ring group, such as a cycloalkyl group, a heterocycloalkyl group, or an aryl group. The heteroaryl group may have 2 to 60, 2 to 40, or 3 to 25 carbon atoms. Examples of the heteroaryl group may include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazolyl group, a benzocarbazolyl group, a dibenzocarbazolyl group, a phenazine group, a dibenzosilole group, a spirobi (dibenzosilole) group, a dihydrophenazine group, a phenoxazine group, a phenanthridine group, a thiophenyl group, an indolo[2,3-a]carbazolyl group, an indolo[2,3-b]carbazolyl group, an indoline group, a 10,11-dihydrodibenzo[b,f]azepine group, a 9,10-dihydroacridine group, a phenanthrazine group, a phenothiathiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azacillin group, a pyrazolo[1,5-c]quinazoline group, a pyrido[1,2-b]indazole group, a pyrido[1,2-a]imidazo[1,2-e]indolin group, and a 5,11-dihydroindeno[1,2-b]carbazolyl group, but are not limited thereto.

As used herein, when a substituent is a carbazolyl group, it means that the substituent is bonded to the nitrogen or carbon of the carbazolyl group.

As used herein, when a carbazolyl group is substituted, an additional substituent may be present on the nitrogen or carbon of the carbazolyl group.

As used herein, examples of a benzocarbazolyl group may include any one of the following structures.

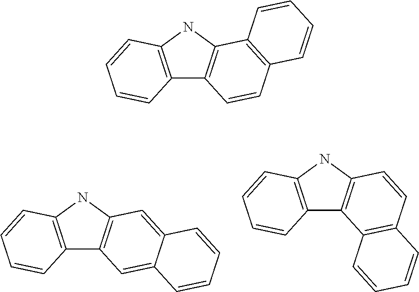

As used herein, examples of a dibenzocarbazolyl group may include any one of the following structures.

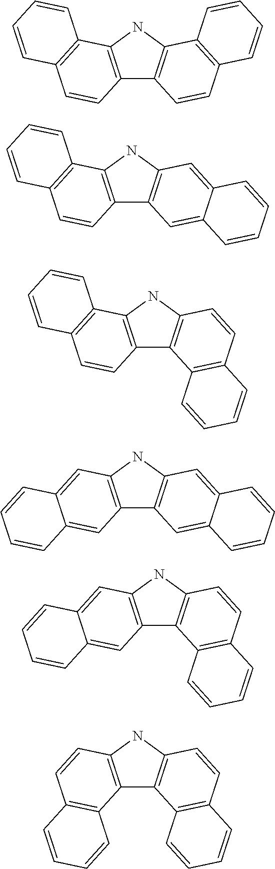

As used herein, examples of a naphthobenzofuran group may include any one of the following structures.

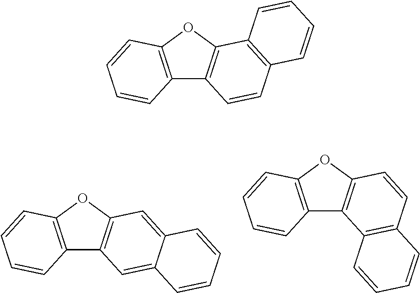

As used herein, examples of a naphthobenzothiophene group may include any one of the following structures.

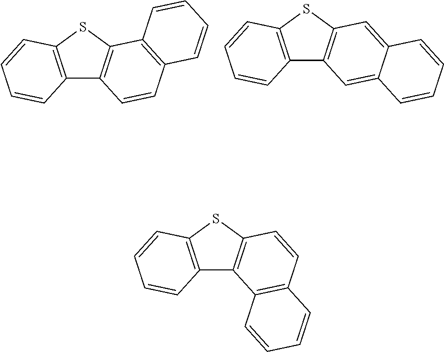

Among the substituents, —SiRR′R″ is a silyl group, which is a substituent that includes Si, and the Si atom is directly bonded as a radical. R, R′ and R″ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. Examples of the silyl group may include,

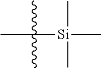 [SMILES: *[Si](C)(C)C]

(trimethylsilyl group),

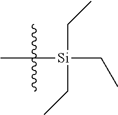 [SMILES: *[Si](CC)(CC)CC]

(triethylsilyl group),

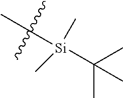 [SMILES: *[Si](C)(C)C(C)(C)C]

(t-butyldimethylsilyl group),

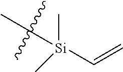 [SMILES: *[Si](C)(C)C=C]

(vinyldimethylsilyl group),

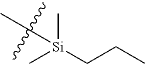 [SMILES: *[Si](C)(C)CCC]

(propyldimethylsilyl group), (triphenylsilyl group)

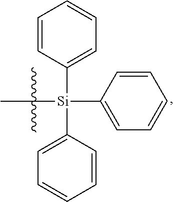 [SMILES: *[Si](c1ccccc1)(c1ccccc1)c1ccccc1]

(diphenylsilyl group),

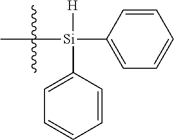 [SMILES: *[SiH](c1ccccc1)c1ccccc1]

and

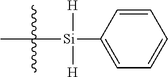 [SMILES: *[SiH2]c1ccccc1]

(phenylsilyl group), but are not limited thereto.

Among the substituents, —P(—O) RR′ is a phosphine oxide group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group, and in particular may be an alkyl group or an aryl group. The alkyl group and aryl group may be selected from the examples described above. For example, the phosphine oxide group may include a dimethylphosphine oxide group, a diphenylphosphine oxide group, and a dinaphthylphosphine oxide group, but is not limited thereto.

Among the substituents, —NRR′ is an amine group, wherein R and R′ may be the same or different, and may each independently be a substituent including at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂, a monoalkylamine group, a monoarylamine group, a monoheteroarylamine group, a dialkylamine group, a diarylamine group, a diheteroarylamine group, an alkylarylamine group, an alkylheteroarylamine group, and an arylheteroarylamine group, and the number of carbon atoms is not particularly limited, but is preferably 1 to 30. Examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methylanthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, and a biphenyltriphenylenylamine group, but are not limited thereto.

As used herein, an arylene group may be selected from the examples of the aryl group described above, except that it is divalent.

As used herein, a heteroarylene group may be selected from the examples of the heteroaryl group described above, except that it is divalent.

As used herein, the term “adjacent” group may refer to a substituent that is substituted on an atom directly linked to the atom substituted by the substituent, a substituent that is sterically closest to the substituent, or another substituent substituted on the atom substituted by the substituent. For example, two substituents substituted at the ortho positions of a benzene ring and two substituents substituted on the same carbon atom of an aliphatic ring may be interpreted as “adjacent” groups.

Hydrocarbon rings and heterocycles that can be formed by adjacent groups may include aliphatic hydrocarbon rings, aromatic hydrocarbon rings, aliphatic heterocycles, and aromatic heterocycles, and the structures exemplified by the above-described cycloalkyl groups, aryl groups, heterocycloalkyl groups, and heteroaryl groups may apply thereto, except that they are not monovalent.

Generally, compounds bonded to hydrogen and compounds substituted with deuterium exhibit different thermodynamic behaviors. The reason is that the mass of the deuterium atom is twice that of hydrogen. Due to this difference in atomic mass, deuterium has the characteristic of having lower vibrational energy.

In addition, the single bond dissociation energy (BDE) of carbon-deuterium is higher than that of carbon-hydrogen. Therefore, structures substituted with deuterium have increased thermal stability, resulting in improved lifetime of devices utilizing them.

When a compound is deposited on a silicon wafer, substances including deuterium tend to exhibit denser intermolecular packing. In addition, observation of the thin film surface with an atomic force microscope (AFM) reveals that thin films formed from deuterium-containing compounds are deposited with a more uniform surface and without aggregation.

The heterocyclic compound of Formula 1 of the present disclosure has a deuterium substitution ratio of greater than 0% and not more than 100%. When deuterium is substituted, the ground state energy is lowered compared to hydrogen-substituted compounds. Furthermore, as the carbon-deuterium bond length decreases, the molecular hardcore volume decreases. This can reduce electrical polarizability and weaken intermolecular interactions, resulting in a more stable stacking structure during device fabrication.

These characteristics create an amorphous state in the thin film, thereby lowering the crystallinity. In other words, the heterocyclic compound of Formula 1 may be effective in improving the heat resistance of OLED devices, thereby enhancing their lifetime and operating characteristics.

The heterocyclic compound according to the present disclosure may be represented by Formula 1 below.

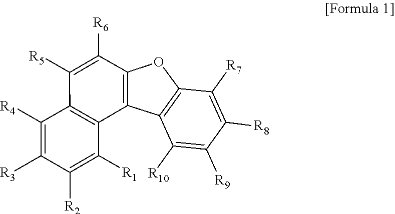

In Formula 1, one of R₁ to R₄ is an N-Het represented by Formula 2 below, one of R₅ to R₁₀ is Ar₁,

Ar₁ is a substituted or unsubstituted aryl group having 6 to 60 carbon atoms; a substituted or unsubstituted oxygen-containing heterocycloalkyl group having 2 to 60 carbon atoms; a substituted or unsubstituted sulfur-containing heterocycloalkyl group having 2 to 60 carbon atoms; a substituted or unsubstituted oxygen-containing heteroaryl group having 2 to 60 carbon atoms; or a substituted or unsubstituted sulfur-containing heteroaryl group having 2 to 60 carbon atoms.

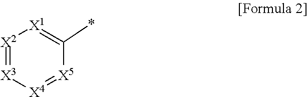

In Formula 2, X₁ to X₅ are each independently N or CR₁₁,

R₁₁ is selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms, and

R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms,

provided that at least two of X₁ to X₅ are N,

the remainders of R₁ to R₄ that are not N-Het and the remainders of R₅ to R₁₀ that are not Ar₁ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and −P(═O)RR′, or adjacent groups are linked to form a substituted or unsubstituted 5- to 9-membered saturated or unsaturated ring,

R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms,

* is a bonding site to an adjacent atom, and

when Ar₁ is a substituted or unsubstituted oxygen-containing heterocycloalkyl group having 2 to 60 carbon atoms; a substituted or unsubstituted sulfur-containing heterocycloalkyl group having 2 to 60 carbon atoms; a substituted or unsubstituted oxygen-containing heteroaryl group having 2 to 60 carbon atoms; or a substituted or unsubstituted sulfur-containing heteroaryl group having 2 to 60 carbon atoms, the N-Het group does not include oxygen or sulfur.

According to exemplary embodiments, in Formula 1, one of R₁ to R₄ is an N-Het represented by Formula 2 below.

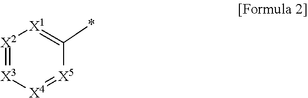

In Formula 2, X₁ to X₅ are each independently N or CR₁₁, provided that at least two of X₁ to X₅ are N. The N-Het may be a triazine-based substituent, a pyrimidine-based substituent, or a quinoxaline-based substituent.

In exemplary embodiments, X₁, X₃, and X₅ may be N. In exemplary embodiments, X₁ and X₃; X₁ and X₅; X₁ and X₄; or X₃ and X₅ may be N.

Any of X₁ to Xₛ that is not N may be CR₁₁. R₁₁ may be hydrogen or a substituent. As used herein, the term “substituent” refers to a group selected from the group consisting of deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′. R, R′ and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In exemplary embodiments, R₁₁ may each independently be hydrogen; deuterium; a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

In exemplary embodiments, adjacent R₁₁ groups may be linked to each other to form a ring. In some embodiments, adjacent R₁₁ groups may be linked to each other to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

For example, adjacent R₁₁ groups may be linked to each other to form a substituted or unsubstituted aryl group having 6 to 40 carbon atoms; a substituted or unsubstituted heterocycloalkyl group having 2 to 40 carbon atoms; or a substituted or unsubstituted heteroaryl group having 2 to 40 carbon atoms.

For example, adjacent R₁₁ groups may be linked to each other to form a substituted or unsubstituted aryl group having 6 to 20 carbon atoms; a substituted or unsubstituted heterocycloalkyl group having 2 to 15 carbon atoms; or a substituted or unsubstituted heteroaryl group having 2 to 15 carbon atoms.

In exemplary embodiments, adjacent R₁₁ groups may be linked to each other to form a substituted or unsubstituted aryl group having 6 to 60 carbon atoms; a substituted or unsubstituted heterocycloalkyl group having 2 to 60 carbon atoms, or a substituted or unsubstituted heteroaryl group having 2 to 60 carbon atoms, each of which may be substituted or unsubstituted with deuterium.

For example, adjacent R₁₁ groups may be linked to each other to form a substituted or unsubstituted aryl group having 6 to 40 carbon atoms; a substituted or unsubstituted heterocycloalkyl group having 2 to 40 carbon atoms; or a substituted or unsubstituted heteroaryl group having 2 to 40 carbon atoms, each of which may be substituted or unsubstituted with deuterium.

For example, adjacent R₁₁ groups may be linked to each other to form a substituted or unsubstituted aryl group having 6 to 20 carbon atoms; a substituted or unsubstituted heterocycloalkyl group having 2 to 15 carbon atoms; or a substituted or unsubstituted heteroaryl group having 2 to 15 carbon atoms, each of which may be substituted or unsubstituted with deuterium.

In exemplary embodiments, when X₁, X₂, and X₄ are CR₁₁, and X₃ and X₅ are N, R₁₁ of X₁ and R₁₁ of X₂ may be linked to each other to form a ring

In exemplary embodiments, when X₂, X₃, and X₄ are CR₁₁, and X₁ and X₅ are N, R₁₁ of X₂ and R₁₁ of X₃, or R₁₁ of X₃ and R₁₁ of X₄ may be linked to each other to form a ring

In exemplary embodiments, when X₂, X₃, and X₅ are CR₁₁, and X₁ and X₄ are N, R₁₁ of X₁ and R₁₁ of X₂ may be linked to each other to form a ring.

In exemplary embodiments, when X₂, X₄, and X, are CR₁₁, and X₁ and X³ are N, Ru of X₄ and R₁₁ of Xₛ may be linked to each other to form a ring

According to exemplary embodiments, the N-Het may be represented by any one of Formulae 2-1 to 2-4 below.

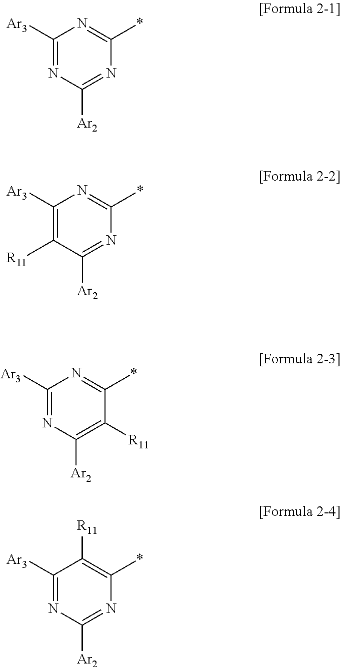

In Formulae 2-1 to 2-4, R₁₁ may have the same definition as described above.

In Formulae 2-1 to 2-4, Ar₂ and Ar₃ are each independently a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms.

According to some embodiments, Ar₂ and Ar₃ may each independently be a substituted or unsubstituted aryl group having 6 to 40 carbon atoms; a substituted or unsubstituted heterocycloalkyl group having 2 to 40 carbon atoms; or a substituted or unsubstituted heteroaryl group having 2 to 40 carbon atoms.

For example, Ar₂ and Ar₃ may each independently be a substituted or unsubstituted aryl group having 6 to 20 carbon atoms; a substituted or unsubstituted heterocycloalkyl group having 2 to 15 carbon atoms; or a substituted or unsubstituted heteroaryl group having 2 to 15 carbon atoms.

According to some embodiments, Ar₂ and Ar₃ may each independently be a substituted or unsubstituted aryl group having 6 to 40 carbon atoms; a substituted or unsubstituted heterocycloalkyl group having 2 to 40 carbon atoms; or a substituted or unsubstituted heteroaryl group having 2 to 40 carbon atoms, each of which may be substituted or unsubstituted with deuterium.

For example, Ar₂ and Ar₃ may each independently be a substituted or unsubstituted aryl group having 6 to 20 carbon atoms; a substituted or unsubstituted heterocycloalkyl group having 2 to 15 carbon atoms; or a substituted or unsubstituted heteroaryl group having 2 to 15 carbon atoms, each of which may be substituted or unsubstituted with deuterium.

For example, Ar₂ and Ar₃ may each independently be a substituted or unsubstituted phenyl group; a substituted or unsubstituted biphenyl group; a substituted or unsubstituted terphenyl group; a substituted or unsubstituted naphthyl group; a substituted or unsubstituted phenanthryl group; a substituted or unsubstituted triphenylenyl group; a substituted or unsubstituted anthracenyl group; a substituted or unsubstituted dimethylfluorenyl group; a substituted or unsubstituted diethylfluorenyl group; a substituted or unsubstituted spirobifluorenyl group; a substituted or unsubstituted furan group; a substituted or unsubstituted thiophenyl group; a substituted or unsubstituted benzofuran group; a substituted or unsubstituted benzothiophene group; a substituted or unsubstituted dibenzofuran group; a substituted or unsubstituted dibenzothiophene group; a substituted or unsubstituted carbazolyl group; or a combination thereof.

For example, Ar₂ and Ar₃ may each independently be a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a phenanthryl group substituted or unsubstituted with deuterium; a triphenylenyl group substituted or unsubstituted with deuterium; a dimethylfluorenyl group substituted or unsubstituted with deuterium; a diphenylfluorenyl group substituted or unsubstituted with deuterium; a spirobifluorenyl group substituted or unsubstituted with deuterium; a furan group substituted or unsubstituted with deuterium; a thiophene group substituted or unsubstituted with deuterium; a benzofuran group substituted or unsubstituted with deuterium; a benzothiophene group substituted or unsubstituted with deuterium; a dibenzofuran group substituted or unsubstituted with deuterium; a dibenzothiophene group substituted or unsubstituted with deuterium; an anthracenyl group substituted or unsubstituted with deuterium; a carbazolyl group substituted or unsubstituted with deuterium; or a combination thereof.

According to exemplary embodiments, the N-Het may be represented by any one of Formula 2-5-1 to 2-5-5, Formula 2-6-1 to 2-6-5, and Formula 2-7 below.

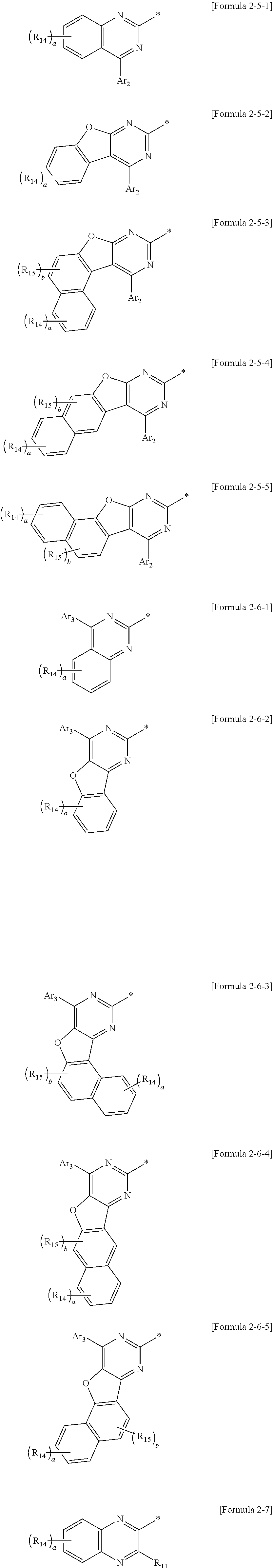

In Formulae 2-5-1 to 2-5-5, Formulae 2-6-1 to 2-6-5, and Formula 2-7, Ar, and Ar, may have the same definitions as described above.

In Formulae 2-5-1 to 2-5-5, Formulae 2-6-1 to 2-6-5, and Formula 2-7, Ru and Ris are each independently selected from the group consisting of hydrogen; deuterium; halogen; a substituted or unsubstituted alkyl group having 1 to 60 carbon atoms, a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups may be linked to form a substituted or unsubstituted 5- to 9-membered saturated or unsaturated ring

R, R′ and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In Formulae 2-5-1 to 2-5-5, Formulae 2-6-1 to 2-6-5, and Formula 2-7, a may be 0 or an integer from 1 to 4, and b may be 0, 1, or 2. For example, a may be 0 or 1, and b may be 0 or 1.

In Formulae 2-5-1 to 2-5-5, Formulae 2-6-1 to 2-6-5, and Formula 2-7, when a is an integer from 2 to 4, a plurality of R₁₄ groups may be the same or different.

In Formulae 2-5-3 to 2-5-5 and Formulae 2-6-3 to 2-6-5, when b is 2, a plurality of R₁₅ groups may be the same or different.

For example, when a is 4 and b is 2, a plurality of R₁₄ groups and R₁₅ groups may each independently be hydrogen or deuterium.

According to exemplary embodiments, the remainders of R₁ to R₄ that are not N-Het may be hydrogen or a substituent. For example, the remainders of R1 to R4 that are not N-Het may be hydrogen or deuterium.

In Formula 1, one of R₅ to R₁₀ is Ar₁. Ar₁ is a substituted or unsubstituted aryl group having 6 to 60 carbon atoms; a substituted or unsubstituted oxygen-containing heterocycloalkyl group having 2 to 60 carbon atoms; a substituted or unsubstituted sulfur-containing heterocycloalkyl group having 2 to 60 carbon atoms; a substituted or unsubstituted oxygen-containing heteroaryl group having 2 to 60 carbon atoms; or a substituted or unsubstituted sulfur-containing heteroaryl group having 2 to 60 carbon atoms.

According to exemplary embodiments, Ar₁ may be a substituted or unsubstituted aryl group having 6 to 40 carbon atoms; a substituted or unsubstituted oxygen-containing heterocycloalkyl group having 2 to 40 carbon atoms; a substituted or unsubstituted sulfur-containing heterocycloalkyl group having 2 to 40 carbon atoms; a substituted or unsubstituted oxygen-containing heteroaryl group having 2 to 40 carbon atoms; or a substituted or unsubstituted sulfur-containing heteroaryl group having 2 to 40 carbon atoms.

According to some embodiments, Ar₁ may be a substituted or unsubstituted aryl group having 6 to 20 carbon atoms; a substituted or unsubstituted oxygen-containing heterocycloalkyl group having 2 to 15 carbon atoms; a substituted or unsubstituted sulfur-containing heterocycloalkyl group having 2 to 15 carbon atoms; a substituted or unsubstituted oxygen-containing heteroaryl group having 2 to 15 carbon atoms; or a substituted or unsubstituted sulfur-containing heteroaryl group having 2 to 15 carbon atoms.

According to exemplary embodiments, Ar₁ may be a substituted or unsubstituted aryl group having 6 to 60 carbon atoms; a substituted or unsubstituted oxygen-containing heterocycloalkyl group having 2 to 60 carbon atoms; a substituted or unsubstituted sulfur-containing heterocycloalkyl group having 2 to 60 carbon atoms; a substituted or unsubstituted oxygen-containing heteroaryl group having 2 to 60 carbon atoms; or a substituted or unsubstituted sulfur-containing heteroaryl group having 2 to 60 carbon atoms, each of which may be substituted or unsubstituted with deuterium.

According to some embodiments, Ar₁ may be a substituted or unsubstituted aryl group having 6 to 40 carbon atoms; a substituted or unsubstituted oxygen-containing heterocycloalkyl group having 2 to 40 carbon atoms; a substituted or unsubstituted sulfur-containing heterocycloalkyl group having 2 to 40 carbon atoms; a substituted or unsubstituted oxygen-containing heteroaryl group having 2 to 40 carbon atoms; or a substituted or unsubstituted sulfur-containing heteroaryl group having 2 to 40 carbon atoms, each of which may be substituted or unsubstituted with deuterium.

For example, Ar₁ may be a substituted or unsubstituted aryl group having 6 to 20 carbon atoms; a substituted or unsubstituted oxygen-containing heterocycloalkyl group having 2 to 15 carbon atoms; a substituted or unsubstituted sulfur-containing heterocycloalkyl group having 2 to 15 carbon atoms; a substituted or unsubstituted oxygen-containing heteroaryl group having 2 to 15 carbon atoms; or a substituted or unsubstituted sulfur-containing heteroaryl group having 2 to 15 carbon atoms, each of which may be substituted or unsubstituted with deuterium.

In some embodiments, Ar₁ may not include nitrogen.

When Ar₁ is a substituted or unsubstituted oxygen-containing heterocycloalkyl group having 2 to 60 carbon atoms; a substituted or unsubstituted sulfur-containing heterocycloalkyl group having 2 to 60 carbon atoms; a substituted or unsubstituted oxygen-containing heteroaryl group having 2 to 60 carbon atoms; or a substituted or unsubstituted sulfur-containing heteroaryl group having 2 to 60 carbon atoms, the N-Het may not include oxygen or sulfur.

According to exemplary embodiments, when Ar₁ includes an oxygen- or sulfur-containing heterocycle, N-Het may not include oxygen or sulfur.

The remainders of R₅ to R₁₀ that are not Ar₁ are independently hydrogen or a substituent. For example, the remainders of R₅ to R₁₀ that are not Ar₁ may each independently be hydrogen or deuterium.

For example, Ar₁ may be a substituted or unsubstituted phenyl group; a substituted or unsubstituted biphenyl group; a substituted or unsubstituted terphenyl group; a substituted or unsubstituted naphthyl group; a substituted or unsubstituted phenanthryl group; a substituted or unsubstituted triphenylenyl group; a substituted or unsubstituted anthracenyl group; a substituted or unsubstituted dimethylfluorenyl group; a substituted or unsubstituted diethylfluorenyl group; a substituted or unsubstituted spirobifluorenyl group; a substituted or unsubstituted furan group; a substituted or unsubstituted thiophenyl group; a substituted or unsubstituted benzofuran group; a substituted or unsubstituted benzothiophene group; a substituted or unsubstituted dibenzofuran group; a substituted or unsubstituted dibenzothiophene group; a substituted or unsubstituted N-carbazolyl group; a substituted or unsubstituted C-carbazolyl group; or a combination thereof.

For example, Ar₁ may be a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a phenanthryl group substituted or unsubstituted with deuterium; a triphenylenyl group substituted or unsubstituted with deuterium; a dimethylfluorenyl group substituted or unsubstituted with deuterium; a diphenylfluorenyl group substituted or unsubstituted with deuterium; a spirobifluorenyl group substituted or unsubstituted with deuterium; a furan group substituted or unsubstituted with deuterium; a thiophene group substituted or unsubstituted with deuterium; a benzofuran group substituted or unsubstituted with deuterium; a benzothiophene group substituted or unsubstituted with deuterium; a dibenzofuran group substituted or unsubstituted with deuterium; a dibenzothiophene group substituted or unsubstituted with deuterium; an anthracenyl group substituted or unsubstituted with deuterium; an N-carbazolyl group substituted or unsubstituted with deuterium; a C-carbazolyl group substituted or unsubstituted with deuterium; or a combination thereof.

According to exemplary embodiments, the heterocyclic compound may be represented by any one of Formulae 1-1 to 1-2 below.

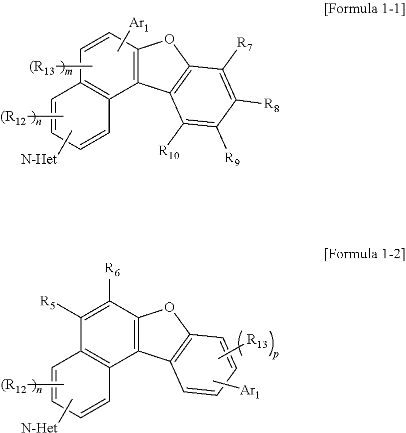

In Formulae 1-1 and 1-2, R₅ to R₁₀, N-Het, and Ar₁ may have the same definitions as described above.

R₁₂ and R₁₃ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(—O)RR′, or adjacent groups may be linked to form a substituted or unsubstituted 5- to 9-membered saturated or unsaturated ring.

R, R′ and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

For example, R₁₂ and R₁₃ may each independently be hydrogen or deuterium.

In Formulae 1-1 and 1-2, n and p may each independently be 0 or an integer from 1 to 3, and m may be 0 or 1. For example, n, m, and p may each independently be 0 or 1.

According to exemplary embodiments, when n is 2 or 3, a plurality of R₁₂ groups may be the same or different.

According to exemplary embodiments, when p is 2 or 3, a plurality of R₁₃ groups may be the same or different.

According to exemplary embodiments, the deuterium content of the compound of Formula 1 may be 0%, or greater than 0 and not more than 100%.

According to exemplary embodiments, the deuterium content of the compound of Formula 1 may be 0%, or 5% to 100%.

According to exemplary embodiments, the deuterium content of the compound of Formula 1 may be 0%, or 10% to 100%.

According to exemplary embodiments, the deuterium content of the compound of Formula 1 may be 0%, or 15% to 100%.

According to exemplary embodiments, the deuterium content of the compound of Formula 1 may be 0%, or 20% to 100%.

According to exemplary embodiments, the deuterium content of the compound of Formula 1 may be 0%, or 25% to 100%.

According to exemplary embodiments, the deuterium content of the compound of Formula 1 may be 0%, or 30% to 100%.

According to exemplary embodiments, the deuterium content of the compound of Formula 1 may be 0%, or 50% to 100%.

According to exemplary embodiments, the deuterium content of the compound of Formula 1 may be 0%, or 70% to 100%.

According to exemplary embodiments, the deuterium content of the compound of Formula 1 may be 0%, or 90% to 100%.

According to exemplary embodiments, the deuterium content of the compound of Formula 1 may be 0%.

According to exemplary embodiments, the deuterium content of the compound of Formula 1 may be 30% to 100%.

According to exemplary embodiments, the deuterium content of the compound of Formula 1 may be 50% to 100%.

According to exemplary embodiments, the deuterium content of the compound of Formula 1 may be 70% to 100%.

According to exemplary embodiments, the deuterium content of the compound of Formula 1 may be 90% to 100%.

The heterocyclic compound may be represented by any one of the formulae below.

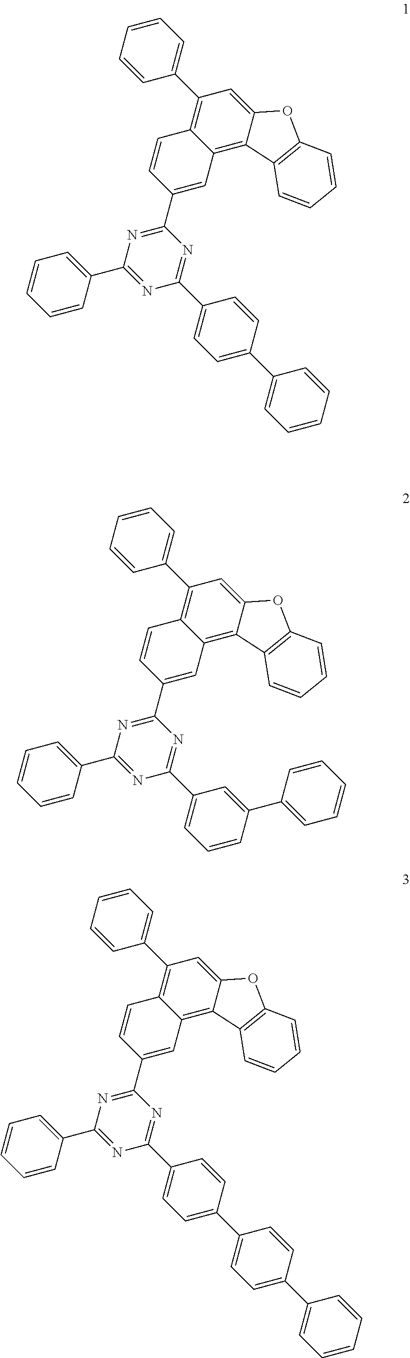 [SMILES: c1ccc(-c2ccc(-c3nc(-c4ccccc4)nc(-c4ccc5c(-c6ccccc6)cc6oc7ccccc7c6c5c4)n3)cc2)cc1 ; c1ccc(-c2cccc(-c3nc(-c4ccccc4)nc(-c4ccc5c(-c6ccccc6)cc6oc7ccccc7c6c5c4)n3)c2)cc1 ; c1ccc(-c2ccc(-c3ccc(-c4nc(-c5ccccc5)nc(-c5ccc6c(-c7ccccc7)cc7oc8ccccc8c7c6c5)n4)cc3)cc2)cc1]

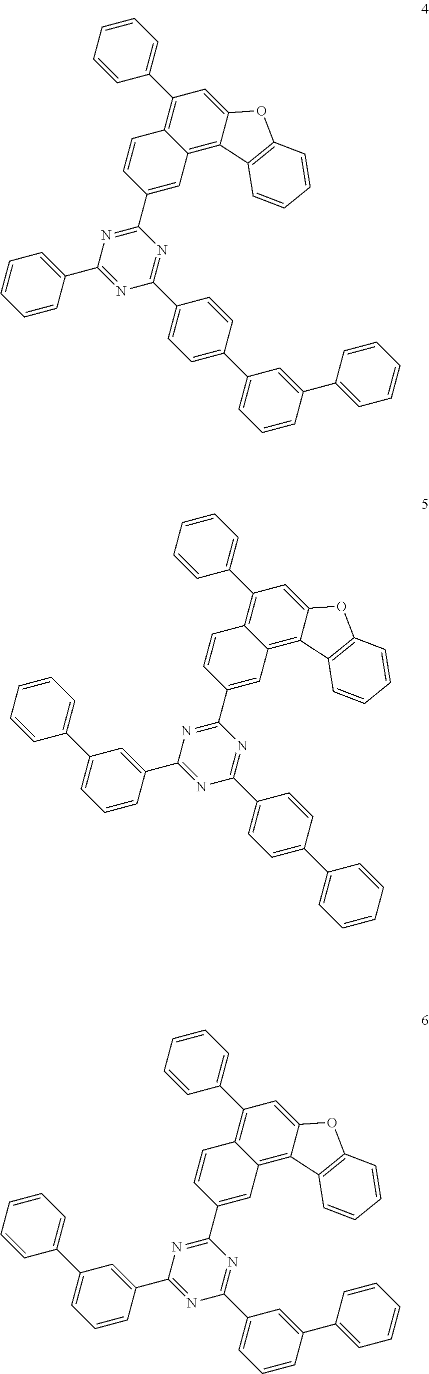 [SMILES: c1ccc(-c2cccc(-c3ccc(-c4nc(-c5ccccc5)nc(-c5ccc6c(-c7ccccc7)cc7oc8ccccc8c7c6c5)n4)cc3)c2)cc1 ; c1ccc(-c2ccc(-c3nc(-c4cccc(-c5ccccc5)c4)nc(-c4ccc5c(-c6ccccc6)cc6oc7ccccc7c6c5c4)n3)cc2)cc1 ; c1ccc(-c2cccc(-c3nc(-c4cccc(-c5ccccc5)c4)nc(-c4ccc5c(-c6ccccc6)cc6oc7ccccc7c6c5c4)n3)c2)cc1]

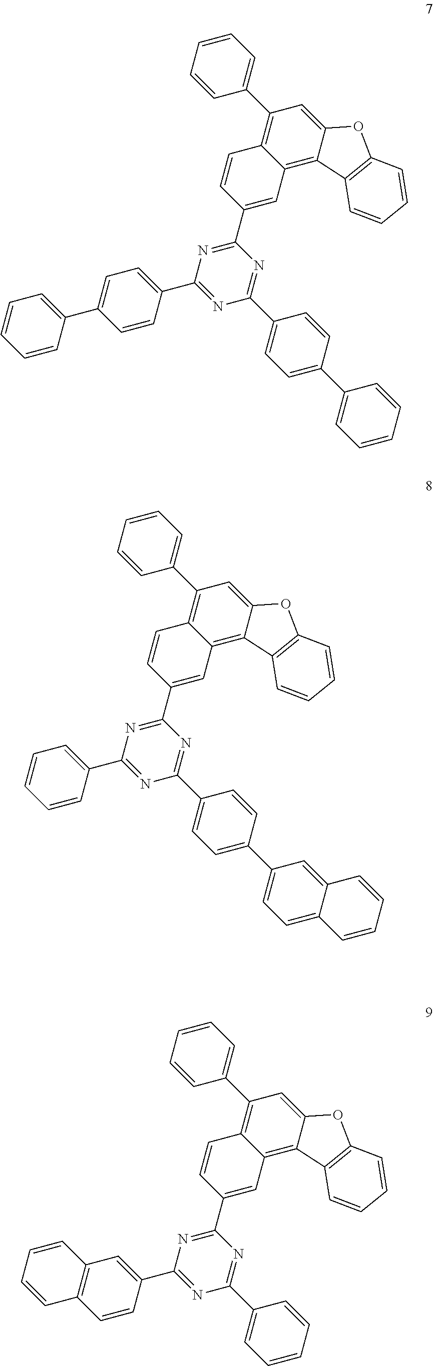 [SMILES: c1ccc(-c2ccc(-c3nc(-c4ccc(-c5ccccc5)cc4)nc(-c4ccc5c(-c6ccccc6)cc6oc7ccccc7c6c5c4)n3)cc2)cc1 ; c1ccc(-c2nc(-c3ccc(-c4ccc5ccccc5c4)cc3)nc(-c3ccc4c(-c5ccccc5)cc5oc6ccccc6c5c4c3)n2)cc1 ; c1ccc(-c2nc(-c3ccc4ccccc4c3)nc(-c3ccc4c(-c5ccccc5)cc5oc6ccccc6c5c4c3)n2)cc1]

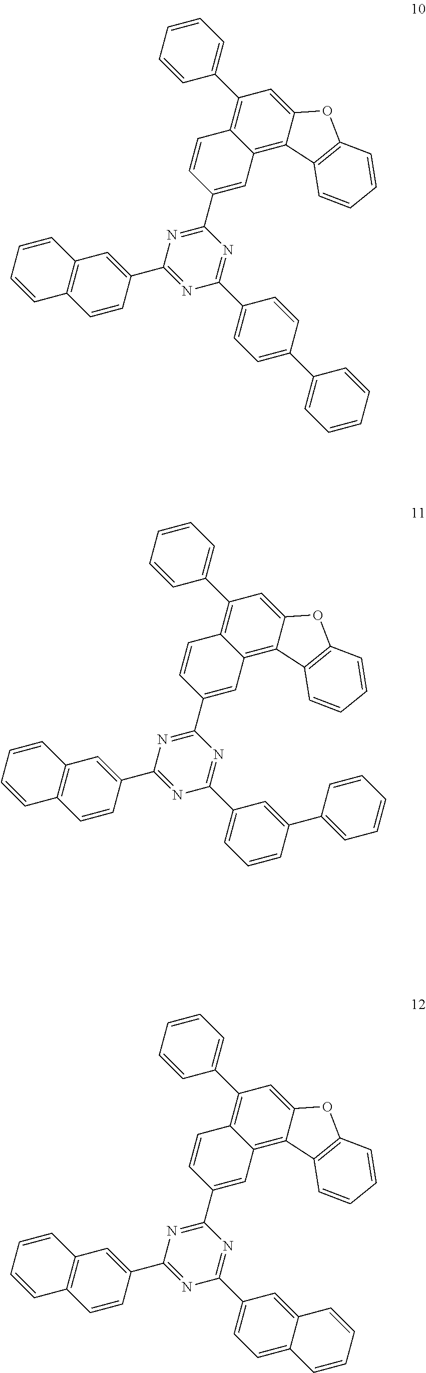 [SMILES: c1ccc(-c2ccc(-c3nc(-c4ccc5ccccc5c4)nc(-c4ccc5c(-c6ccccc6)cc6oc7ccccc7c6c5c4)n3)cc2)cc1 ; c1ccc(-c2cccc(-c3nc(-c4ccc5ccccc5c4)nc(-c4ccc5c(-c6ccccc6)cc6oc7ccccc7c6c5c4)n3)c2)cc1 ; c1ccc(-c2cc3oc4ccccc4c3c3cc(-c4nc(-c5ccc6ccccc6c5)nc(-c5ccc6ccccc6c5)n4)ccc23)cc1]

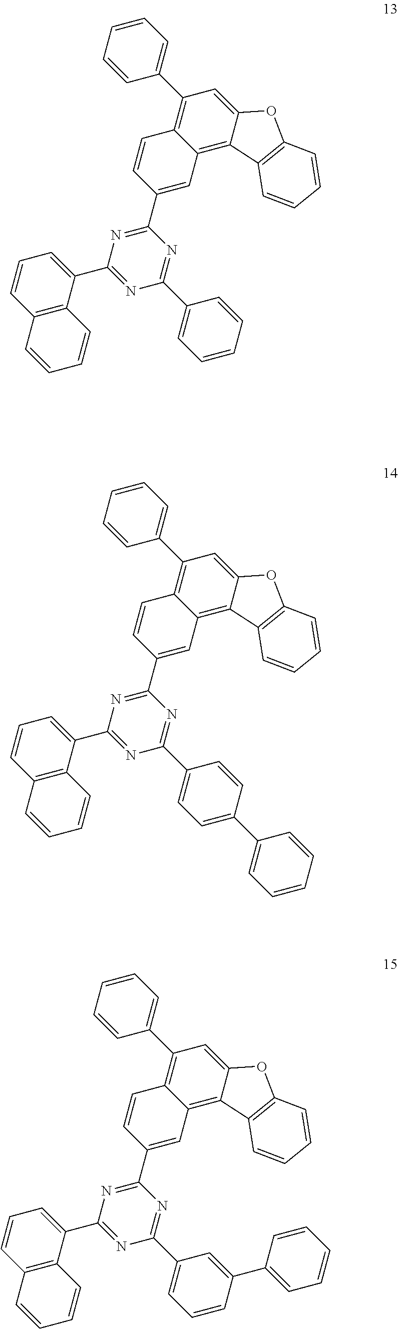 [SMILES: c1ccc(-c2nc(-c3ccc4c(-c5ccccc5)cc5oc6ccccc6c5c4c3)nc(-c3cccc4ccccc34)n2)cc1 ; c1ccc(-c2ccc(-c3nc(-c4ccc5c(-c6ccccc6)cc6oc7ccccc7c6c5c4)nc(-c4cccc5ccccc45)n3)cc2)cc1 ; c1ccc(-c2cccc(-c3nc(-c4ccc5c(-c6ccccc6)cc6oc7ccccc7c6c5c4)nc(-c4cccc5ccccc45)n3)c2)cc1]

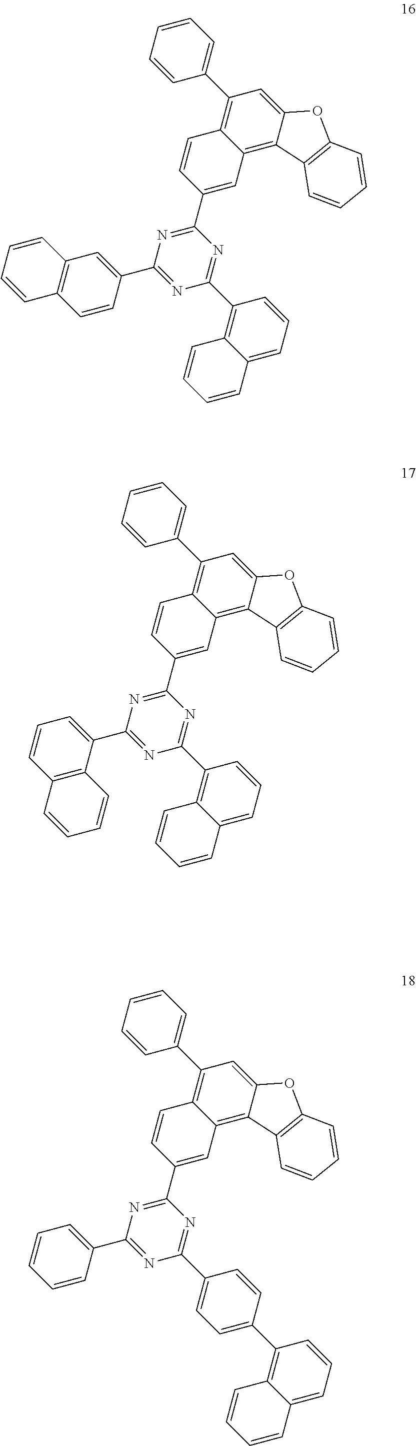 [SMILES: c1ccc(-c2cc3oc4ccccc4c3c3cc(-c4nc(-c5ccc6ccccc6c5)nc(-c5cccc6ccccc56)n4)ccc23)cc1 ; c1ccc(-c2cc3oc4ccccc4c3c3cc(-c4nc(-c5cccc6ccccc56)nc(-c5cccc6ccccc56)n4)ccc23)cc1 ; c1ccc(-c2nc(-c3ccc(-c4cccc5ccccc45)cc3)nc(-c3ccc4c(-c5ccccc5)cc5oc6ccccc6c5c4c3)n2)cc1]

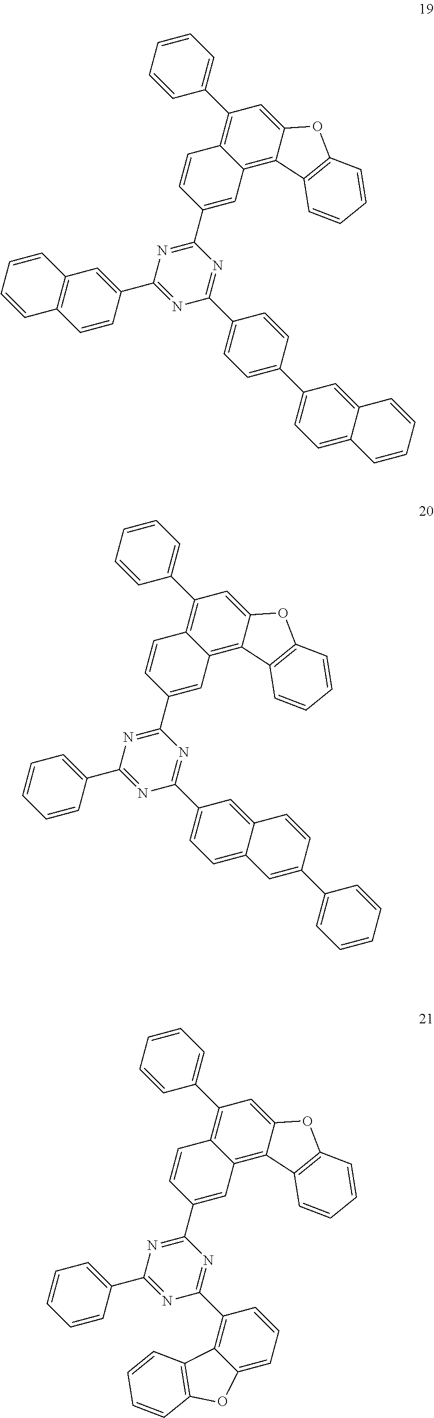 [SMILES: c1ccc(-c2cc3oc4ccccc4c3c3cc(-c4nc(-c5ccc(-c6ccc7ccccc7c6)cc5)nc(-c5ccc6ccccc6c5)n4)ccc23)cc1 ; c1ccc(-c2ccc3cc(-c4nc(-c5ccccc5)nc(-c5ccc6c(-c7ccccc7)cc7oc8ccccc8c7c6c5)n4)ccc3c2)cc1 ; c1ccc(-c2nc(-c3ccc4c(-c5ccccc5)cc5oc6ccccc6c5c4c3)nc(-c3cccc4oc5ccccc5c34)n2)cc1]

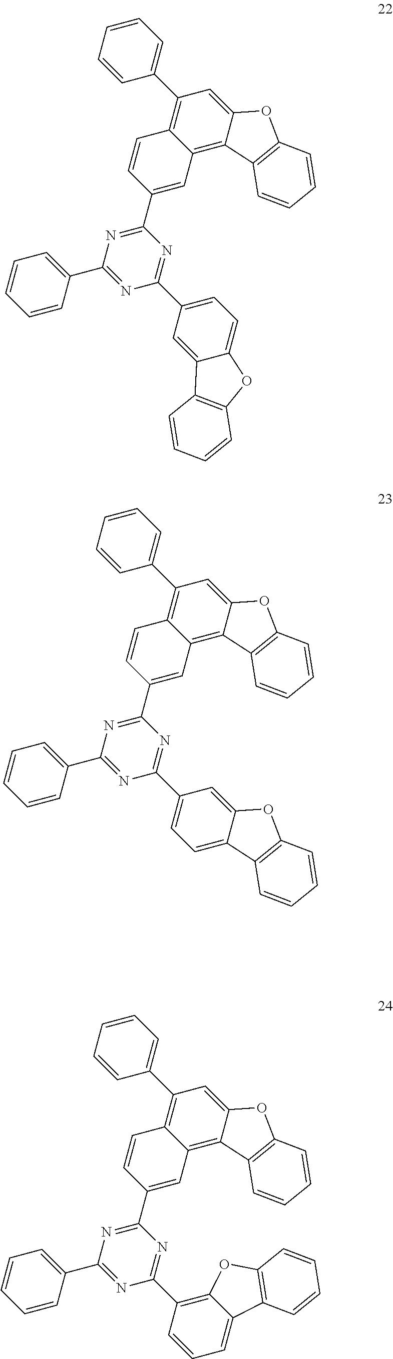 [SMILES: c1ccc(-c2nc(-c3ccc4oc5ccccc5c4c3)nc(-c3ccc4c(-c5ccccc5)cc5oc6ccccc6c5c4c3)n2)cc1 ; c1ccc(-c2nc(-c3ccc4c(c3)oc3ccccc34)nc(-c3ccc4c(-c5ccccc5)cc5oc6ccccc6c5c4c3)n2)cc1 ; c1ccc(-c2nc(-c3ccc4c(-c5ccccc5)cc5oc6ccccc6c5c4c3)nc(-c3cccc4c3oc3ccccc34)n2)cc1]

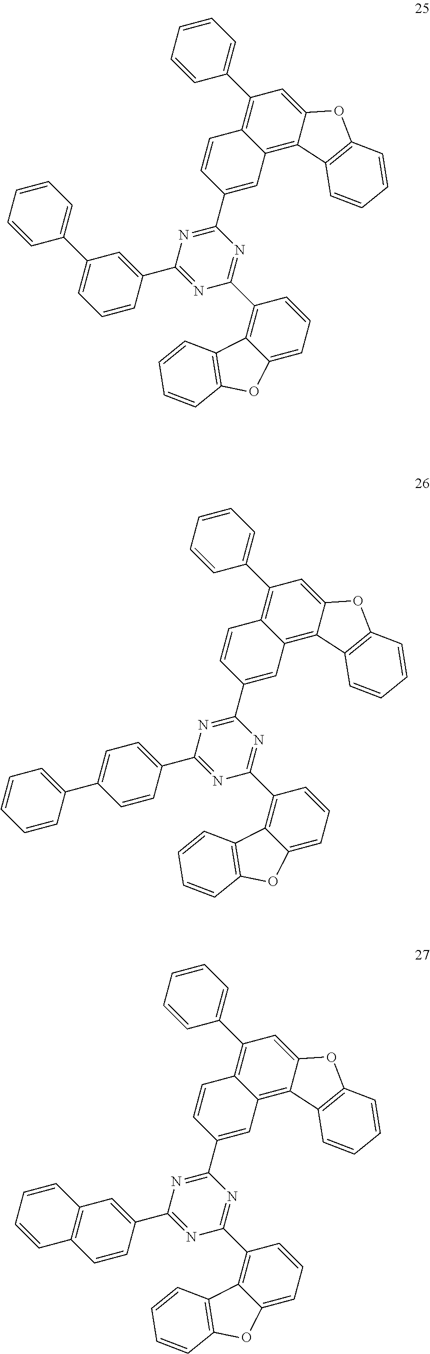 [SMILES: c1ccc(-c2cccc(-c3nc(-c4ccc5c(-c6ccccc6)cc6oc7ccccc7c6c5c4)nc(-c4cccc5oc6ccccc6c45)n3)c2)cc1 ; c1ccc(-c2ccc(-c3nc(-c4ccc5c(-c6ccccc6)cc6oc7ccccc7c6c5c4)nc(-c4cccc5oc6ccccc6c45)n3)cc2)cc1 ; c1ccc(-c2cc3oc4ccccc4c3c3cc(-c4nc(-c5ccc6ccccc6c5)nc(-c5cccc6oc7ccccc7c56)n4)ccc23)cc1]

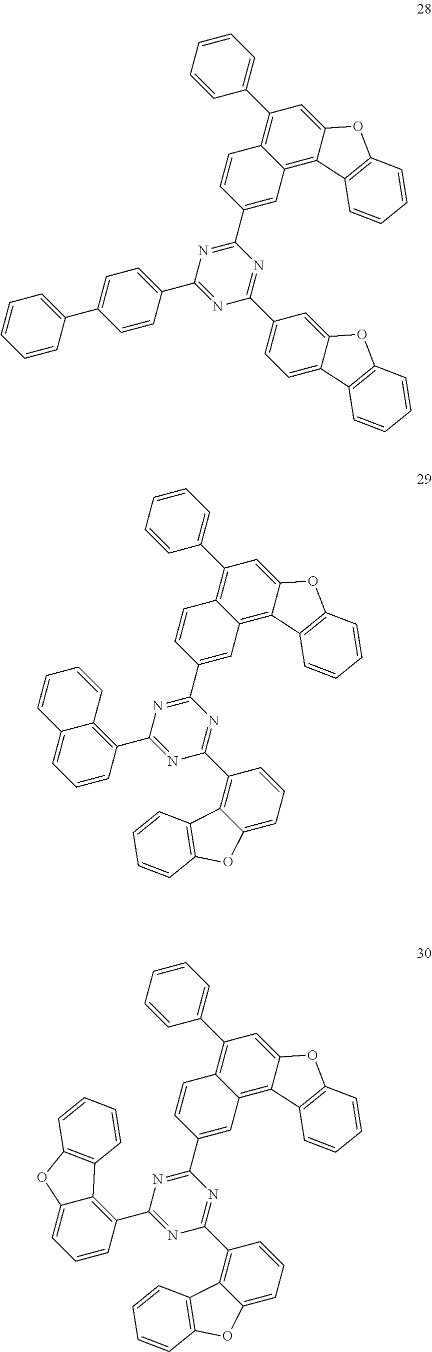 [SMILES: c1ccc(-c2ccc(-c3nc(-c4ccc5c(c4)oc4ccccc45)nc(-c4ccc5c(-c6ccccc6)cc6oc7ccccc7c6c5c4)n3)cc2)cc1 ; c1ccc(-c2cc3oc4ccccc4c3c3cc(-c4nc(-c5cccc6ccccc56)nc(-c5cccc6oc7ccccc7c56)n4)ccc23)cc1 ; c1ccc(-c2cc3oc4ccccc4c3c3cc(-c4nc(-c5cccc6oc7ccccc7c56)nc(-c5cccc6oc7ccccc7c56)n4)ccc23)cc1]

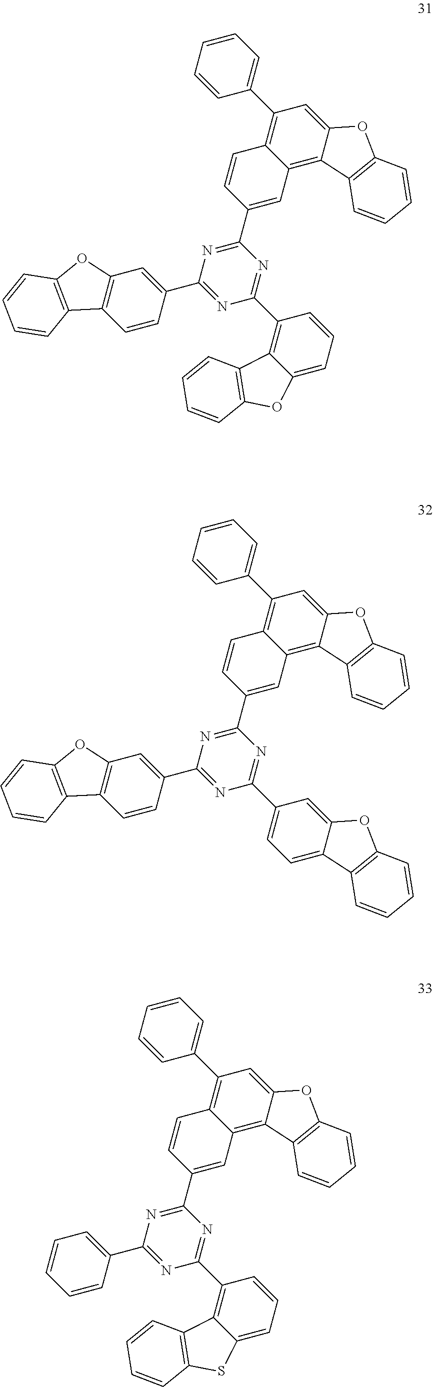 [SMILES: c1ccc(-c2cc3oc4ccccc4c3c3cc(-c4nc(-c5ccc6c(c5)oc5ccccc56)nc(-c5cccc6oc7ccccc7c56)n4)ccc23)cc1 ; c1ccc(-c2cc3oc4ccccc4c3c3cc(-c4nc(-c5ccc6c(c5)oc5ccccc56)nc(-c5ccc6c(c5)oc5ccccc56)n4)ccc23)cc1 ; c1ccc(-c2nc(-c3ccc4c(-c5ccccc5)cc5oc6ccccc6c5c4c3)nc(-c3cccc4sc5ccccc5c34)n2)cc1]

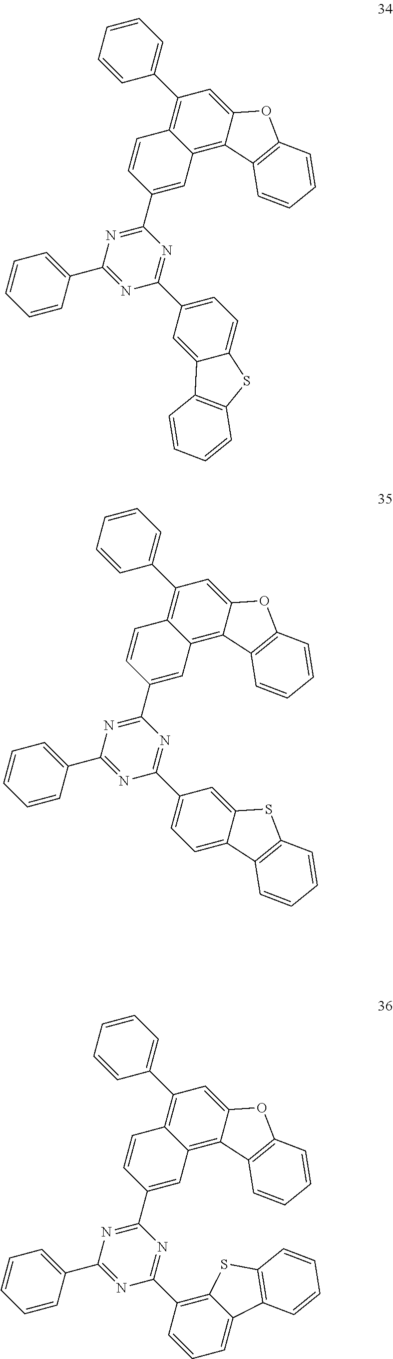 [SMILES: c1ccc(-c2nc(-c3ccc4sc5ccccc5c4c3)nc(-c3ccc4c(-c5ccccc5)cc5oc6ccccc6c5c4c3)n2)cc1 ; c1ccc(-c2nc(-c3ccc4c(c3)sc3ccccc34)nc(-c3ccc4c(-c5ccccc5)cc5oc6ccccc6c5c4c3)n2)cc1 ; c1ccc(-c2nc(-c3ccc4c(-c5ccccc5)cc5oc6ccccc6c5c4c3)nc(-c3cccc4c3sc3ccccc34)n2)cc1]

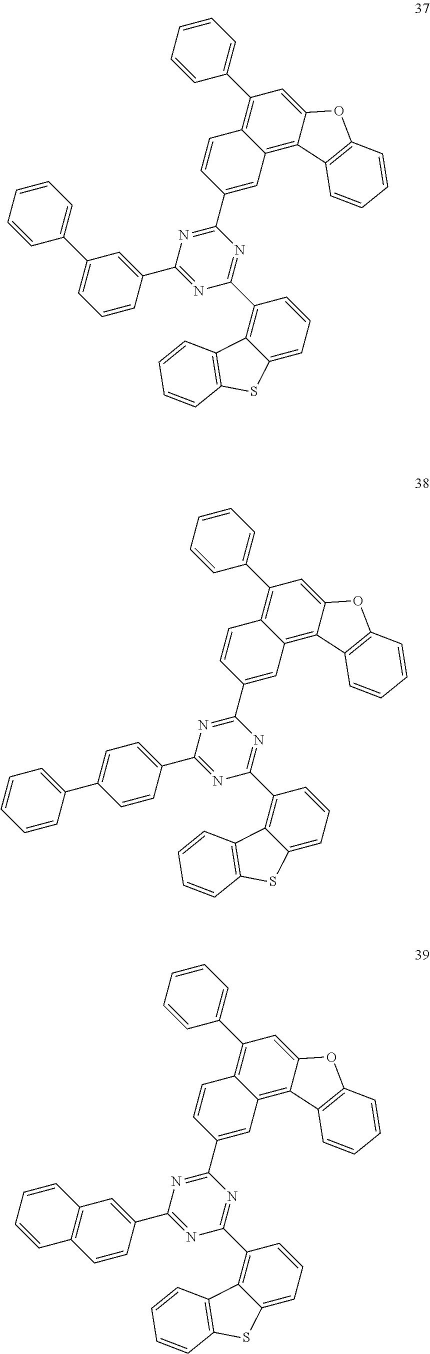 [SMILES: c1ccc(-c2cccc(-c3nc(-c4ccc5c(-c6ccccc6)cc6oc7ccccc7c6c5c4)nc(-c4cccc5sc6ccccc6c45)n3)c2)cc1 ; c1ccc(-c2ccc(-c3nc(-c4ccc5c(-c6ccccc6)cc6oc7ccccc7c6c5c4)nc(-c4cccc5sc6ccccc6c45)n3)cc2)cc1 ; c1ccc(-c2cc3oc4ccccc4c3c3cc(-c4nc(-c5ccc6ccccc6c5)nc(-c5cccc6sc7ccccc7c56)n4)ccc23)cc1]

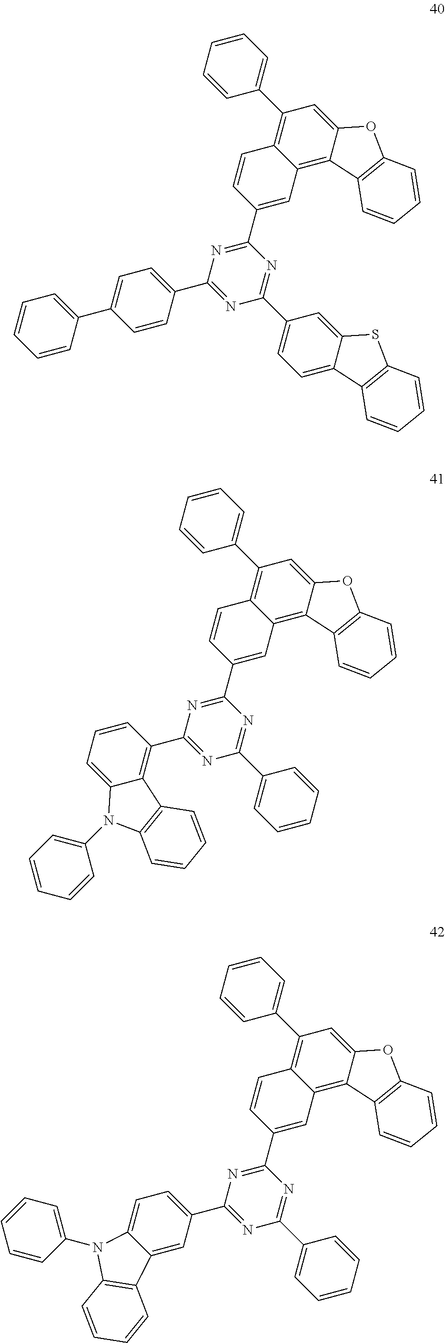 [SMILES: c1ccc(-c2ccc(-c3nc(-c4ccc5c(c4)sc4ccccc45)nc(-c4ccc5c(-c6ccccc6)cc6oc7ccccc7c6c5c4)n3)cc2)cc1 ; c1ccc(-c2nc(-c3ccc4c(-c5ccccc5)cc5oc6ccccc6c5c4c3)nc(-c3cccc4c3c3ccccc3n4-c3ccccc3)n2)cc1 ; c1ccc(-c2nc(-c3ccc4c(-c5ccccc5)cc5oc6ccccc6c5c4c3)nc(-c3ccc4c(c3)c3ccccc3n4-c3ccccc3)n2)cc1]

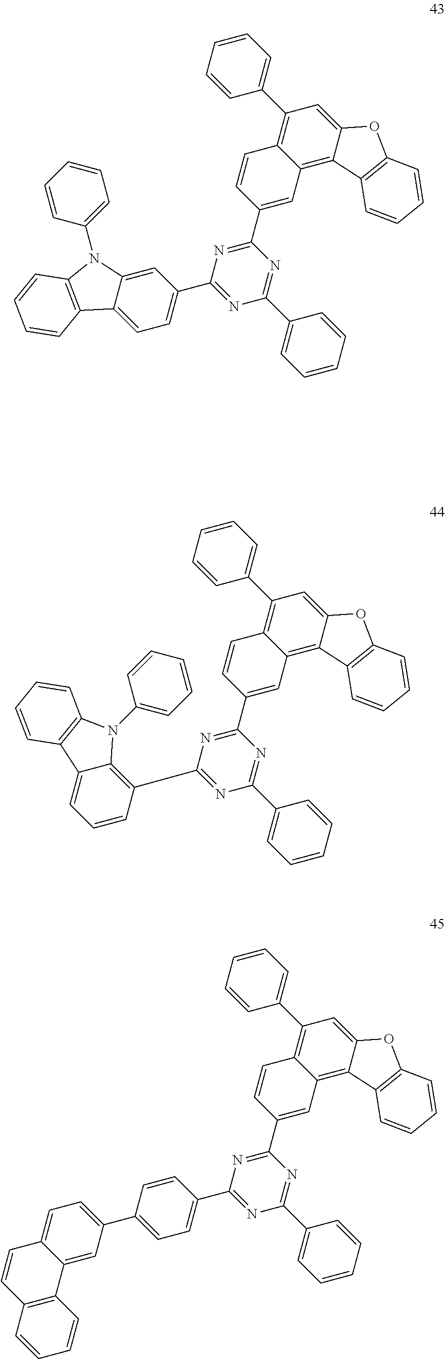 [SMILES: c1ccc(-c2nc(-c3ccc4c(-c5ccccc5)cc5oc6ccccc6c5c4c3)nc(-c3ccc4c5ccccc5n(-c5ccccc5)c4c3)n2)cc1 ; c1ccc(-c2nc(-c3ccc4c(-c5ccccc5)cc5oc6ccccc6c5c4c3)nc(-c3cccc4c5ccccc5n(-c5ccccc5)c34)n2)cc1 ; c1ccc(-c2nc(-c3ccc(-c4ccc5ccc6ccccc6c5c4)cc3)nc(-c3ccc4c(-c5ccccc5)cc5oc6ccccc6c5c4c3)n2)cc1]

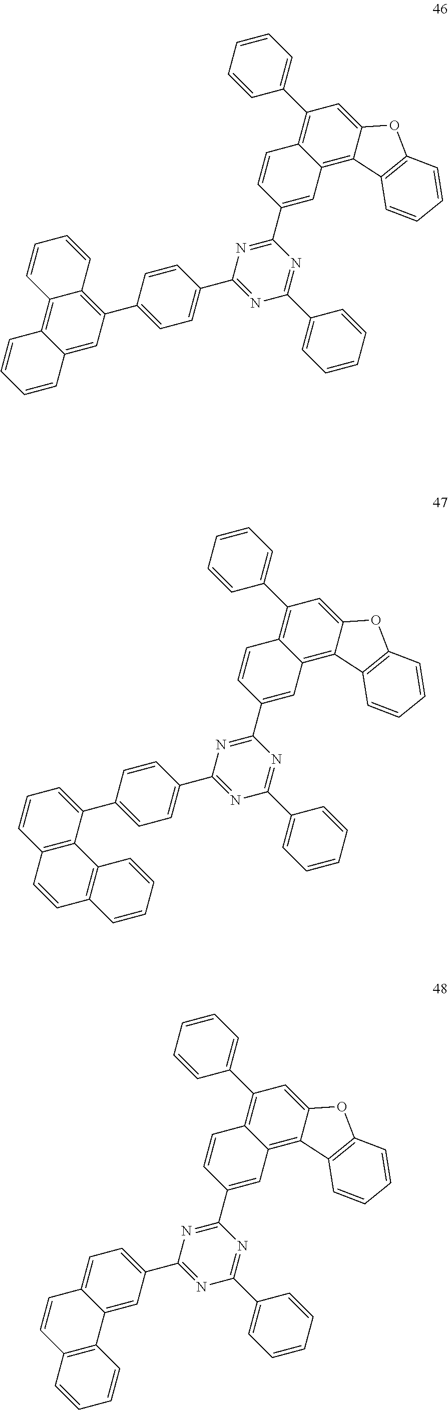 [SMILES: c1ccc(-c2nc(-c3ccc(-c4cc5ccccc5c5ccccc45)cc3)nc(-c3ccc4c(-c5ccccc5)cc5oc6ccccc6c5c4c3)n2)cc1 ; c1ccc(-c2nc(-c3ccc(-c4cccc5ccc6ccccc6c45)cc3)nc(-c3ccc4c(-c5ccccc5)cc5oc6ccccc6c5c4c3)n2)cc1 ; c1ccc(-c2nc(-c3ccc4ccc5ccccc5c4c3)nc(-c3ccc4c(-c5ccccc5)cc5oc6ccccc6c5c4c3)n2)cc1]

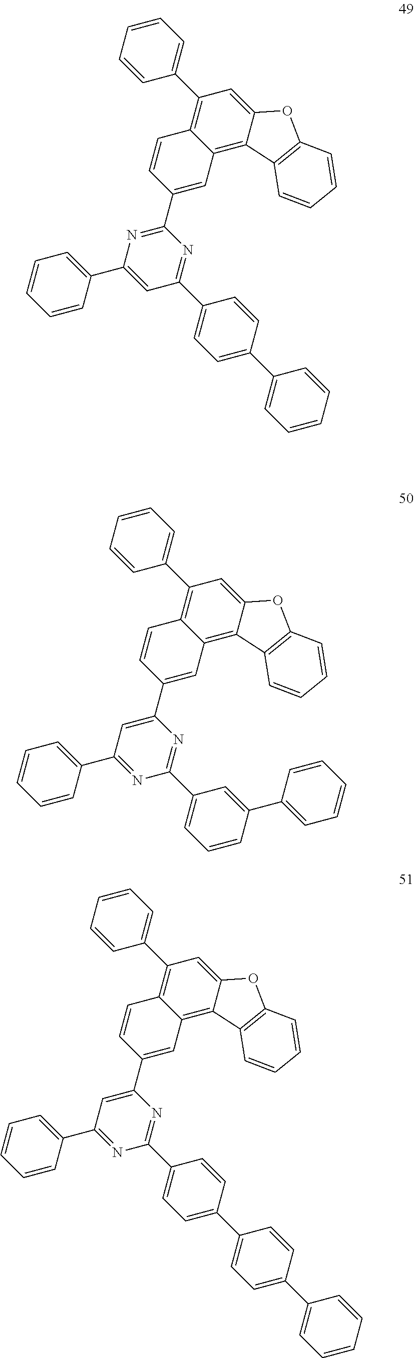 [SMILES: c1ccc(-c2ccc(-c3cc(-c4ccccc4)nc(-c4ccc5c(-c6ccccc6)cc6oc7ccccc7c6c5c4)n3)cc2)cc1 ; c1ccc(-c2cccc(-c3nc(-c4ccccc4)cc(-c4ccc5c(-c6ccccc6)cc6oc7ccccc7c6c5c4)n3)c2)cc1 ; c1ccc(-c2ccc(-c3ccc(-c4nc(-c5ccccc5)cc(-c5ccc6c(-c7ccccc7)cc7oc8ccccc8c7c6c5)n4)cc3)cc2)cc1]

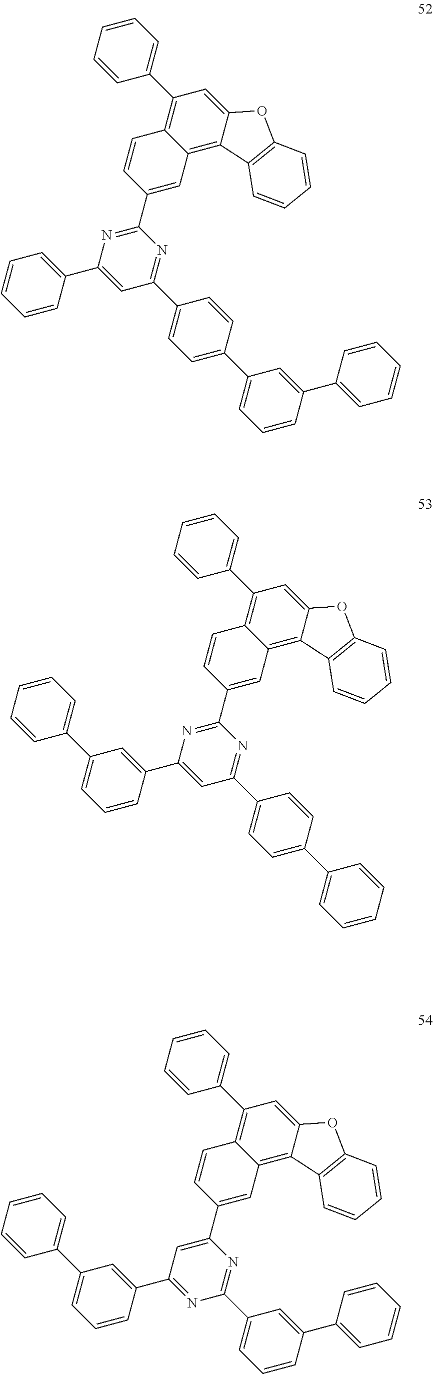 [SMILES: c1ccc(-c2cccc(-c3ccc(-c4cc(-c5ccccc5)nc(-c5ccc6c(-c7ccccc7)cc7oc8ccccc8c7c6c5)n4)cc3)c2)cc1 ; c1ccc(-c2ccc(-c3cc(-c4cccc(-c5ccccc5)c4)nc(-c4ccc5c(-c6ccccc6)cc6oc7ccccc7c6c5c4)n3)cc2)cc1 ; c1ccc(-c2cccc(-c3cc(-c4ccc5c(-c6ccccc6)cc6oc7ccccc7c6c5c4)nc(-c4cccc(-c5ccccc5)c4)n3)c2)cc1]

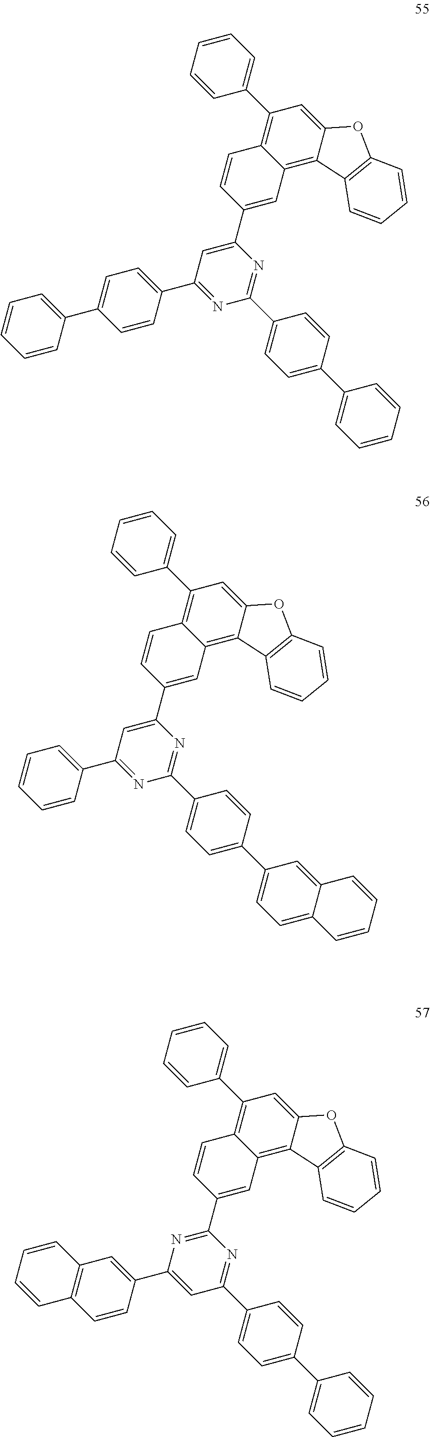 [SMILES: c1ccc(-c2ccc(-c3cc(-c4ccc5c(-c6ccccc6)cc6oc7ccccc7c6c5c4)nc(-c4ccc(-c5ccccc5)cc4)n3)cc2)cc1 ; c1ccc(-c2cc(-c3ccc4c(-c5ccccc5)cc5oc6ccccc6c5c4c3)nc(-c3ccc(-c4ccc5ccccc5c4)cc3)n2)cc1 ; c1ccc(-c2ccc(-c3cc(-c4ccc5ccccc5c4)nc(-c4ccc5c(-c6ccccc6)cc6oc7ccccc7c6c5c4)n3)cc2)cc1]

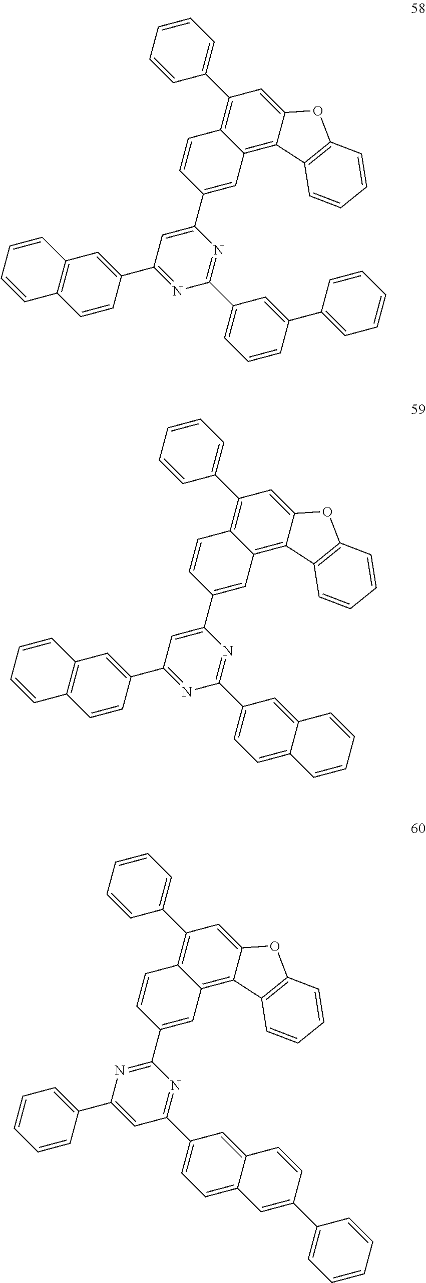 [SMILES: c1ccc(-c2cccc(-c3nc(-c4ccc5ccccc5c4)cc(-c4ccc5c(-c6ccccc6)cc6oc7ccccc7c6c5c4)n3)c2)cc1 ; c1ccc(-c2cc3oc4ccccc4c3c3cc(-c4cc(-c5ccc6ccccc6c5)nc(-c5ccc6ccccc6c5)n4)ccc23)cc1 ; c1ccc(-c2ccc3cc(-c4cc(-c5ccccc5)nc(-c5ccc6c(-c7ccccc7)cc7oc8ccccc8c7c6c5)n4)ccc3c2)cc1]

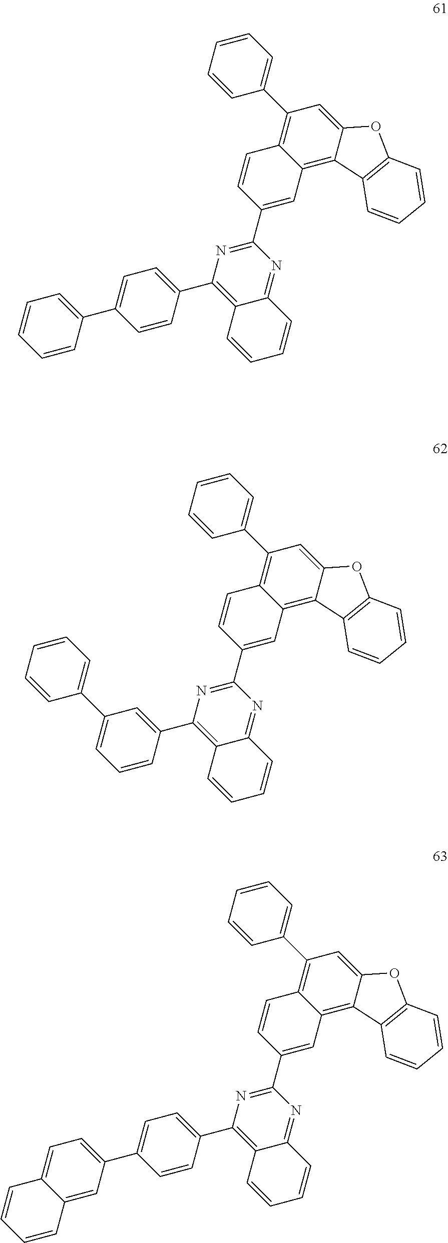 [SMILES: c1ccc(-c2ccc(-c3nc(-c4ccc5c(-c6ccccc6)cc6oc7ccccc7c6c5c4)nc4ccccc34)cc2)cc1 ; c1ccc(-c2cccc(-c3nc(-c4ccc5c(-c6ccccc6)cc6oc7ccccc7c6c5c4)nc4ccccc34)c2)cc1 ; c1ccc(-c2cc3oc4ccccc4c3c3cc(-c4nc(-c5ccc(-c6ccc7ccccc7c6)cc5)c5ccccc5n4)ccc23)cc1]

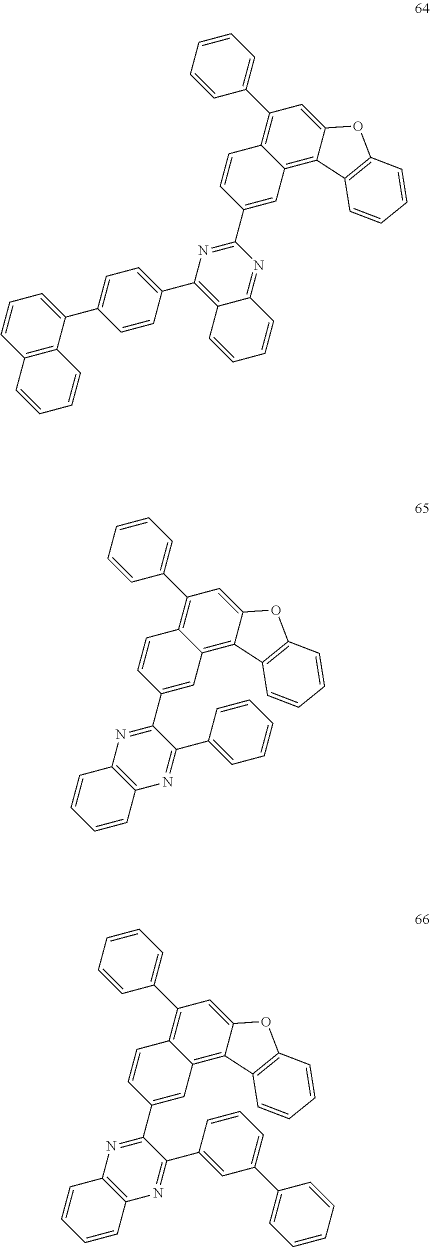 [SMILES: c1ccc(-c2cc3oc4ccccc4c3c3cc(-c4nc(-c5ccc(-c6cccc7ccccc67)cc5)c5ccccc5n4)ccc23)cc1 ; c1ccc(-c2nc3ccccc3nc2-c2ccc3c(-c4ccccc4)cc4oc5ccccc5c4c3c2)cc1 ; c1ccc(-c2cccc(-c3nc4ccccc4nc3-c3ccc4c(-c5ccccc5)cc5oc6ccccc6c5c4c3)c2)cc1]

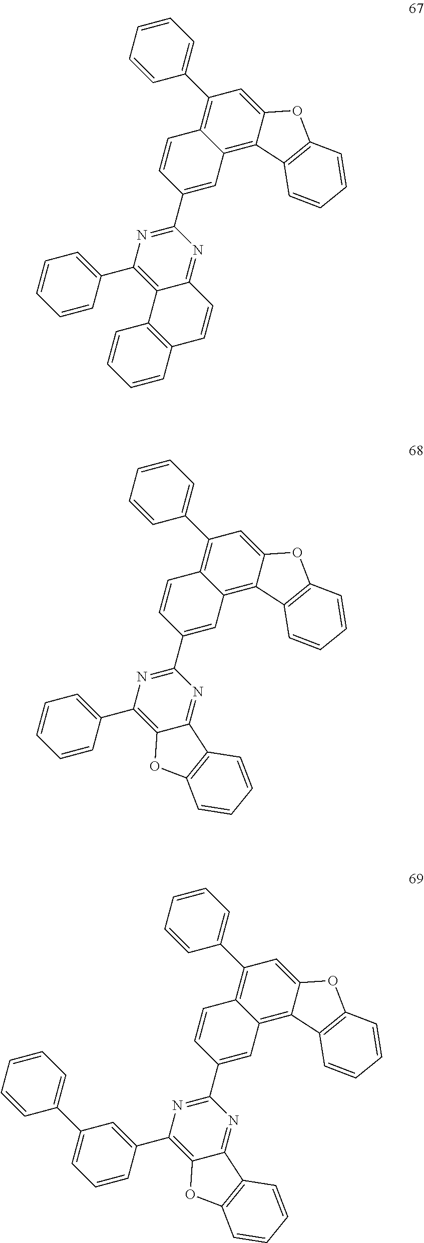 [SMILES: c1ccc(-c2cc3oc4ccccc4c3c3cc(-c4nc(-c5ccccc5)c5c(ccc6ccccc65)n4)ccc23)cc1 ; c1ccc(-c2cc3oc4ccccc4c3c3cc(-c4nc(-c5ccccc5)c5oc6ccccc6c5n4)ccc23)cc1 ; c1ccc(-c2cccc(-c3nc(-c4ccc5c(-c6ccccc6)cc6oc7ccccc7c6c5c4)nc4c3oc3ccccc34)c2)cc1]

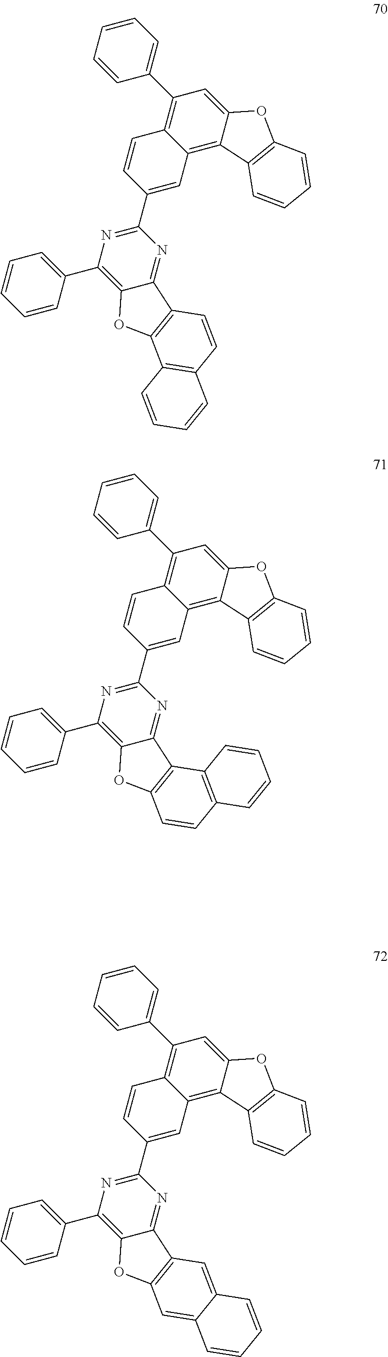 [SMILES: c1ccc(-c2cc3oc4ccccc4c3c3cc(-c4nc(-c5ccccc5)c5oc6c7ccccc7ccc6c5n4)ccc23)cc1 ; c1ccc(-c2cc3oc4ccccc4c3c3cc(-c4nc(-c5ccccc5)c5oc6ccc7ccccc7c6c5n4)ccc23)cc1 ; c1ccc(-c2cc3oc4ccccc4c3c3cc(-c4nc(-c5ccccc5)c5oc6cc7ccccc7cc6c5n4)ccc23)cc1]

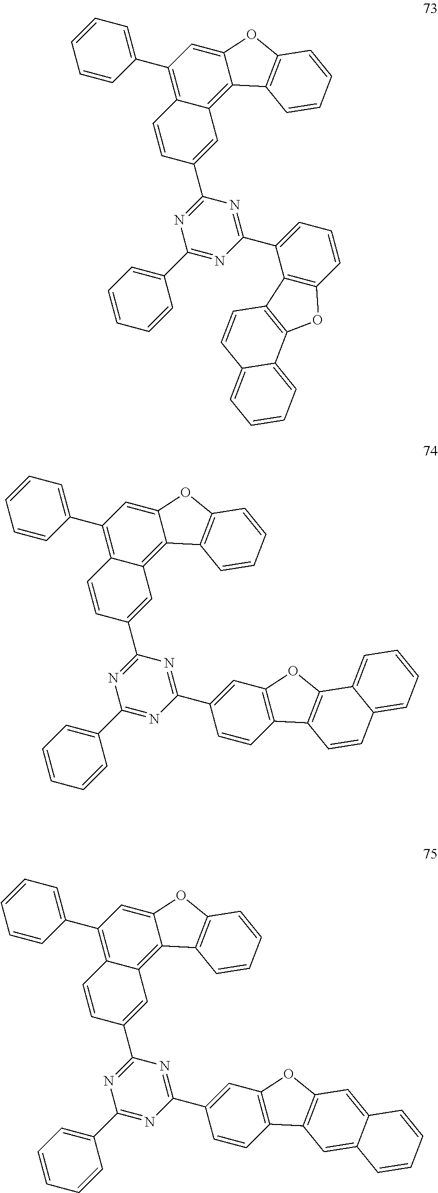 [SMILES: c1ccc(-c2nc(-c3ccc4c(-c5ccccc5)cc5oc6ccccc6c5c4c3)nc(-c3cccc4oc5c6ccccc6ccc5c34)n2)cc1 ; c1ccc(-c2nc(-c3ccc4c(c3)oc3c5ccccc5ccc43)nc(-c3ccc4c(-c5ccccc5)cc5oc6ccccc6c5c4c3)n2)cc1 ; c1ccc(-c2nc(-c3ccc4c(c3)oc3cc5ccccc5cc34)nc(-c3ccc4c(-c5ccccc5)cc5oc6ccccc6c5c4c3)n2)cc1]

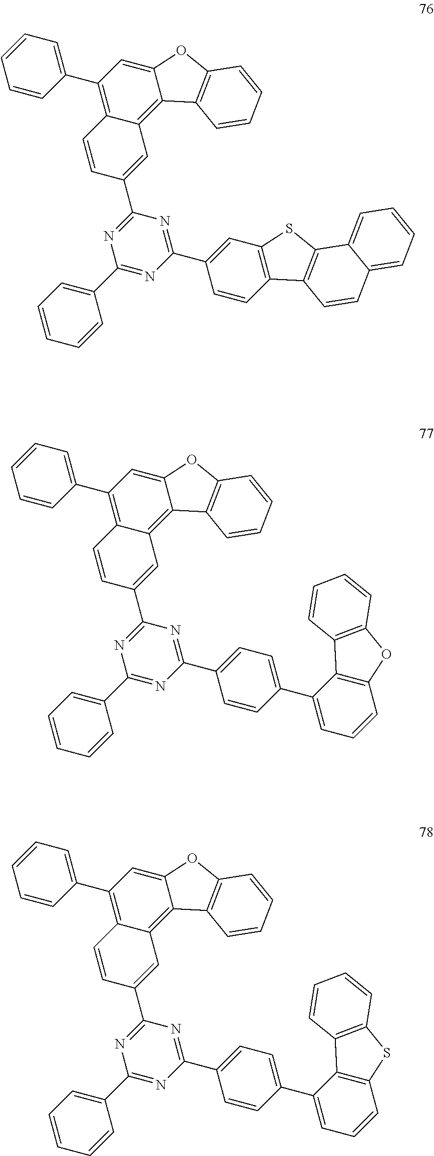 [SMILES: c1ccc(-c2nc(-c3ccc4c(c3)sc3c5ccccc5ccc43)nc(-c3ccc4c(-c5ccccc5)cc5oc6ccccc6c5c4c3)n2)cc1 ; c1ccc(-c2nc(-c3ccc(-c4cccc5oc6ccccc6c45)cc3)nc(-c3ccc4c(-c5ccccc5)cc5oc6ccccc6c5c4c3)n2)cc1 ; c1ccc(-c2nc(-c3ccc(-c4cccc5sc6ccccc6c45)cc3)nc(-c3ccc4c(-c5ccccc5)cc5oc6ccccc6c5c4c3)n2)cc1]

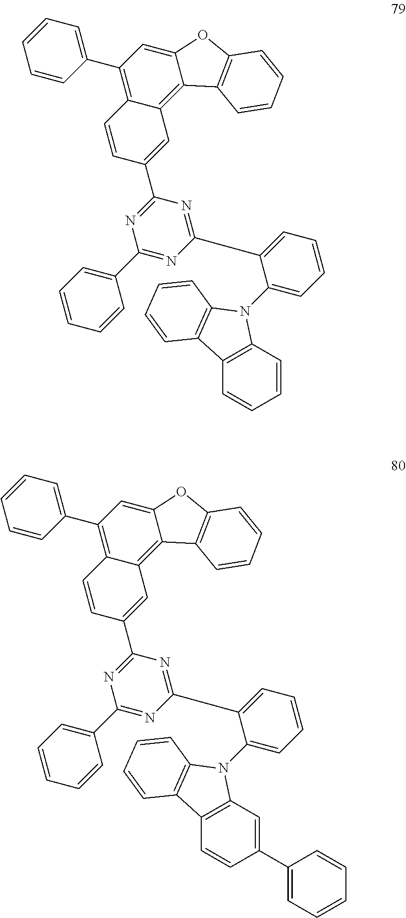 [SMILES: c1ccc(-c2nc(-c3ccc4c(-c5ccccc5)cc5oc6ccccc6c5c4c3)nc(-c3ccccc3-n3c4ccccc4c4ccccc43)n2)cc1 ; c1ccc(-c2ccc3c4ccccc4n(-c4ccccc4-c4nc(-c5ccccc5)nc(-c5ccc6c(-c7ccccc7)cc7oc8ccccc8c7c6c5)n4)c3c2)cc1]

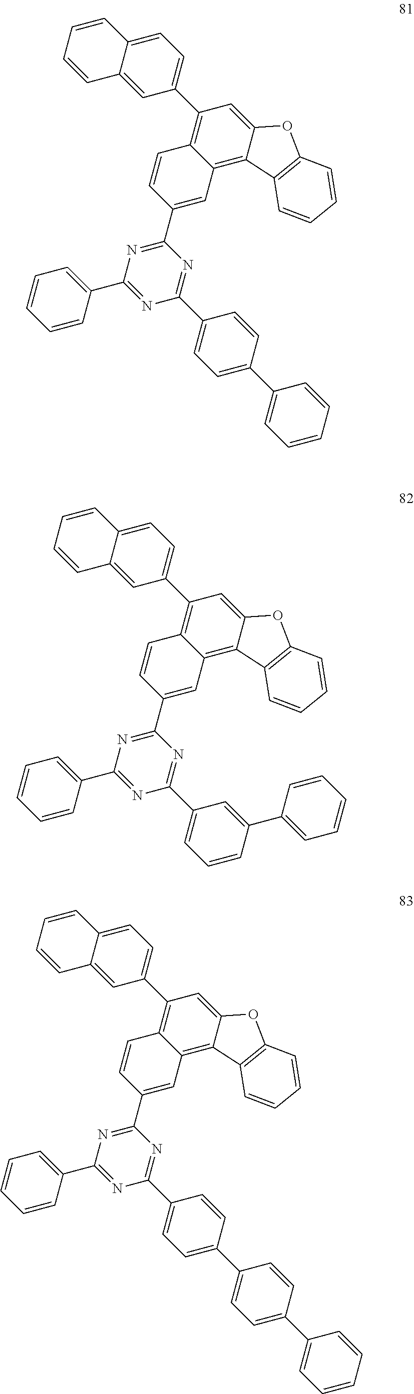 [SMILES: c1ccc(-c2ccc(-c3nc(-c4ccccc4)nc(-c4ccc5c(-c6ccc7ccccc7c6)cc6oc7ccccc7c6c5c4)n3)cc2)cc1 ; c1ccc(-c2cccc(-c3nc(-c4ccccc4)nc(-c4ccc5c(-c6ccc7ccccc7c6)cc6oc7ccccc7c6c5c4)n3)c2)cc1 ; c1ccc(-c2ccc(-c3ccc(-c4nc(-c5ccccc5)nc(-c5ccc6c(-c7ccc8ccccc8c7)cc7oc8ccccc8c7c6c5)n4)cc3)cc2)cc1]

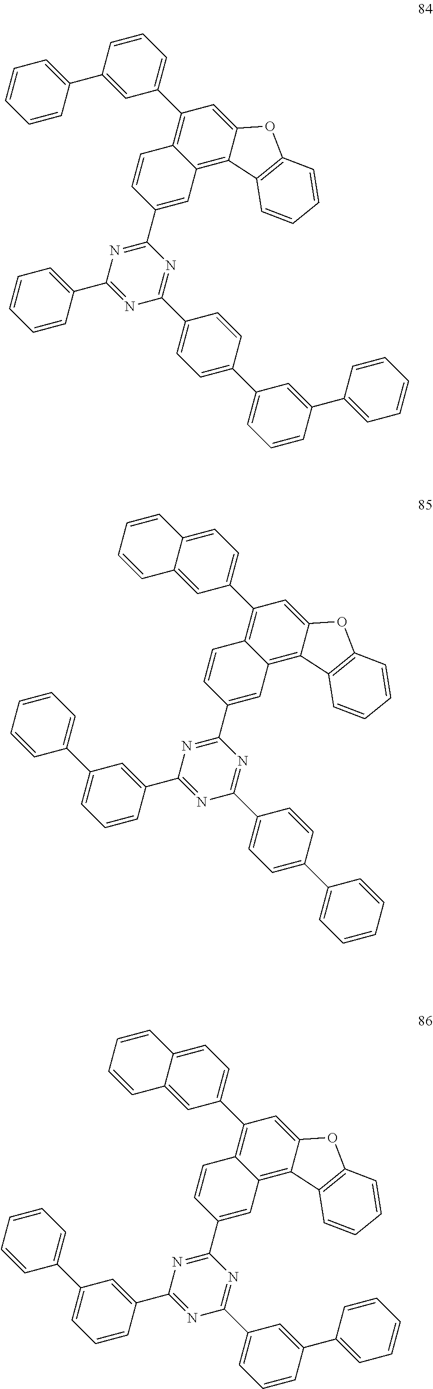 [SMILES: c1ccc(-c2cccc(-c3ccc(-c4nc(-c5ccccc5)nc(-c5ccc6c(-c7cccc(-c8ccccc8)c7)cc7oc8ccccc8c7c6c5)n4)cc3)c2)cc1 ; c1ccc(-c2ccc(-c3nc(-c4cccc(-c5ccccc5)c4)nc(-c4ccc5c(-c6ccc7ccccc7c6)cc6oc7ccccc7c6c5c4)n3)cc2)cc1 ; c1ccc(-c2cccc(-c3nc(-c4cccc(-c5ccccc5)c4)nc(-c4ccc5c(-c6ccc7ccccc7c6)cc6oc7ccccc7c6c5c4)n3)c2)cc1]

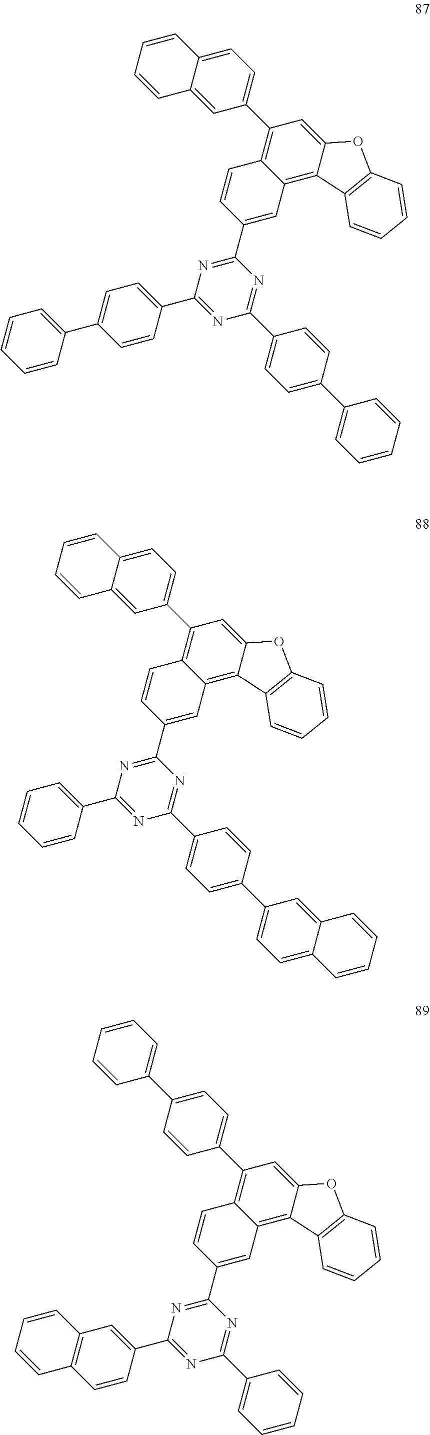 [SMILES: c1ccc(-c2ccc(-c3nc(-c4ccc(-c5ccccc5)cc4)nc(-c4ccc5c(-c6ccc7ccccc7c6)cc6oc7ccccc7c6c5c4)n3)cc2)cc1 ; c1ccc(-c2nc(-c3ccc(-c4ccc5ccccc5c4)cc3)nc(-c3ccc4c(-c5ccc6ccccc6c5)cc5oc6ccccc6c5c4c3)n2)cc1 ; c1ccc(-c2ccc(-c3cc4oc5ccccc5c4c4cc(-c5nc(-c6ccccc6)nc(-c6ccc7ccccc7c6)n5)ccc34)cc2)cc1]

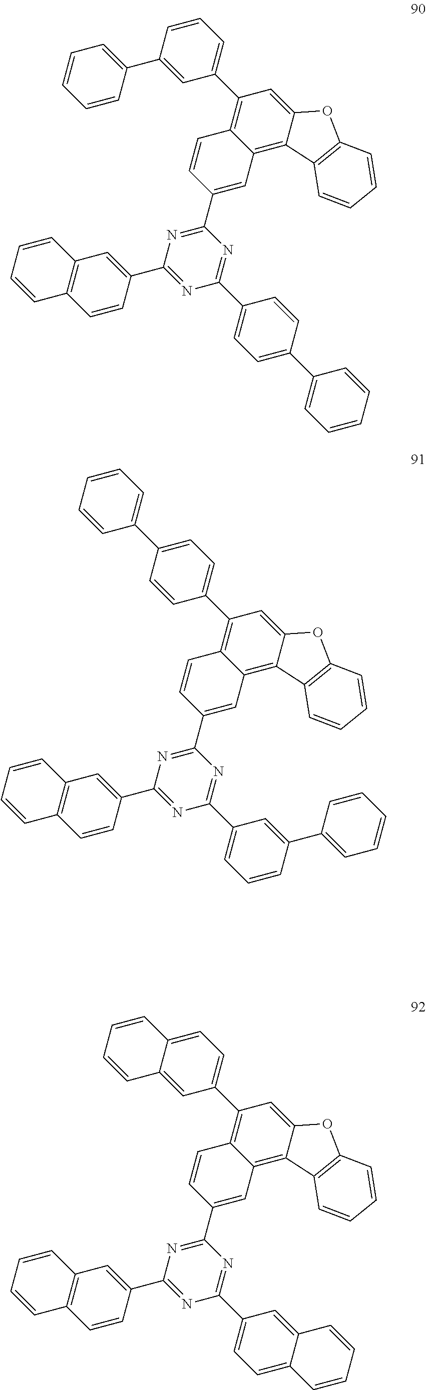 [SMILES: c1ccc(-c2ccc(-c3nc(-c4ccc5ccccc5c4)nc(-c4ccc5c(-c6cccc(-c7ccccc7)c6)cc6oc7ccccc7c6c5c4)n3)cc2)cc1 ; c1ccc(-c2ccc(-c3cc4oc5ccccc5c4c4cc(-c5nc(-c6cccc(-c7ccccc7)c6)nc(-c6ccc7ccccc7c6)n5)ccc34)cc2)cc1 ; c1ccc2cc(-c3nc(-c4ccc5ccccc5c4)nc(-c4ccc5c(-c6ccc7ccccc7c6)cc6oc7ccccc7c6c5c4)n3)ccc2c1]

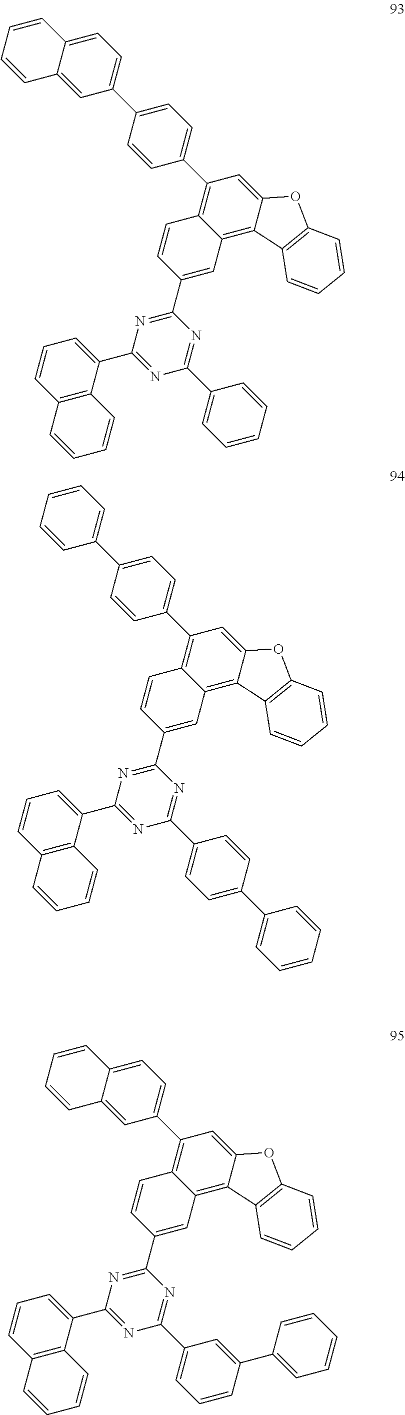

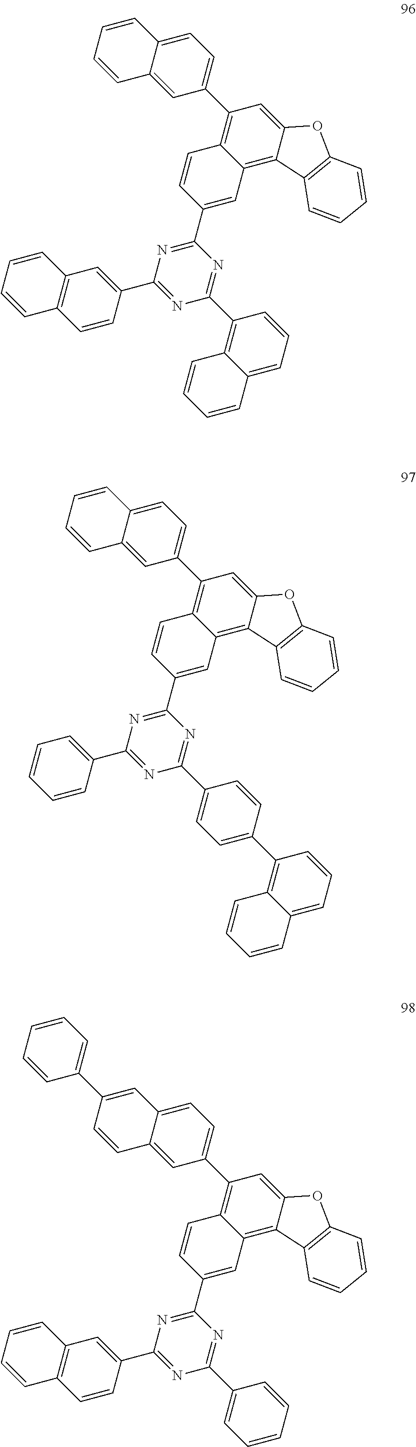 [SMILES: c1ccc2cc(-c3nc(-c4ccc5c(-c6ccc7ccccc7c6)cc6oc7ccccc7c6c5c4)nc(-c4cccc5ccccc45)n3)ccc2c1 ; c1ccc(-c2nc(-c3ccc(-c4cccc5ccccc45)cc3)nc(-c3ccc4c(-c5ccc6ccccc6c5)cc5oc6ccccc6c5c4c3)n2)cc1 ; c1ccc(-c2ccc3cc(-c4cc5oc6ccccc6c5c5cc(-c6nc(-c7ccccc7)nc(-c7ccc8ccccc8c7)n6)ccc45)ccc3c2)cc1]

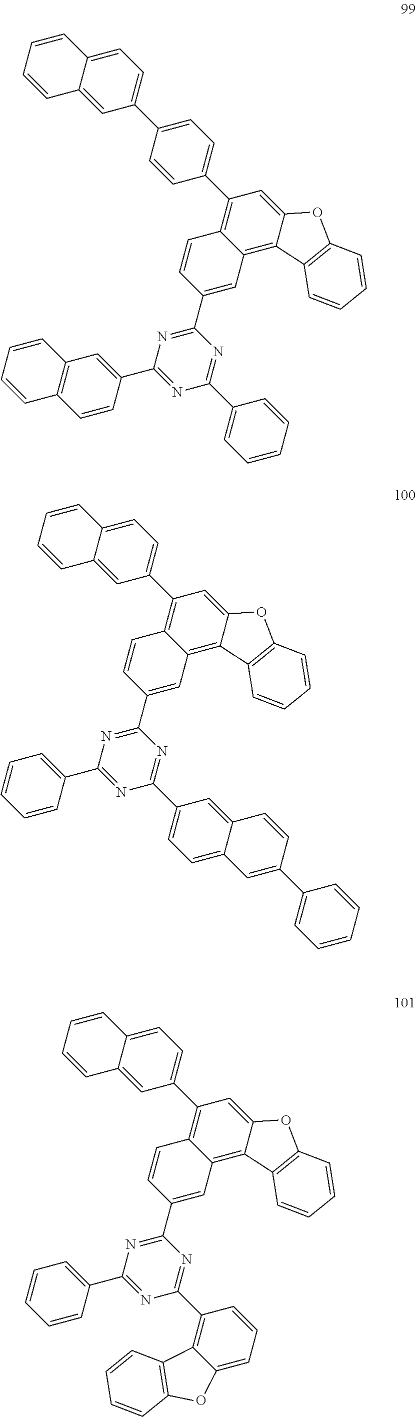 [SMILES: c1ccc(-c2nc(-c3ccc4ccccc4c3)nc(-c3ccc4c(-c5ccc(-c6ccc7ccccc7c6)cc5)cc5oc6ccccc6c5c4c3)n2)cc1 ; c1ccc(-c2ccc3cc(-c4nc(-c5ccccc5)nc(-c5ccc6c(-c7ccc8ccccc8c7)cc7oc8ccccc8c7c6c5)n4)ccc3c2)cc1 ; c1ccc(-c2nc(-c3ccc4c(-c5ccc6ccccc6c5)cc5oc6ccccc6c5c4c3)nc(-c3cccc4oc5ccccc5c34)n2)cc1]

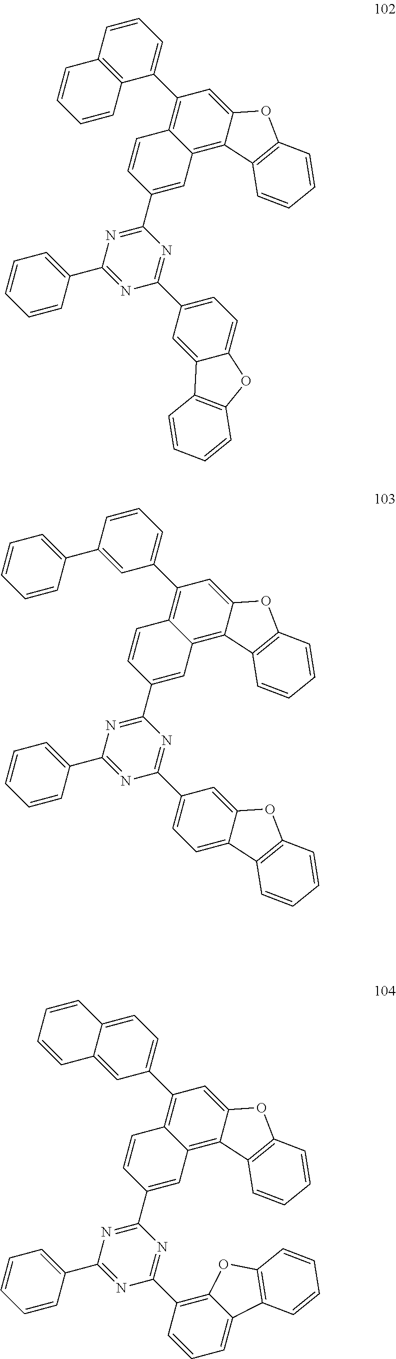 [SMILES: c1ccc(-c2nc(-c3ccc4oc5ccccc5c4c3)nc(-c3ccc4c(-c5cccc6ccccc56)cc5oc6ccccc6c5c4c3)n2)cc1 ; c1ccc(-c2cccc(-c3cc4oc5ccccc5c4c4cc(-c5nc(-c6ccccc6)nc(-c6ccc7c(c6)oc6ccccc67)n5)ccc34)c2)cc1 ; c1ccc(-c2nc(-c3ccc4c(-c5ccc6ccccc6c5)cc5oc6ccccc6c5c4c3)nc(-c3cccc4c3oc3ccccc34)n2)cc1]

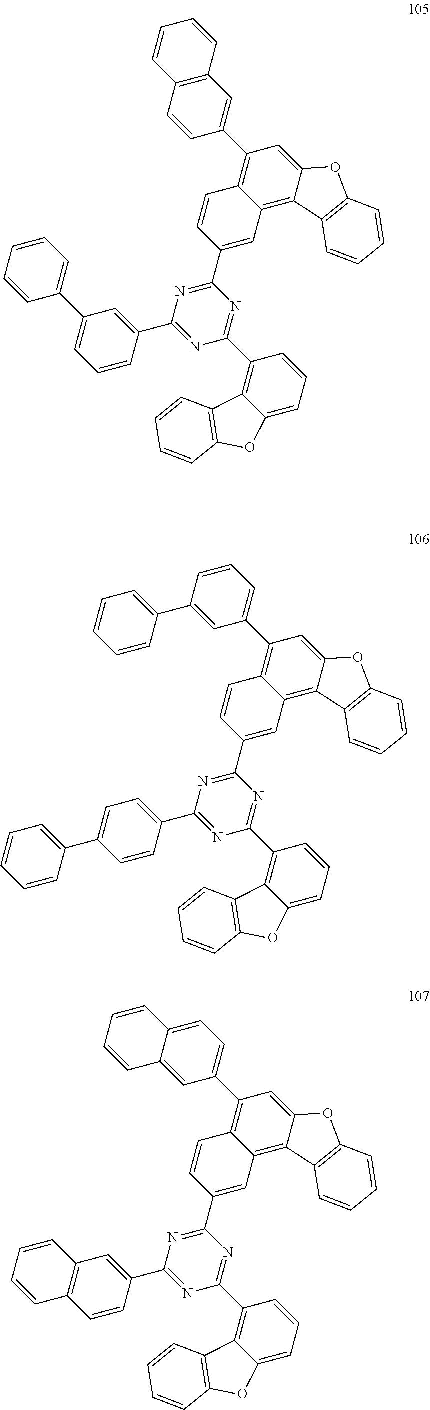 [SMILES: c1ccc(-c2cccc(-c3nc(-c4ccc5c(-c6ccc7ccccc7c6)cc6oc7ccccc7c6c5c4)nc(-c4cccc5oc6ccccc6c45)n3)c2)cc1 ; c1ccc(-c2ccc(-c3nc(-c4ccc5c(-c6cccc(-c7ccccc7)c6)cc6oc7ccccc7c6c5c4)nc(-c4cccc5oc6ccccc6c45)n3)cc2)cc1 ; c1ccc2cc(-c3nc(-c4ccc5c(-c6ccc7ccccc7c6)cc6oc7ccccc7c6c5c4)nc(-c4cccc5oc6ccccc6c45)n3)ccc2c1]

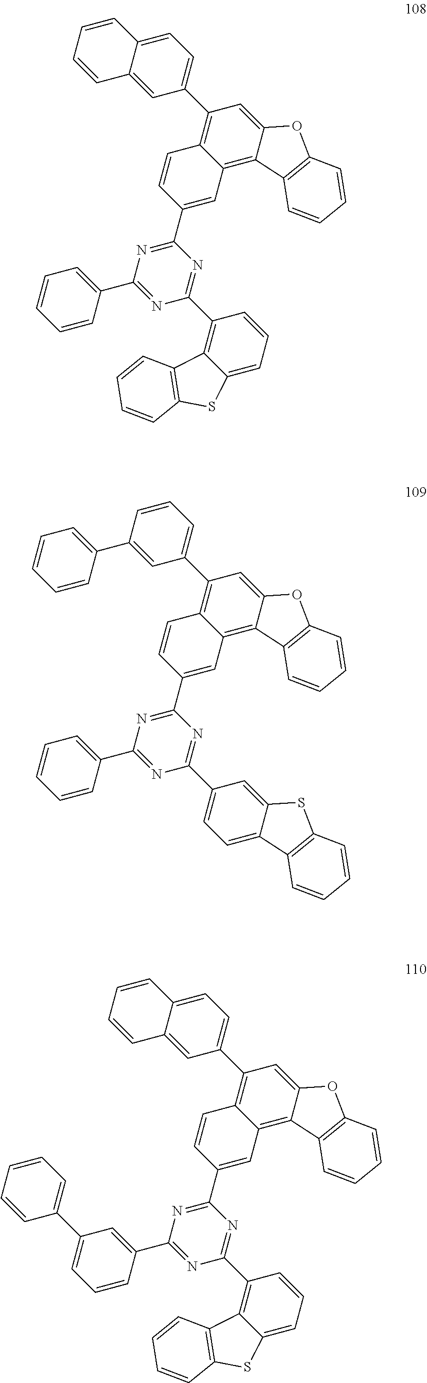 [SMILES: c1ccc(-c2nc(-c3ccc4c(-c5ccc6ccccc6c5)cc5oc6ccccc6c5c4c3)nc(-c3cccc4sc5ccccc5c34)n2)cc1 ; c1ccc(-c2cccc(-c3cc4oc5ccccc5c4c4cc(-c5nc(-c6ccccc6)nc(-c6ccc7c(c6)sc6ccccc67)n5)ccc34)c2)cc1 ; c1ccc(-c2cccc(-c3nc(-c4ccc5c(-c6ccc7ccccc7c6)cc6oc7ccccc7c6c5c4)nc(-c4cccc5sc6ccccc6c45)n3)c2)cc1]

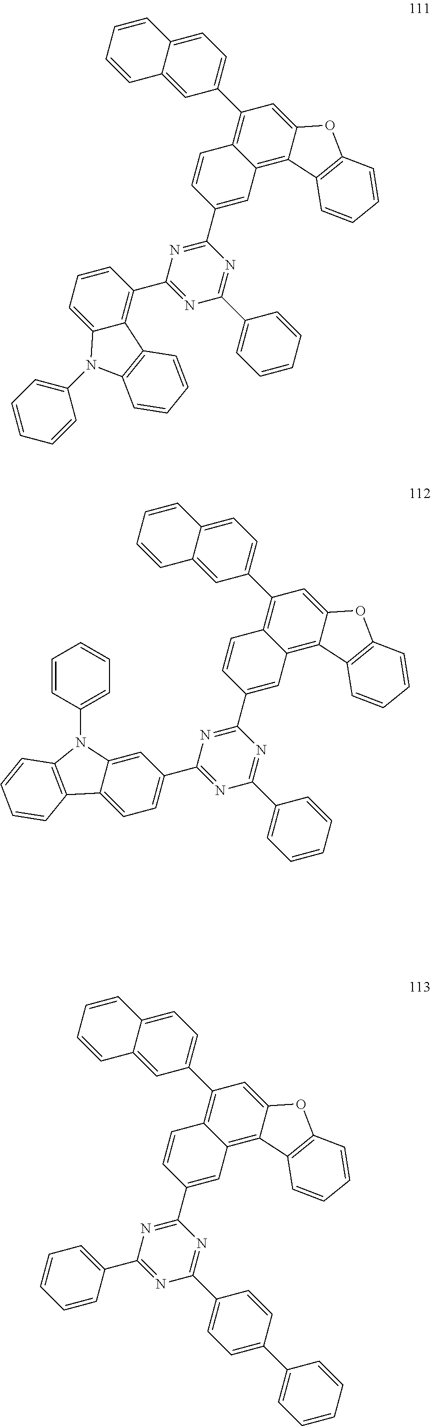 [SMILES: c1ccc(-c2nc(-c3ccc4c(-c5ccc6ccccc6c5)cc5oc6ccccc6c5c4c3)nc(-c3cccc4c3c3ccccc3n4-c3ccccc3)n2)cc1 ; c1ccc(-c2nc(-c3ccc4c(-c5ccc6ccccc6c5)cc5oc6ccccc6c5c4c3)nc(-c3ccc4c5ccccc5n(-c5ccccc5)c4c3)n2)cc1 ; c1ccc(-c2ccc(-c3nc(-c4ccccc4)nc(-c4ccc5c(-c6ccc7ccccc7c6)cc6oc7ccccc7c6c5c4)n3)cc2)cc1]

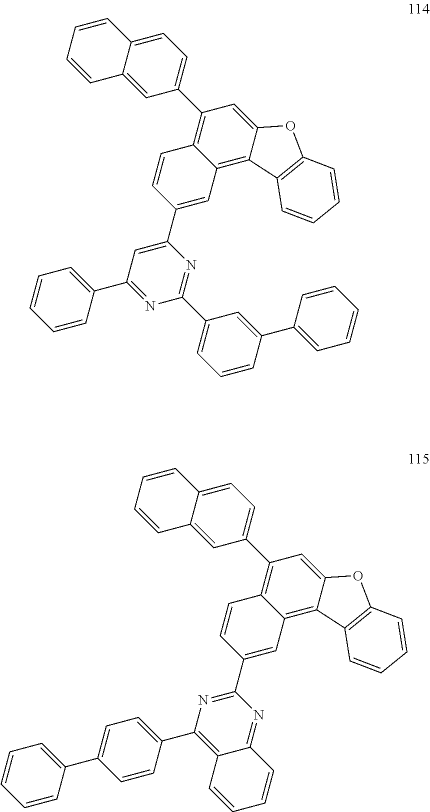 [SMILES: c1ccc(-c2cccc(-c3nc(-c4ccccc4)cc(-c4ccc5c(-c6ccc7ccccc7c6)cc6oc7ccccc7c6c5c4)n3)c2)cc1 ; c1ccc(-c2ccc(-c3nc(-c4ccc5c(-c6ccc7ccccc7c6)cc6oc7ccccc7c6c5c4)nc4ccccc34)cc2)cc1]

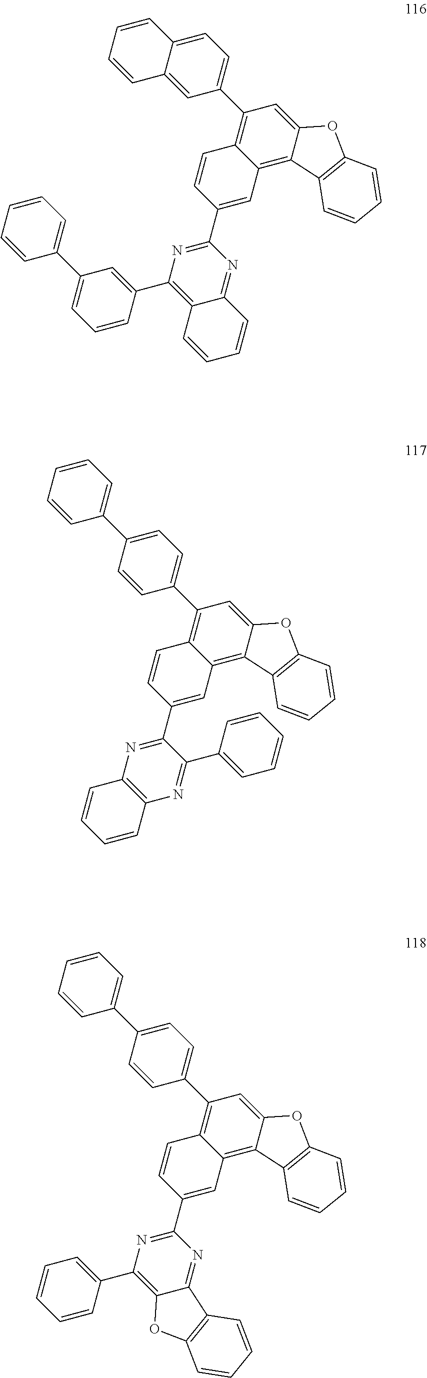 [SMILES: c1ccc(-c2cccc(-c3nc(-c4ccc5c(-c6ccc7ccccc7c6)cc6oc7ccccc7c6c5c4)nc4ccccc34)c2)cc1 ; c1ccc(-c2ccc(-c3cc4oc5ccccc5c4c4cc(-c5nc6ccccc6nc5-c5ccccc5)ccc34)cc2)cc1 ; c1ccc(-c2ccc(-c3cc4oc5ccccc5c4c4cc(-c5nc(-c6ccccc6)c6oc7ccccc7c6n5)ccc34)cc2)cc1]

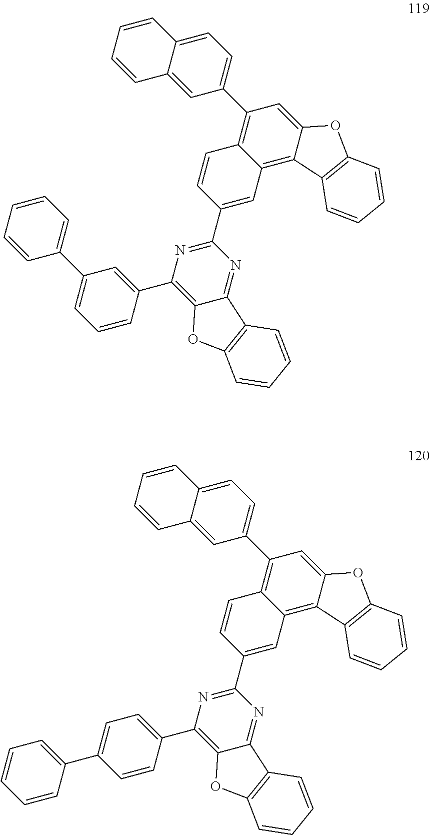 [SMILES: c1ccc(-c2cccc(-c3nc(-c4ccc5c(-c6ccc7ccccc7c6)cc6oc7ccccc7c6c5c4)nc4c3oc3ccccc34)c2)cc1 ; c1ccc(-c2ccc(-c3nc(-c4ccc5c(-c6ccc7ccccc7c6)cc6oc7ccccc7c6c5c4)nc4c3oc3ccccc34)cc2)cc1]

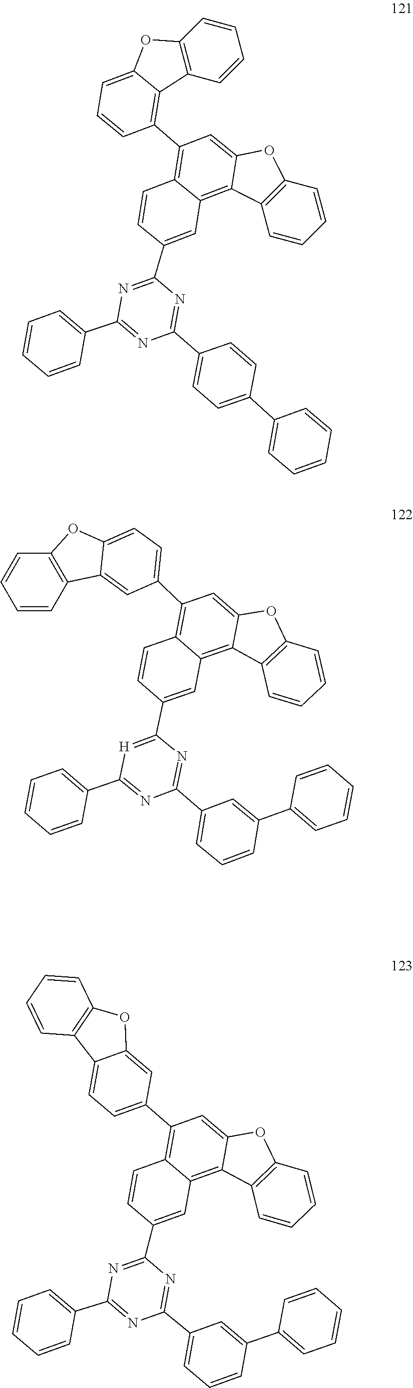

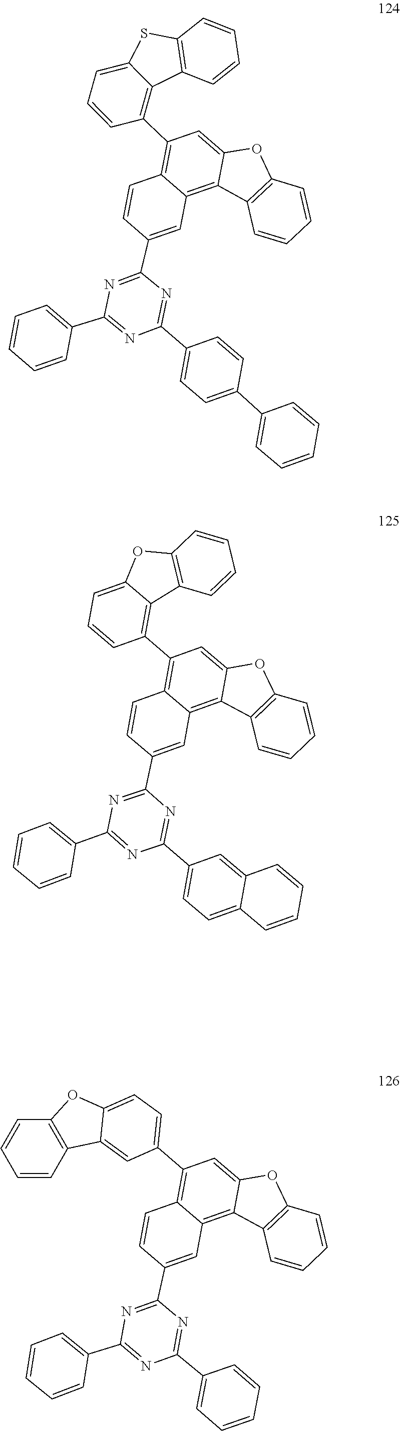 [SMILES: c1ccc(-c2ccc(-c3nc(-c4ccccc4)nc(-c4ccc5c(-c6cccc7sc8ccccc8c67)cc6oc7ccccc7c6c5c4)n3)cc2)cc1 ; c1ccc(-c2nc(-c3ccc4ccccc4c3)nc(-c3ccc4c(-c5cccc6oc7ccccc7c56)cc5oc6ccccc6c5c4c3)n2)cc1 ; c1ccc(-c2nc(-c3ccccc3)nc(-c3ccc4c(-c5ccc6oc7ccccc7c6c5)cc5oc6ccccc6c5c4c3)n2)cc1]

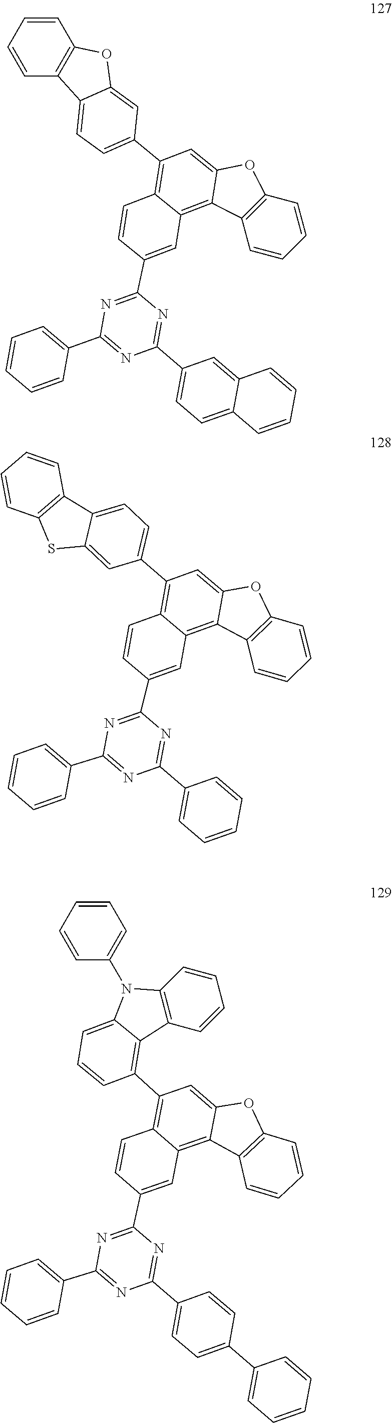 [SMILES: c1ccc(-c2nc(-c3ccc4ccccc4c3)nc(-c3ccc4c(-c5ccc6c(c5)oc5ccccc56)cc5oc6ccccc6c5c4c3)n2)cc1 ; c1ccc(-c2nc(-c3ccccc3)nc(-c3ccc4c(-c5ccc6c(c5)sc5ccccc56)cc5oc6ccccc6c5c4c3)n2)cc1 ; c1ccc(-c2ccc(-c3nc(-c4ccccc4)nc(-c4ccc5c(-c6cccc7c6c6ccccc6n7-c6ccccc6)cc6oc7ccccc7c6c5c4)n3)cc2)cc1]

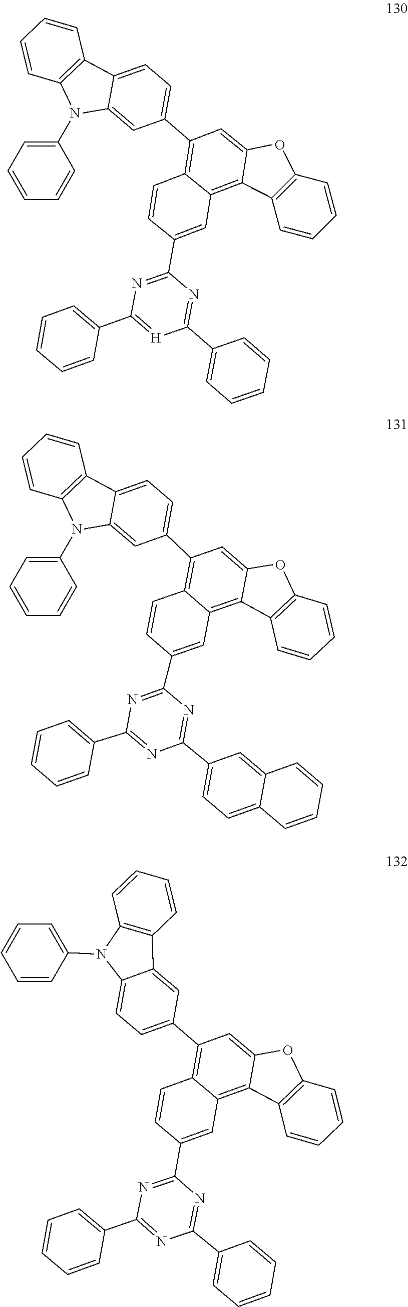

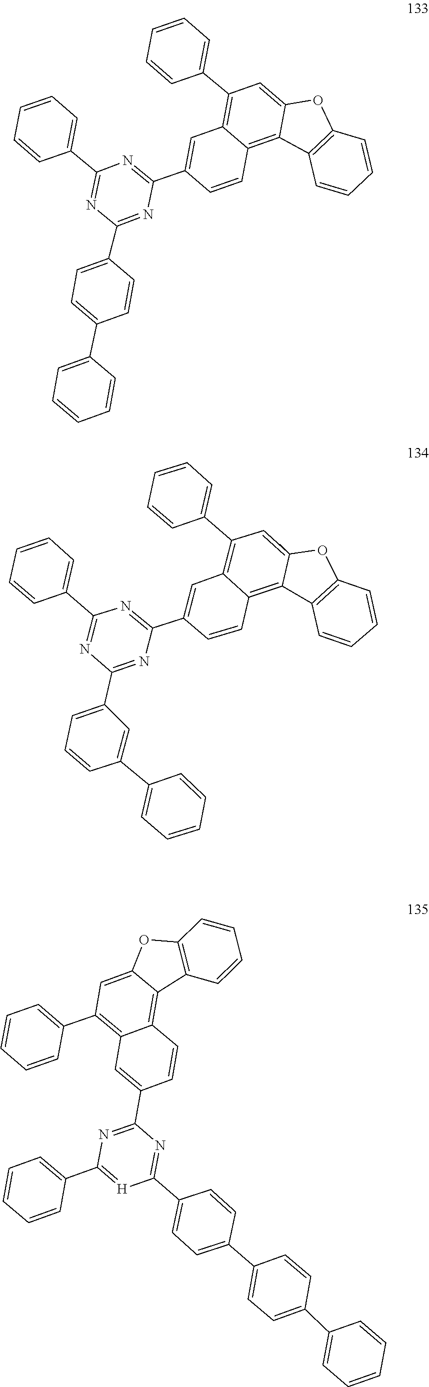

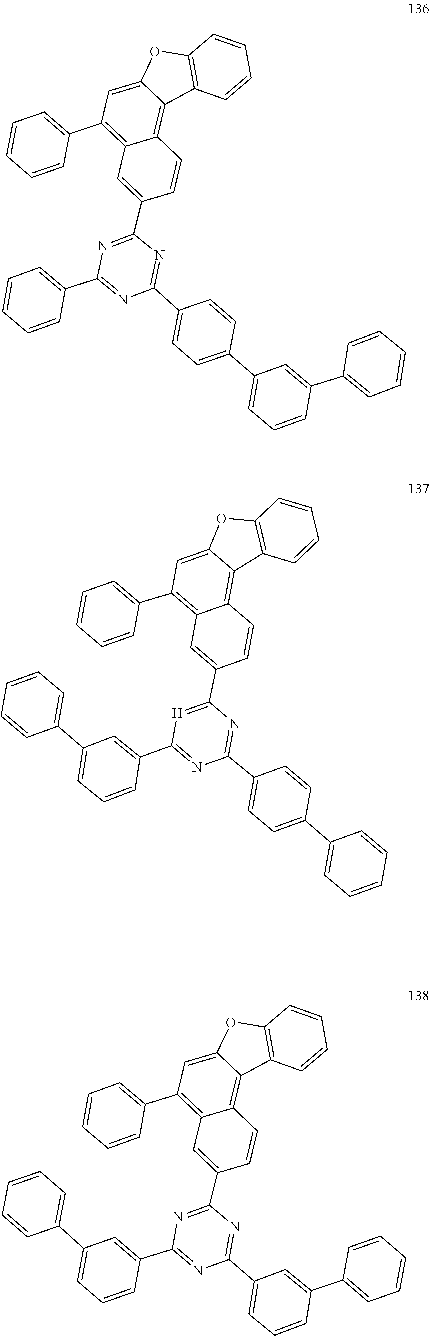

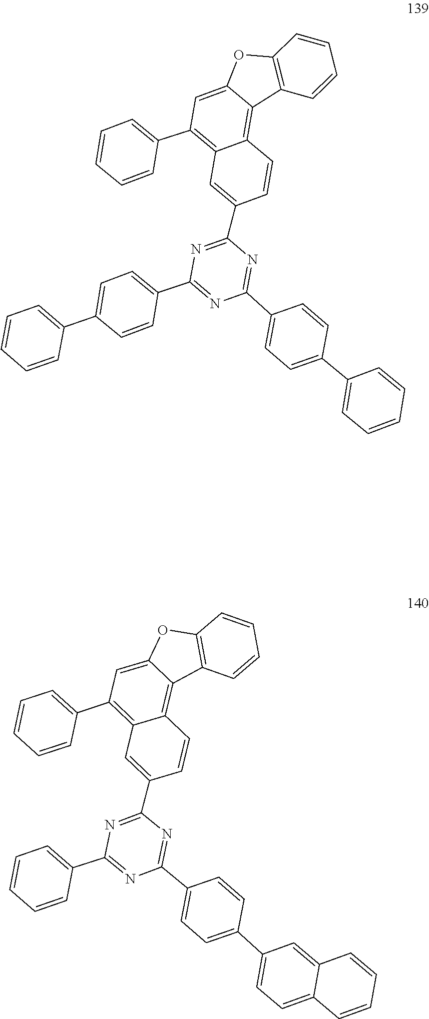 [SMILES: c1ccc(-c2ccc(-c3nc(-c4ccc(-c5ccccc5)cc4)nc(-c4ccc5c(c4)c(-c4ccccc4)cc4oc6ccccc6c45)n3)cc2)cc1 ; c1ccc(-c2nc(-c3ccc(-c4ccc5ccccc5c4)cc3)nc(-c3ccc4c(c3)c(-c3ccccc3)cc3oc5ccccc5c34)n2)cc1]

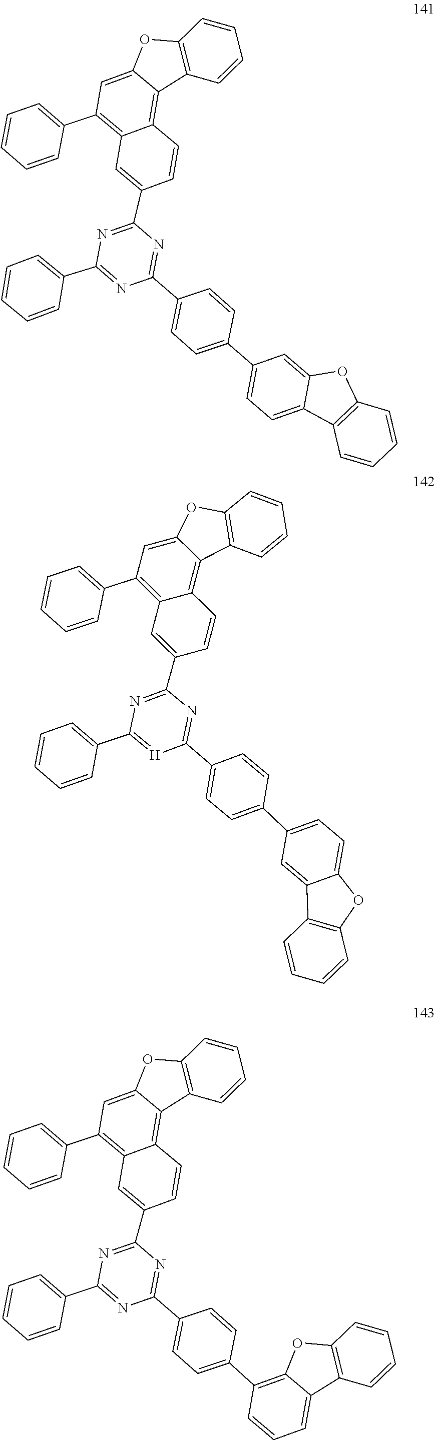

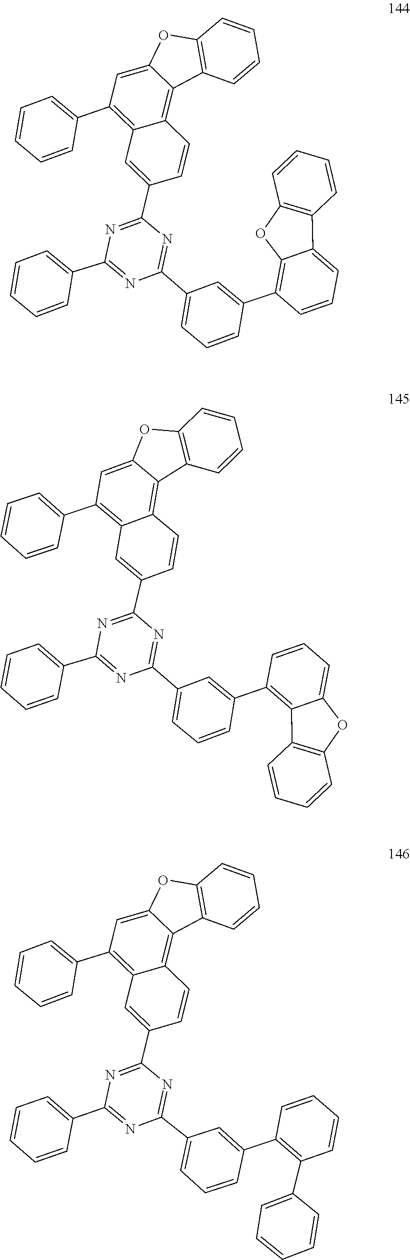 [SMILES: c1ccc(-c2nc(-c3cccc(-c4cccc5c4oc4ccccc45)c3)nc(-c3ccc4c(c3)c(-c3ccccc3)cc3oc5ccccc5c34)n2)cc1 ; c1ccc(-c2nc(-c3cccc(-c4cccc5oc6ccccc6c45)c3)nc(-c3ccc4c(c3)c(-c3ccccc3)cc3oc5ccccc5c34)n2)cc1 ; c1ccc(-c2nc(-c3cccc(-c4ccccc4-c4ccccc4)c3)nc(-c3ccc4c(c3)c(-c3ccccc3)cc3oc5ccccc5c34)n2)cc1]

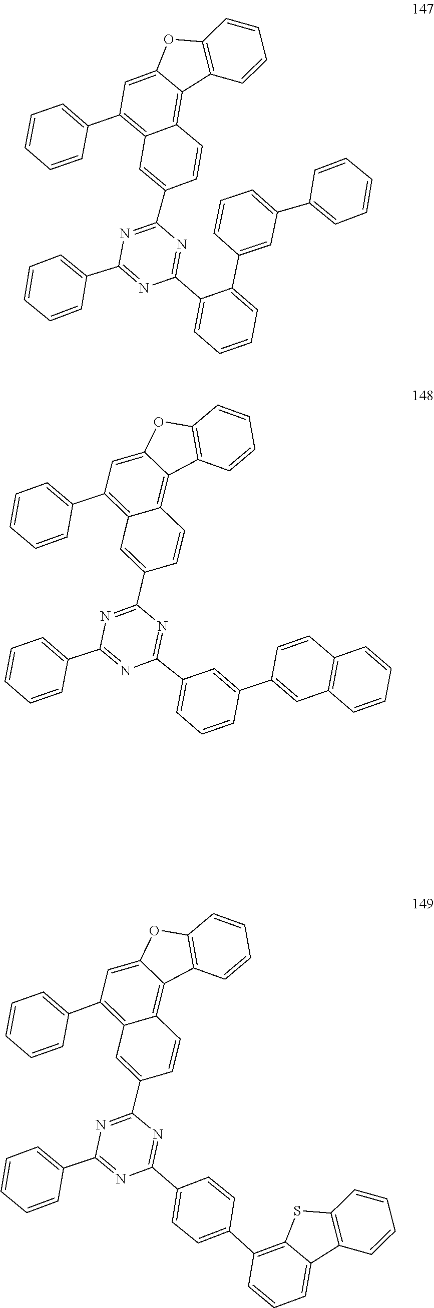 [SMILES: c1ccc(-c2cccc(-c3ccccc3-c3nc(-c4ccccc4)nc(-c4ccc5c(c4)c(-c4ccccc4)cc4oc6ccccc6c45)n3)c2)cc1 ; c1ccc(-c2nc(-c3cccc(-c4ccc5ccccc5c4)c3)nc(-c3ccc4c(c3)c(-c3ccccc3)cc3oc5ccccc5c34)n2)cc1 ; c1ccc(-c2nc(-c3ccc(-c4cccc5c4sc4ccccc45)cc3)nc(-c3ccc4c(c3)c(-c3ccccc3)cc3oc5ccccc5c34)n2)cc1]

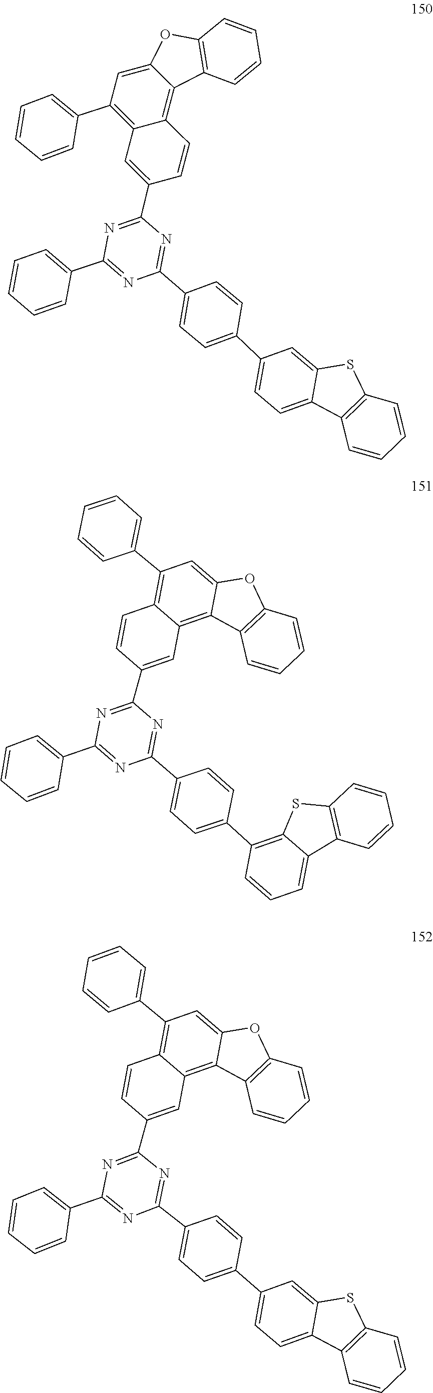 [SMILES: c1ccc(-c2nc(-c3ccc(-c4ccc5c(c4)sc4ccccc45)cc3)nc(-c3ccc4c(c3)c(-c3ccccc3)cc3oc5ccccc5c34)n2)cc1 ; c1ccc(-c2nc(-c3ccc(-c4cccc5c4sc4ccccc45)cc3)nc(-c3ccc4c(-c5ccccc5)cc5oc6ccccc6c5c4c3)n2)cc1 ; c1ccc(-c2nc(-c3ccc(-c4ccc5c(c4)sc4ccccc45)cc3)nc(-c3ccc4c(-c5ccccc5)cc5oc6ccccc6c5c4c3)n2)cc1]

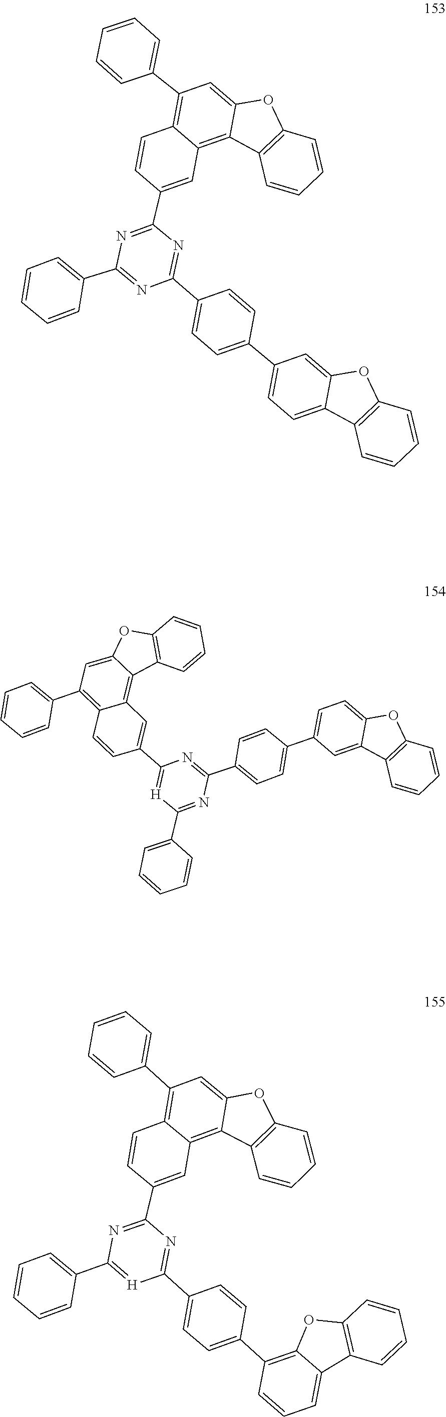

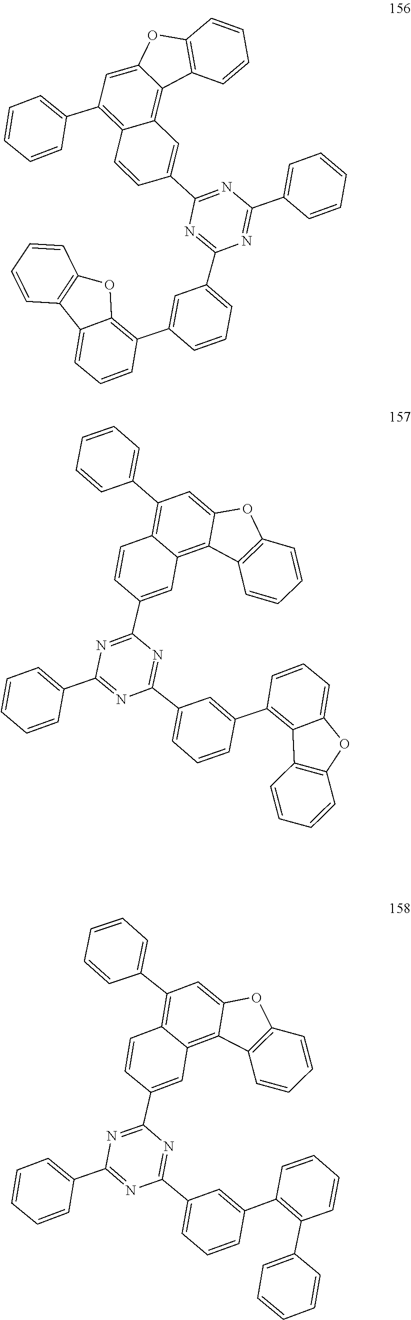

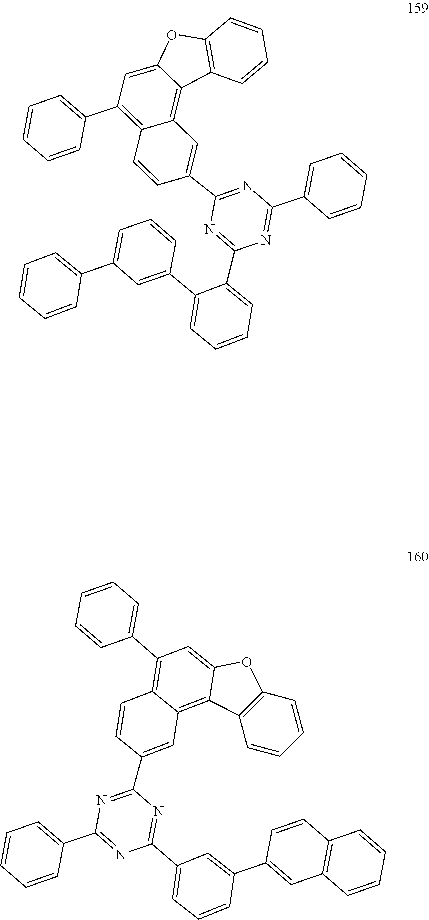 [SMILES: c1ccc(-c2cccc(-c3ccccc3-c3nc(-c4ccccc4)nc(-c4ccc5c(-c6ccccc6)cc6oc7ccccc7c6c5c4)n3)c2)cc1 ; c1ccc(-c2nc(-c3cccc(-c4ccc5ccccc5c4)c3)nc(-c3ccc4c(-c5ccccc5)cc5oc6ccccc6c5c4c3)n2)cc1]

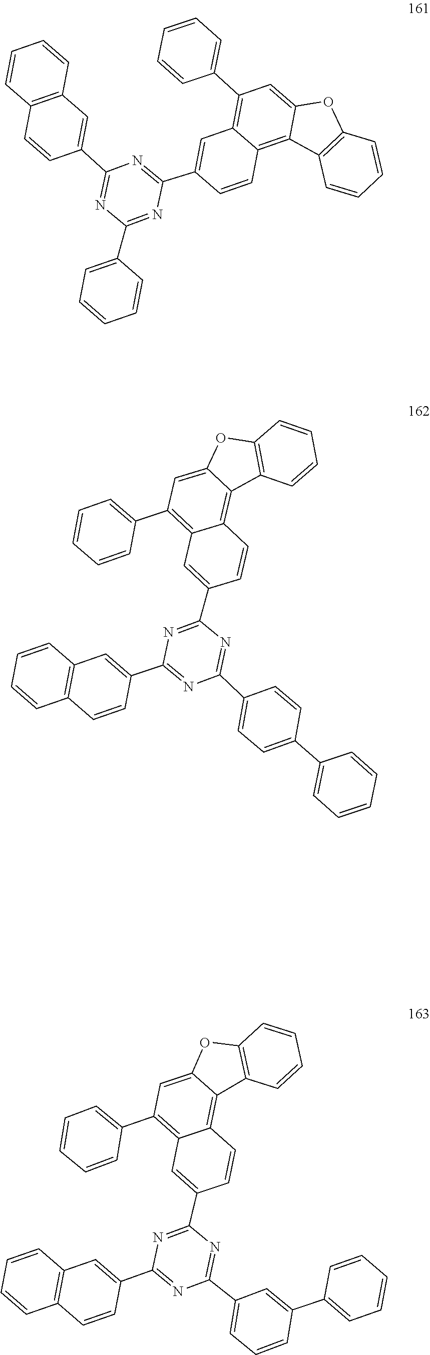 [SMILES: c1ccc(-c2nc(-c3ccc4ccccc4c3)nc(-c3ccc4c(c3)c(-c3ccccc3)cc3oc5ccccc5c34)n2)cc1 ; c1ccc(-c2ccc(-c3nc(-c4ccc5ccccc5c4)nc(-c4ccc5c(c4)c(-c4ccccc4)cc4oc6ccccc6c45)n3)cc2)cc1 ; c1ccc(-c2cccc(-c3nc(-c4ccc5ccccc5c4)nc(-c4ccc5c(c4)c(-c4ccccc4)cc4oc6ccccc6c45)n3)c2)cc1]

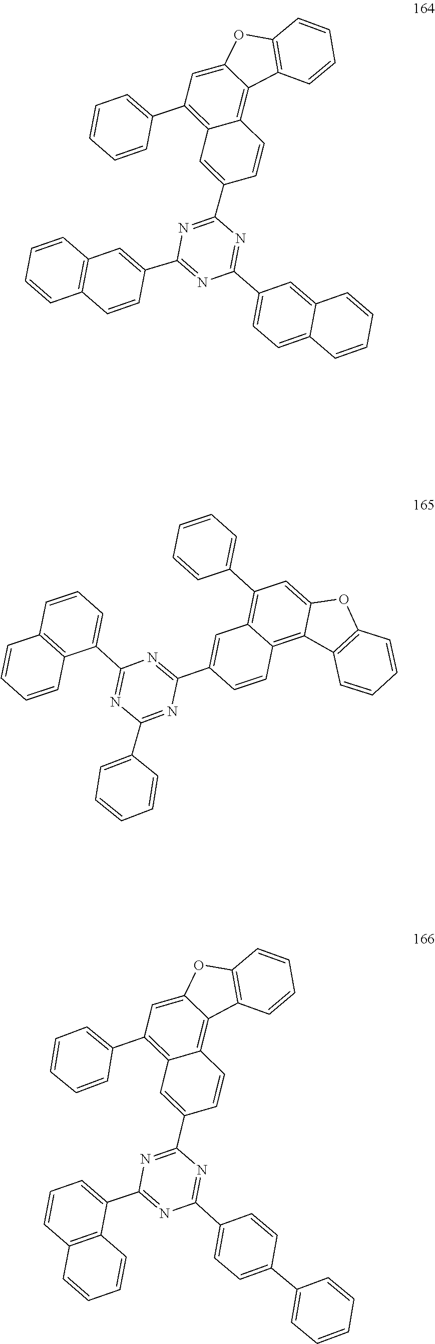 [SMILES: c1ccc(-c2cc3oc4ccccc4c3c3ccc(-c4nc(-c5ccc6ccccc6c5)nc(-c5ccc6ccccc6c5)n4)cc23)cc1 ; c1ccc(-c2nc(-c3ccc4c(c3)c(-c3ccccc3)cc3oc5ccccc5c34)nc(-c3cccc4ccccc34)n2)cc1 ; c1ccc(-c2ccc(-c3nc(-c4ccc5c(c4)c(-c4ccccc4)cc4oc6ccccc6c45)nc(-c4cccc5ccccc45)n3)cc2)cc1]

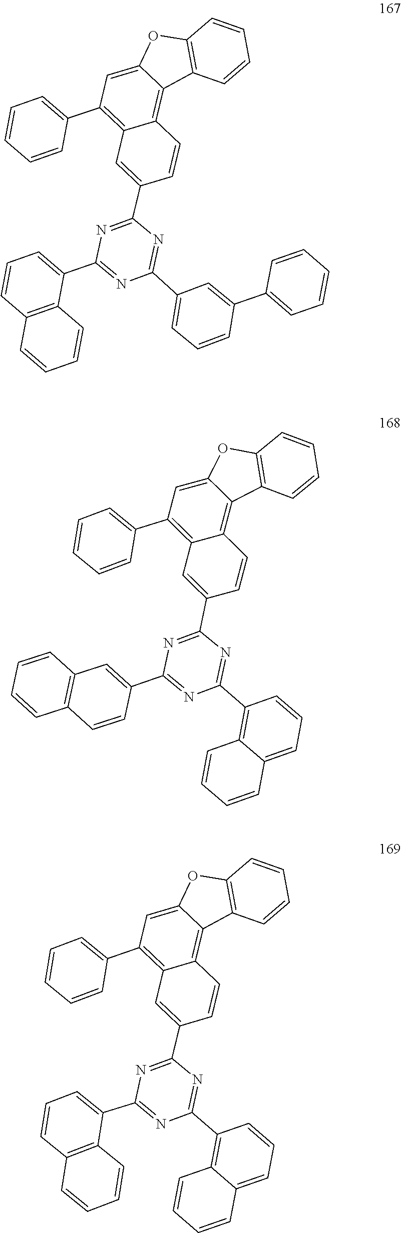 [SMILES: c1ccc(-c2cccc(-c3nc(-c4ccc5c(c4)c(-c4ccccc4)cc4oc6ccccc6c45)nc(-c4cccc5ccccc45)n3)c2)cc1 ; c1ccc(-c2cc3oc4ccccc4c3c3ccc(-c4nc(-c5ccc6ccccc6c5)nc(-c5cccc6ccccc56)n4)cc23)cc1 ; c1ccc(-c2cc3oc4ccccc4c3c3ccc(-c4nc(-c5cccc6ccccc56)nc(-c5cccc6ccccc56)n4)cc23)cc1]

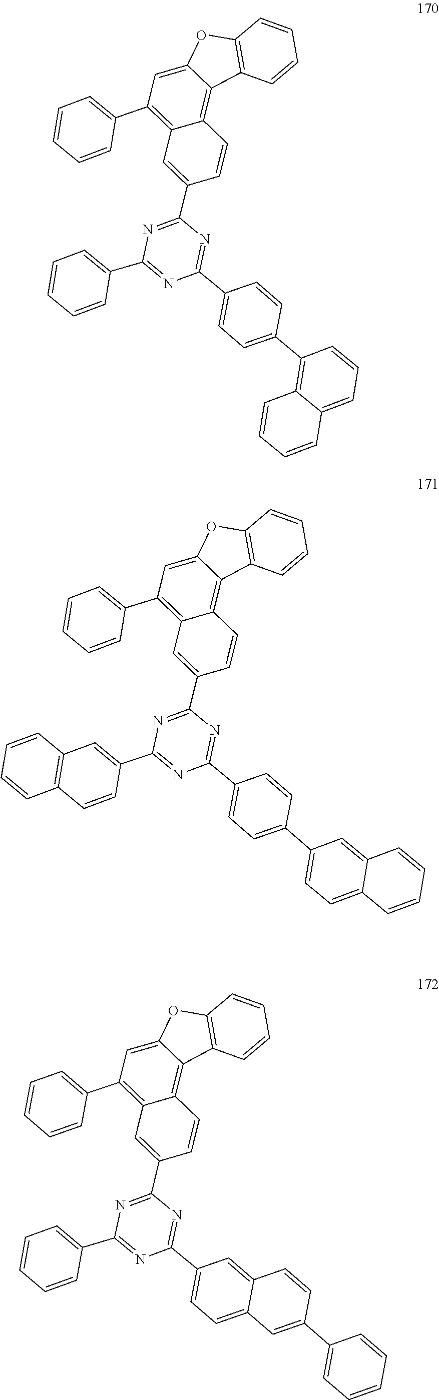 [SMILES: c1ccc(-c2nc(-c3ccc(-c4cccc5ccccc45)cc3)nc(-c3ccc4c(c3)c(-c3ccccc3)cc3oc5ccccc5c34)n2)cc1 ; c1ccc(-c2cc3oc4ccccc4c3c3ccc(-c4nc(-c5ccc(-c6ccc7ccccc7c6)cc5)nc(-c5ccc6ccccc6c5)n4)cc23)cc1 ; c1ccc(-c2ccc3cc(-c4nc(-c5ccccc5)nc(-c5ccc6c(c5)c(-c5ccccc5)cc5oc7ccccc7c56)n4)ccc3c2)cc1]

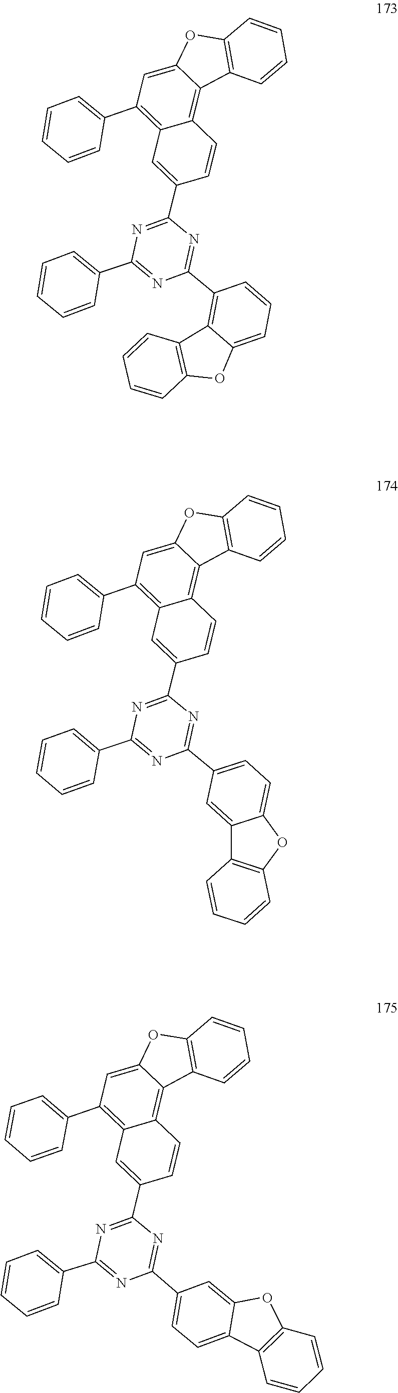 [SMILES: c1ccc(-c2nc(-c3ccc4c(c3)c(-c3ccccc3)cc3oc5ccccc5c34)nc(-c3cccc4oc5ccccc5c34)n2)cc1 ; c1ccc(-c2nc(-c3ccc4c(c3)c(-c3ccccc3)cc3oc5ccccc5c34)nc(-c3ccc4oc5ccccc5c4c3)n2)cc1 ; c1ccc(-c2nc(-c3ccc4c(c3)oc3ccccc34)nc(-c3ccc4c(c3)c(-c3ccccc3)cc3oc5ccccc5c34)n2)cc1]

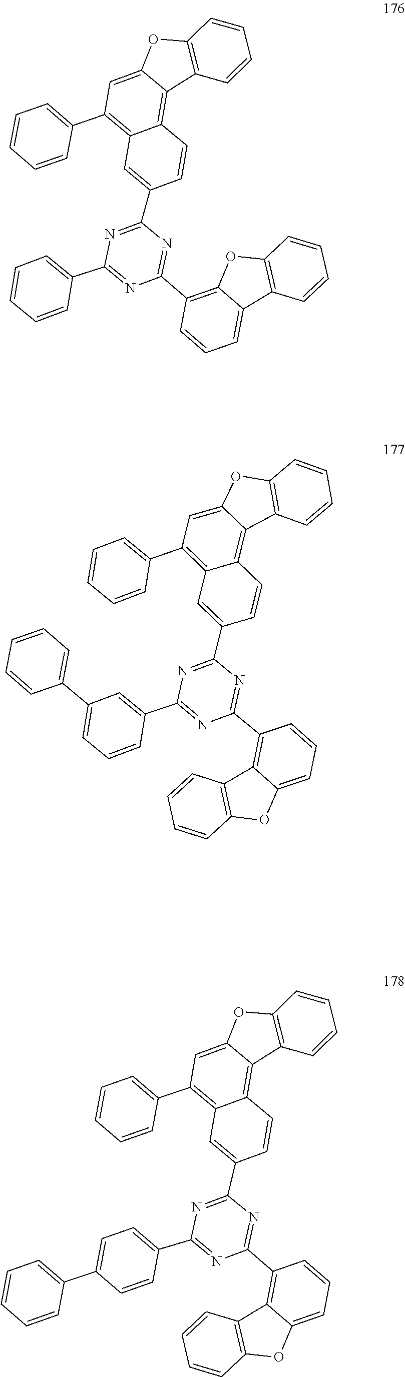 [SMILES: c1ccc(-c2nc(-c3ccc4c(c3)c(-c3ccccc3)cc3oc5ccccc5c34)nc(-c3cccc4c3oc3ccccc34)n2)cc1 ; c1ccc(-c2cccc(-c3nc(-c4ccc5c(c4)c(-c4ccccc4)cc4oc6ccccc6c45)nc(-c4cccc5oc6ccccc6c45)n3)c2)cc1 ; c1ccc(-c2ccc(-c3nc(-c4ccc5c(c4)c(-c4ccccc4)cc4oc6ccccc6c45)nc(-c4cccc5oc6ccccc6c45)n3)cc2)cc1]

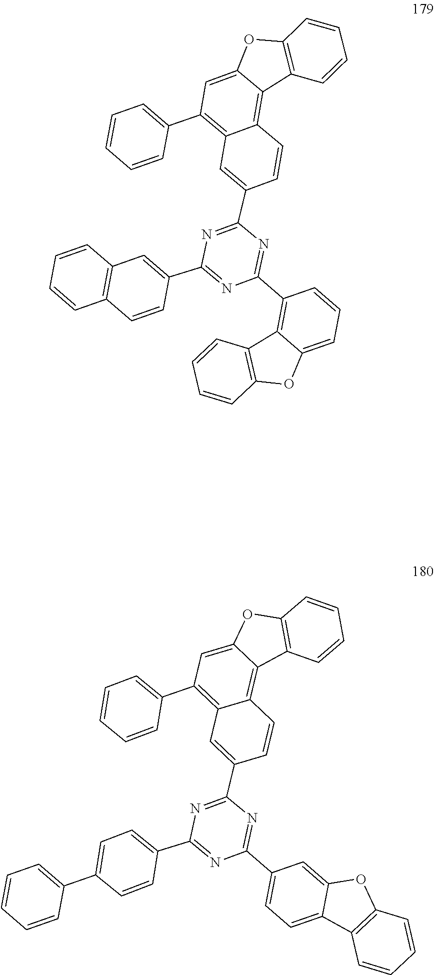 [SMILES: c1ccc(-c2cc3oc4ccccc4c3c3ccc(-c4nc(-c5ccc6ccccc6c5)nc(-c5cccc6oc7ccccc7c56)n4)cc23)cc1 ; c1ccc(-c2ccc(-c3nc(-c4ccc5c(c4)oc4ccccc45)nc(-c4ccc5c(c4)c(-c4ccccc4)cc4oc6ccccc6c45)n3)cc2)cc1]

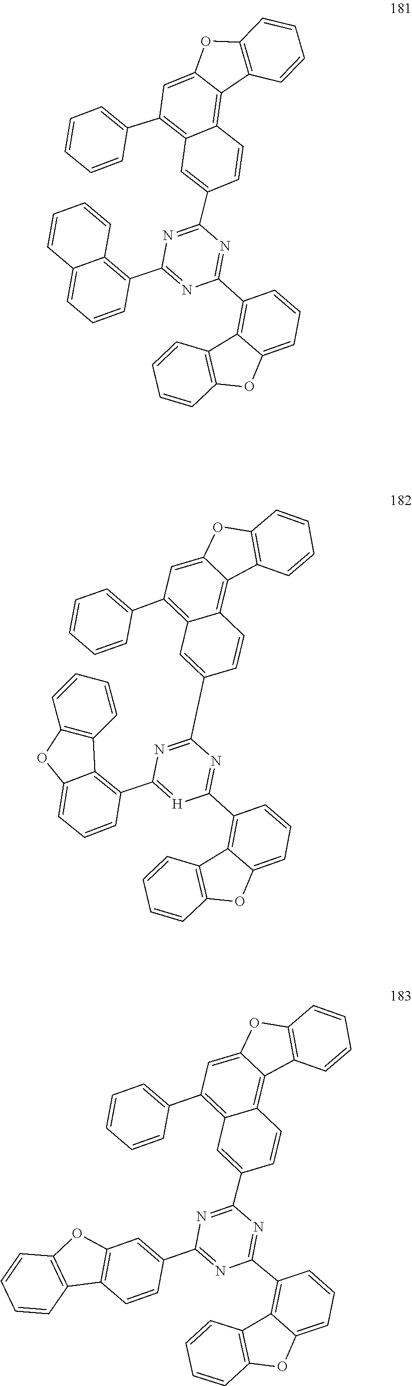

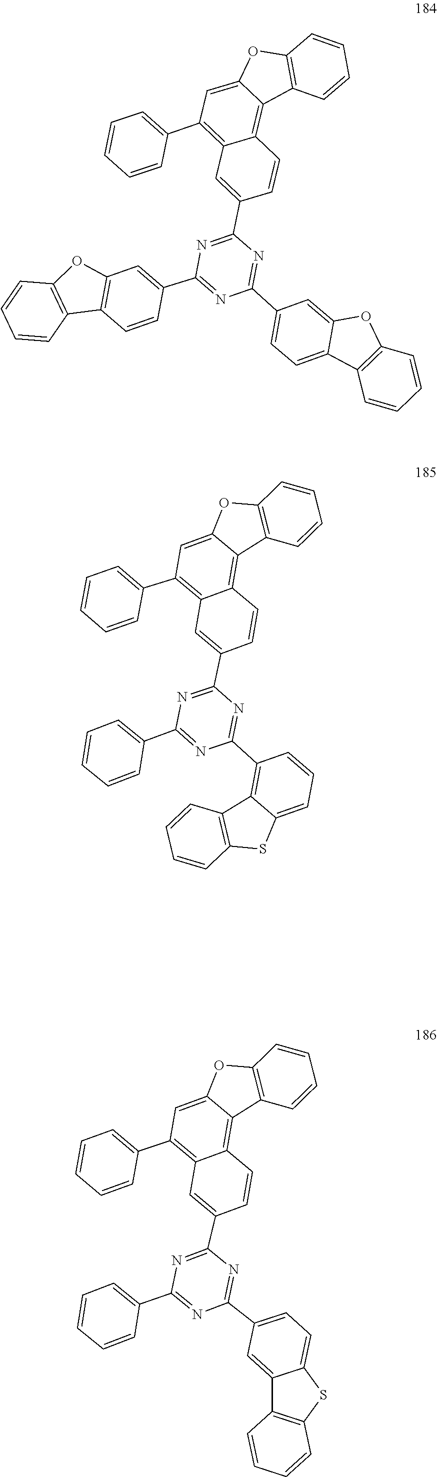 [SMILES: c1ccc(-c2cc3oc4ccccc4c3c3ccc(-c4nc(-c5ccc6c(c5)oc5ccccc56)nc(-c5ccc6c(c5)oc5ccccc56)n4)cc23)cc1 ; c1ccc(-c2nc(-c3ccc4c(c3)c(-c3ccccc3)cc3oc5ccccc5c34)nc(-c3cccc4sc5ccccc5c34)n2)cc1 ; c1ccc(-c2nc(-c3ccc4c(c3)c(-c3ccccc3)cc3oc5ccccc5c34)nc(-c3ccc4sc5ccccc5c4c3)n2)cc1]

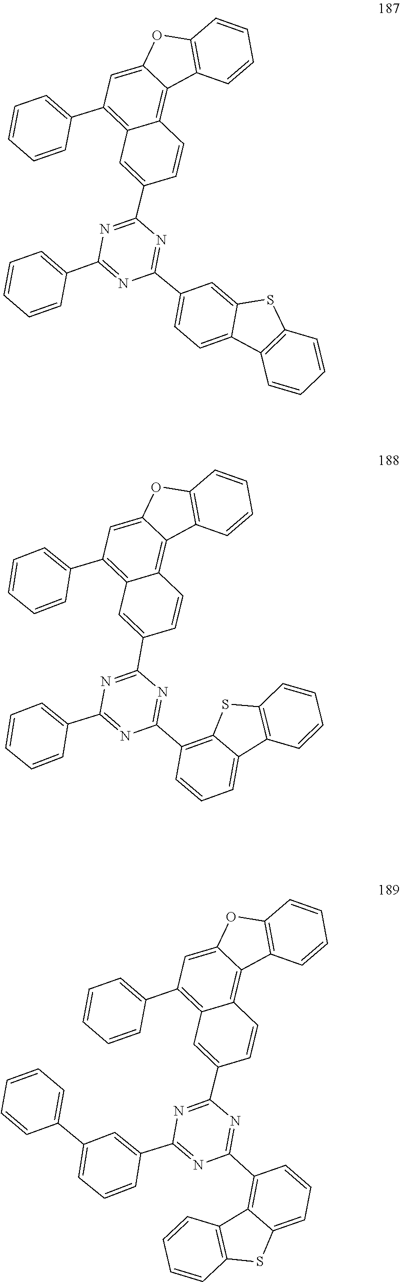 [SMILES: c1ccc(-c2nc(-c3ccc4c(c3)sc3ccccc34)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3cccc4c3sc3ccccc34)n2)cc1 ; c1ccc(-c2cccc(-c3nc(-c4ccc5c(c4)c(-c4ccccc4)cc4oc6ccccc6c45)nc(-c4cccc5sc6ccccc6c45)n3)c2)cc1]

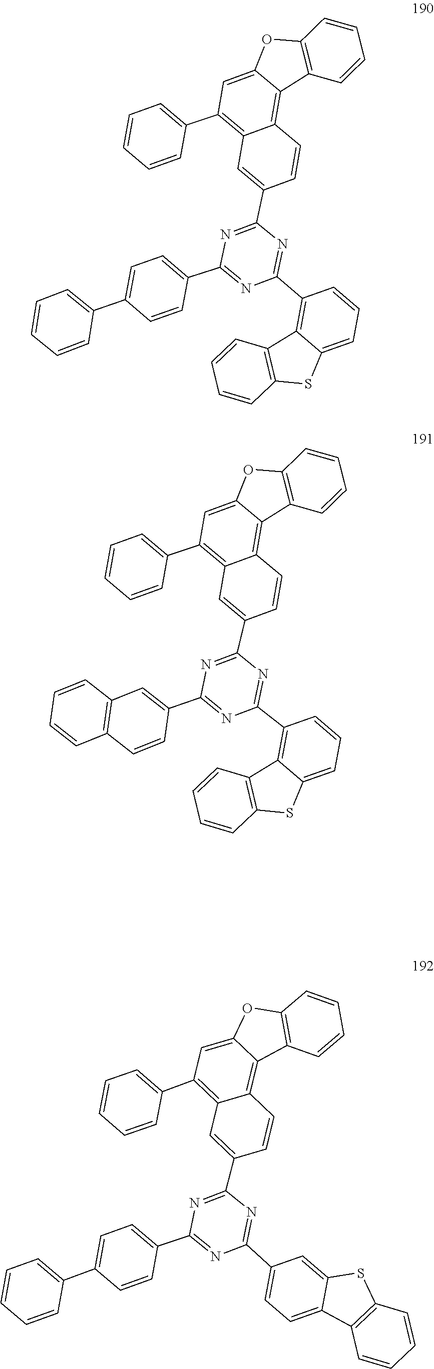 [SMILES: c1ccc(-c2ccc(-c3nc(-c4ccc5c(c4)c(-c4ccccc4)cc4oc6ccccc6c45)nc(-c4cccc5sc6ccccc6c45)n3)cc2)cc1 ; c1ccc(-c2cc3oc4ccccc4c3c3ccc(-c4nc(-c5ccc6ccccc6c5)nc(-c5cccc6sc7ccccc7c56)n4)cc23)cc1 ; c1ccc(-c2ccc(-c3nc(-c4ccc5c(c4)sc4ccccc45)nc(-c4ccc5c(c4)c(-c4ccccc4)cc4oc6ccccc6c45)n3)cc2)cc1]

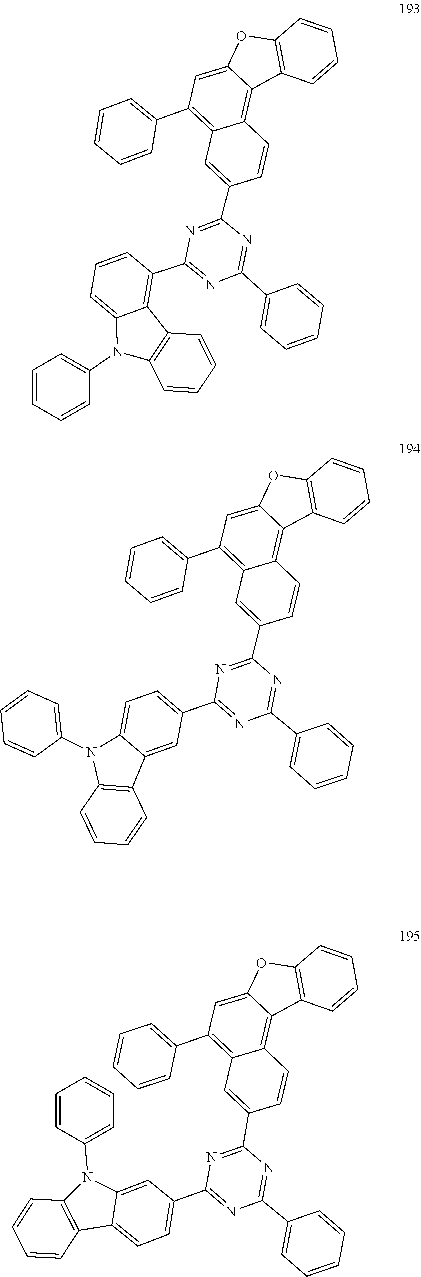 [SMILES: c1ccc(-c2nc(-c3ccc4c(c3)c(-c3ccccc3)cc3oc5ccccc5c34)nc(-c3cccc4c3c3ccccc3n4-c3ccccc3)n2)cc1 ; c1ccc(-c2nc(-c3ccc4c(c3)c(-c3ccccc3)cc3oc5ccccc5c34)nc(-c3ccc4c(c3)c3ccccc3n4-c3ccccc3)n2)cc1 ; c1ccc(-c2nc(-c3ccc4c(c3)c(-c3ccccc3)cc3oc5ccccc5c34)nc(-c3ccc4c5ccccc5n(-c5ccccc5)c4c3)n2)cc1]

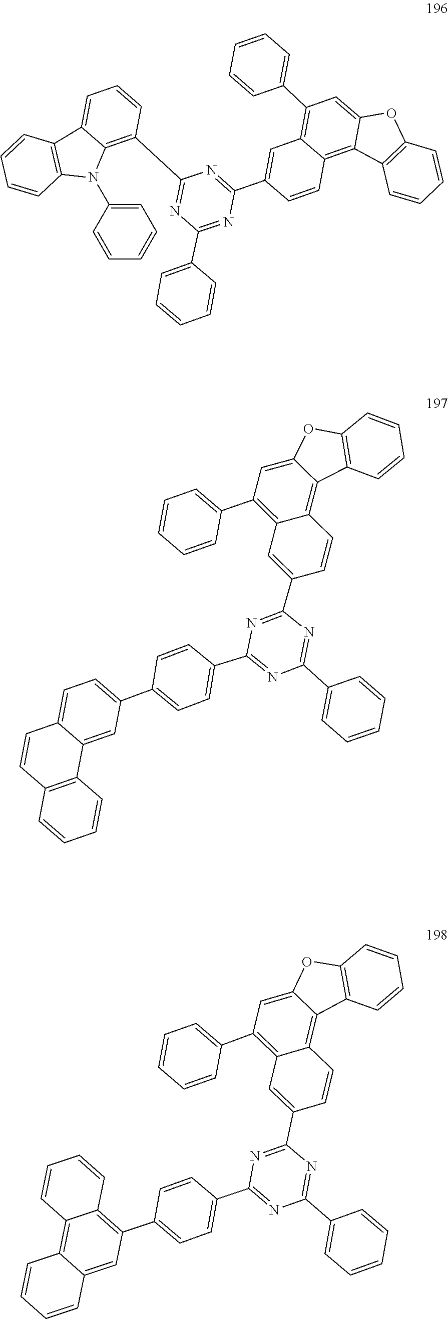 [SMILES: c1ccc(-c2nc(-c3ccc4c(c3)c(-c3ccccc3)cc3oc5ccccc5c34)nc(-c3cccc4c5ccccc5n(-c5ccccc5)c34)n2)cc1 ; c1ccc(-c2nc(-c3ccc(-c4ccc5ccc6ccccc6c5c4)cc3)nc(-c3ccc4c(c3)c(-c3ccccc3)cc3oc5ccccc5c34)n2)cc1 ; c1ccc(-c2nc(-c3ccc(-c4cc5ccccc5c5ccccc45)cc3)nc(-c3ccc4c(c3)c(-c3ccccc3)cc3oc5ccccc5c34)n2)cc1]

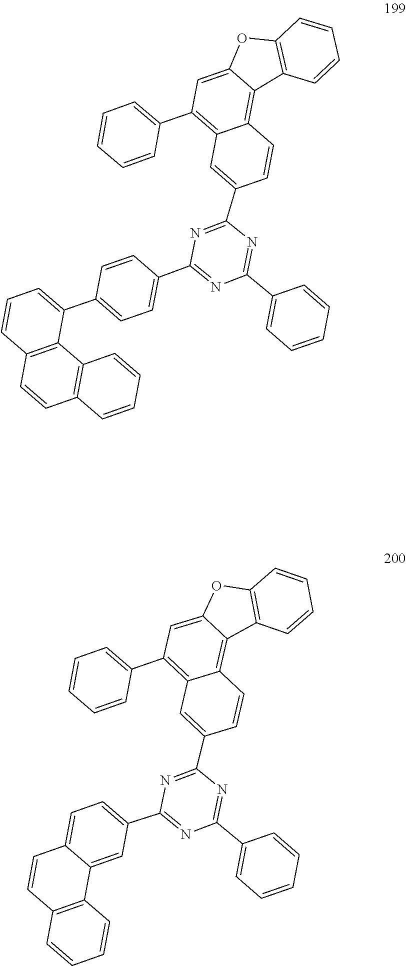 [SMILES: c1ccc(-c2nc(-c3ccc(-c4cccc5ccc6ccccc6c45)cc3)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3ccc4ccc5ccccc5c4c3)n2)cc1]

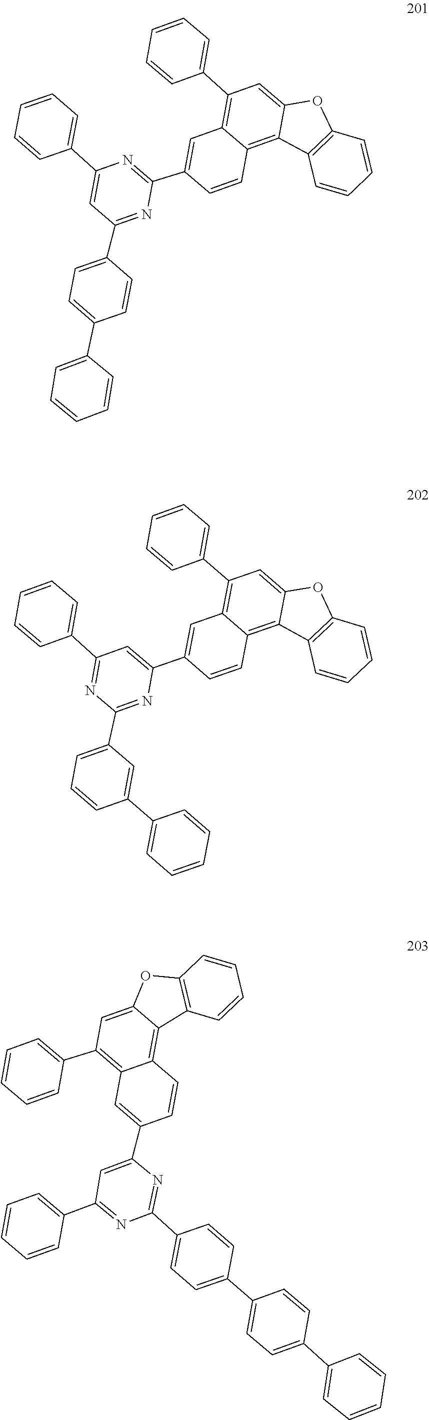 [SMILES: c1ccc(-c2ccc(-c3cc(-c4ccccc4)nc(-c4ccc5c(c4)c(-c4ccccc4)cc4oc6ccccc6c45)n3)cc2)cc1 ; c1ccc(-c2cccc(-c3nc(-c4ccccc4)cc(-c4ccc5c(c4)c(-c4ccccc4)cc4oc6ccccc6c45)n3)c2)cc1 ; c1ccc(-c2ccc(-c3ccc(-c4nc(-c5ccccc5)cc(-c5ccc6c(c5)c(-c5ccccc5)cc5oc7ccccc7c56)n4)cc3)cc2)cc1]

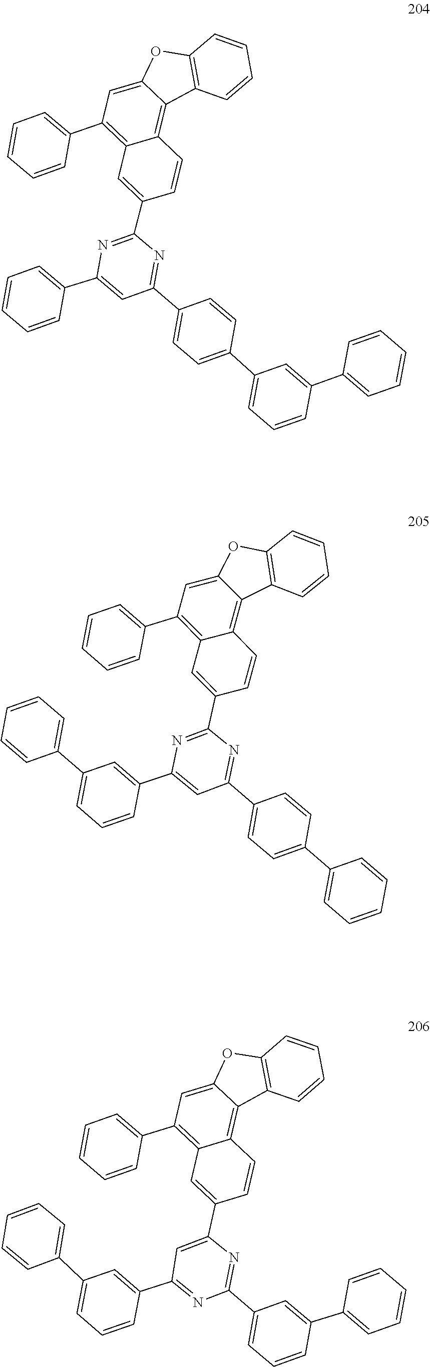 [SMILES: c1ccc(-c2cccc(-c3ccc(-c4cc(-c5ccccc5)nc(-c5ccc6c(c5)c(-c5ccccc5)cc5oc7ccccc7c56)n4)cc3)c2)cc1 ; c1ccc(-c2ccc(-c3cc(-c4cccc(-c5ccccc5)c4)nc(-c4ccc5c(c4)c(-c4ccccc4)cc4oc6ccccc6c45)n3)cc2)cc1 ; c1ccc(-c2cccc(-c3cc(-c4ccc5c(c4)c(-c4ccccc4)cc4oc6ccccc6c45)nc(-c4cccc(-c5ccccc5)c4)n3)c2)cc1]

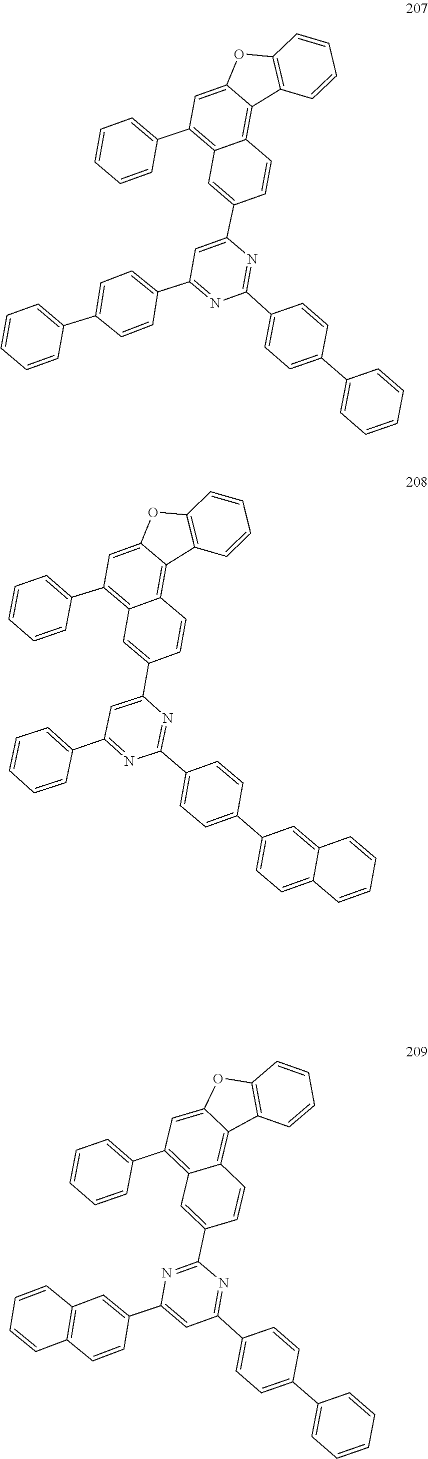 [SMILES: c1ccc(-c2ccc(-c3cc(-c4ccc5c(c4)c(-c4ccccc4)cc4oc6ccccc6c45)nc(-c4ccc(-c5ccccc5)cc4)n3)cc2)cc1 ; c1ccc(-c2cc(-c3ccc4c(c3)c(-c3ccccc3)cc3oc5ccccc5c34)nc(-c3ccc(-c4ccc5ccccc5c4)cc3)n2)cc1 ; c1ccc(-c2ccc(-c3cc(-c4ccc5ccccc5c4)nc(-c4ccc5c(c4)c(-c4ccccc4)cc4oc6ccccc6c45)n3)cc2)cc1]

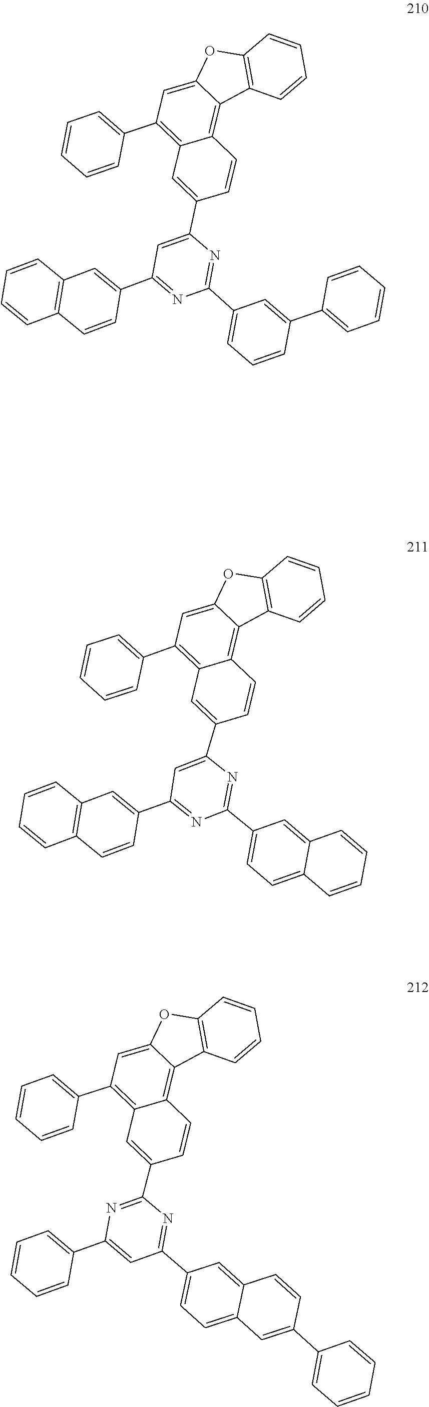 [SMILES: c1ccc(-c2cccc(-c3nc(-c4ccc5ccccc5c4)cc(-c4ccc5c(c4)c(-c4ccccc4)cc4oc6ccccc6c45)n3)c2)cc1 ; c1ccc(-c2cc3oc4ccccc4c3c3ccc(-c4cc(-c5ccc6ccccc6c5)nc(-c5ccc6ccccc6c5)n4)cc23)cc1 ; c1ccc(-c2ccc3cc(-c4cc(-c5ccccc5)nc(-c5ccc6c(c5)c(-c5ccccc5)cc5oc7ccccc7c56)n4)ccc3c2)cc1]

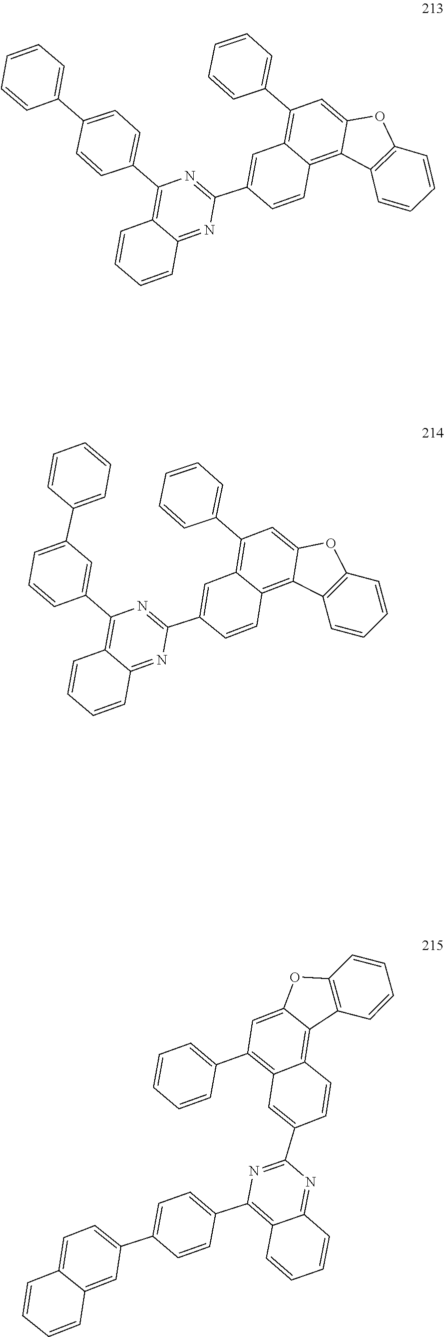 [SMILES: c1ccc(-c2ccc(-c3nc(-c4ccc5c(c4)c(-c4ccccc4)cc4oc6ccccc6c45)nc4ccccc34)cc2)cc1 ; c1ccc(-c2cccc(-c3nc(-c4ccc5c(c4)c(-c4ccccc4)cc4oc6ccccc6c45)nc4ccccc34)c2)cc1 ; c1ccc(-c2cc3oc4ccccc4c3c3ccc(-c4nc(-c5ccc(-c6ccc7ccccc7c6)cc5)c5ccccc5n4)cc23)cc1]

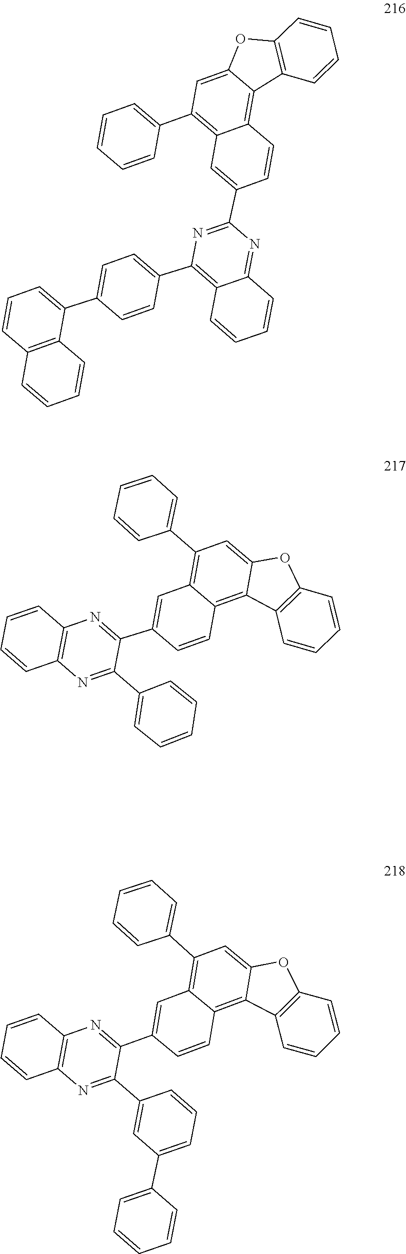 [SMILES: c1ccc(-c2cc3oc4ccccc4c3c3ccc(-c4nc(-c5ccc(-c6cccc7ccccc67)cc5)c5ccccc5n4)cc23)cc1 ; c1ccc(-c2nc3ccccc3nc2-c2ccc3c(c2)c(-c2ccccc2)cc2oc4ccccc4c23)cc1 ; c1ccc(-c2cccc(-c3nc4ccccc4nc3-c3ccc4c(c3)c(-c3ccccc3)cc3oc5ccccc5c34)c2)cc1]

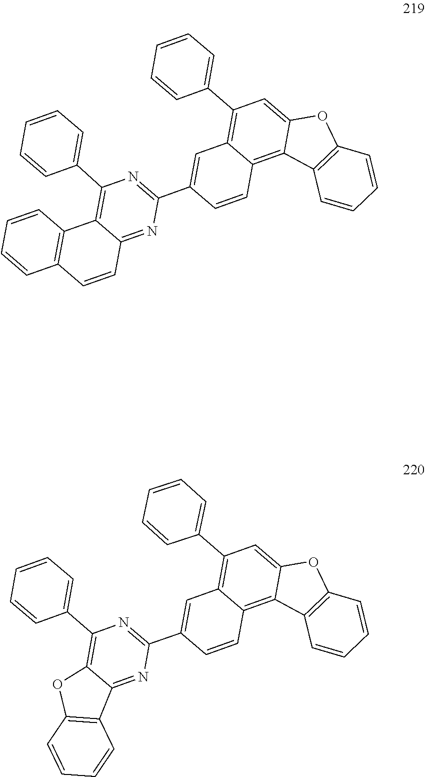 [SMILES: c1ccc(-c2cc3oc4ccccc4c3c3ccc(-c4nc(-c5ccccc5)c5c(ccc6ccccc65)n4)cc23)cc1 ; c1ccc(-c2cc3oc4ccccc4c3c3ccc(-c4nc(-c5ccccc5)c5oc6ccccc6c5n4)cc23)cc1]

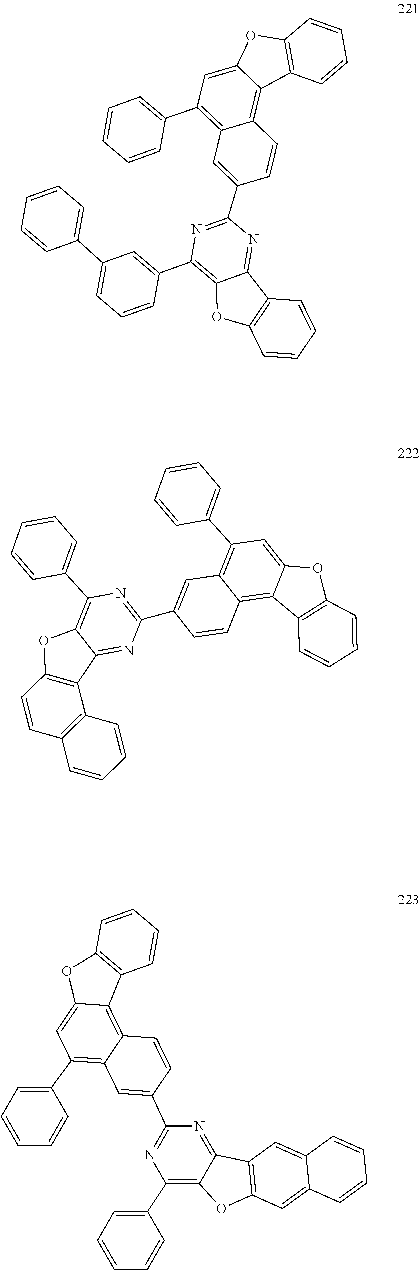 [SMILES: c1ccc(-c2cccc(-c3nc(-c4ccc5c(c4)c(-c4ccccc4)cc4oc6ccccc6c45)nc4c3oc3ccccc34)c2)cc1 ; c1ccc(-c2cc3oc4ccccc4c3c3ccc(-c4nc(-c5ccccc5)c5oc6ccc7ccccc7c6c5n4)cc23)cc1 ; c1ccc(-c2cc3oc4ccccc4c3c3ccc(-c4nc(-c5ccccc5)c5oc6cc7ccccc7cc6c5n4)cc23)cc1]

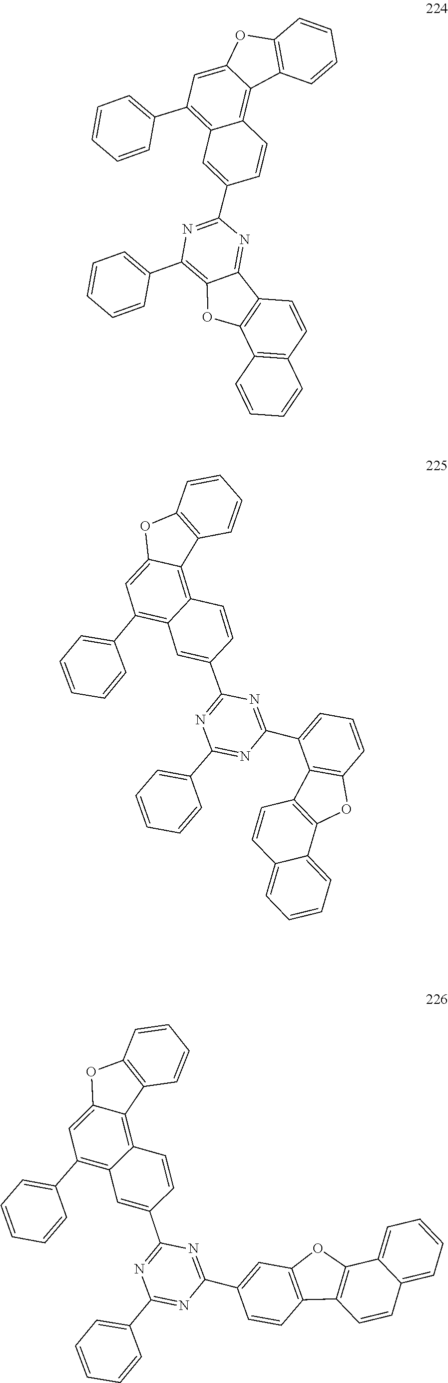 [SMILES: c1ccc(-c2cc3oc4ccccc4c3c3ccc(-c4nc(-c5ccccc5)c5oc6c7ccccc7ccc6c5n4)cc23)cc1 ; c1ccc(-c2nc(-c3ccc4c(c3)c(-c3ccccc3)cc3oc5ccccc5c34)nc(-c3cccc4oc5c6ccccc6ccc5c34)n2)cc1 ; c1ccc(-c2nc(-c3ccc4c(c3)oc3c5ccccc5ccc43)nc(-c3ccc4c(c3)c(-c3ccccc3)cc3oc5ccccc5c34)n2)cc1]

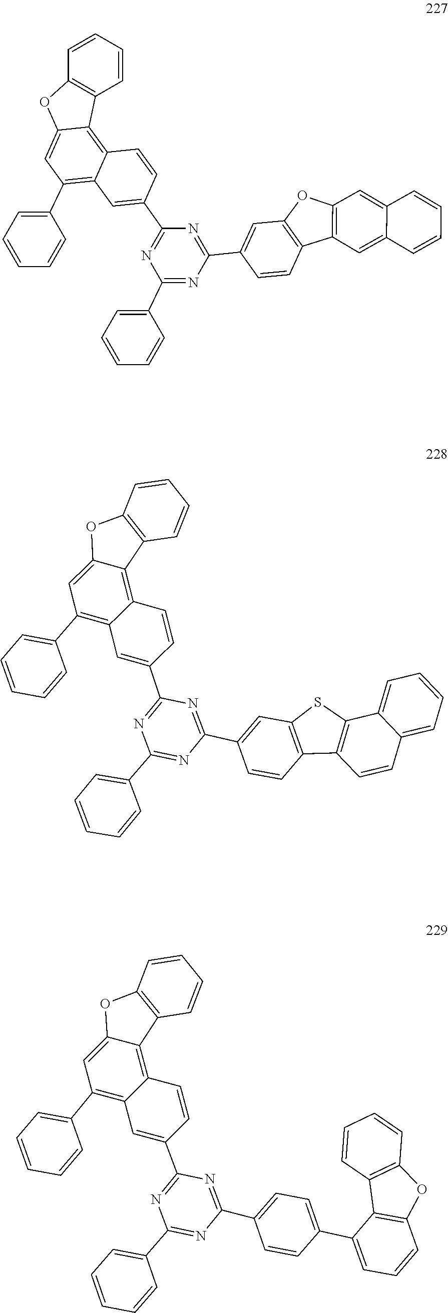 [SMILES: c1ccc(-c2nc(-c3ccc4c(c3)oc3cc5ccccc5cc34)nc(-c3ccc4c(c3)c(-c3ccccc3)cc3oc5ccccc5c34)n2)cc1 ; c1ccc(-c2nc(-c3ccc4c(c3)sc3c5ccccc5ccc43)nc(-c3ccc4c(c3)c(-c3ccccc3)cc3oc5ccccc5c34)n2)cc1 ; c1ccc(-c2nc(-c3ccc(-c4cccc5oc6ccccc6c45)cc3)nc(-c3ccc4c(c3)c(-c3ccccc3)cc3oc5ccccc5c34)n2)cc1]

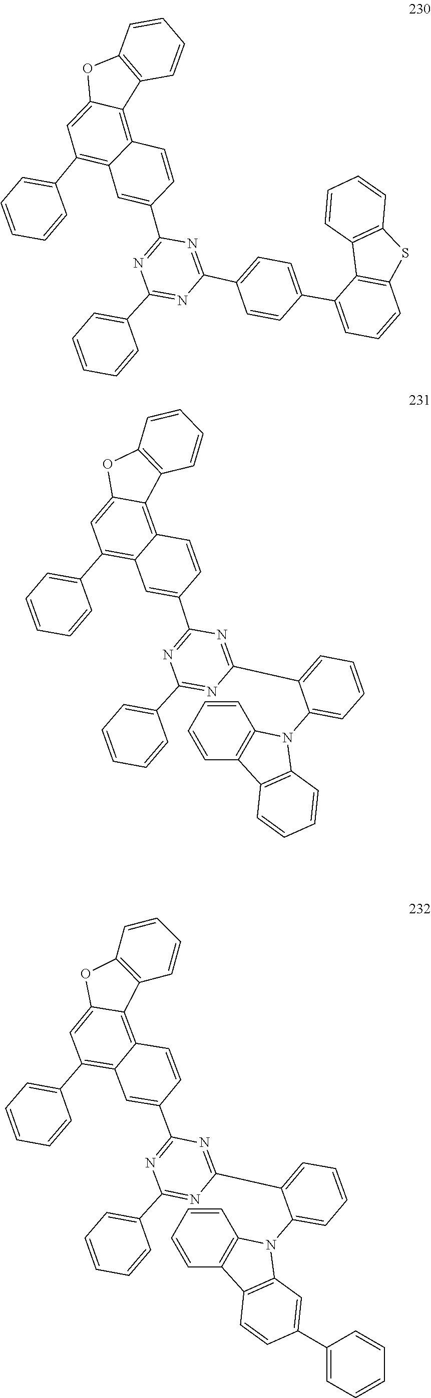 [SMILES: c1ccc(-c2nc(-c3ccc(-c4cccc5sc6ccccc6c45)cc3)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3ccccc3-n3c4ccccc4c4ccccc43)n2)cc1 ; c1ccc(-c2ccc3c4ccccc4n(-c4ccccc4-c4nc(-c5ccccc5)nc(-c5ccc6c(c5)c(-c5ccccc5)cc5oc7ccccc7c56)n4)c3c2)cc1]

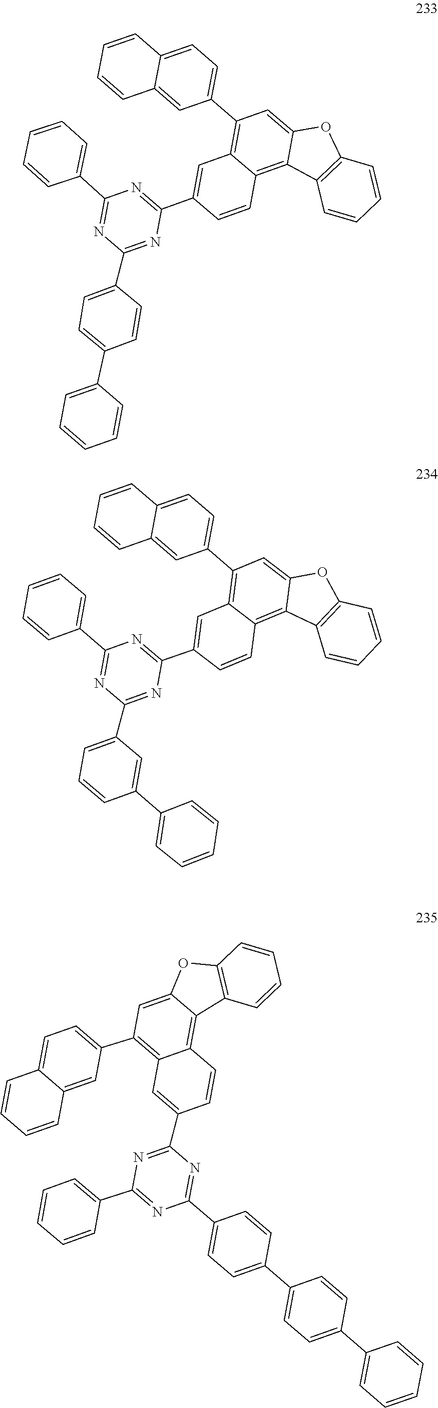 [SMILES: c1ccc(-c2ccc(-c3nc(-c4ccccc4)nc(-c4ccc5c(c4)c(-c4ccc6ccccc6c4)cc4oc6ccccc6c45)n3)cc2)cc1 ; c1ccc(-c2cccc(-c3nc(-c4ccccc4)nc(-c4ccc5c(c4)c(-c4ccc6ccccc6c4)cc4oc6ccccc6c45)n3)c2)cc1 ; c1ccc(-c2ccc(-c3ccc(-c4nc(-c5ccccc5)nc(-c5ccc6c(c5)c(-c5ccc7ccccc7c5)cc5oc7ccccc7c56)n4)cc3)cc2)cc1]

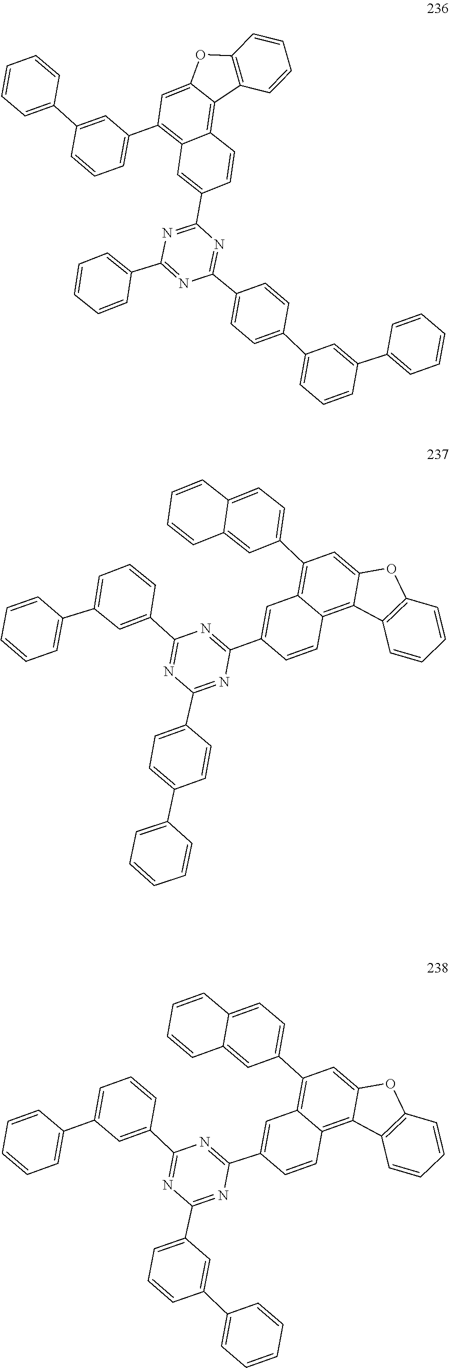 [SMILES: c1ccc(-c2cccc(-c3ccc(-c4nc(-c5ccccc5)nc(-c5ccc6c(c5)c(-c5cccc(-c7ccccc7)c5)cc5oc7ccccc7c56)n4)cc3)c2)cc1 ; c1ccc(-c2ccc(-c3nc(-c4cccc(-c5ccccc5)c4)nc(-c4ccc5c(c4)c(-c4ccc6ccccc6c4)cc4oc6ccccc6c45)n3)cc2)cc1 ; c1ccc(-c2cccc(-c3nc(-c4cccc(-c5ccccc5)c4)nc(-c4ccc5c(c4)c(-c4ccc6ccccc6c4)cc4oc6ccccc6c45)n3)c2)cc1]

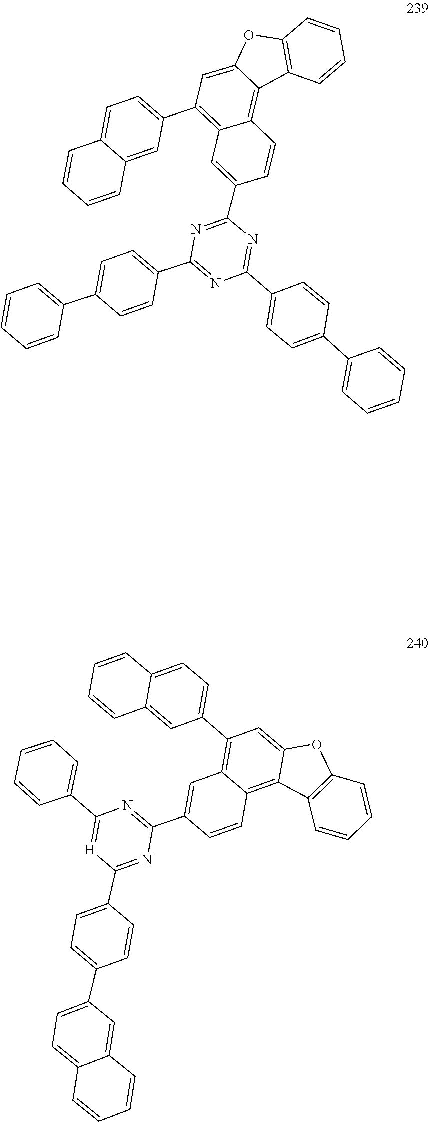

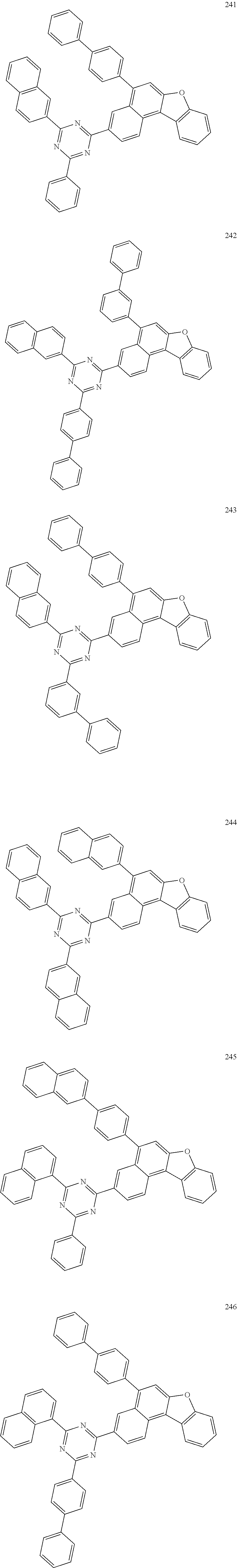

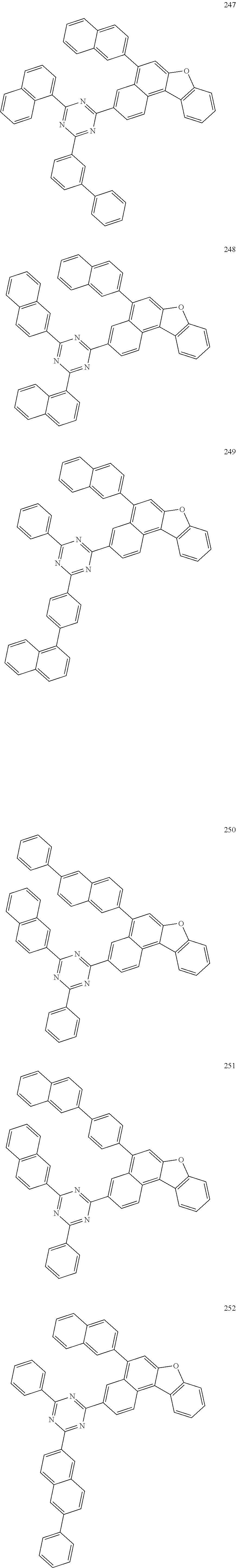

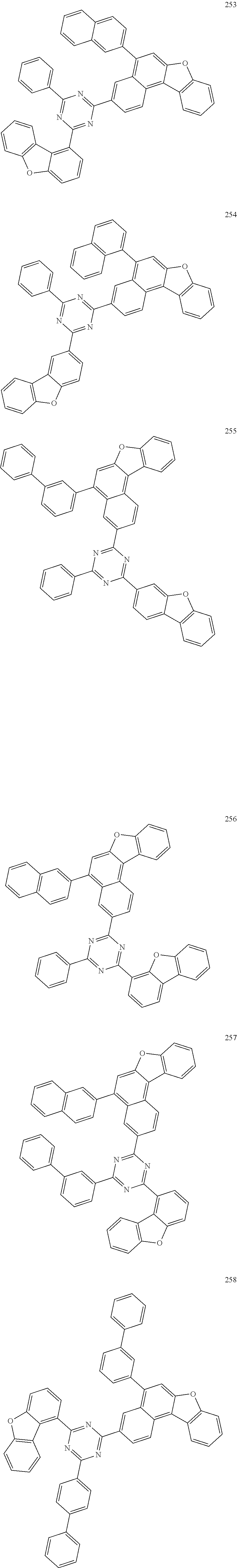

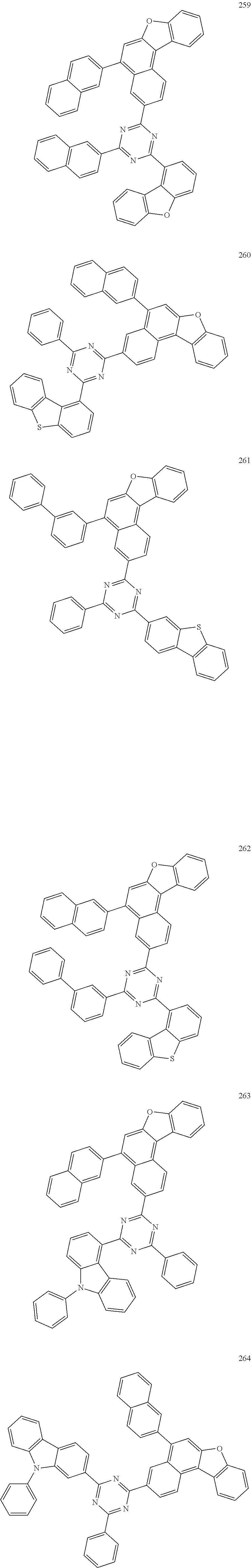

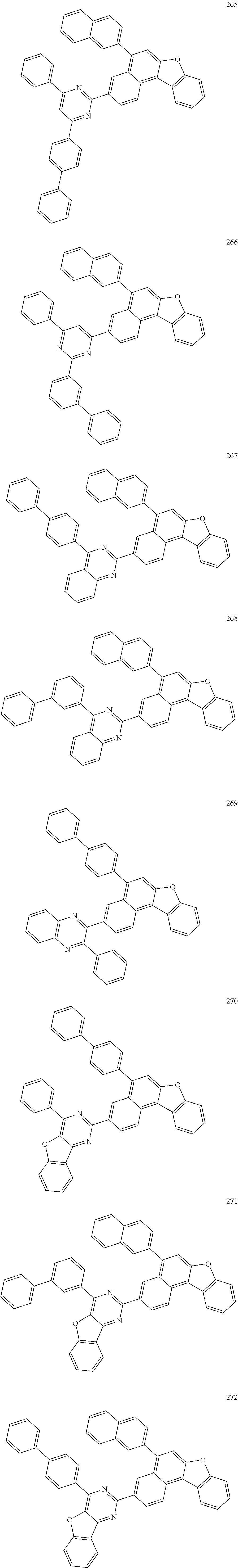

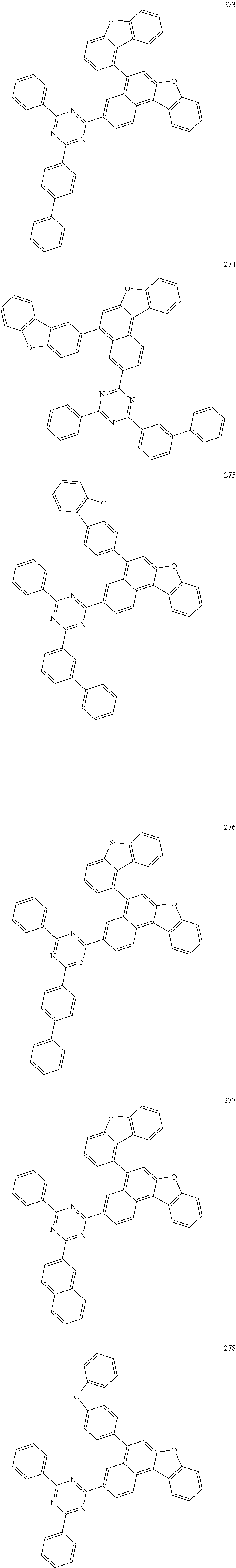

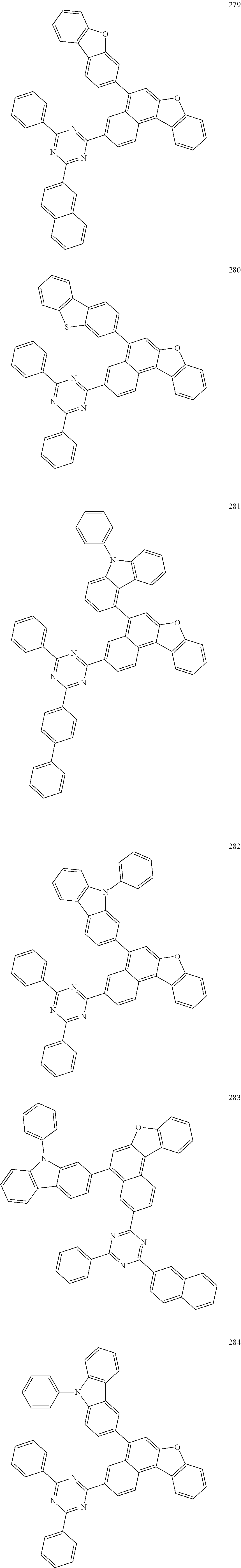

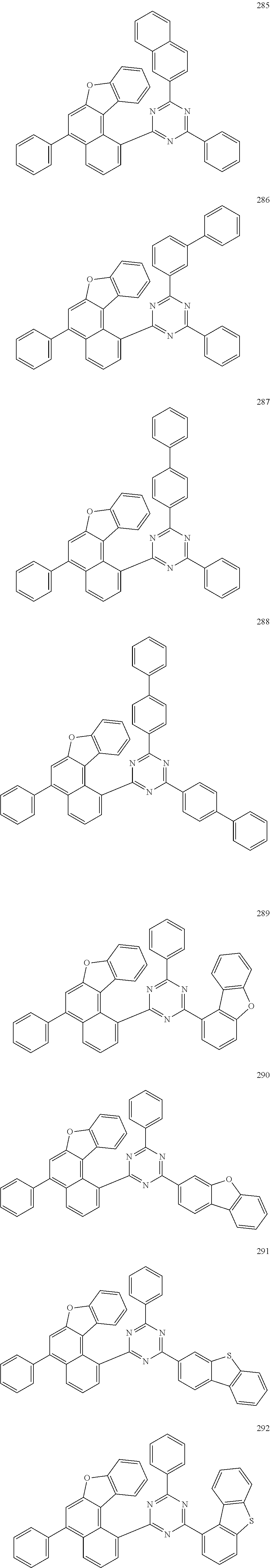

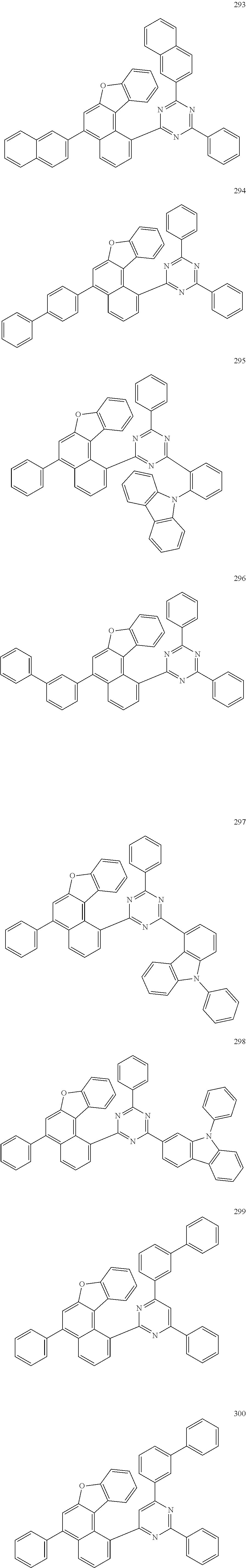

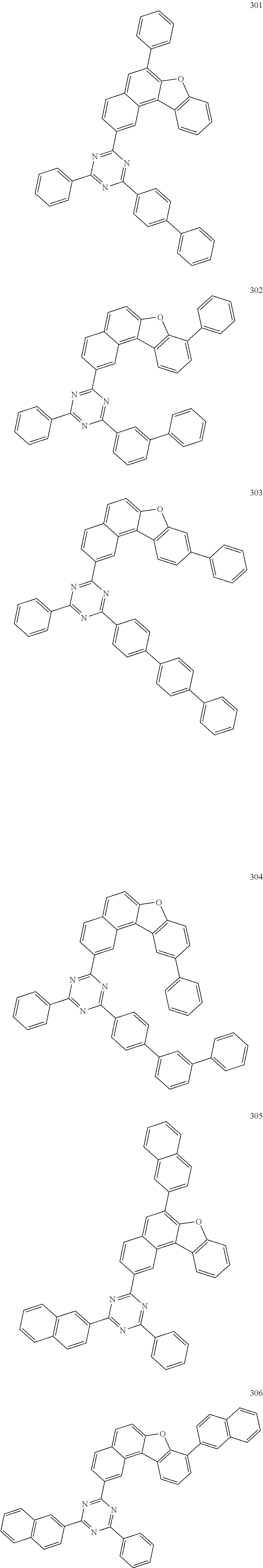

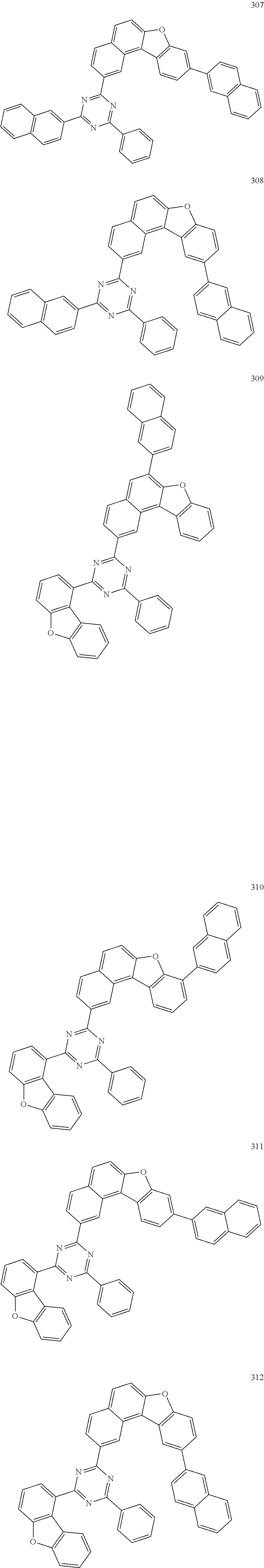

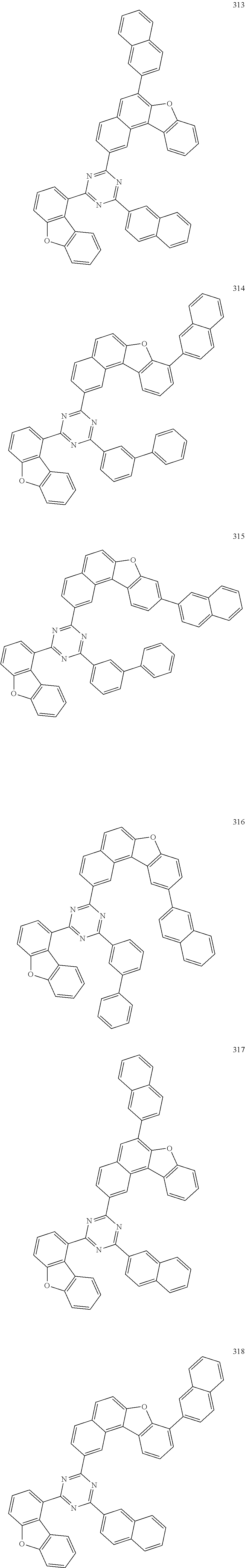

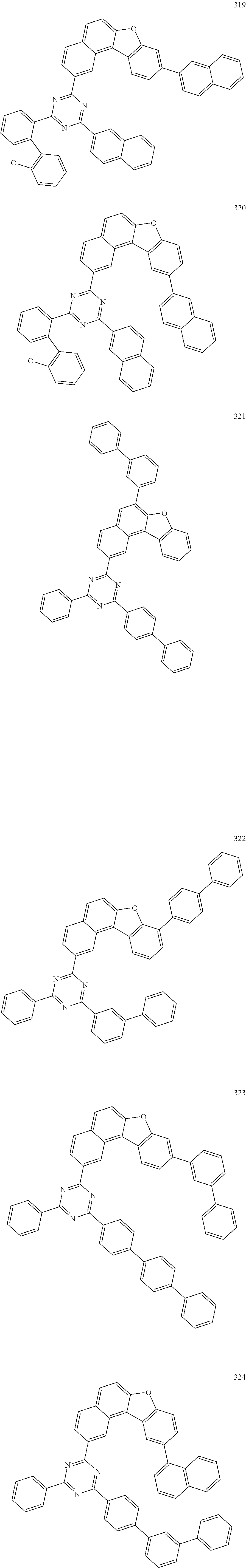

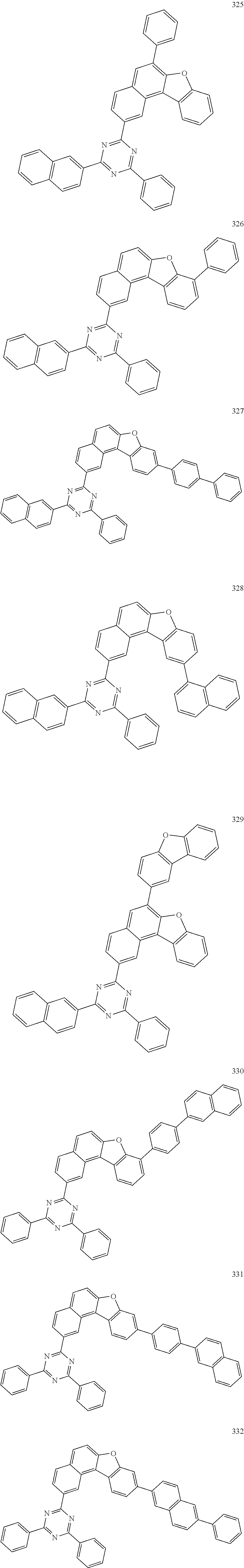

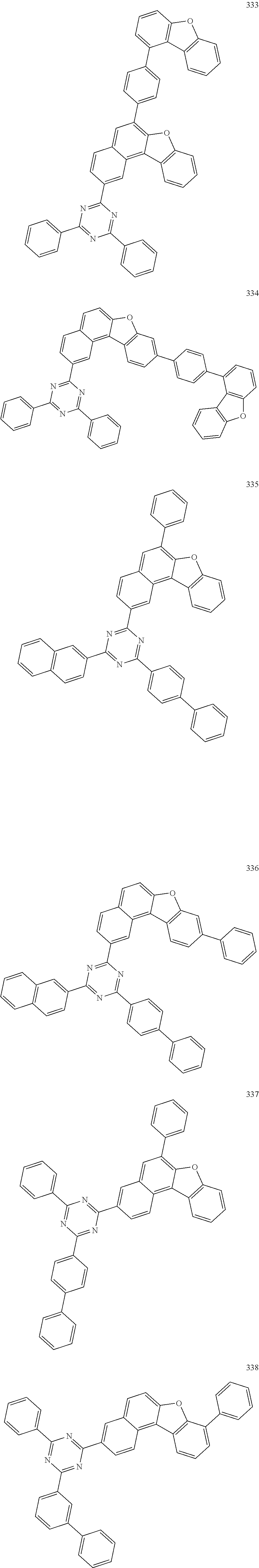

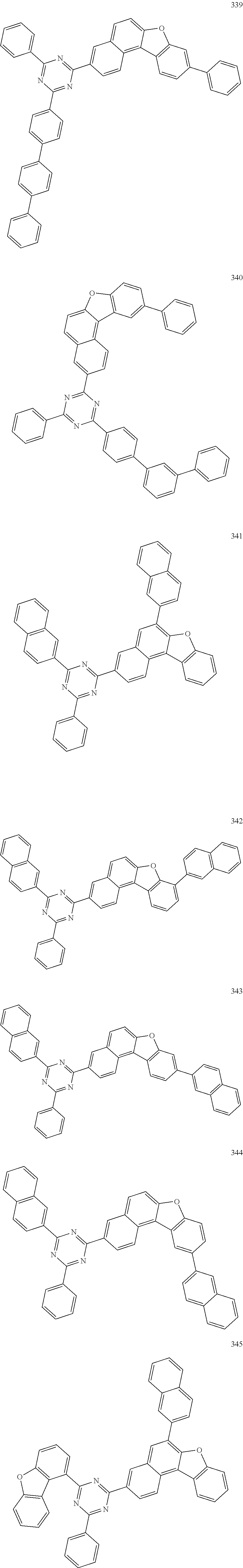

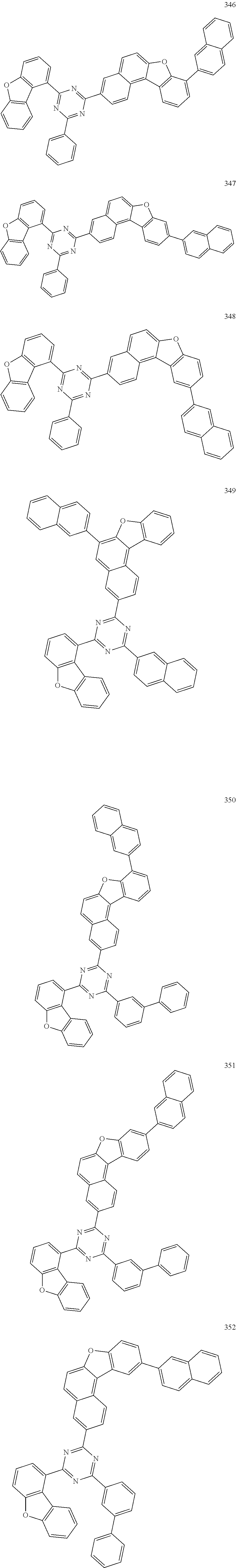

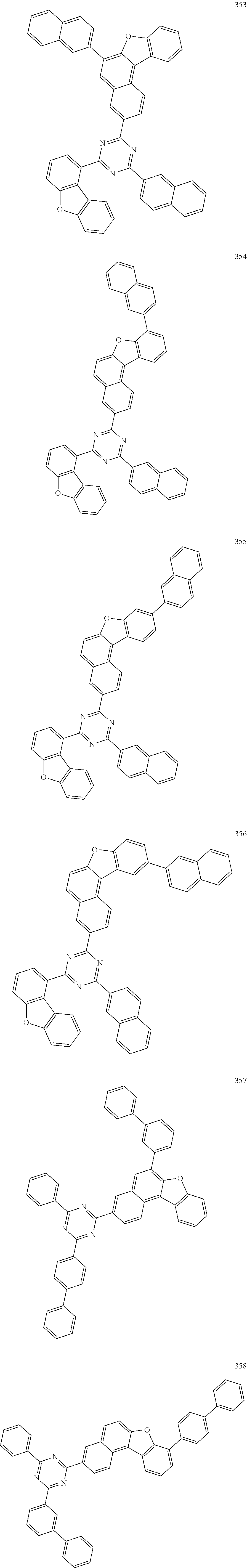

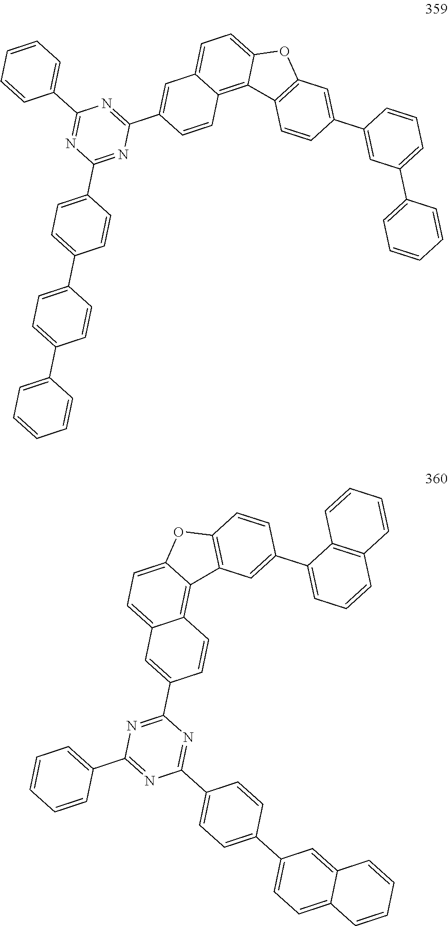 [SMILES: c1ccc(-c2ccc(-c3ccc(-c4nc(-c5ccccc5)nc(-c5ccc6c(ccc7oc8cc(-c9cccc(-c%10ccccc%10)c9)ccc8c76)c5)n4)cc3)cc2)cc1 ; c1ccc(-c2nc(-c3ccc(-c4ccc5ccccc5c4)cc3)nc(-c3ccc4c(ccc5oc6ccc(-c7cccc8ccccc78)cc6c54)c3)n2)cc1]

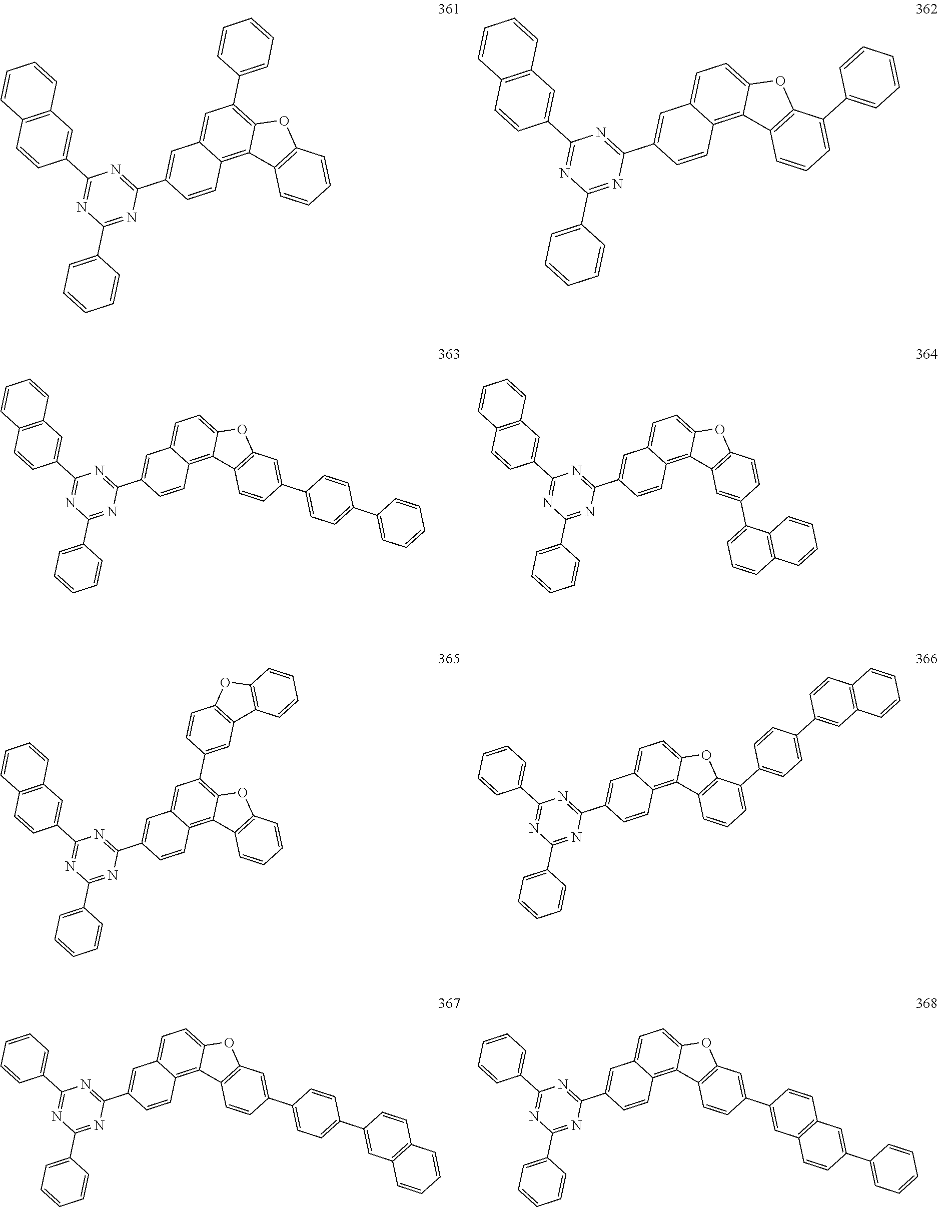

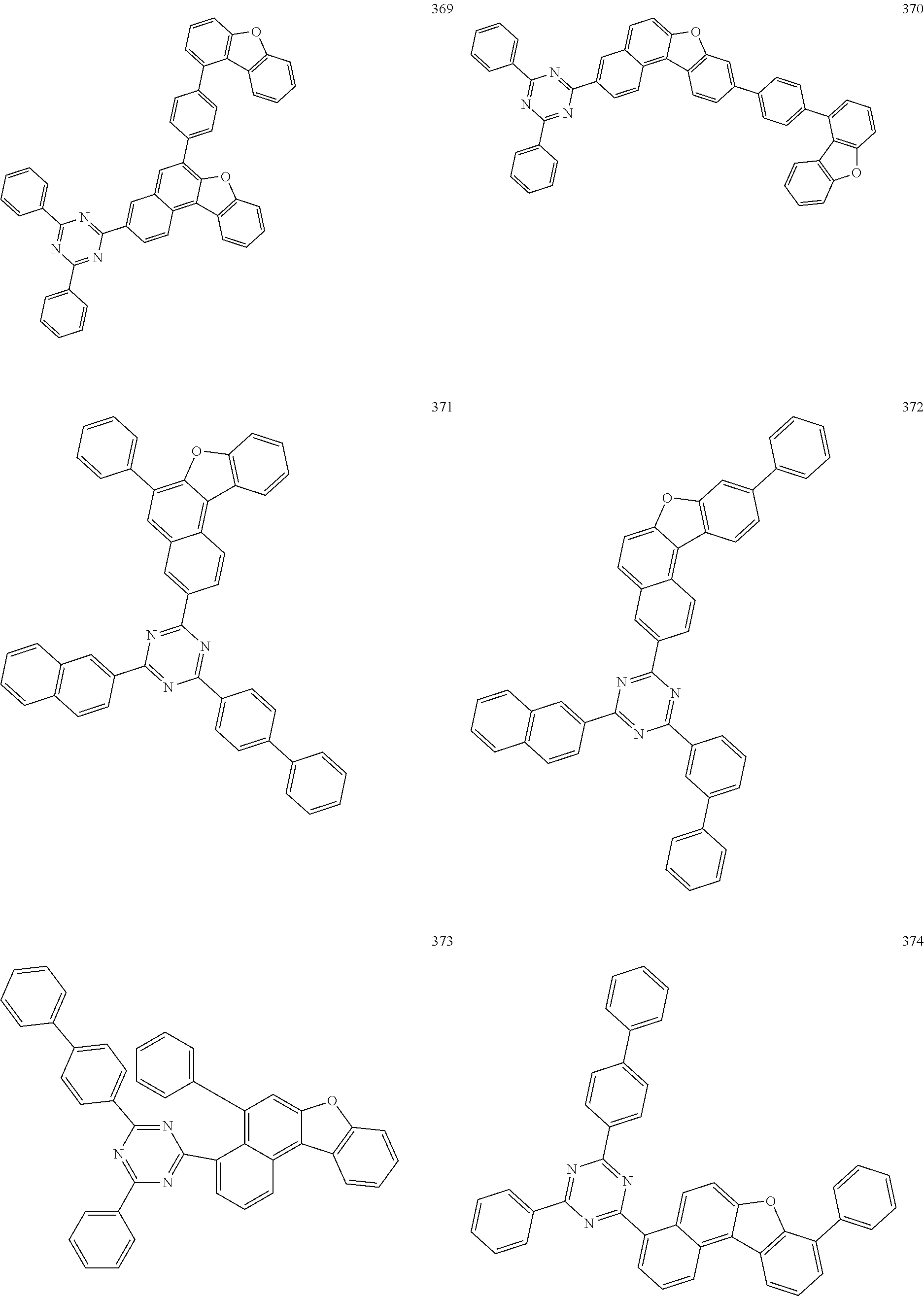

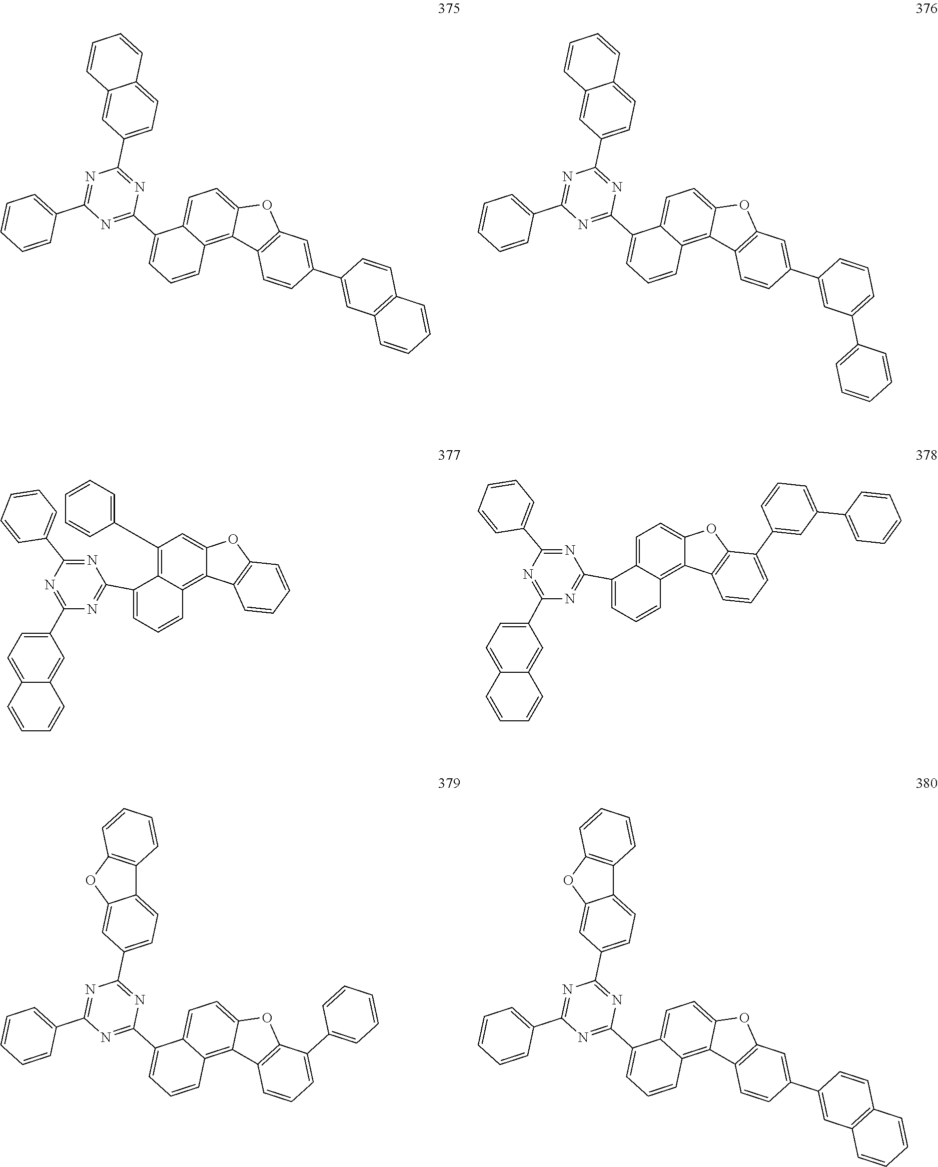

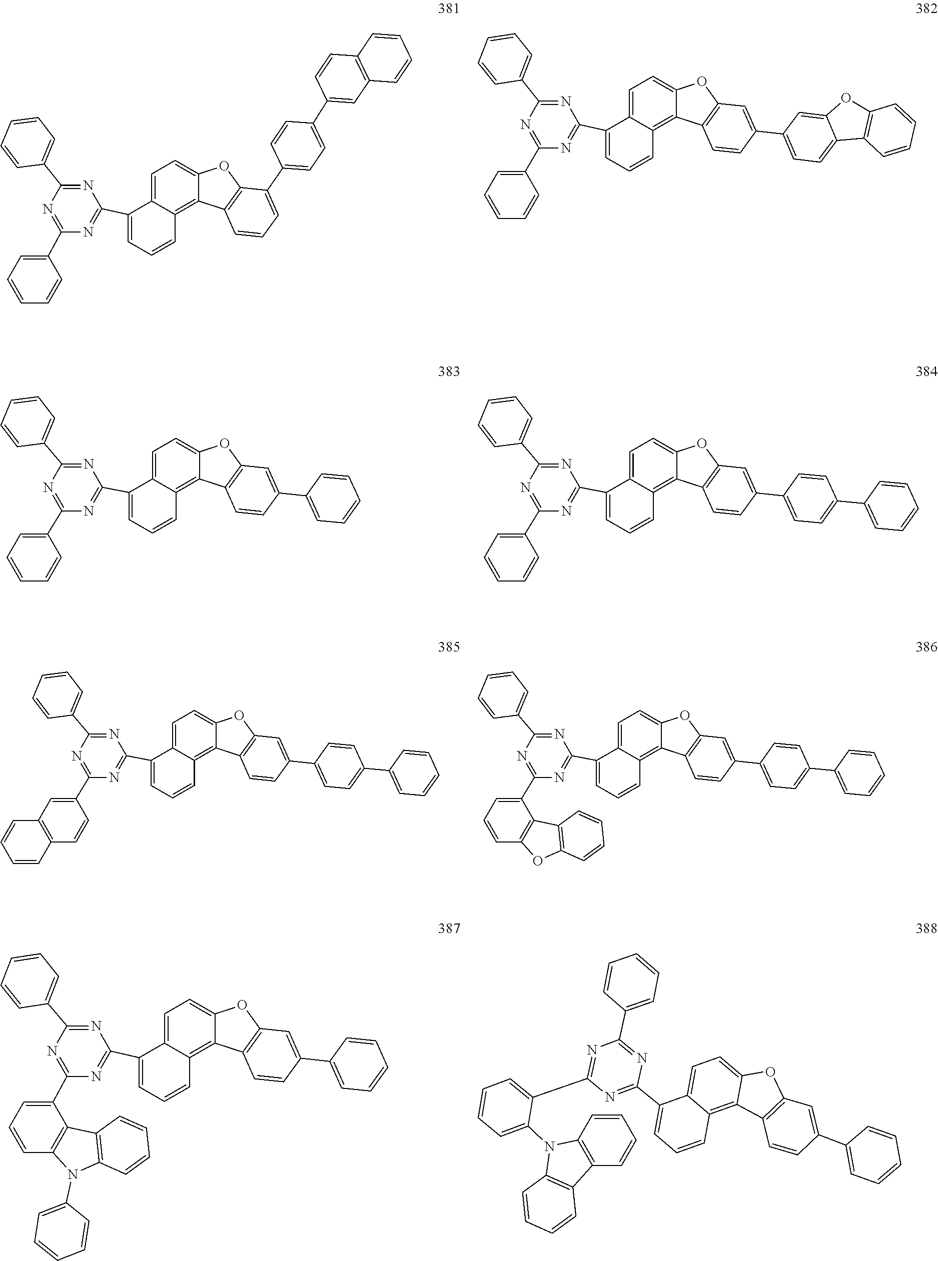

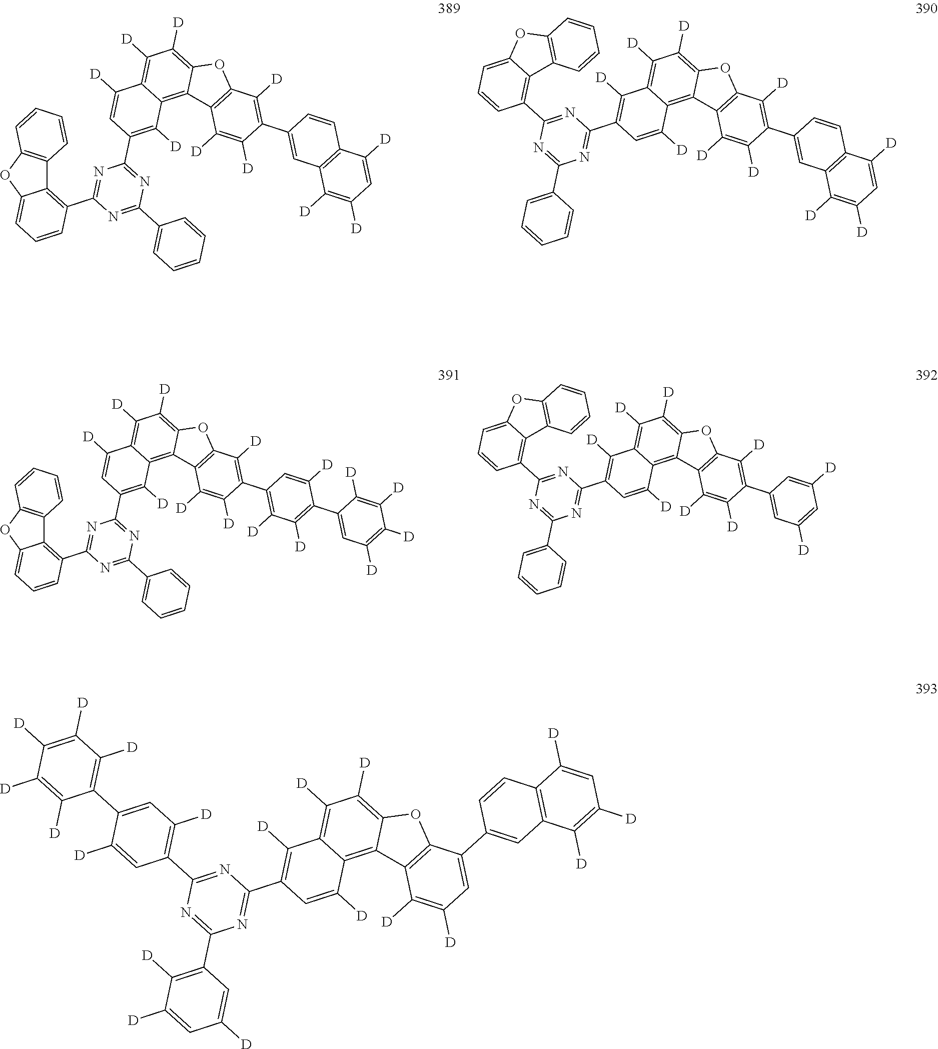

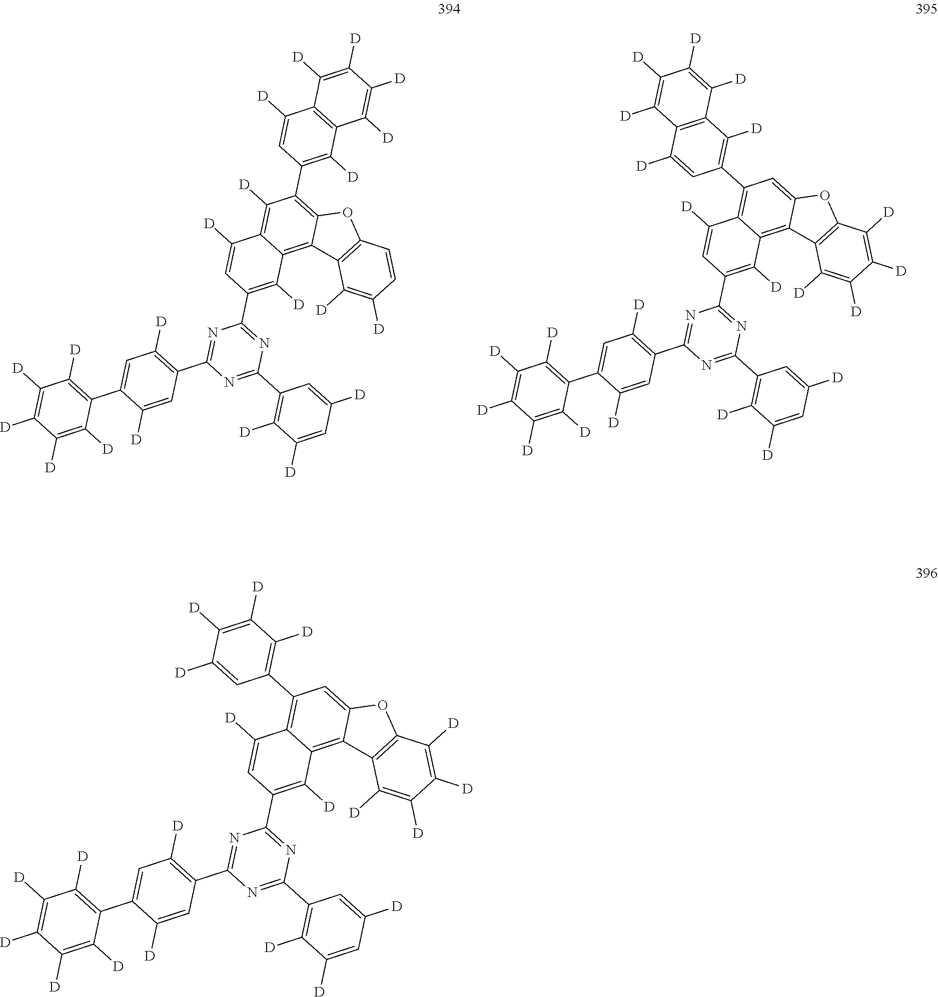

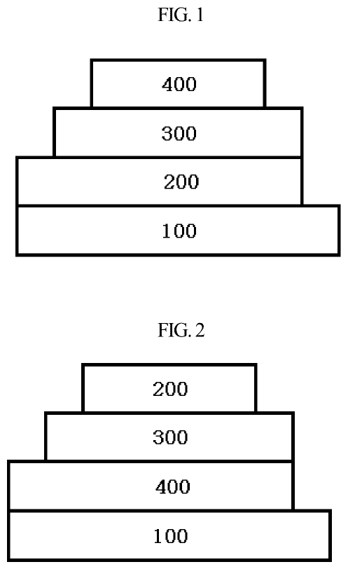

FIGS. 1 to 4 illustrate the stacking order of electrodes and organic layers of a light-emitting device according to an embodiment of the present application. However, the scope of the present application is not intended to be limited to these drawings, and the structure of organic light-emitting devices known in the art may also be applied to the present application.

Referring to FIG. 1, a light-emitting device is illustrated, in which a first electrode (anode, 200), an organic layer 300, and a second electrode (cathode, 400) are sequentially stacked on a substrate 100. However, it is not limited to this structure, and as shown in FIG. 2, an organic light-emitting device may be implemented in which the cathode 400, the organic layer 300, and the anode 200 are sequentially stacked on the substrate.

In an exemplary embodiment, the first electrode may be an anode, and the second electrode may be a cathode. Alternatively, the first electrode may be a cathode, and the second electrode may be an anode.

According to exemplary embodiments, materials with a relatively high work function may be used as the anode material, such as transparent conductive oxides, metals, or conductive polymers. Examples of the anode material may include metals such as vanadium, chromium, copper, zinc, and gold, or an alloy thereof, metal oxides such as zinc oxide, indium oxide, indium tin oxide (ITO), and indium zinc oxide (IZO); combinations of metals and oxides such as ZnO:Al or SnO₂:Sb; and conductive polymers such as poly(3-methylthiophene), poly[3,4(ethylene-1,2-dioxy)thiophene](PEDOT), polypyrrole and polyaniline, but are not limited thereto.

Materials with a relatively low work function may be used as cathode materials, such as metals, metal oxides, or conductive polymers. Examples of cathode materials may include metals such as magnesium, calcium, sodium, potassium, titanium, indium, yttrium, lithium, gadolinium, aluminum, silver, tin and Lead, or their alloys; and multilayer materials such as LiF/Al or LiO₂/Al, but are not limited thereto.

The light-emitting device may include one or more organic layers 300. Each of the plurality of organic layers 300 may independently have a single-layer or multilayer structure.

In an exemplary embodiment, at least one of the organic layers 300 may include a heterocyclic compound represented by Formula 1 above.

In an exemplary embodiment, at least one of the organic layers 300 may include two or more heterocyclic compounds represented by Formula 1 above.

As used herein, the specific details of the heterocyclic compound represented by Formula 1 are the same as those described above.

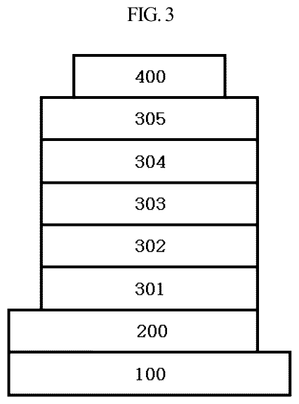

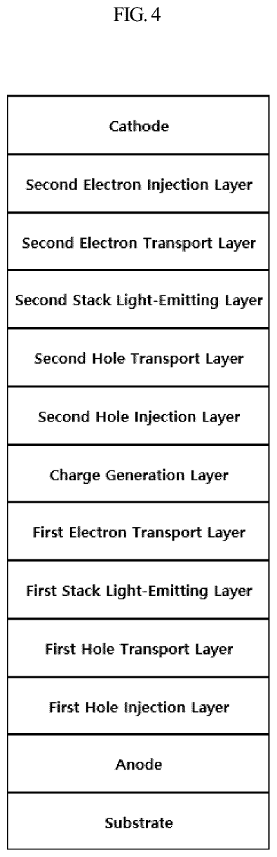

FIGS. 3 and 4 are cross-sectional views illustrating exemplary light-emitting devices in which the organic layer has a multilayer structure.

The light-emitting device shown in FIG. 3 may include a hole injection layer 301, a hole transport layer 302, a light-emitting layer 303, an electron transport layer 304, and an electron injection layer 305. However, the scope of the present application is not limited to this stacked structure and Layers other than the light-emitting layer may be omitted, and other functional layers may be added.

The light-emitting layer 303 may include a host material that is excited by holes and electrons, and a dopant material that increases light-emitting efficiency through energy absorption and emission.

In one embodiment, the light-emitting layer 303 may be independently patterned for each red light-emitting device (Pr), green light-emitting device (Pg), and blue light-emitting device (Pb), thereby emitting different colors of light for each element. For example, the light-emitting layer 303 may be patterned into a red light-emitting layer, a green light-emitting layer, and a blue light-emitting layer for each element.

In one embodiment, the light-emitting layer 303 may not be patterned for each light-emitting device, but may be provided in common to a plurality of light-emitting devices. For example, the light-emitting layer 303 may emit white light, and the color of each element may be implemented through a color filter.

The host material may include a phosphorescent host, a fluorescent host, or a combination thereof. For example, the host material may include ADN (9,10-di(2-naphthyl) anthracene), MADN (2-methyl-9,10-bis(naphthalen-2-yl) anthracene), TBADN (9,10-di-(2-naphthyl)-2-t-butyl-anthracene), CBP (4,4′-bis(N-carbazolyl)-1,1′-biphenyl), mCP (1,3-di-9-carbazolylbenzene), TCP (1,3,5-tri (carbazol-9-yl)benzene), DPEPO (bis[2-(diphenylphosphino)phenyl]ether oxide), PPF (2,8-bis(diphenylphosphoryl)dibenzo[b,d]furan), TCTA (4,4′, 4″-tris(carbazol-9-yl)-triphenylamine), CP1 (hexaphenylcyclotriphosphazene), UGH2 (1,4-bis(triphenylsilyl)benzene), DPSiO₃ (hexaphenylcyclotrisiloxane), DPSiO₄ (octaphenylcyclotetrasiloxane), or a combination thereof.

The dopant material may include a phosphorescent dopant, a fluorescent dopant, or a combination thereof. For example, the dopant material may include a metal complex containing iridium (Ir), platinum (Pt), osmium (Os), gold (Au), titanium (Ti), zirconium (Zr), hafnium (Hf), europium (Eu), terbium (Tb), or thulium (Tm), or BCzVB (1,4-bis[2-(3-N-ethylcarbazoryl)vinyl]benzene), DPAVB (4 (di-p-tolylamino)-4′-[(di-p-tolylamino)styryl]stilbene), N-BDAVBi (N-(4-((E)-2-(6-((E)-4 (diphenylamino) styryl) naphthalen-2-yl) vinyl)phenyl)-N-phenylbenzenamine), DPAVBi (4,4′-bis[2-(4 (N,N-diphenylamino)phenyl) vinyl]biphenyl), TBP (2,5,8,11-tetra-t-butylperylene), or a combination thereof.

In one embodiment, the host material may include a heterocyclic compound represented by Formula 1 above. For example, the heterocyclic compound may serve as a red phosphorescent dopant.

By including the heterocyclic compound as the host material, the charge transport properties within the light-emitting device and the stability of the light-emitting layer 303 may be further improved, thereby increasing the light-emitting efficiency and lifetime of the light-emitting device. Accordingly, the light-emitting properties may be improved without increasing the operating voltage.

In some embodiments, the content of the dopant material in the light-emitting layer 303 may be about 0.01 parts by weight or more, about 1 part by weight or more, or about 2 parts by weight or more, and may be about 15 parts by weight or less, about 10 parts by weight or less, or about 8 parts by weight or less, based on 100 parts by weight of the host material. Within this range, the formation and emission energy of excitons may be increased, and the light-emitting efficiency and stability may be further improved.

In some embodiments, the organic layer 300 may further include a compound represented by Formula 3 below.

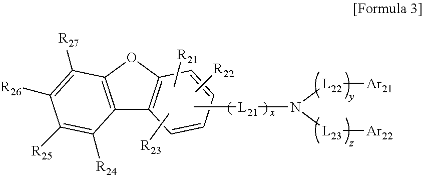

In Formula 3, two adjacent groups of R₂₁ to R₂₇ are linked to each other to form an aromatic ring represented by

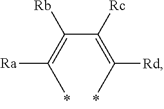

any one of the remainders of R₂₁ to R₂₇ that does not form a ring is a substituted or unsubstituted aryl group having 6 to 60 carbon atoms, or a substituted or unsubstituted heteroaryl group having 2 to 60 carbon atoms, and

Ra, Rb, Rc, Rd, and the remaining substituents are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(—O)RR′, or adjacent groups are linked to form a substituted or unsubstituted 5- to 9-membered saturated or unsaturated ring,

L₂₁ to L₂₃ are each independently a direct linkage, a substituted or unsubstituted arylene group having 6 to 60 carbon atoms, or a substituted or unsubstituted heteroarylene group having 2 to 60 carbon atoms,
x, y, and z are each independently 0 or an integer from 1 to 5,
when x is an integer from 2 to 5, a plurality of L₂₁ groups are the same or different,
when y is an integer from 2 to 5, a plurality of L₂₂ groups are the same or different,
when z is an integer from 2 to 5, a plurality of L₂₃ groups are the same or different, and
Ar₂₁ and Ar₂₂ are each independently a substituted or unsubstituted aryl group having 6 to 60 carbon atoms, or a substituted or unsubstituted heteroaryl group having 2 to 60 carbon atoms.

According to exemplary embodiments, in Formula 3, two adjacent groups of R₂₁ to R₂₇ may be linked to each other to form an aromatic ring represented by

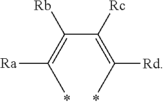

For example, R₂₄ and R₂₅ may be linked to each other to form an aromatic ring represented by

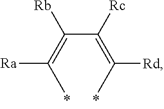

or R₂₅ and R₂₆ may be linked to each other to form an aromatic ring represented by

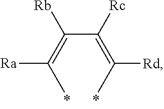

or R₂₆ and R₂₇ may be linked to each other to form an aromatic ring represented by

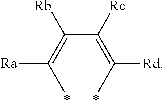

According to exemplary embodiments, the compound represented by Formula 3 may be represented by any one of Formulae 3-1 to 3-6 below.

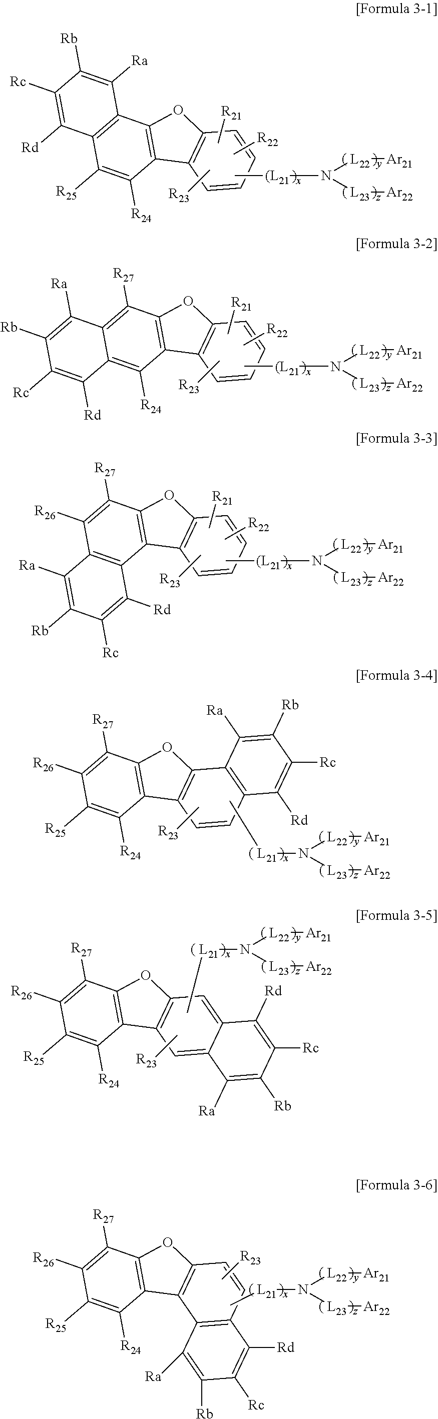

In Formulae 3-1 to 3-6, R₂₁ to R₂₇, Ra, Rb, Rc, Rd, L₂₁ to L₂₃, Ar₂₁, Ar₂₂, x, y, and z may be the same as defined above for Formula 3.

According to exemplary embodiments, in Formulae 3 and Formulae 3-1 to 3-6, L₂₁, L₂₂ and L₂₃ may each independently be a direct linkage, a substituted or unsubstituted arylene group having 6 to 40 carbon atoms, or a substituted or unsubstituted heteroarylene group having 2 to 40 carbon atoms. According to some embodiments, L₂₁, L₂₂ and L₂₃ may each independently be a direct linkage, a substituted or unsubstituted arylene group having 6 to 20 carbon atoms, or a substituted or unsubstituted heteroarylene group having 2 to 15 carbon atoms. L₂₁, L₂₂ and L₂₃ may be the same or different.

For example, L₂₁, L₂₂ and L₂₃ may each independently be a direct linkage, substituted, or unsubstituted arylene group having 6 to 20 carbon atoms.

For example, L₂₁, L₂₂ and L₂₃ may each independently be a direct linkage, substituted, or unsubstituted phenylene group, or a substituted, or unsubstituted naphthalene group.

For example, L₂₁, L₂₂ and L₂₃ may each independently be a direct linkage, substituted, or unsubstituted arylene group having 6 to 20 carbon atoms, or a deuterium-substituted, or unsubstituted phenylene group.

For example, L₂₁, L₂₂ and L₂₃ may each independently be a direct linkage, substituted, or unsubstituted phenylene group, or substituted, or unsubstituted phenylene group.

According to exemplary embodiments, in Formula 3 and Formulae 3-1 to 3-6, x, y, and z may each independently be 0 or an integer from 1 to 5. For example, x, y, and z may be 0, 1, or 2.

When x is an integer from 2 to 5, a plurality of L₂₁ groups may be the same or different. When y is an integer from 2 to 5, a plurality of L₂₂ groups may be the same or different. When z is an integer from 2 to 5, a plurality of L₂₃ groups may be the same or different.

According to exemplary embodiments, in Formula 3 and Formulae 3-1 to 3-6, Ar₂₁ and Ar₂₂ may each independently be a substituted or unsubstituted aryl group having 6 to 60 carbon atoms, or a substituted or unsubstituted heteroaryl group having 2 to 60 carbon atoms.

In some embodiments, Ar₂₁ and Ar₂₂ may each independently be a substituted or unsubstituted aryl group having 6 to 40 carbon atoms, or a substituted or unsubstituted heteroaryl group having 2 to 40 carbon atoms.

In some embodiments, Ar₂₁ and Ar₂₂ may each independently be a substituted or unsubstituted aryl group having 6 to 20 carbon atoms, or a substituted or unsubstituted heteroaryl group having 2 to 15 carbon atoms.

For example, Ar₂₁ and Ar₂₂ may each independently be a substituted or unsubstituted phenyl group; a substituted or unsubstituted biphenyl group; a substituted or unsubstituted terphenyl group; a substituted or unsubstituted naphthyl group; a substituted or unsubstituted phenanthryl group; a substituted or unsubstituted triphenylenyl group; a substituted or unsubstituted anthracenyl group; a substituted or unsubstituted dimethylfluorenyl group; a substituted or unsubstituted diethylfluorenyl group; a substituted or unsubstituted spirobifluorenyl group; a substituted or unsubstituted furan group; a substituted or unsubstituted thiophenyl group; a substituted or unsubstituted benzofuran group; a substituted or unsubstituted benzothiophene group; a substituted or unsubstituted dibenzofuran group; a substituted or unsubstituted dibenzothiophene group; a substituted or unsubstituted carbazolyl group; or a combination thereof.

According to exemplary embodiments, in Formula 3 and Formulae 3-1 to 3-6, Ar₂₁ and Ar₂₂ may each independently be a substituted or unsubstituted aryl group having 6 to 60 carbon atoms, or a substituted or unsubstituted heteroaryl group having 2 to 60 carbon atoms, each of which may be substituted or unsubstituted with deuterium.

According to some embodiments, Ar₂₁ and Ar₂₂ may each independently be a substituted or unsubstituted aryl group having 6 to 40 carbon atoms, or a substituted or unsubstituted heteroaryl group having 2 to 40 carbon atoms, each of which may be substituted or unsubstituted with deuterium.

According to some embodiments, Ar₂₁ and Ar₂₂ may each independently be a substituted or unsubstituted aryl group having 6 to 20 carbon atoms, or a substituted or unsubstituted heteroaryl group having 2 to 15 carbon atoms.

For example, Ar₂₁ and Ar₂₂ may each independently be a phenyl group substituted or unsubstituted with deuterium; a biphenyl group substituted or unsubstituted with deuterium; a terphenyl group substituted or unsubstituted with deuterium; a naphthyl group substituted or unsubstituted with deuterium; a phenanthryl group substituted or unsubstituted with deuterium; a triphenylenyl group substituted or unsubstituted with deuterium; a dimethylfluorenyl group substituted or unsubstituted with deuterium; a diphenylfluorenyl group substituted or unsubstituted with deuterium; a spirobifluorenyl group substituted or unsubstituted with deuterium; a furan group substituted or unsubstituted with deuterium; a thiophene group substituted or unsubstituted with deuterium; a benzofuran group substituted or unsubstituted with deuterium; a benzothiophene group substituted or unsubstituted with deuterium; a dibenzofuran group substituted or unsubstituted with deuterium; a dibenzothiophene group substituted or unsubstituted with deuterium; an anthracenyl group substituted or unsubstituted with deuterium; a carbazolyl group substituted or unsubstituted with deuterium; or a combination thereof.

According to exemplary embodiments, the deuterium content of the compound of Formula 3 may be 0%, or greater than 0 and not more than 100%.

According to exemplary embodiments, the deuterium content of the compound of Formula 3 may be 0%, or 5% to 100%.

According to exemplary embodiments, the deuterium content of the compound of Formula 3 may be 0%, or 10% to 100%.

According to exemplary embodiments, the deuterium content of the compound of Formula 3 may be 0%, or 15% to 100%.

According to exemplary embodiments, the deuterium content of the compound of Formula 3 may be 0%, or 20% to 100%.

According to exemplary embodiments, the deuterium content of the compound of Formula 3 may be 0%, or 25% to 100%.

According to exemplary embodiments, the deuterium content of the compound of Formula 3 may be 0%, or 30% to 100%.

According to exemplary embodiments, the deuterium content of the compound of Formula 3 may be 0%, or 50% to 100%.

According to exemplary embodiments, the deuterium content of the compound of Formula 3 may be 0%, or 70% to 100%.

According to exemplary embodiments, the deuterium content of the compound of Formula 3 may be 0%, or 90% to 100%.

According to exemplary embodiments, the deuterium content of the compound of Formula 3 may be 0%.

According to exemplary embodiments, the deuterium content of the compound of Formula 3 may be 30% to 100%.

According to exemplary embodiments, the deuterium content of the compound of Formula 3 may be 50% to 100%.

According to exemplary embodiments, the deuterium content of the compound of Formula 3 may be 70% to 100%.

According to exemplary embodiments, the deuterium content of the compound of Formula 3 may be 90% to 100%.

In some embodiments, the compound represented by Formula 3 may be represented by at least one selected from formulae below:

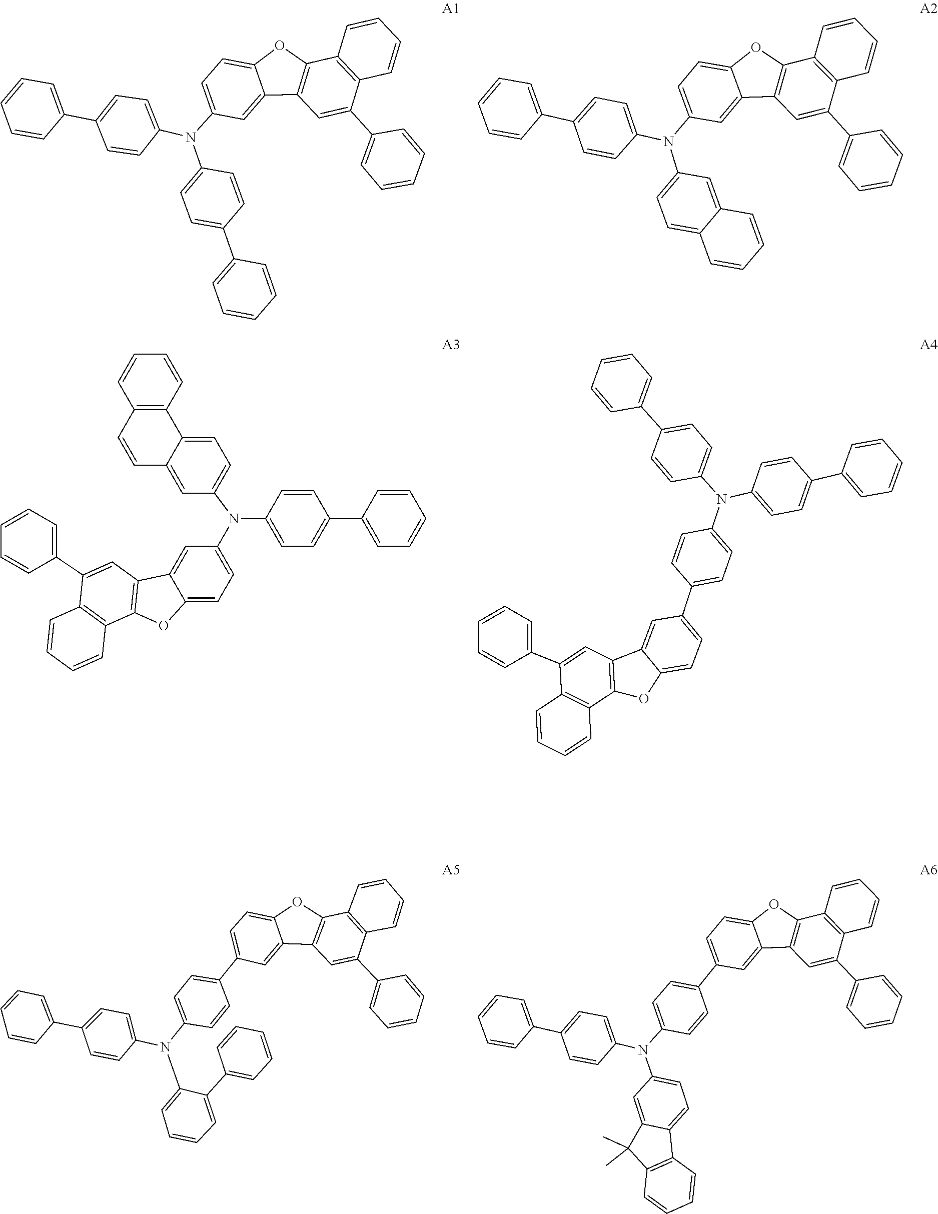

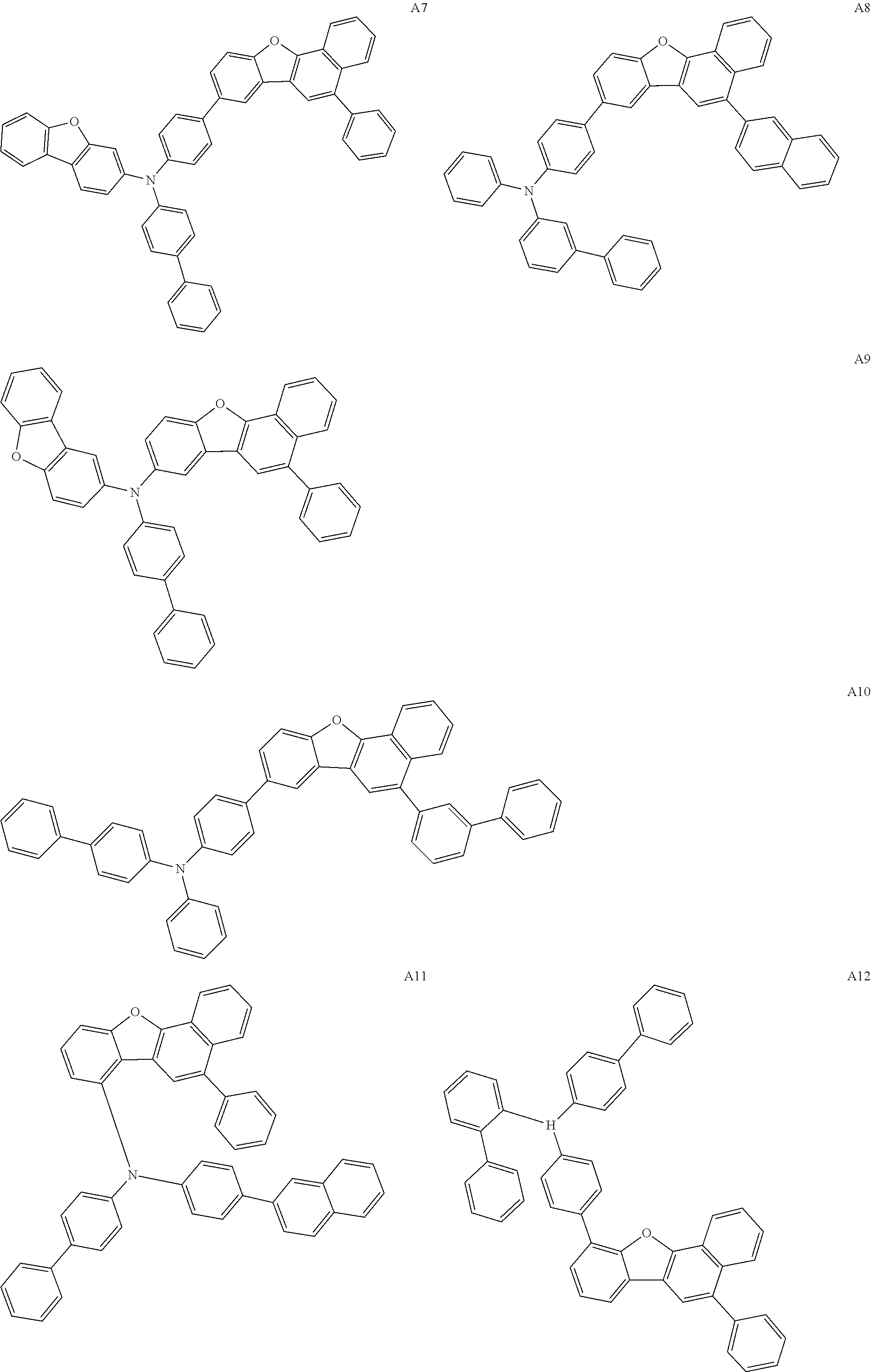

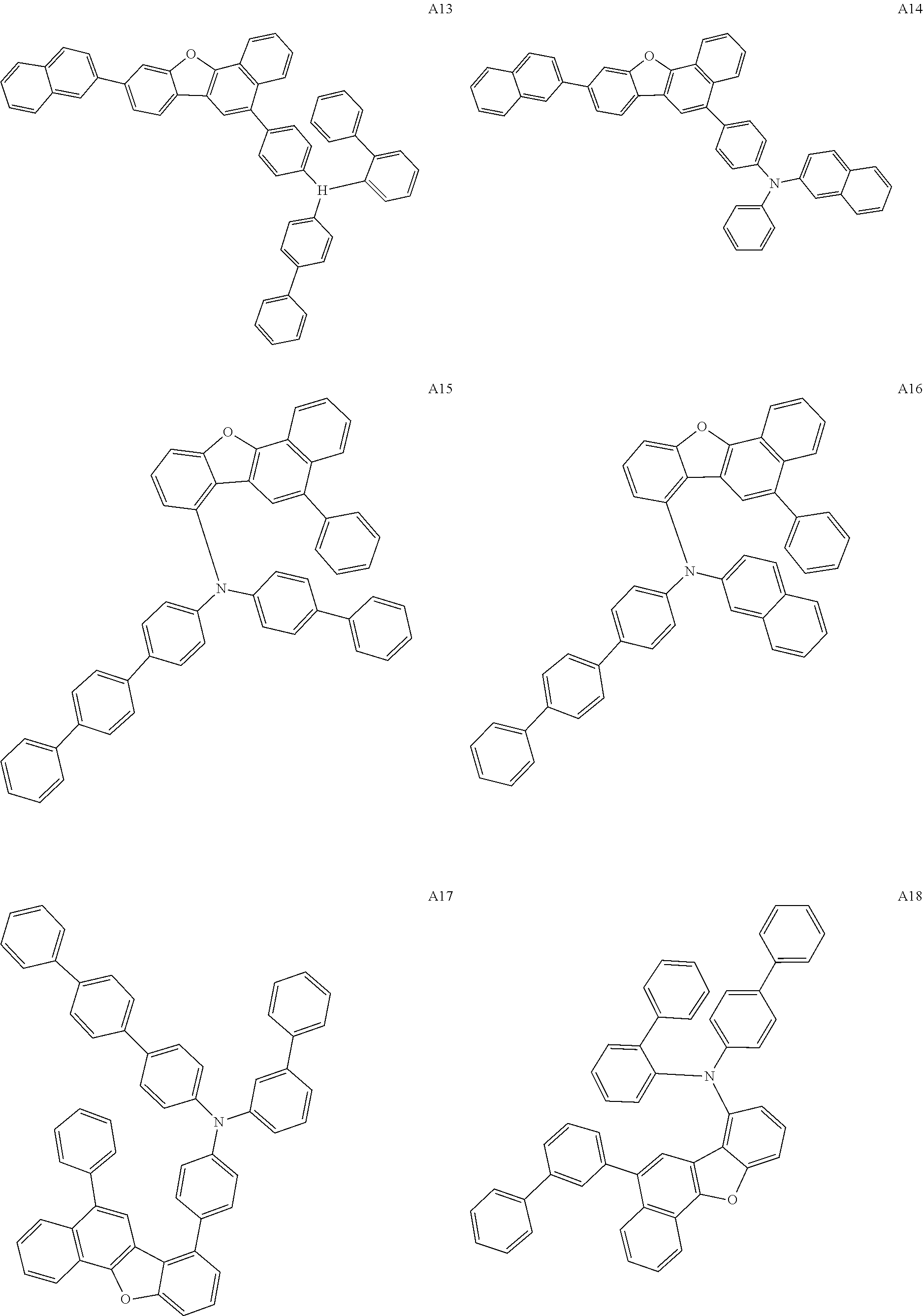

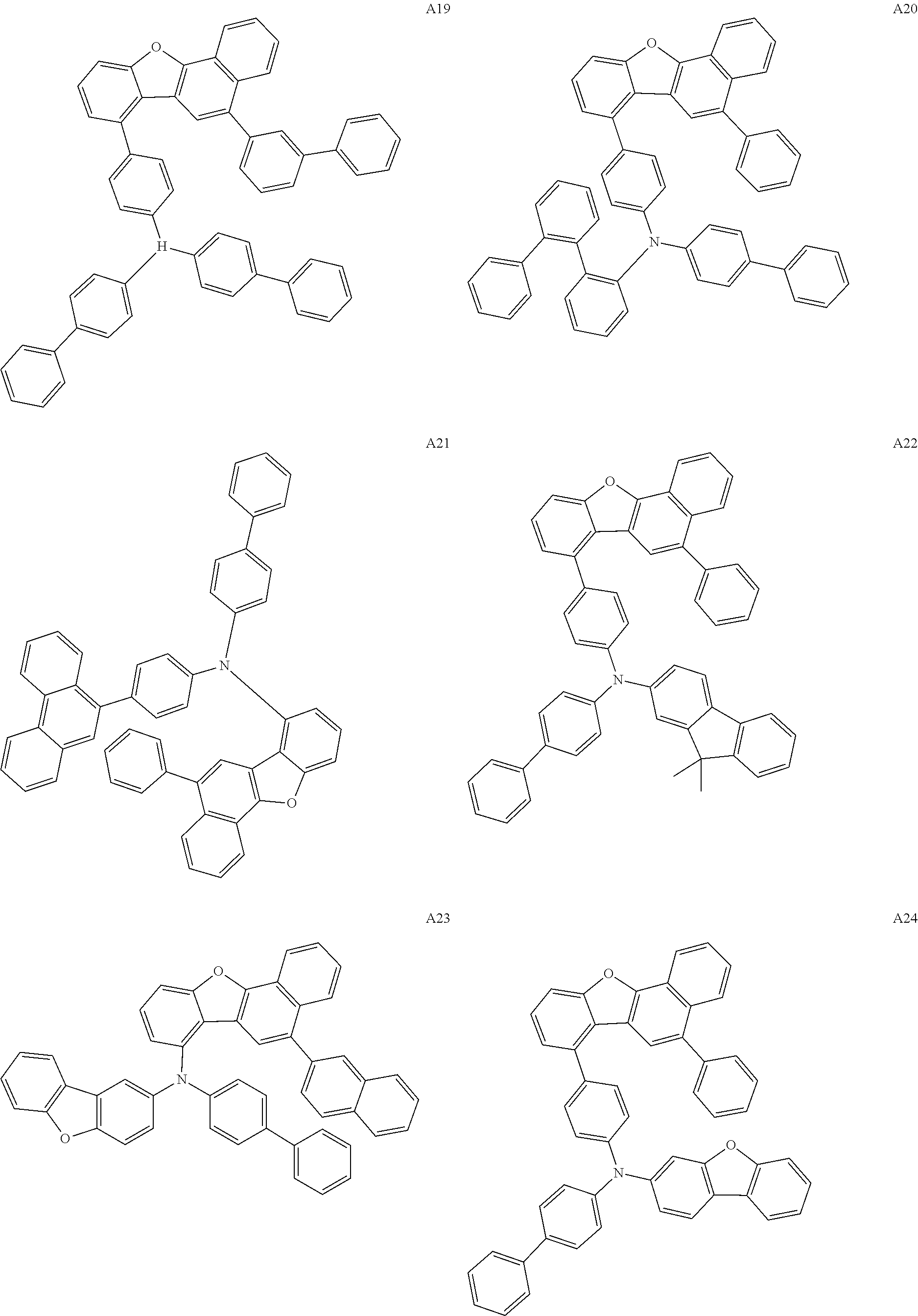

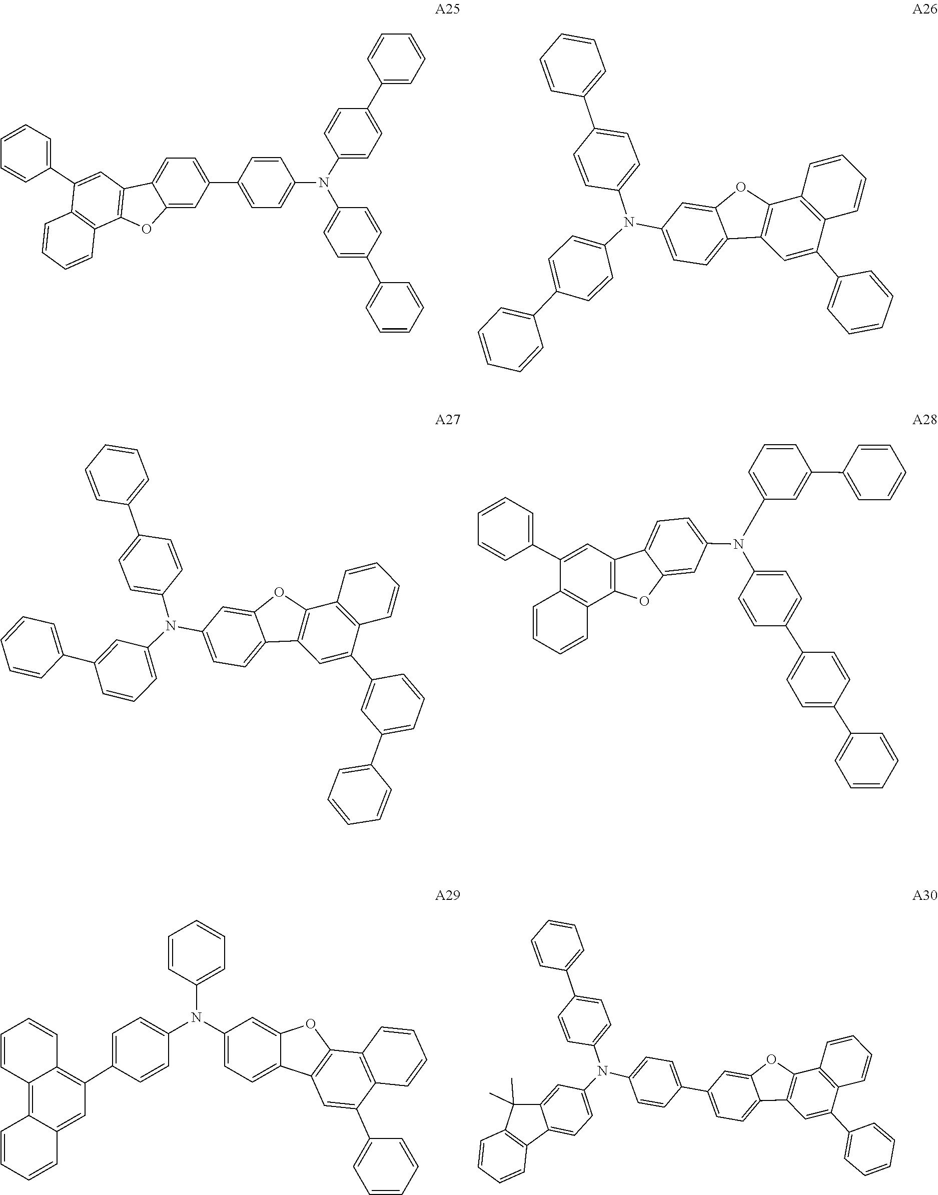

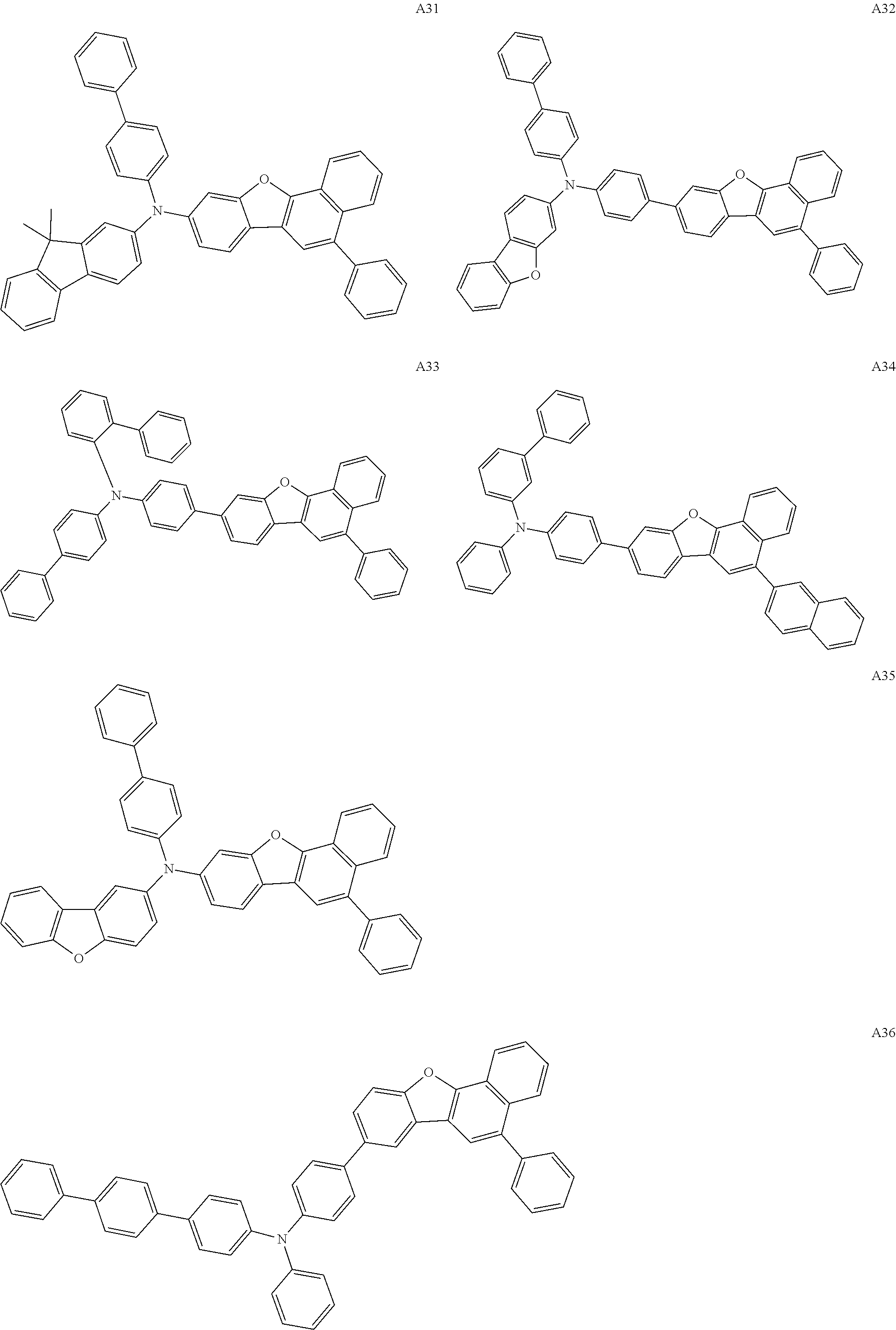

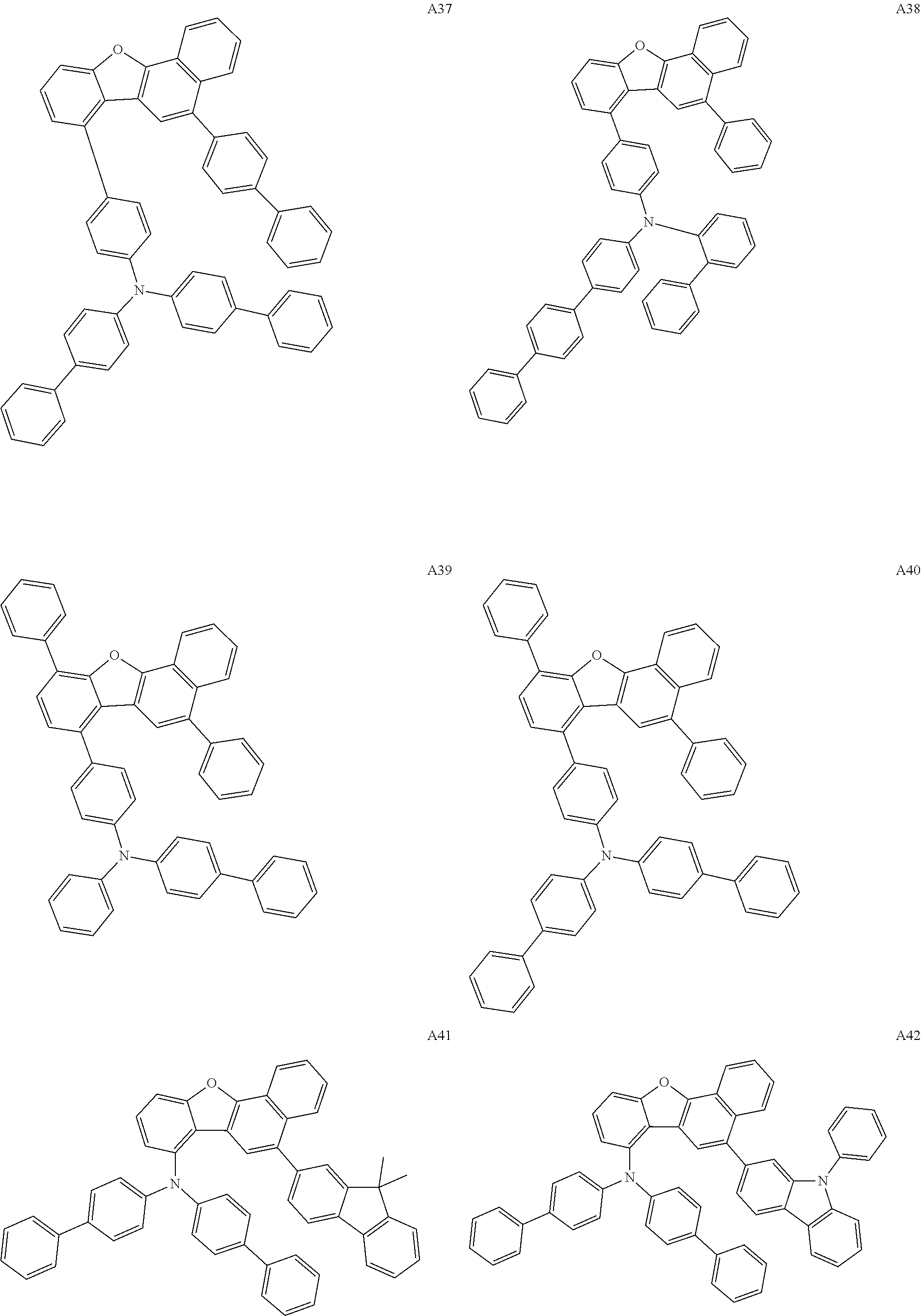

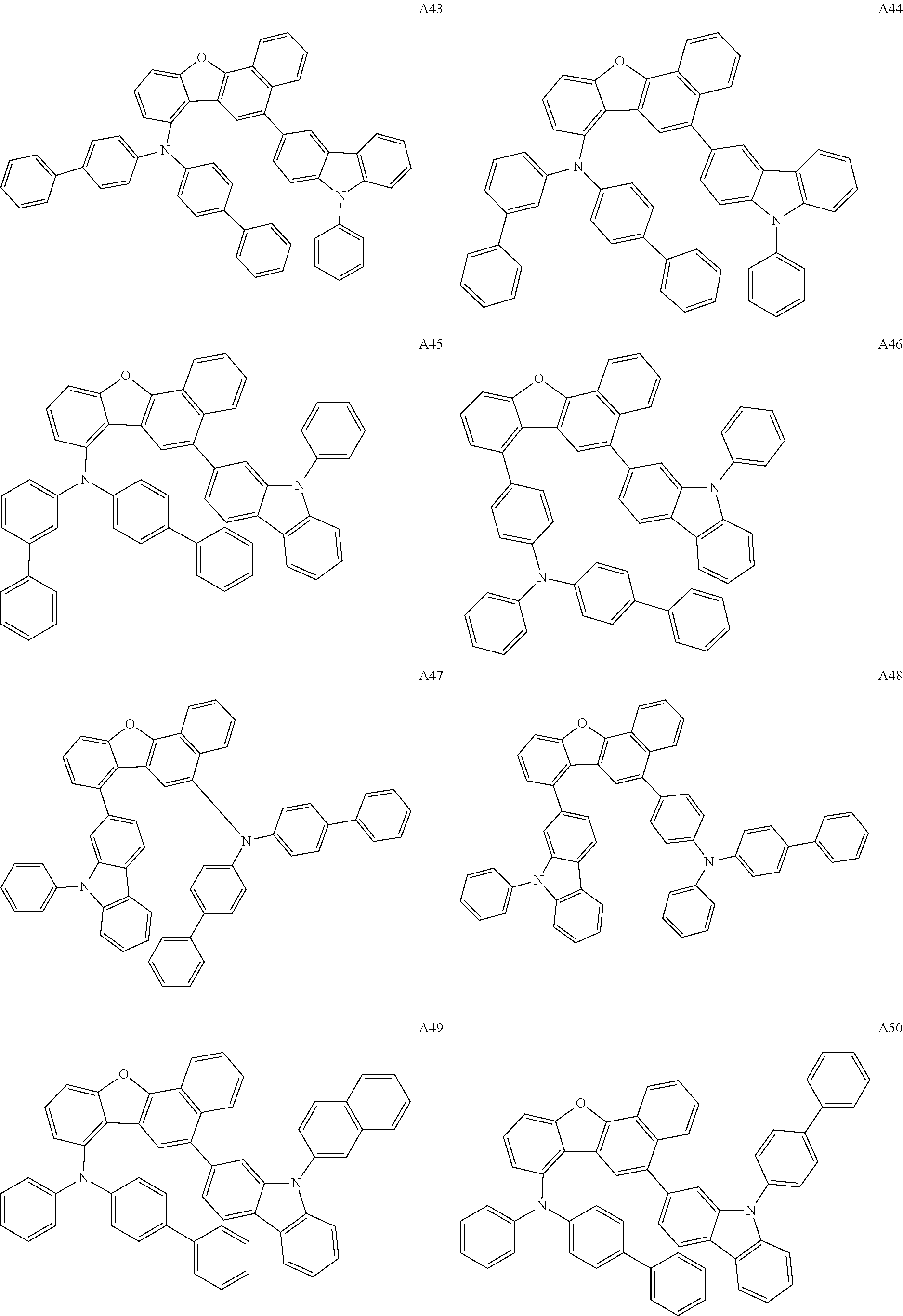

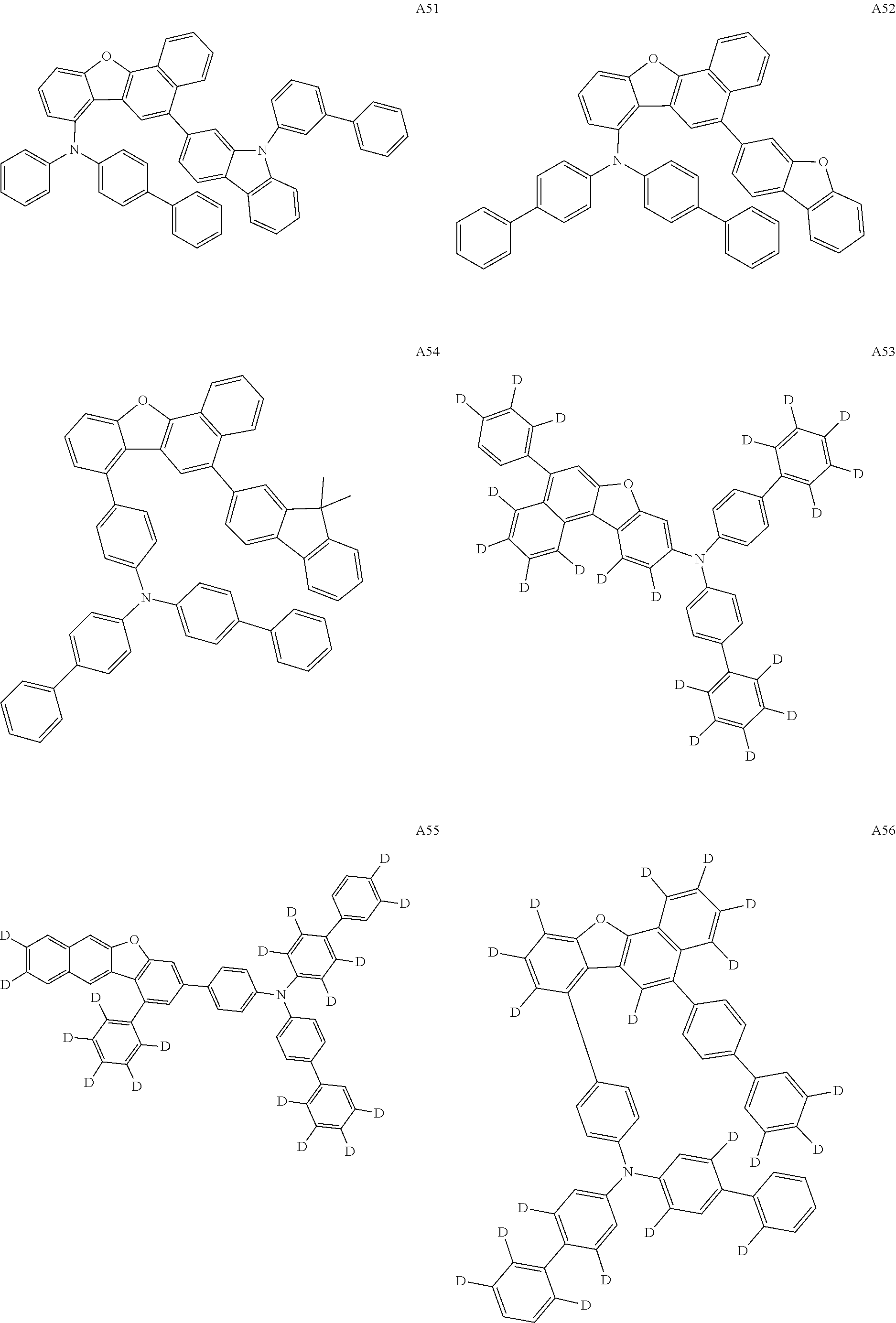

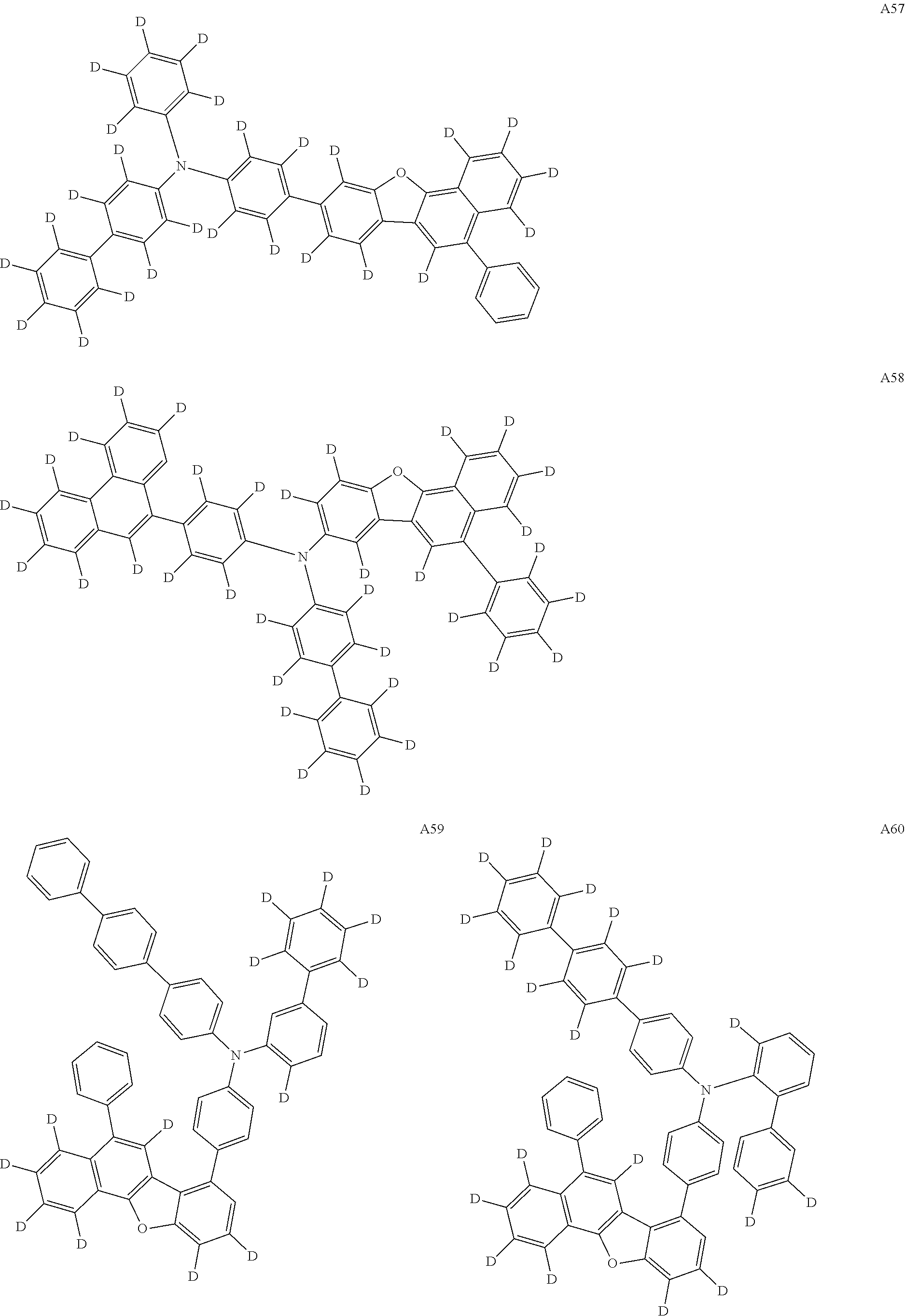

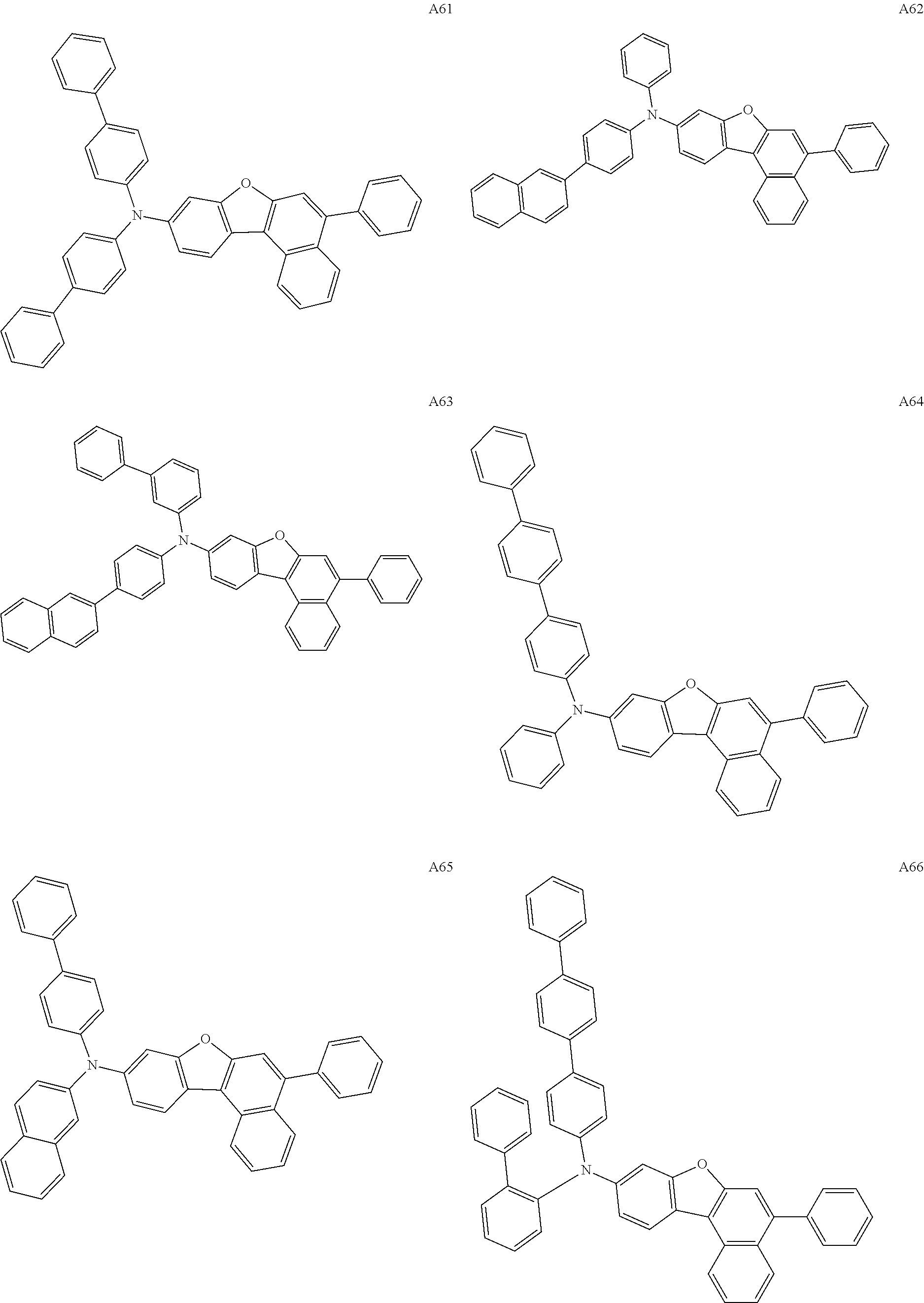

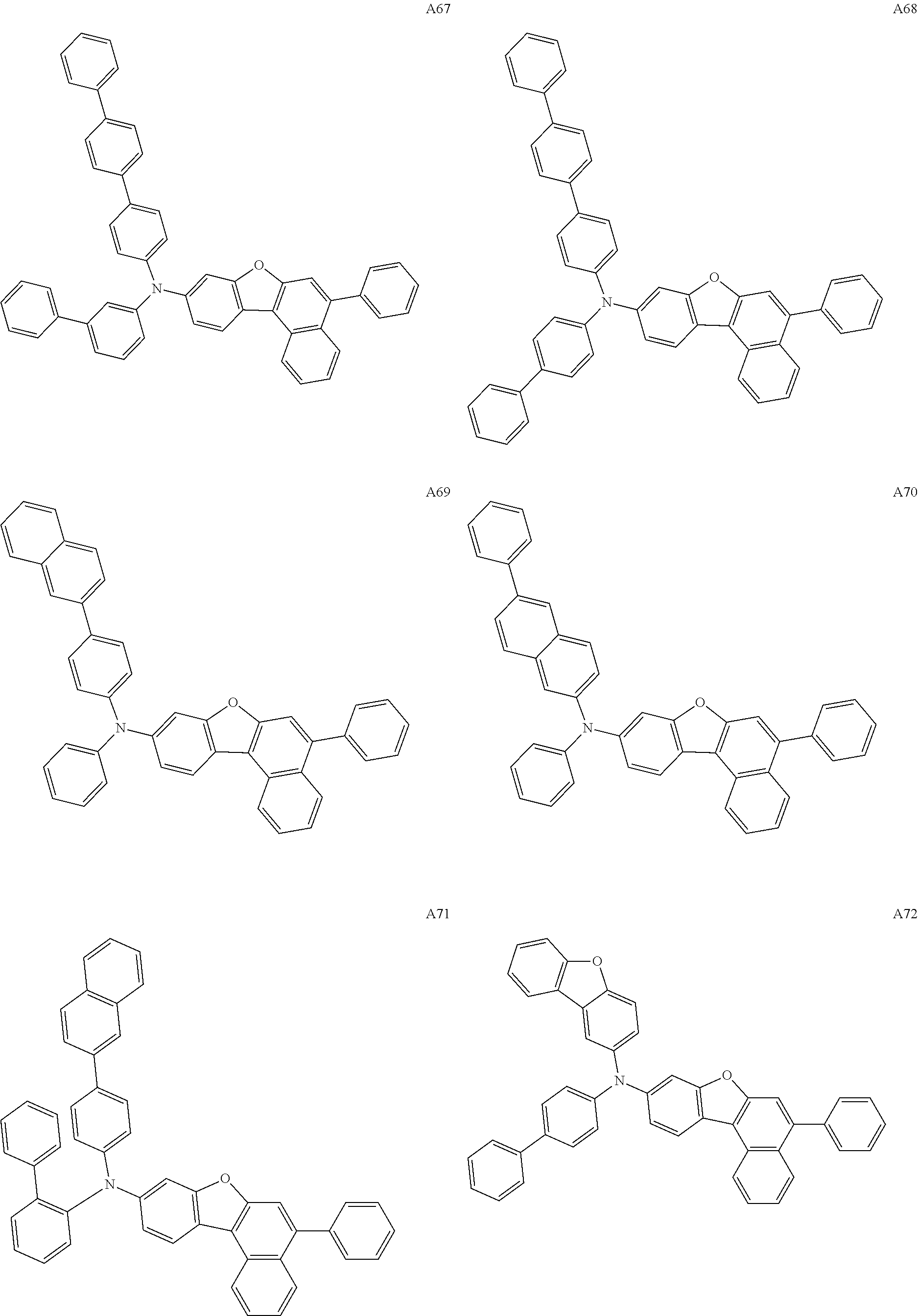

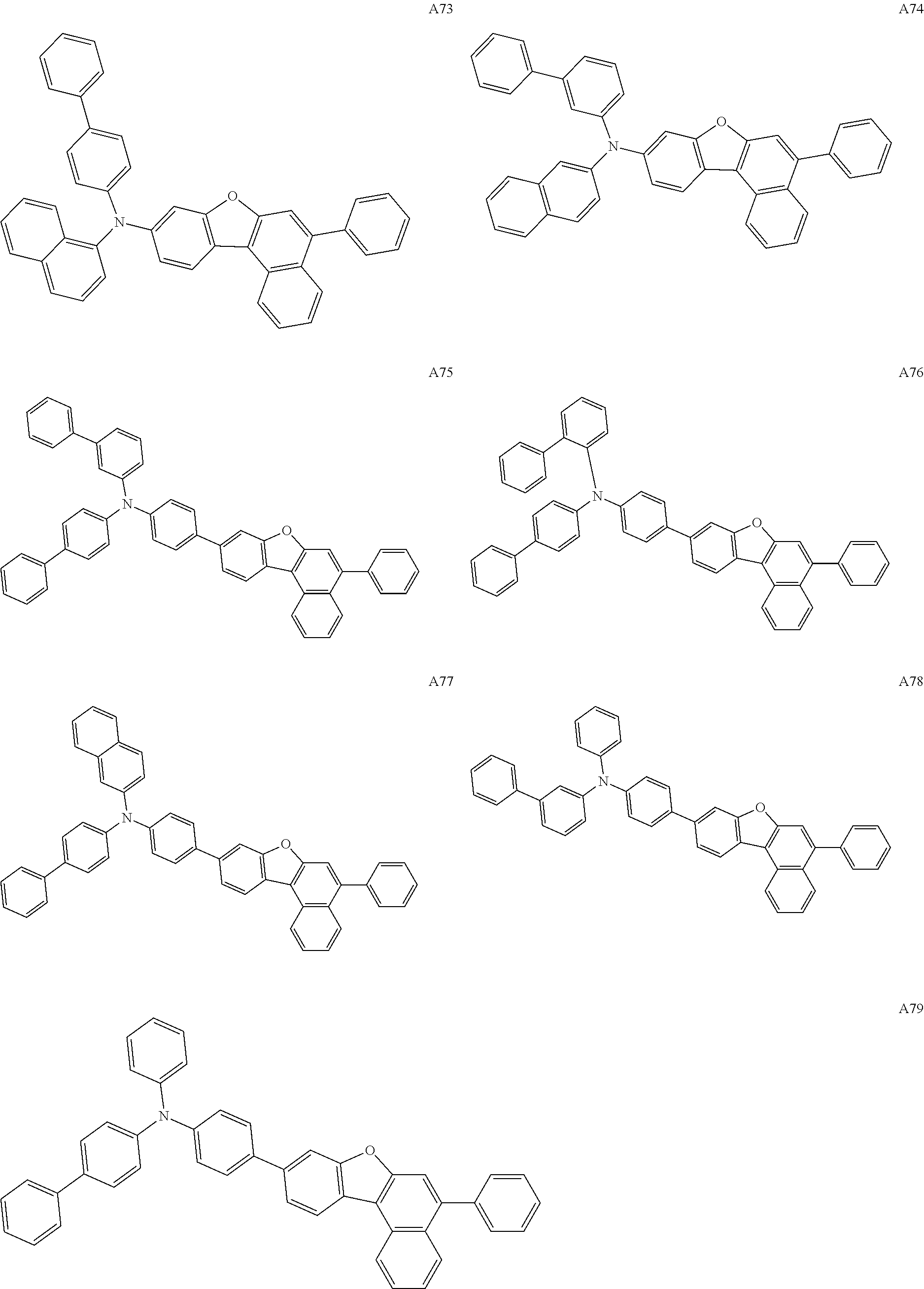

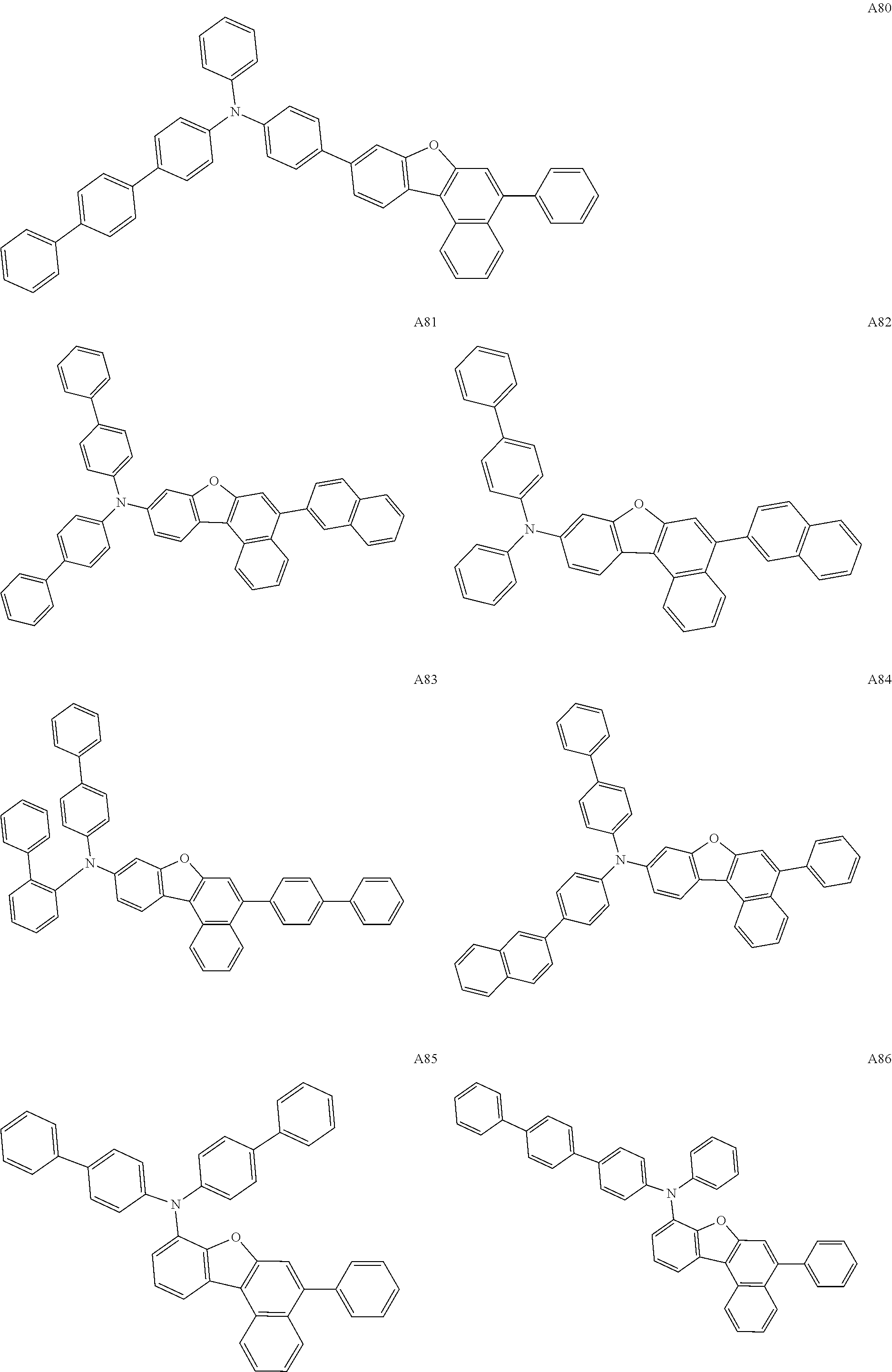

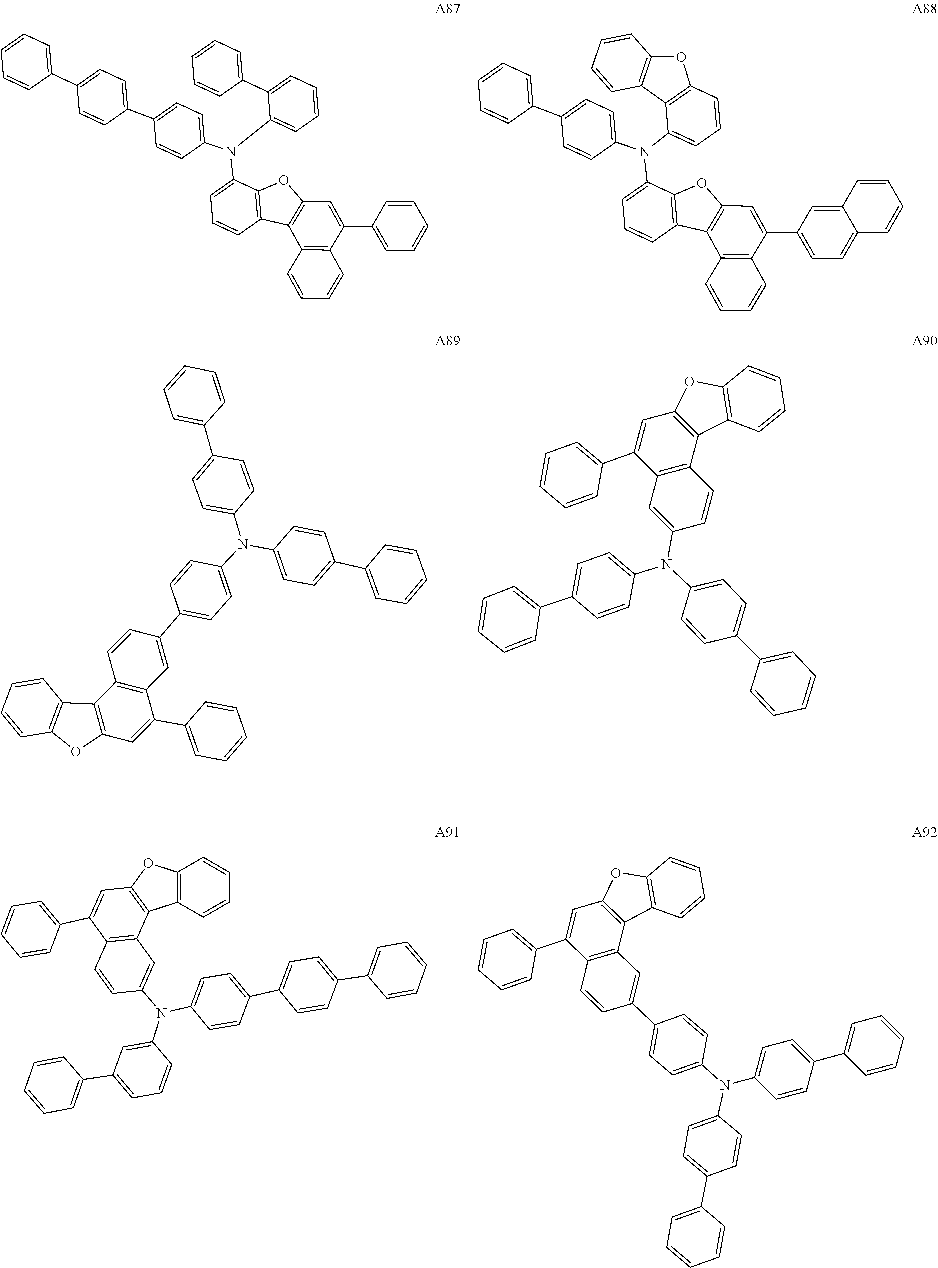

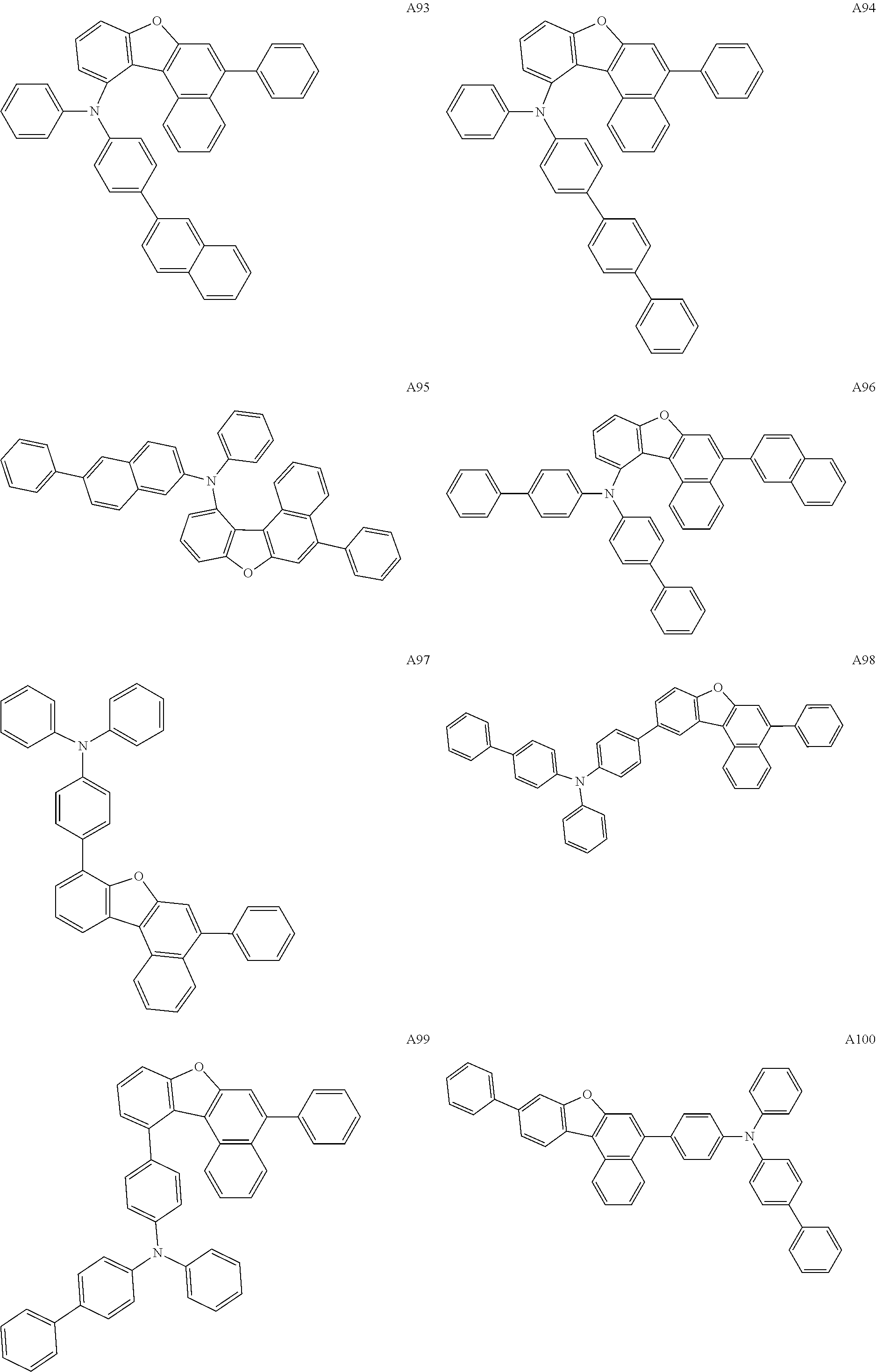

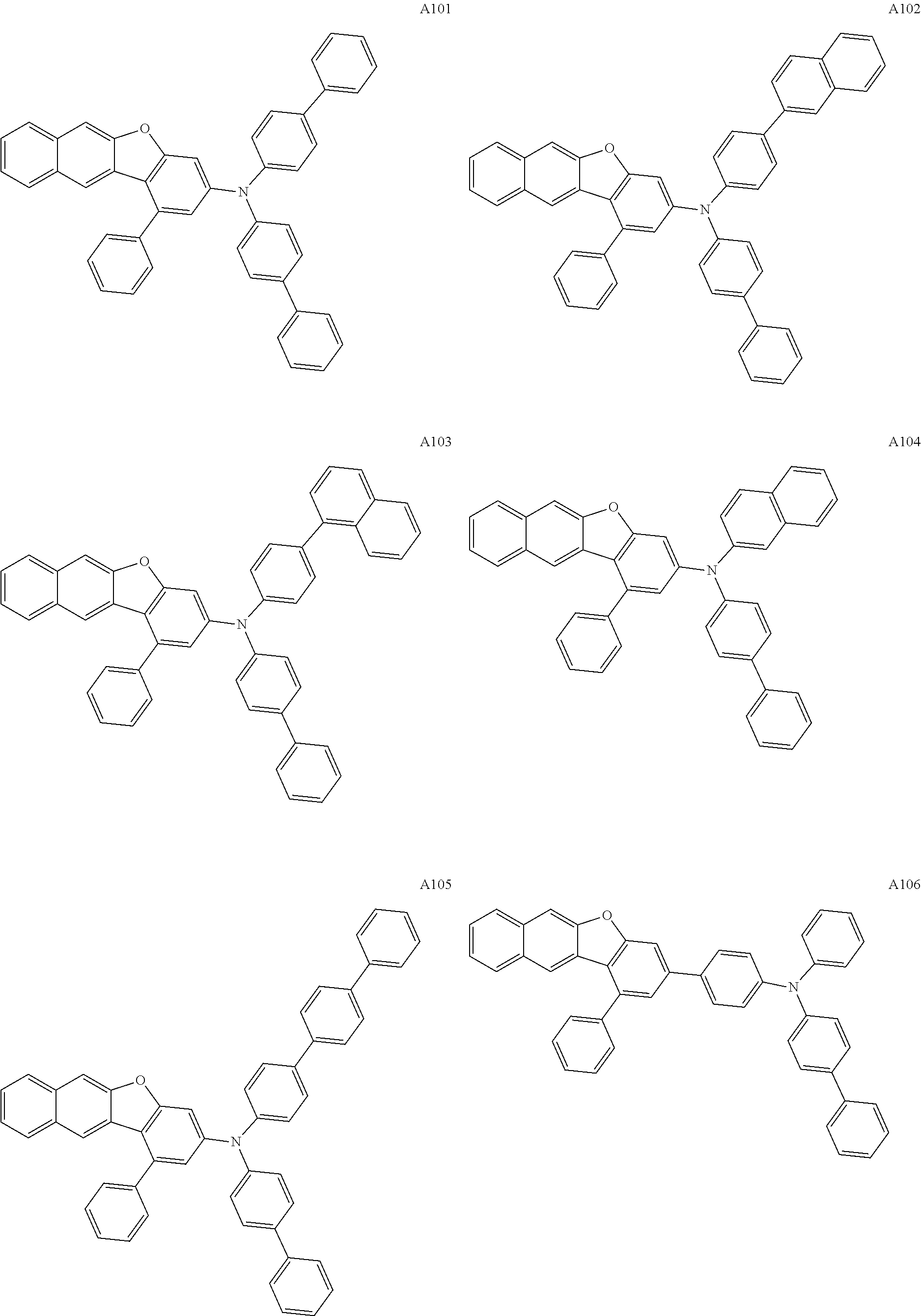

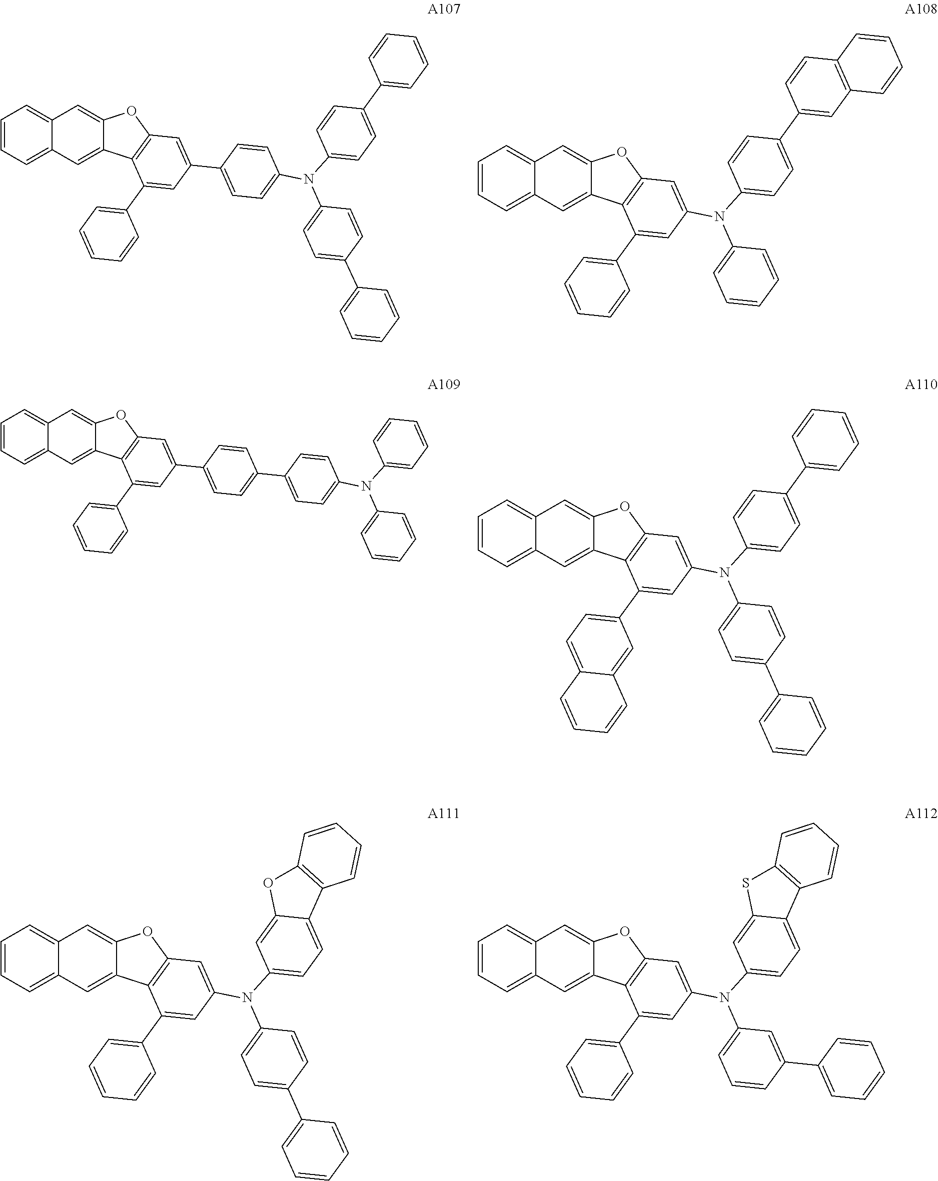

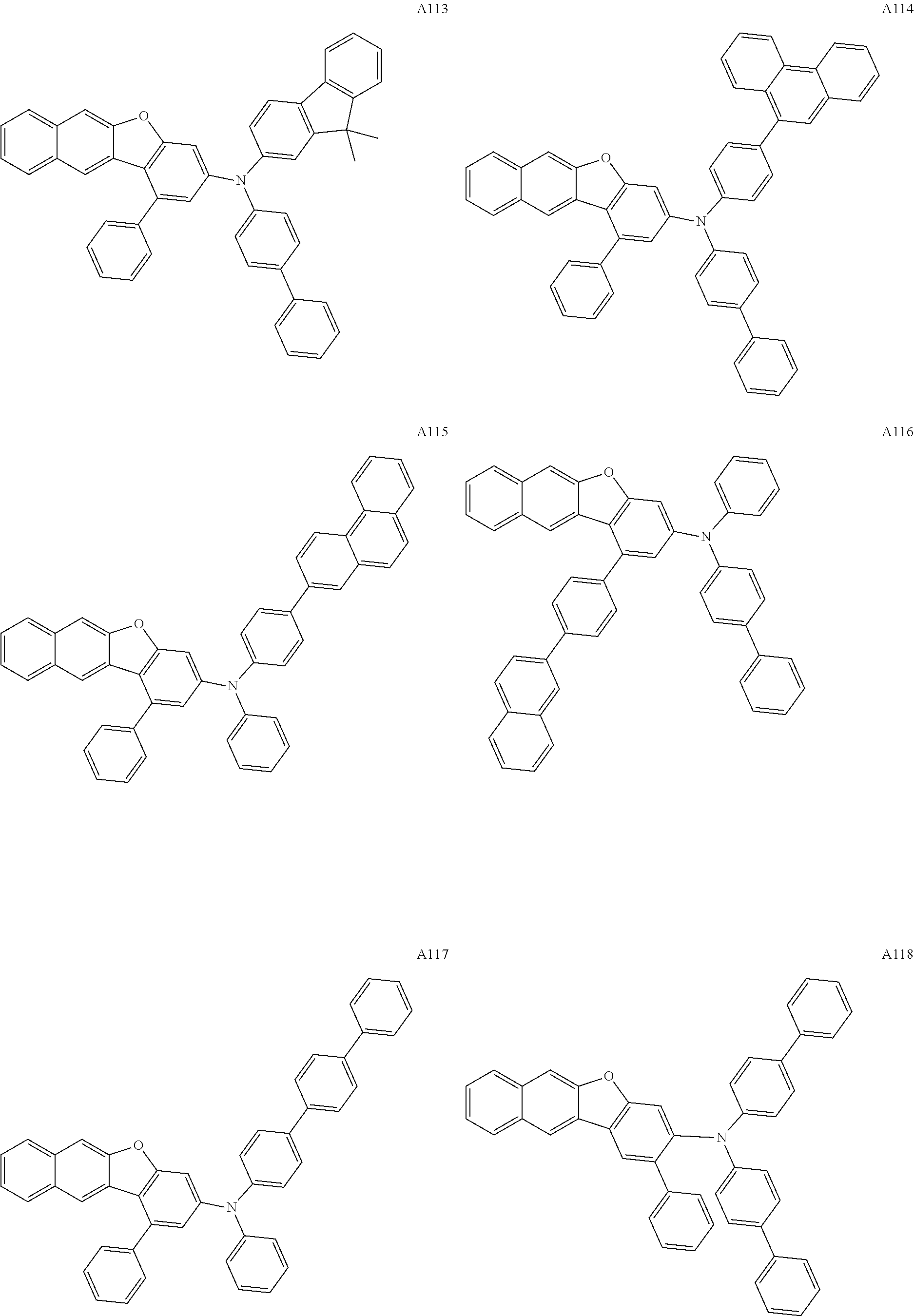

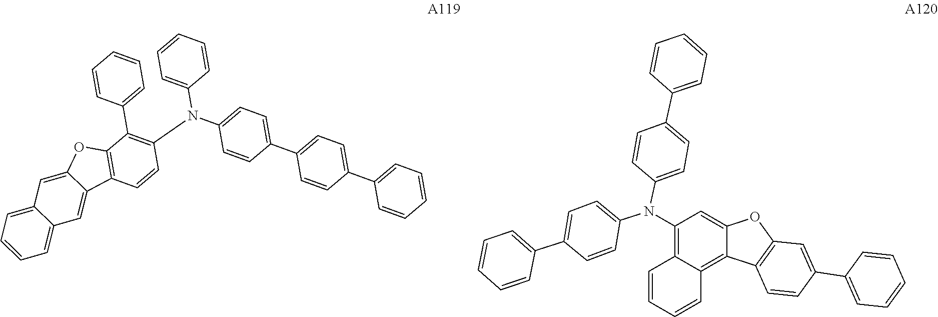 [SMILES: c1ccc(-c2ccc(-c3ccc(N(c4ccccc4)c4ccc5c(oc6cc7ccccc7cc65)c4-c4ccccc4)cc3)cc2)cc1 ; c1ccc(-c2ccc(N(c3ccc(-c4ccccc4)cc3)c3cc4oc5cc(-c6ccccc6)ccc5c4c4ccccc34)cc2)cc1]

When the organic layer 300 includes the compound represented by Formula 3 together with the heterocyclic compound, the electron and hole transport characteristics of the light-emitting device may be further enhanced, and the light-emitting efficiency and device lifetime may be further improved.

In one embodiment, the compound represented by Formula 3 may be included in the same organic layer as the heterocyclic compound, and for example, may be included in the light-emitting layer 303. For example, the heterocyclic compound represented by Formula 1 may be an n-type host material, and the compound represented by Formula 3 may be a p-type host material.

For example, the heterocyclic compound represented by Formula 1 may have a high electron transport ability, thereby acting as a donor, and the compound represented by Formula 3 may have a high hole transport ability, thereby acting as an acceptor. When these compounds are used together, an exciplex phenomenon may occur, thereby improving charge balance within the device.

In one embodiment, the weight ratio of the heterocyclic compound to the compound represented by Formula 3 in the organic layer 300 or the light-emitting layer 303 may be 1:10 to 10:1, 1:8 to 8:1, 1:5 to 5:1, or 1:2 to 2:1. Within this range, both electron transport characteristics and hole transport characteristics in the organic layer 300 may be improved. Accordingly, the operating voltage of the light-emitting device may be decreased, and the light-emitting efficiency and lifetime characteristics may be further improved.

As the material of the hole injection layer 301, known hole injection layer materials may be used. For example, phthalocyanine compounds such as copper phthalocyanine disclosed in U.S. Pat. No. 4,356,429, or a starburst amine derivative described in the literature[Advanced Materials, 6, p. 677 (1994)], such as tris(4-carbazoyl-9-ylphenyl)amine (TCTA), 4,4′, 4″-tri[phenyl(m-tolyl)amino]triphenylamine (m-MTDATA), 1,3,5-tris[4-(3-methylphenylphenylamino)phenyl]benzene (m-MTDAPB), 4,4′, 4″-tris(N,N-(2-naphthyl)-phenylamino)triphenylamine (2-TNATA), or a soluble conductive polymer such as polyaniline/dodecylbenzenesulfonic acid poly(3,4-ethylenedioxythiophene)/poly(4-styrenesulfonate), polyaniline/camphorsulfonic acid, or polyaniline/poly(4-styrenesulfonate) may be used.

As the materials of the hole transport layer 302, pyrazoline derivatives, arylamine derivatives, stilbene derivatives, or triphenyldiamine derivatives may be used and Low-molecular-weight or high-molecular-weight materials may also be used. As the materials of the electron transport layer, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, or 8-hydroxyquinoline and metal complexes thereof may be used. Not only low-molecular-weight materials but also high-molecular-weight materials may be used.

For example, LiF is typically used in the art as the material of the electron injection layer 305, but the present application is not limited thereto.

As the materials of the electron transport layer 304, anthracene compounds, Alq3 (tris(8-hydroxyquinolinato)aluminum), 1,3,5-tri[(3-pyridyl)-phen-3-yl]benzene, 2,4,6-tris(3′-(pyridin-3-yl) biphenyl-3-yl)-1,3,5-triazine, 2-(4 (N-phenylbenzoimidazol-1-yl)phenyl)-9,10-dinaphthylanthracene, TPBi (1,3,5-tri (1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4 (naphthalen-1-yl)-3,5-diphenyl-4H-1,2,4-triazole), tBu-PBD (2-(4-biphenylyl)-5-(4-tert-butylphenyl)-1,3,4 oxadiazole), BAlq (bis(2-methyl-8-quinolinolato-N1,O8)-(1,1′-biphenyl-4-olato)aluminum), Bebq2 (beryllium bis(benzoquinolin-10-olate)), ADN (9,10-di(naphthalene-2-yl) anthracene), BmPyPhB (1,3-bis[3,5-di(pyridin-3-yl)phenyl]benzene), or a combination thereof may be used.

According to an exemplary embodiment, the light-emitting device may include a first electrode, a second electrode, and two or more stacks provided between the first electrode and the second electrode. Each of the two or more stacks may independently include a light-emitting layer, and a charge generation layer may be provided between the two or more stacks.

FIG. 4 is a schematic cross-sectional view illustrating a light-emitting device having a two-stack tandem structure according to an exemplary embodiment.

Referring to FIG. 4, the light-emitting device may include a first electrode, a first stack disposed on the first electrode and including a first stack light-emitting layer, a charge generation layer disposed on the first stack, a second stack disposed on the charge generation layer and including a second stack light-emitting layer, and a second electrode disposed on the second stack. At least one of the first stack and the second stack may include the heterocyclic compound represented by Formula 1.

In addition, the first stack and the second stack may each independently further include one or more of the above-described hole injection layer, hole transport layer, hole blocking layer, electron transport layer, and electron injection layer.

According to an exemplary embodiment, the first stack may include a heterocyclic compound represented by Formula 1.

According to an exemplary embodiment, the second stack may include a heterocyclic compound represented by Formula 1.

According to an exemplary embodiment, the first stack light-emitting layer of the first stack may include a heterocyclic compound represented by Formula 1.

According to an exemplary embodiment, the second stack light-emitting layer of the second stack may include a compound represented by Formula 1.

As used herein, the charge generation layer may be an N-type charge generation layer, and may further include a dopant known in the art.

According to exemplary embodiments, a method for manufacturing the light-emitting device may be provided.

According to exemplary embodiments, a substrate may be prepared. A first electrode may be formed on the substrate. One or more organic layers may be formed on the first electrode. A second electrode may be formed on the organic layer to manufacture a light-emitting device.

The light-emitting device may be manufactured using conventional methods and materials for manufacturing a light-emitting device, except that one or more organic layers are formed using the above-described heterocyclic compound.

When manufacturing an organic light-emitting device, an organic layer including the heterocyclic compound may be formed using a vacuum deposition method. Alternatively, the organic layer may be formed by applying a composition for an organic layer including the heterocyclic compound using a solution application method. Here, the solution application method may include spin coating, dip coating, inkjet printing, screen printing, spray coating, or roll coating, but is not limited thereto.

The organic layer may be formed using a composition for an organic layer including the heterocyclic compound represented by Formula 1.

The composition for an organic layer of an organic light-emitting device according to the present disclosure may include the heterocyclic compound represented by Formula 1 and the compound represented by Formula 3. The two compounds may function as a dual host.

The organic layer may be formed by pre-mixing the heterocyclic compound represented by Formula 1 and the compound represented by Formula 3 and depositing the mixture by a thermal vacuum deposition method.

The ratio of the weight of the compound represented by Formula 3 to the weight of the heterocyclic compound represented by Formula 1, based on the total weight of the composition for an organic layer, may be 0.1 to 10. In some embodiments, the ratio of the weight of the compound represented by Formula 3 to the weight of the heterocyclic compound represented by Formula 1, based on the total weight of the composition for an organic layer, may be 0.2 to 5, or 0.5 to 2.

The composition may be used in forming the organic layer of an organic light-emitting device, and in particular, preferably used as a host material for the light-emitting layer.

The composition is in a form in which two or more compounds are simply mixed, and may be prepared by mixing powdered materials prior to forming the organic layer of an organic light-emitting device, or by mixing compounds that are liquid at a temperature above a predetermined temperature. The composition is in a solid state at or below the melting point of each material and may be maintained in a liquid state under high-temperature conditions.

The composition may further include materials known in the art, such as solvents and additives.

Hereinafter, embodiments of the present invention will be further described with reference to specific experimental examples. The examples and comparative examples included in the experimental examples are merely illustrative of the present disclosure and do not limit the scope of the appended claims. It will be apparent to those skilled in the art that various changes and modifications to the examples may be made within the scope and technical spirit of the present disclosure, and it is also understood that such changes and modifications fall within the scope of the appended claims.

### Preparative Example 1

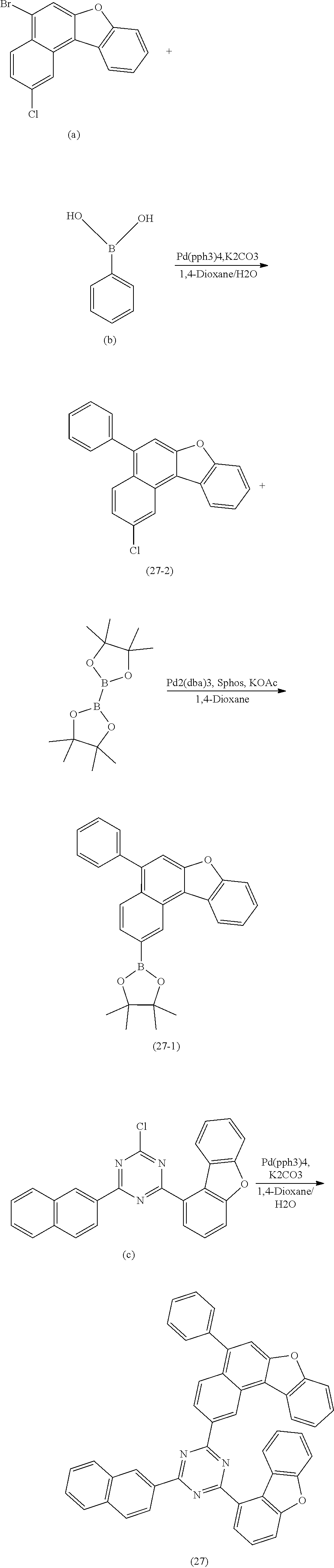

### 1) Preparation of Compound 27-2

10.0 g (30.16 mmol) of 5-bromo-2-chloronaphtho[2,1-b]benzofuran (a), 4.04 g (33.17 mmol) of phenylboronic acid (b), 1.74 g (1.508 mmol) of Pd(PPh₃)₄, and 8.34 g (60.32 mmol) of potassium carbonate were dissolved in a mixed solvent of 1,4-dioxane and H2O (100 ml/20 ml), and the mixture was refluxed at 110° C. for 4 hours. After completion of the reaction, distilled water and dichloromethane (DCM) were added at room temperature, and the mixture was extracted. The organic layer was dried over MgSO₄, and the solvent was removed using a rotary evaporator. The reaction product was purified by column chromatography (DCM/hexane=1:2) to obtain 8.0 g (80.6%) of the desired Compound 27-2.

### 2) Preparation of Compound 27-1

8 g (24.33 mmol) of Compound 27-2, 7.4 g (29.20 mmol) of 4,4,4′,4,5,5,5′,5′-octamethyl-2,2′-bi(1,3,2-dioxaborolane), 0.25 g (1.22 mmol) of Pd2(dba)3, 0.99 g (2.43 mmol) of Sphos, and 4.77 g (48.66 mmol) of potassium acetate were dissolved in 80 ml of 1,4-dioxane, and the mixture was refluxed at 110° C. for 3 hours. After completion of the reaction, distilled water and DCM were added at room temperature, and the mixture was extracted. The organic layer was dried over MgSO4 and the solvent was removed using a rotary evaporator. The reaction product was purified by column chromatography (DCM/hexane=1:2) to obtain 8.5 g (83.17%) of the desired Compound 27-1.

### 3) Preparation of Compound 27

8.5 g (20.22 mmol) of Compound 27-1, 8.23 g (20.22 mmol) of 2-chloro-4-(dibenzo[b,d]furan-1-yl)-6-(naphthalen-2-yl)-1,3,5-triazine (c), 1.17 g (1.01 mmol) of Pd(PPh3)4, and 5.59 g (40.44 mmol) of potassium carbonate were dissolved in a mixed solvent of 1,4-dioxane and H2O (85 ml/17 ml), and the mixture was refluxed at 110° C. for 6 hours. After completion of the reaction, distilled water and DCM were added at room temperature, and the mixture was extracted. The organic layer was dried over MgSO4 the solvent was removed using a rotary evaporator. The reaction product was purified by column chromatography (DCM) to obtain 10 g of the desired Compound 27 (74%).

Each desired compound was synthesized in the same manner as in Preparative Example 1, except that reaction products (a), (b), and (c) shown in Table 1 below were used.

TABLE 1

| | | | | Trans- |
|---|---|---|---|---|
| Com- | | | | ference |
| pound | (a) | (b) | (c) | number |
| 7 | 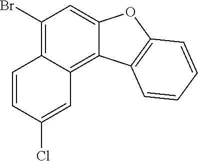 [SMILES: Clc1ccc2c(Br)cc3oc4ccccc4c3c2c1] | 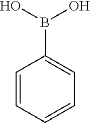 | 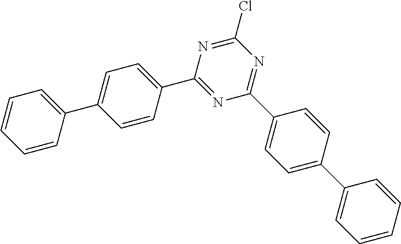 [SMILES: Clc1nc(-c2ccc(-c3ccccc3)cc2)nc(-c2ccc(-c3ccccc3)cc2)n1] | 80% |
| 9 | 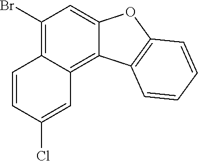 [SMILES: Clc1ccc2c(Br)cc3oc4ccccc4c3c2c1] | 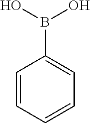 | 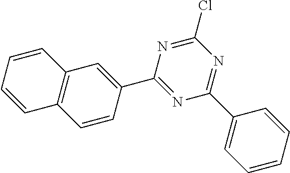 [SMILES: Clc1nc(-c2ccccc2)nc(-c2ccc3ccccc3c2)n1] | 77% |
| 20 | 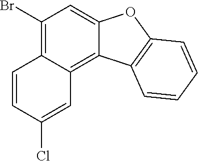 [SMILES: Clc1ccc2c(Br)cc3oc4ccccc4c3c2c1] | 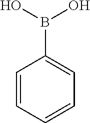 | 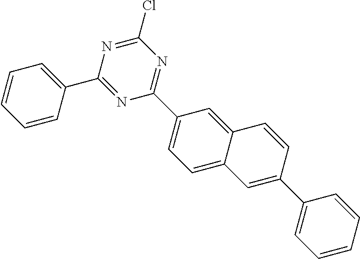 [SMILES: Clc1nc(-c2ccccc2)nc(-c2ccc3cc(-c4ccccc4)ccc3c2)n1] | 70% |
| 21 | 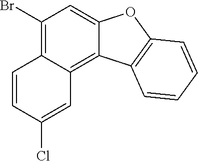 [SMILES: Clc1ccc2c(Br)cc3oc4ccccc4c3c2c1] | 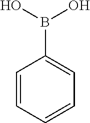 | 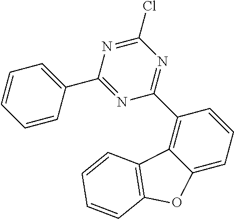 [SMILES: Clc1nc(-c2ccccc2)nc(-c2cccc3oc4ccccc4c23)n1] | 83% |
| 27 | 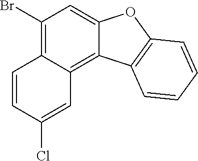 [SMILES: Clc1ccc2c(Br)cc3oc4ccccc4c3c2c1] | 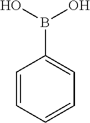 | 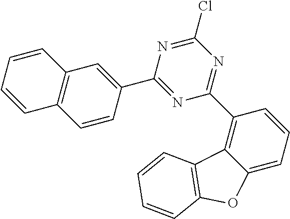 [SMILES: Clc1nc(-c2ccc3ccccc3c2)nc(-c2cccc3oc4ccccc4c23)n1] | 74% |
| 30 | 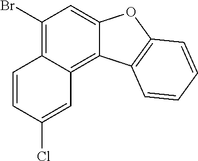 [SMILES: Clc1ccc2c(Br)cc3oc4ccccc4c3c2c1] | 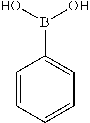 | 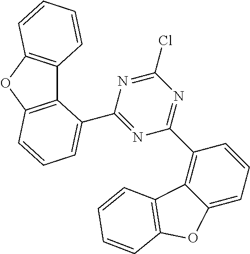 [SMILES: Clc1nc(-c2cccc3oc4ccccc4c23)nc(-c2cccc3oc4ccccc4c23)n1] | 75% |
| 35 | 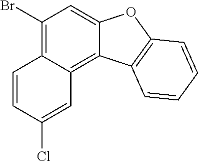 [SMILES: Clc1ccc2c(Br)cc3oc4ccccc4c3c2c1] | 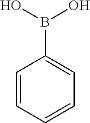 | 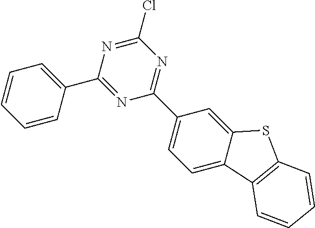 [SMILES: Clc1nc(-c2ccccc2)nc(-c2ccc3c(c2)sc2ccccc23)n1] | 76% |
| 44 | 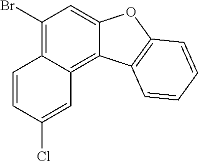 [SMILES: Clc1ccc2c(Br)cc3oc4ccccc4c3c2c1] | 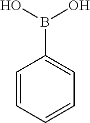 | 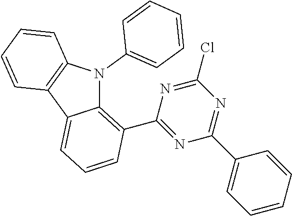 [SMILES: Clc1nc(-c2ccccc2)nc(-c2cccc3c4ccccc4n(-c4ccccc4)c23)n1] | 81% |
| 47 | 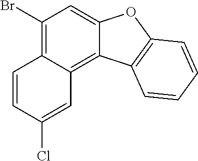 [SMILES: Clc1ccc2c(Br)cc3oc4ccccc4c3c2c1] | 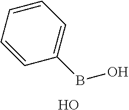 | 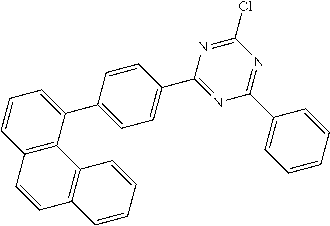 [SMILES: Clc1nc(-c2ccccc2)nc(-c2ccc(-c3cccc4ccc5ccccc5c34)cc2)n1] | 72% |
| 50 | 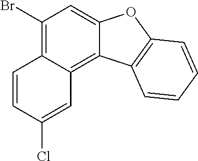 [SMILES: Clc1ccc2c(Br)cc3oc4ccccc4c3c2c1] | 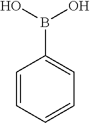 | 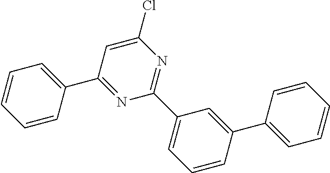 [SMILES: Clc1cc(-c2ccccc2)nc(-c2cccc(-c3ccccc3)c2)n1] | 80% |
| 61 | 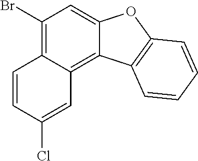 [SMILES: Clc1ccc2c(Br)cc3oc4ccccc4c3c2c1] | 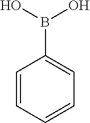 | 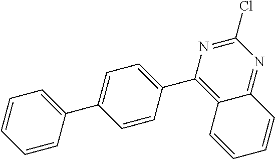 [SMILES: Clc1nc(-c2ccc(-c3ccccc3)cc2)c2ccccc2n1] | 61% |
| 65 | 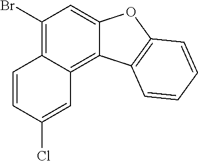 [SMILES: Clc1ccc2c(Br)cc3oc4ccccc4c3c2c1] | 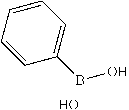 | 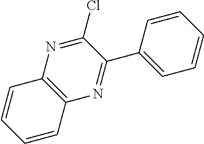 [SMILES: Clc1nc2ccccc2nc1-c1ccccc1] | 68% |
| 70 | 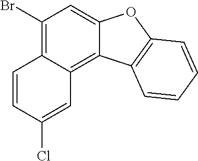 [SMILES: Clc1ccc2c(Br)cc3oc4ccccc4c3c2c1] | 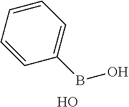 | 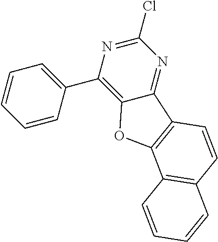 [SMILES: Clc1nc(-c2ccccc2)c2oc3c4ccccc4ccc3c2n1] | 70% |
| 74 | 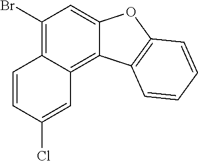 [SMILES: Clc1ccc2c(Br)cc3oc4ccccc4c3c2c1] | 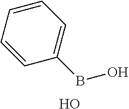 | 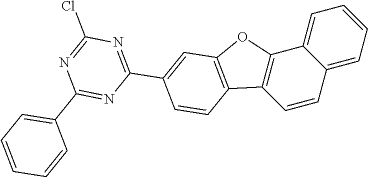 [SMILES: Clc1nc(-c2ccccc2)nc(-c2ccc3c(c2)oc2c4ccccc4ccc32)n1] | 74% |
| 77 | 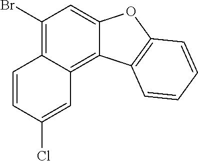 [SMILES: Clc1ccc2c(Br)cc3oc4ccccc4c3c2c1] | 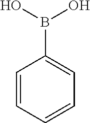 | 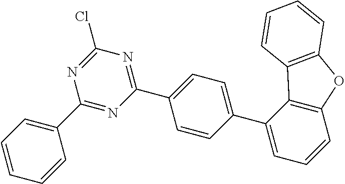 [SMILES: Clc1nc(-c2ccccc2)nc(-c2ccc(-c3cccc4oc5ccccc5c34)cc2)n1] | 77% |
| 81 | 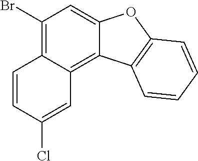 [SMILES: Clc1ccc2c(Br)cc3oc4ccccc4c3c2c1] | 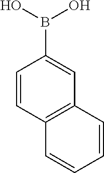 [SMILES: OB(O)c1ccc2ccccc2c1] | 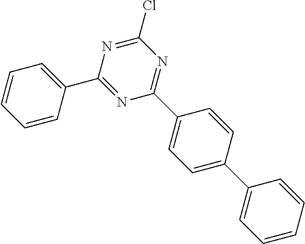 [SMILES: Clc1nc(-c2ccccc2)nc(-c2ccc(-c3ccccc3)cc2)n1] | 63% |
| 99 | 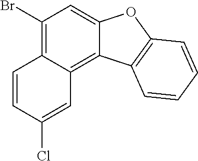 [SMILES: Clc1ccc2c(Br)cc3oc4ccccc4c3c2c1] | 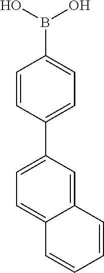 [SMILES: OB(O)c1ccc(-c2ccc3ccccc3c2)cc1] | 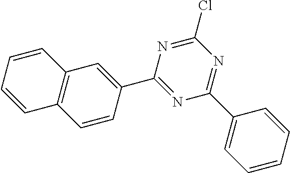 [SMILES: Clc1nc(-c2ccccc2)nc(-c2ccc3ccccc3c2)n1] | 70% |
| 127 | 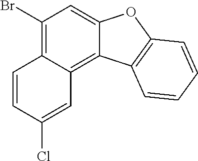 [SMILES: Clc1ccc2c(Br)cc3oc4ccccc4c3c2c1] | 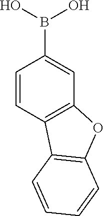 [SMILES: OB(O)c1ccc2c(c1)oc1ccccc12] | 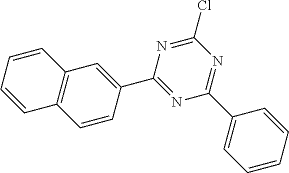 [SMILES: Clc1nc(-c2ccccc2)nc(-c2ccc3ccccc3c2)n1] | 81% |
| 137 | 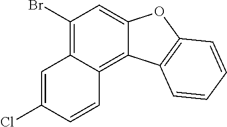 [SMILES: Clc1ccc2c(c1)c(Br)cc1oc3ccccc3c12] | 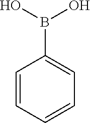 | 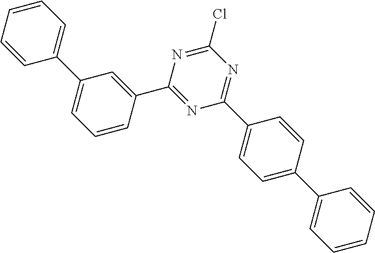 [SMILES: Clc1nc(-c2ccc(-c3ccccc3)cc2)nc(-c2cccc(-c3ccccc3)c2)n1] | 73% |
| 175 | 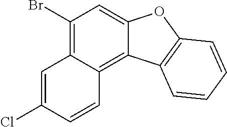 [SMILES: Clc1ccc2c(c1)c(Br)cc1oc3ccccc3c12] | 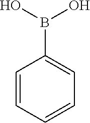 | 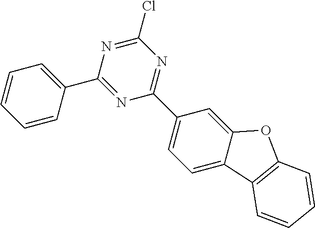 [SMILES: Clc1nc(-c2ccccc2)nc(-c2ccc3c(c2)oc2ccccc23)n1] | 72% |
| 177 | 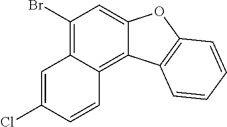 [SMILES: Clc1ccc2c(c1)c(Br)cc1oc3ccccc3c12] | 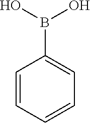 | 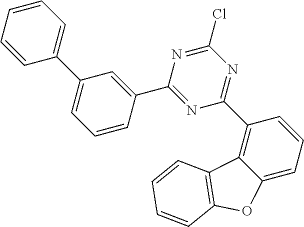 [SMILES: Clc1nc(-c2cccc(-c3ccccc3)c2)nc(-c2cccc3oc4ccccc4c23)n1] | 79% |
| 195 | 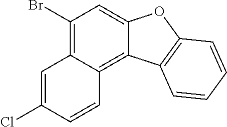 [SMILES: Clc1ccc2c(c1)c(Br)cc1oc3ccccc3c12] | 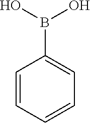 | 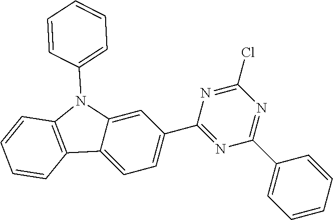 [SMILES: Clc1nc(-c2ccccc2)nc(-c2ccc3c4ccccc4n(-c4ccccc4)c3c2)n1] | 72% |
| 198 | 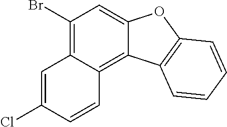 [SMILES: Clc1ccc2c(c1)c(Br)cc1oc3ccccc3c12] | 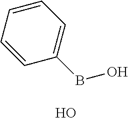 | 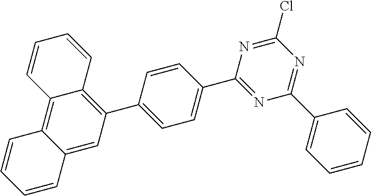 [SMILES: Clc1nc(-c2ccccc2)nc(-c2ccc(-c3cc4ccccc4c4ccccc34)cc2)n1] | 80% |
| 205 | 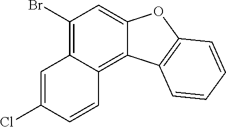 [SMILES: Clc1ccc2c(c1)c(Br)cc1oc3ccccc3c12] | 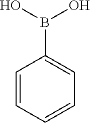 | 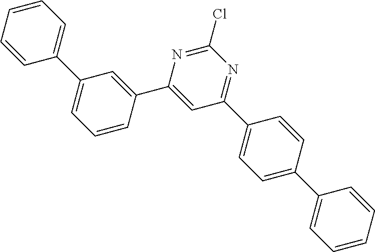 [SMILES: Clc1nc(-c2ccc(-c3ccccc3)cc2)cc(-c2cccc(-c3ccccc3)c2)n1] | 69% |
| 219 | 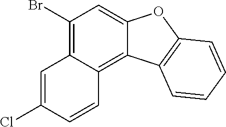 [SMILES: Clc1ccc2c(c1)c(Br)cc1oc3ccccc3c12] | 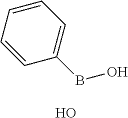 | 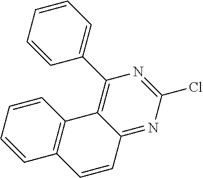 [SMILES: Clc1nc(-c2ccccc2)c2c(ccc3ccccc32)n1] | 70% |
| 220 | 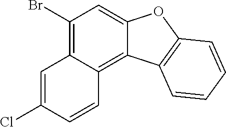 [SMILES: Clc1ccc2c(c1)c(Br)cc1oc3ccccc3c12] | 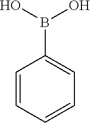 | 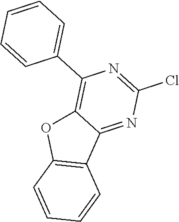 [SMILES: Clc1nc(-c2ccccc2)c2oc3ccccc3c2n1] | 70% |
| 222 | 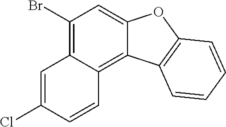 [SMILES: Clc1ccc2c(c1)c(Br)cc1oc3ccccc3c12] | 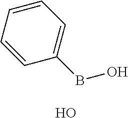 | 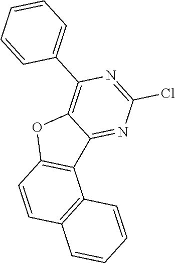 [SMILES: Clc1nc(-c2ccccc2)c2oc3ccc4ccccc4c3c2n1] | 81% |
| 228 | 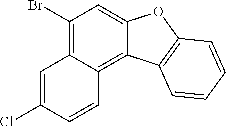 [SMILES: Clc1ccc2c(c1)c(Br)cc1oc3ccccc3c12] | 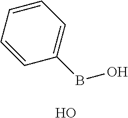 | 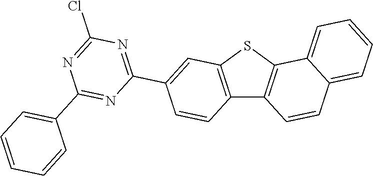 [SMILES: Clc1nc(-c2ccccc2)nc(-c2ccc3c(c2)sc2c4ccccc4ccc32)n1] | 77% |
| 230 | 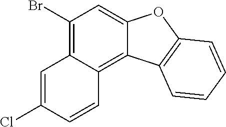 [SMILES: Clc1ccc2c(c1)c(Br)cc1oc3ccccc3c12] | 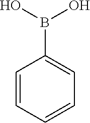 | 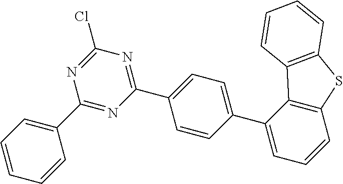 [SMILES: Clc1nc(-c2ccccc2)nc(-c2ccc(-c3cccc4sc5ccccc5c34)cc2)n1] | 83% |
| 258 | 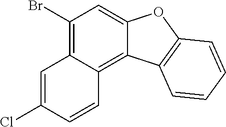 [SMILES: Clc1ccc2c(c1)c(Br)cc1oc3ccccc3c12] | 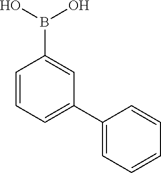 [SMILES: OB(O)c1cccc(-c2ccccc2)c1] | 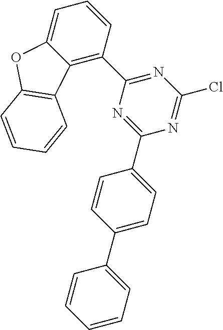 [SMILES: Clc1nc(-c2ccc(-c3ccccc3)cc2)nc(-c2cccc3oc4ccccc4c23)n1] | 75% |
| 280 | 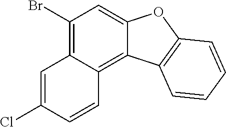 [SMILES: Clc1ccc2c(c1)c(Br)cc1oc3ccccc3c12] | 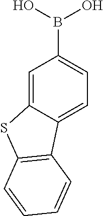 [SMILES: OB(O)c1ccc2c(c1)sc1ccccc12] | 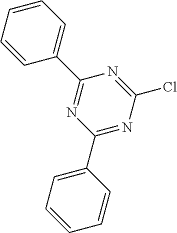 [SMILES: Clc1nc(-c2ccccc2)nc(-c2ccccc2)n1] | 66% |
| 294 | 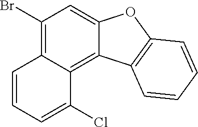 [SMILES: Clc1cccc2c(Br)cc3oc4ccccc4c3c12] | 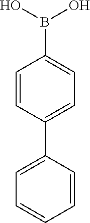 [SMILES: OB(O)c1ccc(-c2ccccc2)cc1] | 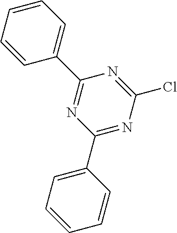 [SMILES: Clc1nc(-c2ccccc2)nc(-c2ccccc2)n1] | 68% |
| 299 | 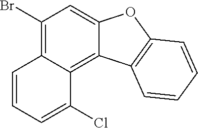 [SMILES: Clc1cccc2c(Br)cc3oc4ccccc4c3c12] | 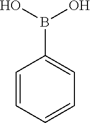 | 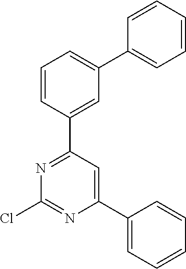 [SMILES: Clc1nc(-c2ccccc2)cc(-c2cccc(-c3ccccc3)c2)n1] | 68% |
| 301 | 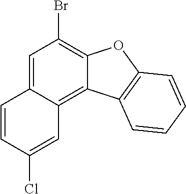 [SMILES: Clc1ccc2cc(Br)c3oc4ccccc4c3c2c1] | 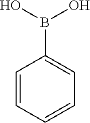 | 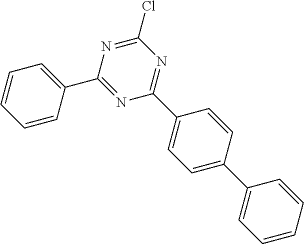 [SMILES: Clc1nc(-c2ccccc2)nc(-c2ccc(-c3ccccc3)cc2)n1] | 74% |
| 304 | 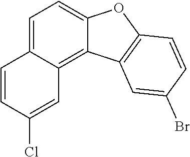 [SMILES: Clc1ccc2ccc3oc4ccc(Br)cc4c3c2c1] | 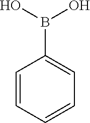 | 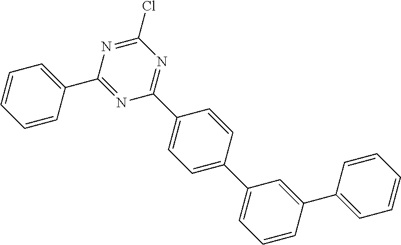 [SMILES: Clc1nc(-c2ccccc2)nc(-c2ccc(-c3cccc(-c4ccccc4)c3)cc2)n1] | 66% |
| 307 | 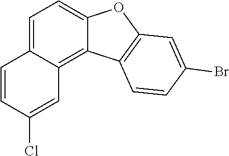 [SMILES: Clc1ccc2ccc3oc4cc(Br)ccc4c3c2c1] | 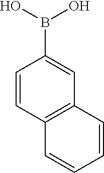 [SMILES: OB(O)c1ccc2ccccc2c1] | 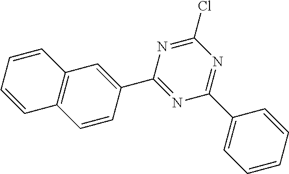 [SMILES: Clc1nc(-c2ccccc2)nc(-c2ccc3ccccc3c2)n1] | 70% |
| 319 | 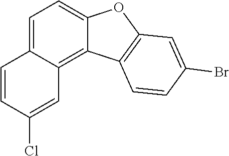 [SMILES: Clc1ccc2ccc3oc4cc(Br)ccc4c3c2c1] | 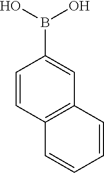 [SMILES: OB(O)c1ccc2ccccc2c1] | 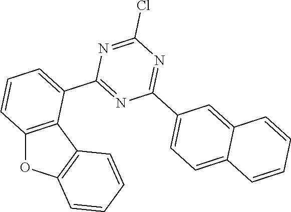 [SMILES: Clc1nc(-c2ccc3ccccc3c2)nc(-c2cccc3oc4ccccc4c23)n1] | 71% |
| 331 | 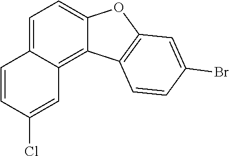 [SMILES: Clc1ccc2ccc3oc4cc(Br)ccc4c3c2c1] | 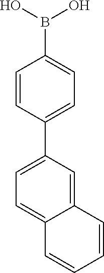 [SMILES: OB(O)c1ccc(-c2ccc3ccccc3c2)cc1] | 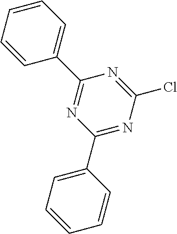 [SMILES: Clc1nc(-c2ccccc2)nc(-c2ccccc2)n1] | 74% |
| 346 | 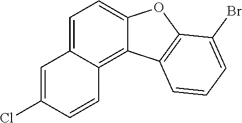 [SMILES: Clc1ccc2c(ccc3oc4c(Br)cccc4c32)c1] | 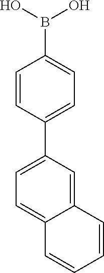 [SMILES: OB(O)c1ccc(-c2ccc3ccccc3c2)cc1] | 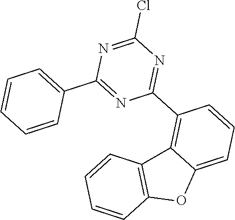 [SMILES: Clc1nc(-c2ccccc2)nc(-c2cccc3oc4ccccc4c23)n1] | 77% |
| 371 | 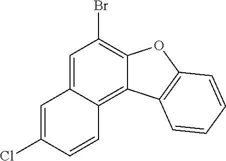 [SMILES: Clc1ccc2c(c1)cc(Br)c1oc3ccccc3c12] | 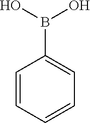 | 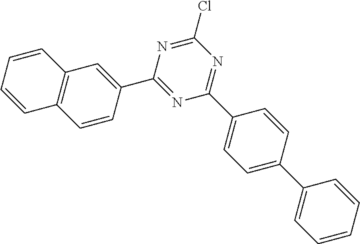 [SMILES: Clc1nc(-c2ccc(-c3ccccc3)cc2)nc(-c2ccc3ccccc3c2)n1] | 80% |
| 377 | 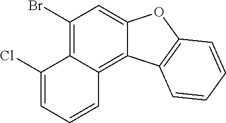 [SMILES: Clc1cccc2c1c(Br)cc1oc3ccccc3c12] | 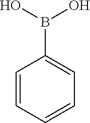 | 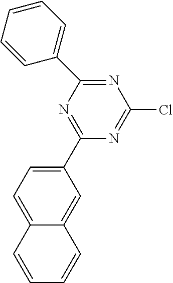 [SMILES: Clc1nc(-c2ccccc2)nc(-c2ccc3ccccc3c2)n1] | 83% |
| 387 | 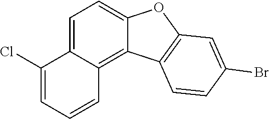 [SMILES: Clc1cccc2c1ccc1oc3cc(Br)ccc3c12] | 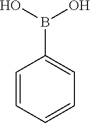 | 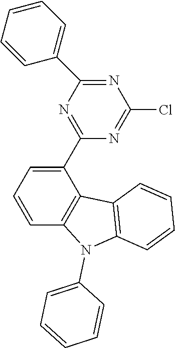 [SMILES: Clc1nc(-c2ccccc2)nc(-c2cccc3c2c2ccccc2n3-c2ccccc2)n1] | 75% |
| 390 | 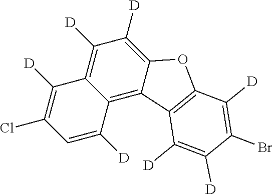 [SMILES: [2H]c1c(Br)c([2H])c2oc3c([2H])c([2H])c4c([2H])c(Cl)cc([2H])c4c3c2c1[2H]] | 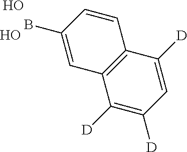 | 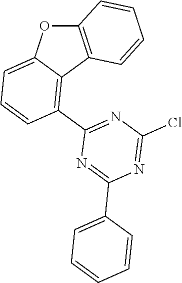 [SMILES: Clc1nc(-c2ccccc2)nc(-c2cccc3oc4ccccc4c23)n1] | 71% |
| 396 | 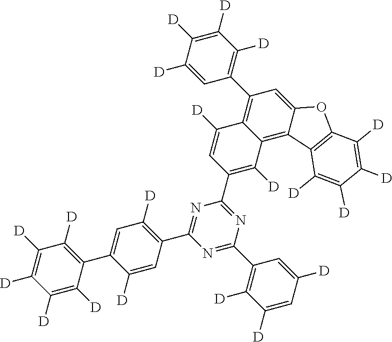 [SMILES: [2H]c1cc([2H])c([2H])c(-c2nc(-c3cc([2H])c(-c4c([2H])c([2H])c([2H])c([2H])c4[2H])cc3[2H])nc(-c3cc([2H])c4c(-c5cc([2H])c([2H])c([2H])c5[2H])cc5oc6c([2H])c([2H])c([2H])c([2H])c6c5c4c3[2H])n2)c1] | 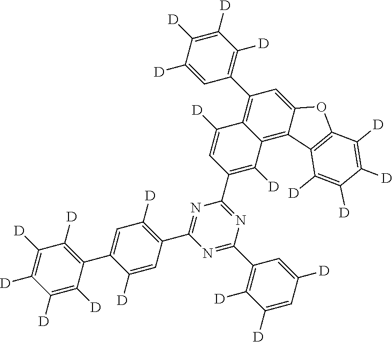 [SMILES: [2H]c1cc([2H])c([2H])c(-c2nc(-c3cc([2H])c(-c4c([2H])c([2H])c([2H])c([2H])c4[2H])cc3[2H])nc(-c3cc([2H])c4c(-c5cc([2H])c([2H])c([2H])c5[2H])cc5oc6c([2H])c([2H])c([2H])c([2H])c6c5c4c3[2H])n2)c1] | 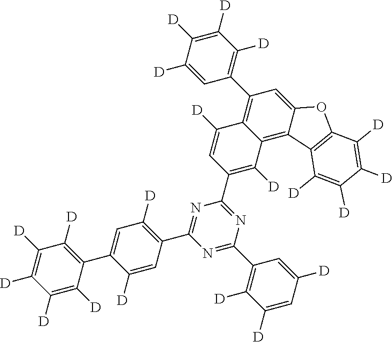 [SMILES: [2H]c1cc([2H])c([2H])c(-c2nc(-c3cc([2H])c(-c4c([2H])c([2H])c([2H])c([2H])c4[2H])cc3[2H])nc(-c3cc([2H])c4c(-c5cc([2H])c([2H])c([2H])c5[2H])cc5oc6c([2H])c([2H])c([2H])c([2H])c6c5c4c3[2H])n2)c1] | 70% |

### Preparative Example 2

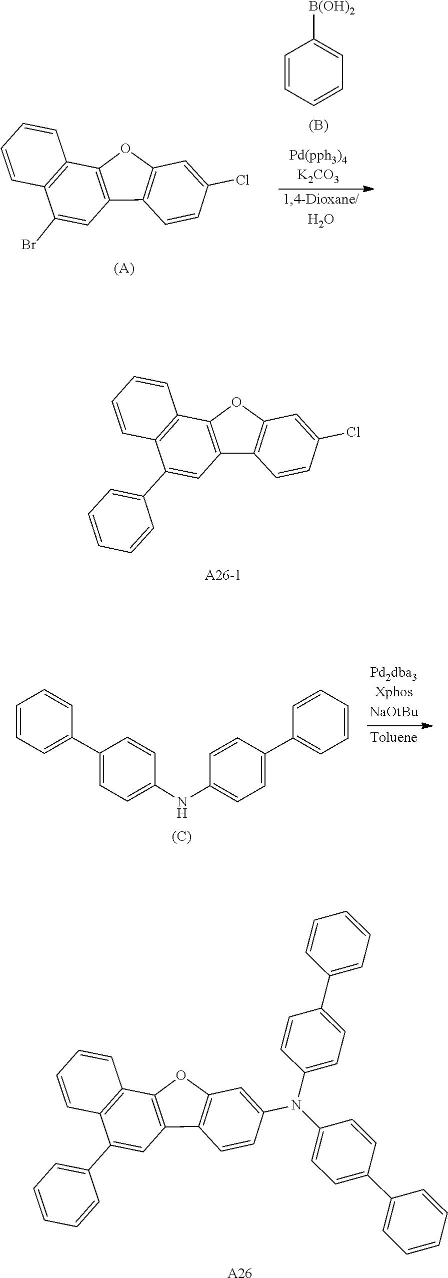

### 1) Preparation of Intermediate A26-1

In a 1 L two-necked flask, 30.0 g (90.5 mmol) of 5-bromo-9-chloronaphtho[1,2-b]benzofuran (A), 12.1 g (99.6 mmol) of phenylboronic acid (B), 5.2 g (4.5 mmol) of Pd(PPh3) 4, and 25.0 g (181.0 mmol) of K2CO3 were dissolved in a mixed solvent of 1,4-dioxane and H2O (300 ml/60 ml), and the mixture was refluxed for 1 hour. The reaction product was purified by recrystallization from methanol to obtain 24.8 g (83% yield) of Compound A26-1.

### 2) Preparation of Compound A26

In a 500 ml two-necked flask, 10.0 g (30.4 mmol) of Compound A26-1, 11.3 g (30.4 mmol) of di([1,1′-biphenyl]-4-yl)amine (C), 1.4 g (1.5 mmol) of Pd2(dba)3, 1.4 g (3.0 mmol) of Xphos, and 5.8 g (60.8 mmol) of NaOtBu were dissolved in toluene (150 ml), and the mixture was refluxed for 1 hour. The reaction product was purified by recrystallization from methanol to obtain 17.6 g (87% yield) of the desired Compound A26.

Each desired compound was synthesized in the same manner as in Preparative Example 2, except that reaction products (A), (B), and (C) shown in Table 2 below were used.

TABLE 2

| | | | | Trans- |
|---|---|---|---|---|
| Com- | | | | ference |
| pound | (A) | (B) | (C) | number |
| A1 | 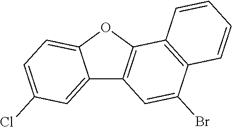 [SMILES: Clc1ccc2oc3c4ccccc4c(Br)cc3c2c1] | 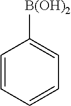 | 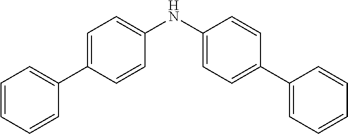 [SMILES: c1ccc(-c2ccc(Nc3ccc(-c4ccccc4)cc3)cc2)cc1] | 78% |
| A3 | 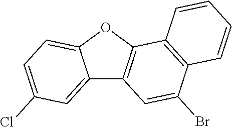 [SMILES: Clc1ccc2oc3c4ccccc4c(Br)cc3c2c1] | 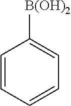 | 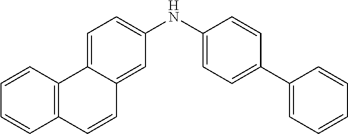 [SMILES: c1ccc(-c2ccc(Nc3ccc4c(ccc5ccccc54)c3)cc2)cc1] | 77% |
| A11 | 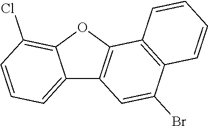 [SMILES: Clc1cccc2c1oc1c3ccccc3c(Br)cc21] | 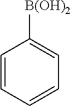 | 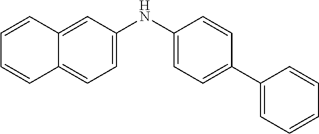 [SMILES: c1ccc(-c2ccc(Nc3ccc4ccccc4c3)cc2)cc1] | 70% |
| A16 | 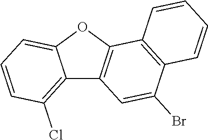 [SMILES: Clc1cccc2oc3c4ccccc4c(Br)cc3c12] | 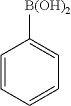 | 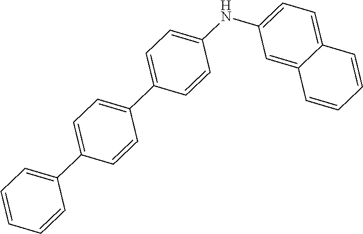 [SMILES: c1ccc(-c2ccc(-c3ccc(Nc4ccc5ccccc5c4)cc3)cc2)cc1] | 79% |
| A21 | 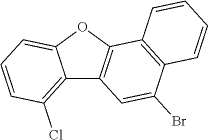 [SMILES: Clc1cccc2oc3c4ccccc4c(Br)cc3c12] | 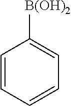 | 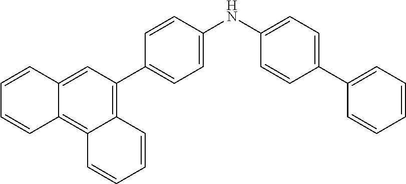 [SMILES: c1ccc(-c2ccc(Nc3ccc(-c4cc5ccccc5c5ccccc45)cc3)cc2)cc1] | 72% |
| A26 | 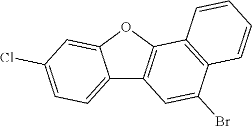 [SMILES: Clc1ccc2c(c1)oc1c3ccccc3c(Br)cc21] | 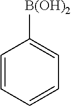 | 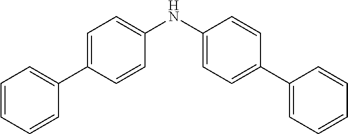 [SMILES: c1ccc(-c2ccc(Nc3ccc(-c4ccccc4)cc3)cc2)cc1] | 76% |
| A29 | 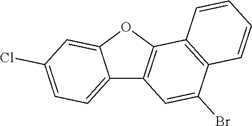 [SMILES: Clc1ccc2c(c1)oc1c3ccccc3c(Br)cc21] | 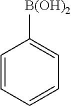 | 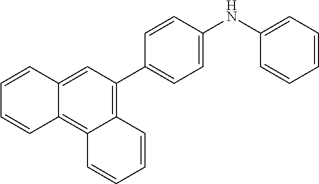 [SMILES: c1ccc(Nc2ccc(-c3cc4ccccc4c4ccccc34)cc2)cc1] | 77% |
| A41 | 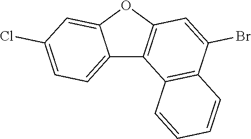 [SMILES: Clc1ccc2c(c1)oc1cc(Br)c3ccccc3c12] | 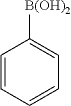 | 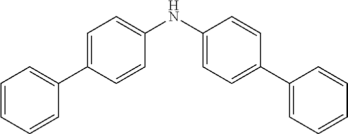 [SMILES: c1ccc(-c2ccc(Nc3ccc(-c4ccccc4)cc3)cc2)cc1] | 76% |
| A66 | 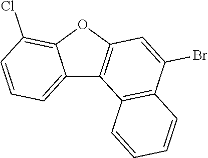 [SMILES: Clc1cccc2c1oc1cc(Br)c3ccccc3c12] | 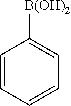 | 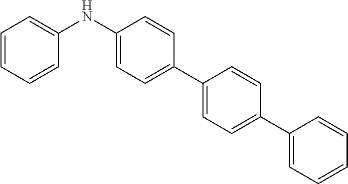 [SMILES: c1ccc(Nc2ccc(-c3ccc(-c4ccccc4)cc3)cc2)cc1] | 72% |
| A69 | 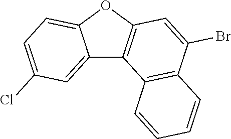 [SMILES: Clc1ccc2oc3cc(Br)c4ccccc4c3c2c1] | 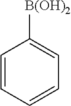 | 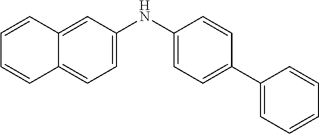 [SMILES: c1ccc(-c2ccc(Nc3ccc4ccccc4c3)cc2)cc1] | 75% |
| A74 | 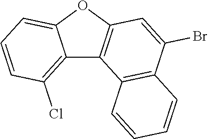 [SMILES: Clc1cccc2oc3cc(Br)c4ccccc4c3c12] | 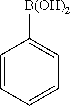 | 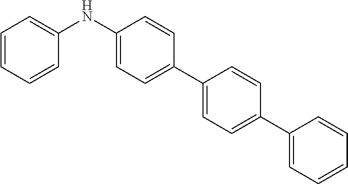 [SMILES: c1ccc(Nc2ccc(-c3ccc(-c4ccccc4)cc3)cc2)cc1] | 73% |
| A81 | 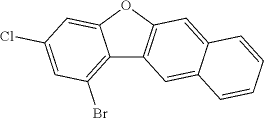 [SMILES: Clc1cc(Br)c2c(c1)oc1cc3ccccc3cc12] | 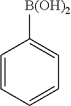 | 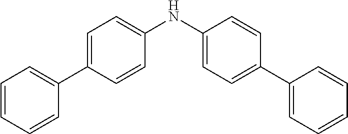 [SMILES: c1ccc(-c2ccc(Nc3ccc(-c4ccccc4)cc3)cc2)cc1] | 75% |
| A99 | 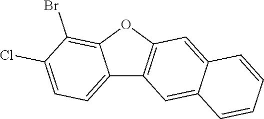 [SMILES: Clc1ccc2c(oc3cc4ccccc4cc32)c1Br] | 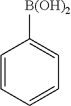 | 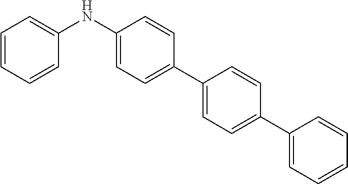 [SMILES: c1ccc(Nc2ccc(-c3ccc(-c4ccccc4)cc3)cc2)cc1] | 72% |

### Preparative Example 3

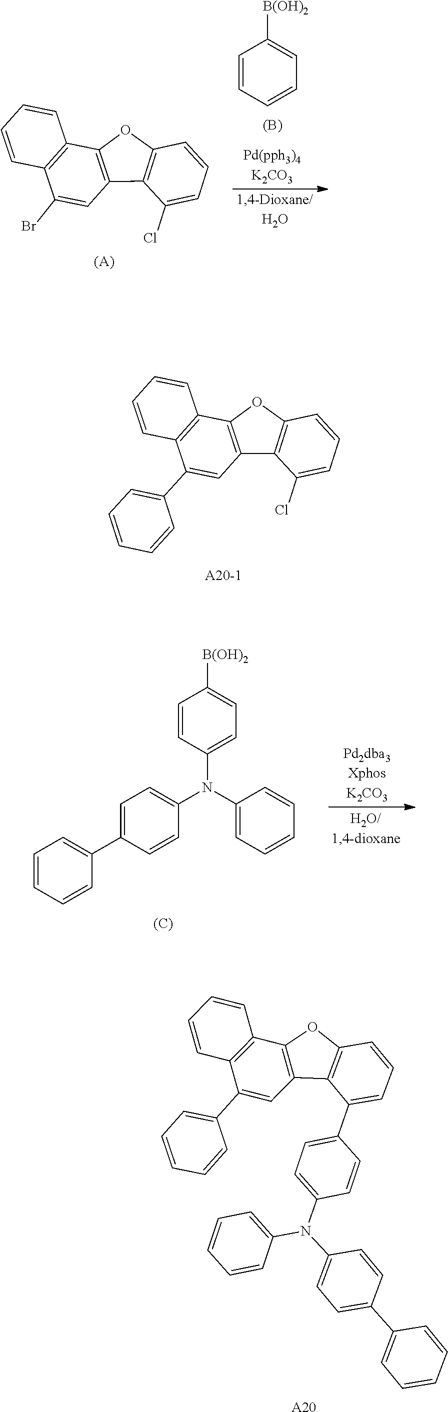

### 1) Preparation of Intermediate A20-1

In a 1 L two-necked flask, 30.0 g (90.5 mmol) of 5-bromo-7-chloronaphtho[1,2-b]benzofuran (A), 12.1 g (99.6 mmol) of phenylboronic acid (B), 5.2 g (4.5 mmol) of Pd(PPh3) 4, and 25.0 g (181.0 mmol) of K2CO3 were dissolved in a mixed solvent of 1,4-dioxane and H2O (300 ml/60 ml), and the mixture was refluxed for 1 hour. The reaction product was purified by recrystallization from methanol to obtain 24.8 g (83.3% yield) of Compound A20-1.

### 2) Preparation of Compound A20

In a 250 ml two-necked flask, 10.0 g (30.4 mmol) of Compound A20-1, 11.1 g (30.4 mmol) of (4-[1,1′-biphenyl]-4-yl(phenyl)amino)phenyl) boronic acid (C), 1.4 g (1.5 mmol) of Pd2(dba)3, 1.4 g (3.0 mmol) of Xphos, and 8.4 g (60.8 mmol) of K2CO3 were dissolved in a mixed solvent of 1,4-dioxane and H2O (100 ml/20 ml), and the mixture was refluxed for 1 hour. The reaction product was purified by recrystallization from methanol to obtain 15.1 g (80.9% yield) of the desired Compound A20.

Each desired compound was synthesized in the same manner as in Preparative Example 3, except that reaction products (A), (B), and (C) shown in Table 3 below were used

TABLE 3

| | | | | Trans- |
|---|---|---|---|---|
| | | | | fer- |
| | | | | ence |
| Com- | | | | num- |
| pound | (A) | (B) | (C) | ber |
| A4 | 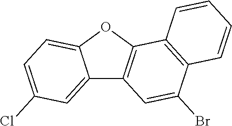 [SMILES: Clc1ccc2oc3c4ccccc4c(Br)cc3c2c1] | 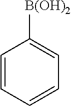 | 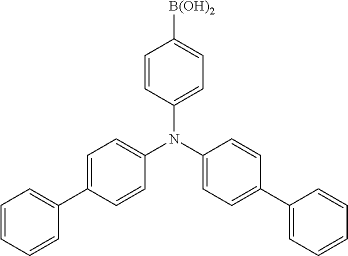 [SMILES: OB(O)c1ccc(N(c2ccc(-c3ccccc3)cc2)c2ccc(-c3ccccc3)cc2)cc1] | 78% |
| A7 | 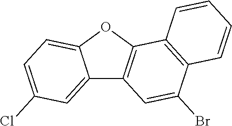 [SMILES: Clc1ccc2oc3c4ccccc4c(Br)cc3c2c1] | 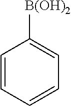 | 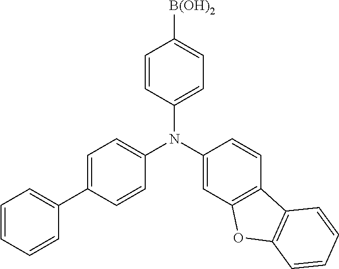 [SMILES: OB(O)c1ccc(N(c2ccc(-c3ccccc3)cc2)c2ccc3c(c2)oc2ccccc23)cc1] | 77% |
| A12 | 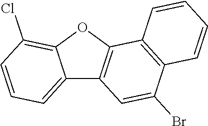 [SMILES: Clc1cccc2c1oc1c3ccccc3c(Br)cc21] | 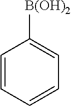 | 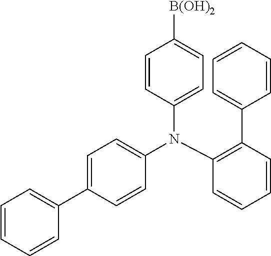 [SMILES: OB(O)c1ccc(N(c2ccc(-c3ccccc3)cc2)c2ccccc2-c2ccccc2)cc1] | 72% |
| A13 | 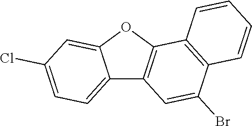 [SMILES: Clc1ccc2c(c1)oc1c3ccccc3c(Br)cc21] | 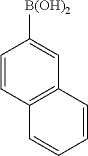 [SMILES: OB(O)c1ccc2ccccc2c1] | 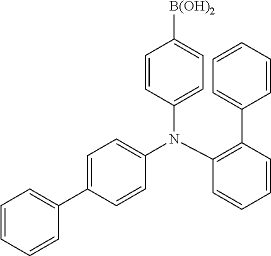 [SMILES: OB(O)c1ccc(N(c2ccc(-c3ccccc3)cc2)c2ccccc2-c2ccccc2)cc1] | 71% |
| A17 | 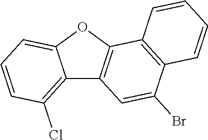 [SMILES: Clc1cccc2oc3c4ccccc4c(Br)cc3c12] | 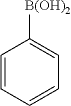 | 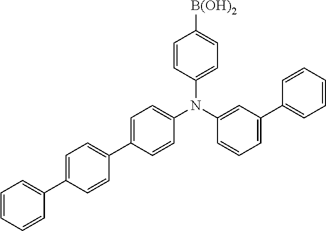 [SMILES: OB(O)c1ccc(N(c2ccc(-c3ccc(-c4ccccc4)cc3)cc2)c2cccc(-c3ccccc3)c2)cc1] | 73% |
| A20 | 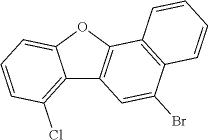 [SMILES: Clc1cccc2oc3c4ccccc4c(Br)cc3c12] | 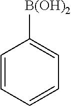 | 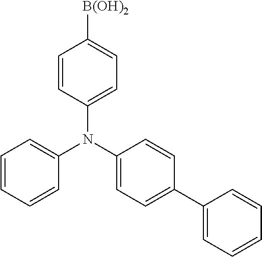 [SMILES: OB(O)c1ccc(N(c2ccccc2)c2ccc(-c3ccccc3)cc2)cc1] | 74% |
| A24 | 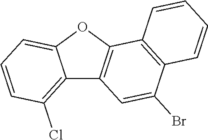 [SMILES: Clc1cccc2oc3c4ccccc4c(Br)cc3c12] | 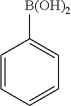 | 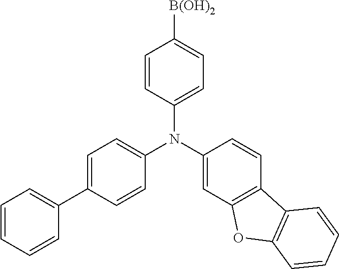 [SMILES: OB(O)c1ccc(N(c2ccc(-c3ccccc3)cc2)c2ccc3c(c2)oc2ccccc23)cc1] | 73% |
| A25 | 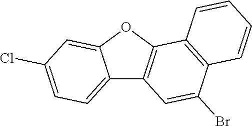 [SMILES: Clc1ccc2c(c1)oc1c3ccccc3c(Br)cc21] | 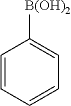 | 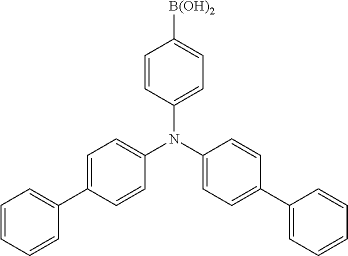 [SMILES: OB(O)c1ccc(N(c2ccc(-c3ccccc3)cc2)c2ccc(-c3ccccc3)cc2)cc1] | 75% |
| A37 | 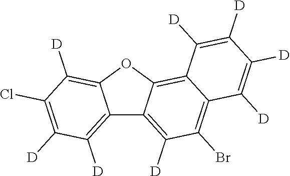 [SMILES: [2H]c1c([2H])c([2H])c2c(c1[2H])c(Br)c([2H])c1c2oc2c([2H])c(Cl)c([2H])c([2H])c21] | 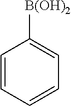 | 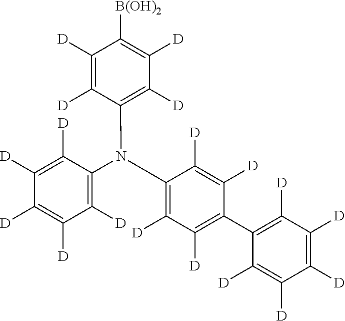 [SMILES: [2H]c1c([2H])c([2H])c(-c2c([2H])c([2H])c(N(c3c([2H])c([2H])c([2H])c([2H])c3[2H])c3c([2H])c([2H])c(B(O)O)c([2H])c3[2H])c([2H])c2[2H])c([2H])c1[2H]] | 71% |
| A58 | 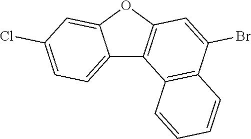 [SMILES: Clc1ccc2c(c1)oc1cc(Br)c3ccccc3c12] | 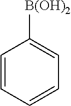 | 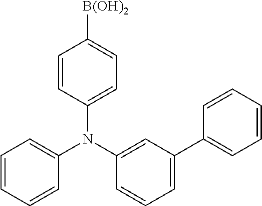 [SMILES: OB(O)c1ccc(N(c2ccccc2)c2cccc(-c3ccccc3)c2)cc1] | 76% |
| A78 | 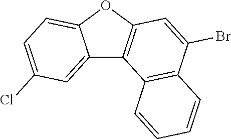 [SMILES: Clc1ccc2oc3cc(Br)c4ccccc4c3c2c1] | 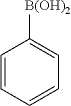 | 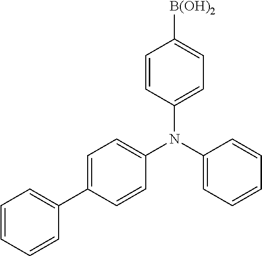 [SMILES: OB(O)c1ccc(N(c2ccccc2)c2ccc(-c3ccccc3)cc2)cc1] | 77% |
| A86 | 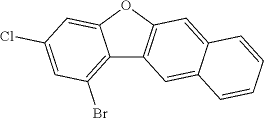 [SMILES: Clc1cc(Br)c2c(c1)oc1cc3ccccc3cc12] | 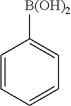 | 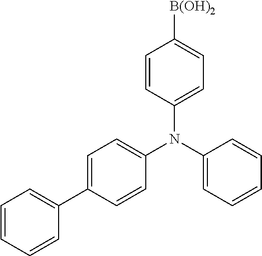 [SMILES: OB(O)c1ccc(N(c2ccccc2)c2ccc(-c3ccccc3)cc2)cc1] | 73% |

The synthesis results for the compounds in Tables 1 to 3 are shown in Tables 4 and 5 below. Table 4 shows the 1H NMR (400 MHz, CDCl3) measurements, and Table 5 shows the field-desorption mass spectrometry (FD-MS) measurements.

TABLE 4

| Compound | |
|---|---|
| number | ¹H NMR(CDCl₃, 400 Mz) |
| 7 | δ = 8.25(4H, s), 8.18(1H, d), 8.03(1H, d), 7.98(1H, d), 7.82(1H, d), 7.79(2H, d), 7.75(4H, |
| | d), 7.54(1H, d), 7.49(4H, d), 7.46(2H, dd), 7.41(3H, d), 7.39(1H, d), 7.31(2H, dd), |
| | 7.25(4H, dd) |
| 9 | δ = 9.09(1H, s), 8.49(1H, d), 8.36(2H, d), 8.18(1H, d), 8.16(1H, d), 8.08(1H, d), 8.03(1H, |
| | d), 8(1H, d), 7.98(1H, d), 7.82(1H, dd), 7.79(2H, d), 7.61(1H, d), 7.57(1H, dd), 7.54(1H, |
| | dd), 7.5(3H, s), 7.46 (2H, t), 7.41 (1H, t), 7.39 (1H, dd), 7.31 (2H, dd) |
| 20 | δ = 9.15(1H, s), 8.51(1H, d), 8.36(2H, d), 8.18(1H, d), 8.03(1H, d), 7.99(1H, d), 7.98(1H, |
| | d), 7.91(1H, d), 7.82(1H, d), 7.79(2H, dd), 7.75(2H, d), 7.55(1H, d), 7.54(1H, dd), 7.5(3H, |
| | dd), 7.49(2H, s), 7.46 (2H, t), 7.41 (2H, t), 7.39 (1H, dd), 7.38 (1H, dd), 7.31 (2H, t) |
| 21 | δ = 8.36(2H, s), 8.18(1H, d), 8.03(1H, d), 7.98(2H, d), 7.82(1H, d), 7.79(2H, d), 7.6(1H, |
| | d), 7.57(1H, d), 7.54(2H, d), 7.5(3H, dd), 7.47(1H, d), 7.46(2H, d), 7.41(1H, dd), 7.39(2H, |
| | dd), 7.31(3H, s) |
| 27 | δ = 9.09(1H, s), 8.49(1H, d), 8.18(1H, d), 8.16(1H, d), 8.08(1H, d), 8.03(1H, d), 8(1H, d), |
| | 7.98(2H, d), 7.82(1H, d), 7.79(2H, dd), 7.61(1H, d), 7.6(1H, d), 7.57(2H, dd), 7.54(2H, |
| | dd), 7.47(1H, s), 7.46 (2H, t), 7.41 (1H, t), 7.39 (2H, dd), 7.31 (3H, dd) |
| 30 | δ = 8.18(1H, s), 8.03(1H, d), 7.98(3H, d), 7.82(1H, d), 7.79(2H, d), 7.6(2H, d), 7.57(2H, |
| | d), 7.54(3H, d), 7.47(2H, d), 7.46(2H, dd), 7.41(1H, d), 7.39(3H, d), 7.31(4H, dd) |
| 35 | δ = 8.45(1H, s), 8.36(2H, d), 8.2(1H, d), 8.18(1H, d), 8.03(1H, d), 7.98(1H, d), 7.97(1H, |
| | d), 7.86(1H, d), 7.82(1H, d), 7.79(2H, dd), 7.67(1H, d), 7.56(1H, d), 7.54(1H, dd), 7.5(3H, |
| | dd), 7.46(2H, s), 7.41 (1H, t), 7.39 (1H, t), 7.31 (3H, dd) |
| 44 | δ = 8.65(1H, s), 8.36(2H, d), 8.19(1H, d), 8.18(1H, d), 8.03(1H, d), 7.98(1H, d), 7.87(1H, |
| | d), 7.82(1H, d), 7.79(2H, d), 7.62(2H, dd), 7.58(1H, d), 7.54(1H, d), 7.5(5H, dd), 7.46(2H, |
| | dd), 7.41(1H, s), 7.4 (1H, t), 7.39 (1H, t), 7.31 (2H, dd), 7.2 (1H, dd), 7.18 (1H, t), 7.16 (1H, |
| | d) |
| 47 | δ = 8.98(1H, s), 8.41(1H, d), 8.36(2H, d), 8.27(1H, d), 8.25(2H, d), 8.23(1H, d), 8.18(1H, |
| | d), 8.08(1H, d), 8.03(1H, d), 7.98(1H, dd), 7.9(1H, d), 7.82(1H, d), 7.79(2H, dd), 7.75(1H, |
| | dd), 7.68(1H, s), 7.63 (1H, t), 7.54 (1H, t), 7.5 (3H, dd), 7.46 (2H, dd), 7.39 (1H, t), 7.31 |
| | (2H, d), 7.25 (2H, d) |
| 50 | δ = 8.38(1H, s), 8.23(1H, d), 8.18(1H, d), 8.03(1H, d), 7.98(1H, d), 7.94(3H, d), 7.82(1H, |
| | d), 7.79(2H, d), 7.75(2H, d), 7.71(1H, dd), 7.61(1H, d), 7.55(2H, d), 7.54(1H, dd), |
| | 7.49(3H, dd), 7.46(2H, s), 7.41 (2H, t), 7.39 (1H, t), 7.31 (2H, dd) |
| 61 | δ = 8.3(2H, s), 8.18(1H, d), 8.13(1H, d), 8.03(1H, d), 7.98(1H, d), 7.85(2H, d), 7.84(1H, |
| | d), 7.83(1H, d), 7.82(1H, d), 7.79(2H, dd), 7.75(2H, d), 7.58(1H, d), 7.54(1H, dd), |
| | 7.49(2H, dd), 7.46(2H, s), 7.41 (2H, t), 7.39 (1H, t), 7.31 (2H, dd) |
| 65 | δ = 8.74(1H, s), 8.38(1H, d), 8.07(1H, d), 8.03(2H, d), 7.98(1H, d), 7.8(2H, d), 7.79(2H, |
| | d), 7.67(2H, d), 7.59(1H, d), 7.54(1H, dd), 7.46(2H, d), 7.41(1H, d), 7.39(1H, dd), |
| | 7.32(2H, dd), 7.31(2H, s) |
| 70 | δ = 8.19(1H, s), 8.18(1H, d), 8.03(1H, d), 7.98(1H, d), 7.84(3H, d), 7.82(1H, d), 7.79(2H, |
| | d), 7.59(1H, d), 7.54(1H, d), 7.53(2H, dd), 7.49(1H, d), 7.48(2H, d), 7.46(2H, dd), |
| | 7.41(1H, dd), 7.39(1H, s), 7.31 (2H, t), 7.23 (1H, t) |
| 74 | δ = 8.36(2H, s), 8.19(1H, d), 8.18(1H, d), 8.03(1H, d), 7.98(1H, d), 7.84(1H, d), 7.82(1H, |
| | d), 7.79(2H, d), 7.78(1H, d), 7.76(1H, dd), 7.59(1H, d), 7.57(1H, d), 7.54(1H, dd), 7.5(3H, |
| | dd), 7.48(2H, s), 7.46 (2H, t), 7.41 (1H, t), 7.39 (1H, dd), 7.31 (2H, dd), 7.23 (1H, t) |
| 77 | δ = 8.36(2H, s), 8.25(2H, d), 8.18(1H, d), 8.03(1H, d), 7.98(2H, d), 7.82(1H, d), 7.79(2H, |
| | d), 7.6(1H, d), 7.57(1H, d), 7.54(2H, dd), 7.5(3H, d), 7.47(1H, d), 7.46(2H, dd), 7.41(1H, |
| | dd), 7.39(2H, s), 7.31 (3H, t), 7.25 (2H, t) |
| 81 | δ = 8.97 (1H, s), 8.55 (1H, d), 8.36 (4H, d), 8.31 (1H, d), 8.12 (4H, d), 7.99 (1H, d), 7.94 |
| | (1H, d), 7.91 (1H, d), 7.74 (1H, d), 7.62 (2H, dd), 7.59 (2H, d), 7.58 (1H, d), 7.5 (8H, dd), |
| | 7.35 (1H, dd), 7.16 (1H, s) |
| 99 | δ = 9.09(1H, s), 8.49(1H, d), 8.36(2H, d), 8.18(1H, d), 8.16(1H, d), 8.09(1H, d), 8.08(1H, |
| | d), 8.06(1H, d), 8.03(1H, d), 8(1H, dd), 7.99(1H, d), 7.98(1H, d), 7.82(1H, dd), 7.63(1H, |
| | dd), 7.61(1H, s), 7.58 (1H, t), 7.57 (1H, t), 7.55 (1H, dd), 7.54 (1H, dd), 7.5 (3H, t), 7.39 |
| | (1H, d), 7.38 (1H, d), 7.31 (2H, d) |
| 127 | δ = 9.09(1H, s), 8.49(1H, d), 8.36(2H, d), 8.18(1H, d), 8.16(1H, d), 8.08(1H, d), 8.03(1H, |
| | d), 8(1H, d), 7.98(2H, d), 7.82(1H, dd), 7.78(1H, d), 7.76(1H, d), 7.61(1H, dd), 7.57(2H, |
| | dd), 7.54(2H, s), 7.5 (3H, t), 7.39 (2H, t), 7.31 (3H, dd) |
| 137 | δ = 8.38(1H, s), 8.25(2H, d), 8.18(1H, d), 8.03(1H, d), 7.98(1H, d), 7.94(1H, d), 7.82(1H, |
| | d), 7.79(2H, d), 7.75(4H, d), 7.71(1H, dd), 7.61(1H, d), 7.54(1H, d), 7.49(4H, dd), |
| | 7.46(2H, dd), 7.41(3H, s), 7.39 (1H, t), 7.31 (2H, t), 7.25 (2H, dd) |
| 175 | δ = 8.36(2H, s), 8.18(1H, d), 8.03(1H, d), 7.98(2H, d), 7.82(1H, d), 7.79(2H, d), 7.78(1H, |
| | d), 7.76(1H, d), 7.57(1H, d), 7.54(2H, dd), 7.5(3H, d), 7.46(2H, d), 7.41(1H, dd), 7.39(2H, |
| | dd), 7.31(3H, s) |
| 177 | δ = 8.38(1H, s), 8.18(1H, d), 8.03(1H, d), 7.98(2H, d), 7.94(1H, d), 7.82(1H, d), 7.79(2H, |
| | d), 7.75(2H, d), 7.71(1H, d), 7.61(1H, dd), 7.6(1H, d), 7.57(1H, d), 7.54(2H, dd), 7.49(2H, |
| | dd), 7.47(1H, s), 7.46 (2H, t), 7.41 (2H, t), 7.39 (2H, dd), 7.31 (3H, dd) |
| 195 | δ = 8.36(2H, s), 8.19(2H, d), 8.18(1H, d), 8.03(1H, d), 7.98(1H, d), 7.82(1H, d), 7.79(3H, |
| | d), 7.74(1H, d), 7.62(2H, d), 7.58(1H, dd), 7.54(1H, d), 7.5(5H, d), 7.46(2H, dd), 7.41(1H, |
| | dd), 7.4(1H, s), 7.39 (1H, t), 7.31 (2H, t), 7.2 (1H, dd), 7.18 (1H, dd) |
| 198 | δ = 9.08(1H, s), 8.84(1H, d), 8.36(2H, d), 8.27(1H, d), 8.25(2H, d), 8.18(1H, d), 8.05(1H, |
| | d), 8.03(1H, d), 7.98(1H, d), 7.9(1H, dd), 7.82(1H, d), 7.79(2H, d), 7.7(1H, dd), 7.68(1H, |
| | dd), 7.64(1H, s), 7.63 (1H, t), 7.54 (1H, t), 7.5 (3H, dd), 7.46 (2H, dd), 7.39 (1H, t), 7.31 |
| | (2H, d), 7.25 (2H, d) |
| 205 | δ = 8.3(2H, s), 8.23(1H, d), 8.18(1H, d), 8.03(1H, d), 7.98(1H, d), 7.94(2H, d), 7.85(2H, |
| | d), 7.82(1H, d), 7.79(2H, d), 7.75(4H, dd), 7.71(1H, d), 7.61(1H, d), 7.54(1H, dd), |
| | 7.49(4H, dd), 7.46(2H, s), 7.41 (3H, t), 7.39 (1H, t), 7.31 (2H, dd) |
| 219 | δ = 8.54(1H, s), 8.18(1H, d), 8.13(1H, d), 8.03(1H, d), 7.99(1H, d), 7.98(1H, d), 7.83(1H, |
| | d), 7.82(1H, d), 7.8(2H, d), 7.79(2H, dd), 7.65(2H, d), 7.61(1H, d), 7.54(1H, dd), 7.53(1H, |
| | dd), 7.49(1H, s), 7.46 (2H, t), 7.41 (1H, t), 7.39 (1H, dd), 7.31 (2H, dd) |
| 220 | δ = 8.18(1H, s), 8.03(1H, d), 7.98(1H, d), 7.84(2H, d), 7.82(1H, d), 7.79(2H, d), 7.7(1H, |
| | d), 7.65(1H, d), 7.54(1H, d), 7.53(2H, dd), 7.49(1H, d), 7.46(2H, d), 7.41(1H, dd), |
| | 7.39(1H, dd), 7.36(1H, s), 7.31 (2H, t), 7.22 (1H, t) |
| 222 | δ = 8.54(1H, s), 8.18(1H, d), 8.03(1H, d), 7.99(1H, d), 7.98(1H, d), 7.84(2H, d), 7.82(1H, |
| | d), 7.79(2H, d), 7.61(1H, d), 7.54(1H, dd), 7.53(3H, d), 7.5(1H, d), 7.49(1H, dd), 7.46(2H, |
| | dd), 7.41(1H, s), 7.39 (1H, t), 7.31 (2H, t), 7.29 (1H, dd) |
| 228 | δ = 8.36(2H, s), 8.2(1H, d), 8.18(1H, d), 8.16(1H, d), 8.03(1H, d), 8.01(1H, d), 7.98(1H, |
| | d), 7.97(1H, d), 7.83(1H, d), 7.82(1H, dd), 7.79(2H, d), 7.78(1H, d), 7.67(1H, dd), |
| | 7.64(2H, dd), 7.54(1H, s), 7.5 (3H, t), 7.46 (2H, t), 7.41 (1H, dd), 7.39 (1H, dd), 7.31 (2H, |
| | t) |
| 230 | δ = 8.45(1H, s), 8.36(2H, d), 8.25(2H, d), 8.18(1H, d), 8.03(1H, d), 7.98(1H, d), 7.96(1H, |
| | d), 7.86(1H, d), 7.82(1H, d), 7.79(2H, dd), 7.67(1H, d), 7.56(1H, d), 7.54(1H, dd), 7.5(4H, |
| | dd), 7.46(2H, s), 7.41 (1H, t), 7.39 (1H, t), 7.31 (3H, dd), 7.25 (2H, dd) |
| 258 | δ = 8.25(2H, s), 8.18(1H, d), 8.03(1H, d), 7.98(2H, d), 7.94(1H, d), 7.82(1H, d), 7.75(4H, |
| | d), 7.71(1H, d), 7.61(2H, d), 7.6(1H, dd), 7.57(1H, d), 7.54(2H, d), 7.49(4H, dd), 7.47(1H, |
| | dd), 7.39(2H, s), 7.31 (3H, t), 7.25 (2H, t) |
| 280 | δ = 8.45(1H, s), 8.36(4H, d), 8.2(1H, d), 8.18(1H, d), 8.17(1H, d), 8.03(1H, d), 7.98(1H, |
| | d), 7.97(1H, d), 7.86(1H, d), 7.82(1H, dd), 7.56(1H, d), 7.54(1H, d), 7.5(6H, dd), 7.39(1H, |
| | dd), 7.31(3H, s) |
| 294 | δ = 9.07(1H, s), 8.36(4H, d), 8(1H, d), 7.98(1H, d), 7.82(1H, d), 7.75(2H, d), 7.54(1H, d), |
| | 7.5(6H, d), 7.49(2H, d), 7.41(1H, dd), 7.39(1H, d), 7.31(2H, d), 7.25(4H, dd) |
| 299 | δ = 9.07(1H, s), 8.23(1H, d), 8(1H, d), 7.98(1H, d), 7.94(4H, d), 7.82(1H, d), 7.79(2H, d), |
| | 7.75(2H, d), 7.71(1H, d), 7.61(1H, dd), 7.55(2H, d), 7.54(1H, d), 7.49(3H, dd), 7.46(2H, |
| | dd), 7.41(2H, s), 7.39 (1H, t), 7.31 (2H, t) |
| 301 | δ = 8.49(1H, s), 8.46(1H, d), 8.36(2H, d), 8.25(2H, d), 7.98(1H, d), 7.93(1H, d), 7.75(2H, |
| | d), 7.54(1H, d), 7.51(2H, d), 7.5(3H, dd), 7.49(2H, d), 7.46(2H, d), 7.41(2H, dd), 7.39(2H, |
| | dd), 7.31(1H, s), 7.25 (2H, t) |
| 304 | δ = 8.49(1H, s), 8.46(1H, d), 8.36(2H, d), 8.25(2H, d), 7.94(1H, d), 7.93(1H, d), 7.83(1H, |
| | d), 7.75(4H, d), 7.71(1H, d), 7.69(1H, dd), 7.63(1H, d), 7.61(2H, d), 7.5(4H, dd), 7.49(4H, |
| | dd), 7.41(2H, s), 7.29 (1H, t), 7.25 (2H, t), |
| 307 | δ = 9.09(1H, s), 8.49(2H, d), 8.46(1H, d), 8.36(2H, d), 8.16(1H, d), 8.09(1H, d), 8.08(1H, |
| | d), 8.06(1H, d), 8(1H, d), 7.99(1H, dd), 7.93(1H, d), 7.78(1H, d), 7.76(1H, dd), 7.63(1H, |
| | dd), 7.61(1H, s), 7.58 (1H, t), 7.57 (2H, t), 7.55 (1H, dd), 7.5 (4H, dd), 7.38 (1H, t), 7.29 |
| | (1H, d) |
| 319 | δ = 9.09(1H, s), 8.49(2H, d), 8.46(1H, d), 8.16(1H, d), 8.09(1H, d), 8.08(1H, d), 8.06(1H, |
| | d), 8(1H, d), 7.99(1H, d), 7.98(1H, dd), 7.93(1H, d), 7.78(1H, d), 7.76(1H, dd), 7.63(1H, |
| | dd), 7.61(1H, s), 7.6 (1H, t), 7.58 (1H, t), 7.57 (3H, dd), 7.55 (1H, dd), 7.54 (1H, t), 7.5 (1H, |
| | d), 7.47 (1H, d), 7.39 (1H, d) |
| 331 | δ = 8.49(1H, s), 8.46(1H, d), 8.36(4H, d), 8.09(1H, d), 8.06(1H, d), 7.99(1H, d), 7.93(1H, |
| | d), 7.78(1H, d), 7.76(1H, d), 7.63(1H, dd), 7.58(1H, d), 7.57(1H, d), 7.55(1H, dd), 7.5(7H, |
| | dd), 7.38(1H, s), 7.29 (1H, t), 7.25 (4H, t) |
| 346 | δ = 9.09(1H, s), 8.49(1H, d), 8.36(2H, d), 8.09(1H, d), 8.08(1H, d), 8.06(1H, d), 8.02(1H, |
| | d), 7.99(1H, d), 7.98(2H, d), 7.63(1H, dd), 7.6(1H, d), 7.58(1H, d), 7.57(1H, dd), 7.55(1H, |
| | dd), 7.54(1H, s), 7.5 (4H, t), 7.47 (1H, t), 7.41 (1H, dd), 7.39 (1H, dd), 7.38 (1H, t), 7.31 |
| | (1H, d), 7.29 (1H, d) |
| 371 | δ = 9.09(2H, s), 8.49(2H, d), 8.25(2H, d), 8.16(1H, d), 8.08(1H, d), 8(1H, d), 7.98(2H, d), |
| | 7.75(2H, d), 7.61(1H, d), 7.57(1H, dd), 7.54(1H, d), 7.51(2H, d), 7.49(2H, dd), 7.46(2H, |
| | dd), 7.41(2H, s), 7.39 (2H, t), 7.31 (1H, t), 7.25 (2H, dd) |
| 377 | δ = 9.09(1H, s), 9.07(1H, d), 8.49(1H, d), 8.36(2H, d), 8.16(1H, d), 8.08(1H, d), 8(2H, d), |
| | 7.98(1H, d), 7.82(1H, d), 7.79(2H, dd), 7.61(1H, d), 7.57(1H, d), 7.54(1H, dd), 7.5(3H, |
| | dd), 7.46(2H, s), 7.41 (1H, t), 7.39 (1H, t), 7.31 (2H, dd) |
| 387 | δ = 9.07(1H, s), 8.55(1H, d), 8.36(2H, d), 8(1H, d), 7.82(1H, d), 7.78(1H, d), 7.76(1H, d), |
| | 7.75(2H, d), 7.62(2H, d), 7.58(1H, dd), 7.57(1H, d), 7.52(1H, d), 7.5(7H, dd), 7.49(2H, |
| | dd), 7.42(1H, s), 7.41 (1H, t), 7.37 (1H, t), 7.29 (1H, dd), 7.16 (1H, dd), 7.11 (1H, t) |
| A1 | δ = 8.55 (1H, s), 8.18 (1H, d), 7.79 (2H, d), 7.71 (1H, d), 7.65 (1H, d), 7.55 (2H, d), 7.54 |
| | (4H, d), 7.52 (4H, d), 7.51 (6H, d), 7.41 (4H, dd), 6.69 (4H, d), 6.39 (1H, d) |
| A3 | δ = 8.93 (1H, s), 8.68 (1H, d), 8.55 (1H, d), 8.18 (1H, d), 8.12 (1H, d), 7.88 (1H, d), 7.82 |
| | (1H, d), 7.79 (2H, d), 7.71 (3H, d), 7.65 (1H, dd), 7.55 (2H, d), 7.54 (2H, d), 7.52 (2H, dd), |
| | 7.51 (4H, dd), 7.41 (3H, s), 7.32 (1H, t), 7.08 (1H, t), 6.69 (2H, dd), 6.39 (1H, dd) |
| A4 | δ = 8.55 (1H, s), 8.18 (1H, d), 7.81 (1H, d), 7.79 (2H, d), 7.72 (1H, d), 7.71 (2H, d), 7.55 |
| | (2H, d), 7.54 (6H, d), 7.52 (4H, d), 7.51 (6H, dd), 7.41 (3H, d), 6.69 (6H, d) |
| A7 | δ = 8.55 (1H, s), 8.18 (1H, d), 7.89 (1H, d), 7.81 (1H, d), 7.79 (2H, d), 7.72 (1H, d), 7.71 |
| | (2H, d), 7.66 (1H, d), 7.64 (1H, d), 7.55 (2H, dd), 7.54 (4H, d), 7.52 (2H, d), 7.51 (4H, dd), |
| | 7.43 (1H, dd), 7.41 (2H, s), 7.38 (1H, t), 7.32 (1H, t), 6.69 (4H, dd), 6.33 (1H, dd) |
| A11 | δ = 8.55 (1H, s), 8.18 (1H, d), 7.88 (1H, d), 7.84 (1H, d), 7.79 (2H, d), 7.77 (1H, d), 7.74 |
| | (1H, d), 7.71 (1H, d), 7.55 (2H, d), 7.54 (2H, dd), 7.52 (2H, d), 7.51 (4H, d), 7.5 (1H, dd), |
| | 7.49 (1H, dd), 7.41 (2H, s), 7.36 (1H, t), 7.25 (1H, t), 7.07 (1H, dd), 6.69 (2H, dd), 6.39 |
| | (1H, t) |
| A12 | δ = 8.55 (1H, s), 8.18 (1H, d), 7.85 (1H, d), 7.81 (1H, d), 7.79 (2H, d), 7.71 (1H, d), 7.55 |
| | (2H, d), 7.54 (5H, d), 7.52 (2H, d), 7.51 (6H, dd), 7.41 (3H, d), 7.38 (1H, d), 7.16 (1H, dd), |
| | 7.08 (2H, dd), 6.87 (1H, s), 6.69 (5H, t) |
| A13 | δ = 8.55 (1H, s), 8.18 (1H, d), 8 (2H, d), 7.95 (1H, d), 7.92 (1H, d), 7.75 (1H, d), 7.73 (1H, |
| | d), 7.71 (1H, d), 7.64 (1H, d), 7.59 (2H, dd), 7.58 (1H, d), 7.55 (2H, d), 7.54 (5H, dd), 7.52 |
| | (2H, dd), 7.51 (4H, s), 7.41 (2H, t), 7.16 (1H, t), 7.08 (2H, dd), 6.87 (1H, dd), 6.69 (5H, t) |
| A16 | δ = 8.55 (1H, s), 8.18 (1H, d), 7.88 (1H, d), 7.84 (1H, d), 7.79 (2H, d), 7.77 (1H, d), 7.74 |
| | (1H, d), 7.71 (1H, d), 7.55 (2H, d), 7.54 (2H, dd), 7.52 (2H, d), 7.51 (4H, d), 7.5 (1H, dd), |
| | 7.49 (1H, dd), 7.41 (2H, s), 7.36 (1H, t), 7.25 (4H, t), 7.13 (1H, dd), 7.02 (1H, dd), 6.69 |
| | (2H, t), 6.33 (1H, d) |
| A17 | δ = 8.55 (1H, s), 8.18 (1H, d), 7.79 (2H, d), 7.75 (1H, d), 7.71 (1H, d), 7.62 (1H, d), 7.55 |
| | (2H, d), 7.54 (4H, d), 7.52 (4H, d), 7.51 (6H, dd), 7.44 (2H, d), 7.41 (3H, d), 7.25 (4H, dd), |
| | 6.89 (1H, dd), 6.88 (1H, s), 6.69 (4H, t), 6.59 (1H, t) |
| A20 | δ = 8.55 (1H, s), 8.18 (1H, d), 7.79 (2H, d), 7.75 (1H, d), 7.71 (1H, d), 7.62 (1H, d), 7.55 |
| | (2H, d), 7.54 (6H, d), 7.52 (4H, d), 7.51 (6H, dd), 7.44 (1H, d), 7.41 (3H, d), 6.69 (6H, dd) |
| A21 | δ = 8.93 (2H, s), 8.55 (1H, d), 8.18 (1H, d), 8.12 (2H, d), 7.93 (1H, d), 7.88 (2H, d), 7.82 |
| | (2H, d), 7.79 (2H, d), 7.71 (1H, d), 7.55 (2H, dd), 7.54 (4H, d), 7.52 (2H, d), 7.51 (4H, dd), |
| | 7.41 (2H, dd), 7.13 (1H, s), 7.02 (1H, t), 6.69 (4H, t), 6.33 (1H, dd) |
| A24 | δ = 8.55 (1H, s), 8.18 (1H, d), 7.89 (1H, d), 7.79 (2H, d), 7.75 (1H, d), 7.71 (1H, d), 7.66 |
| | (1H, d), 7.64 (1H, d), 7.62 (1H, d), 7.55 (2H, dd), 7.54 (4H, d), 7.52 (2H, d), 7.51 (4H, dd), |
| | 7.44 (1H, dd), 7.43 (1H, s), 7.41 (2H, t), 7.38 (1H, t), 7.32 (1H, dd), 6.69 (4H, dd), 6.33 |
| | (1H, t) |
| A25 | δ = 8.55 (1H, s), 8.18 (1H, d), 7.95 (1H, d), 7.79 (2H, d), 7.75 (1H, d), 7.71 (1H, d), 7.64 |
| | (1H, d), 7.55 (2H, d), 7.54 (6H, d), 7.52 (4H, dd), 7.51 (6H, d), 7.41 (3H, d), 6.69 (6H, dd) |
| A26 | δ = 8.55 (1H, s), 8.18 (1H, d), 7.79 (2H, d), 7.71 (1H, d), 7.64 (1H, d), 7.55 (2H, d), 7.54 |
| | (4H, d), 7.52 (4H, d), 7.51 (6H, d), 7.43 (1H, dd), 7.41 (3H, d), 6.69 (4H, d), 6.33 (1H, dd) |
| A29 | δ = 8.93 (2H, s), 8.55 (1H, d), 8.18 (1H, d), 8.12 (2H, d), 7.93 (1H, d), 7.88 (2H, d), 7.82 |
| | (2H, d), 7.79 (2H, d), 7.71 (1H, d), 7.64 (1H, dd), 7.55 (2H, d), 7.54 (2H, d), 7.51 (2H, dd), |
| | 7.43 (1H, dd), 7.41 (1H, s), 7.2 (2H, t), 6.81 (1H, t), 6.69 (2H, dd), 6.63 (2H, dd), 6.33 (1H, |
| | t) |
| A37 | δ = 7.52 (2H, s), 7.51 (2H, d), 7.41 (1H, d) |
| A41 | δ = 8.55 (2H, s), 7.79 (2H, d), 7.64 (2H, d), 7.55 (2H, d), 7.54 (4H, d), 7.52 (4H, d), 7.51 |
| | (6H, d), 7.43 (1H, d), 7.41 (3H, d), 6.69 (4H, dd), 6.33 (1H, d) |
| A58 | δ = 8.55 (2H, s), 7.95 (1H, d), 7.79 (2H, d), 7.75 (1H, d), 7.64 (2H, d), 7.55 (2H, d), 7.54 |
| | (2H, d), 7.52 (2H, d), 7.51 (4H, d), 7.44 (1H, dd), 7.41 (2H, d), 7.2 (2H, d), 6.89 (1H, dd), |
| | 6.88 (1H, dd), 6.81 (1H, s), 6.69 (2H, t), 6.63 (2H, t), 6.59 (1H, dd) |
| A66 | δ = 8.55 (2H, s), 7.79 (2H, d), 7.64 (1H, d), 7.55 (2H, d), 7.54 (2H, d), 7.52 (2H, d), 7.51 |
| | (4H, d), 7.41 (2H, d), 7.25 (5H, d), 7.2 (2H, dd), 7.07 (1H, d), 6.81 (1H, d), 6.69 (2H, dd), |
| | 6.63 (2H, dd), 6.39 (1H, s) |
| A69 | δ = 8.55 (2H, s), 7.88 (1H, d), 7.84 (1H, d), 7.79 (2H, d), 7.77 (1H, d), 7.74 (1H, d), 7.65 |
| | (1H, d), 7.64 (1H, d), 7.55 (2H, d), 7.54 (2H, dd), 7.52 (2H, d), 7.51 (4H, d), 7.5 (1H, dd), |
| | 7.49 (1H, dd), 7.41 (3H, s), 7.36 (1H, t), 6.69 (2H, t), 6.39 (1H, dd) |
| A74 | δ = 8.55 (2H, s), 7.79 (2H, d), 7.64 (1H, d), 7.55 (2H, d), 7.54 (2H, d), 7.52 (2H, d), 7.51 |
| | (4H, d), 7.41 (2H, d), 7.25 (4H, d), 7.2 (2H, dd), 7.13 (1H, d), 7.02 (1H, d), 6.81 (1H, dd), |
| | 6.69 (2H, dd), 6.63 (2H, s), 6.33 (1H, t) |
| A78 | δ = 8.55 (2H, s), 7.81 (1H, d), 7.79 (2H, d), 7.72 (1H, d), 7.71 (1H, d), 7.64 (1H, d), 7.55 |
| | (2H, d), 7.54 (4H, d), 7.52 (2H, d), 7.51 (4H, dd), 7.41 (2H, d), 7.2 (2H, d), 6.81 (1H, dd), |
| | 6.69 (4H, dd), 6.63 (2H, s) |
| A81 | δ = 8.16 (2H, s), 7.79 (2H, d), 7.67 (2H, d), 7.54 (4H, d), 7.52 (4H, d), 7.51 (7H, d), 7.49 |
| | (1H, d), 7.42 (1H, d), 7.41 (3H, d), 7.39 (1H, dd), 6.69 (4H, d) |
| A86 | δ = 8.16 (2H, s), 7.79 (2H, d), 7.67 (2H, d), 7.6 (1H, d), 7.57 (1H, d), 7.54 (4H, d), 7.52 |
| | (2H, d), 7.51 (4H, d), 7.49 (1H, d), 7.42 (1H, dd), 7.41 (2H, d), 7.2 (2H, d), 6.81 (1H, dd), |
| | 6.69 (4H, dd), 6.63 (2H, s) |
| A99 | δ = 8.16 (2H, s), 7.67 (2H, d), 7.6 (1H, d), 7.54 (2H, d), 7.52 (4H, d), 7.51 (4H, d), 7.49 |
| | (1H, d), 7.42 (1H, d), 7.41 (2H, d), 7.25 (4H, dd), 7.2 (2H, d), 6.81 (1H, d), 6.69 (2H, dd), |
| | 6.63 (2H, dd), 6.39 (1H, s) |

TABLE 5

| Compound | | Compound | |
|---|---|---|---|
| number | FD-MS | number | FD-MS |
| 7 | m/z = 677.25 (C49H31N3O, | 198 | m/z = 701.25 (C51H31N3O, |
| | 677.81) | | 701.83) |
| 9 | m/z = 575.20 (C41H25N3O, | 205 | m/z = 676.25 (C50H32N2O, |
| | 575.67) | | 676.82) |
| 20 | m/z = 651.23 (C47H29N3O, | 219 | m/z = 548.19 (C40H24N2O, |
| | 651.77) | | 548.65) |
| 21 | m/z = 615.19 (C43H25N3O2, | 220 | m/z = 538.17 (C38H22N2O2, |
| | 615.69) | | 538.61) |
| 27 | m/z = 665.21 (C47H27N3O2, | 222 | m/z = 588.18(C42H24N202, |
| | 665.75) | | 588.67) |
| 30 | m/z = 705.21 (C49H27N3O3, | 228 | m/z = 681.19 (C47H27N3OS, |
| | 705.77) | | 681.81) |
| 35 | m/z = 631.17 (C43H25N3OS, | 230 | m/z = 707.20 (C49H29N3OS, |
| | 631.75) | | 707.85) |
| 44 | m/z = 690.24 (C49H30N4O, | 258 | m/z = 767.26 (C55H33N3O2, |
| | 690.81) | | 767.89) |
| 47 | m/z = 701.25 (C51H31N3O, | 280 | m/z = 631.17 (C43H25N3OS, |
| | 701.83) | | 631.75) |
| 50 | m/z = 600.22 (C44H28N2O, | 294 | m/z = 601.22 (C43H27N3O, |
| | 600.72) | | 601.71) |
| 61 | m/z = 574.20 (C42H26N2O, | 299 | m/z = 600.22 (C44H28N2O, |
| | 574.68) | | 600.72) |
| 65 | m/z = 498.17 (C36H22N2O, | 301 | m/z = 601.22 (C43H27N3O, |
| | 498.59) | | 601.71) |
| 70 | m/z = 588.18 = | 304 | m/z = 677.25 (C49H31N3O, |
| | (C42H24N2O2, 588.67) | | 677.81) |
| 74 | m/z = 665.21 | 307 | m/z = 625.22 (C45H27N3O, |
| | (C47H27N3O2, 665.75) | | 625.73) |
| 77 | m/z = 691.23 (C49H29N3O2, | 319 | m/z = 715.23 (C51H29N3O2, |
| | 691.79) | | 715.81) |
| 81 | m/z = 651.23 (C47H29N3O, | 331 | m/z = 651.23 (C47H29N3O, |
| | 651.77) | | 651.77) |
| 99 | m/z = 701.25 (C51H31N3O, | 346 | m/z = 665.21 (C47H27N3O2, |
| | 701.83) | | 665.75) |
| 127 | m/z = 665.21 (C47H27N3O2, | 371 | m/z = 651.23 (C47H29N3O, |
| | 665.75) | | 651.77) |
| 137 | m/z = 677.25 (C49H31N3O, | 377 | m/z = 575.20 (C41H25N3O, |
| | 677.81) | | 575.67) |
| 175 | m/z = 615.19 (C43H25N3O2, | 387 | m/z = 690.24 (C49H30N4O, |
| | 615.69) | | 690.81) |
| 177 | m/z = 691.23 (C49H29N3O2, | 390 | m/z = 675.27 (C47H17D10N3O2, |
| | 691.79) | | 675.81) |
| 195 | m/z = 690.24 (C49H30N4O, | 396 | m/z = 621.34 (C43H7D20N3O, |
| | 690.81) | | 621.83) |
| A1 | m/z = 613.24 (C46H31NO, | A37 | m/z = 639.40 (C46H5D26NO, |
| | 613.74) | | 639.90) |
| A3 | m/z = 637.24 (C48H31NO, | A41 | m/z = 613.24 (C46H31NO, |
| | 637.77) | | 613.74) |
| A4 | m/z = 689.27 | A58 | m/z = 613.24 (C46H31NO, |
| | (C52H35NO, 689.84) | | 613.74) |
| A7 | m/z = 703.25 | A66 | m/z = 613.24 (C46H31NO, |
| | (C52H33NO2, 703.82) | | 613.74) |
| A11 | m/z = 587.22 (C44H29NO, | A69 | m/z = 587.22 (C44H29NO, |
| | 587.71) | | 587.71) |
| A12 | m/z = 689.27 (C52H35NO, | A74 | m/z = 613.24 (C46H31NO, |
| | 689.84) | | 613.74) |
| A13 | m/z = 739.29 (C56H37NO, | A78 | m/z = 613.24 (C46H31NO, |
| | 739.90) | | 613.74) |
| A16 | m/z = 663.26 (C50H33NO, | A88 | m/z = 663.26 (C50H33NO, |
| | 663.80) | | 663.80) |
| A17 | m/z = 765.30 (C58H39NO, | A91 | m/z = 637.24 (C48H31NO, |
| | 765.94) | | 637.77) |
| A20 | m/z = 689.27 (C52H35NO, | A93 | m/z = 703.25 (C52H33NO2, |
| | 689.84) | | 703.82) |
| A21 | m/z = 713.27 (C54H35NO, | A98 | m/z = 634.37 (C46H10D21NO, |
| | 713.86) | | 634.87) |
| A24 | m/z = 703.25 (C52H33NO2, | A81 | m/z = 613.24 (C46H31NO, |
| | 703.82) | | 613.74) |
| A25 | m/z = 689.27 (C52H35NO, | A86 | m/z = 613.24 (C46H31NO, |
| | 689.84) | | 613.74) |
| A26 | m/z = 613.24 (C46H31NO, | A99 | m/z = 613.24 (C46H31NO, |
| | 613.74) | | 613.74) |
| A29 | m/z = 637.24 (C48H31NO, | | |
| | 637.77) | | |

### Manufacture of Organic Light-Emitting Device 1

A glass substrate coated with an indium tin oxide (ITO) AgTTO thin film having a thicknesses of 115 Å/100 Å/15 Å was ultrasonically washed in distilled water. After completion of distilled water washing the substrate was ultrasonicaly washed with solvents such as acetone, methanol, and isopropyl alcohol. After drying, the substrate was subjected to ultraviolet ozone (UVO) treatment for 5 minutes in an ultraviolet (UV) cleaner. The substrate was then transferred to a plasma cleaner (PC) and subjected to plasma treatment under vacuum to adjust the ITO work function and remove any residual film. The substrate was then transferred to a thermal evaporation device for organic vapor deposition. A common layer of a hole injection layer, HAT-CN 100 Å, a hole transport layer α-NPB 1100 Å, a photo-assisting layer TPD (N-([1,1′-biphenyl]-4-yl)-N,1′-diphenyl-1′H-spiro[fluorene-9,5′-naphtho[8,1,2,3-cdef]carbazol]-7-amine) 800 Å, and an electron blocking layer TAPC (N-([1,1′-biphenyl]-4-yl)-N,1′-diphenyl-1′H-spiro[fluorene-9,5′-naphtho[8,1,2,3-cdef]carbazol]-7-amine) 150 Å, were formed on the ITO electrode (anode). A light-emitting layer was thermally vacuum-deposited on the electron blocking layer as follows.

The light-emitting layer was deposited using either a single compound listed in Table 6 below as a red host or two compounds listed in Table 7 below co-deposited from a single source. The host was doped with 3% by weight of (piq)₂Ir(acac) as a red phosphorescent dopant, and the light-emitting layer was deposited in a thickness of 400 Å.

Subsequently, Bphen was deposited on the light-emitting layer in a thickness of 30 Å as a hole-blocking layer, and TPBI was deposited thereon in a thickness of 250 Å as an electron transport layer. Finally, lithium fluoride (LiF) was deposited on the electron transport layer in a thickness of 10 Å to form an electron injection layer. Then, a silver (Ag) cathode was deposited in a thickness of 200 Å on the electron injection layer to form a cathode, thereby manufacturing an organic electroluminescent device. Meanwhile, all organic compounds required for the manufacture of the light-emitting device were purified by vacuum sublimation at 10-⁸ to 10-6 torr for each material and used in the manufacture of the light-emitting device.

Here, the comparative compounds A to O used in the comparative examples are as follows.

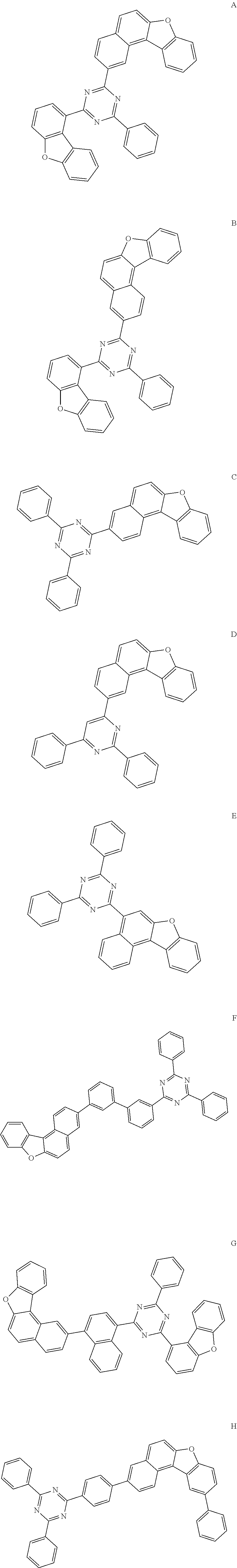

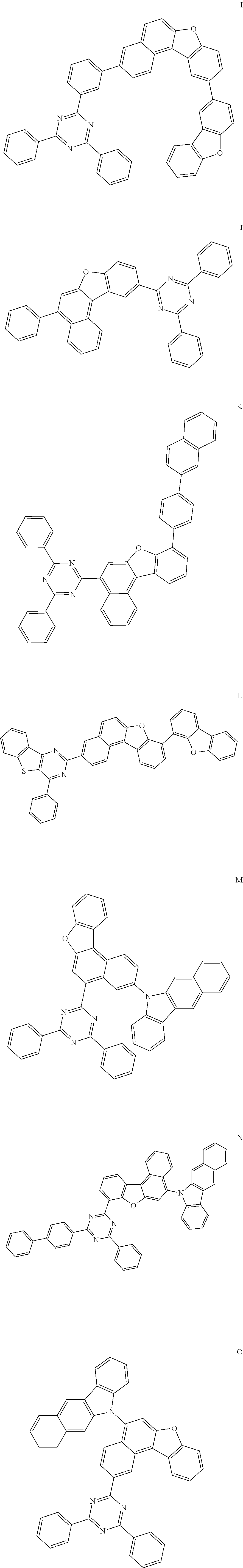

The electroluminescence (EL) characteristics of the organic electroluminescent devices manufactured as described above were measured using the M7000 system from MacScience. Based on the measurement results, the lifespan (T95) was measured using a lifespan measurement system (M6000) manufactured by MacScience at a reference luminance of 6,000 cd/m2. T95 represents the lifespan (unit: h (hours)), which is the time required for the initial luminance to decrease to 95% of its original value.

Table 6 below shows examples employing a single host material, and Table 7 shows examples in which two host compounds were co-deposited from a single source, using a compound of the present invention having excellent electron-transport capability (acceptor, n-Host) as the first host and a compound having excellent hole-transport capability (donor, p-Host) as the second host.

TABLE 6

| | | Operating | Efficiency | Lifetime |
|---|---|---|---|---|
| | Compound | voltage (V) | (cd/A) | (T95) |
| Comparative | A | 4.92 | 10.5 | 17 |
| Example 1 | | | | |
| Comparative | B | 4.96 | 10.2 | 15 |
| Example 2 | | | | |
| Comparative | C | 4.97 | 9.9 | 14 |
| Example 3 | | | | |
| Comparative | D | 4.94 | 9.2 | 11 |
| Example 4 | | | | |
| Comparative | E | 5.08 | 7.7 | 8 |
| Example 5 | | | | |
| Comparative | F | 5.13 | 6.4 | 9 |
| Example 6 | | | | |
| Comparative | G | 5.11 | 6.8 | 10 |
| Example 7 | | | | |
| Comparative | H | 5.03 | 7.2 | 11 |
| Example 8 | | | | |
| Comparative | I | 5.05 | 7 | 12 |
| Example 9 | | | | |
| Comparative | J | 5.03 | 8.8 | 8 |
| Example 10 | | | | |
| Comparative | K | 5.07 | 8.4 | 9 |
| Example 11 | | | | |
| Comparative | L | 4.98 | 9.3 | 7 |
| Example 12 | | | | |
| Comparative | M | 5.1 | 5.8 | 8 |
| Example 13 | | | | |
| Comparative | N | 5.04 | 5.5 | 10 |
| Example 14 | | | | |
| Comparative | O | 5.01 | 6.8 | 12 |
| Example 15 | | | | |
| Example 1 | 7 | 3.75 | 30.1 | 38 |
| Example 2 | 9 | 3.77 | 30.6 | 44 |
| Example 3 | 20 | 3.79 | 30.7 | 42 |
| Example 4 | 21 | 3.74 | 30.9 | 40 |
| Example 5 | 27 | 3.72 | 31.6 | 48 |
| Example 6 | 30 | 3.75 | 30.7 | 46 |
| Example 7 | 35 | 3.77 | 28.4 | 35 |
| Example 8 | 44 | 3.8 | 28.2 | 38 |
| Example 9 | 47 | 3.84 | 26.7 | 31 |
| Example 10 | 50 | 3.83 | 26.7 | 31 |
| Example 11 | 61 | 3.84 | 27.2 | 29 |
| Example 12 | 65 | 3.82 | 27 | 27 |
| Example 13 | 70 | 3.99 | 21.8 | 25 |
| Example 14 | 74 | 3.92 | 28.1 | 30 |
| Example 15 | 77 | 3.86 | 30.1 | 39 |
| Example 16 | 81 | 3.74 | 31.2 | 39 |
| Example 17 | 99 | 3.74 | 31.8 | 46 |
| Example 18 | 127 | 3.81 | 30.9 | 42 |
| Example 19 | 137 | 3.89 | 20.7 | 33 |
| Example 20 | 175 | 3.88 | 21.2 | 30 |
| Example 21 | 177 | 3.86 | 21.4 | 32 |
| Example 22 | 195 | 3.92 | 21 | 29 |
| Example 23 | 198 | 3.95 | 24.7 | 20 |
| Example 24 | 205 | 4.15 | 20.8 | 24 |
| Example 25 | 219 | 4.19 | 19.2 | 19 |
| Example 26 | 220 | 4.13 | 21.2 | 25 |
| Example 27 | 222 | 4.17 | 20.2 | 22 |
| Example 28 | 228 | 4.05 | 19.8 | 18 |
| Example 29 | 230 | 4.08 | 20.6 | 24 |
| Example 30 | 258 | 3.95 | 21.8 | 32 |
| Example 31 | 280 | 4.06 | 20.8 | 26 |
| Example 32 | 294 | 4.12 | 23.1 | 30 |
| Example 33 | 299 | 4.08 | 22.6 | 26 |
| Example 34 | 301 | 3.72 | 29.6 | 35 |
| Example 35 | 304 | 3.84 | 25.7 | 30 |
| Example 36 | 307 | 3.75 | 31.9 | 42 |
| Example 37 | 319 | 3.71 | 32.4 | 46 |
| Example 38 | 331 | 3.72 | 32 | 45 |
| Example 39 | 346 | 3.84 | 21.5 | 29 |
| Example 40 | 371 | 3.85 | 20.1 | 30 |
| Example 41 | 377 | 3.94 | 23.6 | 32 |
| Example 42 | 387 | 3.9 | 24.4 | 28 |
| Example 43 | 390 | 3.8 | 22.5 | 43 |
| Example 44 | 396 | 3.75 | 30.2 | 57 |

TABLE 7

| | | | | Operating | | |
|---|---|---|---|---|---|---|
| | First | Second | Ratio | voltage | Efficiency | Lifetime |
| | host | host | (N:P) | (V) | (cd/A) | (T95) |
| Comparative | A1 | A | 1:1 | 3.8 | 19.7 | 35 |
| Example 16 | | | | | | |
| Example 45 | | 21 | 1:1 | 3.05 | 63.3 | 90 |
| Comparative | A3 | B | 1:1 | 3.84 | 19.2 | 31 |
| Example 17 | | | | | | |
| Example 46 | | 177 | 1:1 | 3.17 | 43.9 | 72 |
| Comparative | A11 | C | 1:1 | 3.85 | 18.6 | 29 |
| Example 18 | | | | | | |
| Example 47 | | 137 | 1:1 | 3.2 | 42.4 | 74 |
| Comparative | A16 | D | 1:1 | 3.82 | 17.3 | 23 |
| Example 19 | | | | | | |
| Example 48 | | 50 | 1:1 | 3.14 | 54.7 | 70 |
| Comparative | A21 | E | 1:1 | 3.96 | 14.5 | 17 |
| Example 20 | | | | | | |
| Comparative | | K | 1:1 | 3.95 | 15.8 | 19 |
| Example 21 | | | | | | |
| Example 49 | | 377 | 1:1 | 3.25 | 48.4 | 72 |
| Comparative | A29 | F | 1:1 | 4.01 | 11.9 | 19 |
| Example 22 | | | | | | |
| Example 50 | | 371 | 1:1 | 3.16 | 41.0 | 67 |
| Comparative | | G | 1:1 | 3.99 | 12.6 | 21 |
| Example 23 | | | | | | |
| Example 51 | | 21 | 1:1 | 3.05 | 63.0 | 89 |
| Comparative | A69 | H | 1:1 | 3.91 | 13.5 | 23 |
| Example 24 | | | | | | |
| Example 52 | | 304 | 1:1 | 3.15 | 52.7 | 67 |
| Comparative | | I | 1:1 | 3.93 | 13.2 | 25 |
| Example 25 | | | | | | |
| Example 53 | | 280 | 1:1 | 3.37 | 42.6 | 58 |
| Comparative | A66 | L | 1:1 | 3.86 | 17.5 | 15 |
| Example 26 | | | | | | |
| Example 54 | | 220 | 1:1 | 3.44 | 43.5 | 56 |
| Comparative | A74 | M | 1:1 | 3.98 | 10.9 | 17 |
| Example 27 | | | | | | |
| Example 55 | | 195 | 1:1 | 3.23 | 43.1 | 65 |
| Comparative | | N | 1:1 | 3.92 | 10.3 | 21 |
| Example 28 | | | | | | |
| Example 56 | | 387 | 1:1 | 3.21 | 50.0 | 63 |
| Comparative | A99 | O | 1:1 | 3.89 | 12.8 | 25 |
| Example 29 | | | | | | |
| Example 57 | | 44 | 1:1 | 3.11 | 57.8 | 85 |
| Example 58 | A41 | 7 | 1:1 | 3.06 | 61.7 | 85 |
| Example 59 | | 9 | 1:1 | 3.08 | 62.7 | 99 |
| Example 60 | | 20 | 1:1 | 3.1 | 62.9 | 94 |
| Example 61 | A81 | 27 | 1:1 | 3.03 | 64.8 | 108 |
| Example 62 | | 30 | 1:1 | 3.06 | 62.9 | 103 |
| Example 63 | | 35 | 1:1 | 3.08 | 58.2 | 79 |
| Example 64 | | 61 | 1:1 | 3.15 | 55.8 | 65 |
| Example 65 | | 77 | 1:1 | 3.17 | 61.7 | 88 |
| Example 66 | A1 | 81 | 1:1 | 3.05 | 64.0 | 88 |
| Example 67 | | 99 | 1:1 | 3.05 | 65.2 | 103 |
| Example 68 | | 127 | 1:1 | 3.12 | 63.3 | 94 |
| Example 69 | | 175 | 1:1 | 3.19 | 43.5 | 67 |
| Example 70 | | 205 | 1:1 | 3.46 | 42.6 | 54 |
| Example 71 | A11 | 230 | 1:1 | 3.39 | 42.2 | 54 |
| Example 72 | | 258 | 1:1 | 3.26 | 44.7 | 72 |
| Example 73 | | 294 | 1:1 | 3.43 | 47.4 | 67 |
| Example 74 | | 299 | 1:1 | 3.39 | 46.3 | 58 |
| Example 75 | | 301 | 1:1 | 3.03 | 60.7 | 79 |
| Example 76 | | 307 | 1:1 | 3.06 | 65.4 | 94 |
| Example 77 | A74 | 319 | 1:1 | 3.02 | 66.4 | 103 |
| Example 78 | | 331 | 1:1 | 3.03 | 65.6 | 101 |
| Example 79 | | 346 | 1:1 | 3.15 | 44.1 | 65 |
| Example 80 | | 390 | 1:1 | 3.11 | 46.1 | 120 |
| Example 81 | | 396 | 1:1 | 3.06 | 61.9 | 160 |

As can be seen from Tables 6 and 7, the heterocyclic compounds of the present invention exhibit lower operating voltages and significantly improved light-emitting efficiency and lifetime compared to the comparative compounds. The heterocyclic compounds of the present invention exhibit excellent thermal stability and possess molecular weights and band gaps suitable for use in the light-emitting layer of organic light-emitting devices. The appropriate molecular weight facilitates the formation of the light-emitting layer in the organic light-emitting device, and the appropriate band gap prevents the loss of electrons and holes in the light-emitting layer, thereby facilitating the formation of an effective recombination region.

The light-emitting devices of the examples employing a single host exhibited T95 lifetime values of 24 hours or more, operating efficiencies of 19.2 cd/A or higher, and operating voltages of 4.15 V or less. In contrast, the light-emitting devices of the comparative examples employing a single host exhibited T95 lifetime values of 17 hours or less, operating efficiencies of 10.5 cd/A or lower, and operating voltages of 4.92 V or higher.

The light-emitting devices of the examples employing two hosts exhibited T95 lifetime values of 54 hours or more, operating voltages of 3.46 V or less, and operating efficiencies of 42.2 cd/A or higher. In contrast, the light-emitting devices of the comparative examples employing two hosts exhibited T95 lifetime values of 35 hours or less, operating voltages of 3.8 V or higher, and operating efficiencies of 19.7 cd/A or lower.

Compounds A to G of the comparative examples exhibited significantly higher operating voltages and lower efficiency and lifetime compared to the heterocyclic compounds of the present invention. These results demonstrate that the compounds of the present invention, with their aryl group and electron-transporting azine substituents substituted onto the naphthobenzofuran parent nucleus, spatially separate the Highest Occupied Molecular Orbital (HOMO) and Lowest Unoccupied Molecular Orbital (LUMO), thereby enabling strong charge transfer. In contrast, the comparative compounds, in which the parent nucleus is monosubstituted with only a simple azine substituent and does not contain an aryl substituent, fail to achieve separation between the HOMO and the LUMO, thereby hindering charge transfer.

Compounds F to I of the comparative examples do not directly bond the azine substituent to the naphthobenzofuran parent nucleus, but rather are connected via a phenylene or naphthalene linker. This structure exhibits a high operating voltage because the rotation between the naphthobenzofuran parent nucleus and the azine substituents increases, leading to a distortion of the conjugation.

Compounds J and K of the comparative examples exhibited significantly increased operating voltage and lower efficiency compared to the heterocyclic compound of the present invention. These results are because, unlike Formula 1 of the present disclosure, where the azine substituents are substituted onto the naphthalenes of R¹ to R₄, thereby expanding the conjugation, the azine substituents are substituted at different positions, thereby disrupting the conjugation and excessively increasing the band gap, resulting in reduced efficiency.

Compound L of the comparative examples is a thieno-pyrimidine-substituted form. Compared to the benzofuropyrimidine compound 220 of the present invention, the presence of sulfur, which is larger than oxygen, results in weaker bonding energy with pyrimidine, thereby leading to significantly lower thermal stability and decreased lifetime.

Compounds M to O of the comparative examples have a carbazolyl group directly bonded to the naphthobenzofuran nucleus. Unlike the compound represented by Formula 1, which exhibits high electron mobility, the presence of the carbazolyl group raises the HOMO level, thereby resulting in an energy level unsuitable for the light-emitting layer region.

Furthermore, as can be seen from Table 7, the simultaneous inclusion of the heterocyclic compounds of Formula 1 and Formula 3 in the organic layer of an organic light-emitting device may improve the operating voltage, efficiency and Lifespan. These results suggest that the simultaneous inclusion of both compounds may lead to an exciplex phenomenon occurring between the two compounds. The exciplex phenomenon refers to the emission of energy corresponding to the HOMO level of the donor (p-Host) and the LUMO level of the acceptor (n-Host) through electron exchange between molecules having strong donor characteristics and molecules having strong acceptor characteristics. When a donor (p-Host) having excellent hole-transport capability and an acceptor (n-Host) having excellent electron-transport capability are used as host materials for the light-emitting layer, holes are injected into the p-Host and electrons are injected into the n-Host, thereby reducing the operating voltage and consequently improving the device lifetime. When the exciplex phenomenon occurs between the two molecules, reverse intersystem crossing (RISC) may take place, thereby increasing the internal quantum efficiency up to 100%.

### Manufacture of Organic Light-Emitting Device 2

A glass substrate coated with an indium tin oxide (ITO) film having a thickness of 1,500 Å was ultrasonically washed in distilled water. After completion of distilled water washing the substrate was ultrasonically washed with solvents such as acetone, methanol, and isopropyl alcohol. After drying, the substrate was subjected to ultraviolet ozone (UVO) treatment for 5 minutes in an ultraviolet (UV) cleaner. The substrate was then transferred to a plasma cleaner (PC) and subjected to plasma treatment under vacuum to adjust the ITO work function and remove any residual film. The substrate was then transferred to a thermal evaporation device for organic vapor deposition. A common layer of a hole injection layer, 2-TNATA (4,4′, 4″-tris[2-naphthyl(phenyl)amino]triphenylamine), and a hole transport layer (NPB) (N,N′-Di(1-naphthyl)-N,N′-diphenyl-(1,1′-biphenyl)-4,4′-diamine), were formed on the ITO transparent electrode (anode).

A light-emitting layer was thermally vacuum-deposited on the hole transport layer as follows. The light-emitting layer was deposited using the compounds shown in Table 8 as a red host, and 2 wt % of a red phosphorescent dopant, (piq)₂Ir(acac), was doped into the host to form a layer having a thickness of 400 Å.

Subsequently, 120 Å of Alq3 was deposited as an electron transport layer, 120 Å of Bphen was deposited as a charge generation layer, and 100 Å of MoO3 was deposited as a charge generation layer. A hole transport layer NPB (N,N′-Di(1-naphthyl)-N,N′-diphenyl-(1,1′-biphenyl)-4,4′-diamine) was formed.

The light-emitting layer was thermally vacuum-deposited on the hole transport layer as follows. The light-emitting layer was deposited using the compounds shown in Table 8 as a red host, and the host was doped with 2% red phosphorescent dopant (piq)2(Ir)(acac) to a 400 Å thickness.

Subsequently, Alq3 was deposited on the light-emitting layer in a thickness of 300 Å as an electron transport layer. Finally, lithium fluoride (LiF) was deposited on the electron transport layer in a thickness of 20 Å to form an electron injection layer. Then, an aluminum (Al) cathode was deposited on the electron injection layer in a thickness of 1,200 Å to form a cathode, thereby manufacturing an organic electroluminescent device. Meanwhile, all organic compounds required for the manufacture of the light-emitting device were purified by vacuum sublimation at 10-8 to 10-6 torr for each material and used in the manufacture of the light-emitting device.

The electroluminescence (EL) characteristics of the organic electroluminescent devices manufactured as described above were measured using the M7000 system from MacScience. Based on the measurement results, the lifespan (T95) was measured using a lifespan measurement system (M6000) manufactured by MacScience at a reference luminance of 6,000 cd/m2. The characteristics of the organic electroluminescent devices of the present invention are as shown in Table 8 below.

TABLE 8

| | | | | Operating | | |
|---|---|---|---|---|---|---|
| | First | Second | Ratio | voltage | Efficiency | Lifetime |
| | host | host | (N:P) | (V) | (cd/A) | (T95) |
| Comparative | A1 | A | 1:1 | 7.52 | 39.7 | 73 |
| Example 30 | | | | | | |
| Example 82 | | 21 | 1:1 | 6.04 | 127.3 | 185 |
| Comparative | A3 | B | 1:1 | 7.60 | 38.5 | 64 |
| Example 31 | | | | | | |
| Example 83 | | 177 | 1:1 | 6.28 | 88.2 | 148 |
| Comparative | A11 | C | 1:1 | 7.62 | 37.4 | 60 |
| Example 32 | | | | | | |
| Example 84 | | 137 | 1:1 | 6.34 | 85.3 | 152 |
| Comparative | A16 | D | 1:1 | 7.56 | 34.8 | 47 |
| Example 33 | | | | | | |
| Example 85 | | 50 | 1:1 | 6.22 | 110.0 | 143 |
| Comparative | A21 | E | 1:1 | 7.84 | 29.1 | 34 |
| Example 34 | | | | | | |
| Comparative | | K | 1:1 | 7.82 | 31.7 | 39 |
| Example 35 | | | | | | |
| Example 86 | | 377 | 1:1 | 6.44 | 97.2 | 148 |
| Comparative | A29 | F | 1:1 | 7.94 | 23.9 | 38 |
| Example 36 | | | | | | |
| Example 87 | | 371 | 1:1 | 6.26 | 82.4 | 137 |
| Comparative | | G | 1:1 | 7.90 | 25.4 | 43 |
| Example 37 | | | | | | |
| Example 88 | | 21 | 1:1 | 6.04 | 126.7 | 183 |
| Comparative | A69 | H | 1:1 | 7.74 | 27.2 | 47 |
| Example 38 | | | | | | |
| Example 89 | | 304 | 1:1 | 6.24 | 105.9 | 138 |
| Comparative | | I | 1:1 | 7.78 | 26.5 | 51 |
| Example 39 | | | | | | |
| Example 90 | | 280 | 1:1 | 6.67 | 85.7 | 120 |
| Comparative | A66 | L | 1:1 | 7.64 | 35.1 | 30 |
| Example 40 | | | | | | |
| Example 91 | | 220 | 1:1 | 6.81 | 87.4 | 115 |
| Comparative | A74 | M | 1:1 | 7.88 | 21.9 | 34 |
| Example 41 | | | | | | |
| Example 92 | | 195 | 1:1 | 6.40 | 86.5 | 134 |
| Comparative | | N | 1:1 | 7.76 | 20.8 | 43 |
| Example 42 | | | | | | |
| Example 93 | | 387 | 1:1 | 6.36 | 100.5 | 129 |
| Comparative | A99 | O | 1:1 | 7.70 | 25.7 | 51 |
| Example 43 | | | | | | |
| Example 94 | | 44 | 1:1 | 6.16 | 116.2 | 175 |
| Example 95 | A41 | 7 | 1:1 | 6.06 | 124.0 | 175 |
| Example 96 | | 9 | 1:1 | 6.10 | 126.1 | 203 |
| Example 97 | | 20 | 1:1 | 6.14 | 126.5 | 194 |
| Example 98 | A81 | 27 | 1:1 | 6.00 | 130.2 | 221 |
| Example 99 | | 30 | 1:1 | 6.06 | 126.5 | 212 |
| Example 100 | | 35 | 1:1 | 6.10 | 117.0 | 161 |
| Example 101 | | 61 | 1:1 | 6.24 | 112.1 | 134 |
| Example 102 | | 77 | 1:1 | 6.28 | 124.0 | 180 |
| Example 103 | A1 | 81 | 1:1 | 6.04 | 128.6 | 180 |
| Example 104 | | 99 | 1:1 | 6.04 | 131.0 | 212 |
| Example 105 | | 127 | 1:1 | 6.18 | 127.3 | 194 |
| Example 106 | | 175 | 1:1 | 6.32 | 87.4 | 138 |
| Example 107 | | 205 | 1:1 | 6.85 | 85.7 | 111 |
| Example 108 | A11 | 230 | 1:1 | 6.71 | 84.9 | 111 |
| Example 109 | | 258 | 1:1 | 6.45 | 89.8 | 148 |
| Example 110 | | 294 | 1:1 | 6.79 | 95.2 | 138 |
| Example 111 | | 299 | 1:1 | 6.71 | 93.1 | 120 |
| Example 112 | | 301 | 1:1 | 6.00 | 122.0 | 161 |
| Example 113 | | 307 | 1:1 | 6.06 | 131.4 | 194 |
| Example 114 | A74 | 319 | 1:1 | 5.98 | 133.5 | 212 |
| Example 115 | | 331 | 1:1 | 6.00 | 131.9 | 208 |
| Example 116 | | 346 | 1:1 | 6.24 | 88.6 | 134 |
| Example 117 | | 390 | 1:1 | 6.16 | 92.7 | 247 |
| Example 118 | | 396 | 1:1 | 6.06 | 124.4 | 328 |

As shown in Table 8 above, when an organic light-emitting device was manufactured by stacking two light-emitting layers (a two-stack device) including both the compound represented by Formula 1 and the compound represented by Formula 3 of the present invention, the light-emitting layers were deposited twice, and the device exhibited increased efficiency compared to the single-stack device.

Similar to the single-stack organic light-emitting device 1, in the organic light-emitting device including two stacks, it was confirmed that by incorporating the compounds represented by Formulae 1 and 3 of the present invention into the organic layer of the device, the operating voltage could be significantly improved due to increased hole mobility. Furthermore, the efficiency was also improved due to reduced current leakage through electron blocking and enhanced electron confinement.

The light-emitting devices of the examples provided T95 lifespan characteristics of 111 or higher, particularly 150 or higher, an operating voltage of 6.85 V or lower, and an operating efficiency of 82.4 cd/A or higher. In contrast, the light-emitting devices of the comparative examples exhibited a T95 lifespan of 72 or less, an operating voltage of 7.52 V or higher, and an operating efficiency of 39.6 cd/A or less.

The contents described above are merely examples of applying the principles of the present disclosure, and other configurations may be further included without departing from the scope of the present disclosure.

What is claimed is:

1. A heterocyclic compound represented by Formula 1: 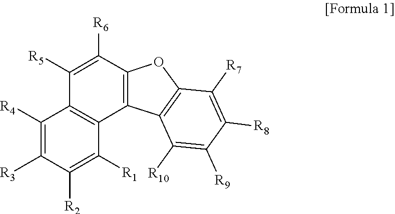
wherein one of R₁ to R₄ is an N-Het represented by Formula 2: 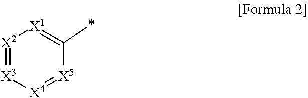
wherein X₁ to X₅ are each independently N or CR₁₁,
one of R₅ to R₁₀ is Ar₁,
Ar₁ is a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms,
R₁₁ is selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups are linked to form a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms,
provided that at least two of X₁ to X₅ are N,
the remainders of R₁ to R₄ that are not N-Het and the remainders of R₅ to R₁₀ that are not Ar₁ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups are linked to form a substituted or unsubstituted 5- to 9-membered saturated or unsaturated ring.
R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and
* represents a bonding site to an adjacent atom.

2. The heterocyclic compound according to claim 1, wherein the heterocyclic compound is represented by any one of Formulae 1-1 to 1-2: 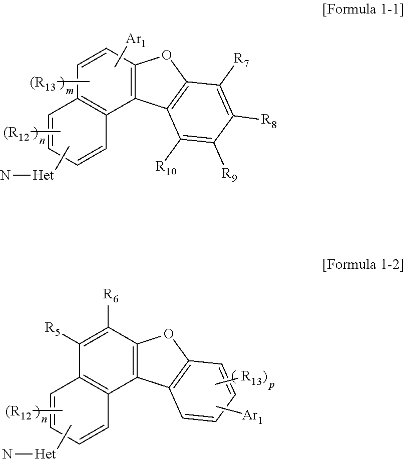
wherein, in Formulae 1-1 to 1-2, R₅ to R₁₀, N-Het, and Ar₁ have the same definitions as described in claims 1,
R₁₂ and R₁₃ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(—O)RR′, or adjacent groups are linked to form a substituted or unsubstituted 5- to 9-membered saturated or unsaturated ring,
R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms,
n and p are each independently 0 or an integer from 1 to 3,
m is 0 or 1,
when n is 2 or 3, a plurality of R₁₂ groups are the same or different, and
when p is 2 or 3, a plurality of R₁₃ groups are the same or different.

3. The heterocyclic compound according to claim 1, wherein the N-Het is represented by any one of Formulae 2-1 to 2-4 below: 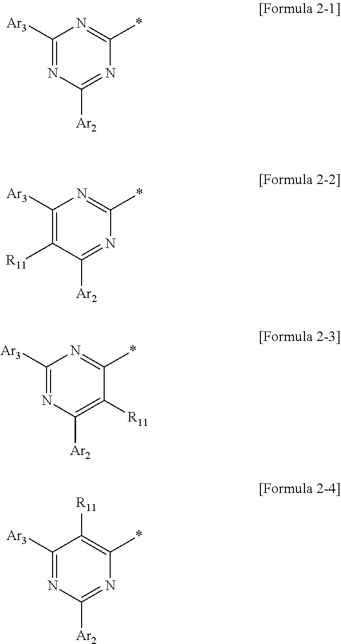
in Formulae 2-1 to 2-4, R₁₁ has the same definition as described in claim 1, and
Ar₂ or Ar₃ are each independently a substituted or unsubstituted aryl group having 6 to 60 carbon atoms, a substituted or unsubstituted heterocycloalkyl group having 2 to 60 carbon atoms; or a substituted or unsubstituted heteroaryl group having 2 to 60 carbon atoms.

4. The heterocyclic compound according to claim 1, wherein the N-Het is represented by any one of Formulae 2-5-1 to 2-5-5, Formulae 2-6-1 to 2-6-5, and Formula 2-7: 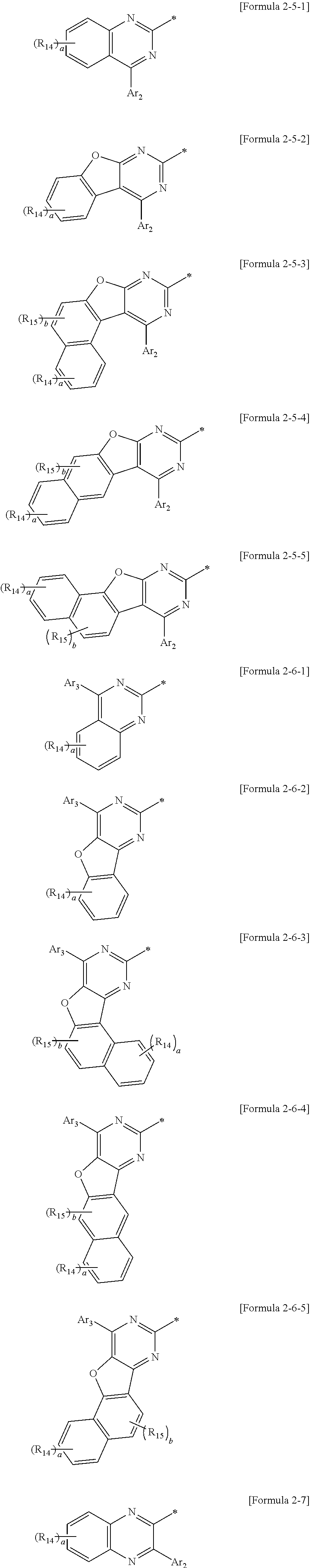
wherein, in Formula 2-5-1 to 2-5-4, Formula 2-6-1 to 2-6-5 and Formula 2-7, Ar₂ or Ar₃ is each independently a substituted or unsubstituted aryl group having 6 to 60 carbon atoms; a substituted or unsubstituted heterocycloalkyl group having 2 to 60 carbon atoms; or a substituted or unsubstituted heteroaryl group having 2 to 60 carbon atoms,
R₁₄ and R₁₅ are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(—O)RR′, or adjacent groups are linked to form a substituted or unsubstituted 5- to 9-membered saturated or unsaturated ring,
R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and
a is O or an integer from 1 to 4,
b is 0, 1, or 2,
when a is an integer from 2 to 4, a plurality of R₁₄ groups are the same or different, and
when b is 2, a plurality of R₁₅ groups are the same or different.

5. The heterocyclic compound according to claim 1, wherein Ar₁ is a substituted or unsubstituted aryl group having 6 to 40 carbon atoms; a substituted or unsubstituted heterocycloalkyl group having 2 to 40 carbon atoms; or a substituted or unsubstituted heteroaryl group having 2 to 40 carbon atoms.

6. The heterocyclic compound according to claim 1, wherein R₁₁ is each independently hydrogen, deuterium, a substituted or unsubstituted aryl group having 6 to 40 carbon atoms, a substituted or unsubstituted heterocycloalkyl group having 2 to 40 carbon atoms, or a substituted or unsubstituted heteroaryl group having 2 to 40 carbon atoms.

7. The heterocyclic compound according to claim 1, wherein the deuterium content of the heterocyclic compound is 0%, or greater than 0% and not more than 100%.

8. The heterocyclic compound according to claim 1, wherein the heterocyclic compound is represented by one of formulae below:

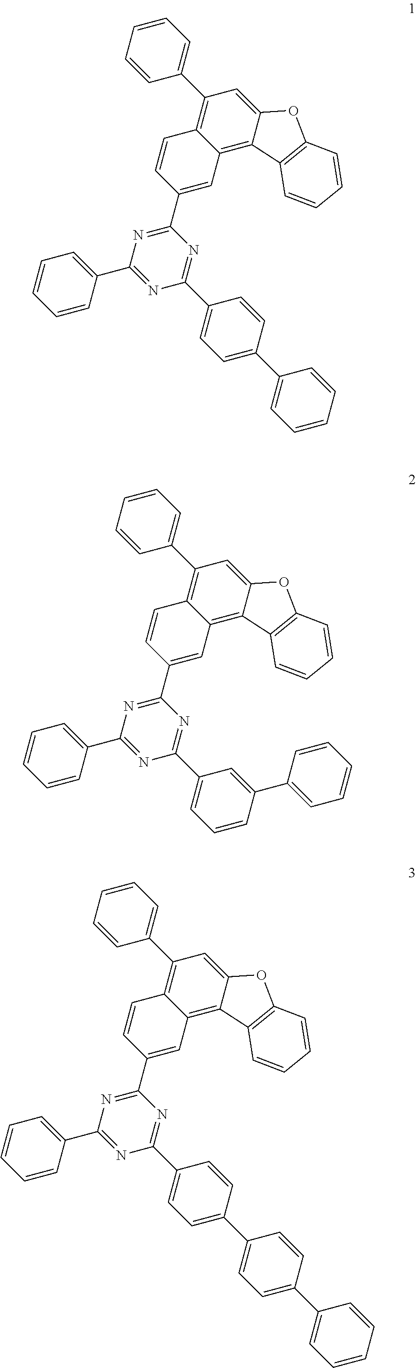 [SMILES: c1ccc(-c2ccc(-c3nc(-c4ccccc4)nc(-c4ccc5c(-c6ccccc6)cc6oc7ccccc7c6c5c4)n3)cc2)cc1 ; c1ccc(-c2cccc(-c3nc(-c4ccccc4)nc(-c4ccc5c(-c6ccccc6)cc6oc7ccccc7c6c5c4)n3)c2)cc1 ; c1ccc(-c2ccc(-c3ccc(-c4nc(-c5ccccc5)nc(-c5ccc6c(-c7ccccc7)cc7oc8ccccc8c7c6c5)n4)cc3)cc2)cc1]

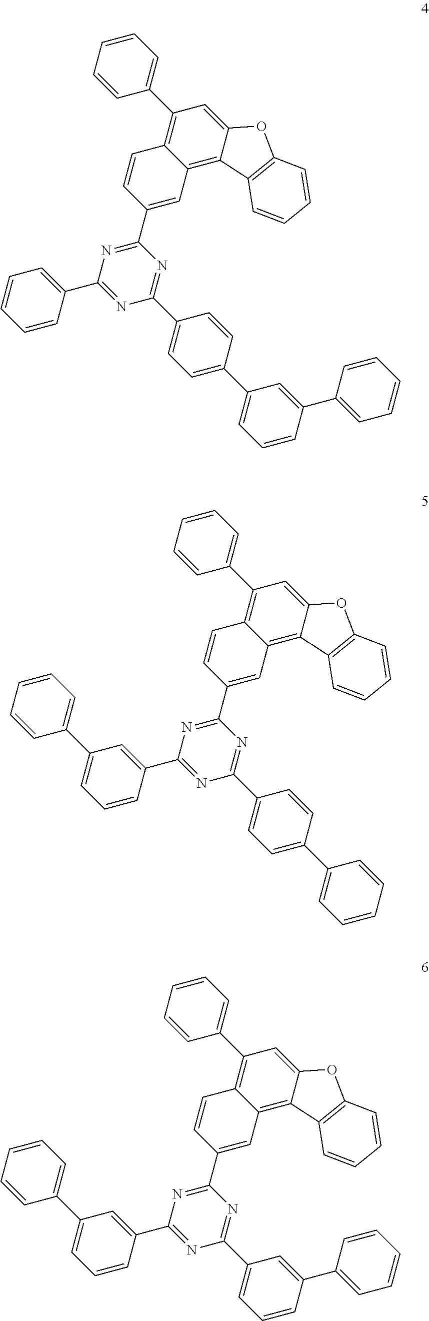 [SMILES: c1ccc(-c2cccc(-c3ccc(-c4nc(-c5ccccc5)nc(-c5ccc6c(-c7ccccc7)cc7oc8ccccc8c7c6c5)n4)cc3)c2)cc1 ; c1ccc(-c2ccc(-c3nc(-c4cccc(-c5ccccc5)c4)nc(-c4ccc5c(-c6ccccc6)cc6oc7ccccc7c6c5c4)n3)cc2)cc1 ; c1ccc(-c2cccc(-c3nc(-c4cccc(-c5ccccc5)c4)nc(-c4ccc5c(-c6ccccc6)cc6oc7ccccc7c6c5c4)n3)c2)cc1]

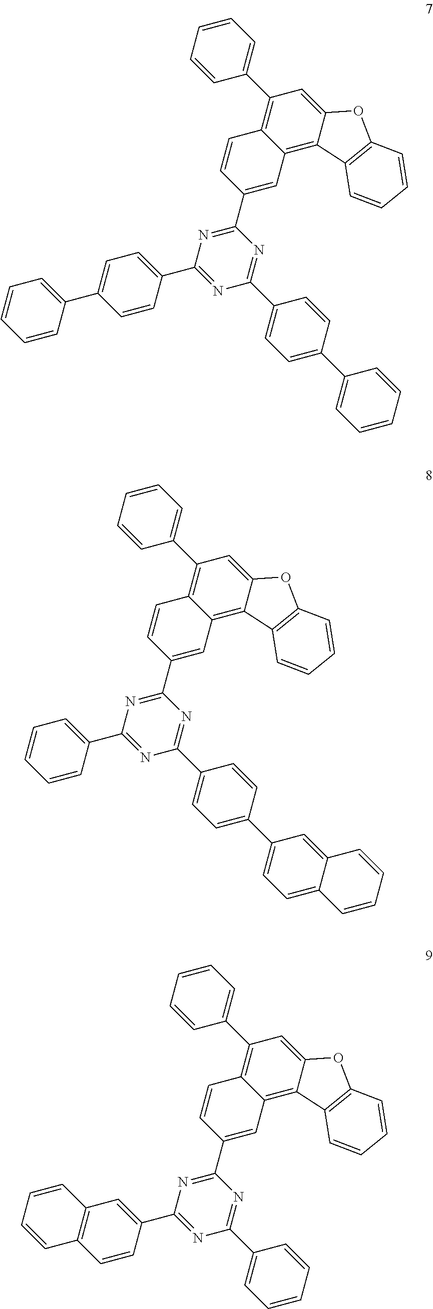 [SMILES: c1ccc(-c2ccc(-c3nc(-c4ccc(-c5ccccc5)cc4)nc(-c4ccc5c(-c6ccccc6)cc6oc7ccccc7c6c5c4)n3)cc2)cc1 ; c1ccc(-c2nc(-c3ccc(-c4ccc5ccccc5c4)cc3)nc(-c3ccc4c(-c5ccccc5)cc5oc6ccccc6c5c4c3)n2)cc1 ; c1ccc(-c2nc(-c3ccc4ccccc4c3)nc(-c3ccc4c(-c5ccccc5)cc5oc6ccccc6c5c4c3)n2)cc1]

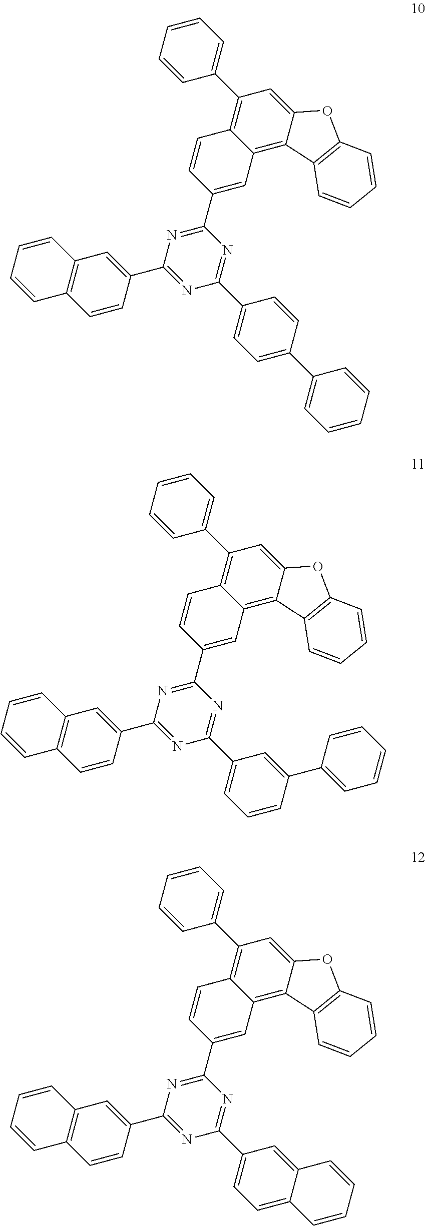 [SMILES: c1ccc(-c2ccc(-c3nc(-c4ccc5ccccc5c4)nc(-c4ccc5c(-c6ccccc6)cc6oc7ccccc7c6c5c4)n3)cc2)cc1 ; c1ccc(-c2cccc(-c3nc(-c4ccc5ccccc5c4)nc(-c4ccc5c(-c6ccccc6)cc6oc7ccccc7c6c5c4)n3)c2)cc1 ; c1ccc(-c2cc3oc4ccccc4c3c3cc(-c4nc(-c5ccc6ccccc6c5)nc(-c5ccc6ccccc6c5)n4)ccc23)cc1]

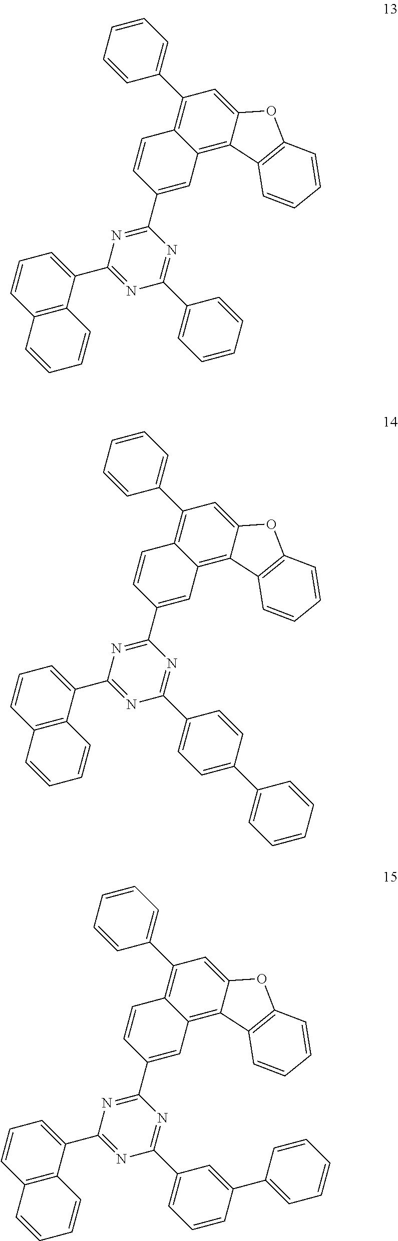 [SMILES: c1ccc(-c2nc(-c3ccc4c(-c5ccccc5)cc5oc6ccccc6c5c4c3)nc(-c3cccc4ccccc34)n2)cc1 ; c1ccc(-c2ccc(-c3nc(-c4ccc5c(-c6ccccc6)cc6oc7ccccc7c6c5c4)nc(-c4cccc5ccccc45)n3)cc2)cc1 ; c1ccc(-c2cccc(-c3nc(-c4ccc5c(-c6ccccc6)cc6oc7ccccc7c6c5c4)nc(-c4cccc5ccccc45)n3)c2)cc1]

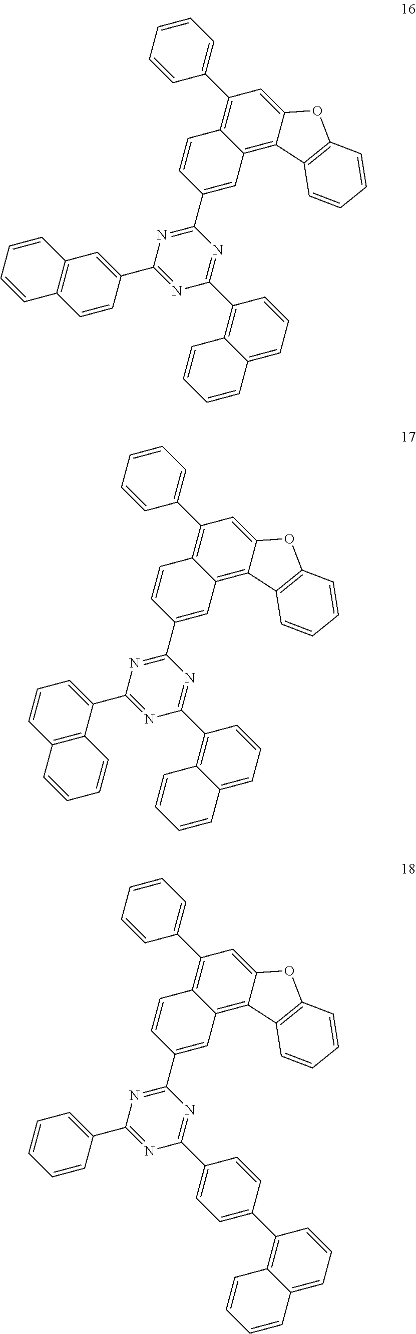 [SMILES: c1ccc(-c2cc3oc4ccccc4c3c3cc(-c4nc(-c5ccc6ccccc6c5)nc(-c5cccc6ccccc56)n4)ccc23)cc1 ; c1ccc(-c2cc3oc4ccccc4c3c3cc(-c4nc(-c5cccc6ccccc56)nc(-c5cccc6ccccc56)n4)ccc23)cc1 ; c1ccc(-c2nc(-c3ccc(-c4cccc5ccccc45)cc3)nc(-c3ccc4c(-c5ccccc5)cc5oc6ccccc6c5c4c3)n2)cc1]

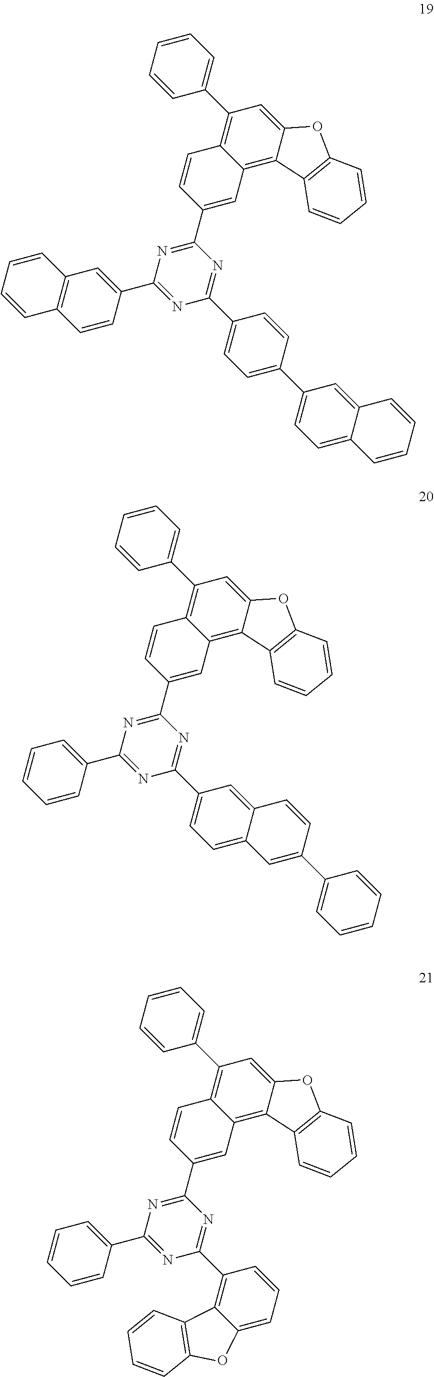 [SMILES: c1ccc(-c2cc3oc4ccccc4c3c3cc(-c4nc(-c5ccc(-c6ccc7ccccc7c6)cc5)nc(-c5ccc6ccccc6c5)n4)ccc23)cc1 ; c1ccc(-c2ccc3cc(-c4nc(-c5ccccc5)nc(-c5ccc6c(-c7ccccc7)cc7oc8ccccc8c7c6c5)n4)ccc3c2)cc1 ; c1ccc(-c2nc(-c3ccc4c(-c5ccccc5)cc5oc6ccccc6c5c4c3)nc(-c3cccc4oc5ccccc5c34)n2)cc1]

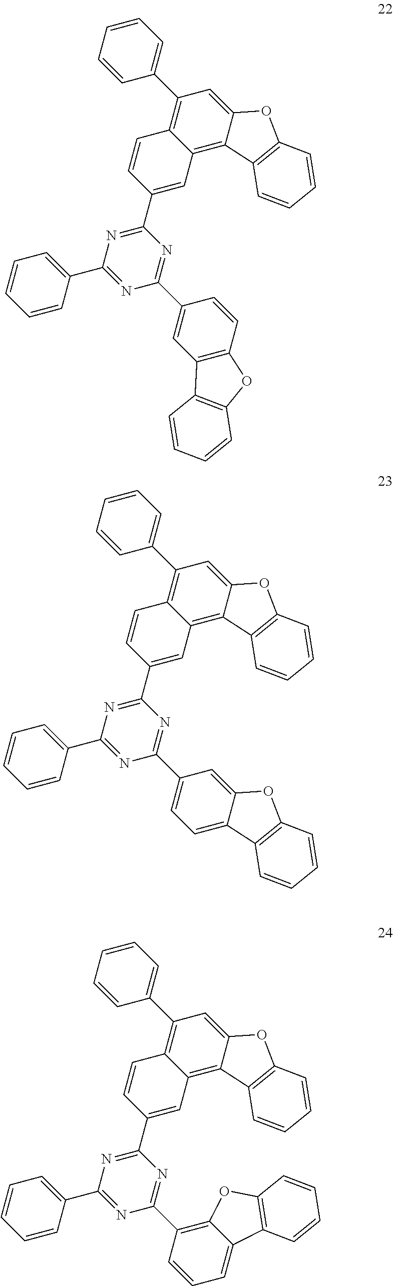 [SMILES: c1ccc(-c2nc(-c3ccc4oc5ccccc5c4c3)nc(-c3ccc4c(-c5ccccc5)cc5oc6ccccc6c5c4c3)n2)cc1 ; c1ccc(-c2nc(-c3ccc4c(c3)oc3ccccc34)nc(-c3ccc4c(-c5ccccc5)cc5oc6ccccc6c5c4c3)n2)cc1 ; c1ccc(-c2nc(-c3ccc4c(-c5ccccc5)cc5oc6ccccc6c5c4c3)nc(-c3cccc4c3oc3ccccc34)n2)cc1]

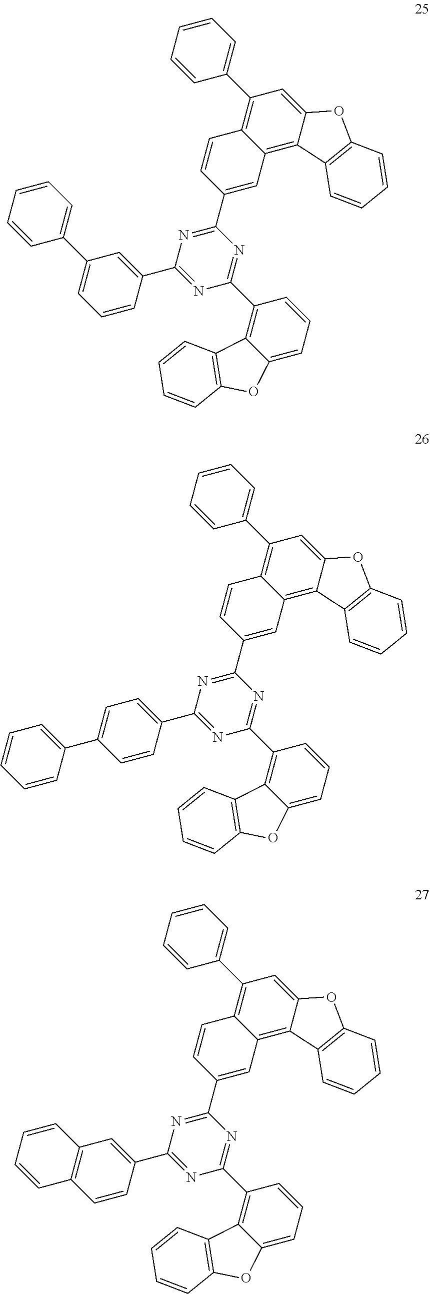 [SMILES: c1ccc(-c2cccc(-c3nc(-c4ccc5c(-c6ccccc6)cc6oc7ccccc7c6c5c4)nc(-c4cccc5oc6ccccc6c45)n3)c2)cc1 ; c1ccc(-c2ccc(-c3nc(-c4ccc5c(-c6ccccc6)cc6oc7ccccc7c6c5c4)nc(-c4cccc5oc6ccccc6c45)n3)cc2)cc1 ; c1ccc(-c2cc3oc4ccccc4c3c3cc(-c4nc(-c5ccc6ccccc6c5)nc(-c5cccc6oc7ccccc7c56)n4)ccc23)cc1]

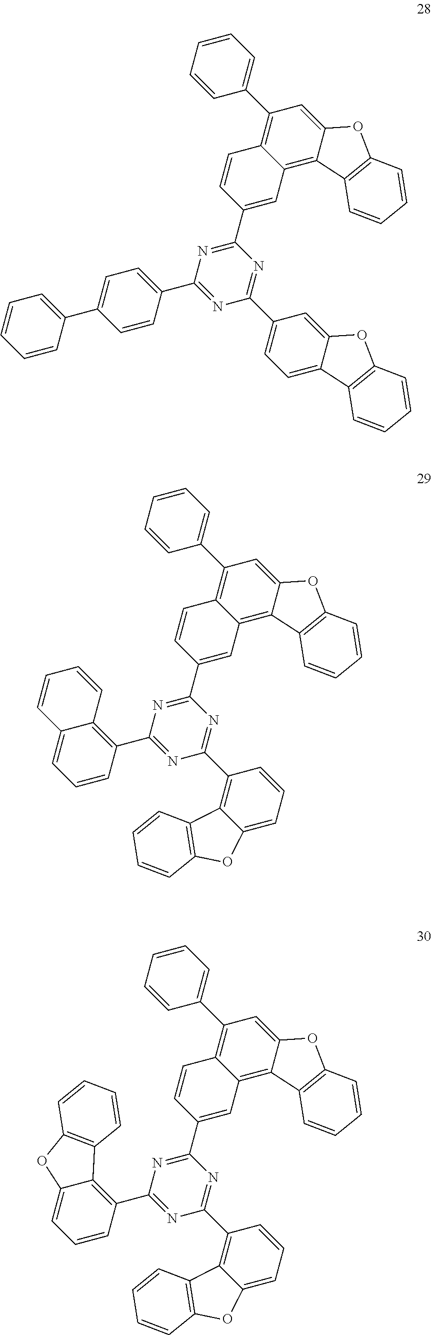 [SMILES: c1ccc(-c2ccc(-c3nc(-c4ccc5c(c4)oc4ccccc45)nc(-c4ccc5c(-c6ccccc6)cc6oc7ccccc7c6c5c4)n3)cc2)cc1 ; c1ccc(-c2cc3oc4ccccc4c3c3cc(-c4nc(-c5cccc6ccccc56)nc(-c5cccc6oc7ccccc7c56)n4)ccc23)cc1 ; c1ccc(-c2cc3oc4ccccc4c3c3cc(-c4nc(-c5cccc6oc7ccccc7c56)nc(-c5cccc6oc7ccccc7c56)n4)ccc23)cc1]

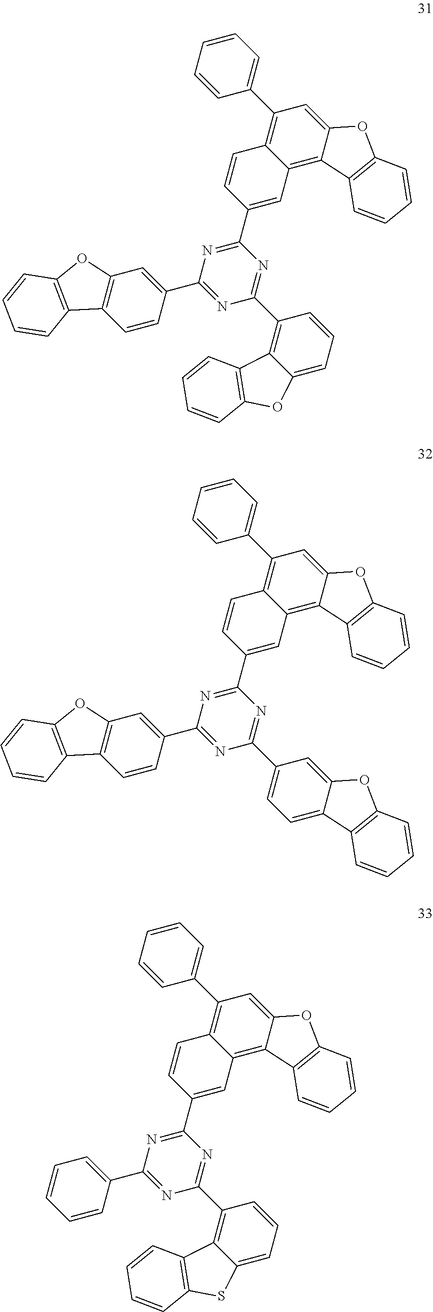 [SMILES: c1ccc(-c2cc3oc4ccccc4c3c3cc(-c4nc(-c5ccc6c(c5)oc5ccccc56)nc(-c5cccc6oc7ccccc7c56)n4)ccc23)cc1 ; c1ccc(-c2cc3oc4ccccc4c3c3cc(-c4nc(-c5ccc6c(c5)oc5ccccc56)nc(-c5ccc6c(c5)oc5ccccc56)n4)ccc23)cc1 ; c1ccc(-c2nc(-c3ccc4c(-c5ccccc5)cc5oc6ccccc6c5c4c3)nc(-c3cccc4sc5ccccc5c34)n2)cc1]

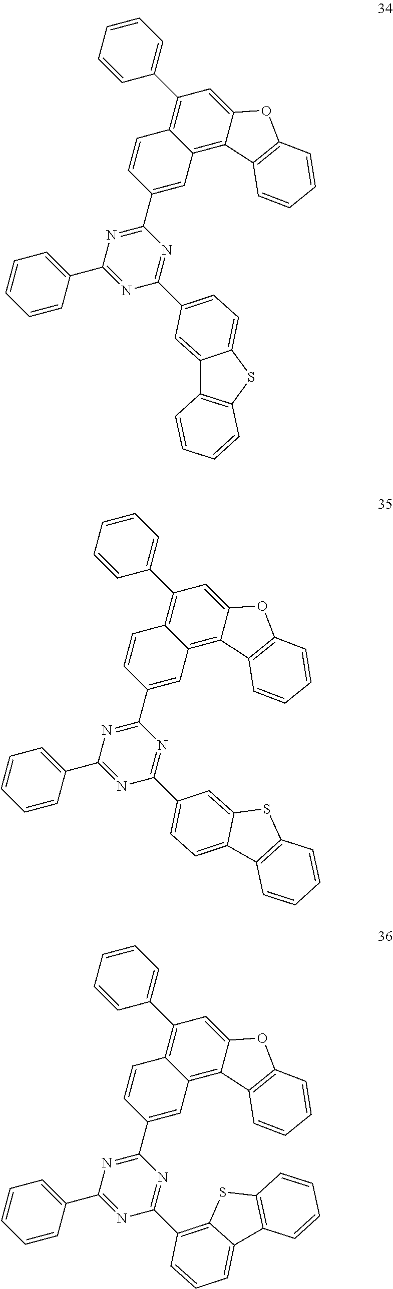 [SMILES: c1ccc(-c2nc(-c3ccc4sc5ccccc5c4c3)nc(-c3ccc4c(-c5ccccc5)cc5oc6ccccc6c5c4c3)n2)cc1 ; c1ccc(-c2nc(-c3ccc4c(c3)sc3ccccc34)nc(-c3ccc4c(-c5ccccc5)cc5oc6ccccc6c5c4c3)n2)cc1 ; c1ccc(-c2nc(-c3ccc4c(-c5ccccc5)cc5oc6ccccc6c5c4c3)nc(-c3cccc4c3sc3ccccc34)n2)cc1]

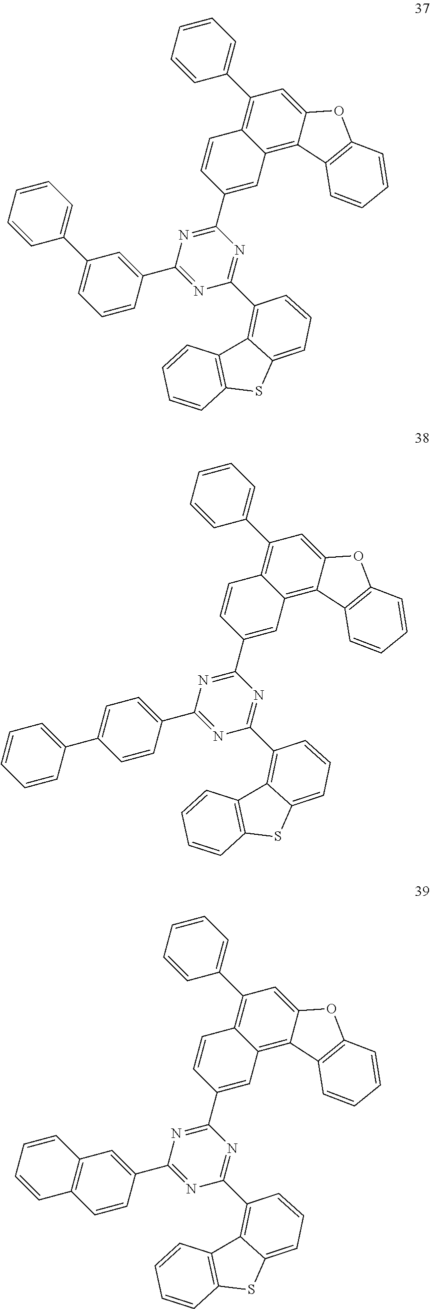 [SMILES: c1ccc(-c2cccc(-c3nc(-c4ccc5c(-c6ccccc6)cc6oc7ccccc7c6c5c4)nc(-c4cccc5sc6ccccc6c45)n3)c2)cc1 ; c1ccc(-c2ccc(-c3nc(-c4ccc5c(-c6ccccc6)cc6oc7ccccc7c6c5c4)nc(-c4cccc5sc6ccccc6c45)n3)cc2)cc1 ; c1ccc(-c2cc3oc4ccccc4c3c3cc(-c4nc(-c5ccc6ccccc6c5)nc(-c5cccc6sc7ccccc7c56)n4)ccc23)cc1]

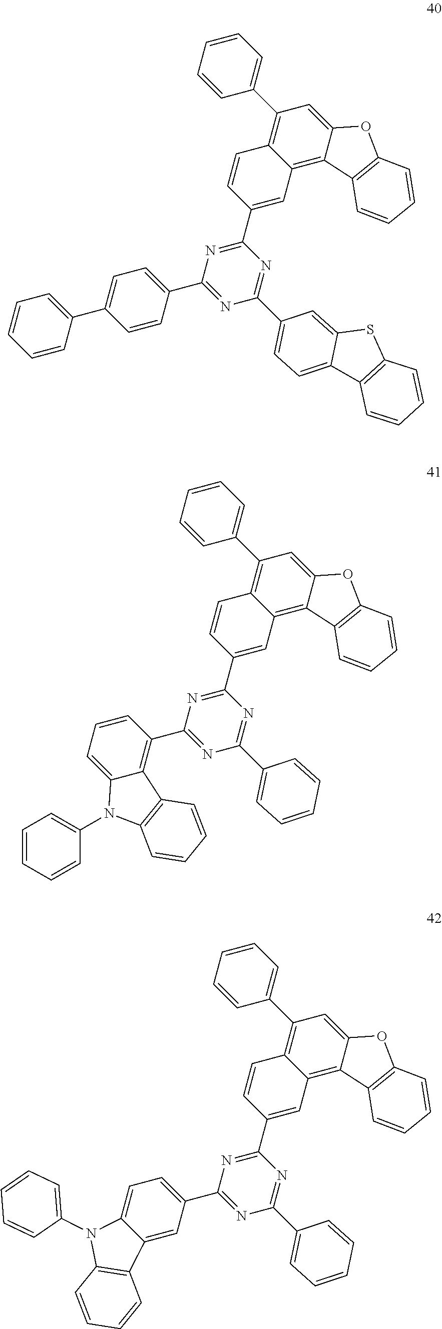 [SMILES: c1ccc(-c2ccc(-c3nc(-c4ccc5c(c4)sc4ccccc45)nc(-c4ccc5c(-c6ccccc6)cc6oc7ccccc7c6c5c4)n3)cc2)cc1 ; c1ccc(-c2nc(-c3ccc4c(-c5ccccc5)cc5oc6ccccc6c5c4c3)nc(-c3cccc4c3c3ccccc3n4-c3ccccc3)n2)cc1 ; c1ccc(-c2nc(-c3ccc4c(-c5ccccc5)cc5oc6ccccc6c5c4c3)nc(-c3ccc4c(c3)c3ccccc3n4-c3ccccc3)n2)cc1]

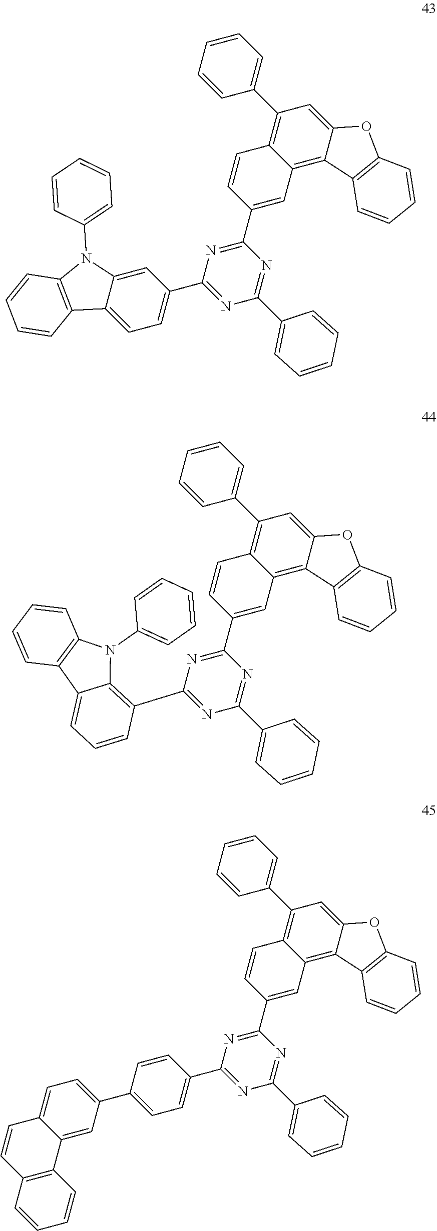 [SMILES: c1ccc(-c2nc(-c3ccc4c(-c5ccccc5)cc5oc6ccccc6c5c4c3)nc(-c3ccc4c5ccccc5n(-c5ccccc5)c4c3)n2)cc1 ; c1ccc(-c2nc(-c3ccc4c(-c5ccccc5)cc5oc6ccccc6c5c4c3)nc(-c3cccc4c5ccccc5n(-c5ccccc5)c34)n2)cc1 ; c1ccc(-c2nc(-c3ccc(-c4ccc5ccc6ccccc6c5c4)cc3)nc(-c3ccc4c(-c5ccccc5)cc5oc6ccccc6c5c4c3)n2)cc1]

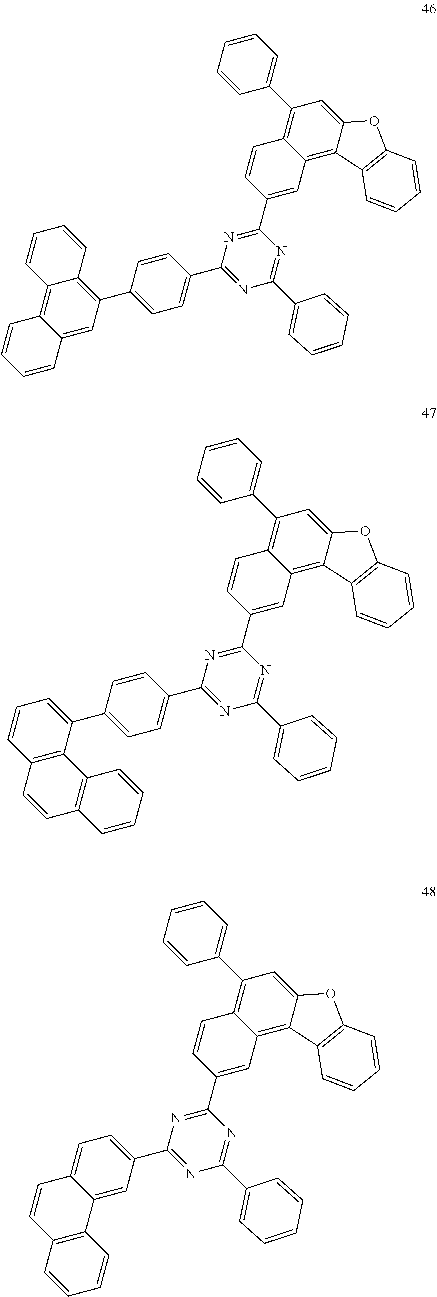 [SMILES: c1ccc(-c2nc(-c3ccc(-c4cc5ccccc5c5ccccc45)cc3)nc(-c3ccc4c(-c5ccccc5)cc5oc6ccccc6c5c4c3)n2)cc1 ; c1ccc(-c2nc(-c3ccc(-c4cccc5ccc6ccccc6c45)cc3)nc(-c3ccc4c(-c5ccccc5)cc5oc6ccccc6c5c4c3)n2)cc1 ; c1ccc(-c2nc(-c3ccc4ccc5ccccc5c4c3)nc(-c3ccc4c(-c5ccccc5)cc5oc6ccccc6c5c4c3)n2)cc1]

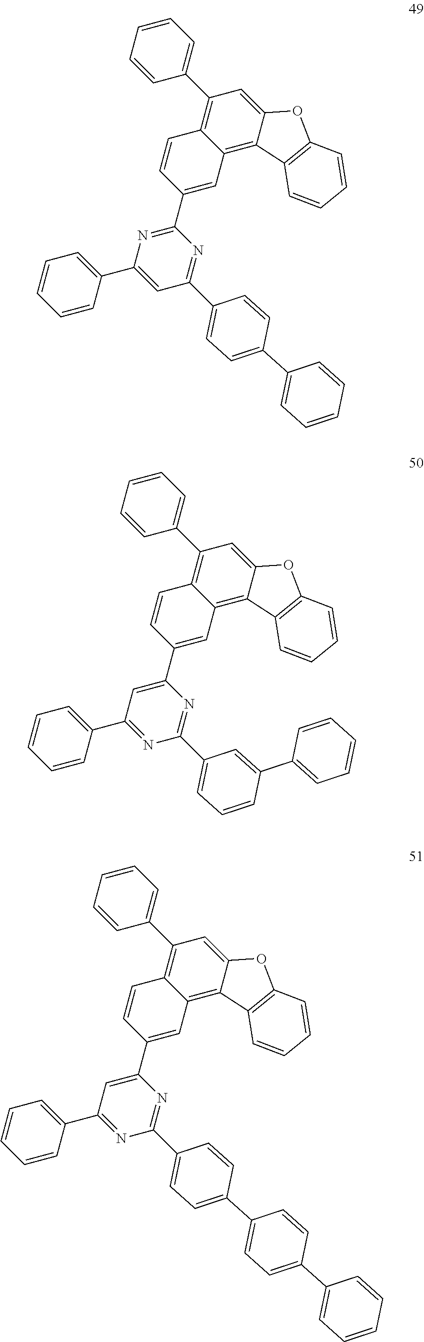 [SMILES: c1ccc(-c2ccc(-c3cc(-c4ccccc4)nc(-c4ccc5c(-c6ccccc6)cc6oc7ccccc7c6c5c4)n3)cc2)cc1 ; c1ccc(-c2cccc(-c3nc(-c4ccccc4)cc(-c4ccc5c(-c6ccccc6)cc6oc7ccccc7c6c5c4)n3)c2)cc1 ; c1ccc(-c2ccc(-c3ccc(-c4nc(-c5ccccc5)cc(-c5ccc6c(-c7ccccc7)cc7oc8ccccc8c7c6c5)n4)cc3)cc2)cc1]

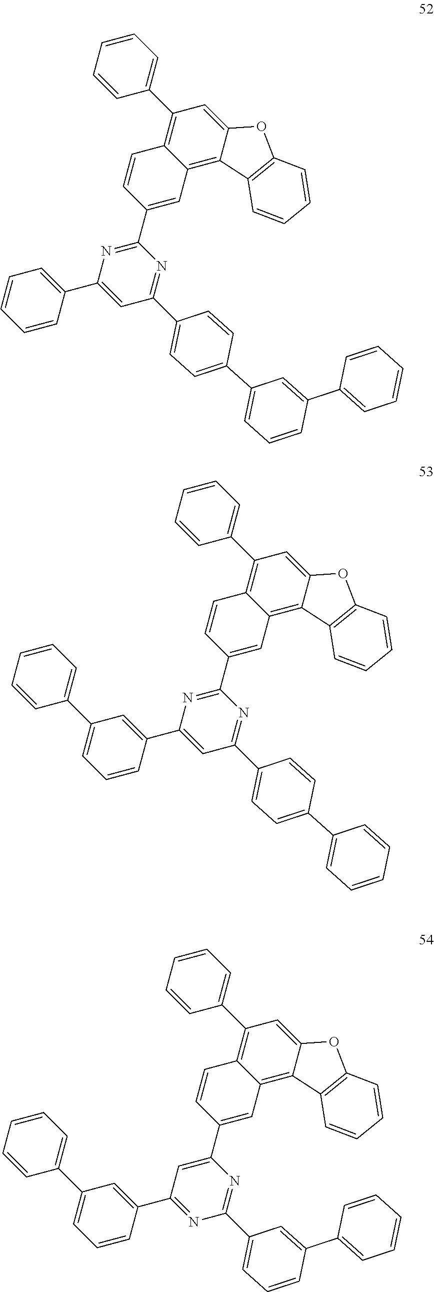 [SMILES: c1ccc(-c2cccc(-c3ccc(-c4cc(-c5ccccc5)nc(-c5ccc6c(-c7ccccc7)cc7oc8ccccc8c7c6c5)n4)cc3)c2)cc1 ; c1ccc(-c2ccc(-c3cc(-c4cccc(-c5ccccc5)c4)nc(-c4ccc5c(-c6ccccc6)cc6oc7ccccc7c6c5c4)n3)cc2)cc1 ; c1ccc(-c2cccc(-c3cc(-c4ccc5c(-c6ccccc6)cc6oc7ccccc7c6c5c4)nc(-c4cccc(-c5ccccc5)c4)n3)c2)cc1]

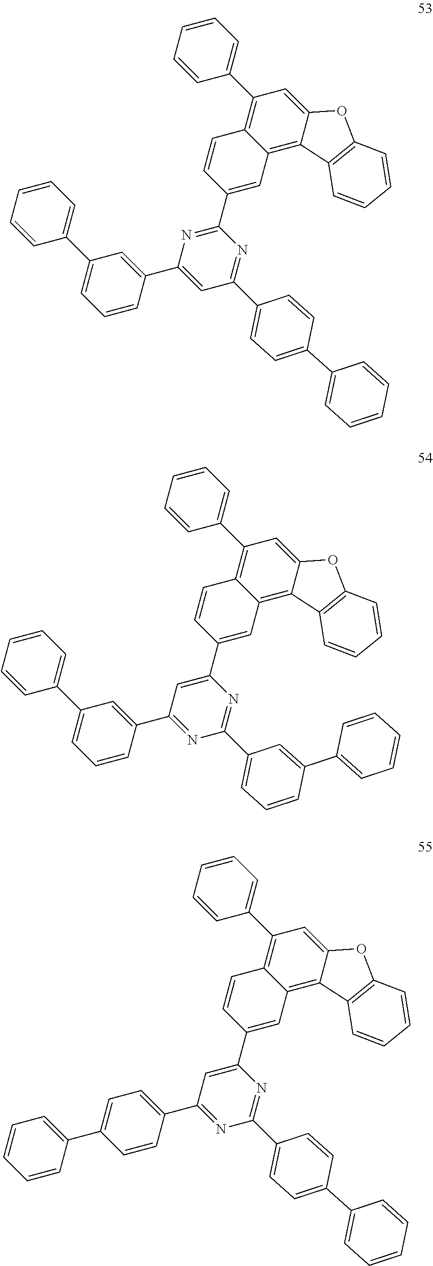 [SMILES: c1ccc(-c2ccc(-c3cc(-c4cccc(-c5ccccc5)c4)nc(-c4ccc5c(-c6ccccc6)cc6oc7ccccc7c6c5c4)n3)cc2)cc1 ; c1ccc(-c2cccc(-c3cc(-c4ccc5c(-c6ccccc6)cc6oc7ccccc7c6c5c4)nc(-c4cccc(-c5ccccc5)c4)n3)c2)cc1 ; c1ccc(-c2ccc(-c3cc(-c4ccc5c(-c6ccccc6)cc6oc7ccccc7c6c5c4)nc(-c4ccc(-c5ccccc5)cc4)n3)cc2)cc1]

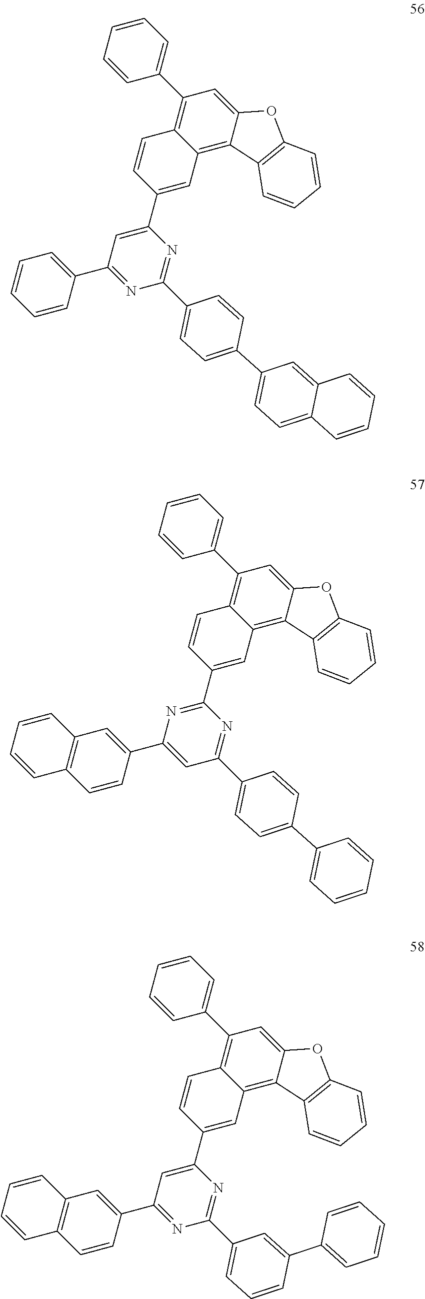 [SMILES: c1ccc(-c2cc(-c3ccc4c(-c5ccccc5)cc5oc6ccccc6c5c4c3)nc(-c3ccc(-c4ccc5ccccc5c4)cc3)n2)cc1 ; c1ccc(-c2ccc(-c3cc(-c4ccc5ccccc5c4)nc(-c4ccc5c(-c6ccccc6)cc6oc7ccccc7c6c5c4)n3)cc2)cc1 ; c1ccc(-c2cccc(-c3nc(-c4ccc5ccccc5c4)cc(-c4ccc5c(-c6ccccc6)cc6oc7ccccc7c6c5c4)n3)c2)cc1]

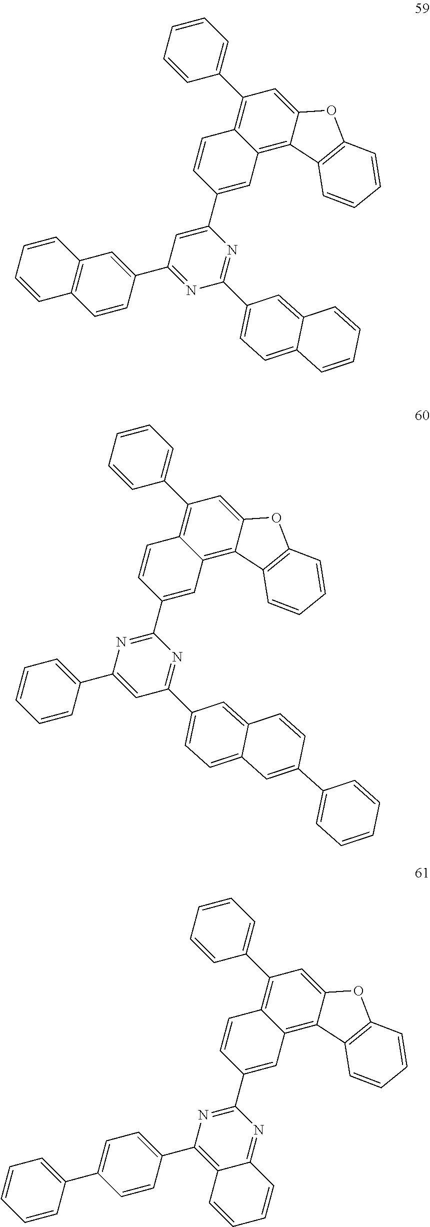 [SMILES: c1ccc(-c2cc3oc4ccccc4c3c3cc(-c4cc(-c5ccc6ccccc6c5)nc(-c5ccc6ccccc6c5)n4)ccc23)cc1 ; c1ccc(-c2ccc3cc(-c4cc(-c5ccccc5)nc(-c5ccc6c(-c7ccccc7)cc7oc8ccccc8c7c6c5)n4)ccc3c2)cc1 ; c1ccc(-c2ccc(-c3nc(-c4ccc5c(-c6ccccc6)cc6oc7ccccc7c6c5c4)nc4ccccc34)cc2)cc1]

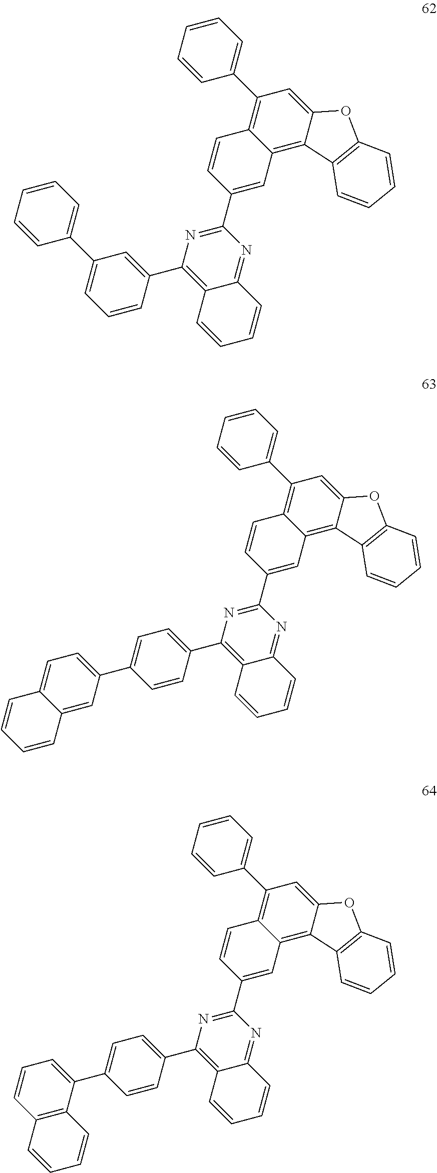 [SMILES: c1ccc(-c2cccc(-c3nc(-c4ccc5c(-c6ccccc6)cc6oc7ccccc7c6c5c4)nc4ccccc34)c2)cc1 ; c1ccc(-c2cc3oc4ccccc4c3c3cc(-c4nc(-c5ccc(-c6ccc7ccccc7c6)cc5)c5ccccc5n4)ccc23)cc1 ; c1ccc(-c2cc3oc4ccccc4c3c3cc(-c4nc(-c5ccc(-c6cccc7ccccc67)cc5)c5ccccc5n4)ccc23)cc1]

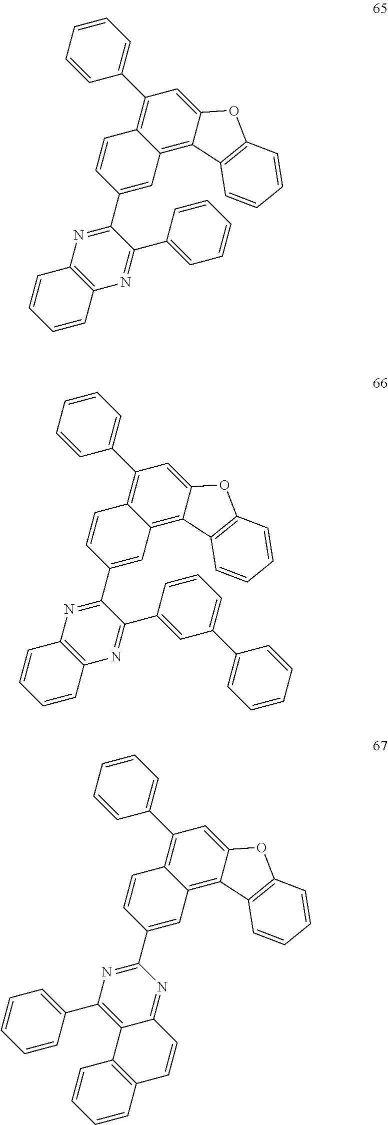 [SMILES: c1ccc(-c2nc3ccccc3nc2-c2ccc3c(-c4ccccc4)cc4oc5ccccc5c4c3c2)cc1 ; c1ccc(-c2cccc(-c3nc4ccccc4nc3-c3ccc4c(-c5ccccc5)cc5oc6ccccc6c5c4c3)c2)cc1 ; c1ccc(-c2cc3oc4ccccc4c3c3cc(-c4nc(-c5ccccc5)c5c(ccc6ccccc65)n4)ccc23)cc1]

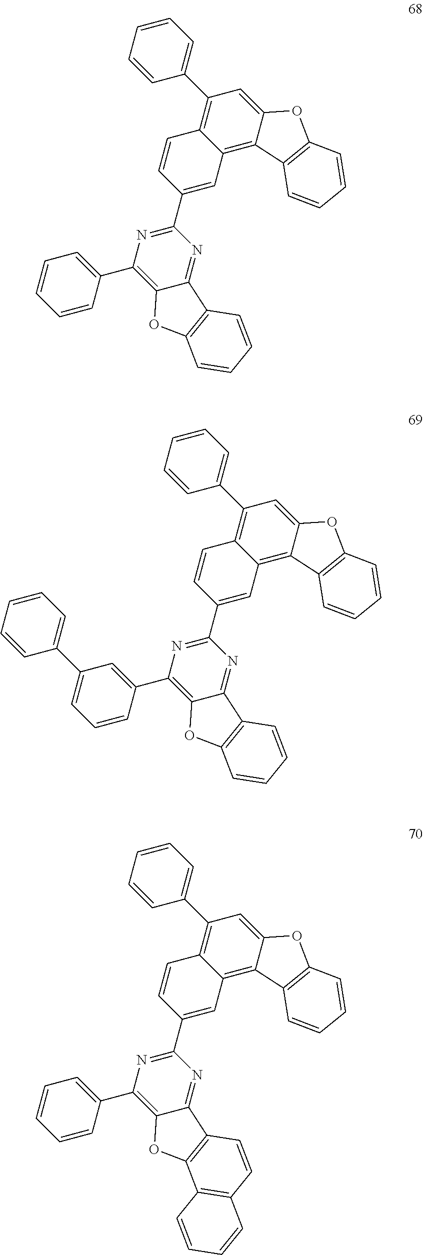 [SMILES: c1ccc(-c2cc3oc4ccccc4c3c3cc(-c4nc(-c5ccccc5)c5oc6ccccc6c5n4)ccc23)cc1 ; c1ccc(-c2cccc(-c3nc(-c4ccc5c(-c6ccccc6)cc6oc7ccccc7c6c5c4)nc4c3oc3ccccc34)c2)cc1 ; c1ccc(-c2cc3oc4ccccc4c3c3cc(-c4nc(-c5ccccc5)c5oc6c7ccccc7ccc6c5n4)ccc23)cc1]

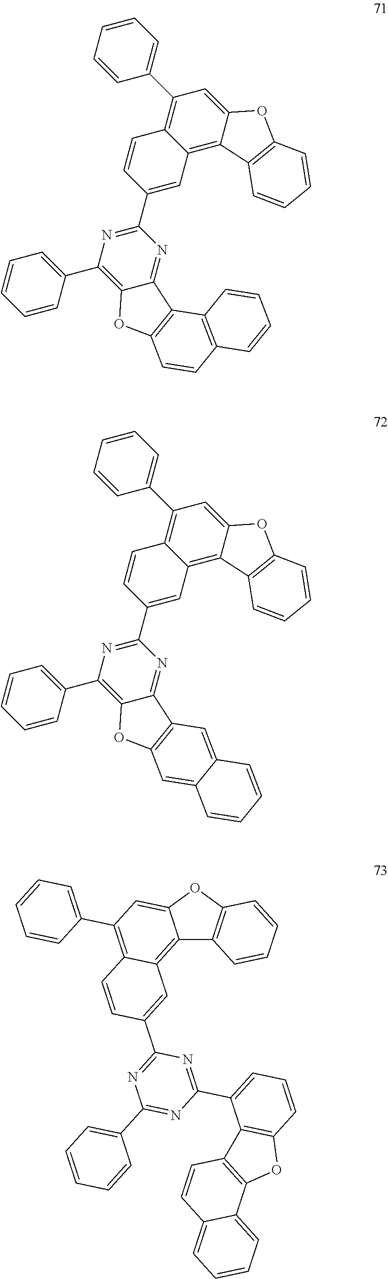 [SMILES: c1ccc(-c2cc3oc4ccccc4c3c3cc(-c4nc(-c5ccccc5)c5oc6ccc7ccccc7c6c5n4)ccc23)cc1 ; c1ccc(-c2cc3oc4ccccc4c3c3cc(-c4nc(-c5ccccc5)c5oc6cc7ccccc7cc6c5n4)ccc23)cc1 ; c1ccc(-c2nc(-c3ccc4c(-c5ccccc5)cc5oc6ccccc6c5c4c3)nc(-c3cccc4oc5c6ccccc6ccc5c34)n2)cc1]

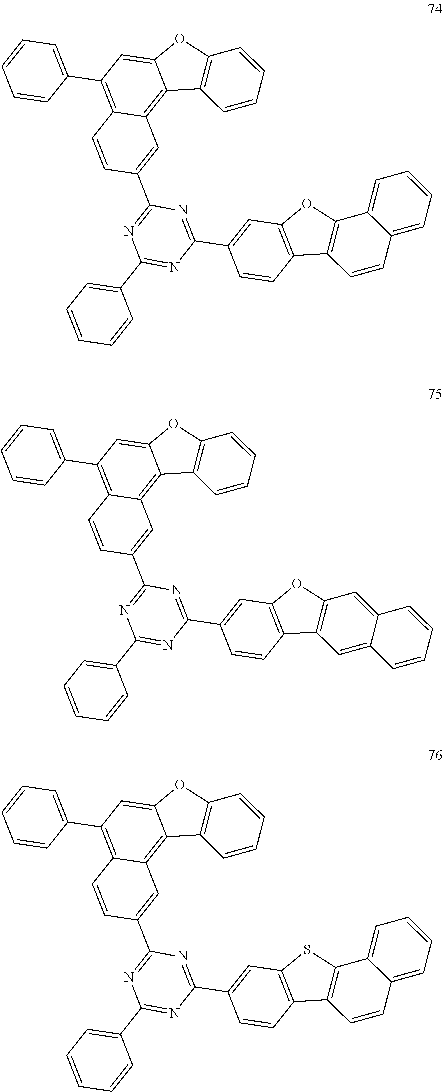 [SMILES: c1ccc(-c2nc(-c3ccc4c(c3)oc3c5ccccc5ccc43)nc(-c3ccc4c(-c5ccccc5)cc5oc6ccccc6c5c4c3)n2)cc1 ; c1ccc(-c2nc(-c3ccc4c(c3)oc3cc5ccccc5cc34)nc(-c3ccc4c(-c5ccccc5)cc5oc6ccccc6c5c4c3)n2)cc1 ; c1ccc(-c2nc(-c3ccc4c(c3)sc3c5ccccc5ccc43)nc(-c3ccc4c(-c5ccccc5)cc5oc6ccccc6c5c4c3)n2)cc1]

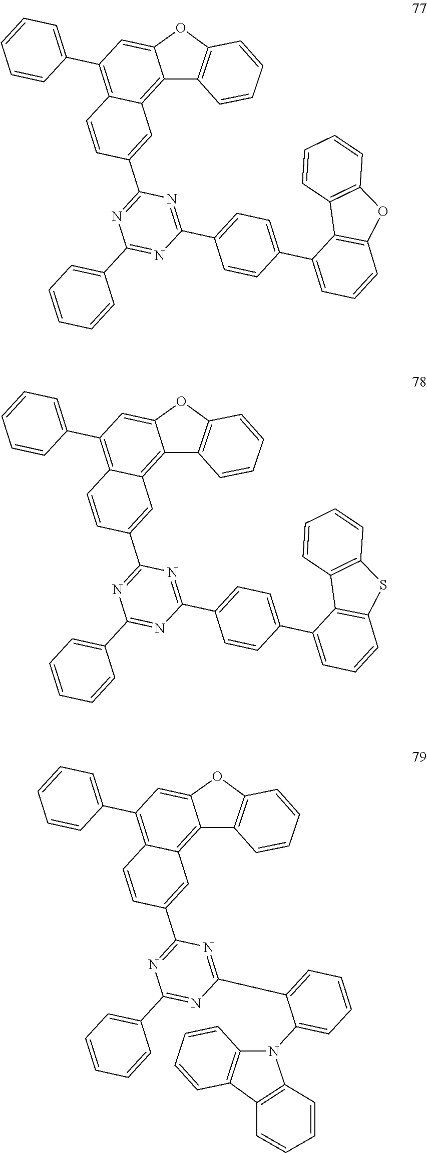 [SMILES: c1ccc(-c2nc(-c3ccc(-c4cccc5oc6ccccc6c45)cc3)nc(-c3ccc4c(-c5ccccc5)cc5oc6ccccc6c5c4c3)n2)cc1 ; c1ccc(-c2nc(-c3ccc(-c4cccc5sc6ccccc6c45)cc3)nc(-c3ccc4c(-c5ccccc5)cc5oc6ccccc6c5c4c3)n2)cc1 ; c1ccc(-c2nc(-c3ccc4c(-c5ccccc5)cc5oc6ccccc6c5c4c3)nc(-c3ccccc3-n3c4ccccc4c4ccccc43)n2)cc1]

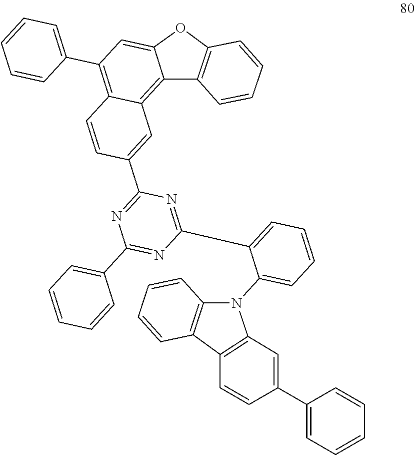 [SMILES: c1ccc(-c2ccc3c4ccccc4n(-c4ccccc4-c4nc(-c5ccccc5)nc(-c5ccc6c(-c7ccccc7)cc7oc8ccccc8c7c6c5)n4)c3c2)cc1]

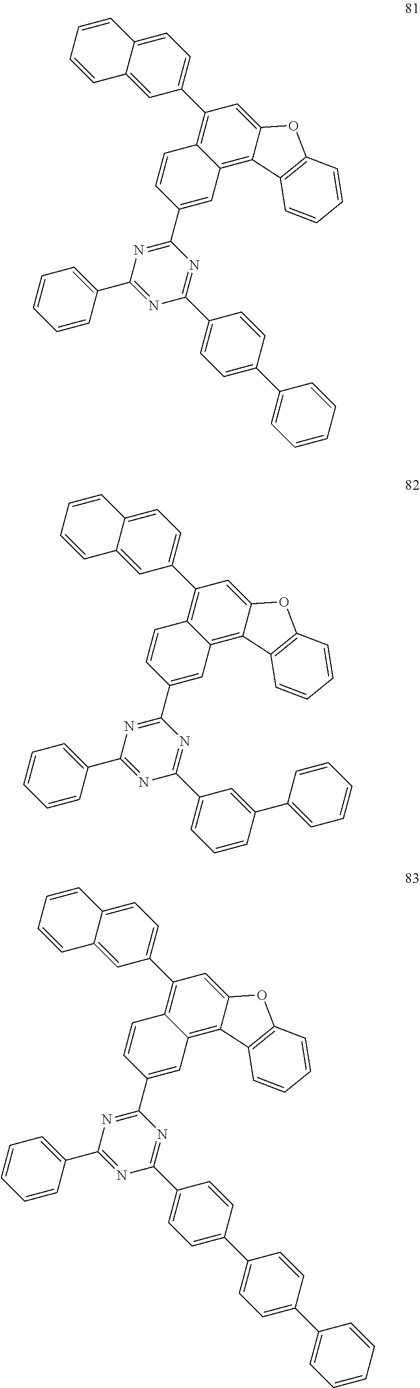 [SMILES: c1ccc(-c2ccc(-c3nc(-c4ccccc4)nc(-c4ccc5c(-c6ccc7ccccc7c6)cc6oc7ccccc7c6c5c4)n3)cc2)cc1 ; c1ccc(-c2cccc(-c3nc(-c4ccccc4)nc(-c4ccc5c(-c6ccc7ccccc7c6)cc6oc7ccccc7c6c5c4)n3)c2)cc1 ; c1ccc(-c2ccc(-c3ccc(-c4nc(-c5ccccc5)nc(-c5ccc6c(-c7ccc8ccccc8c7)cc7oc8ccccc8c7c6c5)n4)cc3)cc2)cc1]

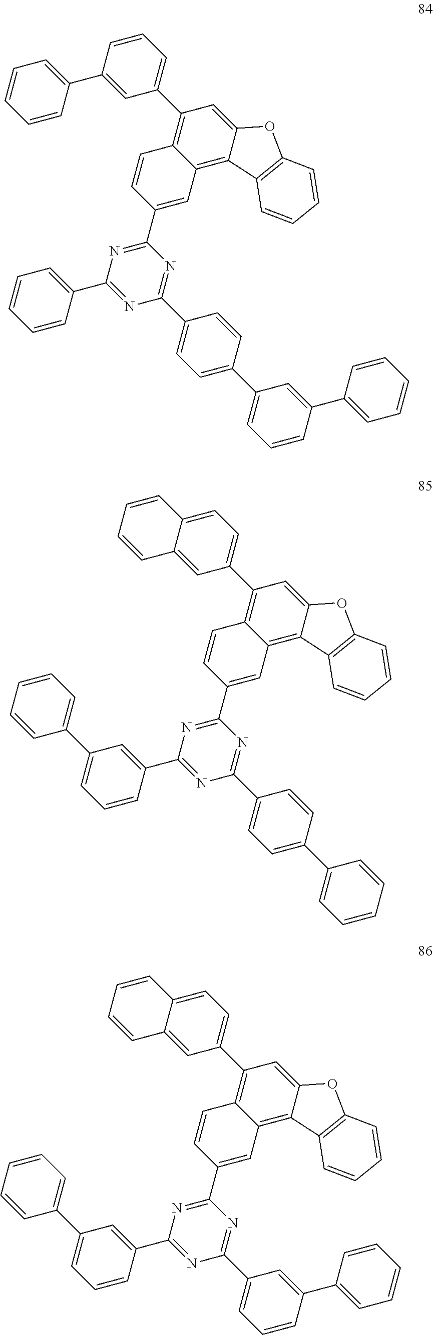 [SMILES: c1ccc(-c2cccc(-c3ccc(-c4nc(-c5ccccc5)nc(-c5ccc6c(-c7cccc(-c8ccccc8)c7)cc7oc8ccccc8c7c6c5)n4)cc3)c2)cc1 ; c1ccc(-c2ccc(-c3nc(-c4cccc(-c5ccccc5)c4)nc(-c4ccc5c(-c6ccc7ccccc7c6)cc6oc7ccccc7c6c5c4)n3)cc2)cc1 ; c1ccc(-c2cccc(-c3nc(-c4cccc(-c5ccccc5)c4)nc(-c4ccc5c(-c6ccc7ccccc7c6)cc6oc7ccccc7c6c5c4)n3)c2)cc1]

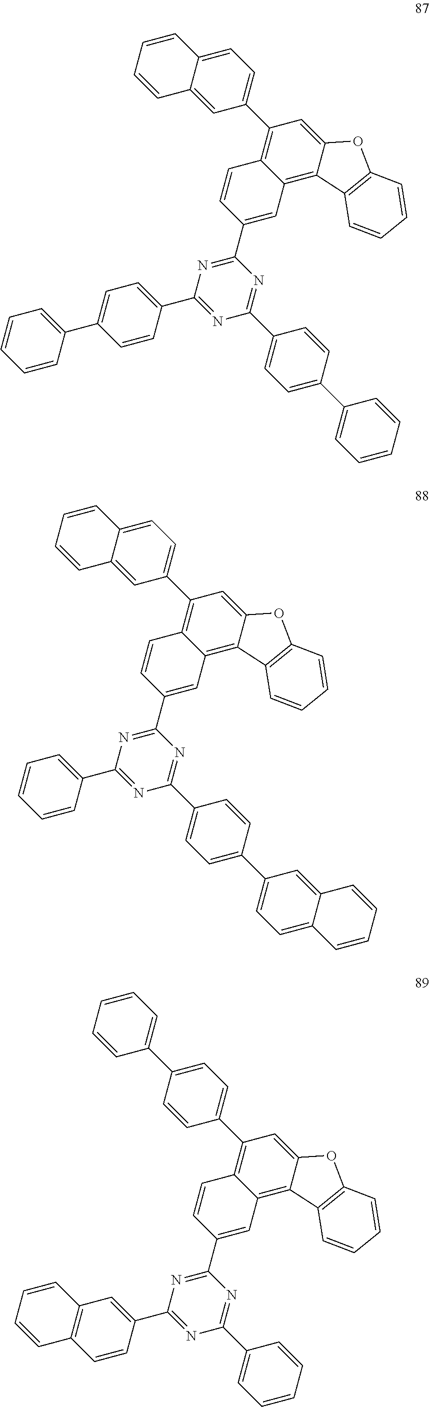 [SMILES: c1ccc(-c2ccc(-c3nc(-c4ccc(-c5ccccc5)cc4)nc(-c4ccc5c(-c6ccc7ccccc7c6)cc6oc7ccccc7c6c5c4)n3)cc2)cc1 ; c1ccc(-c2nc(-c3ccc(-c4ccc5ccccc5c4)cc3)nc(-c3ccc4c(-c5ccc6ccccc6c5)cc5oc6ccccc6c5c4c3)n2)cc1 ; c1ccc(-c2ccc(-c3cc4oc5ccccc5c4c4cc(-c5nc(-c6ccccc6)nc(-c6ccc7ccccc7c6)n5)ccc34)cc2)cc1]

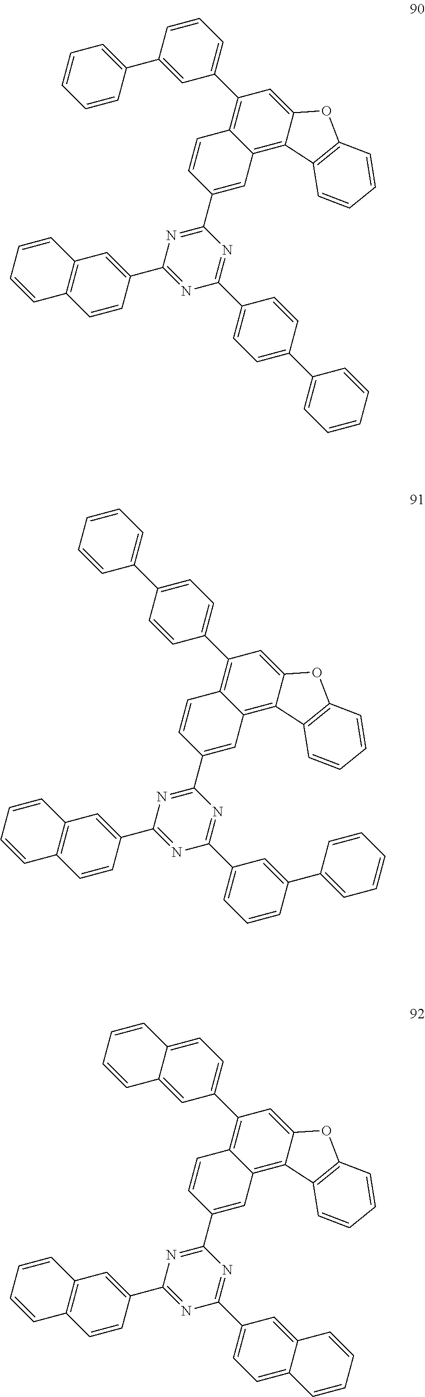 [SMILES: c1ccc(-c2ccc(-c3nc(-c4ccc5ccccc5c4)nc(-c4ccc5c(-c6cccc(-c7ccccc7)c6)cc6oc7ccccc7c6c5c4)n3)cc2)cc1 ; c1ccc(-c2ccc(-c3cc4oc5ccccc5c4c4cc(-c5nc(-c6cccc(-c7ccccc7)c6)nc(-c6ccc7ccccc7c6)n5)ccc34)cc2)cc1 ; c1ccc2cc(-c3nc(-c4ccc5ccccc5c4)nc(-c4ccc5c(-c6ccc7ccccc7c6)cc6oc7ccccc7c6c5c4)n3)ccc2c1]

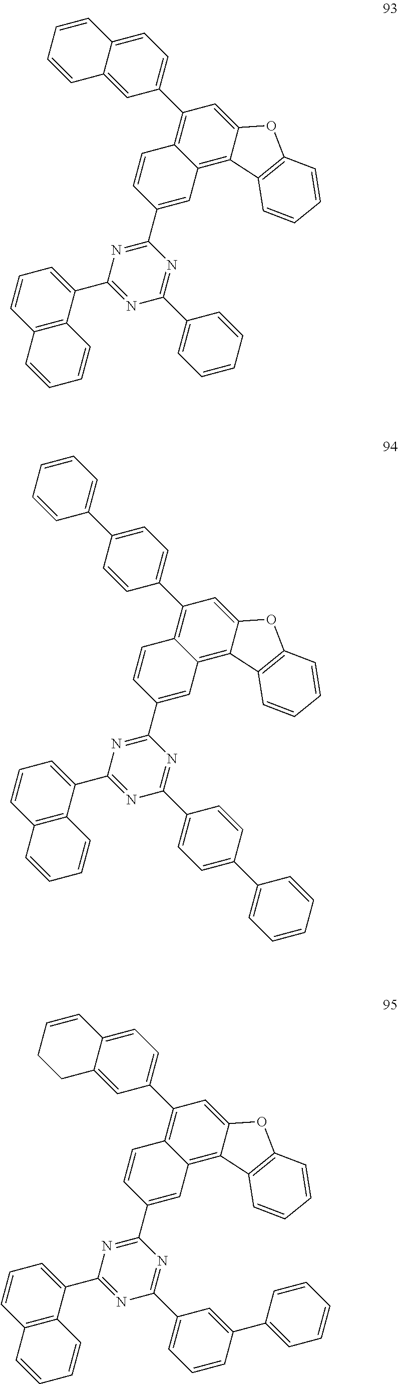 [SMILES: c1ccc(-c2nc(-c3ccc4c(-c5ccc6ccccc6c5)cc5oc6ccccc6c5c4c3)nc(-c3cccc4ccccc34)n2)cc1 ; c1ccc(-c2ccc(-c3nc(-c4ccc5c(-c6ccc(-c7ccccc7)cc6)cc6oc7ccccc7c6c5c4)nc(-c4cccc5ccccc45)n3)cc2)cc1 ; C1=Cc2ccc(-c3cc4oc5ccccc5c4c4cc(-c5nc(-c6cccc(-c7ccccc7)c6)nc(-c6cccc7ccccc67)n5)ccc34)cc2CC1]

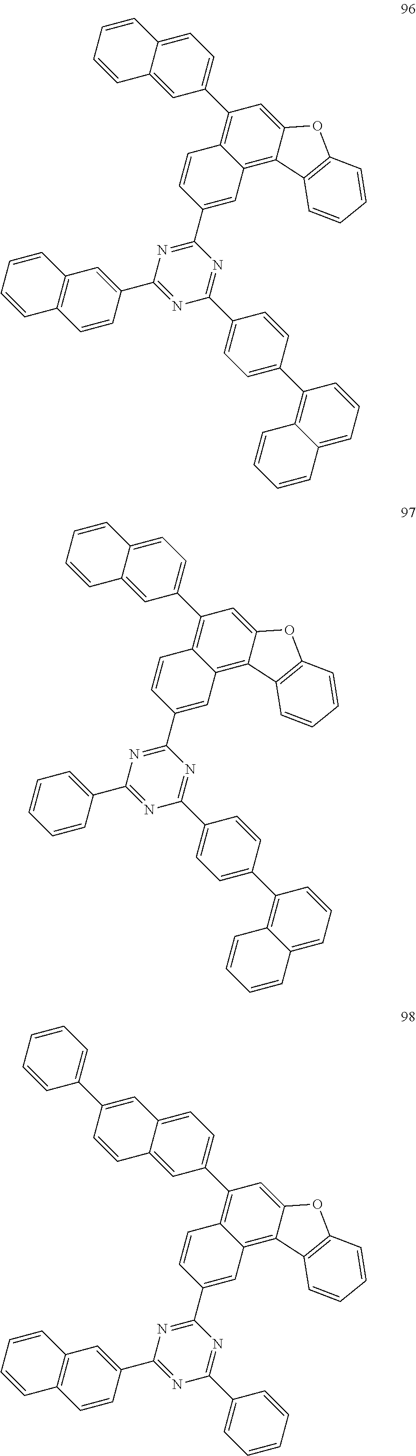 [SMILES: c1ccc2cc(-c3nc(-c4ccc(-c5cccc6ccccc56)cc4)nc(-c4ccc5c(-c6ccc7ccccc7c6)cc6oc7ccccc7c6c5c4)n3)ccc2c1 ; c1ccc(-c2nc(-c3ccc(-c4cccc5ccccc45)cc3)nc(-c3ccc4c(-c5ccc6ccccc6c5)cc5oc6ccccc6c5c4c3)n2)cc1 ; c1ccc(-c2ccc3cc(-c4cc5oc6ccccc6c5c5cc(-c6nc(-c7ccccc7)nc(-c7ccc8ccccc8c7)n6)ccc45)ccc3c2)cc1]

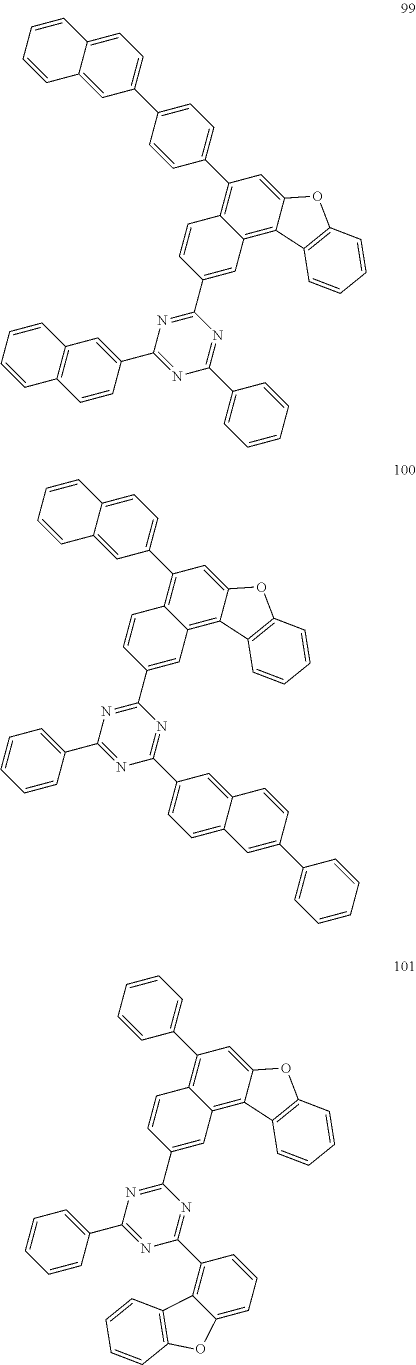 [SMILES: c1ccc(-c2nc(-c3ccc4ccccc4c3)nc(-c3ccc4c(-c5ccc(-c6ccc7ccccc7c6)cc5)cc5oc6ccccc6c5c4c3)n2)cc1 ; c1ccc(-c2ccc3cc(-c4nc(-c5ccccc5)nc(-c5ccc6c(-c7ccc8ccccc8c7)cc7oc8ccccc8c7c6c5)n4)ccc3c2)cc1 ; c1ccc(-c2nc(-c3ccc4c(-c5ccccc5)cc5oc6ccccc6c5c4c3)nc(-c3cccc4oc5ccccc5c34)n2)cc1]

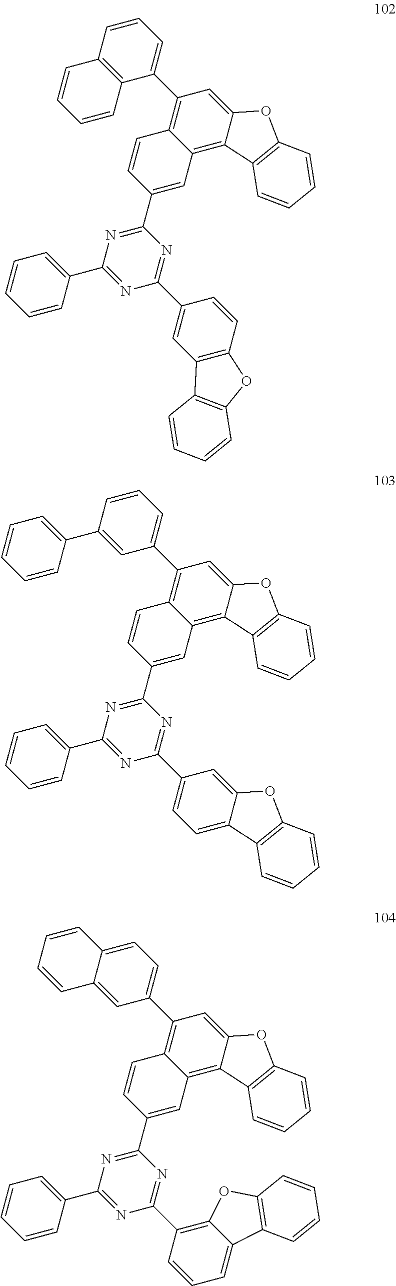 [SMILES: c1ccc(-c2nc(-c3ccc4oc5ccccc5c4c3)nc(-c3ccc4c(-c5cccc6ccccc56)cc5oc6ccccc6c5c4c3)n2)cc1 ; c1ccc(-c2cccc(-c3cc4oc5ccccc5c4c4cc(-c5nc(-c6ccccc6)nc(-c6ccc7c(c6)oc6ccccc67)n5)ccc34)c2)cc1 ; c1ccc(-c2nc(-c3ccc4c(-c5ccc6ccccc6c5)cc5oc6ccccc6c5c4c3)nc(-c3cccc4c3oc3ccccc34)n2)cc1]

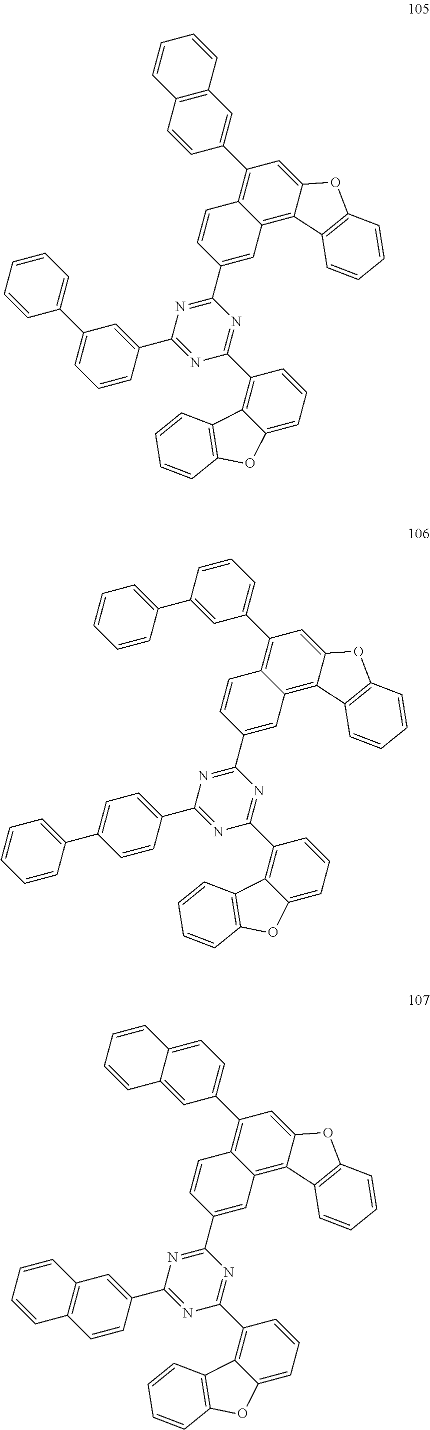 [SMILES: c1ccc(-c2cccc(-c3nc(-c4ccc5c(-c6ccc7ccccc7c6)cc6oc7ccccc7c6c5c4)nc(-c4cccc5oc6ccccc6c45)n3)c2)cc1 ; c1ccc(-c2ccc(-c3nc(-c4ccc5c(-c6cccc(-c7ccccc7)c6)cc6oc7ccccc7c6c5c4)nc(-c4cccc5oc6ccccc6c45)n3)cc2)cc1 ; c1ccc2cc(-c3nc(-c4ccc5c(-c6ccc7ccccc7c6)cc6oc7ccccc7c6c5c4)nc(-c4cccc5oc6ccccc6c45)n3)ccc2c1]

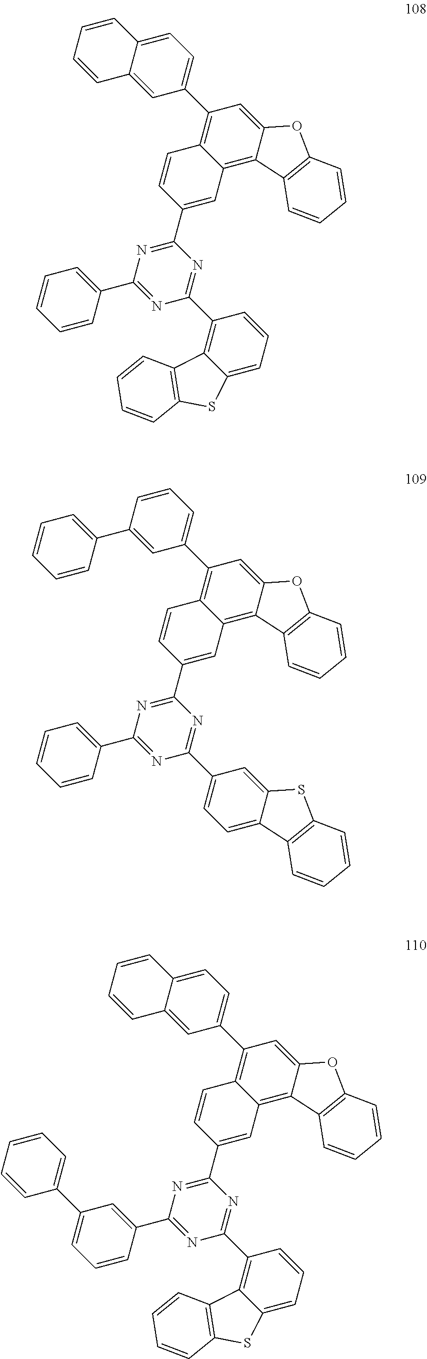 [SMILES: c1ccc(-c2nc(-c3ccc4c(-c5ccc6ccccc6c5)cc5oc6ccccc6c5c4c3)nc(-c3cccc4sc5ccccc5c34)n2)cc1 ; c1ccc(-c2cccc(-c3cc4oc5ccccc5c4c4cc(-c5nc(-c6ccccc6)nc(-c6ccc7c(c6)sc6ccccc67)n5)ccc34)c2)cc1 ; c1ccc(-c2cccc(-c3nc(-c4ccc5c(-c6ccc7ccccc7c6)cc6oc7ccccc7c6c5c4)nc(-c4cccc5sc6ccccc6c45)n3)c2)cc1]

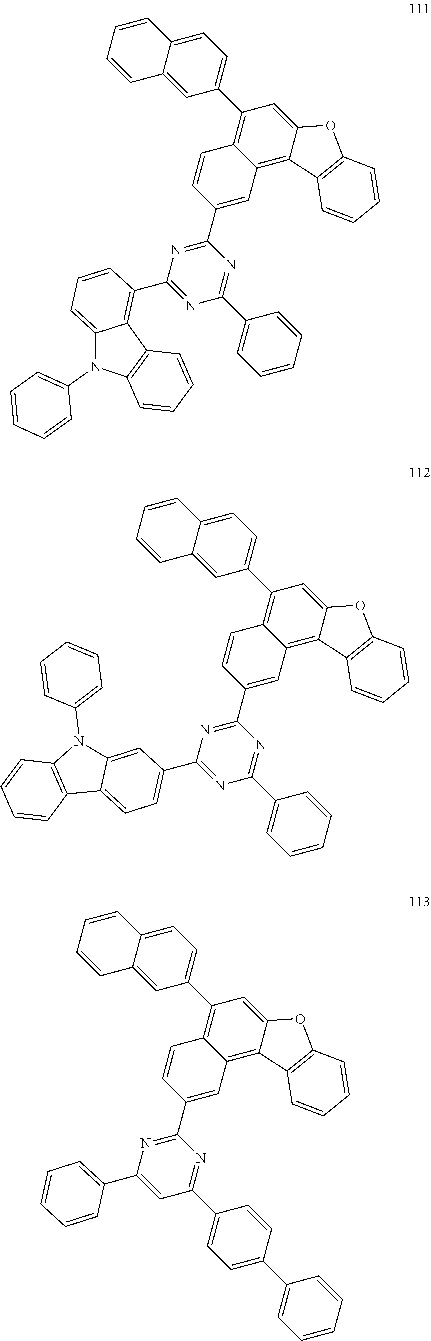 [SMILES: c1ccc(-c2nc(-c3ccc4c(-c5ccc6ccccc6c5)cc5oc6ccccc6c5c4c3)nc(-c3cccc4c3c3ccccc3n4-c3ccccc3)n2)cc1 ; c1ccc(-c2nc(-c3ccc4c(-c5ccc6ccccc6c5)cc5oc6ccccc6c5c4c3)nc(-c3ccc4c5ccccc5n(-c5ccccc5)c4c3)n2)cc1 ; c1ccc(-c2ccc(-c3cc(-c4ccccc4)nc(-c4ccc5c(-c6ccc7ccccc7c6)cc6oc7ccccc7c6c5c4)n3)cc2)cc1]

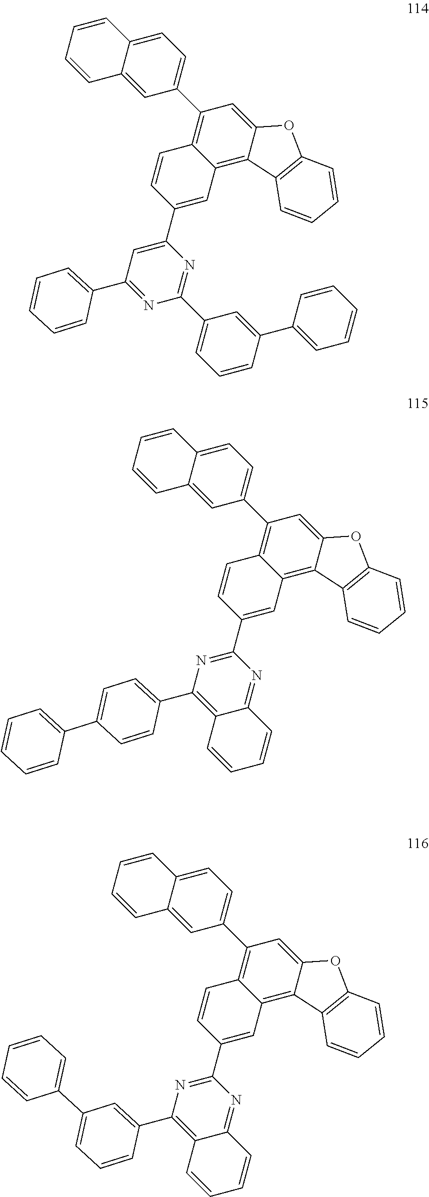 [SMILES: c1ccc(-c2cccc(-c3nc(-c4ccccc4)cc(-c4ccc5c(-c6ccc7ccccc7c6)cc6oc7ccccc7c6c5c4)n3)c2)cc1 ; c1ccc(-c2ccc(-c3nc(-c4ccc5c(-c6ccc7ccccc7c6)cc6oc7ccccc7c6c5c4)nc4ccccc34)cc2)cc1 ; c1ccc(-c2cccc(-c3nc(-c4ccc5c(-c6ccc7ccccc7c6)cc6oc7ccccc7c6c5c4)nc4ccccc34)c2)cc1]

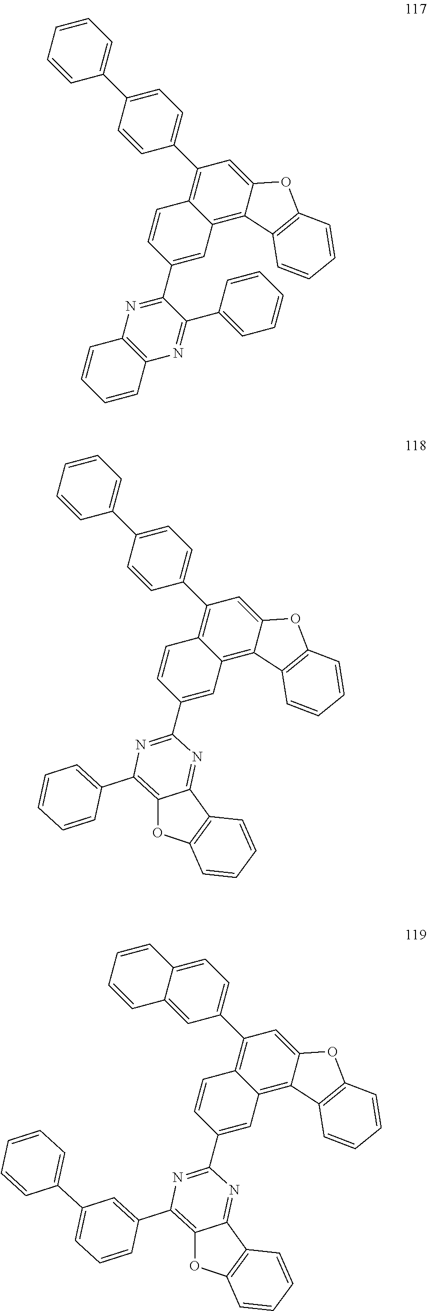 [SMILES: c1ccc(-c2ccc(-c3cc4oc5ccccc5c4c4cc(-c5nc6ccccc6nc5-c5ccccc5)ccc34)cc2)cc1 ; c1ccc(-c2ccc(-c3cc4oc5ccccc5c4c4cc(-c5nc(-c6ccccc6)c6oc7ccccc7c6n5)ccc34)cc2)cc1 ; c1ccc(-c2cccc(-c3nc(-c4ccc5c(-c6ccc7ccccc7c6)cc6oc7ccccc7c6c5c4)nc4c3oc3ccccc34)c2)cc1]

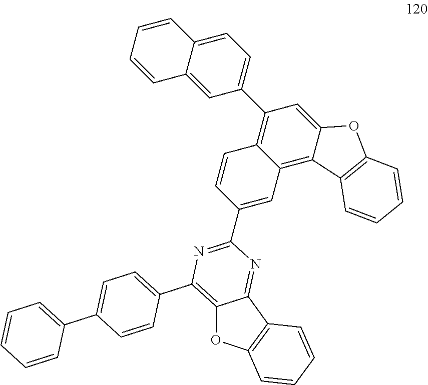 [SMILES: c1ccc(-c2ccc(-c3nc(-c4ccc5c(-c6ccc7ccccc7c6)cc6oc7ccccc7c6c5c4)nc4c3oc3ccccc34)cc2)cc1]

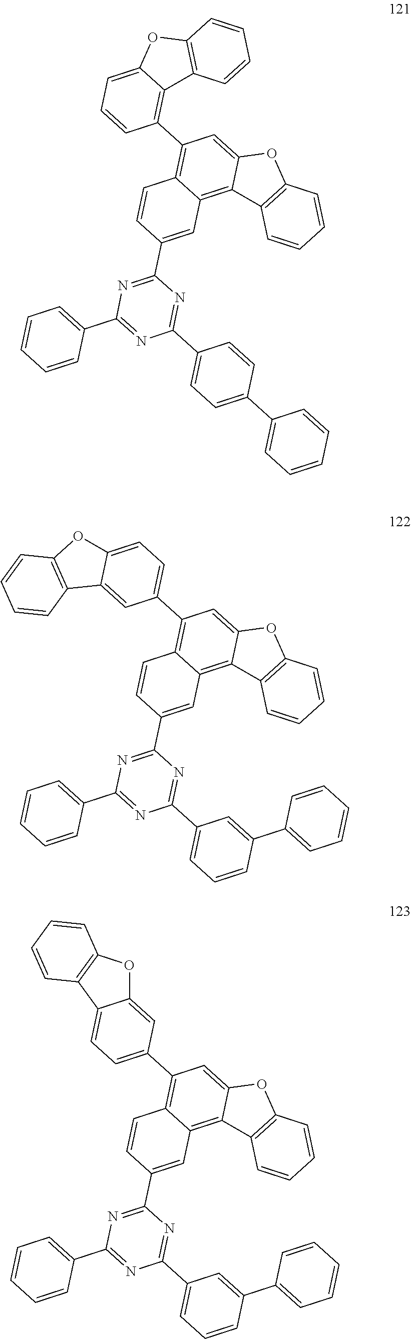 [SMILES: c1ccc(-c2ccc(-c3nc(-c4ccccc4)nc(-c4ccc5c(-c6cccc7oc8ccccc8c67)cc6oc7ccccc7c6c5c4)n3)cc2)cc1 ; c1ccc(-c2cccc(-c3nc(-c4ccccc4)nc(-c4ccc5c(-c6ccc7oc8ccccc8c7c6)cc6oc7ccccc7c6c5c4)n3)c2)cc1 ; c1ccc(-c2cccc(-c3nc(-c4ccccc4)nc(-c4ccc5c(-c6ccc7c(c6)oc6ccccc67)cc6oc7ccccc7c6c5c4)n3)c2)cc1]

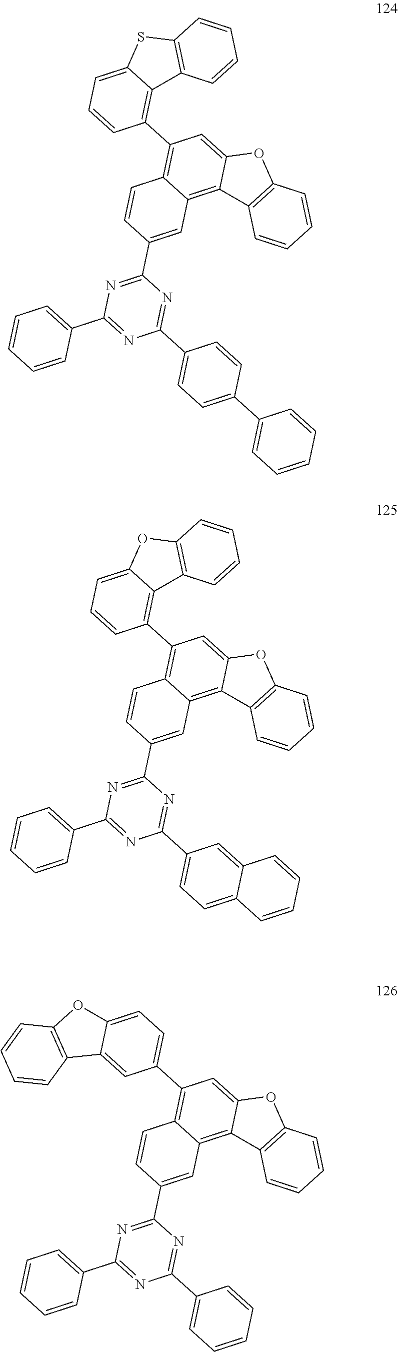 [SMILES: c1ccc(-c2ccc(-c3nc(-c4ccccc4)nc(-c4ccc5c(-c6cccc7sc8ccccc8c67)cc6oc7ccccc7c6c5c4)n3)cc2)cc1 ; c1ccc(-c2nc(-c3ccc4ccccc4c3)nc(-c3ccc4c(-c5cccc6oc7ccccc7c56)cc5oc6ccccc6c5c4c3)n2)cc1 ; c1ccc(-c2nc(-c3ccccc3)nc(-c3ccc4c(-c5ccc6oc7ccccc7c6c5)cc5oc6ccccc6c5c4c3)n2)cc1]

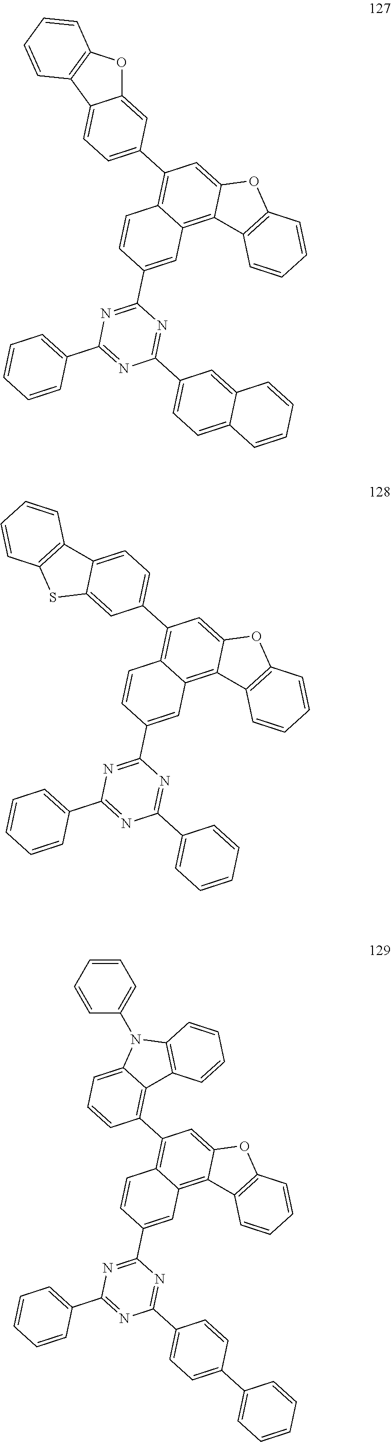 [SMILES: c1ccc(-c2nc(-c3ccc4ccccc4c3)nc(-c3ccc4c(-c5ccc6c(c5)oc5ccccc56)cc5oc6ccccc6c5c4c3)n2)cc1 ; c1ccc(-c2nc(-c3ccccc3)nc(-c3ccc4c(-c5ccc6c(c5)sc5ccccc56)cc5oc6ccccc6c5c4c3)n2)cc1 ; c1ccc(-c2ccc(-c3nc(-c4ccccc4)nc(-c4ccc5c(-c6cccc7c6c6ccccc6n7-c6ccccc6)cc6oc7ccccc7c6c5c4)n3)cc2)cc1]

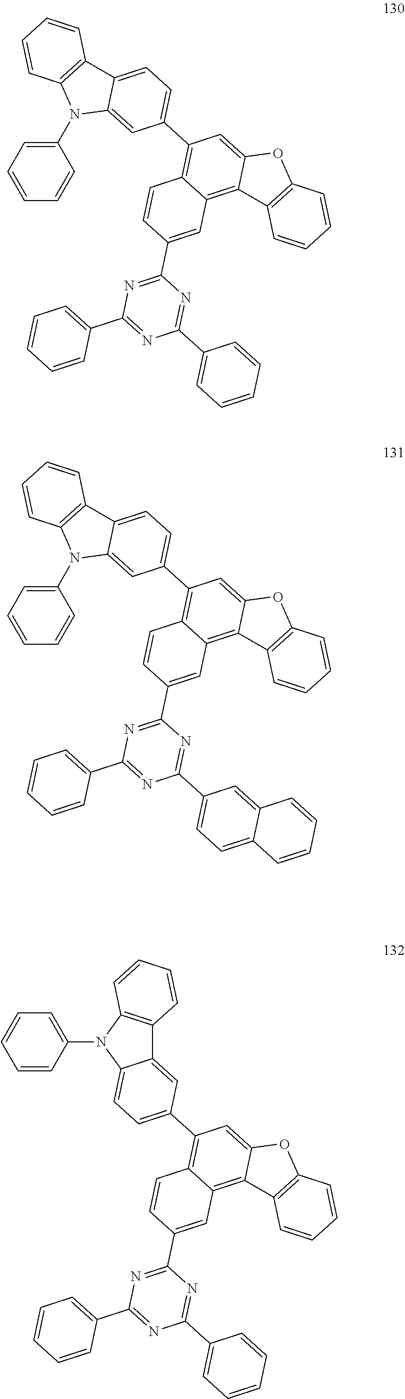 [SMILES: c1ccc(-c2nc(-c3ccccc3)nc(-c3ccc4c(-c5ccc6c7ccccc7n(-c7ccccc7)c6c5)cc5oc6ccccc6c5c4c3)n2)cc1 ; c1ccc(-c2nc(-c3ccc4ccccc4c3)nc(-c3ccc4c(-c5ccc6c7ccccc7n(-c7ccccc7)c6c5)cc5oc6ccccc6c5c4c3)n2)cc1 ; c1ccc(-c2nc(-c3ccccc3)nc(-c3ccc4c(-c5ccc6c(c5)c5ccccc5n6-c5ccccc5)cc5oc6ccccc6c5c4c3)n2)cc1]

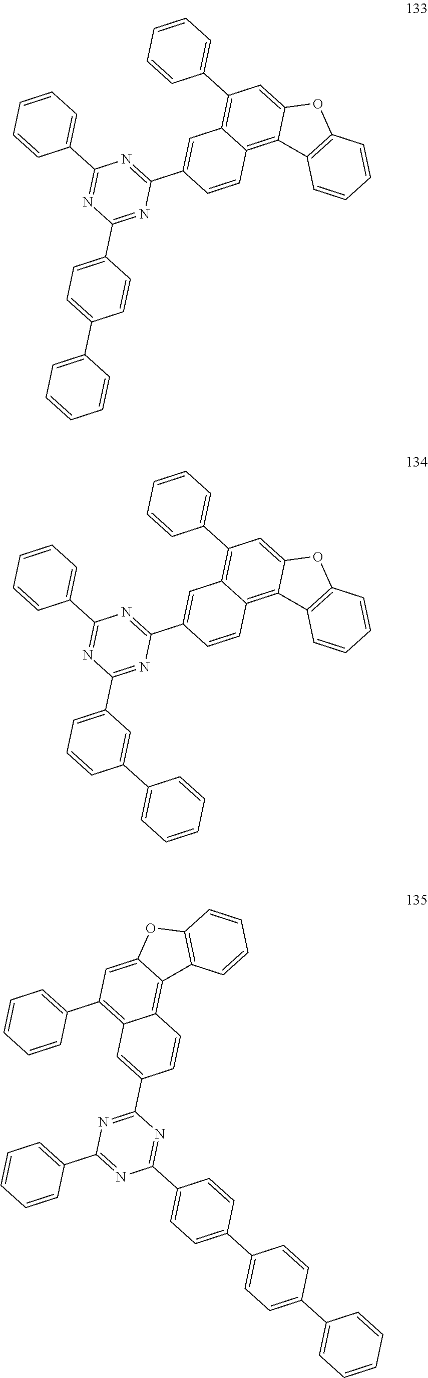 [SMILES: c1ccc(-c2ccc(-c3nc(-c4ccccc4)nc(-c4ccc5c(c4)c(-c4ccccc4)cc4oc6ccccc6c45)n3)cc2)cc1 ; c1ccc(-c2cccc(-c3nc(-c4ccccc4)nc(-c4ccc5c(c4)c(-c4ccccc4)cc4oc6ccccc6c45)n3)c2)cc1 ; c1ccc(-c2ccc(-c3ccc(-c4nc(-c5ccccc5)nc(-c5ccc6c(c5)c(-c5ccccc5)cc5oc7ccccc7c56)n4)cc3)cc2)cc1]

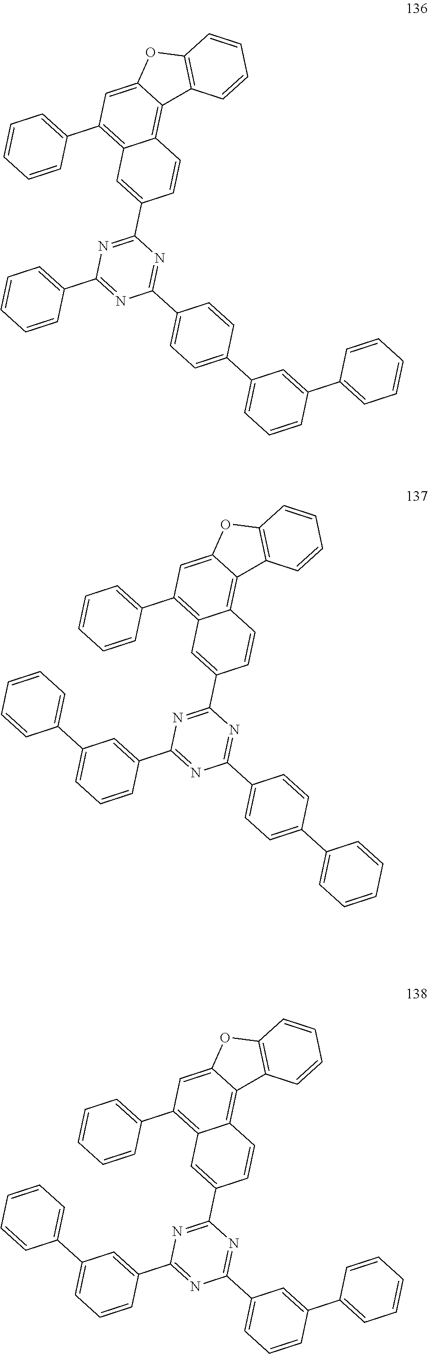 [SMILES: c1ccc(-c2cccc(-c3ccc(-c4nc(-c5ccccc5)nc(-c5ccc6c(c5)c(-c5ccccc5)cc5oc7ccccc7c56)n4)cc3)c2)cc1 ; c1ccc(-c2ccc(-c3nc(-c4cccc(-c5ccccc5)c4)nc(-c4ccc5c(c4)c(-c4ccccc4)cc4oc6ccccc6c45)n3)cc2)cc1 ; c1ccc(-c2cccc(-c3nc(-c4cccc(-c5ccccc5)c4)nc(-c4ccc5c(c4)c(-c4ccccc4)cc4oc6ccccc6c45)n3)c2)cc1]

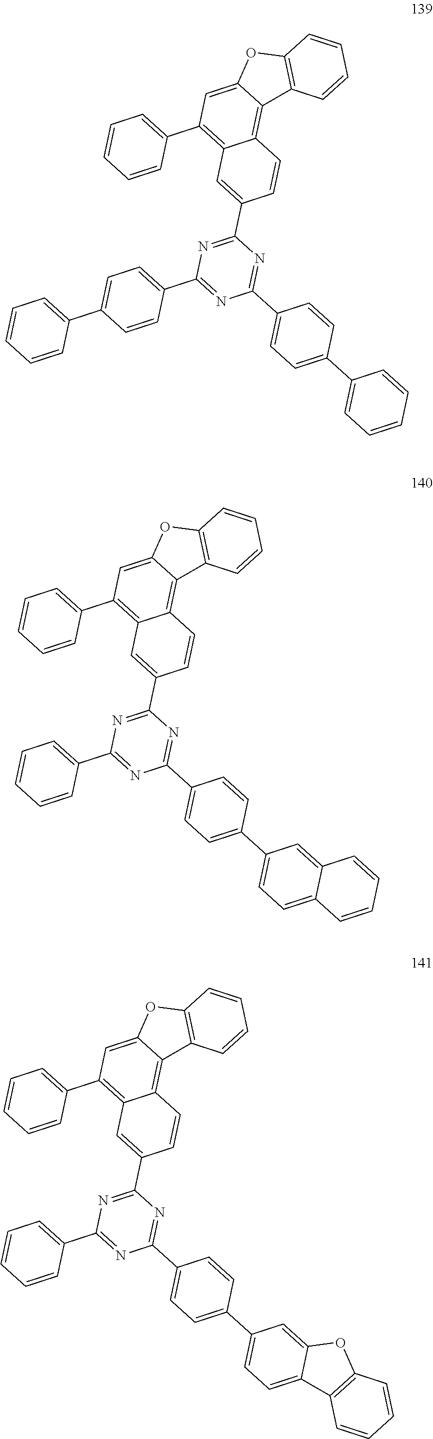 [SMILES: c1ccc(-c2ccc(-c3nc(-c4ccc(-c5ccccc5)cc4)nc(-c4ccc5c(c4)c(-c4ccccc4)cc4oc6ccccc6c45)n3)cc2)cc1 ; c1ccc(-c2nc(-c3ccc(-c4ccc5ccccc5c4)cc3)nc(-c3ccc4c(c3)c(-c3ccccc3)cc3oc5ccccc5c34)n2)cc1 ; c1ccc(-c2nc(-c3ccc(-c4ccc5c(c4)oc4ccccc45)cc3)nc(-c3ccc4c(c3)c(-c3ccccc3)cc3oc5ccccc5c34)n2)cc1]

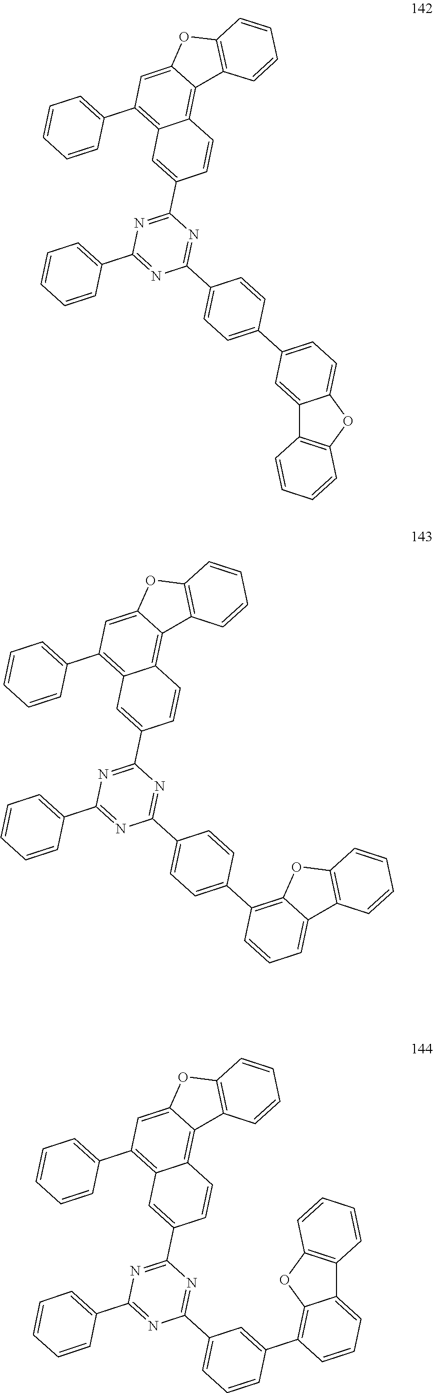 [SMILES: c1ccc(-c2nc(-c3ccc(-c4ccc5oc6ccccc6c5c4)cc3)nc(-c3ccc4c(c3)c(-c3ccccc3)cc3oc5ccccc5c34)n2)cc1 ; c1ccc(-c2nc(-c3ccc(-c4cccc5c4oc4ccccc45)cc3)nc(-c3ccc4c(c3)c(-c3ccccc3)cc3oc5ccccc5c34)n2)cc1 ; c1ccc(-c2nc(-c3cccc(-c4cccc5c4oc4ccccc45)c3)nc(-c3ccc4c(c3)c(-c3ccccc3)cc3oc5ccccc5c34)n2)cc1]

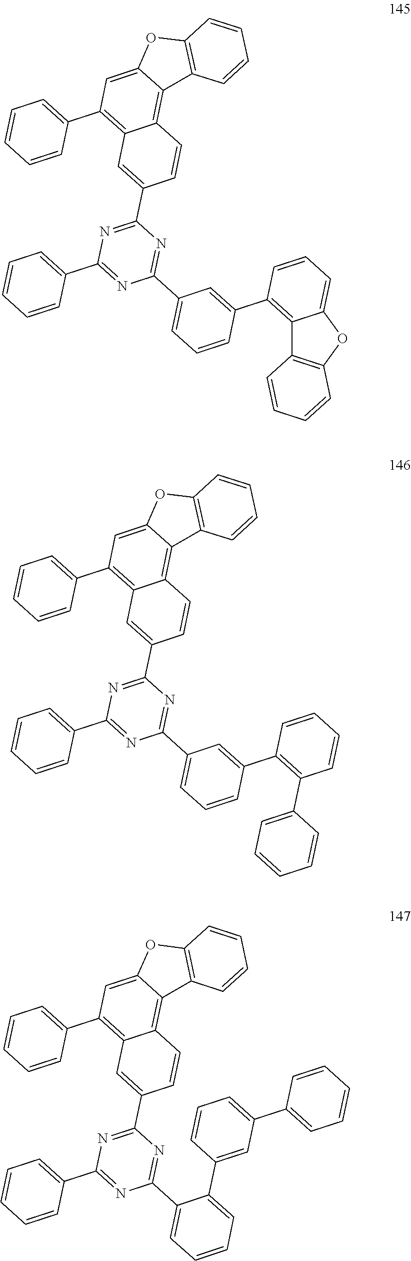 [SMILES: c1ccc(-c2nc(-c3cccc(-c4cccc5oc6ccccc6c45)c3)nc(-c3ccc4c(c3)c(-c3ccccc3)cc3oc5ccccc5c34)n2)cc1 ; c1ccc(-c2nc(-c3cccc(-c4ccccc4-c4ccccc4)c3)nc(-c3ccc4c(c3)c(-c3ccccc3)cc3oc5ccccc5c34)n2)cc1 ; c1ccc(-c2cccc(-c3ccccc3-c3nc(-c4ccccc4)nc(-c4ccc5c(c4)c(-c4ccccc4)cc4oc6ccccc6c45)n3)c2)cc1]

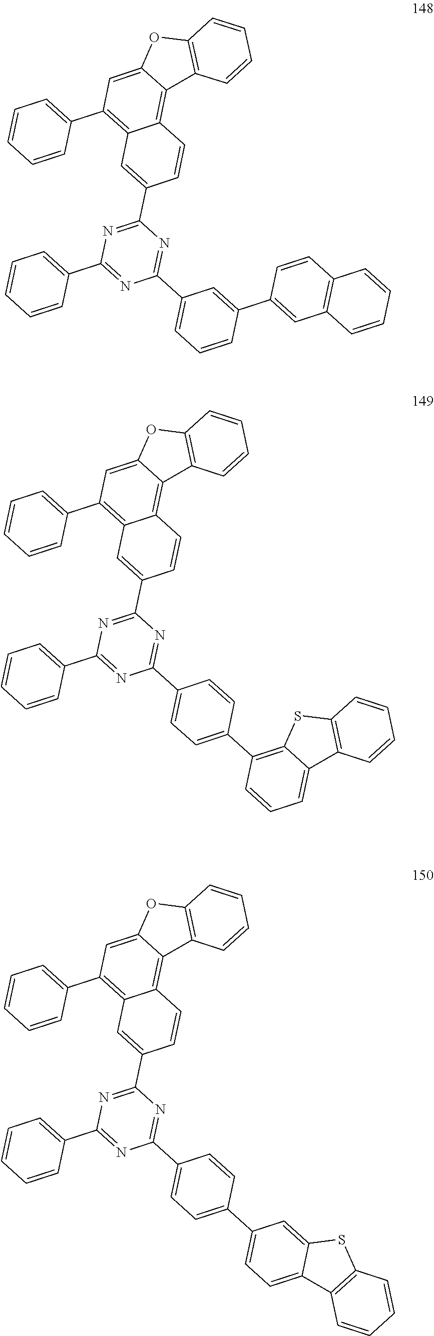 [SMILES: c1ccc(-c2nc(-c3cccc(-c4ccc5ccccc5c4)c3)nc(-c3ccc4c(c3)c(-c3ccccc3)cc3oc5ccccc5c34)n2)cc1 ; c1ccc(-c2nc(-c3ccc(-c4cccc5c4sc4ccccc45)cc3)nc(-c3ccc4c(c3)c(-c3ccccc3)cc3oc5ccccc5c34)n2)cc1 ; c1ccc(-c2nc(-c3ccc(-c4ccc5c(c4)sc4ccccc45)cc3)nc(-c3ccc4c(c3)c(-c3ccccc3)cc3oc5ccccc5c34)n2)cc1]

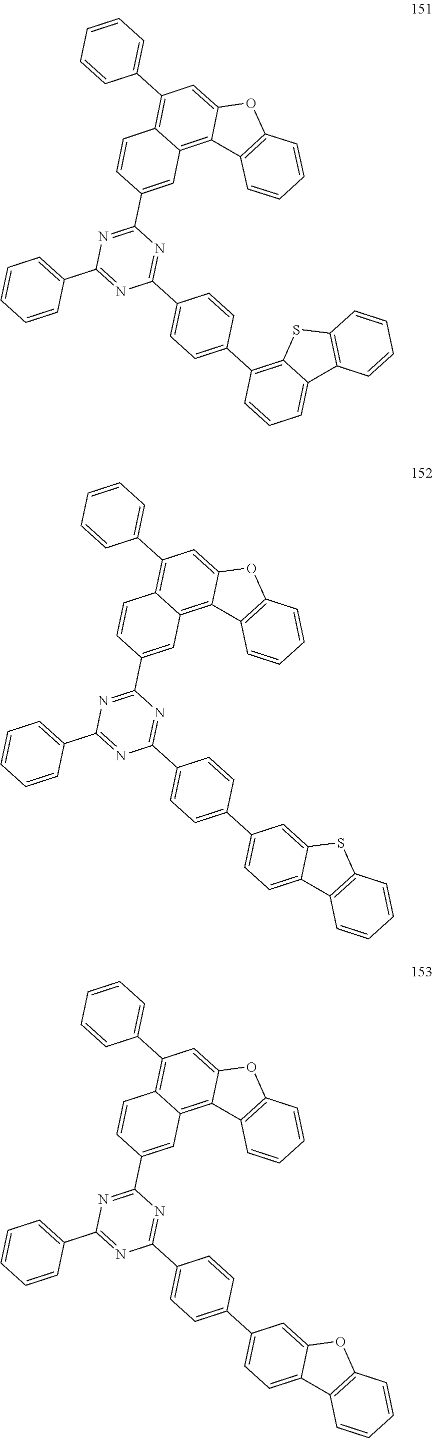 [SMILES: c1ccc(-c2nc(-c3ccc(-c4cccc5c4sc4ccccc45)cc3)nc(-c3ccc4c(-c5ccccc5)cc5oc6ccccc6c5c4c3)n2)cc1 ; c1ccc(-c2nc(-c3ccc(-c4ccc5c(c4)sc4ccccc45)cc3)nc(-c3ccc4c(-c5ccccc5)cc5oc6ccccc6c5c4c3)n2)cc1 ; c1ccc(-c2nc(-c3ccc(-c4ccc5c(c4)oc4ccccc45)cc3)nc(-c3ccc4c(-c5ccccc5)cc5oc6ccccc6c5c4c3)n2)cc1]

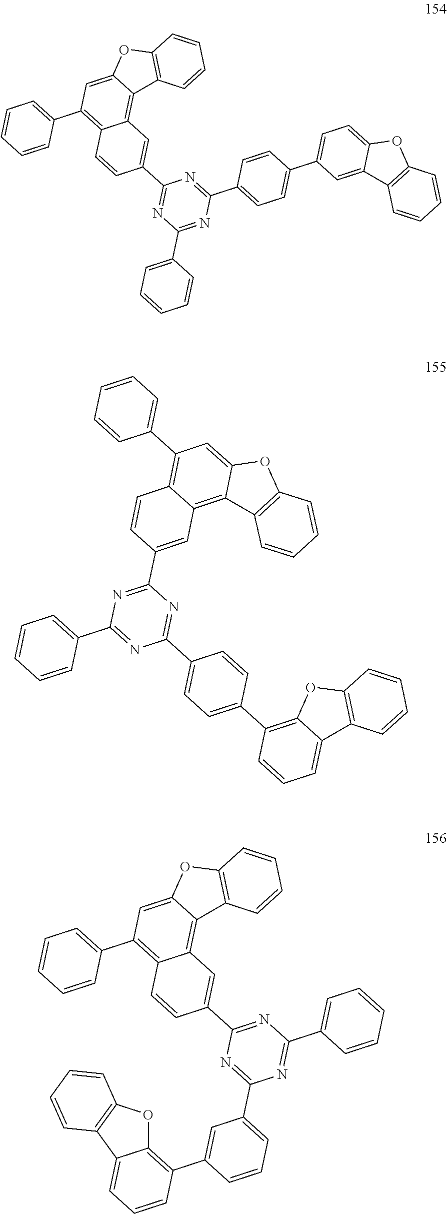 [SMILES: c1ccc(-c2nc(-c3ccc(-c4ccc5oc6ccccc6c5c4)cc3)nc(-c3ccc4c(-c5ccccc5)cc5oc6ccccc6c5c4c3)n2)cc1 ; c1ccc(-c2nc(-c3ccc(-c4cccc5c4oc4ccccc45)cc3)nc(-c3ccc4c(-c5ccccc5)cc5oc6ccccc6c5c4c3)n2)cc1 ; c1ccc(-c2nc(-c3cccc(-c4cccc5c4oc4ccccc45)c3)nc(-c3ccc4c(-c5ccccc5)cc5oc6ccccc6c5c4c3)n2)cc1]

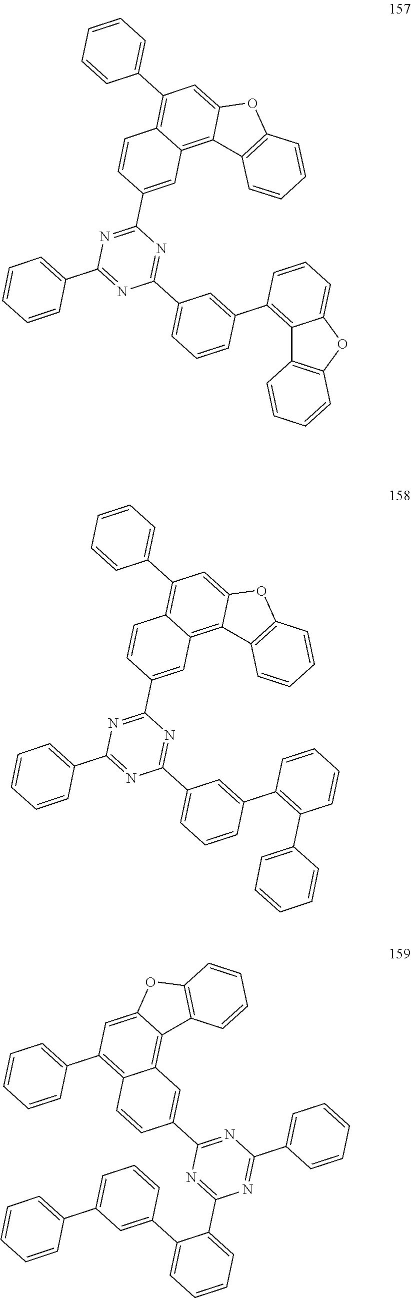 [SMILES: c1ccc(-c2nc(-c3cccc(-c4cccc5oc6ccccc6c45)c3)nc(-c3ccc4c(-c5ccccc5)cc5oc6ccccc6c5c4c3)n2)cc1 ; c1ccc(-c2nc(-c3cccc(-c4ccccc4-c4ccccc4)c3)nc(-c3ccc4c(-c5ccccc5)cc5oc6ccccc6c5c4c3)n2)cc1 ; c1ccc(-c2cccc(-c3ccccc3-c3nc(-c4ccccc4)nc(-c4ccc5c(-c6ccccc6)cc6oc7ccccc7c6c5c4)n3)c2)cc1]

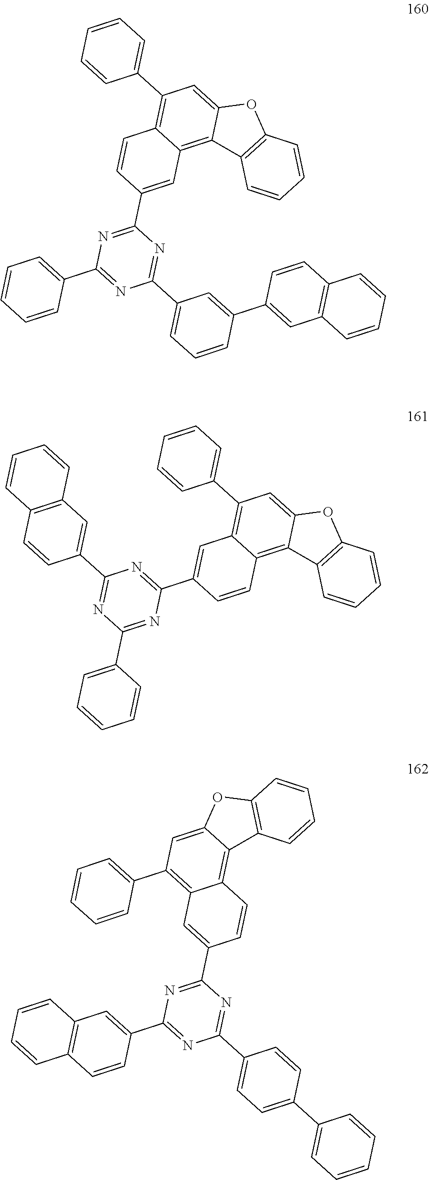 [SMILES: c1ccc(-c2nc(-c3cccc(-c4ccc5ccccc5c4)c3)nc(-c3ccc4c(-c5ccccc5)cc5oc6ccccc6c5c4c3)n2)cc1 ; c1ccc(-c2nc(-c3ccc4ccccc4c3)nc(-c3ccc4c(c3)c(-c3ccccc3)cc3oc5ccccc5c34)n2)cc1 ; c1ccc(-c2ccc(-c3nc(-c4ccc5ccccc5c4)nc(-c4ccc5c(c4)c(-c4ccccc4)cc4oc6ccccc6c45)n3)cc2)cc1]

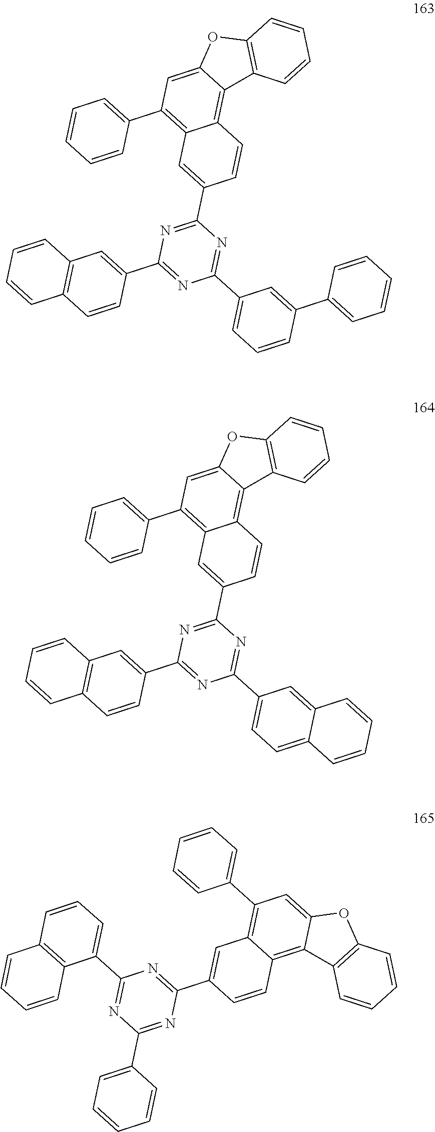 [SMILES: c1ccc(-c2cccc(-c3nc(-c4ccc5ccccc5c4)nc(-c4ccc5c(c4)c(-c4ccccc4)cc4oc6ccccc6c45)n3)c2)cc1 ; c1ccc(-c2cc3oc4ccccc4c3c3ccc(-c4nc(-c5ccc6ccccc6c5)nc(-c5ccc6ccccc6c5)n4)cc23)cc1 ; c1ccc(-c2nc(-c3ccc4c(c3)c(-c3ccccc3)cc3oc5ccccc5c34)nc(-c3cccc4ccccc34)n2)cc1]

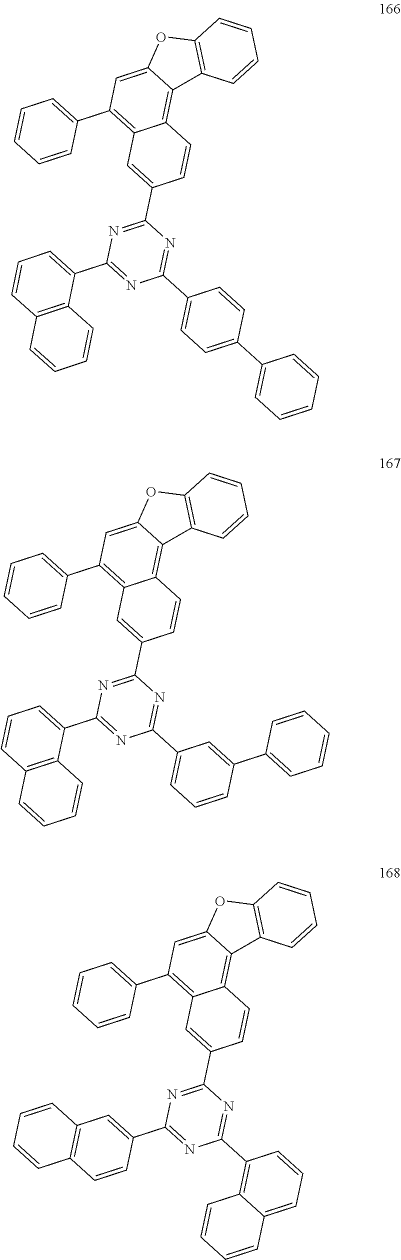 [SMILES: c1ccc(-c2ccc(-c3nc(-c4ccc5c(c4)c(-c4ccccc4)cc4oc6ccccc6c45)nc(-c4cccc5ccccc45)n3)cc2)cc1 ; c1ccc(-c2cccc(-c3nc(-c4ccc5c(c4)c(-c4ccccc4)cc4oc6ccccc6c45)nc(-c4cccc5ccccc45)n3)c2)cc1 ; c1ccc(-c2cc3oc4ccccc4c3c3ccc(-c4nc(-c5ccc6ccccc6c5)nc(-c5cccc6ccccc56)n4)cc23)cc1]

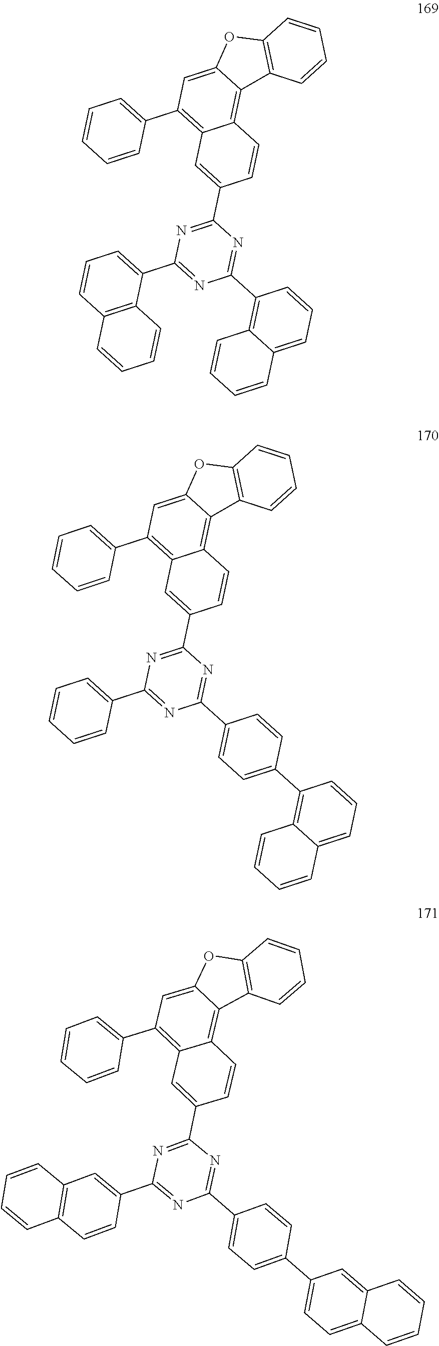 [SMILES: c1ccc(-c2cc3oc4ccccc4c3c3ccc(-c4nc(-c5cccc6ccccc56)nc(-c5cccc6ccccc56)n4)cc23)cc1 ; c1ccc(-c2nc(-c3ccc(-c4cccc5ccccc45)cc3)nc(-c3ccc4c(c3)c(-c3ccccc3)cc3oc5ccccc5c34)n2)cc1 ; c1ccc(-c2cc3oc4ccccc4c3c3ccc(-c4nc(-c5ccc(-c6ccc7ccccc7c6)cc5)nc(-c5ccc6ccccc6c5)n4)cc23)cc1]

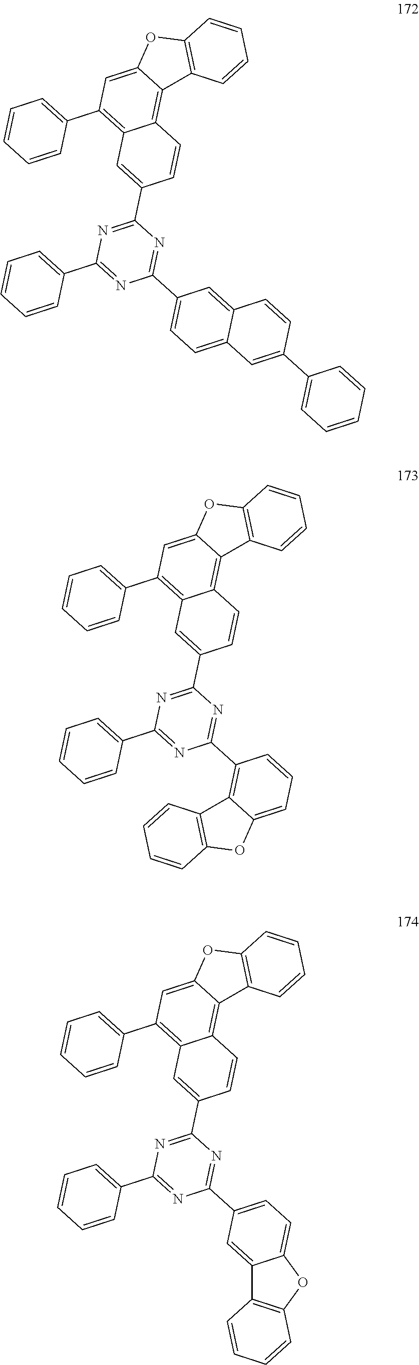 [SMILES: c1ccc(-c2ccc3cc(-c4nc(-c5ccccc5)nc(-c5ccc6c(c5)c(-c5ccccc5)cc5oc7ccccc7c56)n4)ccc3c2)cc1 ; c1ccc(-c2nc(-c3ccc4c(c3)c(-c3ccccc3)cc3oc5ccccc5c34)nc(-c3cccc4oc5ccccc5c34)n2)cc1 ; c1ccc(-c2nc(-c3ccc4c(c3)c(-c3ccccc3)cc3oc5ccccc5c34)nc(-c3ccc4oc5ccccc5c4c3)n2)cc1]

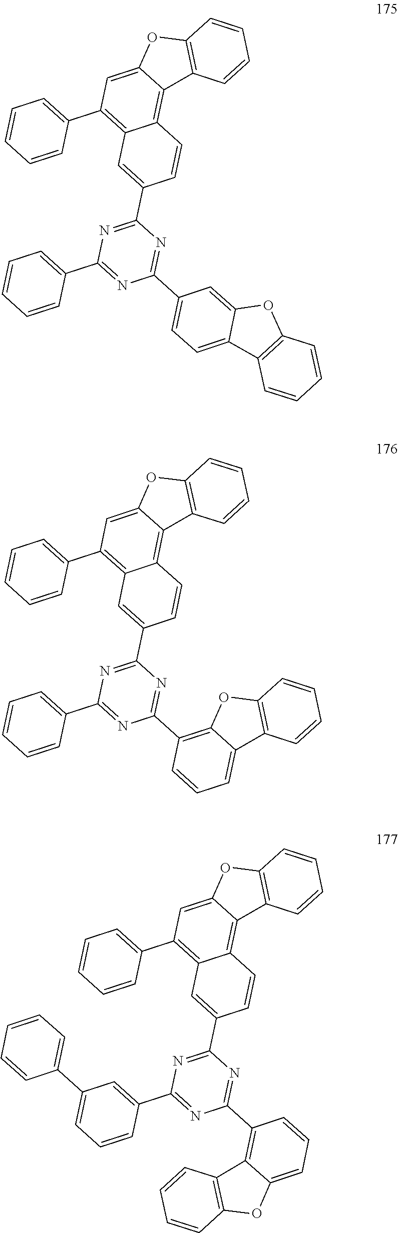 [SMILES: c1ccc(-c2nc(-c3ccc4c(c3)oc3ccccc34)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3cccc4c3oc3ccccc34)n2)cc1 ; c1ccc(-c2cccc(-c3nc(-c4ccc5c(c4)c(-c4ccccc4)cc4oc6ccccc6c45)nc(-c4cccc5oc6ccccc6c45)n3)c2)cc1]

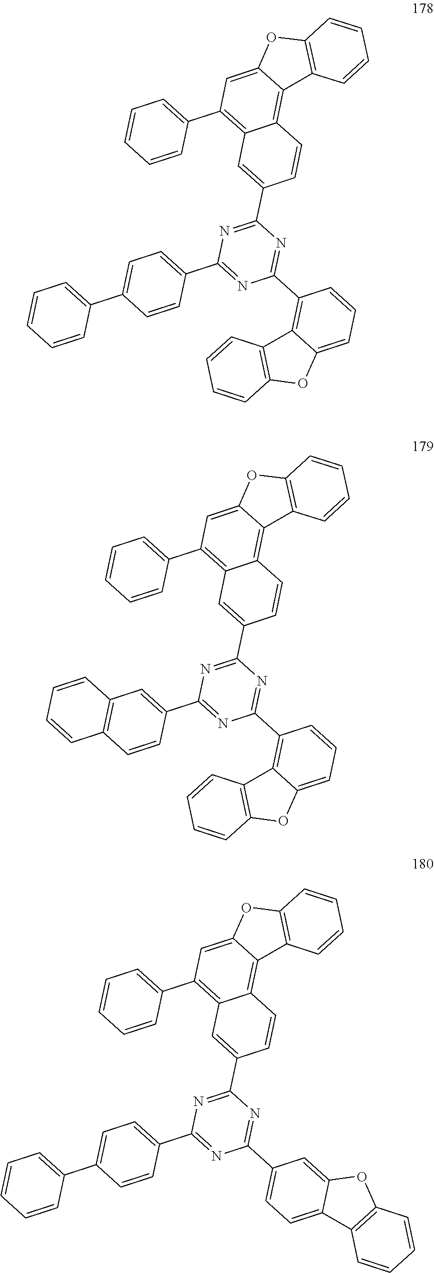 [SMILES: c1ccc(-c2ccc(-c3nc(-c4ccc5c(c4)c(-c4ccccc4)cc4oc6ccccc6c45)nc(-c4cccc5oc6ccccc6c45)n3)cc2)cc1 ; c1ccc(-c2cc3oc4ccccc4c3c3ccc(-c4nc(-c5ccc6ccccc6c5)nc(-c5cccc6oc7ccccc7c56)n4)cc23)cc1 ; c1ccc(-c2ccc(-c3nc(-c4ccc5c(c4)oc4ccccc45)nc(-c4ccc5c(c4)c(-c4ccccc4)cc4oc6ccccc6c45)n3)cc2)cc1]

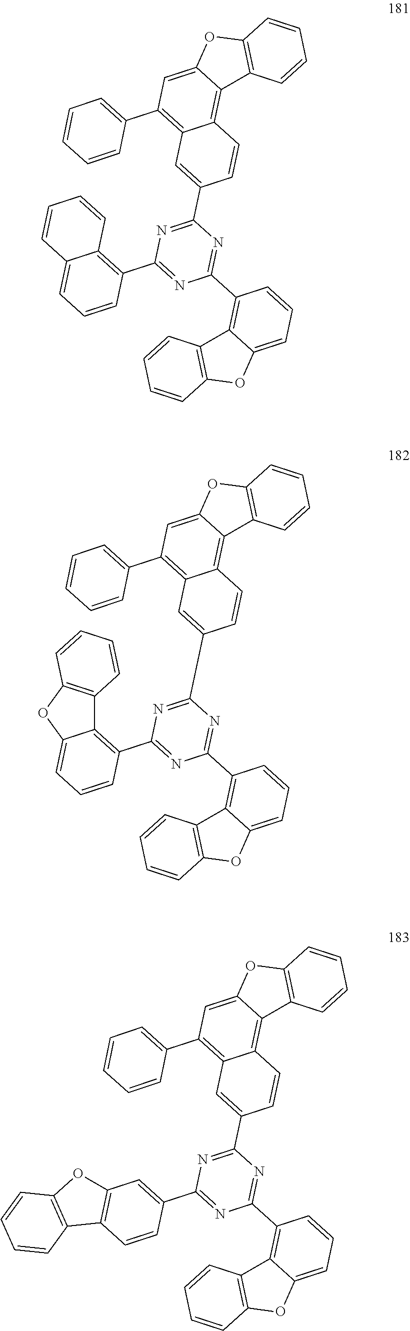 [SMILES: c1ccc(-c2cc3oc4ccccc4c3c3ccc(-c4nc(-c5cccc6ccccc56)nc(-c5cccc6oc7ccccc7c56)n4)cc23)cc1 ; c1ccc(-c2cc3oc4ccccc4c3c3ccc(-c4nc(-c5cccc6oc7ccccc7c56)nc(-c5cccc6oc7ccccc7c56)n4)cc23)cc1 ; c1ccc(-c2cc3oc4ccccc4c3c3ccc(-c4nc(-c5ccc6c(c5)oc5ccccc56)nc(-c5cccc6oc7ccccc7c56)n4)cc23)cc1]

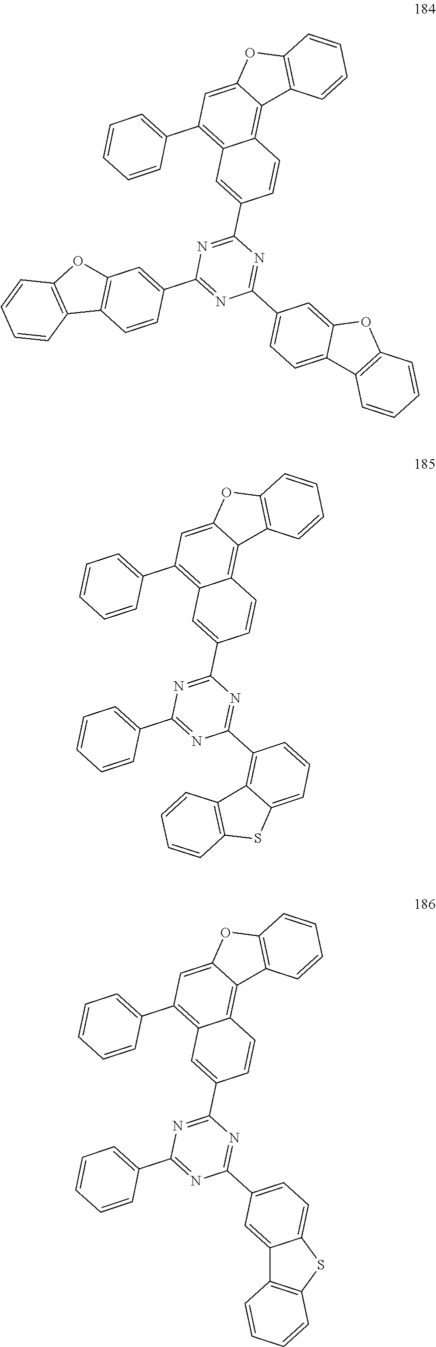 [SMILES: c1ccc(-c2cc3oc4ccccc4c3c3ccc(-c4nc(-c5ccc6c(c5)oc5ccccc56)nc(-c5ccc6c(c5)oc5ccccc56)n4)cc23)cc1 ; c1ccc(-c2nc(-c3ccc4c(c3)c(-c3ccccc3)cc3oc5ccccc5c34)nc(-c3cccc4sc5ccccc5c34)n2)cc1 ; c1ccc(-c2nc(-c3ccc4c(c3)c(-c3ccccc3)cc3oc5ccccc5c34)nc(-c3ccc4sc5ccccc5c4c3)n2)cc1]

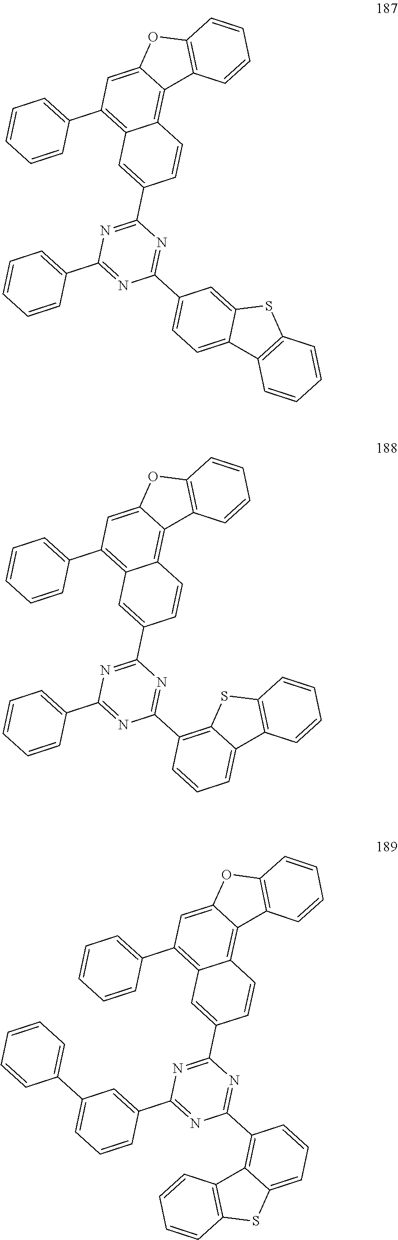 [SMILES: c1ccc(-c2nc(-c3ccc4c(c3)sc3ccccc34)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3cccc4c3sc3ccccc34)n2)cc1 ; c1ccc(-c2cccc(-c3nc(-c4ccc5c(c4)c(-c4ccccc4)cc4oc6ccccc6c45)nc(-c4cccc5sc6ccccc6c45)n3)c2)cc1]

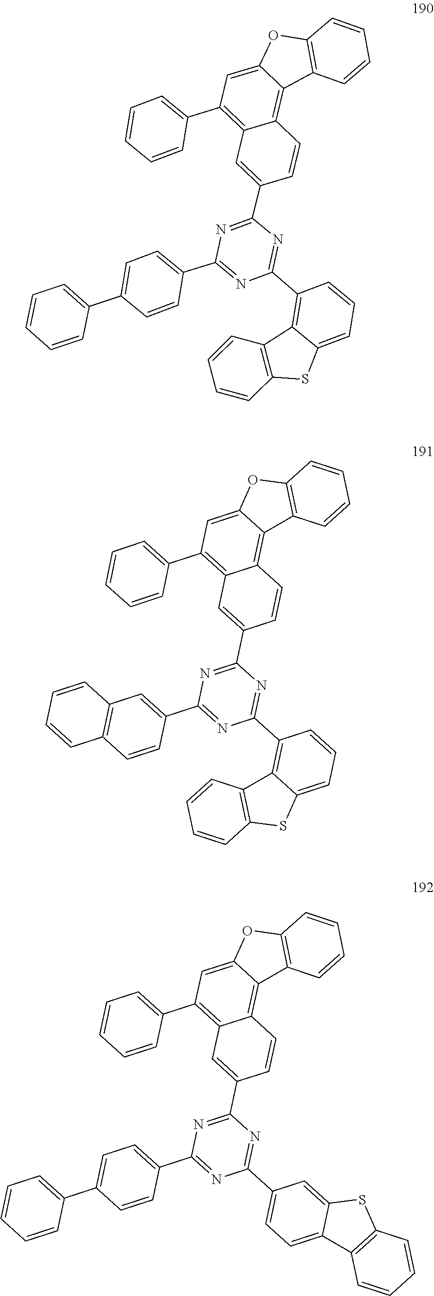 [SMILES: c1ccc(-c2ccc(-c3nc(-c4ccc5c(c4)c(-c4ccccc4)cc4oc6ccccc6c45)nc(-c4cccc5sc6ccccc6c45)n3)cc2)cc1 ; c1ccc(-c2cc3oc4ccccc4c3c3ccc(-c4nc(-c5ccc6ccccc6c5)nc(-c5cccc6sc7ccccc7c56)n4)cc23)cc1 ; c1ccc(-c2ccc(-c3nc(-c4ccc5c(c4)sc4ccccc45)nc(-c4ccc5c(c4)c(-c4ccccc4)cc4oc6ccccc6c45)n3)cc2)cc1]

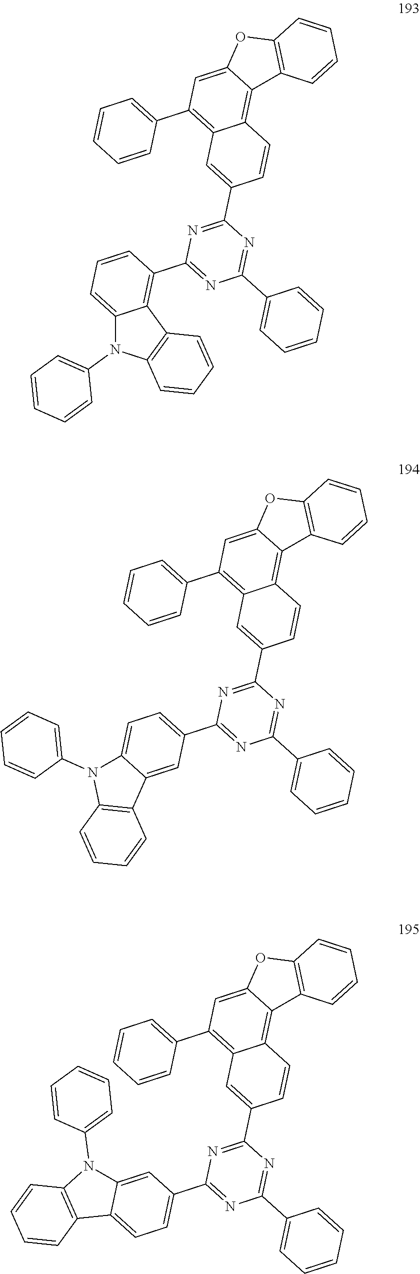 [SMILES: c1ccc(-c2nc(-c3ccc4c(c3)c(-c3ccccc3)cc3oc5ccccc5c34)nc(-c3cccc4c3c3ccccc3n4-c3ccccc3)n2)cc1 ; c1ccc(-c2nc(-c3ccc4c(c3)c(-c3ccccc3)cc3oc5ccccc5c34)nc(-c3ccc4c(c3)c3ccccc3n4-c3ccccc3)n2)cc1 ; c1ccc(-c2nc(-c3ccc4c(c3)c(-c3ccccc3)cc3oc5ccccc5c34)nc(-c3ccc4c5ccccc5n(-c5ccccc5)c4c3)n2)cc1]

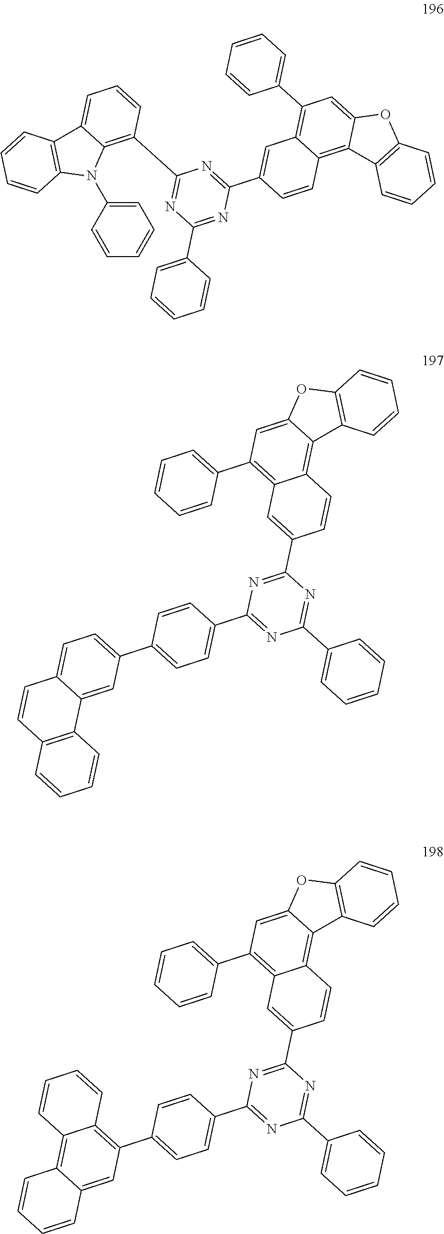 [SMILES: c1ccc(-c2nc(-c3ccc4c(c3)c(-c3ccccc3)cc3oc5ccccc5c34)nc(-c3cccc4c5ccccc5n(-c5ccccc5)c34)n2)cc1 ; c1ccc(-c2nc(-c3ccc(-c4ccc5ccc6ccccc6c5c4)cc3)nc(-c3ccc4c(c3)c(-c3ccccc3)cc3oc5ccccc5c34)n2)cc1 ; c1ccc(-c2nc(-c3ccc(-c4cc5ccccc5c5ccccc45)cc3)nc(-c3ccc4c(c3)c(-c3ccccc3)cc3oc5ccccc5c34)n2)cc1]

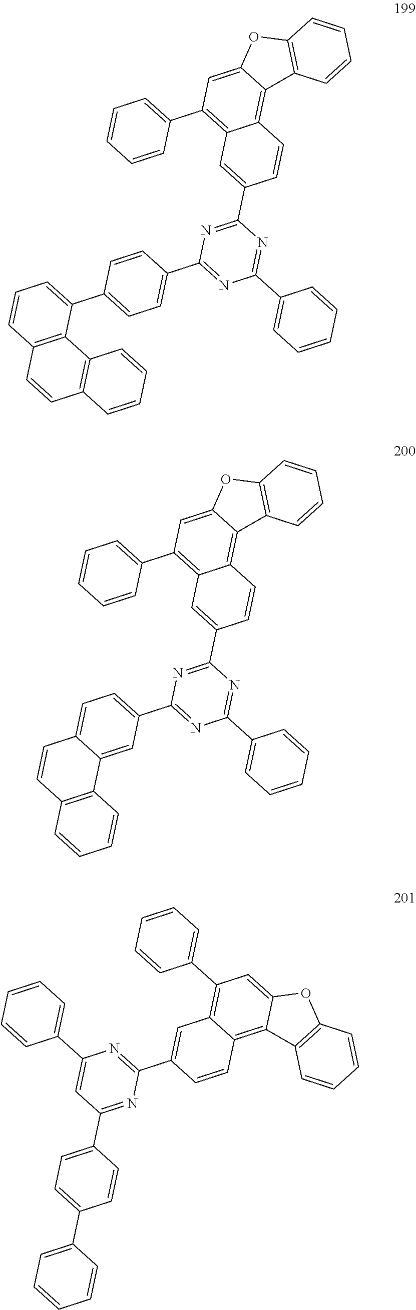 [SMILES: c1ccc(-c2nc(-c3ccc(-c4cccc5ccc6ccccc6c45)cc3)nc(-c3ccc4c(c3)c(-c3ccccc3)cc3oc5ccccc5c34)n2)cc1 ; c1ccc(-c2nc(-c3ccc4c(c3)c(-c3ccccc3)cc3oc5ccccc5c34)nc(-c3ccc4ccc5ccccc5c4c3)n2)cc1 ; c1ccc(-c2ccc(-c3cc(-c4ccccc4)nc(-c4ccc5c(c4)c(-c4ccccc4)cc4oc6ccccc6c45)n3)cc2)cc1]

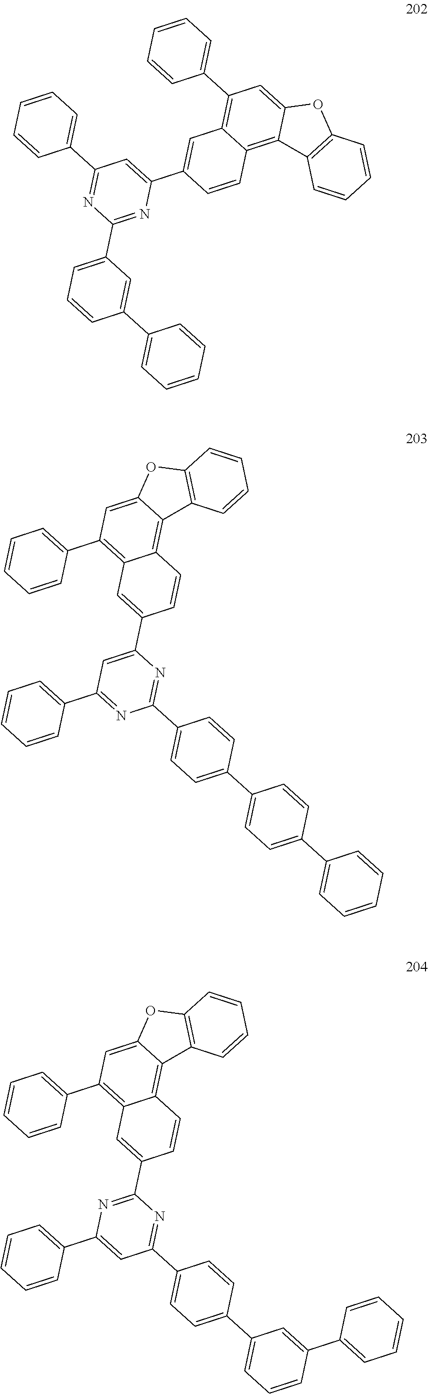 [SMILES: c1ccc(-c2cccc(-c3nc(-c4ccccc4)cc(-c4ccc5c(c4)c(-c4ccccc4)cc4oc6ccccc6c45)n3)c2)cc1 ; c1ccc(-c2ccc(-c3ccc(-c4nc(-c5ccccc5)cc(-c5ccc6c(c5)c(-c5ccccc5)cc5oc7ccccc7c56)n4)cc3)cc2)cc1 ; c1ccc(-c2cccc(-c3ccc(-c4cc(-c5ccccc5)nc(-c5ccc6c(c5)c(-c5ccccc5)cc5oc7ccccc7c56)n4)cc3)c2)cc1]

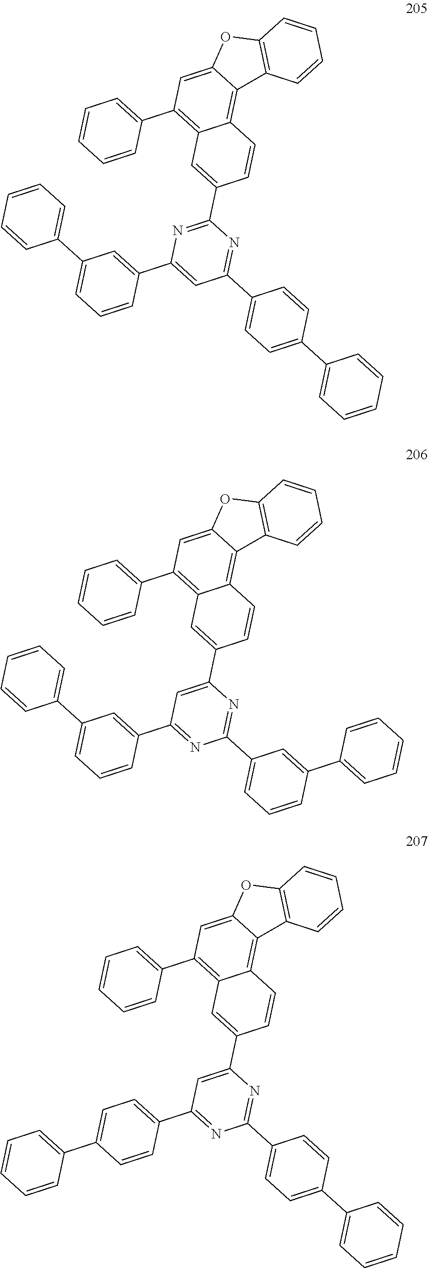 [SMILES: c1ccc(-c2ccc(-c3cc(-c4cccc(-c5ccccc5)c4)nc(-c4ccc5c(c4)c(-c4ccccc4)cc4oc6ccccc6c45)n3)cc2)cc1 ; c1ccc(-c2cccc(-c3cc(-c4ccc5c(c4)c(-c4ccccc4)cc4oc6ccccc6c45)nc(-c4cccc(-c5ccccc5)c4)n3)c2)cc1 ; c1ccc(-c2ccc(-c3cc(-c4ccc5c(c4)c(-c4ccccc4)cc4oc6ccccc6c45)nc(-c4ccc(-c5ccccc5)cc4)n3)cc2)cc1]

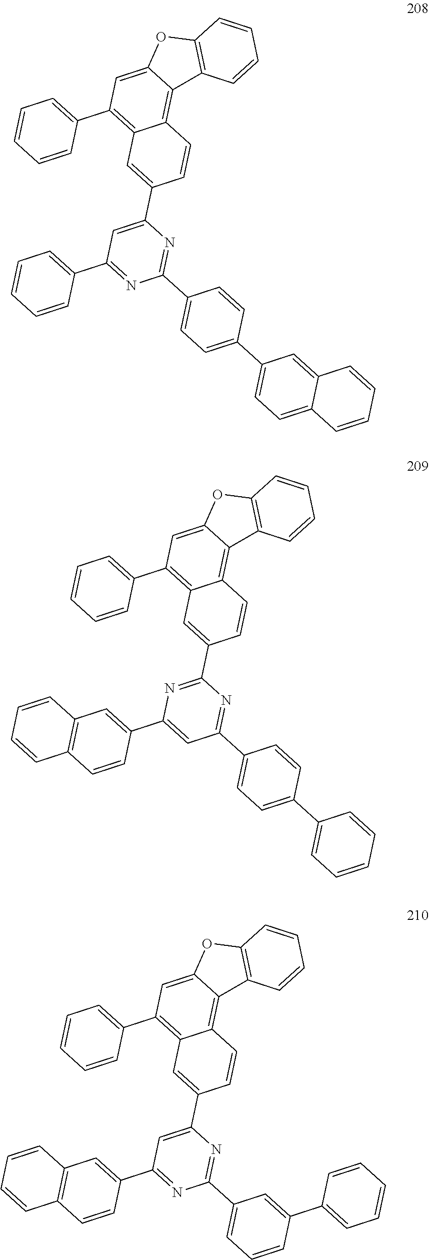 [SMILES: c1ccc(-c2cc(-c3ccc4c(c3)c(-c3ccccc3)cc3oc5ccccc5c34)nc(-c3ccc(-c4ccc5ccccc5c4)cc3)n2)cc1 ; c1ccc(-c2ccc(-c3cc(-c4ccc5ccccc5c4)nc(-c4ccc5c(c4)c(-c4ccccc4)cc4oc6ccccc6c45)n3)cc2)cc1 ; c1ccc(-c2cccc(-c3nc(-c4ccc5ccccc5c4)cc(-c4ccc5c(c4)c(-c4ccccc4)cc4oc6ccccc6c45)n3)c2)cc1]

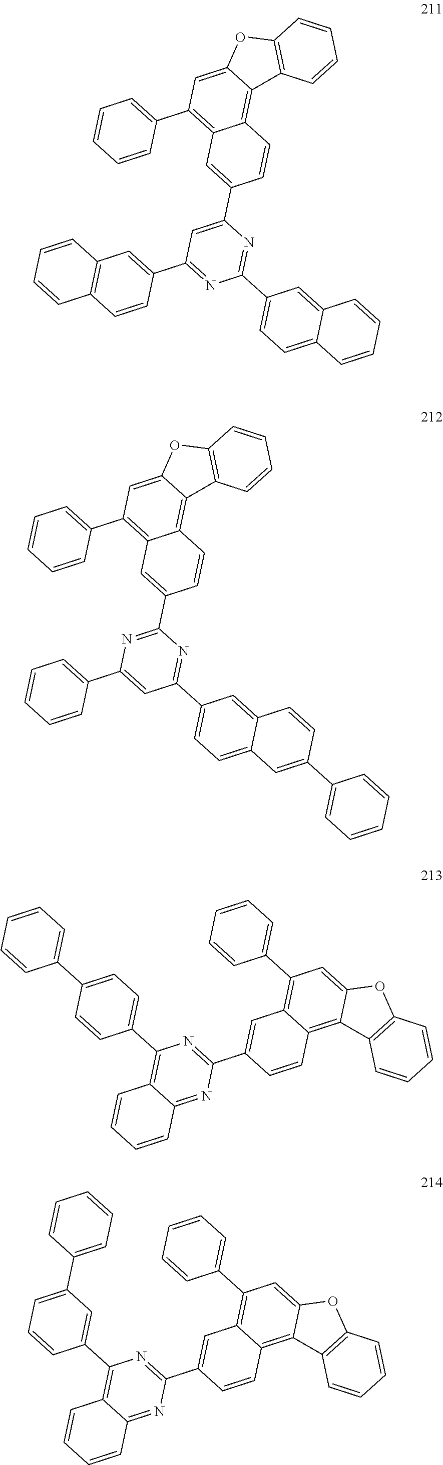 [SMILES: c1ccc(-c2cc3oc4ccccc4c3c3ccc(-c4cc(-c5ccc6ccccc6c5)nc(-c5ccc6ccccc6c5)n4)cc23)cc1 ; c1ccc(-c2ccc3cc(-c4cc(-c5ccccc5)nc(-c5ccc6c(c5)c(-c5ccccc5)cc5oc7ccccc7c56)n4)ccc3c2)cc1 ; c1ccc(-c2ccc(-c3nc(-c4ccc5c(c4)c(-c4ccccc4)cc4oc6ccccc6c45)nc4ccccc34)cc2)cc1 ; c1ccc(-c2cccc(-c3nc(-c4ccc5c(c4)c(-c4ccccc4)cc4oc6ccccc6c45)nc4ccccc34)c2)cc1]

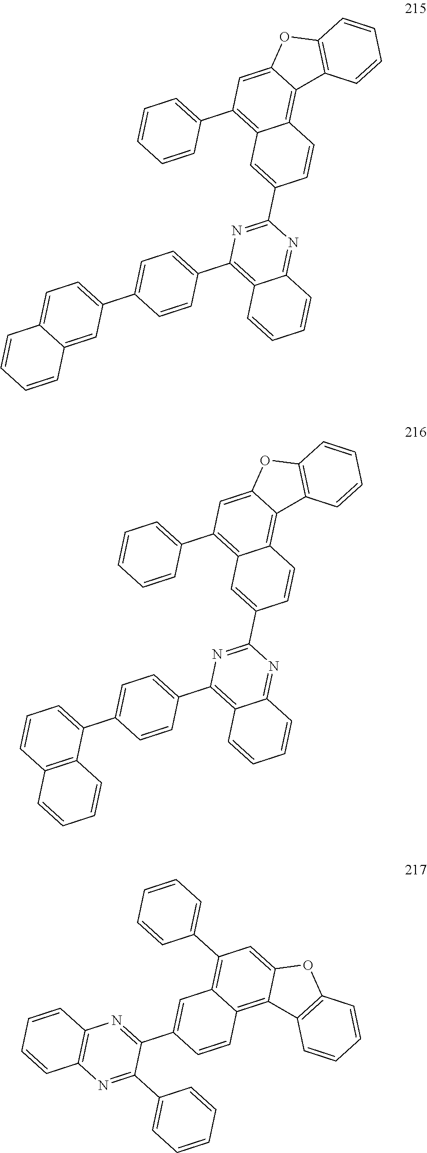 [SMILES: c1ccc(-c2cc3oc4ccccc4c3c3ccc(-c4nc(-c5ccc(-c6ccc7ccccc7c6)cc5)c5ccccc5n4)cc23)cc1 ; c1ccc(-c2cc3oc4ccccc4c3c3ccc(-c4nc(-c5ccc(-c6cccc7ccccc67)cc5)c5ccccc5n4)cc23)cc1 ; c1ccc(-c2nc3ccccc3nc2-c2ccc3c(c2)c(-c2ccccc2)cc2oc4ccccc4c23)cc1]

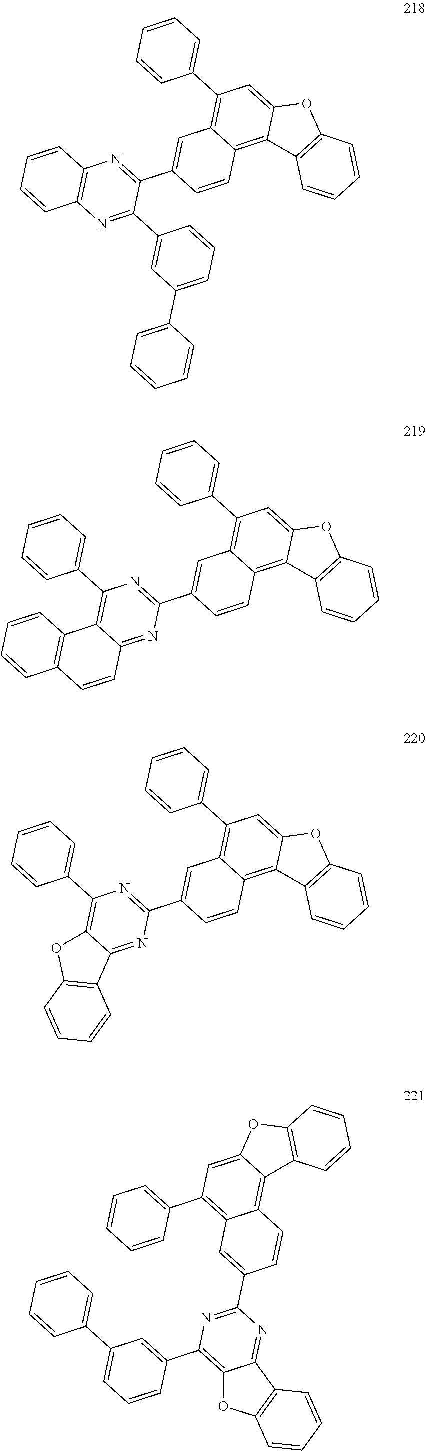 [SMILES: c1ccc(-c2cccc(-c3nc4ccccc4nc3-c3ccc4c(c3)c(-c3ccccc3)cc3oc5ccccc5c34)c2)cc1 ; c1ccc(-c2cc3oc4ccccc4c3c3ccc(-c4nc(-c5ccccc5)c5c(ccc6ccccc65)n4)cc23)cc1 ; c1ccc(-c2cc3oc4ccccc4c3c3ccc(-c4nc(-c5ccccc5)c5oc6ccccc6c5n4)cc23)cc1 ; c1ccc(-c2cccc(-c3nc(-c4ccc5c(c4)c(-c4ccccc4)cc4oc6ccccc6c45)nc4c3oc3ccccc34)c2)cc1]

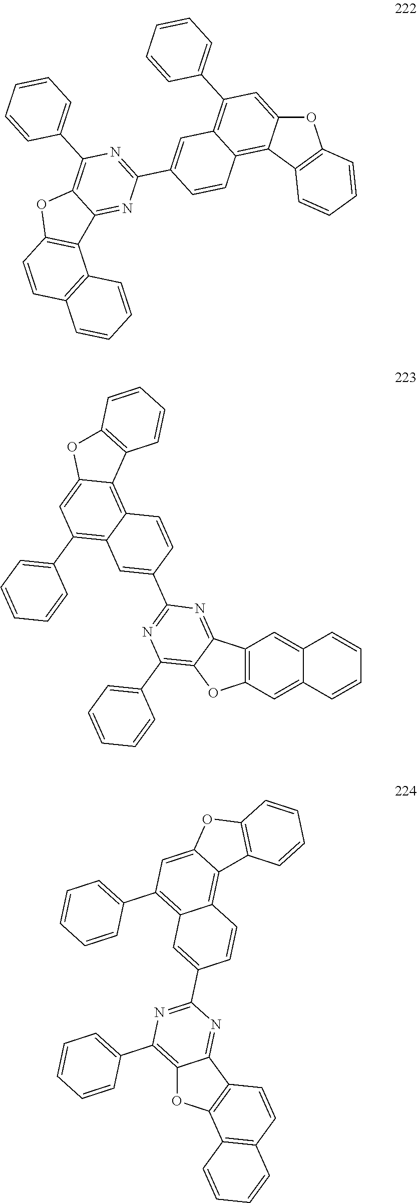 [SMILES: c1ccc(-c2cc3oc4ccccc4c3c3ccc(-c4nc(-c5ccccc5)c5oc6ccc7ccccc7c6c5n4)cc23)cc1 ; c1ccc(-c2cc3oc4ccccc4c3c3ccc(-c4nc(-c5ccccc5)c5oc6cc7ccccc7cc6c5n4)cc23)cc1 ; c1ccc(-c2cc3oc4ccccc4c3c3ccc(-c4nc(-c5ccccc5)c5oc6c7ccccc7ccc6c5n4)cc23)cc1]

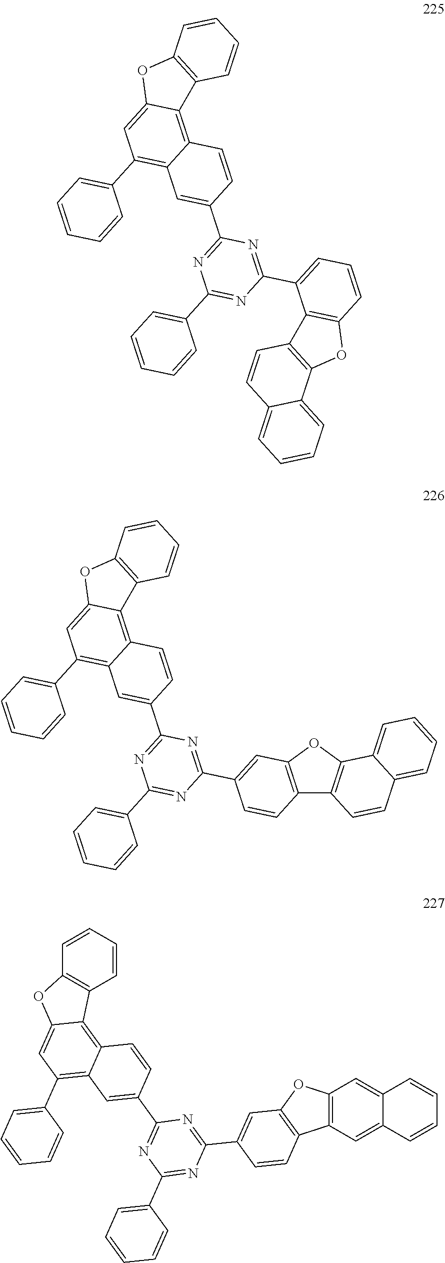 [SMILES: c1ccc(-c2nc(-c3ccc4c(c3)c(-c3ccccc3)cc3oc5ccccc5c34)nc(-c3cccc4oc5c6ccccc6ccc5c34)n2)cc1 ; c1ccc(-c2nc(-c3ccc4c(c3)oc3c5ccccc5ccc43)nc(-c3ccc4c(c3)c(-c3ccccc3)cc3oc5ccccc5c34)n2)cc1 ; c1ccc(-c2nc(-c3ccc4c(c3)oc3cc5ccccc5cc34)nc(-c3ccc4c(c3)c(-c3ccccc3)cc3oc5ccccc5c34)n2)cc1]

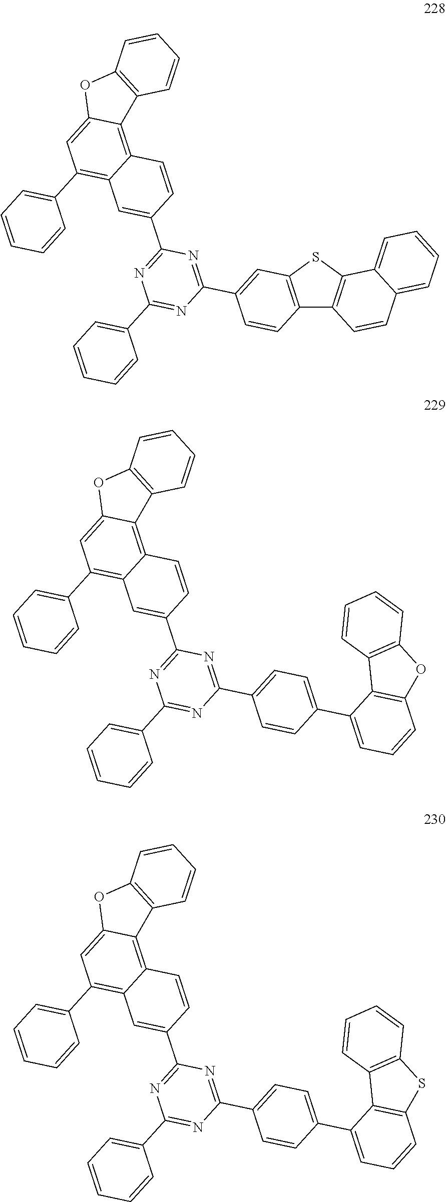 [SMILES: c1ccc(-c2nc(-c3ccc4c(c3)sc3c5ccccc5ccc43)nc(-c3ccc4c(c3)c(-c3ccccc3)cc3oc5ccccc5c34)n2)cc1 ; c1ccc(-c2nc(-c3ccc(-c4cccc5oc6ccccc6c45)cc3)nc(-c3ccc4c(c3)c(-c3ccccc3)cc3oc5ccccc5c34)n2)cc1 ; c1ccc(-c2nc(-c3ccc(-c4cccc5sc6ccccc6c45)cc3)nc(-c3ccc4c(c3)c(-c3ccccc3)cc3oc5ccccc5c34)n2)cc1]

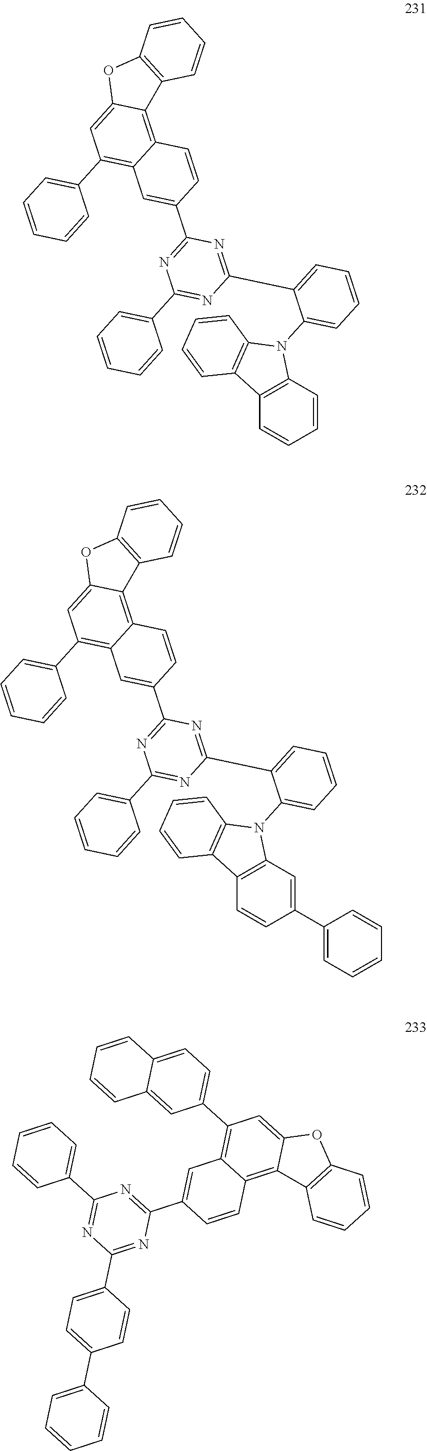 [SMILES: c1ccc(-c2nc(-c3ccc4c(c3)c(-c3ccccc3)cc3oc5ccccc5c34)nc(-c3ccccc3-n3c4ccccc4c4ccccc43)n2)cc1 ; c1ccc(-c2ccc3c4ccccc4n(-c4ccccc4-c4nc(-c5ccccc5)nc(-c5ccc6c(c5)c(-c5ccccc5)cc5oc7ccccc7c56)n4)c3c2)cc1 ; c1ccc(-c2ccc(-c3nc(-c4ccccc4)nc(-c4ccc5c(c4)c(-c4ccc6ccccc6c4)cc4oc6ccccc6c45)n3)cc2)cc1]

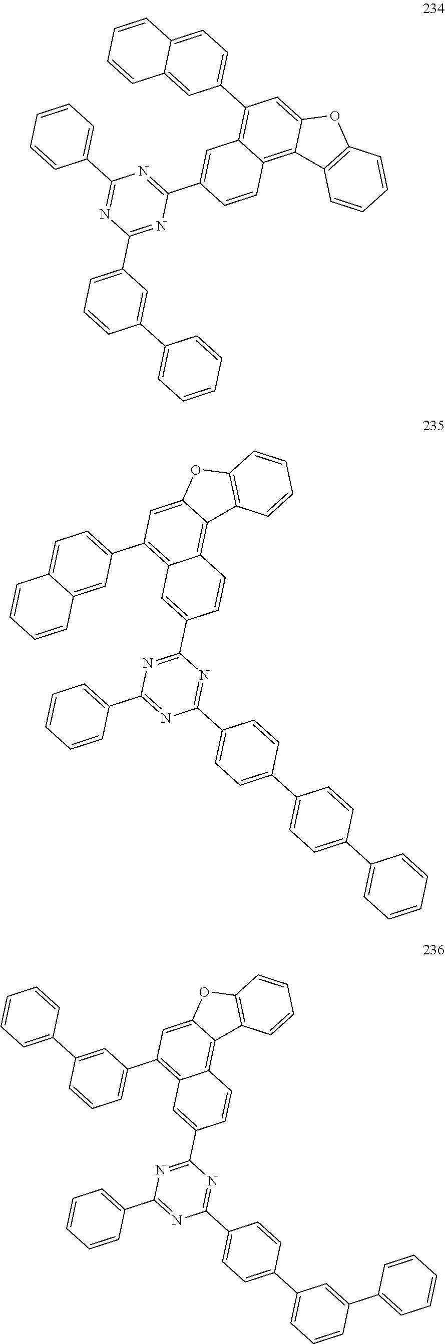 [SMILES: c1ccc(-c2cccc(-c3nc(-c4ccccc4)nc(-c4ccc5c(c4)c(-c4ccc6ccccc6c4)cc4oc6ccccc6c45)n3)c2)cc1 ; c1ccc(-c2ccc(-c3ccc(-c4nc(-c5ccccc5)nc(-c5ccc6c(c5)c(-c5ccc7ccccc7c5)cc5oc7ccccc7c56)n4)cc3)cc2)cc1 ; c1ccc(-c2cccc(-c3ccc(-c4nc(-c5ccccc5)nc(-c5ccc6c(c5)c(-c5cccc(-c7ccccc7)c5)cc5oc7ccccc7c56)n4)cc3)c2)cc1]

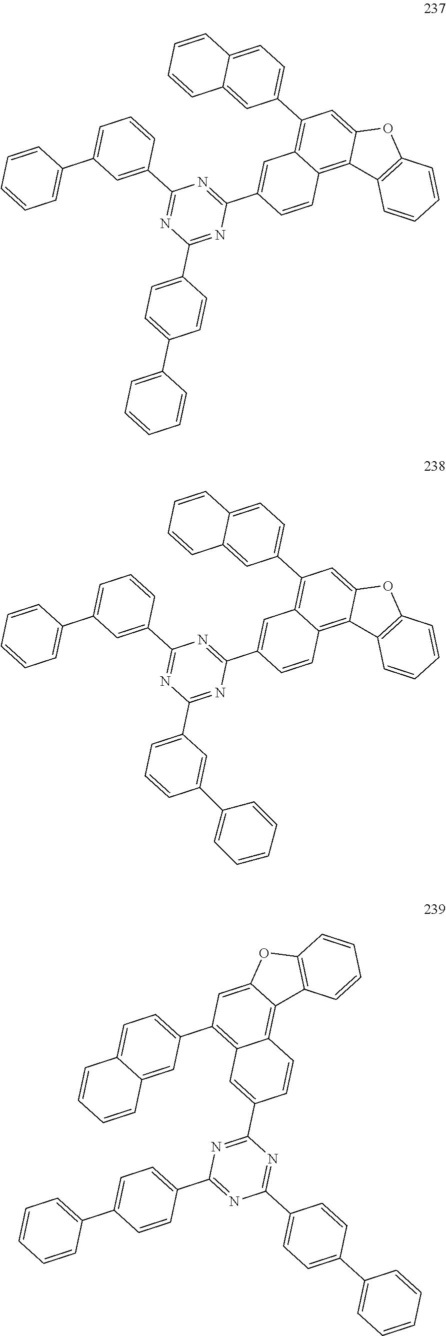 [SMILES: c1ccc(-c2ccc(-c3nc(-c4cccc(-c5ccccc5)c4)nc(-c4ccc5c(c4)c(-c4ccc6ccccc6c4)cc4oc6ccccc6c45)n3)cc2)cc1 ; c1ccc(-c2cccc(-c3nc(-c4cccc(-c5ccccc5)c4)nc(-c4ccc5c(c4)c(-c4ccc6ccccc6c4)cc4oc6ccccc6c45)n3)c2)cc1 ; c1ccc(-c2ccc(-c3nc(-c4ccc(-c5ccccc5)cc4)nc(-c4ccc5c(c4)c(-c4ccc6ccccc6c4)cc4oc6ccccc6c45)n3)cc2)cc1]

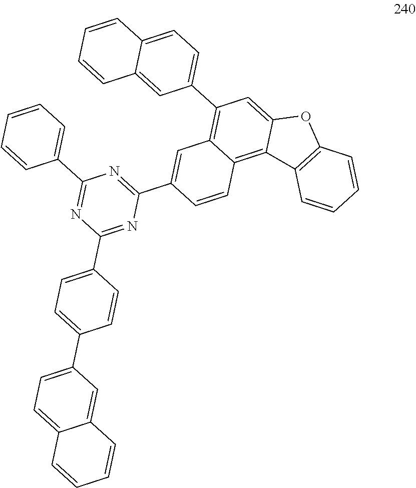 [SMILES: c1ccc(-c2nc(-c3ccc(-c4ccc5ccccc5c4)cc3)nc(-c3ccc4c(c3)c(-c3ccc5ccccc5c3)cc3oc5ccccc5c34)n2)cc1]

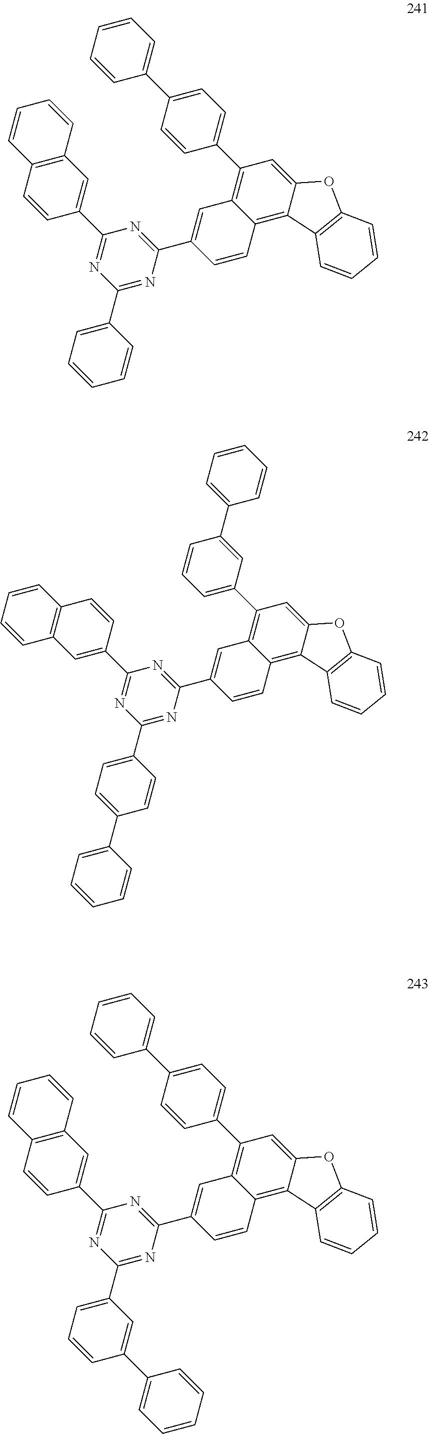 [SMILES: c1ccc(-c2ccc(-c3cc4oc5ccccc5c4c4ccc(-c5nc(-c6ccccc6)nc(-c6ccc7ccccc7c6)n5)cc34)cc2)cc1 ; c1ccc(-c2ccc(-c3nc(-c4ccc5ccccc5c4)nc(-c4ccc5c(c4)c(-c4cccc(-c6ccccc6)c4)cc4oc6ccccc6c45)n3)cc2)cc1 ; c1ccc(-c2ccc(-c3cc4oc5ccccc5c4c4ccc(-c5nc(-c6cccc(-c7ccccc7)c6)nc(-c6ccc7ccccc7c6)n5)cc34)cc2)cc1]

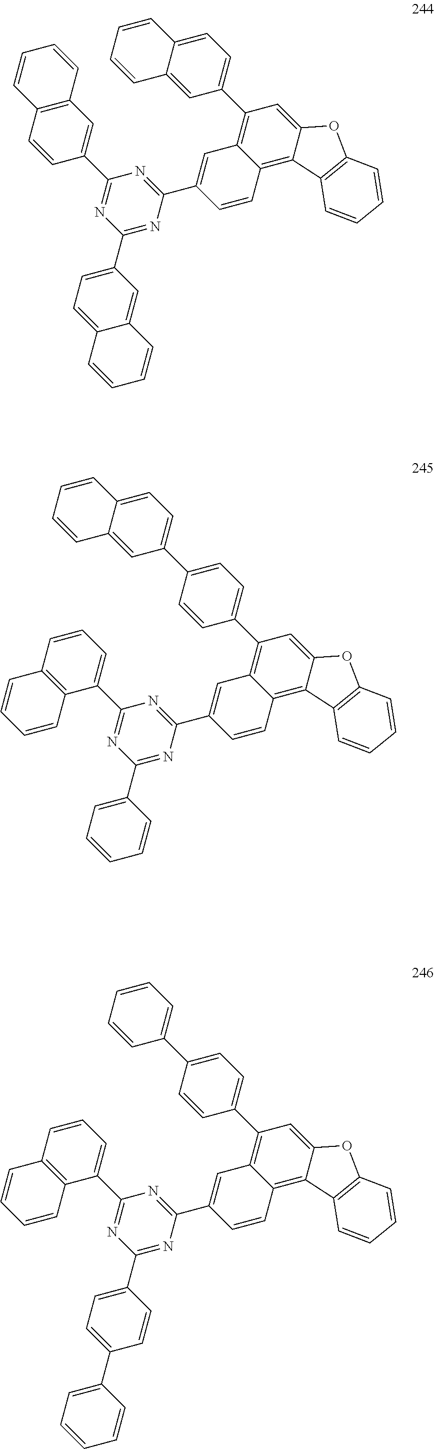 [SMILES: c1ccc2cc(-c3nc(-c4ccc5ccccc5c4)nc(-c4ccc5c(c4)c(-c4ccc6ccccc6c4)cc4oc6ccccc6c45)n3)ccc2c1 ; c1ccc(-c2nc(-c3ccc4c(c3)c(-c3ccc(-c5ccc6ccccc6c5)cc3)cc3oc5ccccc5c34)nc(-c3cccc4ccccc34)n2)cc1 ; c1ccc(-c2ccc(-c3nc(-c4ccc5c(c4)c(-c4ccc(-c6ccccc6)cc4)cc4oc6ccccc6c45)nc(-c4cccc5ccccc45)n3)cc2)cc1]

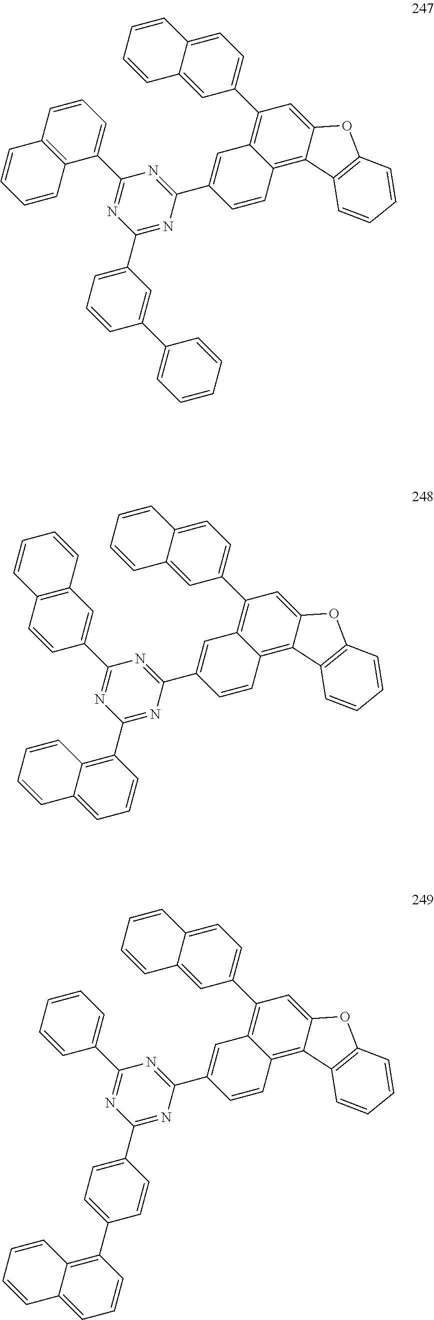 [SMILES: c1ccc(-c2cccc(-c3nc(-c4ccc5c(c4)c(-c4ccc6ccccc6c4)cc4oc6ccccc6c45)nc(-c4cccc5ccccc45)n3)c2)cc1 ; c1ccc2cc(-c3nc(-c4ccc5c(c4)c(-c4ccc6ccccc6c4)cc4oc6ccccc6c45)nc(-c4cccc5ccccc45)n3)ccc2c1 ; c1ccc(-c2nc(-c3ccc(-c4cccc5ccccc45)cc3)nc(-c3ccc4c(c3)c(-c3ccc5ccccc5c3)cc3oc5ccccc5c34)n2)cc1]

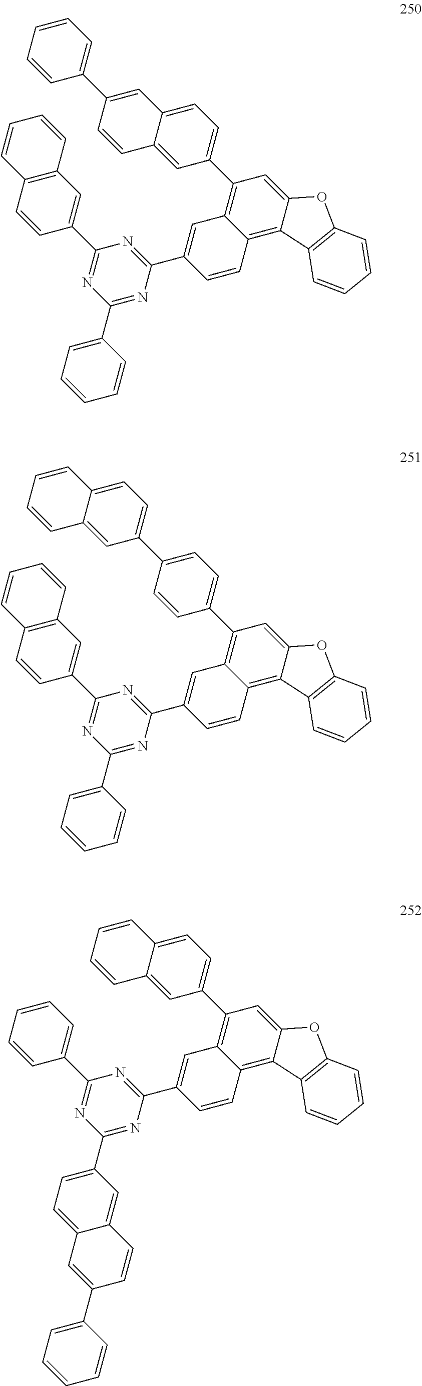 [SMILES: c1ccc(-c2ccc3cc(-c4cc5oc6ccccc6c5c5ccc(-c6nc(-c7ccccc7)nc(-c7ccc8ccccc8c7)n6)cc45)ccc3c2)cc1 ; c1ccc(-c2nc(-c3ccc4ccccc4c3)nc(-c3ccc4c(c3)c(-c3ccc(-c5ccc6ccccc6c5)cc3)cc3oc5ccccc5c34)n2)cc1 ; c1ccc(-c2ccc3cc(-c4nc(-c5ccccc5)nc(-c5ccc6c(c5)c(-c5ccc7ccccc7c5)cc5oc7ccccc7c56)n4)ccc3c2)cc1]

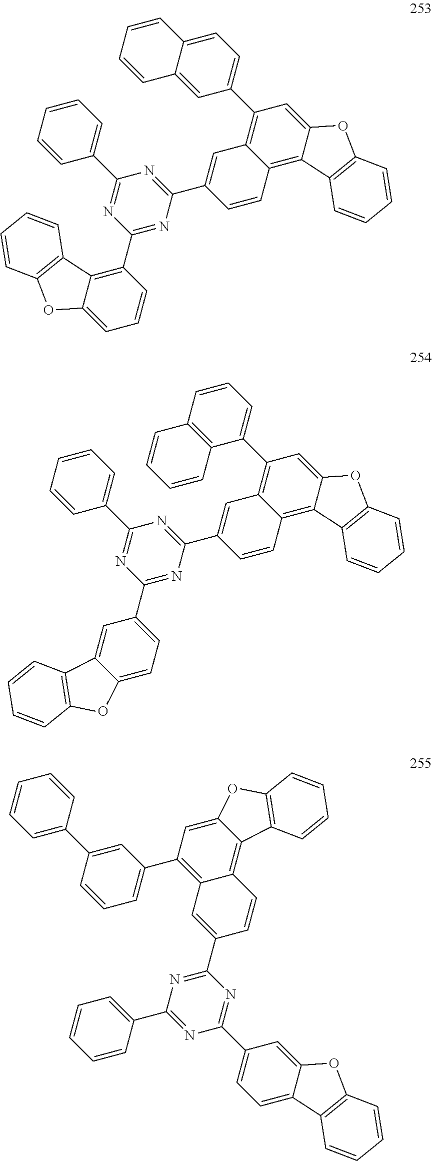 [SMILES: c1ccc(-c2nc(-c3ccc4c(c3)c(-c3ccc5ccccc5c3)cc3oc5ccccc5c34)nc(-c3cccc4oc5ccccc5c34)n2)cc1 ; c1ccc(-c2nc(-c3ccc4c(c3)c(-c3cccc5ccccc35)cc3oc5ccccc5c34)nc(-c3ccc4oc5ccccc5c4c3)n2)cc1 ; c1ccc(-c2cccc(-c3cc4oc5ccccc5c4c4ccc(-c5nc(-c6ccccc6)nc(-c6ccc7c(c6)oc6ccccc67)n5)cc34)c2)cc1]

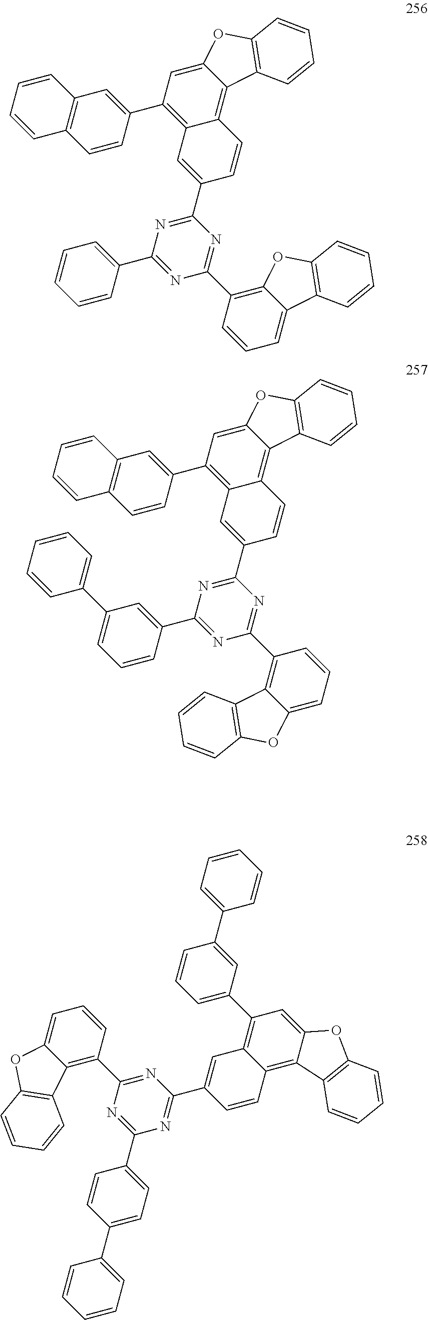 [SMILES: c1ccc(-c2nc(-c3ccc4c(c3)c(-c3ccc5ccccc5c3)cc3oc5ccccc5c34)nc(-c3cccc4c3oc3ccccc34)n2)cc1 ; c1ccc(-c2cccc(-c3nc(-c4ccc5c(c4)c(-c4ccc6ccccc6c4)cc4oc6ccccc6c45)nc(-c4cccc5oc6ccccc6c45)n3)c2)cc1 ; c1ccc(-c2ccc(-c3nc(-c4ccc5c(c4)c(-c4cccc(-c6ccccc6)c4)cc4oc6ccccc6c45)nc(-c4cccc5oc6ccccc6c45)n3)cc2)cc1]

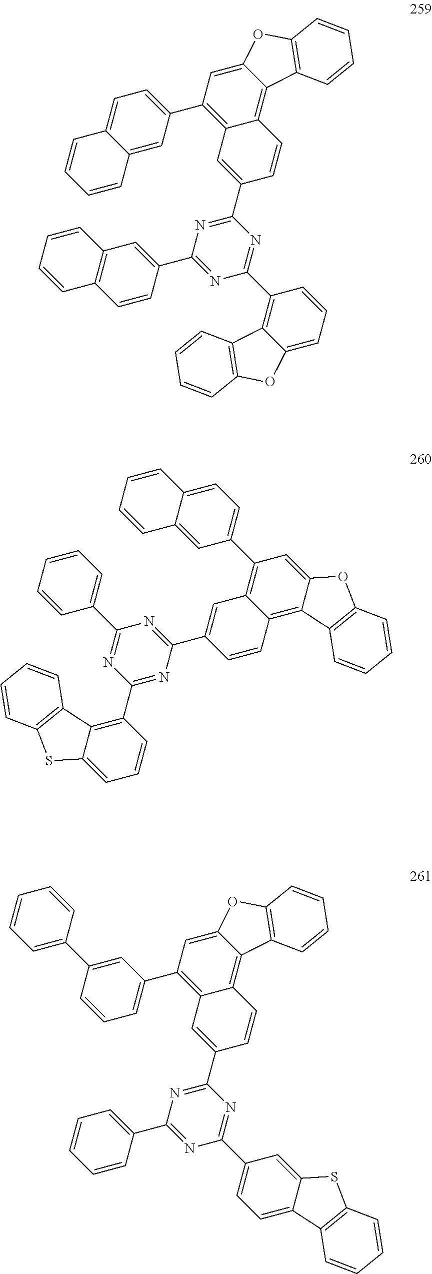 [SMILES: c1ccc2cc(-c3nc(-c4ccc5c(c4)c(-c4ccc6ccccc6c4)cc4oc6ccccc6c45)nc(-c4cccc5oc6ccccc6c45)n3)ccc2c1 ; c1ccc(-c2nc(-c3ccc4c(c3)c(-c3ccc5ccccc5c3)cc3oc5ccccc5c34)nc(-c3cccc4sc5ccccc5c34)n2)cc1 ; c1ccc(-c2cccc(-c3cc4oc5ccccc5c4c4ccc(-c5nc(-c6ccccc6)nc(-c6ccc7c(c6)sc6ccccc67)n5)cc34)c2)cc1]

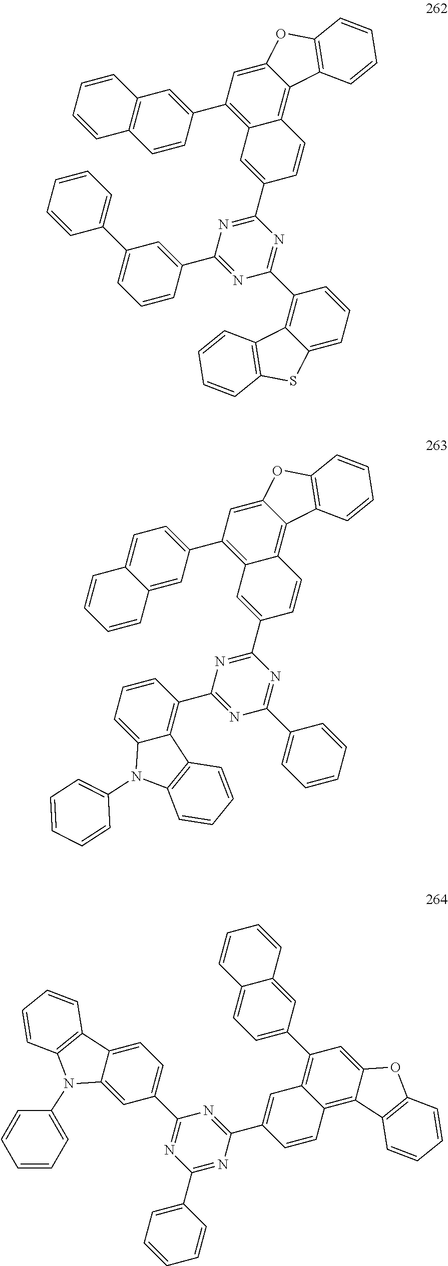 [SMILES: c1ccc(-c2cccc(-c3nc(-c4ccc5c(c4)c(-c4ccc6ccccc6c4)cc4oc6ccccc6c45)nc(-c4cccc5sc6ccccc6c45)n3)c2)cc1 ; c1ccc(-c2nc(-c3ccc4c(c3)c(-c3ccc5ccccc5c3)cc3oc5ccccc5c34)nc(-c3cccc4c3c3ccccc3n4-c3ccccc3)n2)cc1 ; c1ccc(-c2nc(-c3ccc4c(c3)c(-c3ccc5ccccc5c3)cc3oc5ccccc5c34)nc(-c3ccc4c5ccccc5n(-c5ccccc5)c4c3)n2)cc1]

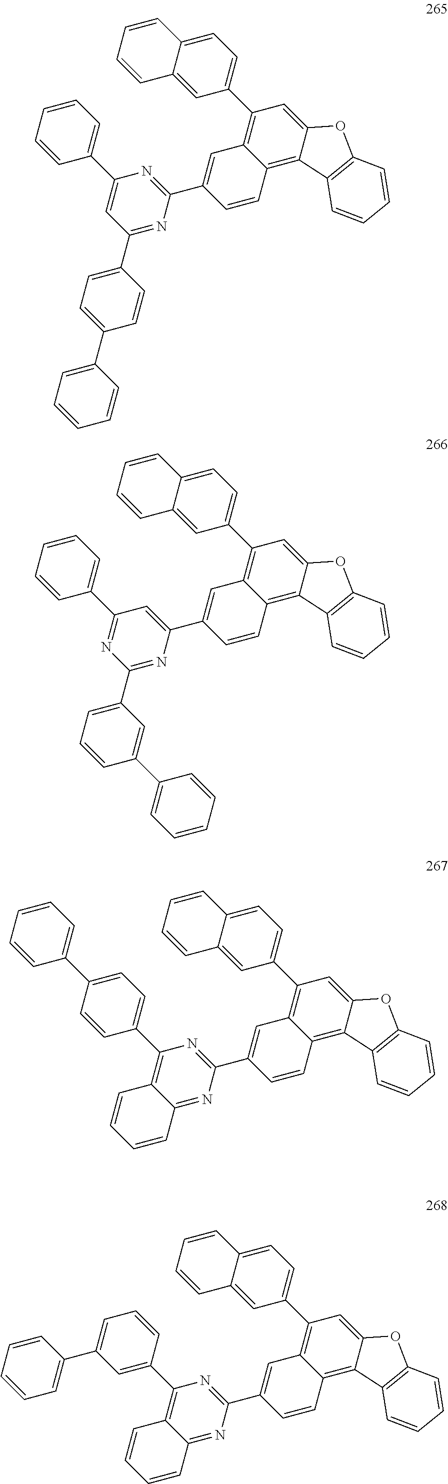

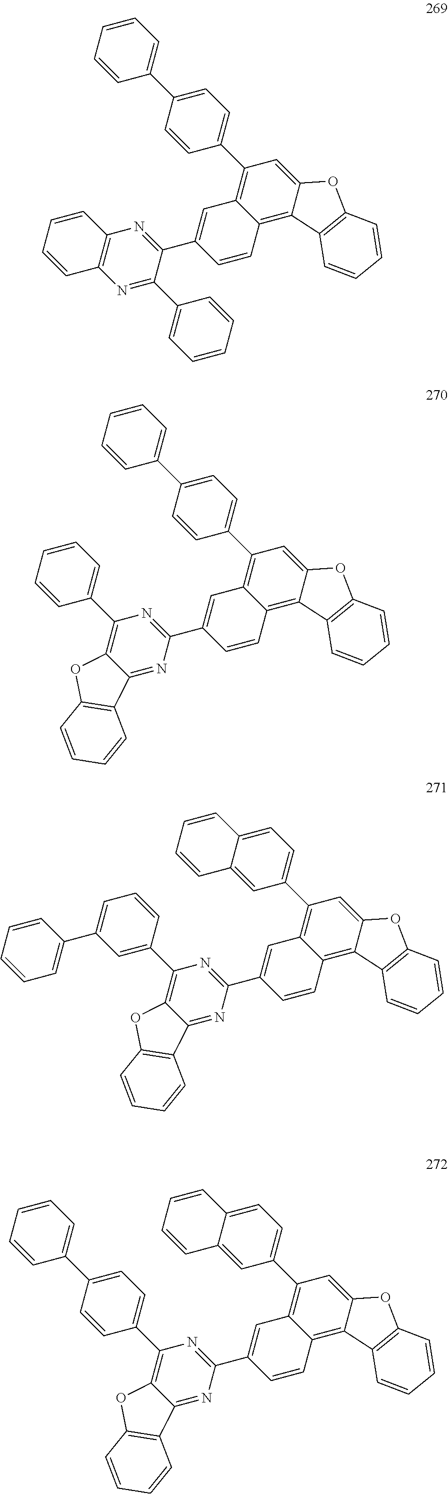 [SMILES: c1ccc(-c2ccc(-c3cc4oc5ccccc5c4c4ccc(-c5nc6ccccc6nc5-c5ccccc5)cc34)cc2)cc1 ; c1ccc(-c2ccc(-c3cc4oc5ccccc5c4c4ccc(-c5nc(-c6ccccc6)c6oc7ccccc7c6n5)cc34)cc2)cc1 ; c1ccc(-c2cccc(-c3nc(-c4ccc5c(c4)c(-c4ccc6ccccc6c4)cc4oc6ccccc6c45)nc4c3oc3ccccc34)c2)cc1 ; c1ccc(-c2ccc(-c3nc(-c4ccc5c(c4)c(-c4ccc6ccccc6c4)cc4oc6ccccc6c45)nc4c3oc3ccccc34)cc2)cc1]

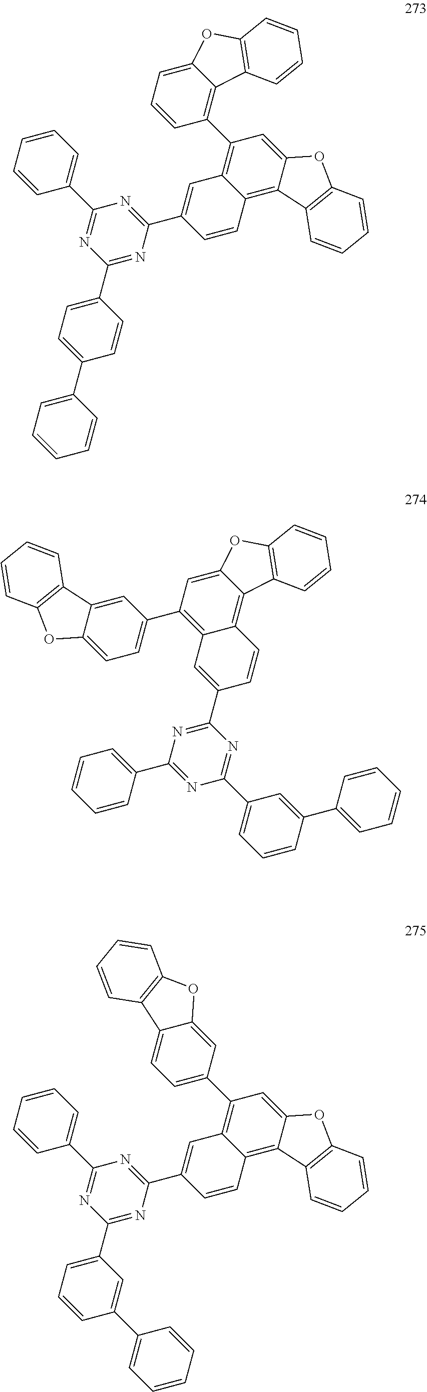 [SMILES: c1ccc(-c2ccc(-c3nc(-c4ccccc4)nc(-c4ccc5c(c4)c(-c4cccc6oc7ccccc7c46)cc4oc6ccccc6c45)n3)cc2)cc1 ; c1ccc(-c2cccc(-c3nc(-c4ccccc4)nc(-c4ccc5c(c4)c(-c4ccc6oc7ccccc7c6c4)cc4oc6ccccc6c45)n3)c2)cc1 ; c1ccc(-c2cccc(-c3nc(-c4ccccc4)nc(-c4ccc5c(c4)c(-c4ccc6c(c4)oc4ccccc46)cc4oc6ccccc6c45)n3)c2)cc1]

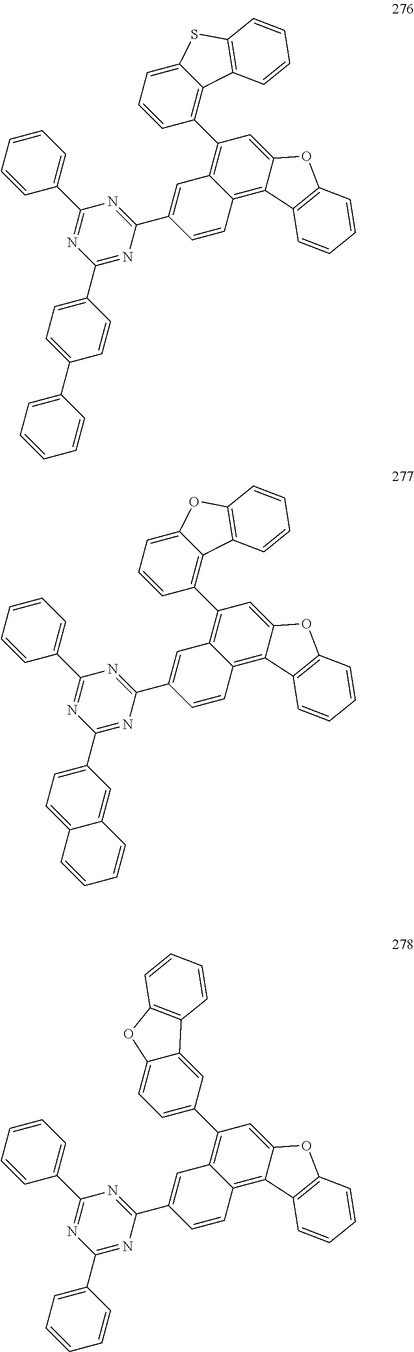 [SMILES: c1ccc(-c2ccc(-c3nc(-c4ccccc4)nc(-c4ccc5c(c4)c(-c4cccc6sc7ccccc7c46)cc4oc6ccccc6c45)n3)cc2)cc1 ; c1ccc(-c2nc(-c3ccc4ccccc4c3)nc(-c3ccc4c(c3)c(-c3cccc5oc6ccccc6c35)cc3oc5ccccc5c34)n2)cc1 ; c1ccc(-c2nc(-c3ccccc3)nc(-c3ccc4c(c3)c(-c3ccc5oc6ccccc6c5c3)cc3oc5ccccc5c34)n2)cc1]

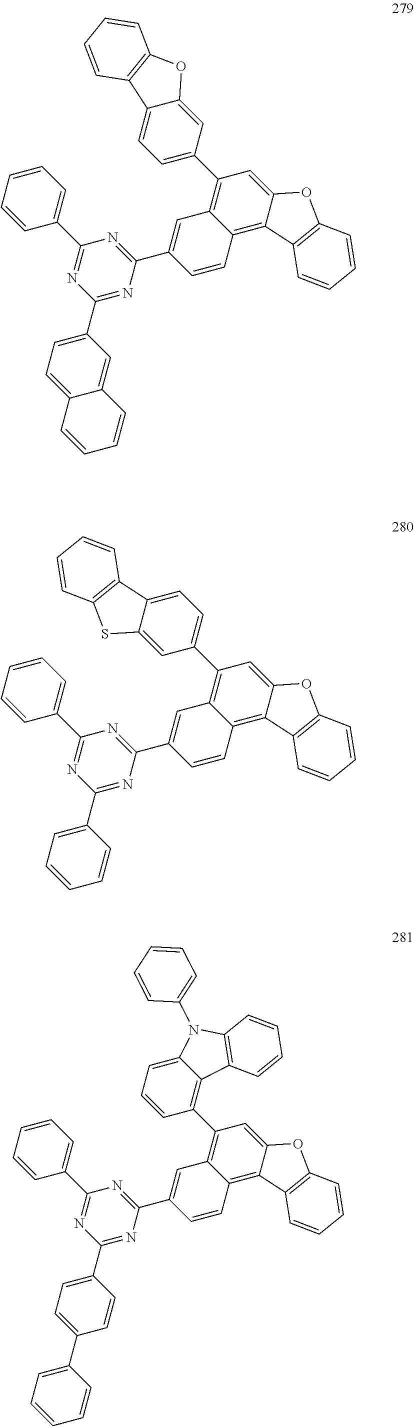 [SMILES: c1ccc(-c2nc(-c3ccc4ccccc4c3)nc(-c3ccc4c(c3)c(-c3ccc5c(c3)oc3ccccc35)cc3oc5ccccc5c34)n2)cc1 ; c1ccc(-c2nc(-c3ccccc3)nc(-c3ccc4c(c3)c(-c3ccc5c(c3)sc3ccccc35)cc3oc5ccccc5c34)n2)cc1 ; c1ccc(-c2ccc(-c3nc(-c4ccccc4)nc(-c4ccc5c(c4)c(-c4cccc6c4c4ccccc4n6-c4ccccc4)cc4oc6ccccc6c45)n3)cc2)cc1]

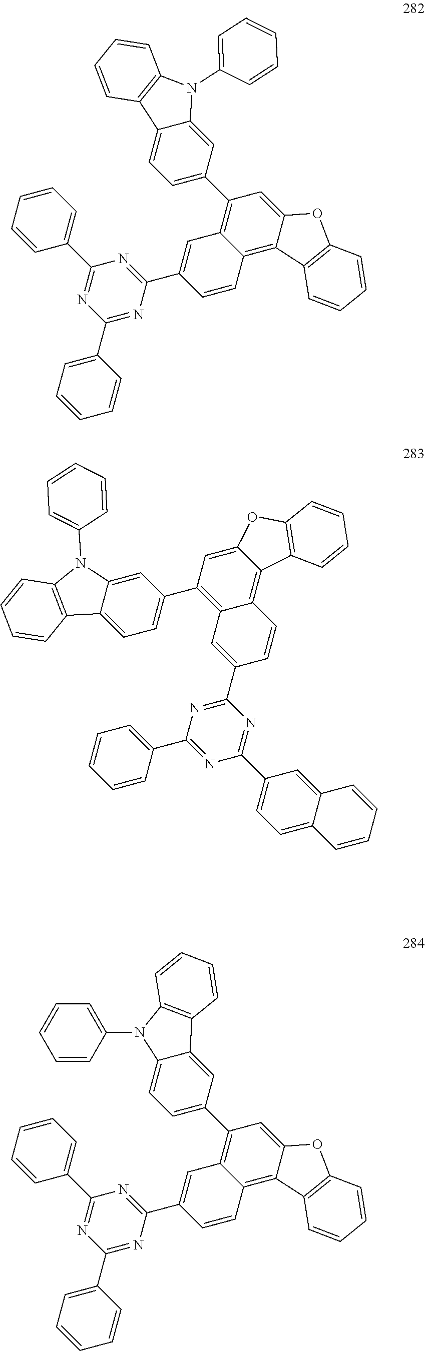

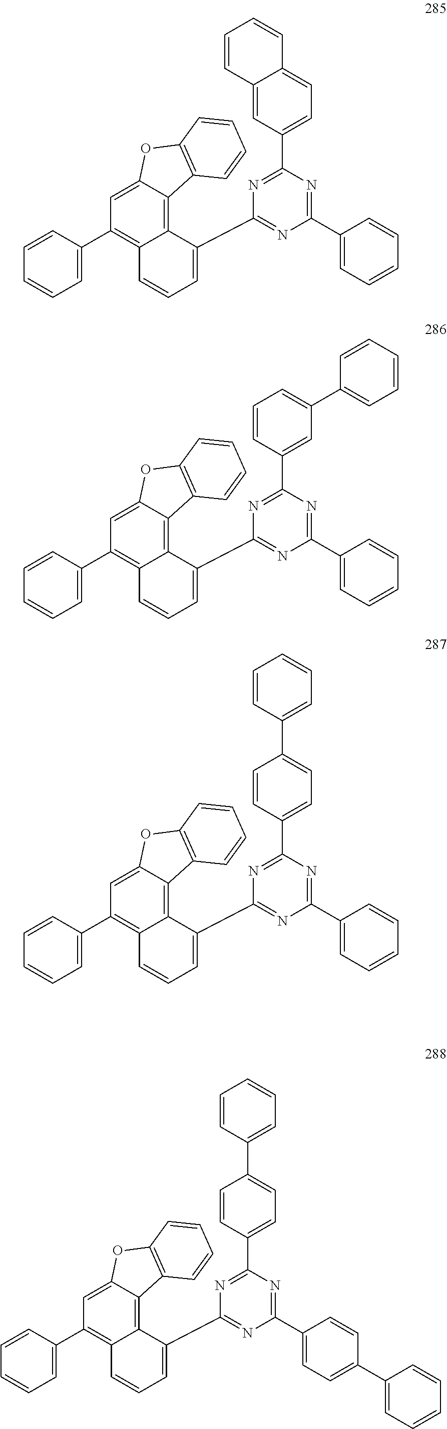 [SMILES: c1ccc(-c2nc(-c3ccc4ccccc4c3)nc(-c3cccc4c(-c5ccccc5)cc5oc6ccccc6c5c34)n2)cc1 ; c1ccc(-c2cccc(-c3nc(-c4ccccc4)nc(-c4cccc5c(-c6ccccc6)cc6oc7ccccc7c6c45)n3)c2)cc1 ; c1ccc(-c2ccc(-c3nc(-c4ccccc4)nc(-c4cccc5c(-c6ccccc6)cc6oc7ccccc7c6c45)n3)cc2)cc1 ; c1ccc(-c2ccc(-c3nc(-c4ccc(-c5ccccc5)cc4)nc(-c4cccc5c(-c6ccccc6)cc6oc7ccccc7c6c45)n3)cc2)cc1]

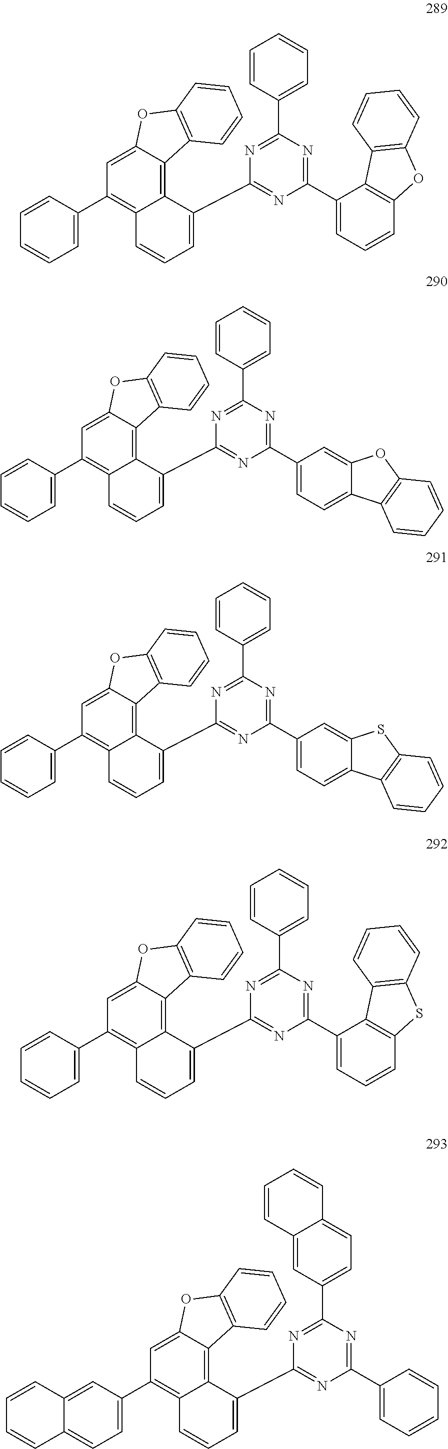 [SMILES: c1ccc(-c2nc(-c3cccc4oc5ccccc5c34)nc(-c3cccc4c(-c5ccccc5)cc5oc6ccccc6c5c34)n2)cc1 ; c1ccc(-c2nc(-c3ccc4c(c3)oc3ccccc34)nc(-c3cccc4c(-c5ccccc5)cc5oc6ccccc6c5c34)n2)cc1 ; c1ccc(-c2nc(-c3ccc4c(c3)sc3ccccc34)nc(-c3cccc4c(-c5ccccc5)cc5oc6ccccc6c5c34)n2)cc1 ; c1ccc(-c2nc(-c3cccc4sc5ccccc5c34)nc(-c3cccc4c(-c5ccccc5)cc5oc6ccccc6c5c34)n2)cc1 ; c1ccc(-c2nc(-c3ccc4ccccc4c3)nc(-c3cccc4c(-c5ccc6ccccc6c5)cc5oc6ccccc6c5c34)n2)cc1]

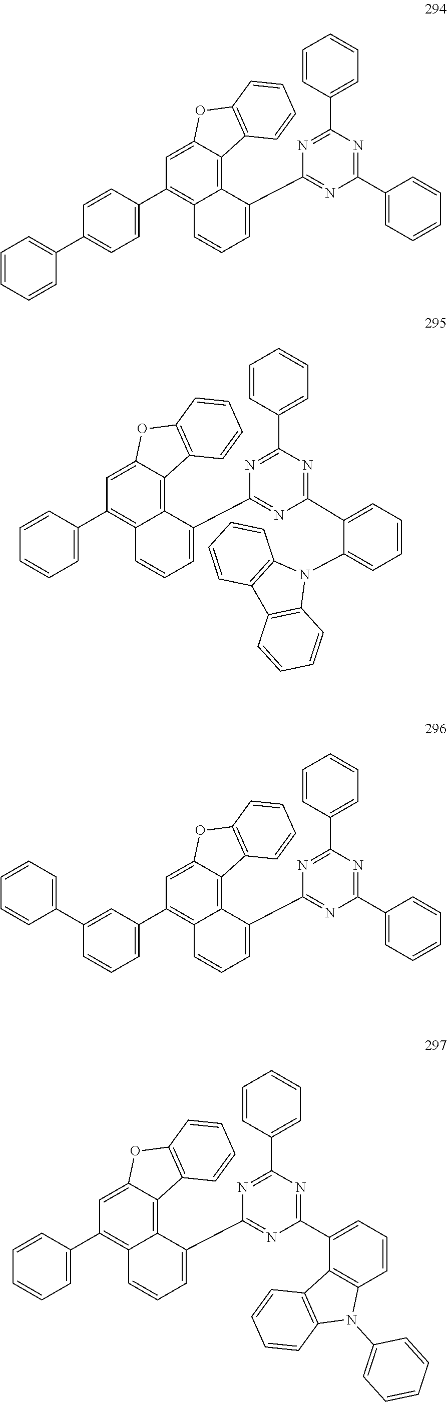 [SMILES: c1ccc(-c2ccc(-c3cc4oc5ccccc5c4c4c(-c5nc(-c6ccccc6)nc(-c6ccccc6)n5)cccc34)cc2)cc1 ; c1ccc(-c2nc(-c3ccccc3-n3c4ccccc4c4ccccc43)nc(-c3cccc4c(-c5ccccc5)cc5oc6ccccc6c5c34)n2)cc1 ; c1ccc(-c2cccc(-c3cc4oc5ccccc5c4c4c(-c5nc(-c6ccccc6)nc(-c6ccccc6)n5)cccc34)c2)cc1 ; c1ccc(-c2nc(-c3cccc4c(-c5ccccc5)cc5oc6ccccc6c5c34)nc(-c3cccc4c3c3ccccc3n4-c3ccccc3)n2)cc1]

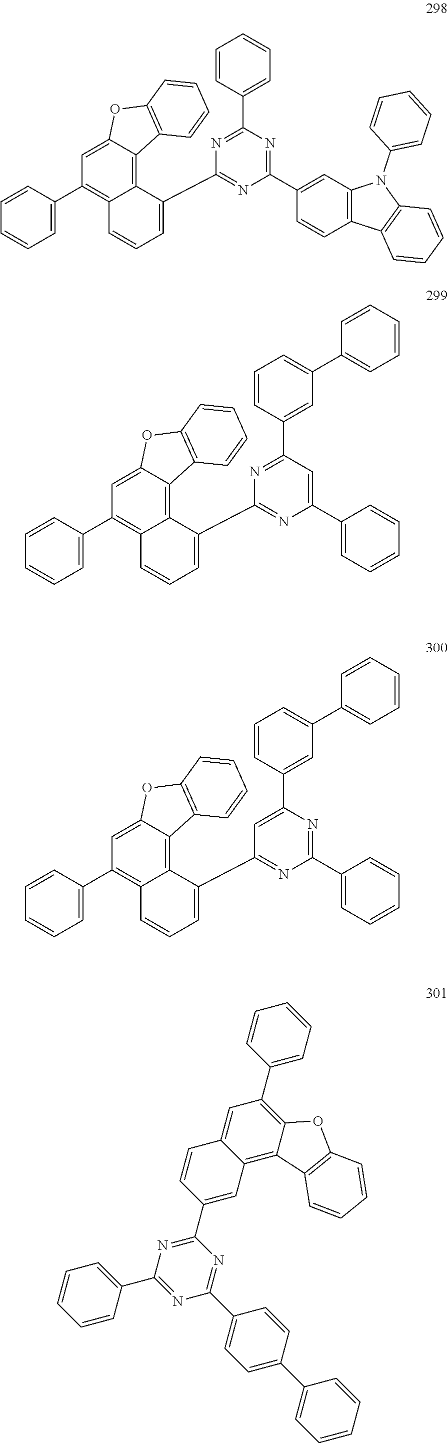 [SMILES: c1ccc(-c2nc(-c3ccc4c5ccccc5n(-c5ccccc5)c4c3)nc(-c3cccc4c(-c5ccccc5)cc5oc6ccccc6c5c34)n2)cc1 ; c1ccc(-c2cccc(-c3cc(-c4ccccc4)nc(-c4cccc5c(-c6ccccc6)cc6oc7ccccc7c6c45)n3)c2)cc1 ; c1ccc(-c2cccc(-c3cc(-c4cccc5c(-c6ccccc6)cc6oc7ccccc7c6c45)nc(-c4ccccc4)n3)c2)cc1 ; c1ccc(-c2ccc(-c3nc(-c4ccccc4)nc(-c4ccc5cc(-c6ccccc6)c6oc7ccccc7c6c5c4)n3)cc2)cc1]

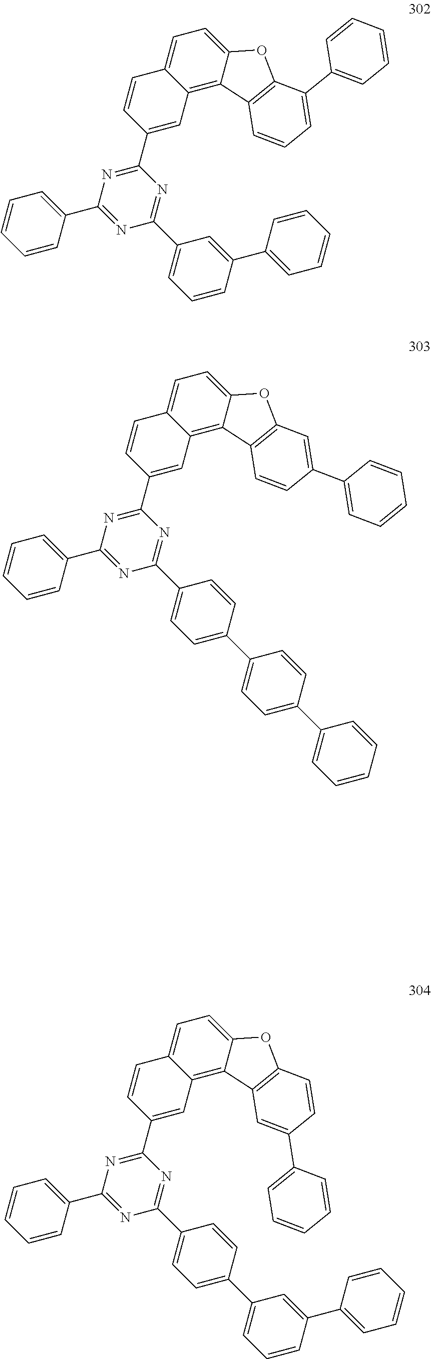 [SMILES: c1ccc(-c2cccc(-c3nc(-c4ccccc4)nc(-c4ccc5ccc6oc7c(-c8ccccc8)cccc7c6c5c4)n3)c2)cc1 ; c1ccc(-c2ccc(-c3ccc(-c4nc(-c5ccccc5)nc(-c5ccc6ccc7oc8cc(-c9ccccc9)ccc8c7c6c5)n4)cc3)cc2)cc1 ; c1ccc(-c2cccc(-c3ccc(-c4nc(-c5ccccc5)nc(-c5ccc6ccc7oc8ccc(-c9ccccc9)cc8c7c6c5)n4)cc3)c2)cc1]

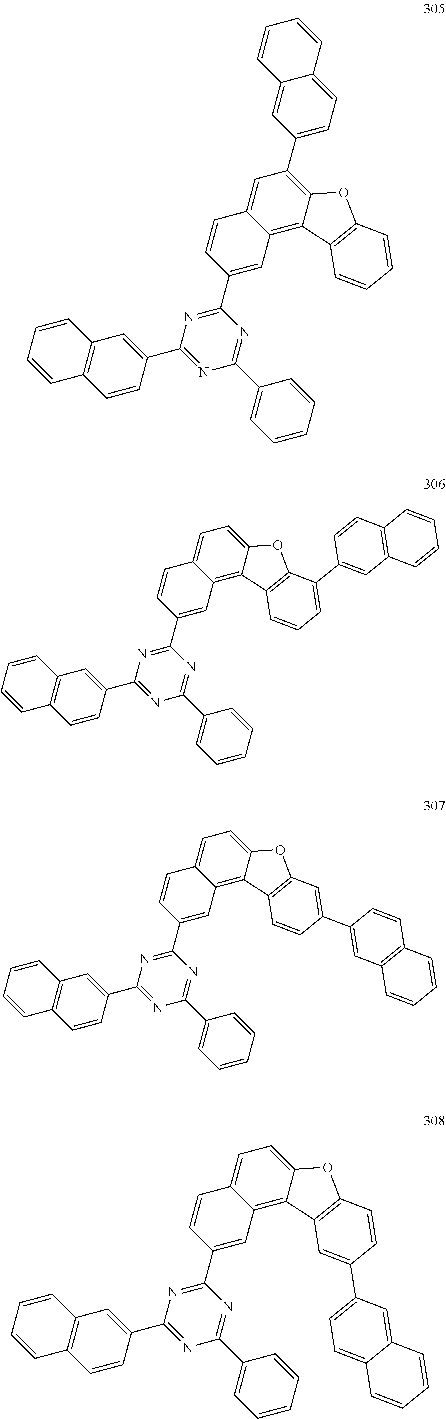 [SMILES: c1ccc(-c2nc(-c3ccc4ccccc4c3)nc(-c3ccc4cc(-c5ccc6ccccc6c5)c5oc6ccccc6c5c4c3)n2)cc1 ; c1ccc(-c2nc(-c3ccc4ccccc4c3)nc(-c3ccc4ccc5oc6c(-c7ccc8ccccc8c7)cccc6c5c4c3)n2)cc1 ; c1ccc(-c2nc(-c3ccc4ccccc4c3)nc(-c3ccc4ccc5oc6cc(-c7ccc8ccccc8c7)ccc6c5c4c3)n2)cc1 ; c1ccc(-c2nc(-c3ccc4ccccc4c3)nc(-c3ccc4ccc5oc6ccc(-c7ccc8ccccc8c7)cc6c5c4c3)n2)cc1]

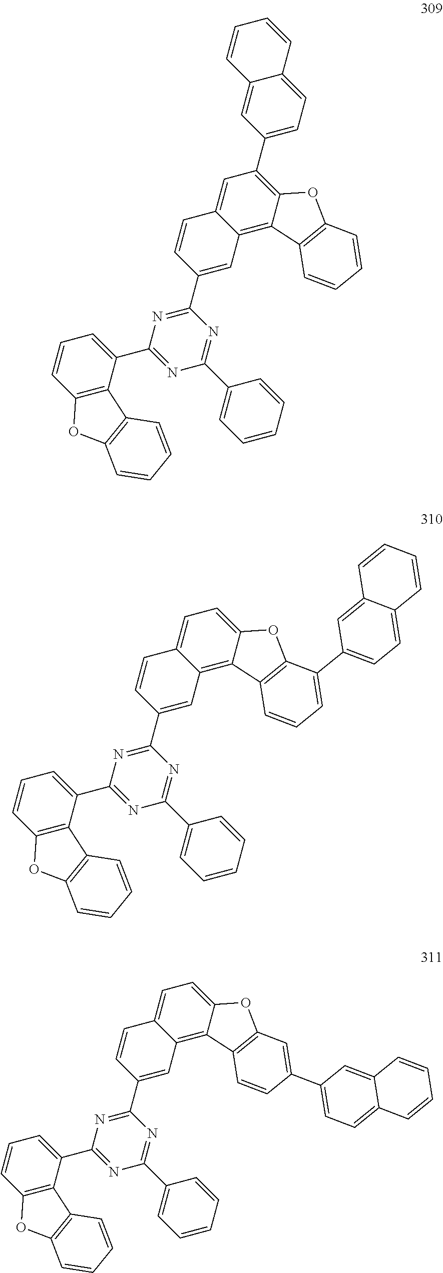 [SMILES: c1ccc(-c2nc(-c3ccc4cc(-c5ccc6ccccc6c5)c5oc6ccccc6c5c4c3)nc(-c3cccc4oc5ccccc5c34)n2)cc1 ; c1ccc(-c2nc(-c3ccc4ccc5oc6c(-c7ccc8ccccc8c7)cccc6c5c4c3)nc(-c3cccc4oc5ccccc5c34)n2)cc1 ; c1ccc(-c2nc(-c3ccc4ccc5oc6cc(-c7ccc8ccccc8c7)ccc6c5c4c3)nc(-c3cccc4oc5ccccc5c34)n2)cc1]

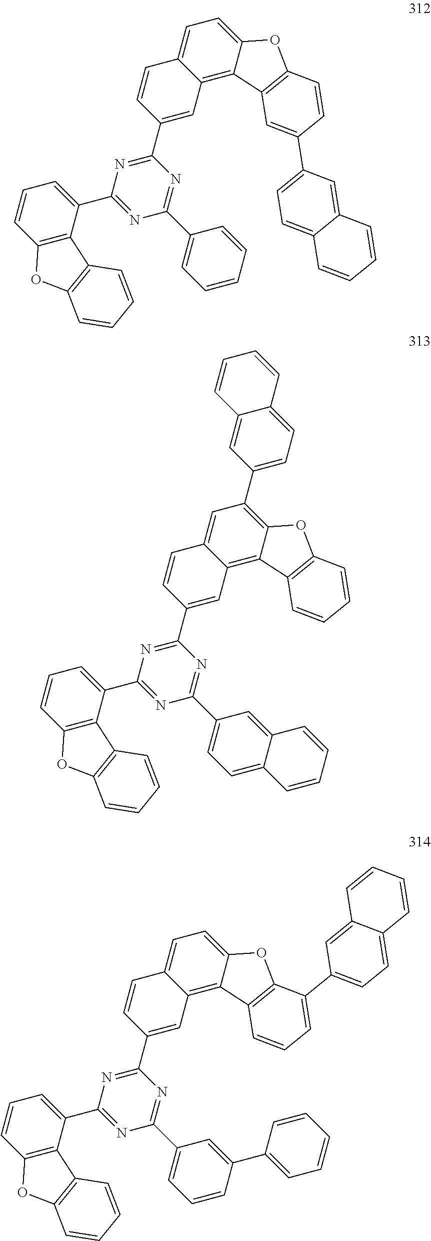 [SMILES: c1ccc(-c2nc(-c3ccc4ccc5oc6ccc(-c7ccc8ccccc8c7)cc6c5c4c3)nc(-c3cccc4oc5ccccc5c34)n2)cc1 ; c1ccc2cc(-c3nc(-c4ccc5cc(-c6ccc7ccccc7c6)c6oc7ccccc7c6c5c4)nc(-c4cccc5oc6ccccc6c45)n3)ccc2c1 ; c1ccc(-c2cccc(-c3nc(-c4ccc5ccc6oc7c(-c8ccc9ccccc9c8)cccc7c6c5c4)nc(-c4cccc5oc6ccccc6c45)n3)c2)cc1]

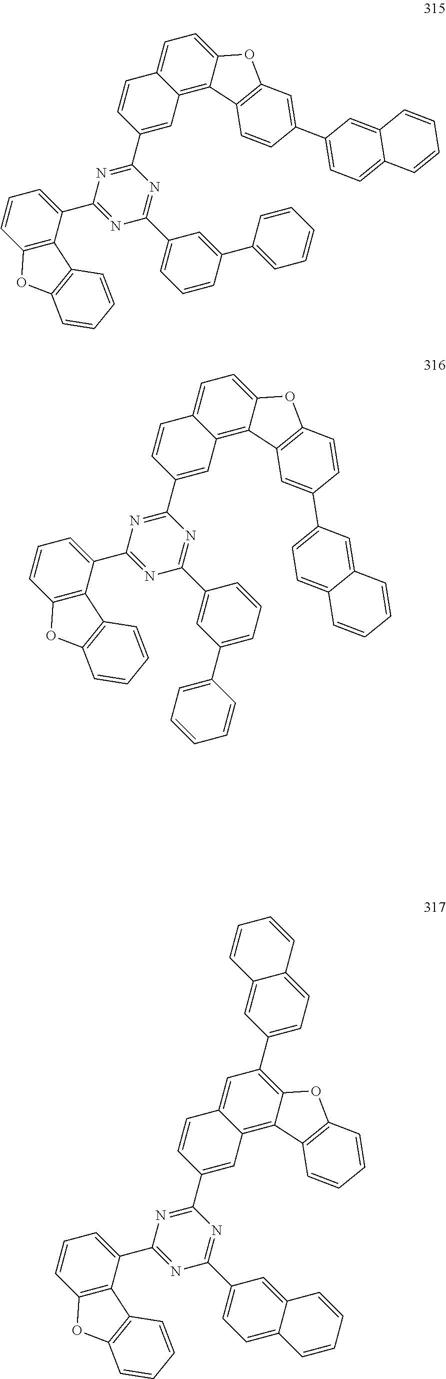 [SMILES: c1ccc(-c2cccc(-c3nc(-c4ccc5ccc6oc7cc(-c8ccc9ccccc9c8)ccc7c6c5c4)nc(-c4cccc5oc6ccccc6c45)n3)c2)cc1 ; c1ccc(-c2cccc(-c3nc(-c4ccc5ccc6oc7ccc(-c8ccc9ccccc9c8)cc7c6c5c4)nc(-c4cccc5oc6ccccc6c45)n3)c2)cc1 ; c1ccc2cc(-c3nc(-c4ccc5cc(-c6ccc7ccccc7c6)c6oc7ccccc7c6c5c4)nc(-c4cccc5oc6ccccc6c45)n3)ccc2c1]

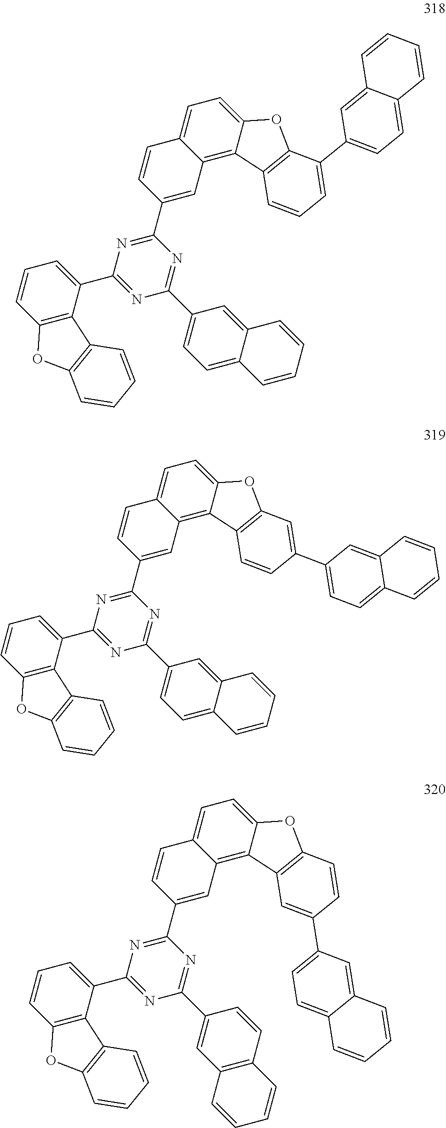 [SMILES: c1ccc2cc(-c3nc(-c4ccc5ccc6oc7c(-c8ccc9ccccc9c8)cccc7c6c5c4)nc(-c4cccc5oc6ccccc6c45)n3)ccc2c1 ; c1ccc2cc(-c3ccc4c(c3)oc3ccc5ccc(-c6nc(-c7ccc8ccccc8c7)nc(-c7cccc8oc9ccccc9c78)n6)cc5c34)ccc2c1 ; c1ccc2cc(-c3ccc4oc5ccc6ccc(-c7nc(-c8ccc9ccccc9c8)nc(-c8cccc9oc%10ccccc%10c89)n7)cc6c5c4c3)ccc2c1]

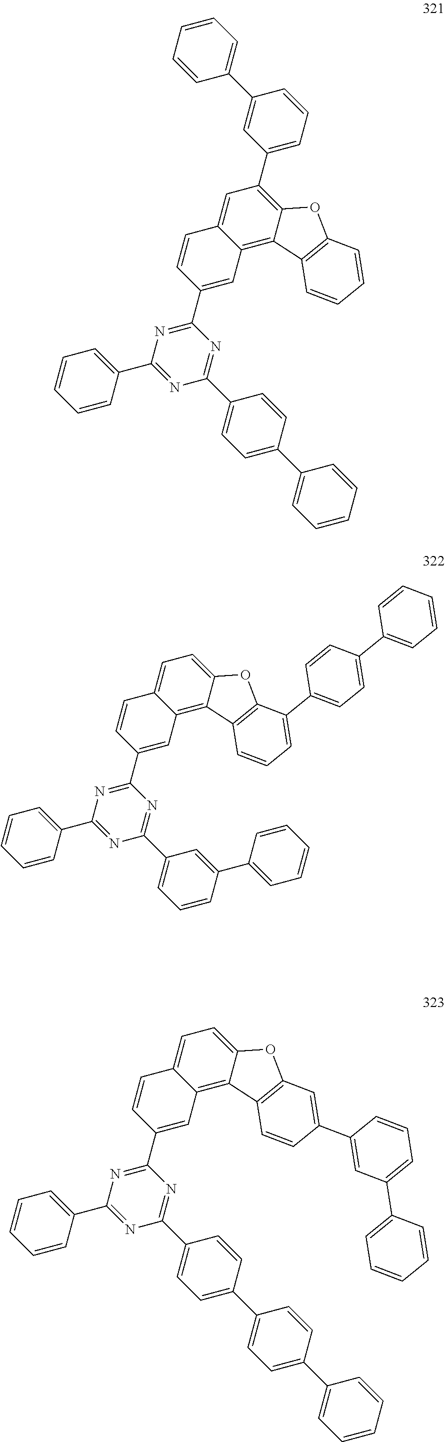 [SMILES: c1ccc(-c2ccc(-c3nc(-c4ccccc4)nc(-c4ccc5cc(-c6cccc(-c7ccccc7)c6)c6oc7ccccc7c6c5c4)n3)cc2)cc1 ; c1ccc(-c2ccc(-c3cccc4c3oc3ccc5ccc(-c6nc(-c7ccccc7)nc(-c7cccc(-c8ccccc8)c7)n6)cc5c34)cc2)cc1 ; c1ccc(-c2ccc(-c3ccc(-c4nc(-c5ccccc5)nc(-c5ccc6ccc7oc8cc(-c9cccc(-c%10ccccc%10)c9)ccc8c7c6c5)n4)cc3)cc2)cc1]

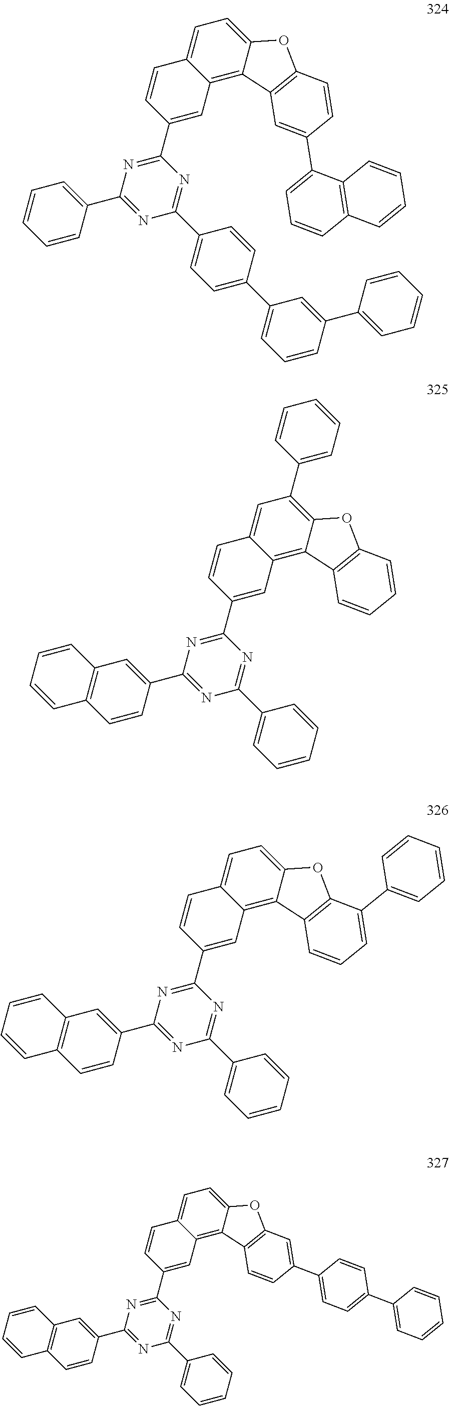

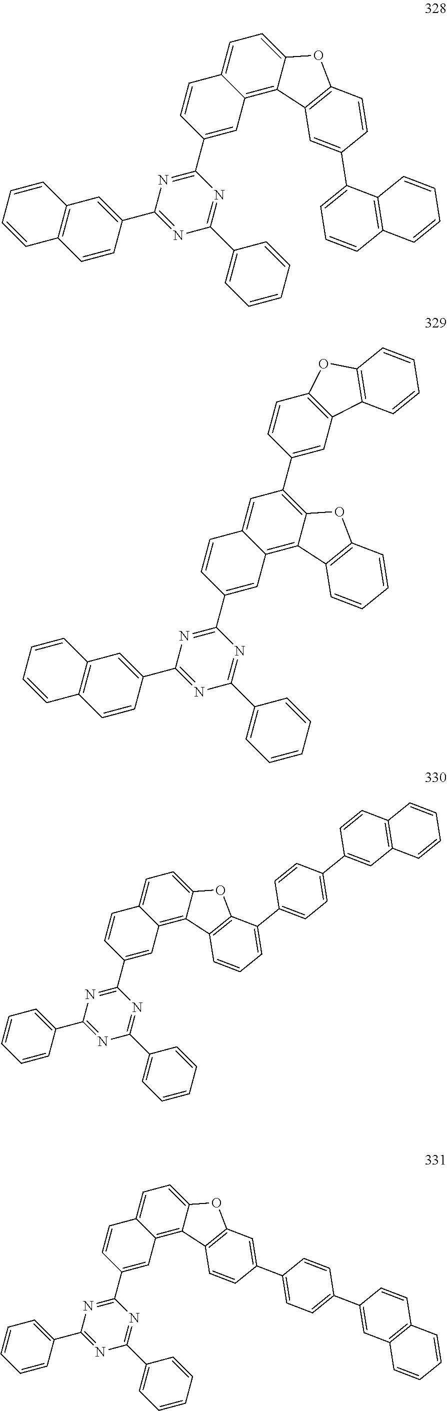 [SMILES: c1ccc(-c2nc(-c3ccc4ccccc4c3)nc(-c3ccc4ccc5oc6ccc(-c7cccc8ccccc78)cc6c5c4c3)n2)cc1 ; c1ccc(-c2nc(-c3ccc4ccccc4c3)nc(-c3ccc4cc(-c5ccc6oc7ccccc7c6c5)c5oc6ccccc6c5c4c3)n2)cc1 ; c1ccc(-c2nc(-c3ccccc3)nc(-c3ccc4ccc5oc6c(-c7ccc(-c8ccc9ccccc9c8)cc7)cccc6c5c4c3)n2)cc1 ; c1ccc(-c2nc(-c3ccccc3)nc(-c3ccc4ccc5oc6cc(-c7ccc(-c8ccc9ccccc9c8)cc7)ccc6c5c4c3)n2)cc1]

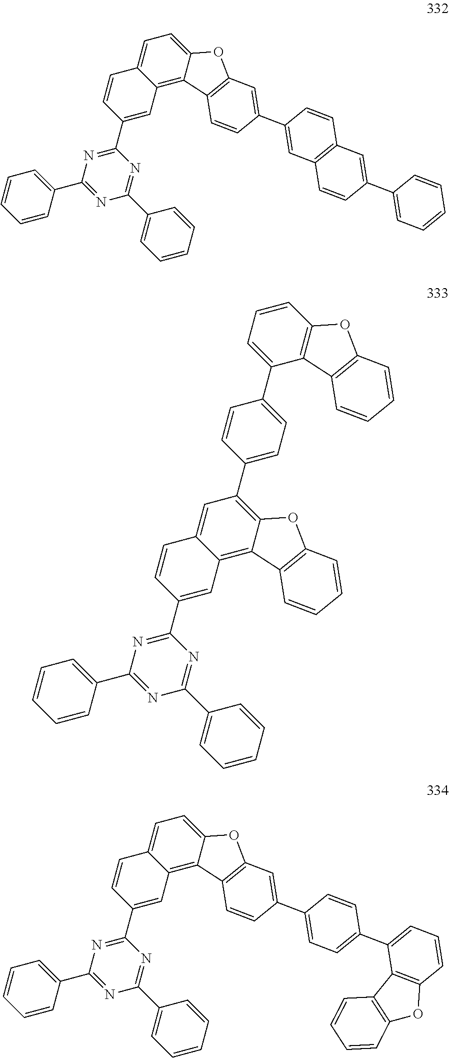 [SMILES: c1ccc(-c2ccc3cc(-c4ccc5c(c4)oc4ccc6ccc(-c7nc(-c8ccccc8)nc(-c8ccccc8)n7)cc6c45)ccc3c2)cc1 ; c1ccc(-c2nc(-c3ccccc3)nc(-c3ccc4cc(-c5ccc(-c6cccc7oc8ccccc8c67)cc5)c5oc6ccccc6c5c4c3)n2)cc1 ; c1ccc(-c2nc(-c3ccccc3)nc(-c3ccc4ccc5oc6cc(-c7ccc(-c8cccc9oc%10ccccc%10c89)cc7)ccc6c5c4c3)n2)cc1]

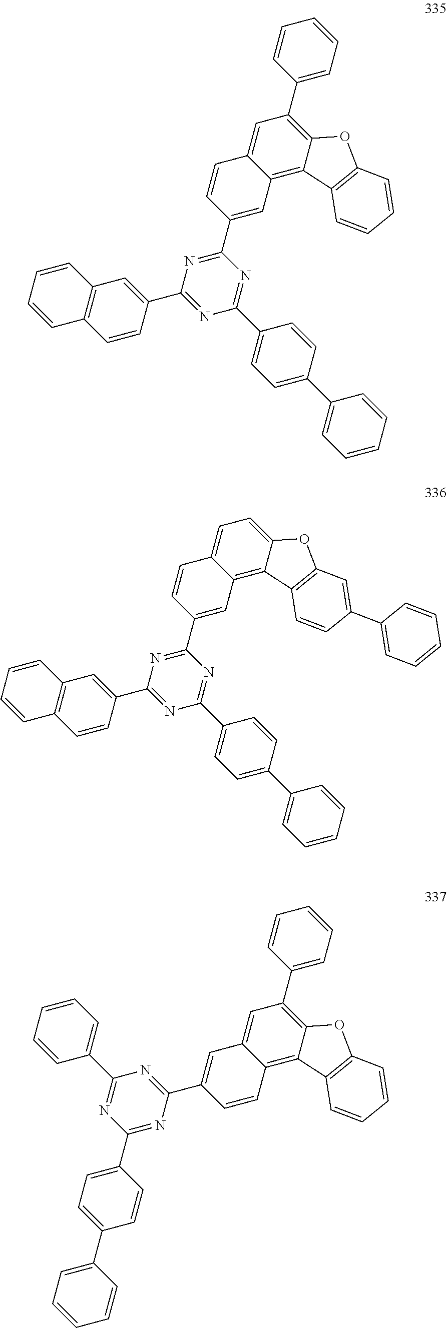 [SMILES: c1ccc(-c2ccc(-c3nc(-c4ccc5ccccc5c4)nc(-c4ccc5cc(-c6ccccc6)c6oc7ccccc7c6c5c4)n3)cc2)cc1 ; c1ccc(-c2ccc(-c3nc(-c4ccc5ccccc5c4)nc(-c4ccc5ccc6oc7cc(-c8ccccc8)ccc7c6c5c4)n3)cc2)cc1 ; c1ccc(-c2ccc(-c3nc(-c4ccccc4)nc(-c4ccc5c(c4)cc(-c4ccccc4)c4oc6ccccc6c45)n3)cc2)cc1]

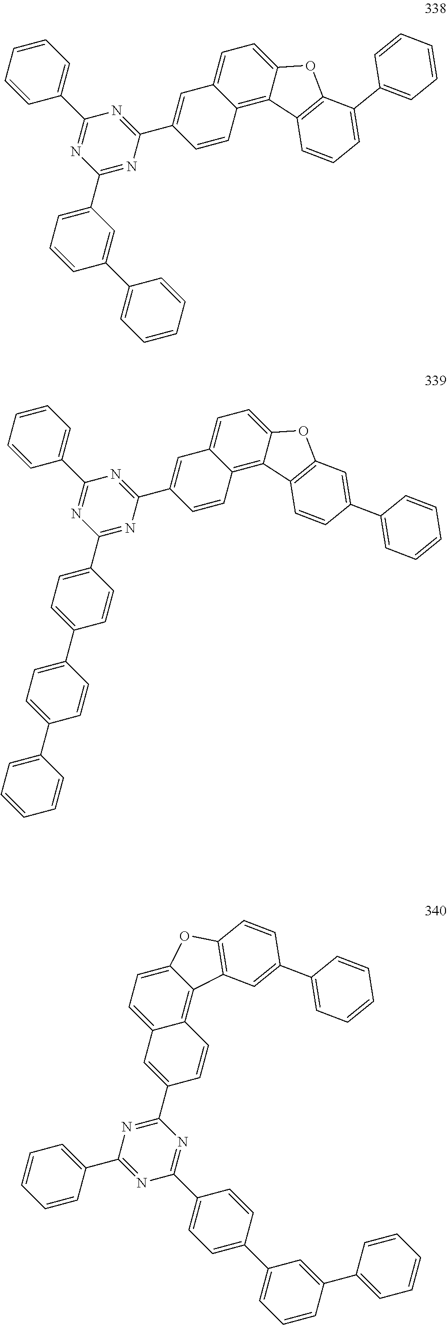 [SMILES: c1ccc(-c2cccc(-c3nc(-c4ccccc4)nc(-c4ccc5c(ccc6oc7c(-c8ccccc8)cccc7c65)c4)n3)c2)cc1 ; c1ccc(-c2ccc(-c3ccc(-c4nc(-c5ccccc5)nc(-c5ccc6c(ccc7oc8cc(-c9ccccc9)ccc8c76)c5)n4)cc3)cc2)cc1 ; c1ccc(-c2cccc(-c3ccc(-c4nc(-c5ccccc5)nc(-c5ccc6c(ccc7oc8ccc(-c9ccccc9)cc8c76)c5)n4)cc3)c2)cc1]

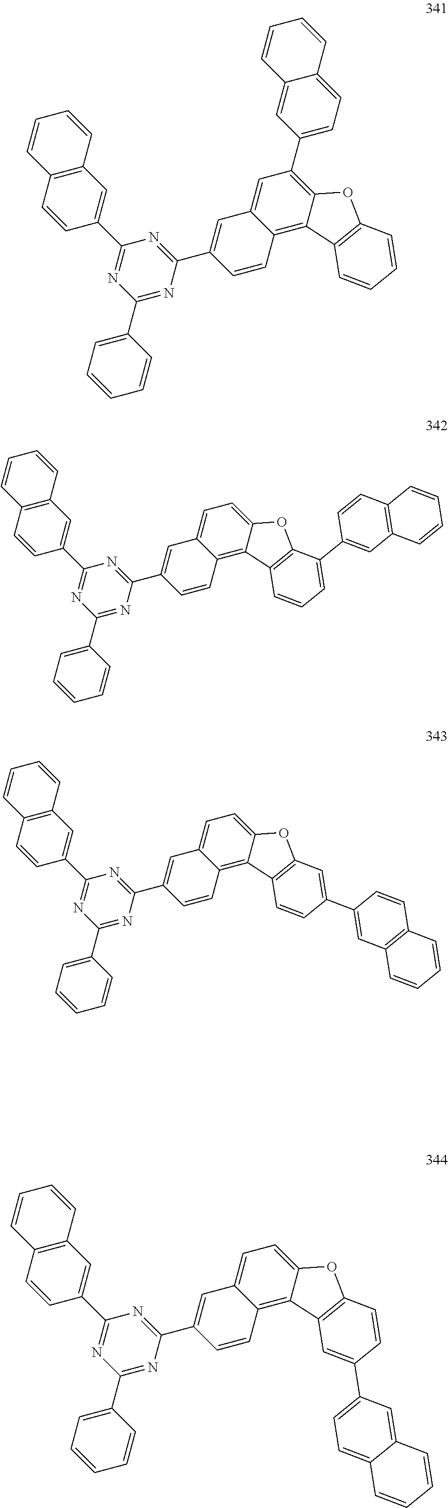 [SMILES: c1ccc(-c2nc(-c3ccc4ccccc4c3)nc(-c3ccc4c(c3)cc(-c3ccc5ccccc5c3)c3oc5ccccc5c34)n2)cc1 ; c1ccc(-c2nc(-c3ccc4ccccc4c3)nc(-c3ccc4c(ccc5oc6c(-c7ccc8ccccc8c7)cccc6c54)c3)n2)cc1 ; c1ccc(-c2nc(-c3ccc4ccccc4c3)nc(-c3ccc4c(ccc5oc6cc(-c7ccc8ccccc8c7)ccc6c54)c3)n2)cc1 ; c1ccc(-c2nc(-c3ccc4ccccc4c3)nc(-c3ccc4c(ccc5oc6ccc(-c7ccc8ccccc8c7)cc6c54)c3)n2)cc1]

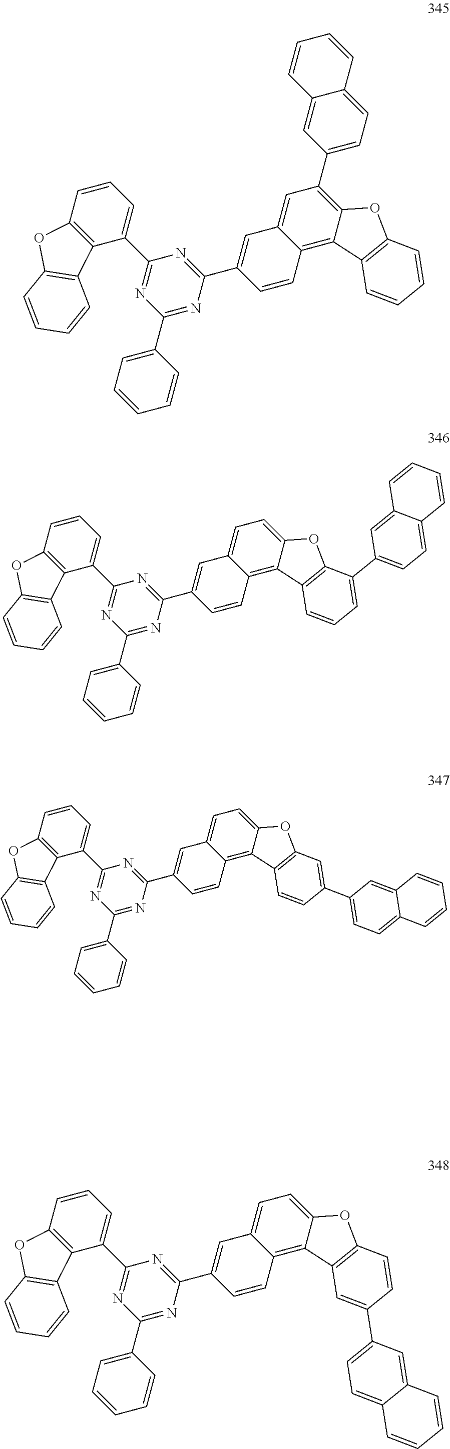 [SMILES: c1ccc(-c2nc(-c3ccc4c(c3)cc(-c3ccc5ccccc5c3)c3oc5ccccc5c34)nc(-c3cccc4oc5ccccc5c34)n2)cc1 ; c1ccc(-c2nc(-c3ccc4c(ccc5oc6c(-c7ccc8ccccc8c7)cccc6c54)c3)nc(-c3cccc4oc5ccccc5c34)n2)cc1 ; c1ccc(-c2nc(-c3ccc4c(ccc5oc6cc(-c7ccc8ccccc8c7)ccc6c54)c3)nc(-c3cccc4oc5ccccc5c34)n2)cc1 ; c1ccc(-c2nc(-c3ccc4c(ccc5oc6ccc(-c7ccc8ccccc8c7)cc6c54)c3)nc(-c3cccc4oc5ccccc5c34)n2)cc1]

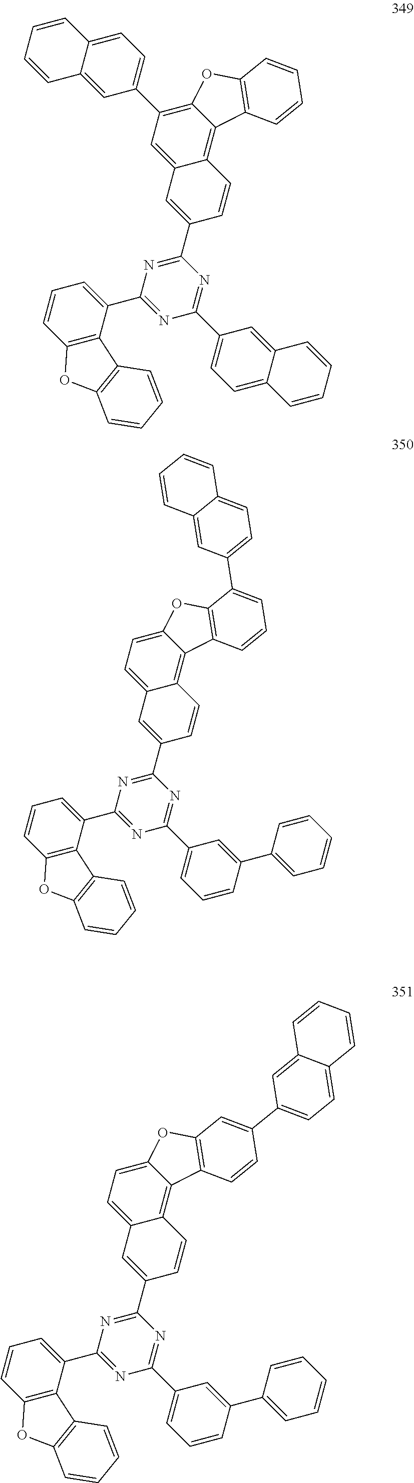 [SMILES: c1ccc2cc(-c3nc(-c4ccc5c(c4)cc(-c4ccc6ccccc6c4)c4oc6ccccc6c45)nc(-c4cccc5oc6ccccc6c45)n3)ccc2c1 ; c1ccc(-c2cccc(-c3nc(-c4ccc5c(ccc6oc7c(-c8ccc9ccccc9c8)cccc7c65)c4)nc(-c4cccc5oc6ccccc6c45)n3)c2)cc1 ; c1ccc(-c2cccc(-c3nc(-c4ccc5c(ccc6oc7cc(-c8ccc9ccccc9c8)ccc7c65)c4)nc(-c4cccc5oc6ccccc6c45)n3)c2)cc1]

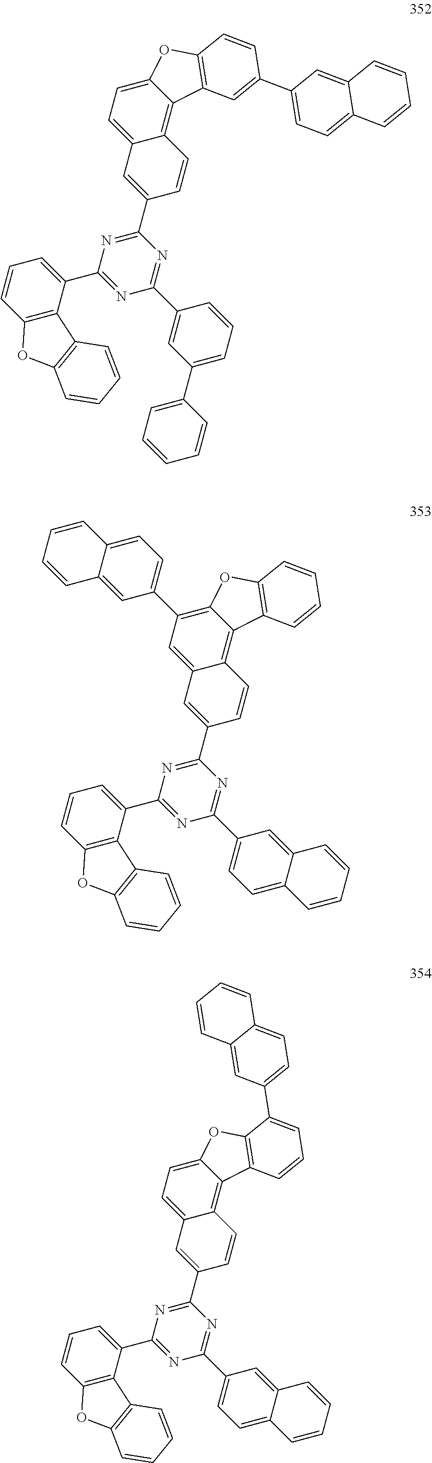 [SMILES: c1ccc(-c2cccc(-c3nc(-c4ccc5c(ccc6oc7ccc(-c8ccc9ccccc9c8)cc7c65)c4)nc(-c4cccc5oc6ccccc6c45)n3)c2)cc1 ; c1ccc2cc(-c3nc(-c4ccc5c(c4)cc(-c4ccc6ccccc6c4)c4oc6ccccc6c45)nc(-c4cccc5oc6ccccc6c45)n3)ccc2c1 ; c1ccc2cc(-c3nc(-c4ccc5c(ccc6oc7c(-c8ccc9ccccc9c8)cccc7c65)c4)nc(-c4cccc5oc6ccccc6c45)n3)ccc2c1]

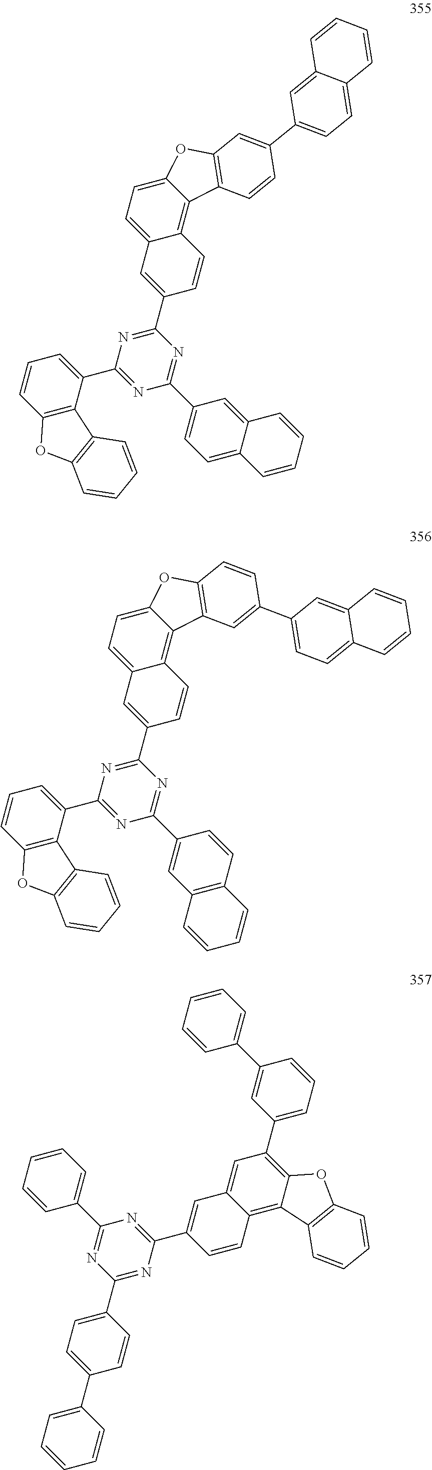

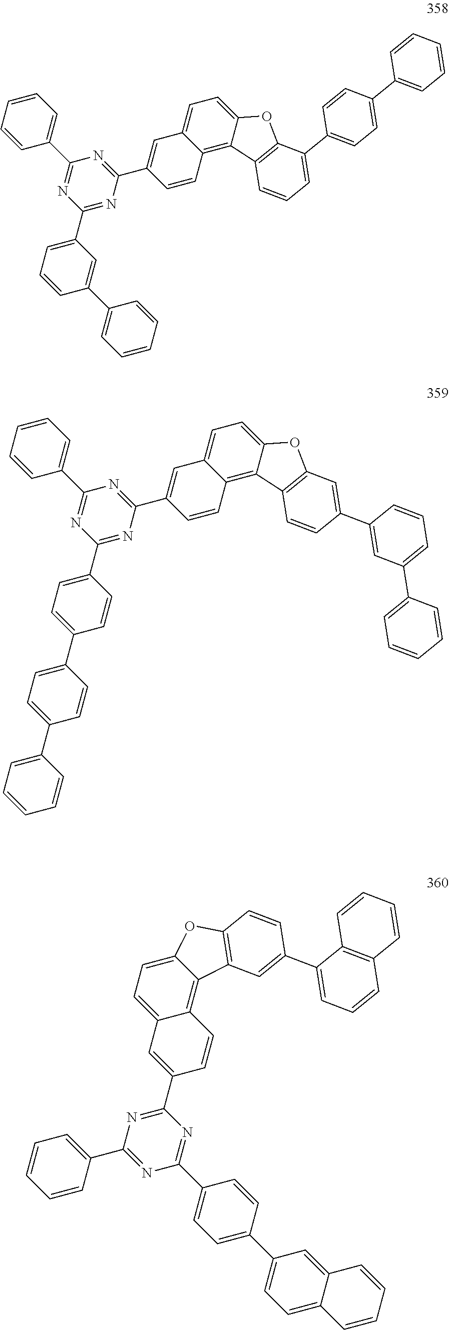 [SMILES: c1ccc(-c2ccc(-c3cccc4c3oc3ccc5cc(-c6nc(-c7ccccc7)nc(-c7cccc(-c8ccccc8)c7)n6)ccc5c34)cc2)cc1 ; c1ccc(-c2ccc(-c3ccc(-c4nc(-c5ccccc5)nc(-c5ccc6c(ccc7oc8cc(-c9cccc(-c%10ccccc%10)c9)ccc8c76)c5)n4)cc3)cc2)cc1 ; c1ccc(-c2nc(-c3ccc(-c4ccc5ccccc5c4)cc3)nc(-c3ccc4c(ccc5oc6ccc(-c7cccc8ccccc78)cc6c54)c3)n2)cc1]

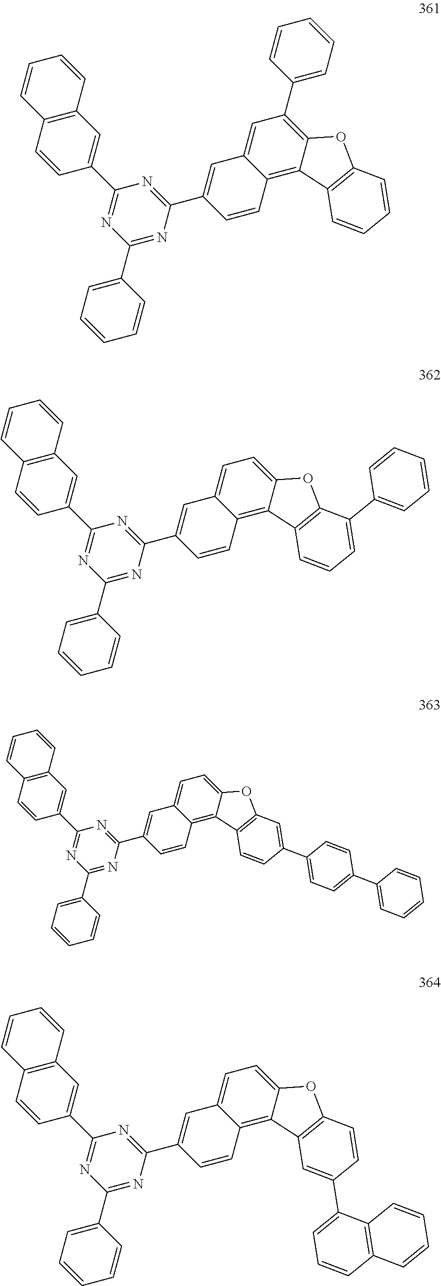 [SMILES: c1ccc(-c2nc(-c3ccc4ccccc4c3)nc(-c3ccc4c(c3)cc(-c3ccccc3)c3oc5ccccc5c34)n2)cc1 ; c1ccc(-c2nc(-c3ccc4ccccc4c3)nc(-c3ccc4c(ccc5oc6c(-c7ccccc7)cccc6c54)c3)n2)cc1 ; c1ccc(-c2ccc(-c3ccc4c(c3)oc3ccc5cc(-c6nc(-c7ccccc7)nc(-c7ccc8ccccc8c7)n6)ccc5c34)cc2)cc1 ; c1ccc(-c2nc(-c3ccc4ccccc4c3)nc(-c3ccc4c(ccc5oc6ccc(-c7cccc8ccccc78)cc6c54)c3)n2)cc1]

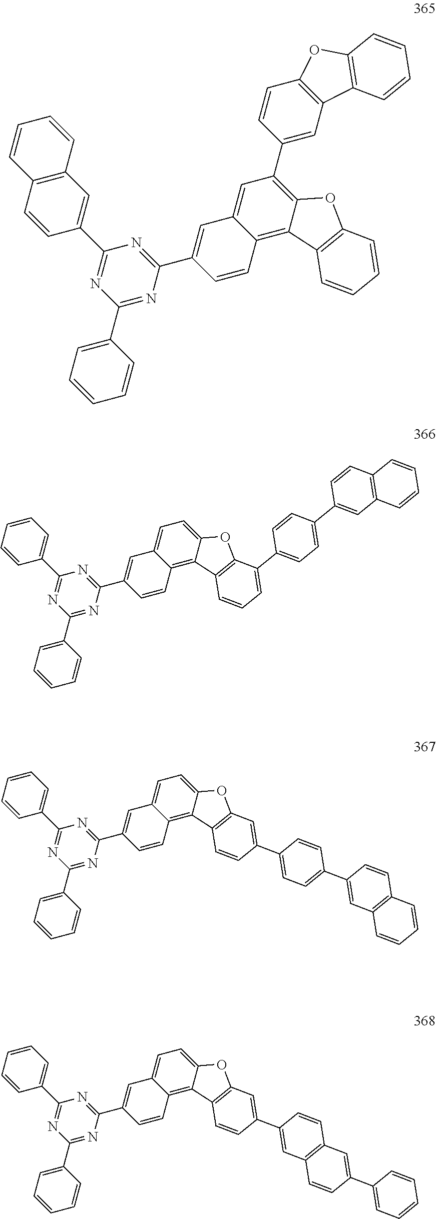 [SMILES: c1ccc(-c2nc(-c3ccc4ccccc4c3)nc(-c3ccc4c(c3)cc(-c3ccc5oc6ccccc6c5c3)c3oc5ccccc5c34)n2)cc1 ; c1ccc(-c2nc(-c3ccccc3)nc(-c3ccc4c(ccc5oc6c(-c7ccc(-c8ccc9ccccc9c8)cc7)cccc6c54)c3)n2)cc1 ; c1ccc(-c2nc(-c3ccccc3)nc(-c3ccc4c(ccc5oc6cc(-c7ccc(-c8ccc9ccccc9c8)cc7)ccc6c54)c3)n2)cc1 ; c1ccc(-c2ccc3cc(-c4ccc5c(c4)oc4ccc6cc(-c7nc(-c8ccccc8)nc(-c8ccccc8)n7)ccc6c45)ccc3c2)cc1]

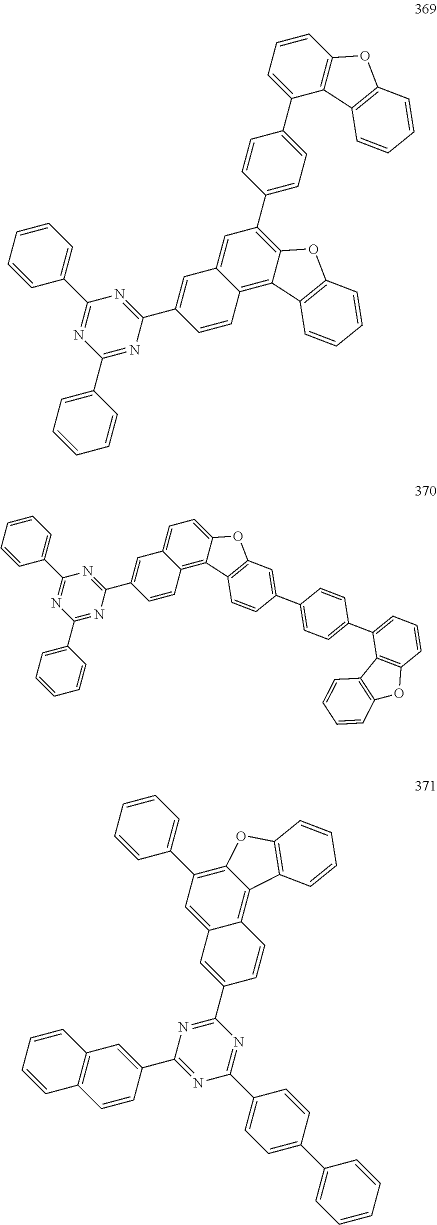 [SMILES: c1ccc(-c2nc(-c3ccccc3)nc(-c3ccc4c(c3)cc(-c3ccc(-c5cccc6oc7ccccc7c56)cc3)c3oc5ccccc5c34)n2)cc1 ; c1ccc(-c2nc(-c3ccccc3)nc(-c3ccc4c(ccc5oc6cc(-c7ccc(-c8cccc9oc%10ccccc%10c89)cc7)ccc6c54)c3)n2)cc1 ; c1ccc(-c2ccc(-c3nc(-c4ccc5ccccc5c4)nc(-c4ccc5c(c4)cc(-c4ccccc4)c4oc6ccccc6c45)n3)cc2)cc1]

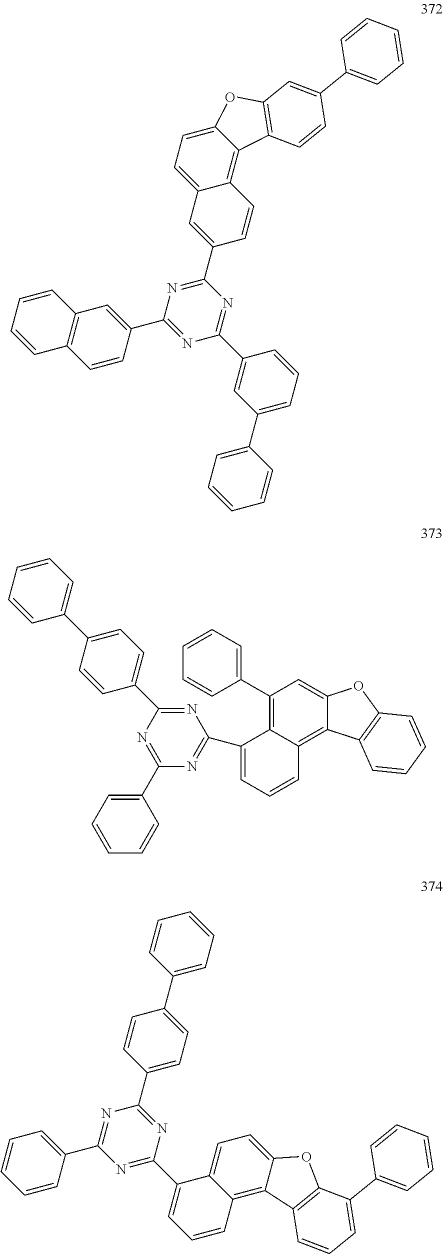 [SMILES: c1ccc(-c2cccc(-c3nc(-c4ccc5ccccc5c4)nc(-c4ccc5c(ccc6oc7cc(-c8ccccc8)ccc7c65)c4)n3)c2)cc1 ; c1ccc(-c2ccc(-c3nc(-c4ccccc4)nc(-c4cccc5c4c(-c4ccccc4)cc4oc6ccccc6c45)n3)cc2)cc1 ; c1ccc(-c2ccc(-c3nc(-c4ccccc4)nc(-c4cccc5c4ccc4oc6c(-c7ccccc7)cccc6c45)n3)cc2)cc1]

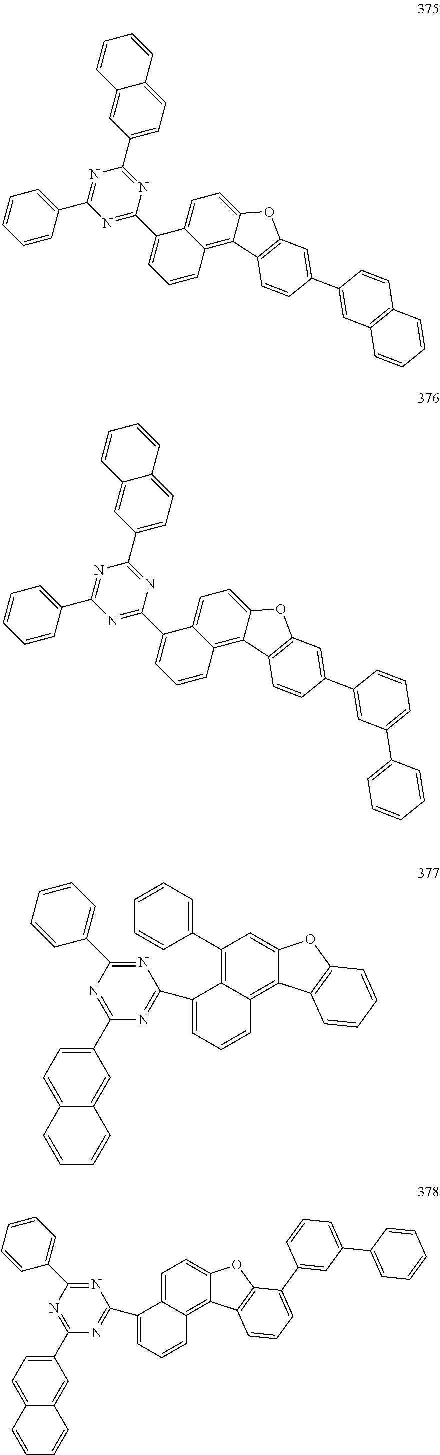 [SMILES: c1ccc(-c2nc(-c3ccc4ccccc4c3)nc(-c3cccc4c3ccc3oc5cc(-c6ccc7ccccc7c6)ccc5c34)n2)cc1 ; c1ccc(-c2cccc(-c3ccc4c(c3)oc3ccc5c(-c6nc(-c7ccccc7)nc(-c7ccc8ccccc8c7)n6)cccc5c34)c2)cc1 ; c1ccc(-c2nc(-c3ccc4ccccc4c3)nc(-c3cccc4c3c(-c3ccccc3)cc3oc5ccccc5c34)n2)cc1 ; c1ccc(-c2cccc(-c3cccc4c3oc3ccc5c(-c6nc(-c7ccccc7)nc(-c7ccc8ccccc8c7)n6)cccc5c34)c2)cc1]

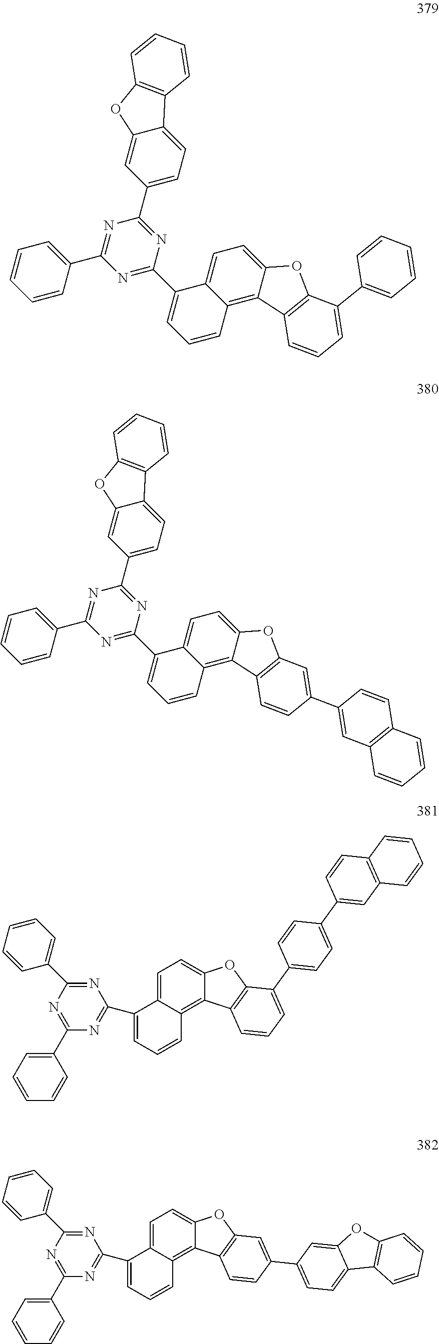 [SMILES: c1ccc(-c2nc(-c3ccc4c(c3)oc3ccccc34)nc(-c3cccc4c3ccc3oc5c(-c6ccccc6)cccc5c34)n2)cc1 ; c1ccc(-c2nc(-c3ccc4c(c3)oc3ccccc34)nc(-c3cccc4c3ccc3oc5cc(-c6ccc7ccccc7c6)ccc5c34)n2)cc1 ; c1ccc(-c2nc(-c3ccccc3)nc(-c3cccc4c3ccc3oc5c(-c6ccc(-c7ccc8ccccc8c7)cc6)cccc5c34)n2)cc1 ; c1ccc(-c2nc(-c3ccccc3)nc(-c3cccc4c3ccc3oc5cc(-c6ccc7c(c6)oc6ccccc67)ccc5c34)n2)cc1]

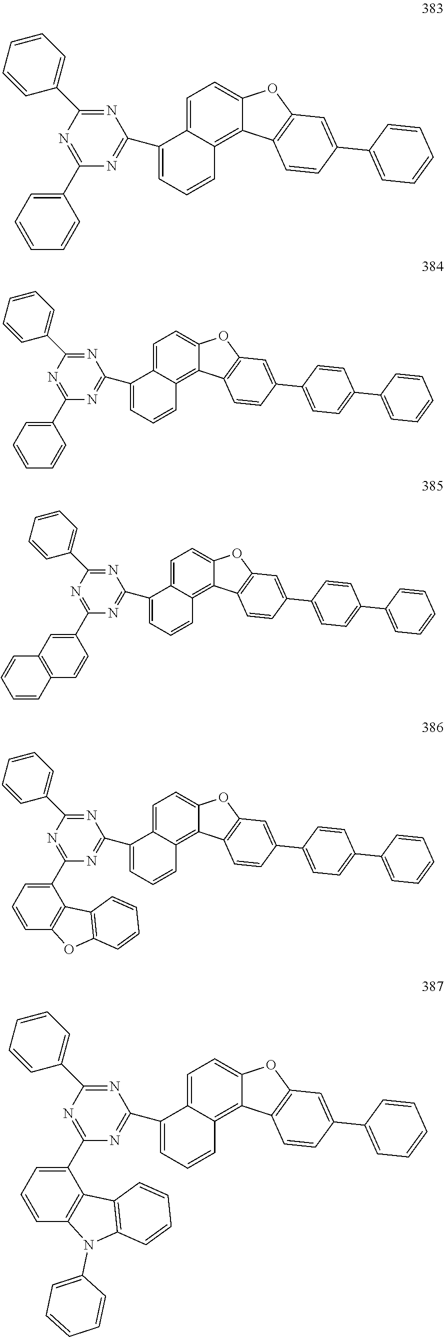 [SMILES: c1ccc(-c2ccc3c(c2)oc2ccc4c(-c5nc(-c6ccccc6)nc(-c6ccccc6)n5)cccc4c23)cc1 ; c1ccc(-c2ccc(-c3ccc4c(c3)oc3ccc5c(-c6nc(-c7ccccc7)nc(-c7ccccc7)n6)cccc5c34)cc2)cc1 ; c1ccc(-c2ccc(-c3ccc4c(c3)oc3ccc5c(-c6nc(-c7ccccc7)nc(-c7ccc8ccccc8c7)n6)cccc5c34)cc2)cc1 ; c1ccc(-c2ccc(-c3ccc4c(c3)oc3ccc5c(-c6nc(-c7ccccc7)nc(-c7cccc8oc9ccccc9c78)n6)cccc5c34)cc2)cc1 ; c1ccc(-c2ccc3c(c2)oc2ccc4c(-c5nc(-c6ccccc6)nc(-c6cccc7c6c6ccccc6n7-c6ccccc6)n5)cccc4c23)cc1]

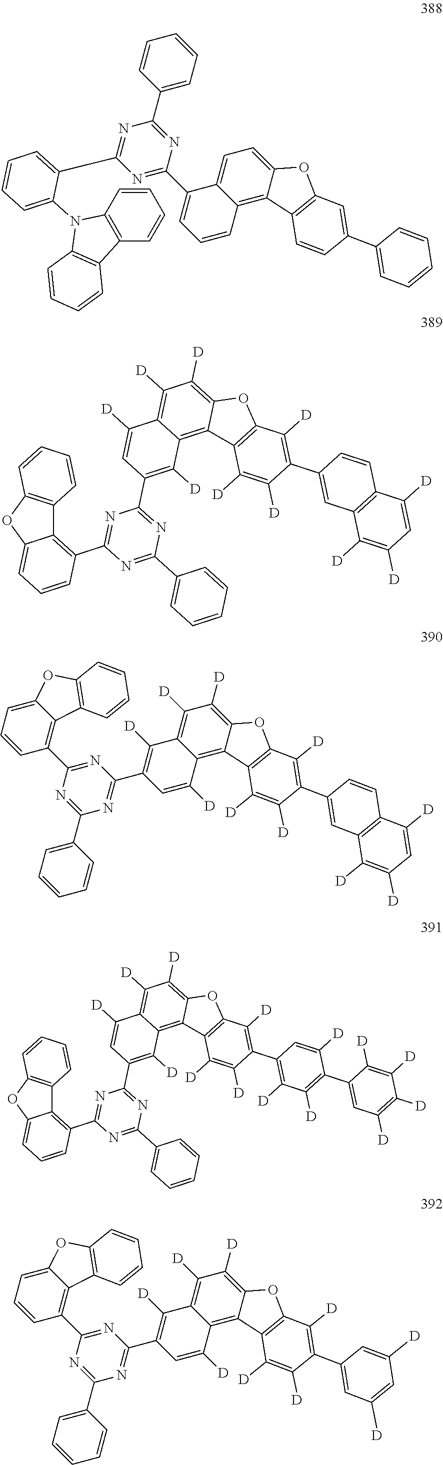

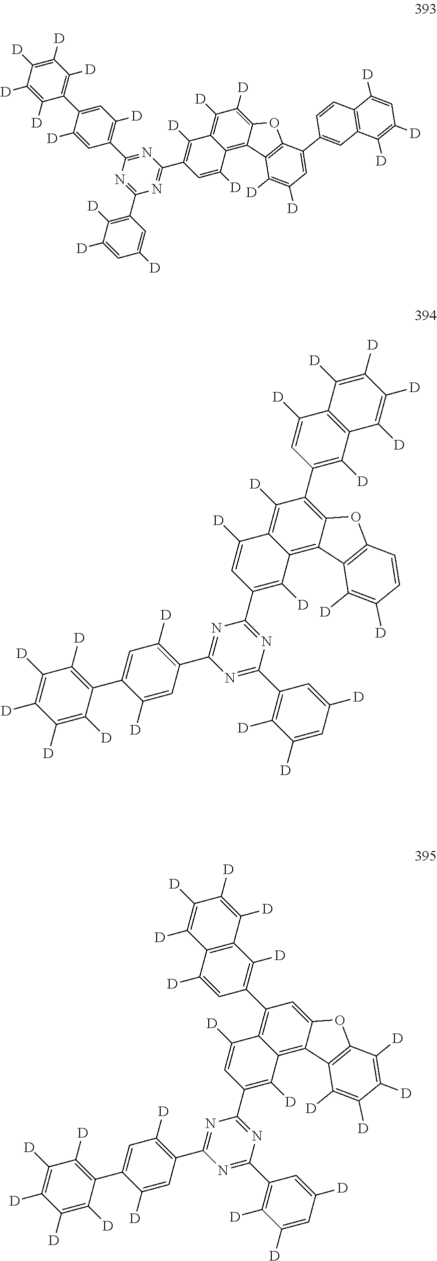

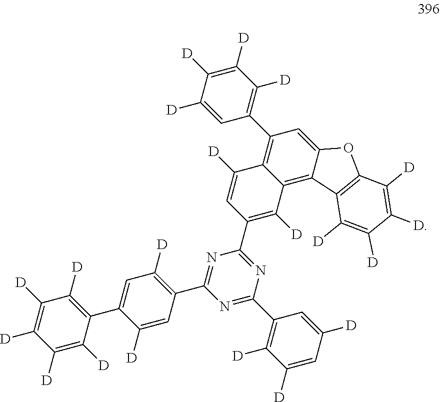

9. A light-emitting device comprising:
a first electrode;
a second electrode disposed on the first electrode; and
one or more organic layers interposed between the first electrode and the second electrode,
wherein one or more of the organic layers comprises the heterocyclic compound according to claim 1.

10. The light-emitting device according to claim 9, wherein the organic layer comprises a light-emitting layer, and
the light-emitting layer comprises a dopant material and a host material, and the host material comprises the heterocyclic compound.

11. The light-emitting device according to claim 10, wherein the organic layer further comprises a hole transport layer disposed between the first electrode and the light-emitting layer, and an electron transport layer disposed between the light-emitting layer and the second electrode.

12. The light-emitting device according to claim 10, wherein the host material further comprises a compound represented by Formula 3: 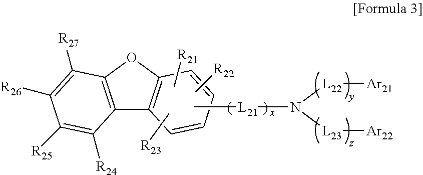
wherein, in Formula 3, two adjacent groups of R₂₁ to R₂₇ are linked to each other to form an aromatic ring represented by 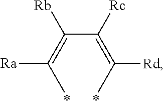
any one of the remainders of R₂₁ to R₂₇ that do not form a ring is a substituted or unsubstituted aryl group having 6 to 60 carbon atoms, or a substituted or unsubstituted heteroaryl group having 2 to 60 carbon atoms, and
Ra, Rb, Rc, Rd, and the remaining substituents are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, or adjacent groups are linked to form a substituted or unsubstituted 5- to 9-membered saturated or unsaturated ring,
L₂₁ to L₂₃ are each independently a direct linkage, a substituted or unsubstituted arylene group having 6 to 60 carbon atoms, or a substituted or unsubstituted heteroarylene group having 2 to 60 carbon atoms,
x, y, and z are each independently 0 or an integer from 1 to 5,
when x is an integer from 2 to 5, a plurality of L₂₁ groups are the same or different,
when y is an integer from 2 to 5, a plurality of L₂₂ groups are the same or different,
when z is an integer from 2 to 5, a plurality of L₂₃ groups are the same or different, and
Ar₂₁ and Ar₂₂ are each independently a substituted or unsubstituted aryl group having 6 to 60 carbon atoms, or a substituted or unsubstituted heteroaryl group having 2 to 60 carbon atoms.

13. The light-emitting device according to claim 12, wherein the deuterium content of the compound represented by Formula 3 is 0%, or greater than 0% and not more than 100%.

14. The light-emitting device according to claim 12, wherein the compound represented by Formula 3 is represented by at least one selected from formulae below:

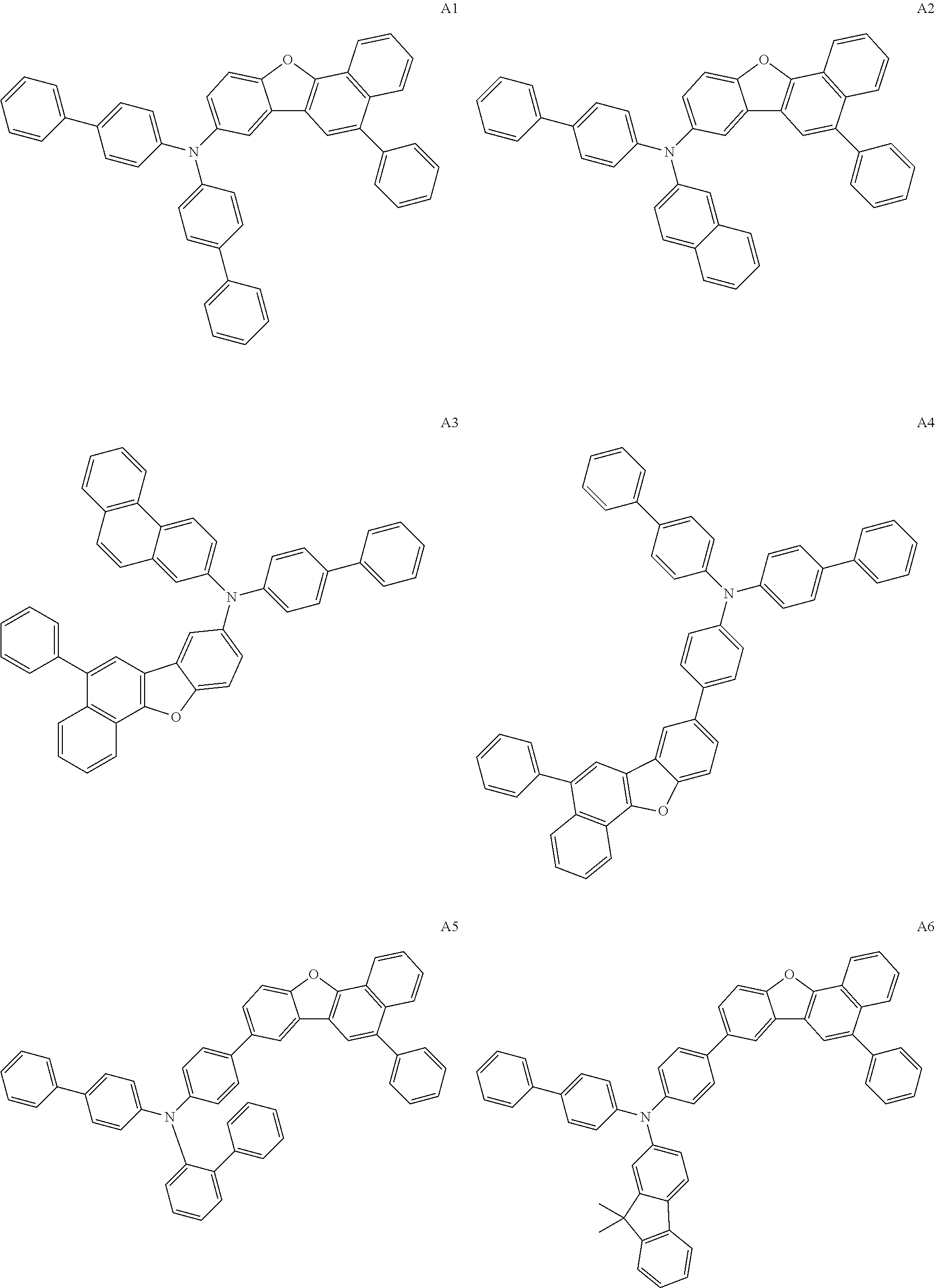

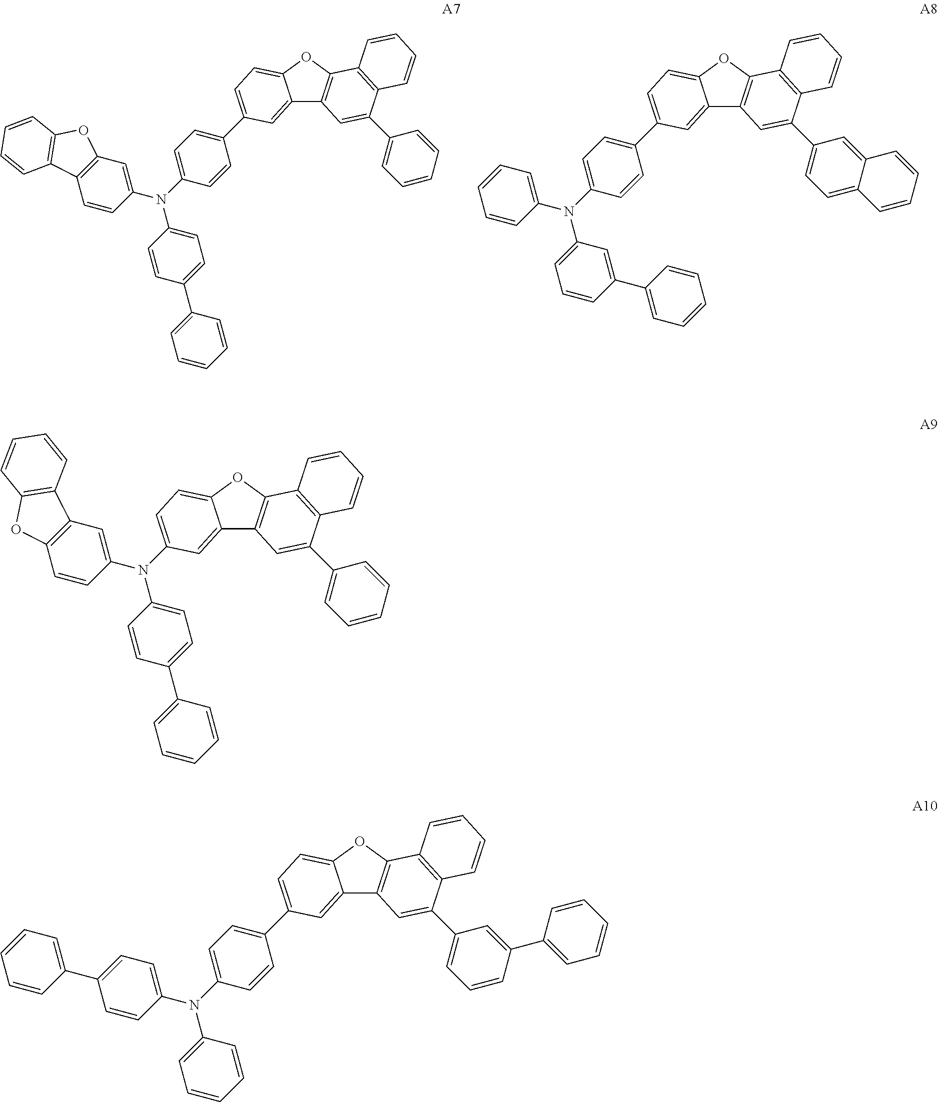 [SMILES: c1ccc(-c2ccc(N(c3ccc(-c4ccc5oc6c7ccccc7c(-c7ccccc7)cc6c5c4)cc3)c3ccc4c(c3)oc3ccccc34)cc2)cc1 ; c1ccc(-c2cccc(N(c3ccccc3)c3ccc(-c4ccc5oc6c7ccccc7c(-c7ccc8ccccc8c7)cc6c5c4)cc3)c2)cc1 ; c1ccc(-c2ccc(N(c3ccc4oc5ccccc5c4c3)c3ccc4oc5c6ccccc6c(-c6ccccc6)cc5c4c3)cc2)cc1 ; c1ccc(-c2ccc(N(c3ccccc3)c3ccc(-c4ccc5oc6c7ccccc7c(-c7cccc(-c8ccccc8)c7)cc6c5c4)cc3)cc2)cc1]

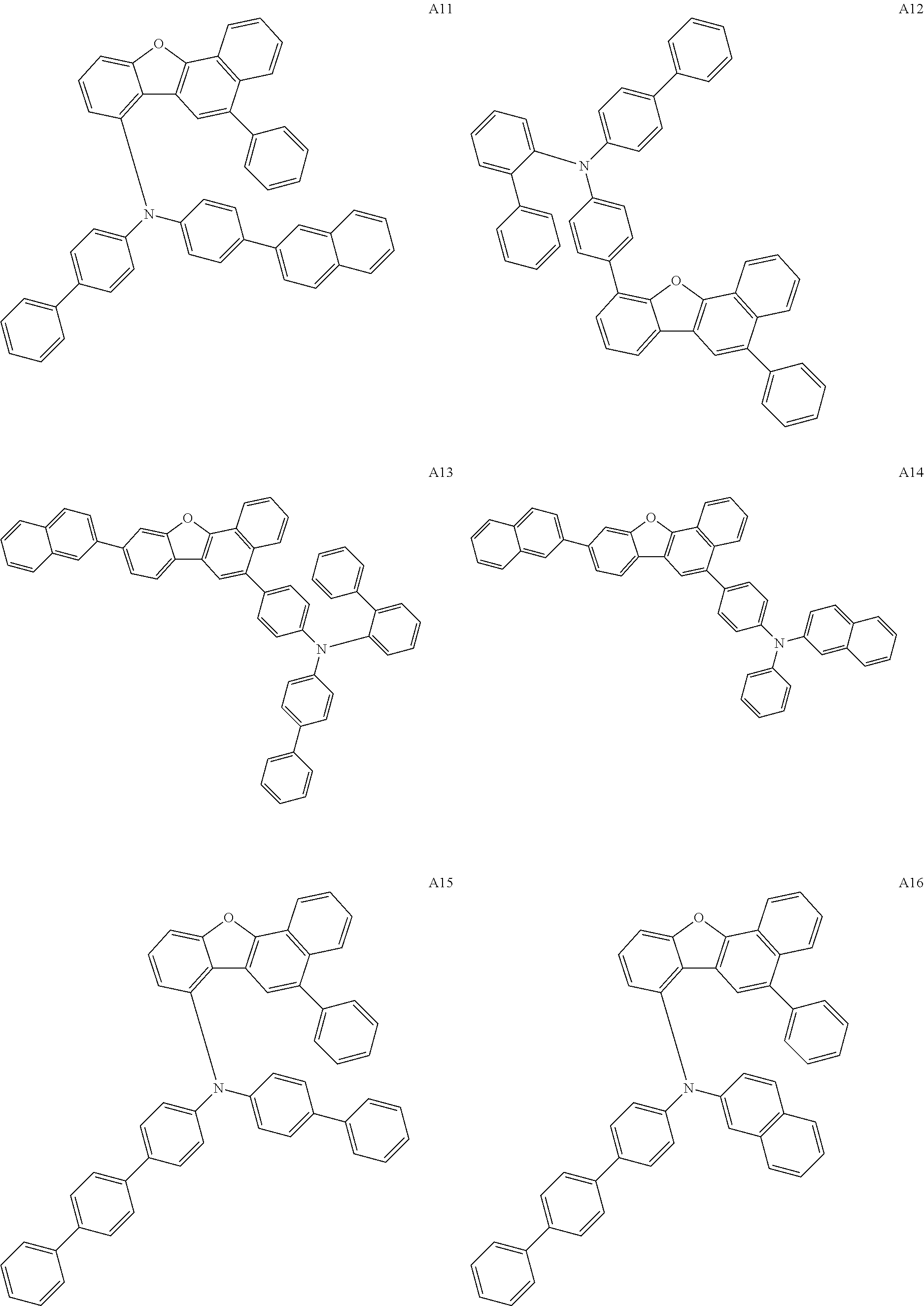

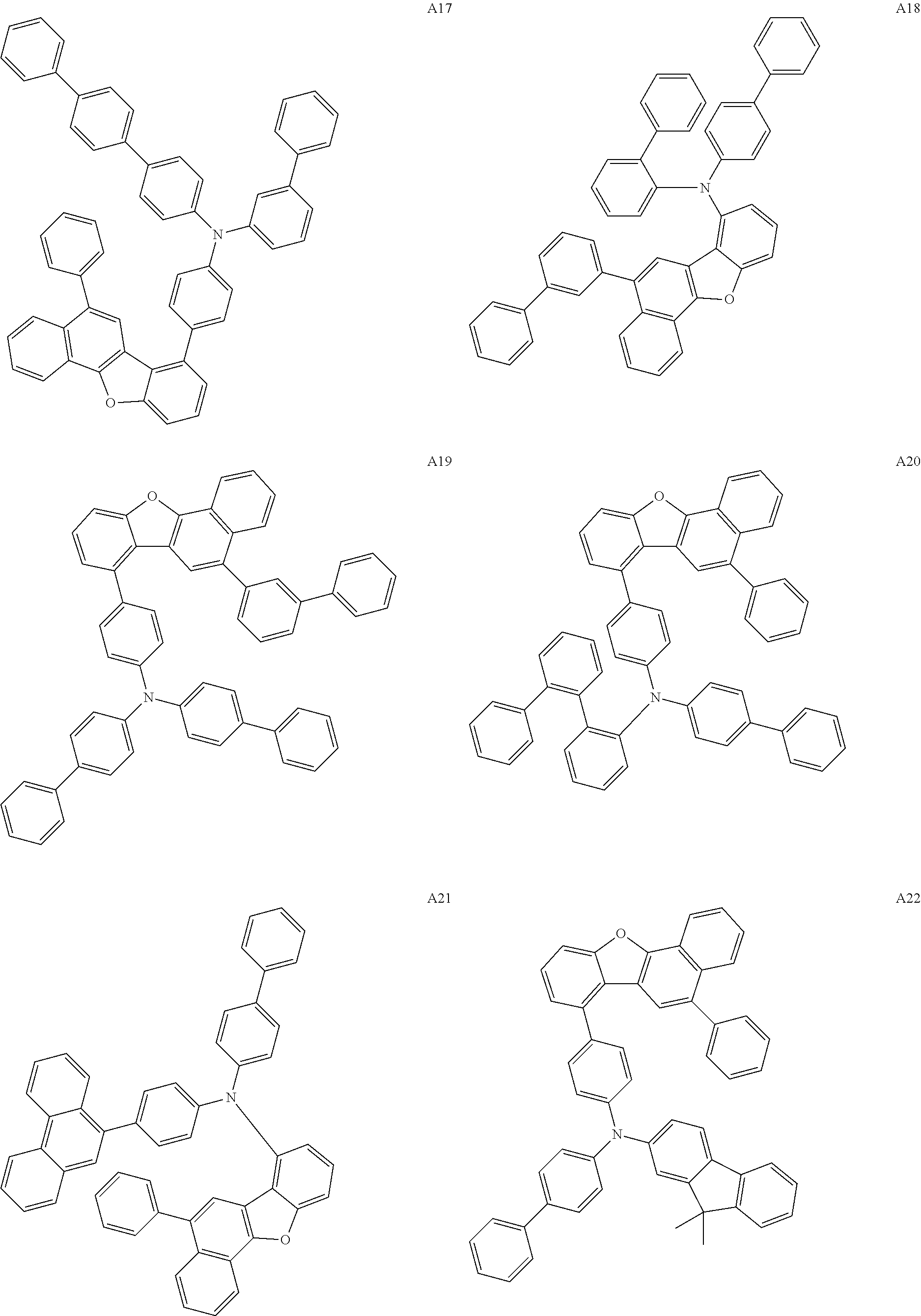

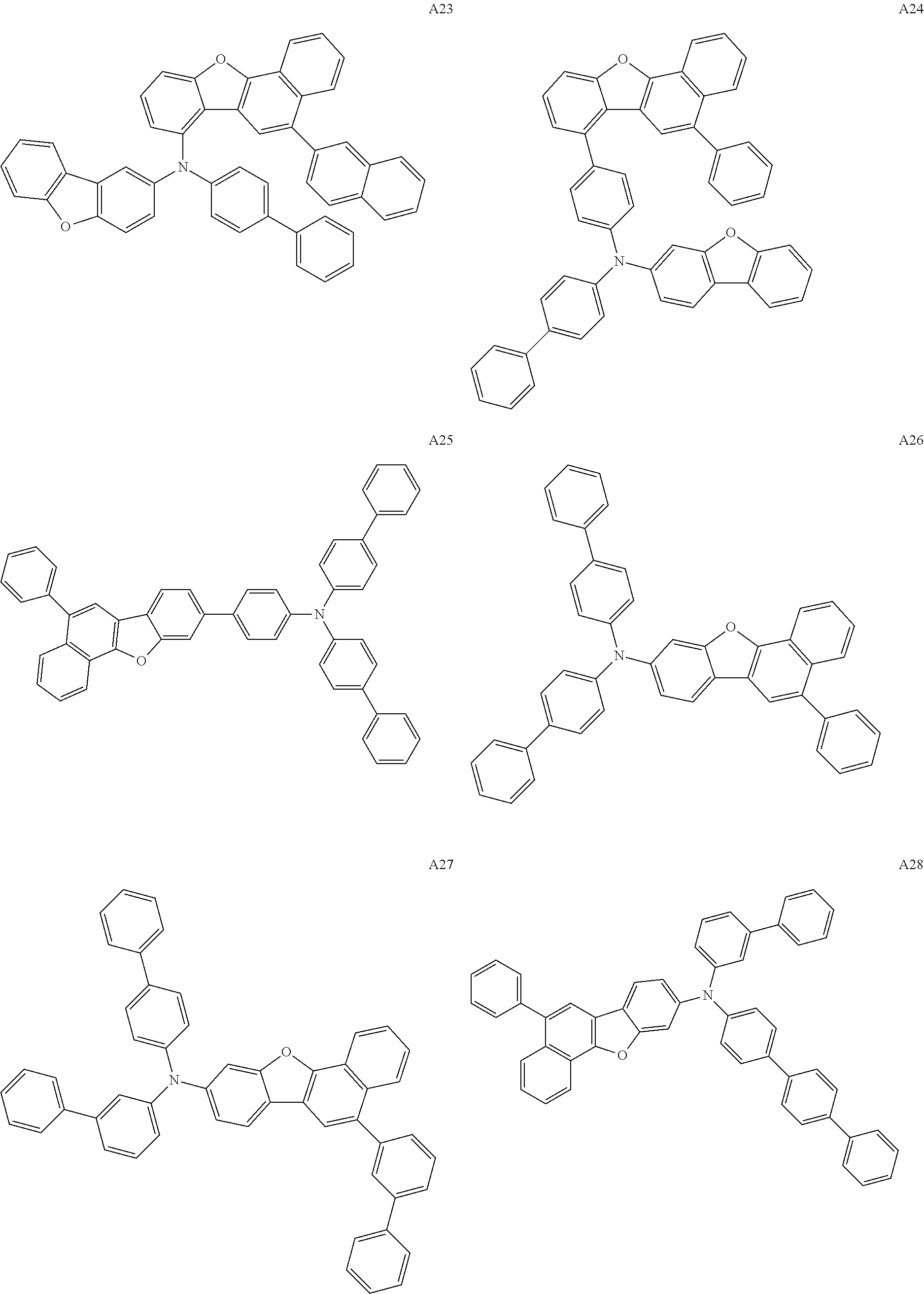

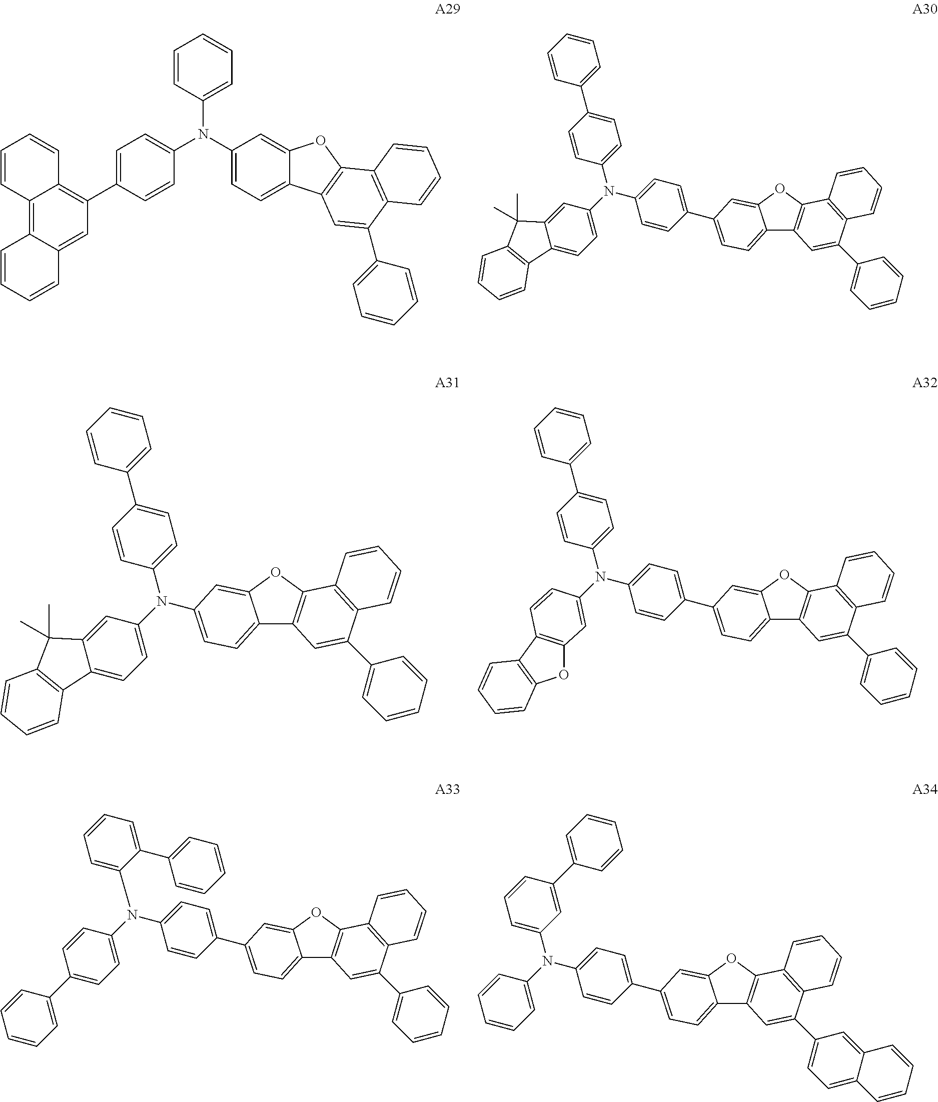

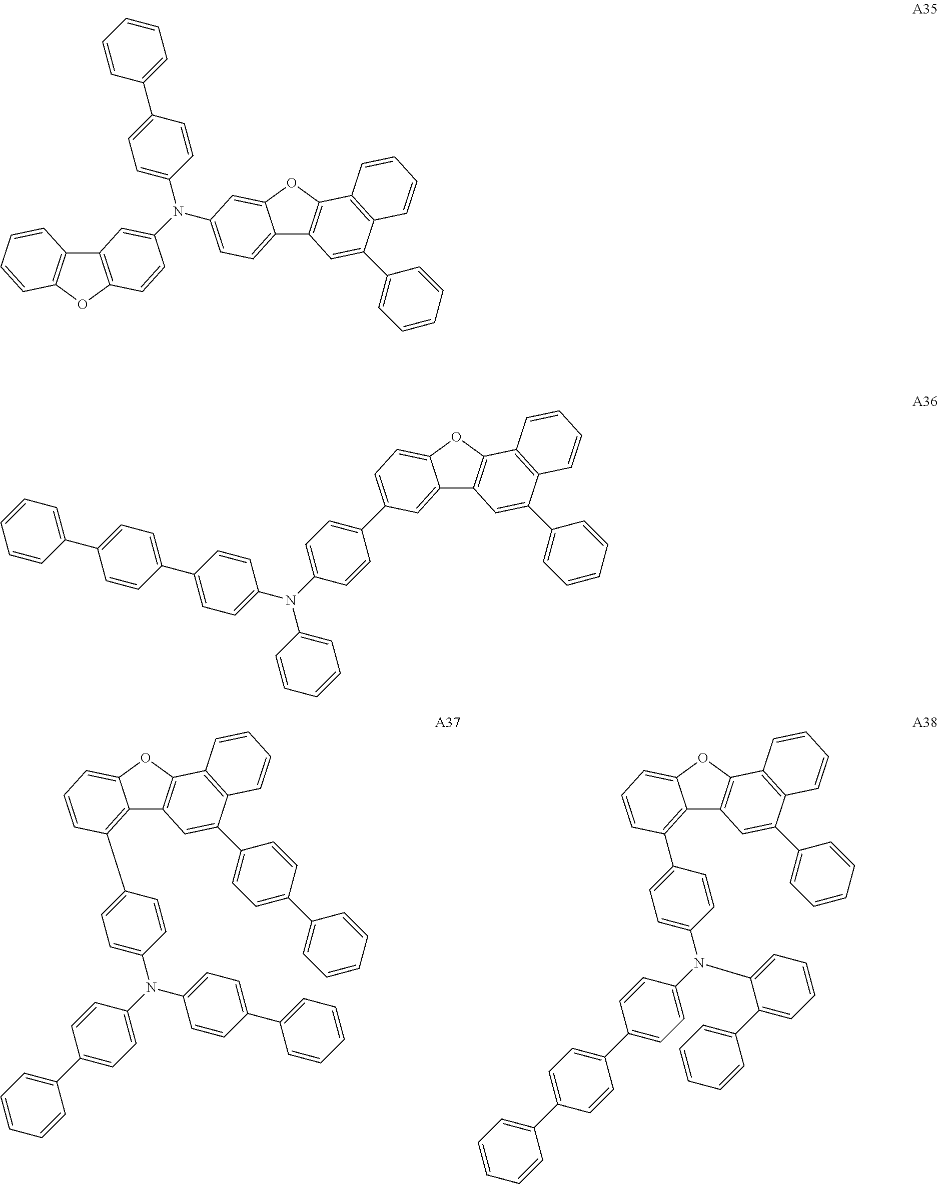 [SMILES: c1ccc(-c2ccc(N(c3ccc4c(c3)oc3c5ccccc5c(-c5ccccc5)cc43)c3ccc4oc5ccccc5c4c3)cc2)cc1 ; c1ccc(-c2ccc(-c3ccc(N(c4ccccc4)c4ccc(-c5ccc6oc7c8ccccc8c(-c8ccccc8)cc7c6c5)cc4)cc3)cc2)cc1 ; c1ccc(-c2ccc(-c3cc4c(oc5cccc(-c6ccc(N(c7ccc(-c8ccccc8)cc7)c7ccc(-c8ccccc8)cc7)cc6)c54)c4ccccc34)cc2)cc1 ; c1ccc(-c2ccc(-c3ccc(N(c4ccc(-c5cccc6oc7c8ccccc8c(-c8ccccc8)cc7c56)cc4)c4ccccc4-c4ccccc4)cc3)cc2)cc1]

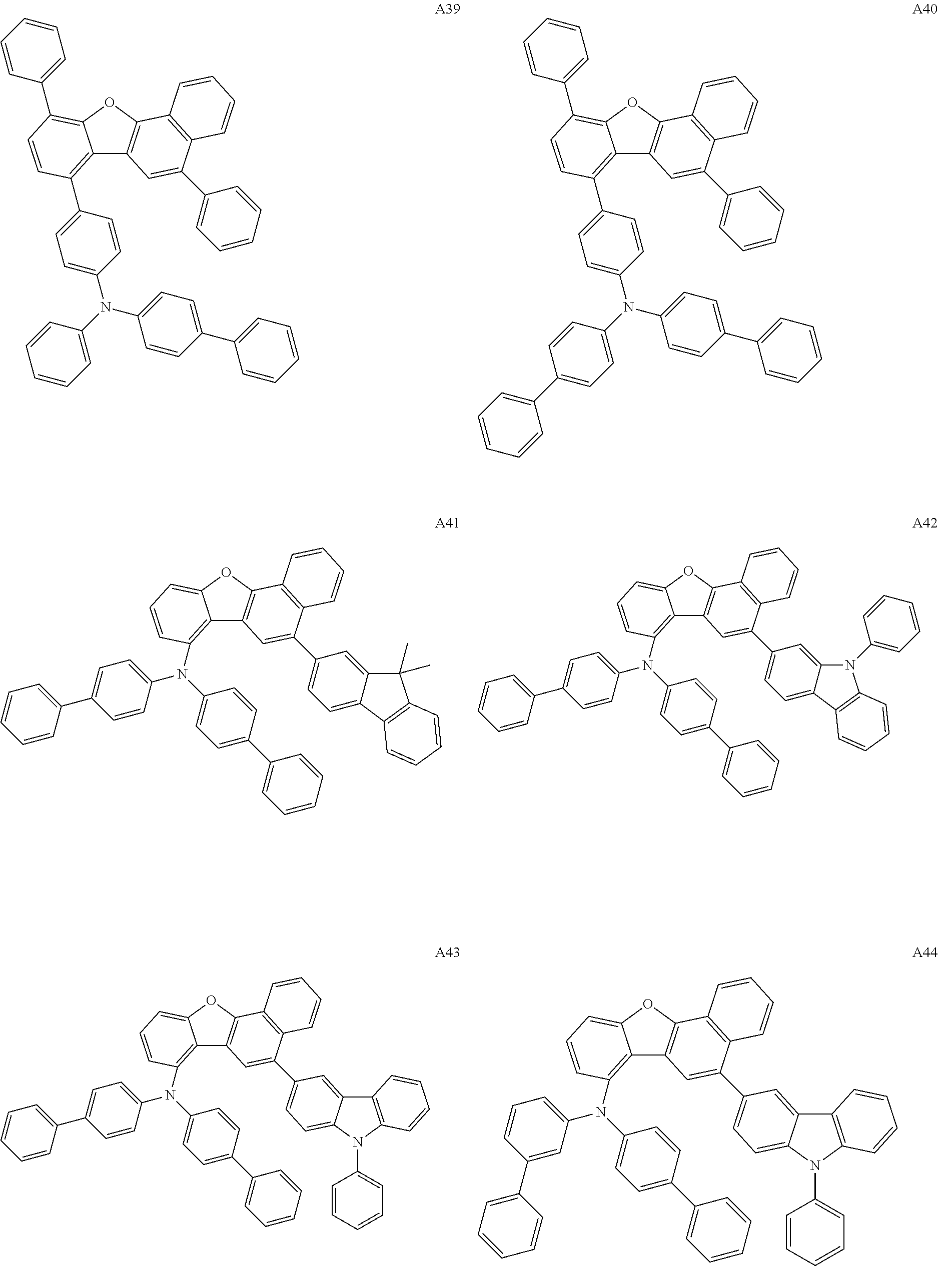

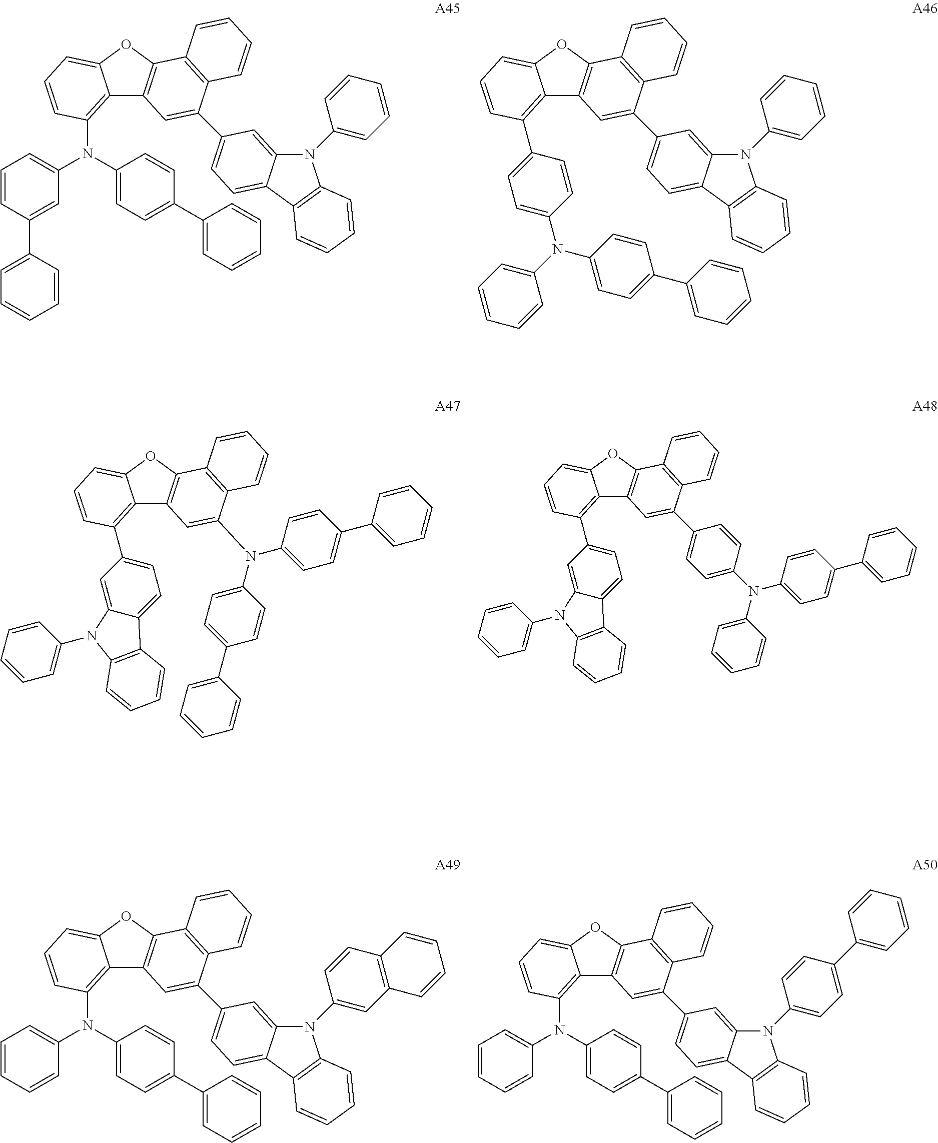

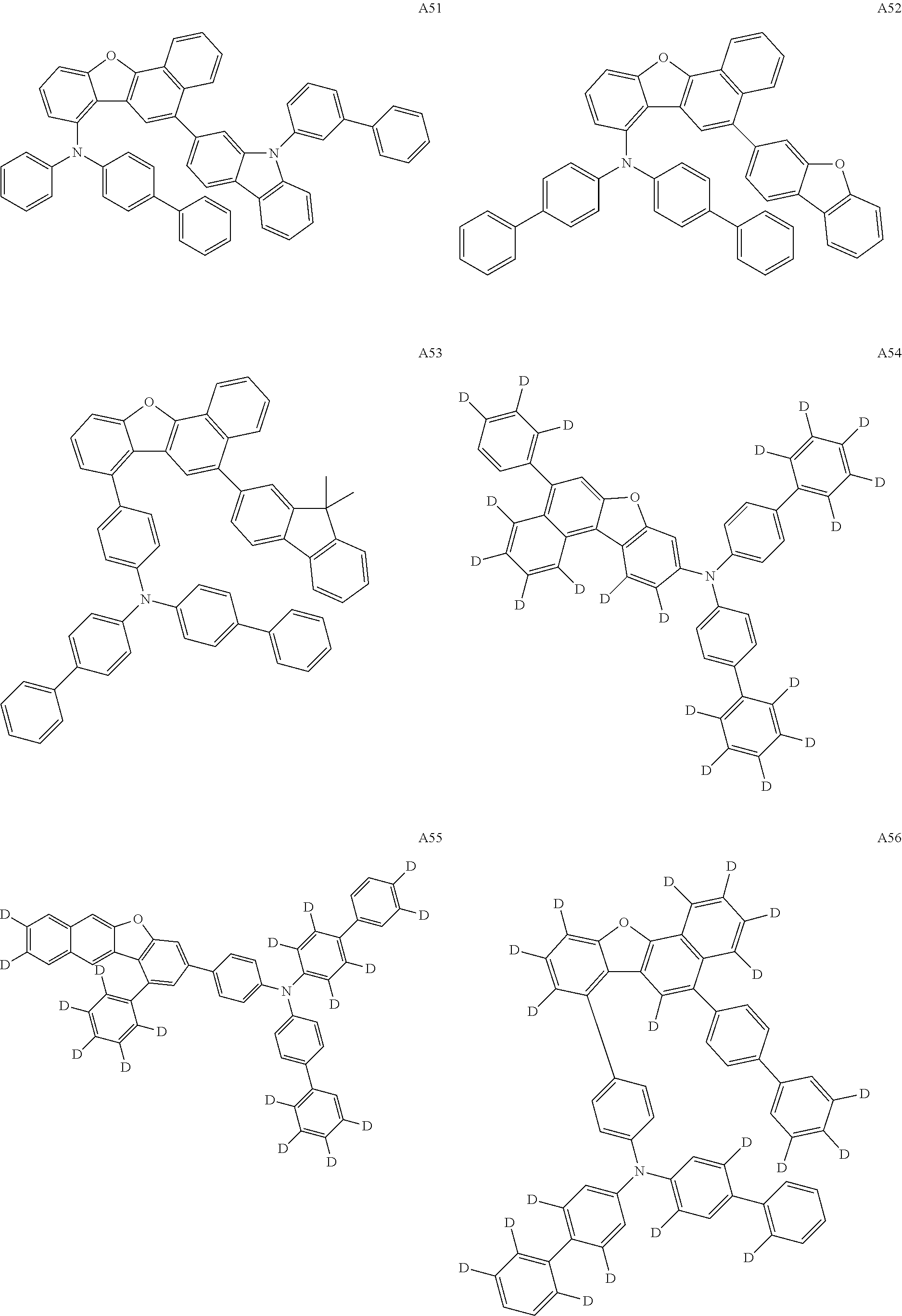

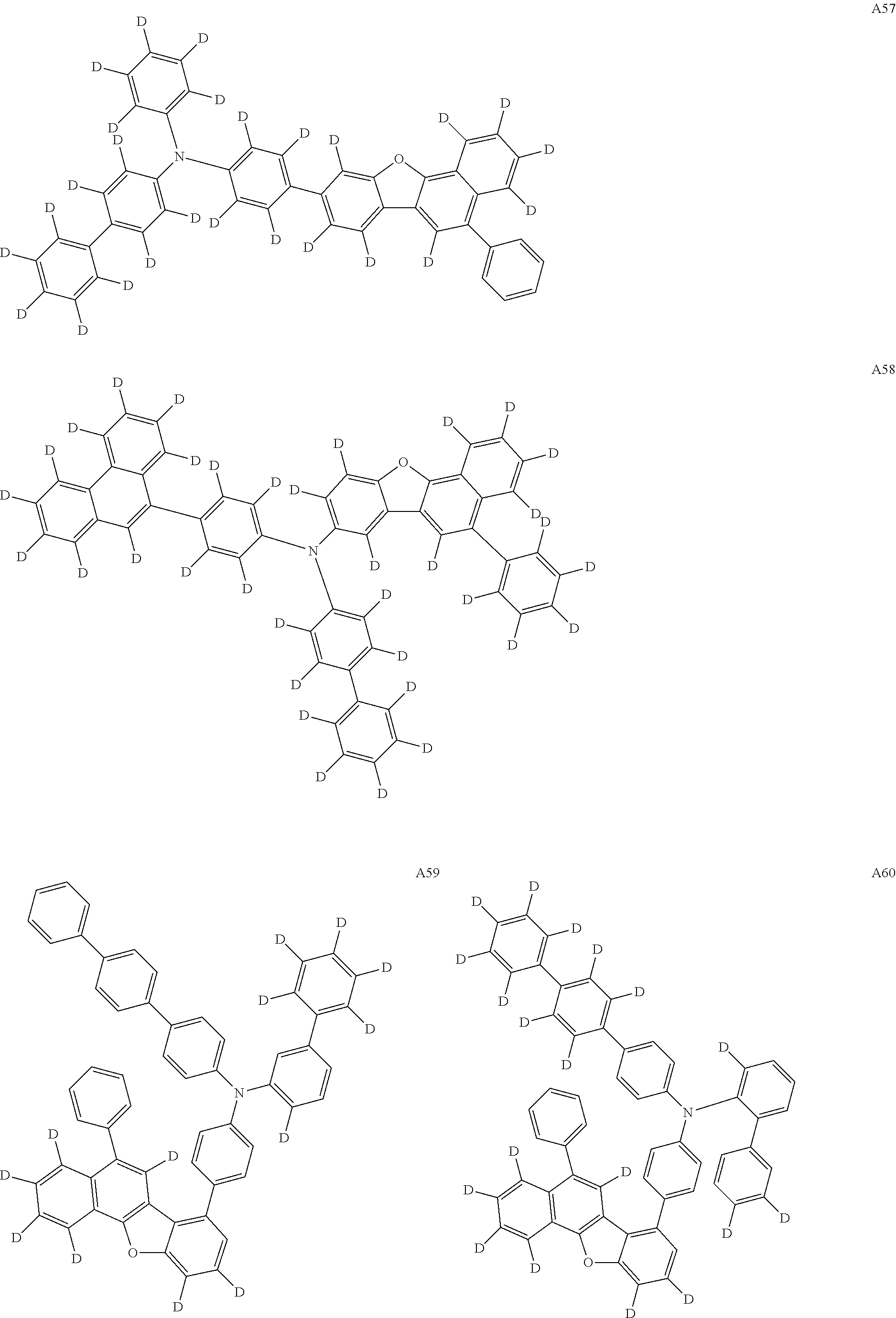

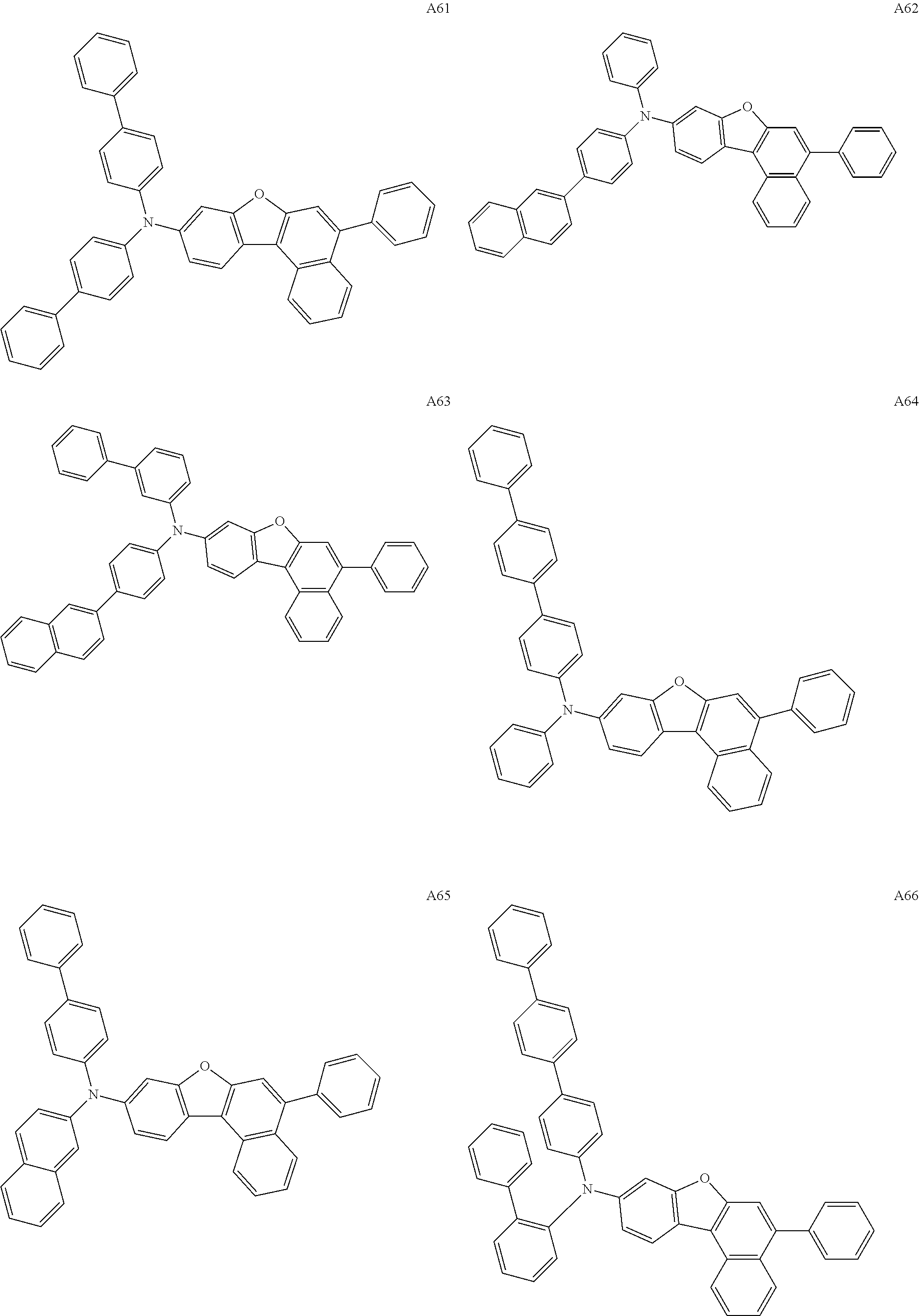

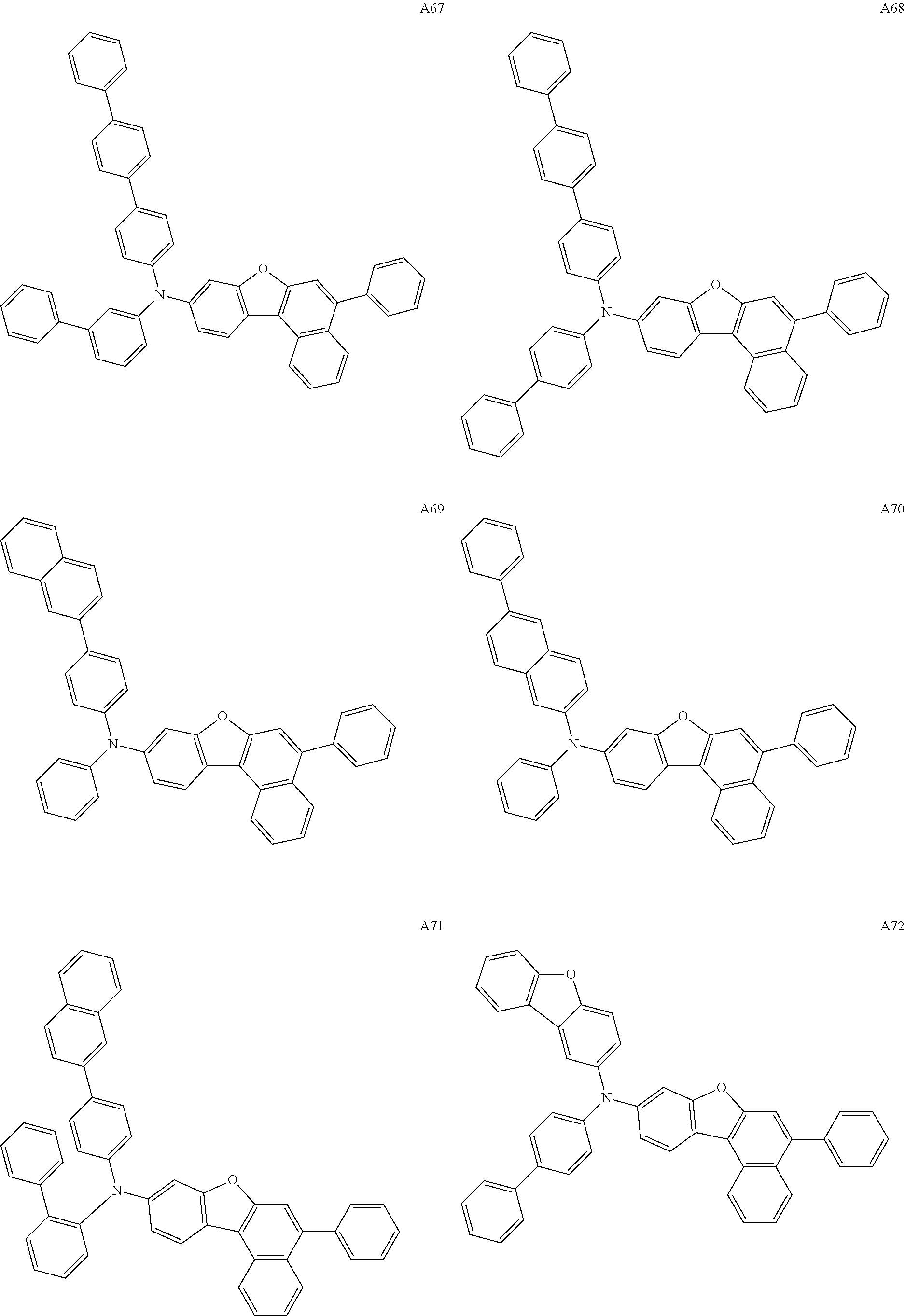

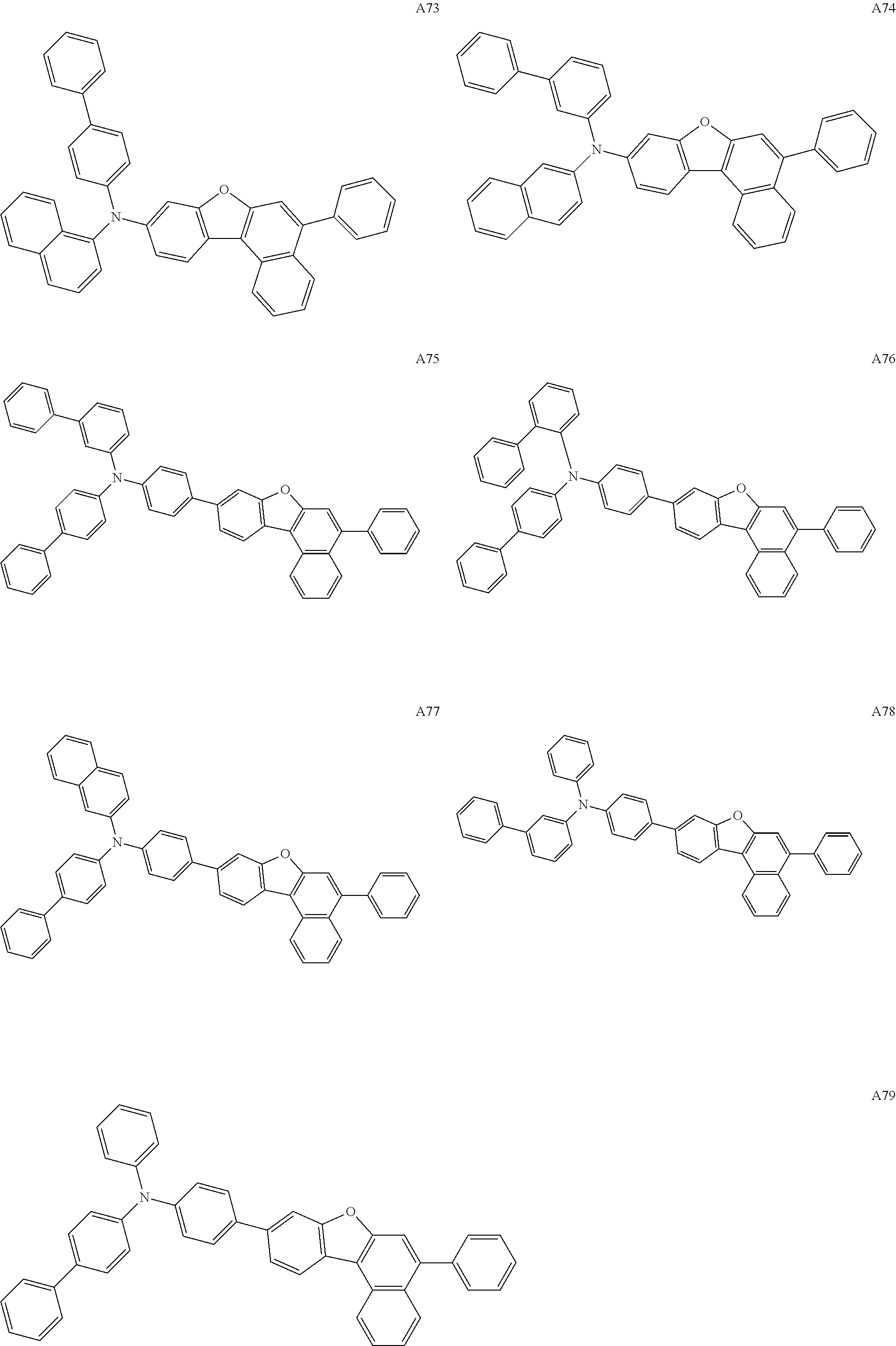

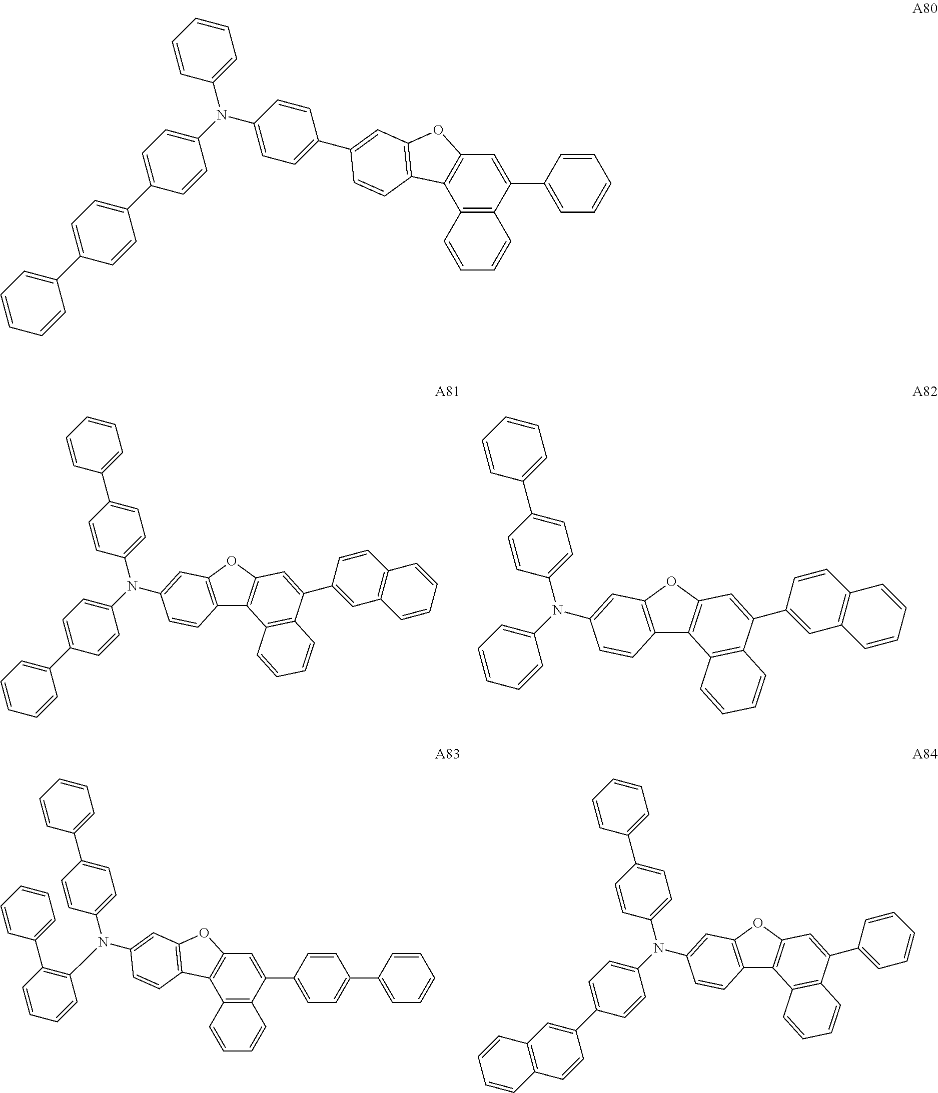 [SMILES: c1ccc(-c2ccc(-c3ccc(N(c4ccccc4)c4ccc(-c5ccc6c(c5)oc5cc(-c7ccccc7)c7ccccc7c56)cc4)cc3)cc2)cc1 ; c1ccc(-c2ccc(N(c3ccc(-c4ccccc4)cc3)c3ccc4c(c3)oc3cc(-c5ccc6ccccc6c5)c5ccccc5c34)cc2)cc1 ; c1ccc(-c2ccc(N(c3ccccc3)c3ccc4c(c3)oc3cc(-c5ccc6ccccc6c5)c5ccccc5c34)cc2)cc1 ; c1ccc(-c2ccc(-c3cc4oc5cc(N(c6ccc(-c7ccccc7)cc6)c6ccccc6-c6ccccc6)ccc5c4c4ccccc34)cc2)cc1 ; c1ccc(-c2ccc(N(c3ccc(-c4ccc5ccccc5c4)cc3)c3ccc4c(c3)oc3cc(-c5ccccc5)c5ccccc5c34)cc2)cc1]

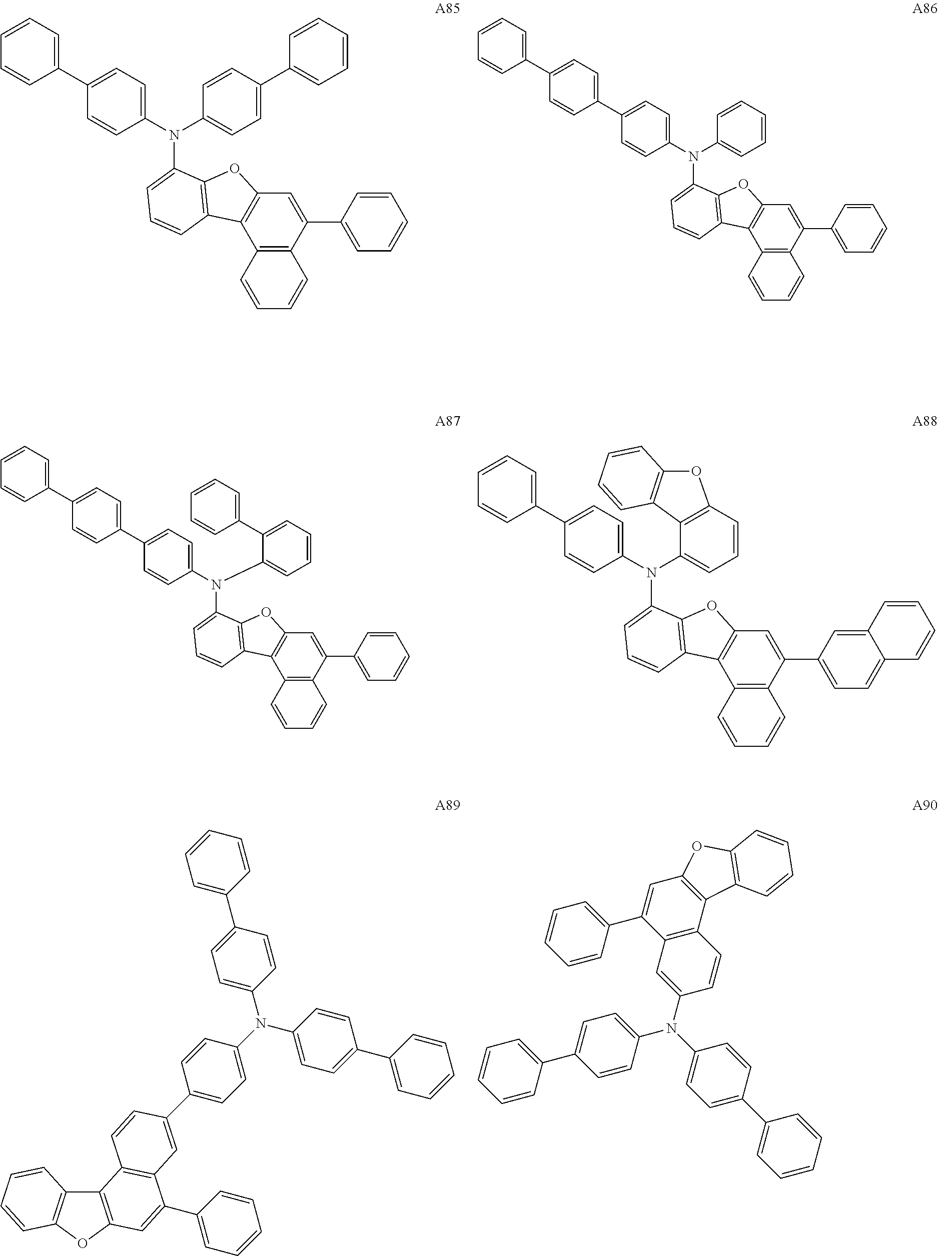

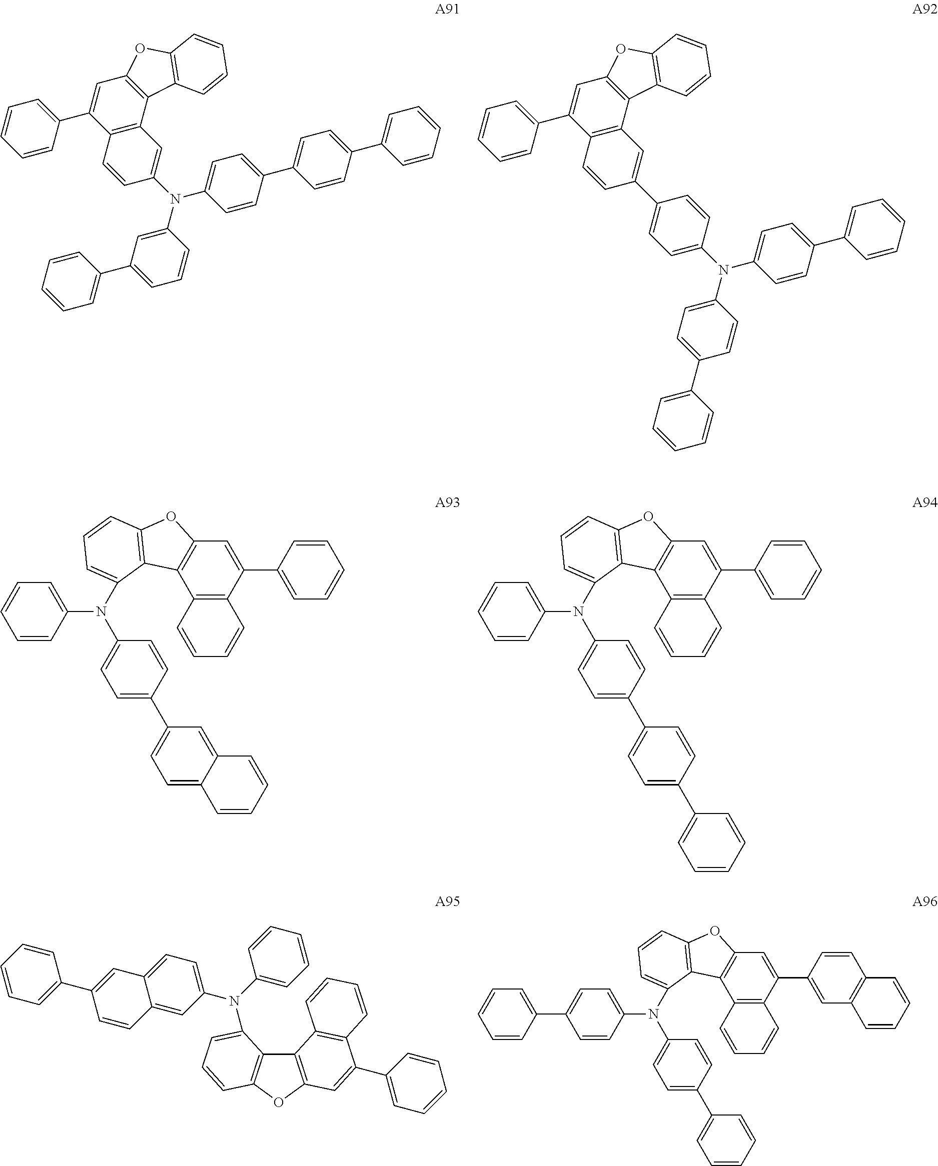

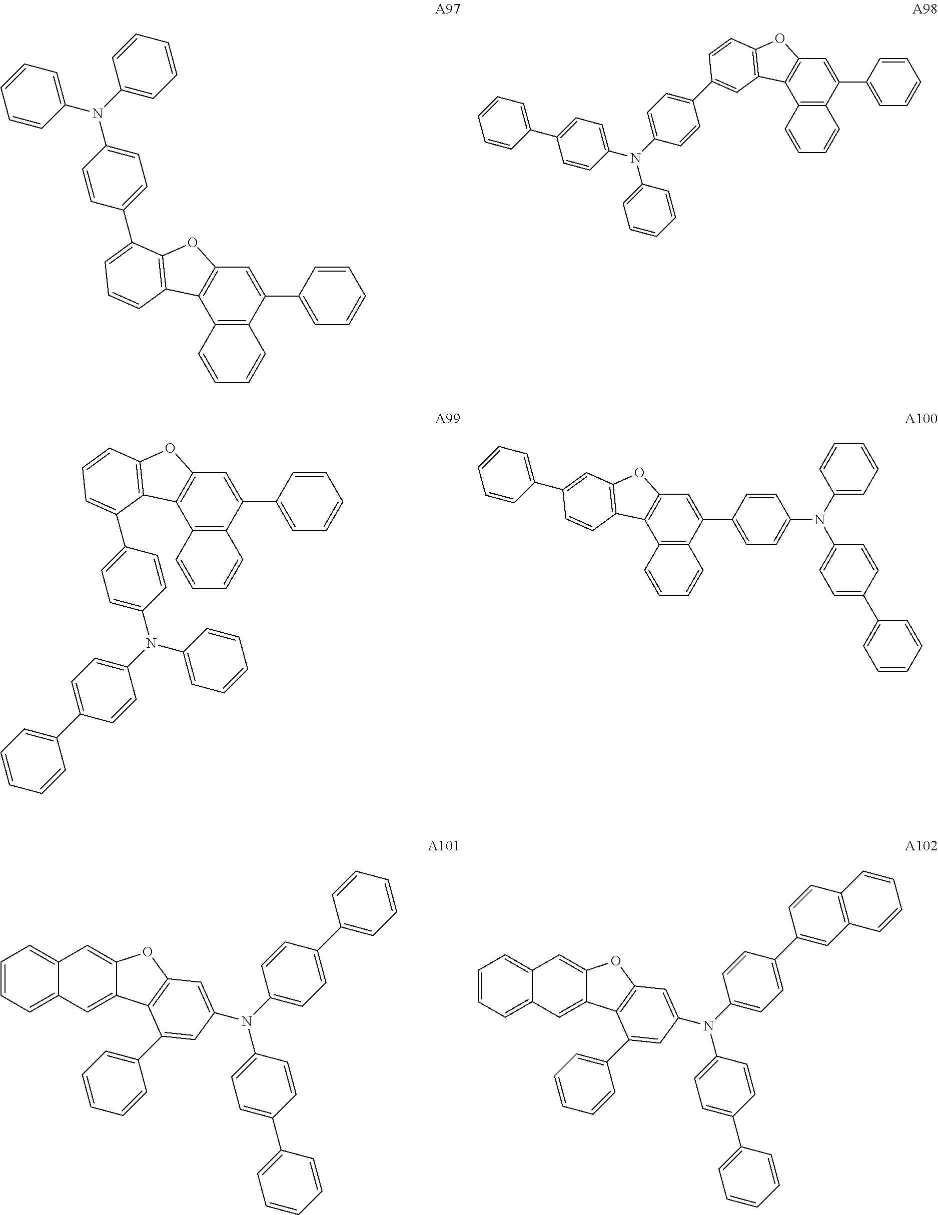 [SMILES: c1ccc(-c2cc3oc4c(-c5ccc(N(c6ccccc6)c6ccccc6)cc5)cccc4c3c3ccccc23)cc1 ; c1ccc(-c2ccc(N(c3ccccc3)c3ccc(-c4ccc5oc6cc(-c7ccccc7)c7ccccc7c6c5c4)cc3)cc2)cc1 ; c1ccc(-c2ccc(N(c3ccccc3)c3ccc(-c4cccc5oc6cc(-c7ccccc7)c7ccccc7c6c45)cc3)cc2)cc1 ; c1ccc(-c2ccc(N(c3ccccc3)c3ccc(-c4cc5oc6cc(-c7ccccc7)ccc6c5c5ccccc45)cc3)cc2)cc1 ; c1ccc(-c2ccc(N(c3ccc(-c4ccccc4)cc3)c3cc(-c4ccccc4)c4c(c3)oc3cc5ccccc5cc34)cc2)cc1 ; c1ccc(-c2ccc(N(c3ccc(-c4ccc5ccccc5c4)cc3)c3cc(-c4ccccc4)c4c(c3)oc3cc5ccccc5cc34)cc2)cc1]

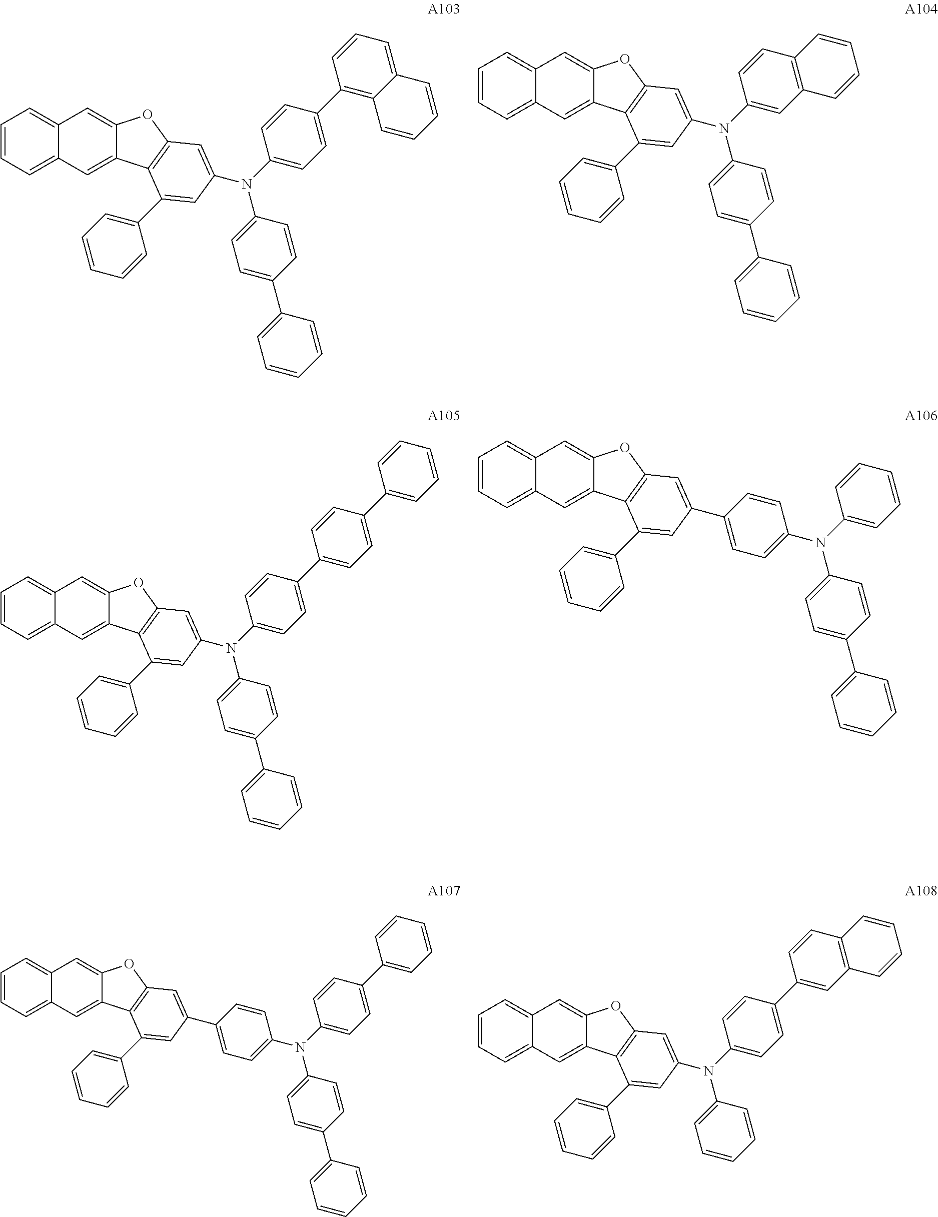

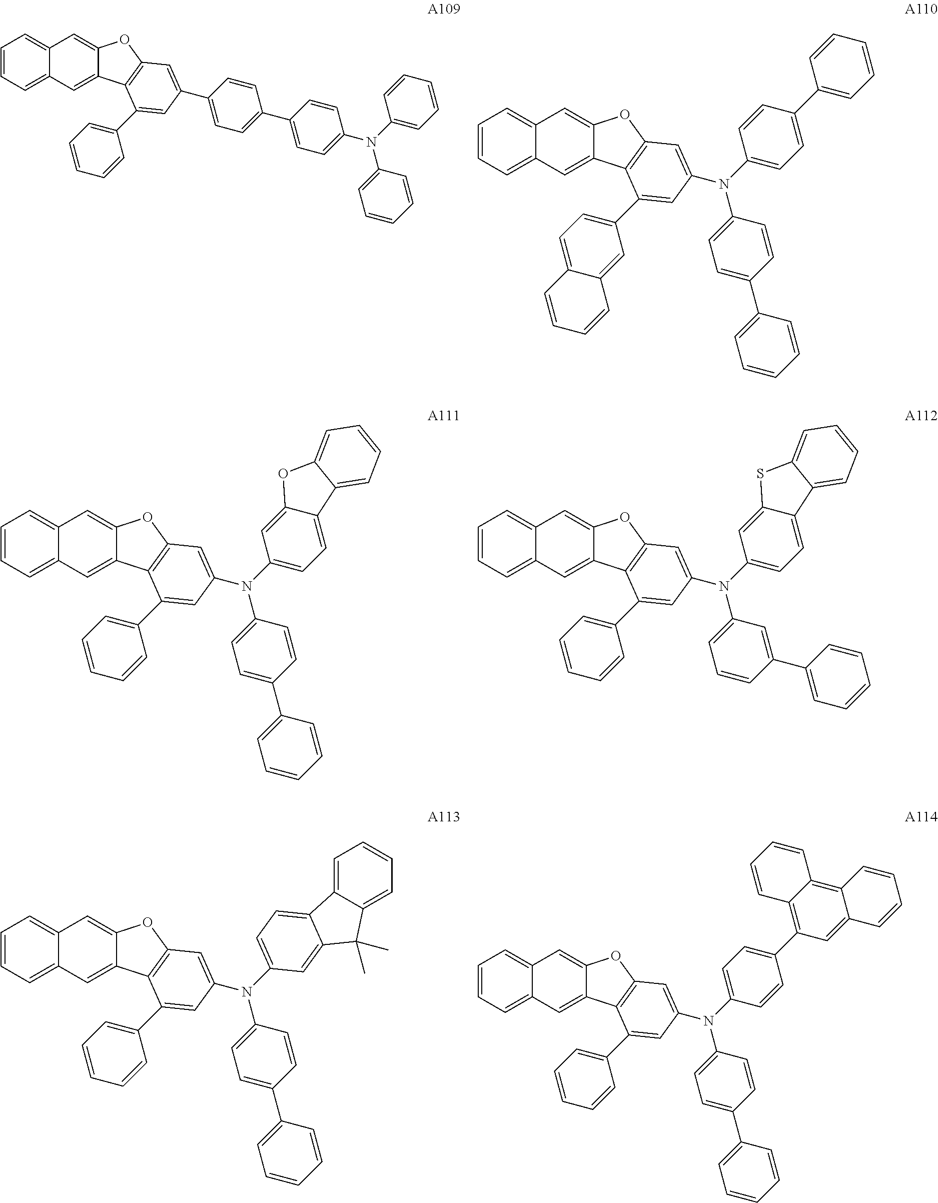

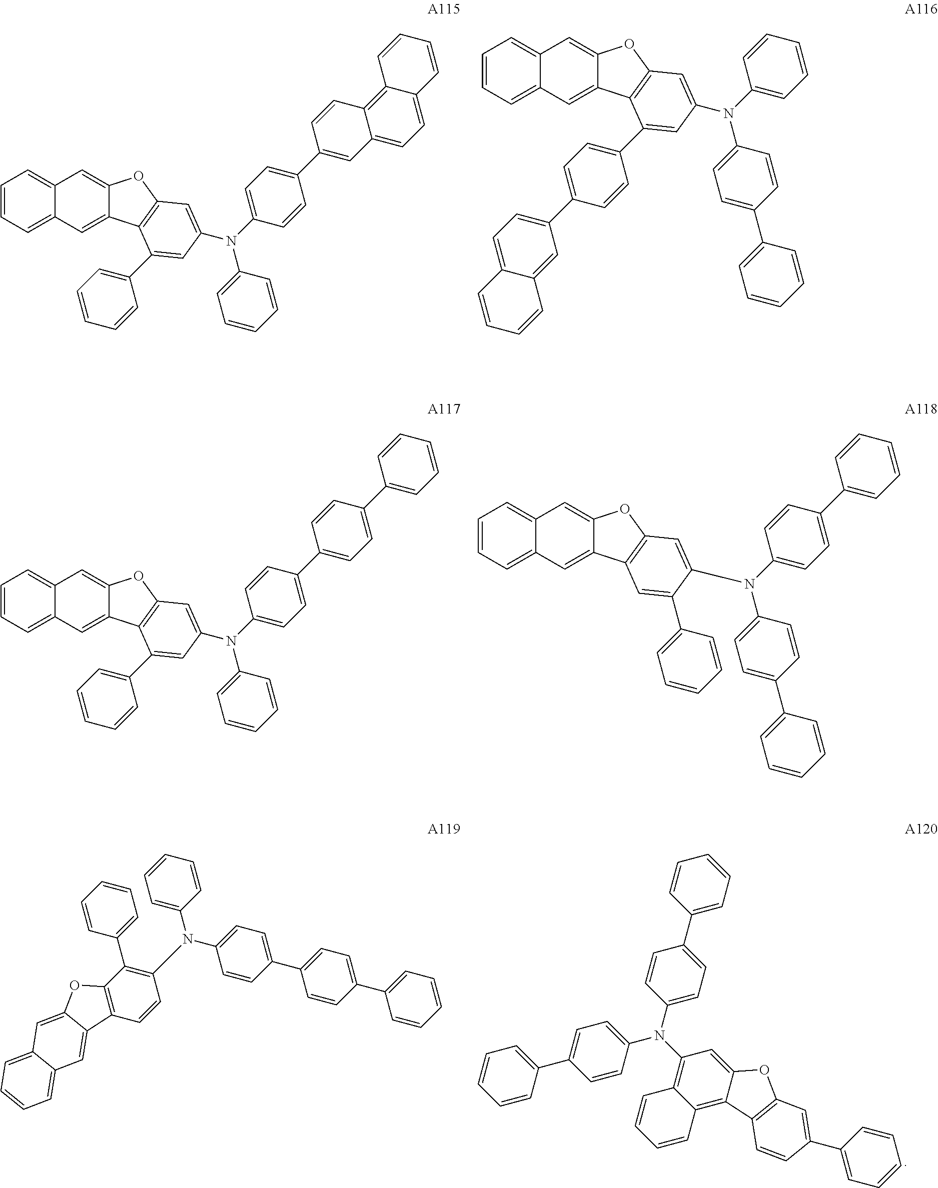

15. A composition for an organic layer of an organic light-emitting device, the composition comprising:
a heterocyclic compound represented by Formula 1 according to claim 1; and
a compound represented by Formula 3: 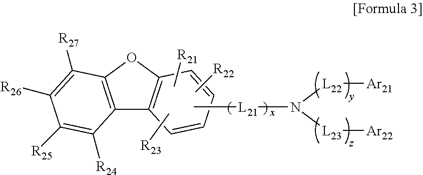
wherein, in Formula 3, two adjacent groups of R₂₁ to R₂₇ are linked to each other to form an aromatic ring represented by 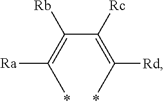
any one of the remainders of R₂₁ to R₂₇ that does not form a ring is a substituted or unsubstituted aryl group having 6 to 60 carbon atoms, or a substituted or unsubstituted heteroaryl group having 2 to 60 carbon atoms, and
Ra, Rb, Rc, Rd, and the remaining substituents are each independently selected from the group consisting of hydrogen; deuterium; halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(—O)RR′, or adjacent groups are linked to form a substituted or unsubstituted 5- to 9-membered saturated or unsaturated ring,
L₂₁ to L₂₃ are each independently a direct linkage, a substituted or unsubstituted arylene group having 6 to 60 carbon atoms, or a substituted or unsubstituted heteroarylene group having 2 to 60 carbon atoms,
x, y, and z are each independently 0 or an integer from 1 to 5, and
when x is an integer from 2 to 5, a plurality of L₂₁ groups are the same or different,
when y is an integer from 2 to 5, a plurality of L₂₂ groups are the same or different,
when z is an integer from 2 to 5, a plurality of L₂₃ groups are the same or different, and
Ar₂₁ and Ar₂₂ are each independently a substituted or unsubstituted aryl group having 6 to 60 carbon atoms, or a substituted or unsubstituted heteroaryl group having 2 to 60 carbon atoms.

16. The composition according to claim 15, wherein the ratio of the weight of the compound represented by Formula 3 to the weight of the heterocyclic compound represented by Formula 1 in the total weight of the composition is 0.1 to 10.
